(19) United States
(12) Patent Application Publication
(10) Pub. No.: US 2025/0304613 A1
(43) Pub. Date: Oct. 2, 2025

(54) AMINOOXY CLICK CHEMISTRY (AOCC): A VERSATILE CONJUGATION APPROACH

(71) Applicant: ALNYLAM PHARMACEUTICALS, INC., Cambridge, MA (US)

(72) Inventors: Muthiah MANOHARAN, Cambridge, MA (US); Shohel MORI, Cambridge, MA (US); Dhrubajyoti DATTA, Cambridge, MA (US)

(73) Assignee: ALNYLAM PHARMACEUTICALS, INC., Cambridge, MA (US)

(21) Appl. No.: 18/576,561

(22) PCT Filed: Jul. 8, 2022
(86) PCT No.: PCT/US2022/036538

§ 371 Date: Jan. 4, 2024

Related U.S. Application Data
(60) Provisional application No. 63/220,333, filed on Jul. 9, 2021.
(60) Provisional application No. 63/247,067, filed on Sep. 22, 2021.
(60) Provisional application No. 63/324,907, filed on Mar. 29, 2022.

(51) Int. Cl. C07H 21/04; A61K 31/713; C12N 15/113

(52) U.S. Cl. CPC C07H 21/04; A61K 31/713; C12N 15/113; C12N 2310/14; C12N 2310/31

(57) ABSTRACT

The present disclosure relates generally to monomers and methods for conjugating one or more ligands to oligonucleotides.

### CROSS-REFERENCE TO RELATED APPLICATIONS

This application is a 35 U.S.C. § 371 National Phase Entry Application of International Application No. PCT/US2022/036538 filed Jul. 8, 2022, which designates the U.S. and claims benefit under 35 U.S.C. § 119(e) of U.S. Provisional Application No. 63/324,907 filed on Mar. 29, 2022, U.S. Provisional Application No. 63/247,067 filed on Sep. 22, 2021 and U.S. Provisional Application No. 63/220,333 filed on Jul. 9, 2021, the contents of all of which are incorporated herein by reference in their entireties.

### TECHNICAL FIELD

The present disclosure relates generally to monomers and methods for conjugating one or more ligands to oligonucleotides.

### BACKGROUND

There is a need in the art for monomers and methods for conjugating ligands to oligonucleotides. The present disclosure addresses these needs.

### SUMMARY

In one aspect, provided herein is a compound selected from the group consisting of formulae Ia-Ig and Ii, where:

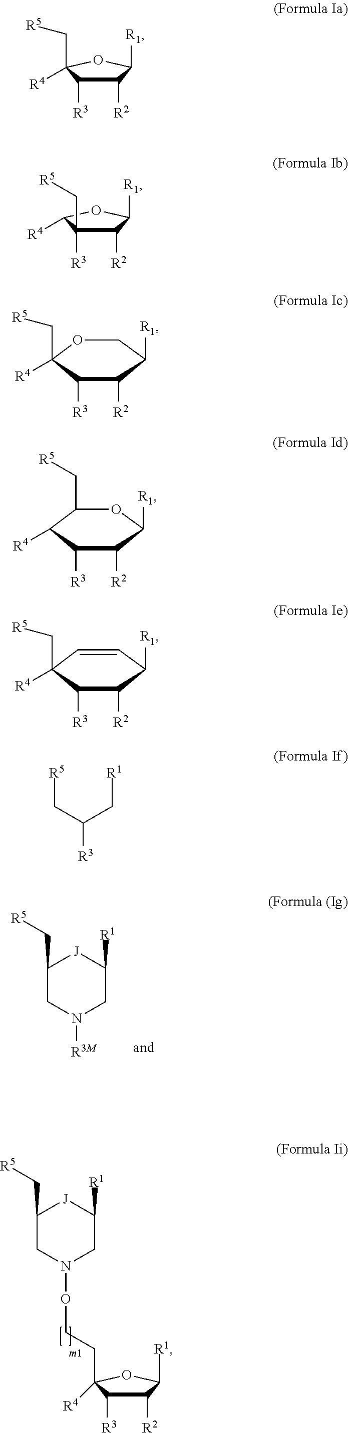

wherein:
   m1 is 0, 1, 2 or 3 (e.g., m1 is 0 or 1);
   J is O, S, CH₂ or N-alkyl (e.g., NCH₃);
   R¹ is B or —B-L¹-R⁶;
   L¹ is a linker;
   B is a nucleobase;
   R² is hydrogen, hydroxyl, halogen, protected hydroxyl, phosphate group, a reactive phosphorous group, optionally substituted C₁₋₃₀ alkyl, optionally substituted C₁₋₃₀haloalkyl, optionally substituted C₂₋₃₀alkenyl, optionally substituted C₂₋₃₀alkynyl, optionally substituted C₁₋₃₀ alkoxy (e.g., methoxy, 2-methoxyethoxy, dimethylaminoethoxyethyoxy, N-methylmethoxyamido), alkoxyalkyl (e.g., methoxyethyl), alkoxyalkylamine, alkoxyoxycarboxylate, amino, alkylamino, dialkylamino, —O—C₄₋₃₀alkyl-ON(CH₂R⁸)(CH₂R⁹), —O—C₄₋₃₀alkyl-ON(CH₂R⁸)(CH₂R⁹), a solid support, a linker, a linker covalently bonded (e.g., —OC(O)CH₂CH₂C(O)—) to a solid support, or —Z-L²-R⁶;
      each Z is independently absent, a bond, O, S, or NR^{NR6}.
         each R^{NR6} is independently H, optionally substituted C₁₋₃₀ alkyl, optionally substituted C₁₋₃₀haloalkyl, optionally substituted C₂₋₃₀alkenyl, optionally substituted C₂₋₃₀alkynyl, or a nitrogen protecting group;
      each L² is a linker;
R³ is hydrogen, hydroxyl, halogen, protected hydroxyl, phosphate group, a reactive phosphorous group, optionally substituted C₁₋₃₀ alkyl, optionally substituted C₁₋₃₀haloalkyl, optionally substituted C₂₋₃₀alkenyl, optionally substituted C₂₋₃₀alkynyl, optionally substituted C₁₋₃₀ alkoxy (e.g., methoxy, 2-methoxyethoxy, dimethylaminoethoxyethyoxy, N-methylmethoxyamido), alkoxyalkyl (e.g., methoxyethyl), alkoxyalkylamine, alkoxyoxycarboxylate, amino, alkylamino, dialkylamino, —O—C₄₋₃₀alkyl-ON(CH₂R⁸)(CH₂R⁹), —O—C₄₋₃₀alkyl-ON(CH₂R⁸)(CH₂R⁹), a solid support, a linker, a linker covalently bonded (e.g., —OC(O)CH₂CH₂C(O)—) to a solid support, or —Z-L²-R⁶, and optionally, only one of R³ and R³ is a phosphate group, a reactive phosphorous group, a solid support, a linker, or a linker covalently bonded (e.g., —OC(O)CH₂CH₂C(O)—) to a solid support;
R⁴ is hydrogen, optionally substituted C₁₋₆alkyl, optionally substituted C₁₋₃₀haloalkyl, optionally substituted C₂₋₆alkenyl, optionally substituted C₂₋₆alkynyl, optionally substituted C₁₋₆alkoxy, or —Z-L²-R⁶;
or R⁴ and R² taken together are 4′-C(R¹⁰R¹¹)ᵥ—Y-2′ or 4′-Y—C(R¹⁰R¹¹)ᵥ-2′;
   Y is —O—, —CH₂—, —CH(Me)-, —C(CH₃)₂—, —S—, —N(R¹²)—, —C(O)—, —C(S)—, —S(O)—, —S(O)₂—, —OC(O)—, —C(O)O—, —N(R¹²)C(O)—, or —C(O)N(R¹²)—;
   R¹⁰ and R¹¹ independently are H, optionally substituted C₁-C₆alkyl, optionally substituted C₁₋₃₀haloalkyl, optionally substituted C₂-C₆alkenyl or optionally substituted C₂-C₆alkynyl;
   R¹² is hydrogen, optionally substituted C₁₋₃₀alkyl, optionally substituted C₁-C₃₀alkoxy, C₁₋₄haloalkyl, optionally substituted C₂₋₄alkenyl, optionally substituted C₂₋₄alkynyl, optionally substituted C₁₋₃₀alky-CO₂H, or a nitrogen-protecting group;
   v is 1, 2 or 3;
or R⁴ and R³ taken together with the atoms to which they are attached form an optionally substituted C₃₋₈cycloalkyl, optionally substituted C₃₋₈cycloalkenyl, or optionally substituted 3-8 membered heterocyclyl;
R⁵ is R⁶, —Z-L²-R⁶, hydrogen, hydroxyl, protected hydroxyl, phosphate group, optionally substituted C₁₋₃₀ alkyl, optionally substituted C₁₋₃₀haloalkyl, optionally substituted C₂₋₃₀alkenyl, optionally substituted C₂₋₃₀alkynyl, optionally substituted C₁₋₃₀ alkoxy, halogen, alkoxyalkyl (e.g., methoxyethyl), alkoxyalkylamine, alkoxyoxycarboxylate, amino, alkylamino, dialkylamino, —O—C₄₋₃₀alkyl-ON(CH₂R⁸)(CH₂R⁹), —O—C₄₋₃₀alkyl-ON(CH₂R⁸)(CH₂R⁹), vinylphosphonate (VP) group, C₃₋₆cycloalkylphosphonate (e.g., cyclopropylphosphonate), monophosphate ((HO)₂(O)P—O-5′), diphosphate ((HO)₂(O)P—O—P(HO)(O)—O-5′), triphosphate ((HO)₂(O)P—O—(HO)(O)P—O—P(HO)(O)—O-5′); monothiophosphate (phosphorothioate, (HO)2(S)P—O-5′), monodithiophosphate (phosphorodithioate; (HO)(HS)(S)P—O-5′), phosphorothiolate ((HO)2(O)P—S-5′); alpha-thiotriphosphate; beta-thiotriphosphate; gamma-thiotriphosphate;
phosphoramidates ((HO)₂(O)P—NH-5′, (HO)(NH₂)(O)P—O-5′), alkylphosphonates (R(OH)(O)P—O-5′, R=alkyl, e.g., methyl, ethyl, isopropyl, propyl, etc. . . . ), alkyletherphosphonates (R(OH)(O)P—O-5′, R=alkylether, e.g., methoxymethyl (CH₂OMe), ethoxymethyl, etc. . . . ), (HO)₂(X)P—O[—(CH₂)ₐ—O—P(X)(OH)—O]_{b}-5′ or (HO)2(X)P—O[—(CH₂)ₐ—P(X)(OH)—O]_{b}-5′ or (HO)₂(X)P—[—(CH₂)ₐ—O—P(X)(OH)—O]_{b}-5′, where X is O, S or optionally substituted alkyl, and dialkyl terminal phosphates and phosphate mimics (e.g., HO[—(CH₂)ₐ—O—P(X)(OH)—O]_{b}-5′, H₂N[—(CH₂)ₐ—O—P(X)(OH)—O]_{b}-5′, H[—(CH₂)ₐ—O—P(X)(OH)—O]_{b}-5′, Me₂N[—(CH₂)ₐ—O—P(X)(OH)—O]_{b}-5′, HO[—(CH₂)ₐ—P(X)(OH)—O]_{b}-5′, H₂N[—(CH₂)ₐ—P(X)(OH)—O]_{b}-5′, H[—(CH₂)ₐ—P(X)(OH)—O]_{b}-5′, Me₂N[—(CH₂)ₐ—P(X)(OH)—O]_{b}-5′, wherein X is O or S; and a and b are each independently 1-10);
each R⁶ is independently —O—N(R⁷)R^{7′}, —O—N═C(R⁷)R^{7′}, ═N—OR⁷, —N(R⁷)—OR^{7′} or —N(R^{7′})—OR⁷;
each R⁷ and R^{7′} is independently H or a ligand, (e.g., a ligand selected independently from the group consisting of carbohydrates, lipids, vitamins, peptides, proteins, lipoproteins, peptidomimetics, polyamines, nucleosides and nucleotides, oligonucleotides, therapeutic agents, diagnostic agents, detectable labels, antibodies or fragments thereof, optionally substituted C₁₋₃₀ alkyl, optionally substituted C₁₋₃₀ alkenyl, optionally substituted C₁₋₃₀alkynyl, optionally substituted cycloalkyl, optionally substituted heterocyclyl, optionally substituted aryl, optionally substituted heteroaryl, polyethylene glycols (PEGs), nitrogen protecting group, optionally provided that at least one of R⁷ and R^{7′} is not H;
or R⁷ and R⁷ together with the N they are attached to form an optionally substituted heterocyclyl (e.g., phthalimide or morpholine);
each R⁸ and R⁹ is independently H, a targeting ligand (e.g., GalNac), a pharmacokinetics modifier, optionally substituted C₁₋₃₀ alkyl, optionally substituted C₁₋₃₀ alkenyl, or optionally substituted C₁₋₃₀alkynyl; and
R^{3M} is hydrogen, hydroxyl, protected hydroxyl, phosphate group, a reactive phosphorous group, optionally substituted C₁₋₃₀ alkyl, optionally substituted C₂₋₃₀alkenyl, optionally substituted C₂₋₃₀alkynyl, optionally substituted C₁₋₃₀ alkoxy (e.g., methoxy), alkoxyalkyl (e.g., methoxyethyl), alkoxyalkylamine, alkoxyoxycarboxylate, —O—C₄₋₃₀alkyl-ON(CH₂R⁸)(CH₂R⁹), —O—C₄₋₃₀alkyl-ON(CH₂R⁸)(CH₂R⁹), a solid support, a linker, or a linker covalently bonded (e.g., —OC(O)CH₂CH₂C(O)—) to a solid support, and provided that when the compound is of formulae Ia-Ig, the compound comprises at least one R⁶ group.

In another aspect, provided herein is a compound selected from the group consisting of formulae IIa-IIg, where:

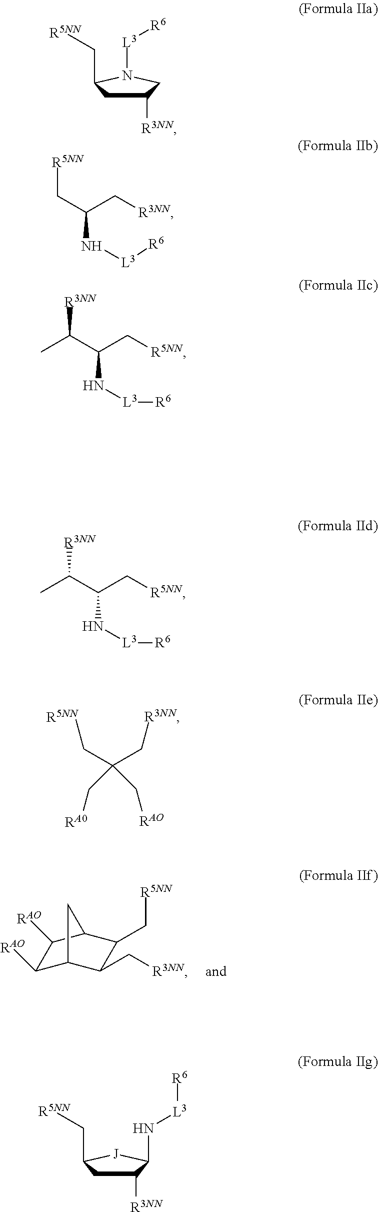

wherein:
   J is O, S, CH₂ or N-alkyl (e.g., NCH₃);
   L³ is a linker;
   R^{3NN} is independently hydrogen, hydroxyl, halogen, protected hydroxyl, phosphate group, a reactive phosphorous group, optionally substituted C₁₋₃₀ alkyl, optionally substituted C₁₋₃₀haloalkyl, optionally substituted C₂₋₃₀alkenyl, optionally substituted C₂₋₃₀alkynyl, optionally substituted C₁₋₃₀ alkoxy (e.g., methoxy, 2-methoxyethoxy, dimethylaminoethoxyethyoxy, N-methylmethoxyamido), alkoxyalkyl (e.g., methoxyethyl), alkoxyalkylamine, alkoxyoxycarboxylate, amino, alkylamino, dialkylamino, —O—C₄₋₃₀alkyl-ON(CH₂R⁸)(CH₂R⁹), —O—C₄-30alkyl-ON(CH₂R⁸)(CH₂R⁹), a solid support, a linker, or a linker covalently bonded (e.g., —OC(O)CH₂CH₂C(O)—) to a solid support;
each Z is independently absent, a bond, O, S, or NR^{NR6};
each R^{NR6} is independently H, optionally substituted C₁₋₃₀ alkyl, optionally substituted C₁₋₃₀haloalkyl, optionally substituted C₂₋₃₀alkenyl, optionally substituted C₂₋₃₀alkynyl, or a nitrogen protecting group;
each L² is a linker;
R^{5NN} is hydrogen, hydroxyl, protected hydroxyl, phosphate group, optionally substituted C₁₋₃₀ alkyl, optionally substituted C₁₋₃₀haloalkyl, optionally substituted C₂₋₃₀alkenyl, optionally substituted C₂₋₃₀alkynyl, optionally substituted C₁₋₃₀ alkoxy, halogen, alkoxyalkyl (e.g., methoxyethyl), alkoxyalkylamine, alkoxyoxycarboxylate, amino, alkylamino, dialkylamino, —O—C₄₋₃₀alkyl-ON(CH₂R⁸)(CH₂R⁹), —O—C₄₋₃₀alkyl-ON(CH₂R⁸)(CH₂R⁹), vinylphosphonate (VP) group, C₃₋₆cycloalkylphosphonate (e.g., cyclopropylphosphonate), monophosphate ((HO)₂(O)P—O-5′), diphosphate ((HO)₂(O)P—O—P(HO)(O)—O-5′), triphosphate ((HO)₂(O)P—O—(HO)(O)P—O—P(HO)(O)—O-5′); monothiophosphate (phosphorothioate, (HO)₂(S)P—O-5′), monodithiophosphate (phosphorodithioate; (HO)(HS)(S)P—O-5′), phosphorothiolate ((HO)2(O)P—S-5′); alpha-thiotriphosphate; beta-thiotriphosphate; gamma-thiotriphosphate; phosphoramidates ((HO)₂(O)P—NH-5′, (HO)(NH₂)(O)P—O-5′), alkylphosphonates (R(OH)(O)P—O-5′, R=alkyl, e.g., methyl, ethyl, isopropyl, propyl, etc. . . . ), alkyletherphosphonates (R(OH)(O)P—O-5′, R=alkylether, e.g., methoxymethyl (CH₂OMe), ethoxymethyl, etc. . . . ), (HO)₂(X)P—O[—(CH₂)ₐ—O—P(X)(OH)—O]_{b}-5′ or (HO)₂(X)P—O[—(CH₂)ₐ—P(X)(OH)—O]_{b}-5′ or (HO)₂(X)P—[—(CH₂)ₐ—O—P(X)(OH)—O]_{b}-5′, where X is O, S or optionally substituted alkyl, and dialkyl terminal phosphates and phosphate mimics (e.g., HO[—(CH₂)ₐ—O—P(X)(OH)—O]_{b}-5′, H₂N[—(CH₂)ₐ—O—P(X)(OH)—O]_{b}-5′, H[—(CH₂)ₐ—O—P(X)(OH)—O]_{b}-5′, Me₂N[—(CH₂)ₐ—O—P(X)(OH)—O]_{b}-5′, HO[—(CH₂)ₐ—P(X)(OH)—O]_{b}-5′, H₂N[—(CH₂)ₐ—P(X)(OH)—O]_{b}-5′, H[—(CH₂)ₐ—P(X)(OH)—O]_{b}-5′, Me₂N[—(CH₂)ₐ—P(X)(OH)—O]_{b}-5′, wherein X is O or S; and a and b are each independently 1-10);
each R^{AO} is independently R⁶ or —Z-L²-R⁶;
each R⁶ is independently —O—N(R⁷)R^{7′}, —O—N═C(R⁷)R^{7′}, =N—OR⁷, —N(R⁷)—OR^{7′} or —N(R^{7′})—OR⁷; and
each R⁷ and R^{7′} is independently H or a ligand, (e.g., a ligand selected independently from the group consisting of carbohydrates, lipids, vitamins, peptides, proteins, lipoproteins, peptidomimetics, polyamines, nucleosides and nucleotides, oligonucleotides, therapeutic agents, diagnostic agents, detectable labels, antibodies or fragments thereof, optionally substituted C₁₋₃₀ alkyl, optionally substituted C₁₋₃₀ alkenyl, optionally substituted C₁₋₃₀alkynyl, optionally substituted cycloalkyl, optionally substituted heterocyclyl, optionally substituted aryl, optionally substituted heteroaryl, polyethylene glycols (PEGs), nitrogen protecting group, optionally provided that at least one of R⁷ and R^{7′} is not H.

In some embodiments of any one of the aspects described herein, the compound is of Formula Ia.

The compounds of Formulae Ia-Ig, Ii and IIa-IIg are useful in the synthesis oligonucleotides. Accordingly, in another aspect, provided herein is an oligonucleotide prepared using a compound selected from compounds of formulae Ia-Ig, Ii and IIa-IIf. For example, an oligonucleotide comprising at least one nucleoside selected from the group consisting of nucleotides of formulae IIIa-IIIh and IVa-IVg, where:

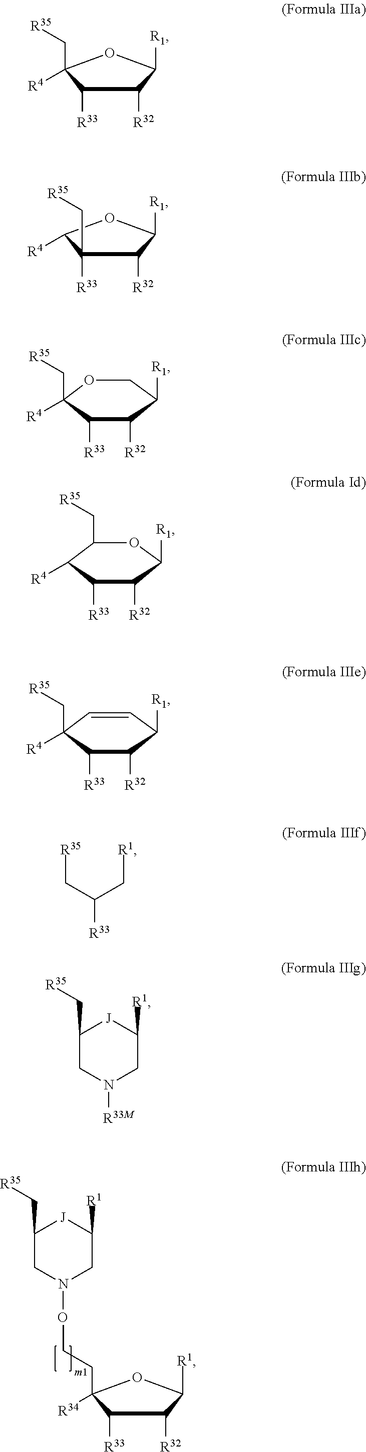

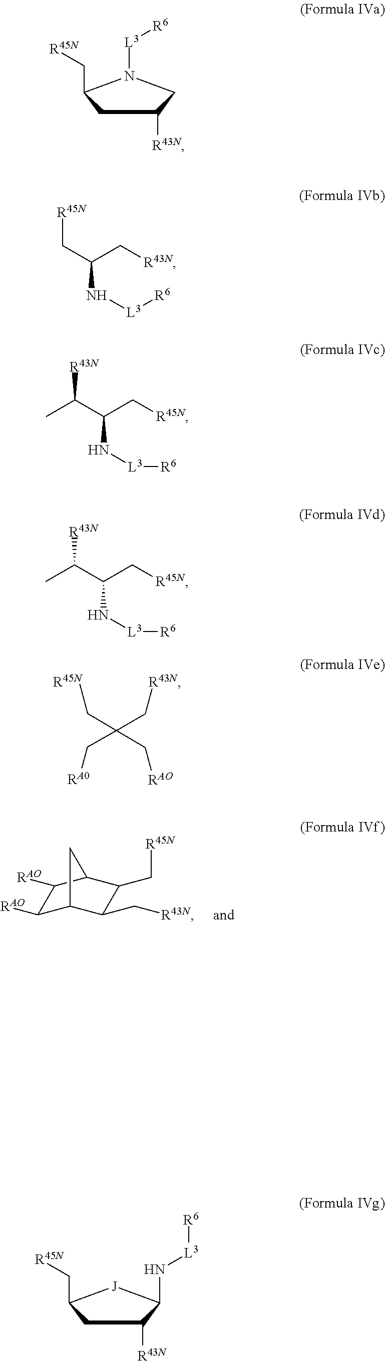

In nucleoside of formulae IIIa-IIIh and IVa-IVg,
m1 is 0, 1, 2 or 3 (e.g., m1 is 0 or 1);
J is O, S, CH₂ or N-alkyl (e.g., NCH₃);
R¹ is B or —B-L¹-R⁶;
L¹ is a linker;
L³ is a linker;
B is a nucleobase;
R³² is hydrogen, hydroxyl, halogen, protected hydroxyl, phosphate group, a reactive phosphorous group, optionally substituted C₁₋₃₀ alkyl, optionally substituted C₁₋₃₀haloalkyl, optionally substituted C₂₋₃₀alkenyl, optionally substituted C₂₋₃₀alkynyl, optionally substituted C₁₋₃₀ alkoxy (e.g., methoxy, 2-methoxyethoxy, dimethylaminoethoxyethyoxy, N-methylmethoxyamido), alkoxyalkyl (e.g., methoxyethyl), alkoxyalkylamine, alkoxyoxycarboxylate, amino, alkylamino, dialkylamino, —O—C₄₋₃₀alkyl-ON(CH₂R⁸)(CH₂R⁹), —O—C₄₋₃₀alkyl-ON(CH₂R⁸)(CH₂R⁹), a bond to an internucleotide linkage to a subsequent nucleotide, a 3′-oligonuclotide capping group, a ligand, a solid support, a linker, a linker covalently bonded (e.g., —OC(O)CH₂CH₂C(O)—) to a solid support, or —Z-L²-R⁶;
each Z is independently absent, a bond, O, S, or NR^{NR6}.
each R^{NR6} is independently H, optionally substituted C₁₋₃₀ alkyl, optionally substituted C₁₋₃₀haloalkyl, optionally substituted C₂₋₃₀alkenyl, optionally substituted C₂₋₃₀alkynyl, or a nitrogen protecting group;
each L² is a linker;
R³³ is hydrogen, hydroxyl, halogen, protected hydroxyl, phosphate group, a reactive phosphorous group, optionally substituted C₁₋₃₀ alkyl, optionally substituted C₁₋₃₀haloalkyl, optionally substituted C₂₋₃₀alkenyl, optionally substituted C₂₋₃₀alkynyl, optionally substituted C₁₋₃₀ alkoxy (e.g., methoxy, 2-methoxyethoxy, dimethylaminoethoxyethyoxy, N-methylmethoxyamido), alkoxyalkyl (e.g., methoxyethyl), alkoxyalkylamine, alkoxyoxycarboxylate, amino, alkylamino, dialkylamino, —O—C₄₋₃₀alkyl-ON(CH₂R⁸)(CH₂R⁹), —O—C₄₋₃₀alkyl-ON(CH₂R⁸)(CH₂R⁹), a bond to an internucleotide linkage to a subsequent nucleotide, a 3′-oligonuclotide capping group, a ligand, a solid support, a linker, a linker covalently bonded (e.g., —OC(O)CH₂CH₂C(O)—) to a solid support, or —Z-L²-R⁶, and optionally, only one of R³ and R³ is a phosphate group, a reactive phosphorous group, a solid support, a linker, a linker covalently bonded (e.g., —OC(O)CH₂CH₂C(O)—) to a solid support, or a bond to an internucleotide linkage to a subsequent nucleotide;
R⁴ is hydrogen, optionally substituted C₁₋₆alkyl, optionally substituted C₁₋₃₀haloalkyl, optionally substituted C₂₋₆alkenyl, optionally substituted C₂₋₆alkynyl, optionally substituted C₁₋₆alkoxy, or —Z-L²-R⁶;
or R⁴ and R³² taken together are 4′-C(R¹⁰R¹¹)ᵥ—Y-2′ or 4′-Y—C(R¹⁰R¹¹)ᵥ-2′;
   Y is —O—, —CH₂—, —CH(Me)-, —C(CH₃)₂—, —S—, —N(R¹²)—, —C(O)—, —C(S)—, —S(O)—, —S(O)₂—, —OC(O)—, —C(O)O—, —N(R¹²)C(O)—, or —C(O)N(R¹²)—;
   R¹⁰ and R¹¹ independently are H, optionally substituted C₁-C₆alkyl, optionally substituted C₂-C₆alkenyl or optionally substituted C₂-C₆alkynyl;
   R¹² is hydrogen, optionally substituted C₁₋₃₀alkyl, optionally substituted C₁₋₃₀haloalkyl, optionally substituted C₁-C₃₀alkoxy, C₁₋₄haloalkyl, optionally substituted C₂₋₄alkenyl, optionally substituted C₂₋₄alkynyl, optionally substituted C₁₋₃₀alky-CO₂H, or a nitrogen-protecting group;
   v is 1, 2 or 3;
or R⁴ and R³³ taken together with the atoms to which they are attached form an optionally substituted C₃₋₈cycloalkyl, optionally substituted C₃₋₈cycloalkenyl, or optionally substituted 3-8 membered heterocyclyl;
R³⁵ is R⁶, —Z-L²-R⁶, a bond to an internucleotide linkage to a preceding nucleotide, hydrogen, hydroxyl, protected hydroxyl, phosphate group, optionally substituted C₁₋₃₀ alkyl, optionally substituted C₁₋₃₀haloalkyl, optionally substituted C₂₋₃₀alkenyl, optionally substituted C₂₋₃₀alkynyl, optionally substituted C₁₋₃₀ alkoxy, halogen, alkoxyalkyl (e.g., methoxyethyl), alkoxyalkylamine, alkoxyoxycarboxylate, amino, alkylamino, dialkylamino, —O—C₄₋₃₀alkyl-ON(CH₂R⁸)(CH₂R⁹), —O—C₄₋₃₀alkyl-ON(CH₂R⁸)(CH₂R⁹), vinylphosphonate (VP) group, C₃₋₆cycloalkylphosphonate (e.g., cyclopropylphosphonate), monophosphate ((HO)₂(O)P—O-5′), diphosphate ((HO)₂(O)P—O—P(HO)(O)—O-5′), triphosphate ((HO)₂(O)P—O—(HO)(O)P—O—P(HO)(O)—O-5′); monothiophosphate (phosphorothioate, (HO)₂(S)P—O-5′), monodithiophosphate (phosphorodithioate; (HO)(HS)(S)P—O-5′), phosphorothiolate ((HO)2(O)P—S-5′); alpha-thiotriphosphate; beta-thiotriphosphate; gamma-thiotriphosphate;
phosphoramidates ((HO)₂(O)P—NH-5′, (HO)(NH₂)(O)P—O-5′), alkylphosphonates (R(OH)(O)P—O-5′, R=alkyl, e.g., methyl, ethyl, isopropyl, propyl, etc. . . . ), alkyletherphosphonates (R(OH)(O)P—O-5′, R=alkylether, e.g., methoxymethyl (CH₂OMe), ethoxymethyl, etc. . . . ), (HO)₂(X)P—O[—(CH₂)ₐ—O—P(X)(OH)—O]_{b}-5′ or (HO)2(X)P—O[—(CH₂)ₐ—P(X)(OH)—O]_{b}-5′ or (HO)2(X)P—[—(CH₂)ₐ—O—P(X)(OH)—O]_{b}-5′, where X is O, S or optionally substituted alkyl, and dialkyl terminal phosphates and phosphate mimics (e.g., HO[—(CH₂)ₐ—O—P(X)(OH)—O]_{b}-5′, H₂N[—(CH₂)ₐ—O—P(X)(OH)—O]_{b}-5′, H[—(CH₂)ₐ—O—P(X)(OH)—O]_{b}-5′, Me₂N[—(CH₂)ₐ—O—P(X)(OH)—O]_{b}-5′, HO[—(CH₂)ₐ—P(X)(OH)—O]_{b}-5′, H₂N[—(CH₂)ₐ—P(X)(OH)—O]_{b}-5′, H[—(CH₂)ₐ—P(X)(OH)—O]_{b}-5′, Me₂N[—(CH₂)ₐ—P(X)(OH)—O]_{b}-5′, wherein X is O or S; and a and b are each independently 1-10);
each R⁶ is independently —O—N(R⁷)R^{7′}, —O—N═C(R⁷)R^{7′}, =N—OR⁷, —N(R⁷)—OR^{7′} or —N(R^{7′})—OR⁷;
each R⁷ and R^{7′} is independently H or a ligand, (e.g., a ligand selected independently from the group consisting of carbohydrates, lipids, vitamins, peptides, proteins, lipoproteins, peptidomimetics, polyamines, nucleosides and nucleotides, oligonucleotides, therapeutic agents, diagnostic agents, detectable labels, antibodies or fragments thereof, optionally substituted C₁₋₃₀ alkyl, optionally substituted C₁₋₃₀ alkenyl, optionally substituted C₁₋₃₀alkynyl, optionally substituted cycloalkyl, optionally substituted heterocyclyl, optionally substituted aryl, optionally substituted heteroaryl, polyethylene glycols (PEGs), nitrogen protecting group, optionally provided that at least one of R⁷ and R^{7′} is not H;
or R⁷ and R^{7′} together with the N they are attached to form an optionally substituted heterocyclyl (e.g., phthalimide or morpholine) each R⁸ and R⁹ is independently H, a targeting ligand (e.g., GalNac), a pharmacokinetics modifier, optionally substituted C₁₋₃₀ alkyl, optionally substituted C₁₋₃₀ alkenyl, or optionally substituted C₁₋₃₀alkynyl; and
R^{33M} is hydrogen, hydroxyl, protected hydroxyl, phosphate group, a reactive phosphorous group, optionally substituted C₁₋₃₀ alkyl, optionally substituted C₂₋₃₀alkenyl, optionally substituted C₂₋₃₀alkynyl, optionally substituted C₁₋₃₀ alkoxy (e.g., methoxy), alkoxyalkyl (e.g., methoxyethyl), alkoxyalkylamine, alkoxyoxycarboxylate, —O—C₄₋₃₀alkyl-ON(CH₂R⁸)(CH₂R⁹), —O—C₄₋₃₀alkyl-ON(CH₂R⁸)(CH₂R⁹), a solid support, a linker, a linker covalently bonded (e.g., —OC(O)CH₂CH₂C(O)—) to a solid support, a bond to an internucleotide linkage to a subsequent nucleotide, or —Z—R^{33L}.
   each Z is independently absent, a bond, O, S, or NR^{NR6};
      each R^{NR6} is independently H, optionally substituted C₁₋₃₀ alkyl, optionally substituted C₁₋₃₀haloalkyl, optionally substituted C₂₋₃₀alkenyl, optionally substituted C₂₋₃₀alkynyl, or a nitrogen protecting group;
      each R^{33L} is a ligand, a linker covalently linked to one or more ligands, optionally substituted C₁₋₃₀ alkyl, optionally substituted C₂₋₃₀alkenyl, optionally substituted C₂₋₃₀alkynyl, or polyethylene glycol;
R^{43N} hydrogen, hydroxyl, halogen, protected hydroxyl, phosphate group, a reactive phosphorous group, optionally substituted C₁₋₃₀ alkyl, optionally substituted C₁₋₃₀haloalkyl, optionally substituted C₂₋₃₀alkenyl, optionally substituted C₂₋₃₀alkynyl, optionally substituted C₁₋₃₀ alkoxy (e.g., methoxy, 2-methoxyethoxy, dimethylaminoethoxyethyoxy, N-methylmethoxyamido), alkoxyalkyl (e.g., methoxyethyl), alkoxyalkylamine, alkoxyoxycarboxylate, amino, alkylamino, dialkylamino, —O—C₄₋₃₀alkyl-ON(CH₂R⁸)(CH₂R⁹), —O—C₄₋₃₀alkyl-ON(CH₂R⁸)(CH₂R⁹), a bond to an internucleotide linkage to a subsequent nucleotide, a 3′-oligonuclotide capping group, a ligand, a solid support, a linker, a linker covalently bonded (e.g., —OC(O)CH₂CH₂C(O)—) to a solid support, or —Z-L²-R⁶;
R^{45N} is R⁶, —Z-L²-R⁶, a bond to an internucleotide linkage to a preceding nucleotide, hydrogen, hydroxyl, protected hydroxyl, phosphate group, optionally substituted C₁₋₃₀ alkyl, optionally substituted C₁₋₃₀haloalkyl, optionally substituted C₂₋₃₀alkenyl, optionally substituted C₂₋₃₀alkynyl, optionally substituted C₁₋₃₀ alkoxy, halogen, alkoxyalkyl (e.g., methoxyethyl), alkoxyalkylamine, alkoxyoxycarboxylate, amino, alkylamino, dialkylamino, —O—C₄₋₃₀alkyl-ON(CH₂R⁸)(CH₂R⁹), —O—C₄₋₃₀alkyl-ON(CH₂R⁸)(CH₂R⁹), vinylphosphonate (VP) group, C₃₋₆cycloalkylphosphonate (e.g., cyclopropylphosphonate), monophosphate ((HO)₂(O)P—O-5′), diphosphate ((HO)₂(O)P—O—P(HO)(O)—O-5′), triphosphate ((HO)₂(O)P—O—(HO)(O)P—O—P(HO)(O)—O-5′); monothiophosphate (phosphorothioate, (HO)2(S)P—O-5′), monodithiophosphate (phosphorodithioate; (HO)(HS)(S)P—O-5′), phosphorothiolate ((HO)2(O)P—S-5′); alpha-thiotriphosphate; beta-thiotriphosphate; gamma-thiotriphosphate;
phosphoramidates ((HO)₂(O)P—NH-5′, (HO)(NH₂)(O)P—O-5′), alkylphosphonates (R(OH)(O)P—O-5′, R=alkyl, e.g., methyl, ethyl, isopropyl, propyl, etc. . . . ), alkyletherphosphonates (R(OH)(O)P—O-5′, R=alkylether, e.g., methoxymethyl (CH₂OMe), ethoxymethyl, etc. . . . ), (HO)₂(X)P—O[—(CH₂)ₐ—O—P(X)(OH)—O]_{b}-5′ or (HO)₂(X)P—O[—(CH₂)ₐ—P(X)(OH)—O]_{b}-5′ or (HO)₂(X)P—[—(CH₂)ₐ—O—P(X)(OH)—O]_{b}-5′, where X is O, S or optionally substituted alkyl, and dialkyl terminal phosphates and phosphate mimics (e.g., HO[—(CH₂)ₐ—O—P(X)(OH)—O]_{b}-5′, H₂N[—(CH₂)ₐ—O—P(X)(OH)—O]_{b}-5′, H[—(CH₂)ₐ—O—P(X)(OH)—O]_{b}-5′, Me₂N[—(CH₂)ₐ—O—P(X)(OH)—O]_{b}-5′, HO[—(CH₂)ₐ—P(X)(OH)—O]_{b}-5′, H₂N[—(CH₂)ₐ—P(X)(OH)—O]_{b}-5′, H[—(CH₂)ₐ—P(X)(OH)—O]_{b}-5′, Me₂N[—(CH₂)ₐ—P(X)(OH)—O]_{b}-5′, wherein X is O or S; and a and b are each independently 1-10); and
each R^{AO} is independently —Z-L²-R⁶, and
provided that the oligonucleotide comprises at least one R⁶ group.

In some embodiments of any one of the aspects described herein, one of R³² or R³³ is a bond to an internucleotide linkage to a subsequent nucleotide or R³⁵ is a bond to an internucleotide linkage to a preceding nucleotide.

In yet another aspect, provided herein is a double-stranded nucleic acid comprising a first strand and a second strand complementary to the first strand, and wherein at least one of the first and second strand is an oligonucleotide comprising a nucleotide of selected from Formulae IIIa-IIIg and IVa-IVg described herein.

In another aspect, provided herein is a method for inhibiting or reducing the expression of a target gene in a subject. The method comprises administering to the subject: (i) a double-stranded RNA described herein, wherein one of the strands of the dsRNA is complementary to a target gene; and/or (ii) an oligonucleotide described herein, wherein the oligonucleotide is complementary to a target gene.

### BRIEF DESCRIPTION OF THE DRAWINGS

This patent or application file contains at least one drawing executed in color. Copies of this patent or patent application publication with color drawing (s) will be provided by the Office upon request and payment of the necessary fee.

FIG. 1 is a schematic representation of some exemplary embodiments of the disclosure.

FIGS. 2A-6B depict exemplary nucleotide monomers according to embodiments of the disclosure.

FIGS. 6C and 6D depict exemplary nucleotide monomers according to embodiments of the disclosure as incorporated into nucleic acids.

FIGS. 7-10 depict exemplary non-nucleotide monomers based on prolinol scaffolds (FIG. 7), serinol scaffolds (FIG. 8), D- and L-Threoninol scaffolds (FIG. 9), and conjugates derived from Pentaerythritol and Norbornyl scaffolds (FIG. 10) according to embodiments of the disclosure.

FIG. 11 depicts exemplary ligands with aldehyde and ketone linkers.

FIGS. 12-14 are schematic representations of exemplary modification sites in double-stranded nuclei acids.

FIGS. 15A-15F show results of post-synthetic conjugation of homo-dT10 and dT20 sequences.

FIG. 16 depicts synthesis of AOCC modified oligonucleotides from AOCC-conjugate building blocks according to an embodiment of the disclosure.

FIGS. 17A-17D show analysis of oligonucleotide conjugated according to embodiments of the disclosure.

FIG. 18 is a schematic representation of some exemplary aspects of the disclosure.

FIG. 19 depicts some exemplary AOCC building blocks according to some embodiments of the disclosure.

FIG. 20 is a schematic representation of some exemplary aspects of the disclosure.

FIGS. 21-24 depict exemplary building blocks according to embodiments of the disclosure.

FIG. 25 is a scheme showing oligonucleotides synthesis from AOCC building blocks.

FIG. 26 depicts post-synthetic conjugation of oligonucleotides.

FIG. 27 shows LCMS analysis of crude conjugates prepared from poly-dT sequence.

FIG. 28 depicts post-synthetic conjugation of oligonucleotides on solid support.

FIG. 29 shows LCMS analysis of crude conjugates from post-synthetic conjugation of oligonucleotides on solid support.

FIG. 30 depicts some exemplary peptides for AOCC chemistry.

FIG. 31 depicts some exemplary non-nucleotide scaffolds for AOCC.

FIG. 32 depicts some exemplary carbonyl and acid compounds for AOCC modifications.

FIG. 33 depicts some exemplary compounds comprising a 2′- or 3′-modified sugar according to some embodiments of the disclosure.

FIG. 34 depicts some exemplary compounds comprising a C4− or C5-substituted pyrimidine according to some embodiments of the disclosure.

FIG. 35 depicts some exemplary compounds comprising a N6 or C8-substituted pyrimidine according to some embodiments of the disclosure.

FIGS. 36A and 36B depict some exemplary non-nucleoside scaffolds for multivalent AOCC according to some embodiments of the disclosure.

FIGS. 37A and 37B depict structures of some abbreviations used in the nucleic acid sequences disclosed herein.

FIGS. 38A and 38B depict some exemplary embodiments of the disclosure.

### DETAILED DESCRIPTION

It is to be understood that both the foregoing general description and the following detailed description are exemplary and explanatory only and are not restrictive of the invention, as claimed. Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting. Also, terms such as “element” or “component” encompass both elements and components comprising one unit and elements and components that comprise more than one subunit, unless specifically stated otherwise.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated by reference in their entirety for any purpose.

In one aspect, provided herein is a compound of selected from formulae Ia-Ig and Ii.

R¹ (nucleobase)

In compounds of formulae Ia-Ig and Ii and nucleotides of formulae IIIa-IIIg, R¹ can be a nucleobase or a nucleobase comprising a -L¹-R⁶ group.

It is noted that the nucleobase can be a natural nucleobase or a non-natural nucleobase. By a “non-natural nucleobase” is meant a nucleobase other than adenine, guanine, cytosine, uracil, or thymine. Exemplary non-natural nucleobases include, but are not limited to, inosine, xanthine, hypoxanthine, nebularine, isoguanosine, tubercidin, and substituted or modified analogs of adenine, guanine, cytosine and uracil, such as 2-aminoadenine and other alkyl derivatives of adenine and guanine, 2-propyl and other alkyl derivatives of adenine and guanine, 5-halouracil and cytosine, 5-propynyl uracil and cytosine, 6-azo uracil, cytosine and thymine, 5-uracil (pseudouracil), 4-thiouracil, 5-halouracil, 5-(2-aminopropyl)uracil, 5-amino allyl uracil, 8-halo, amino, thiol, thioalkyl, hydroxyl and other 8-substituted adenines and guanines, 5-trifluoromethyl and other 5-substituted uracils and cytosines, 7-methylguanine, 5-substituted pyrimidines, 6-azapyrimidines and N-2, N-6 and O-6 substituted purines, including 2-aminopropyladenine, 5-propynyluracil and 5-propynylcytosine, dihydrouracil, 3-deaza-5-azacytosine, 2-aminopurine, 5-alkyluracil, 7-alkylguanine, 5-alkyl cytosine, 7-deazaadenine, N6, N6-dimethyladenine, 2,6-diaminopurine, 5-amino-allyl-uracil, N3-methyluracil, substituted 1,2,4-triazoles, 2-pyridinone, 5-nitroindole, 3-nitropyrrole, 5-methoxyuracil, uracil-5-oxyacetic acid, 5-methoxycarbonylmethyluracil, 5-methyl-2-thiouracil, 5-methoxycarbonylmethyl-2-thiouracil, 5-methylaminomethyl-2-thiouracil, 3-(3-amino-3carboxypropyl)uracil, 3-methylcytosine, 5-methylcytosine, N⁴-acetyl cytosine, 2-thiocytosine, N6-methyladenine, N6-isopentyladenine, 2-methylthio-N6-isopentenyladenine, N-methylguanines, or O-alkylated bases. Further purines and pyrimidines include those disclosed in U.S. Pat. No. 3,687,808, those disclosed in the Concise Encyclopedia of Polymer Science and Engineering, pages 858-859, Kroschwitz, J. I., ed. John Wiley & Sons, 1990, and those disclosed by Englisch et al., Angewandte Chemie, International Edition, 1991, 30, 613, content of all which is incorporated herein by reference.

In some embodiments, the non-natural nucleobase can be selected from the group consisting of inosine, xanthine, hypoxanthine, nebularine, isoguanosine, tubercidin, 2-(halo)adenine, 2-(alkyl)adenine, 2-(propyl)adenine, 2-(amino)adenine, 2-(aminoalkyl)adenine, 2-(aminopropyl)adenine, 2-(methylthio)-N⁶-(isopentenyl)adenine, 7-(deaza)adenine, 8-(alkenyl)adenine, 8-(alkyl)adenine, 8-(alkynyl)adenine, 8-(amino)adenine, 8-(halo)adenine, 8-(hydroxyl)adenine, 8-(thioalkyl)adenine, 8-(thiol)adenine, N⁶-(isopentyl)adenine, N⁶-(methyl)adenine, N⁶, N⁶-(dimethyl)adenine, 2-(alkyl)guanine, 2-(propyl)guanine, 6-(alkyl)guanine, 6-(methyl)guanine, 7-(alkyl)guanine, 7-(methyl)guanine, 7-(deaza)guanine, 8-(alkyl)guanine, 8-(alkenyl)guanine, 8-(alkynyl)guanine, 8-(amino)guanine, 8-(halo)guanine, 8-(hydroxyl)guanine, 8-(thioalkyl)guanine, 8-(thiol)guanine, N-(methyl)guanine, 2-(thio)cytosine, 3-(deaza)-5-(aza)cytosine, 3-(alkyl)cytosine, 3-(methyl)cytosine, 5-(alkyl)cytosine, 5-(alkynyl)cytosine, 5-(halo)cytosine, 5-(methyl)cytosine, 5-(propynyl)cytosine, 5-(propynyl)cytosine, 5-(trifluoromethyl)cytosine, 6-(azo)cytosine, N⁴-(acetyl)cytosine, 3-(3-amino-3-carboxypropyl)uracil, 2-(thio)uracil, 5-(methyl)-2-(thio)uracil, 5-(methylaminomethyl)-2-(thio)uracil, 4-(thio)uracil, 5-(methyl)-4-(thio)uracil, 5-(methylaminomethyl)-4-(thio)uracil, 5-(methyl)-2,4-(dithio)uracil, 5-(methylaminomethyl)-2,4-(dithio)uracil, 5-(2-aminopropyl)uracil, 5-(alkyl)uracil, 5-(alkynyl)uracil, 5-(allylamino)uracil, 5-(aminoallyl)uracil, 5-(aminoalkyl)uracil, 5-(guanidiniumalkyl)uracil, 5-(1,3-diazole-1-alkyl)uracil, 5-(cyanoalkyl)uracil, 5-(dialkylaminoalkyl)uracil, 5-(dimethylaminoalkyl)uracil, 5-(halo)uracil, 5-(methoxy)uracil, uracil-5-oxyacetic acid, 5-(methoxycarbonylmethyl)-2-(thio)uracil, 5-(methoxycarbonyl-methyl)uracil, 5-(propynyl)uracil, 5-(propynyl)uracil, 5-(trifluoromethyl)uracil, 6-(azo)uracil, dihydrouracil, N³-(methyl)uracil, 5-uracil (i.e., pseudouracil), 2-(thio)pseudouracil, 4-(thio)pseudouracil, 2,4-(dithio)pseudouracil, 5-(alkyl)pseudouracil, 5-(methyl)pseudouracil, 5-(alkyl)-2-(thio)pseudouracil, 5-(methyl)-2-(thio)pseudouracil, 5-(alkyl)-4-(thio)pseudouracil, 5-(methyl)-4-(thio)pseudouracil, 5-(alkyl)-2,4-(dithio)pseudouracil, 5-(methyl)-2,4-(dithio)pseudouracil, 1-substituted pseudouracil, 1-substituted 2(thio)-pseudouracil, 1-substituted 4-(thio)pseudouracil, 1-substituted 2,4-(dithio)pseudouracil, 1-(aminocarbonylethylenyl)-pseudouracil, 1-(aminocarbonylethylenyl)-2(thio)-pseudouracil, 1-(aminocarbonylethylenyl)-4-(thio)pseudouracil, 1-(aminocarbonylethylenyl)-2,4-(dithio)pseudouracil, 1-(aminoalkylaminocarbonylethylenyl)-pseudouracil, 1-(aminoalkylamino-carbonylethylenyl)-2(thio)-pseudouracil, 1-(aminoalkylaminocarbonylethylenyl)-4-(thio)pseudouracil, 1-(aminoalkylaminocarbonylethylenyl)-2,4-(dithio)pseudouracil, 1,3-(diaza)-2-(oxo)-phenoxazin-1-yl, 1-(aza)-2-(thio)-3-(aza)-phenoxazin-1-yl, 1,3-(diaza)-2-(oxo)-phenthiazin-1-yl, 1-(aza)-2-(thio)-3-(aza)-phenthiazin-1-yl, 7-substituted 1,3-(diaza)-2-(oxo)-phenoxazin-1-yl, 7-substituted 1-(aza)-2-(thio)-3-(aza)-phenoxazin-1-yl, 7-substituted 1,3-(diaza)-2-(oxo)-phenthiazin-1-yl, 7-substituted 1-(aza)-2-(thio)-3-(aza)-phenthiazin-1-yl, 7-(aminoalkylhydroxyl)-1,3-(diaza)-2-(oxo)-phenoxazin-1-yl, 7-(aminoalkylhydroxyl)-1-(aza)-2-(thio)-3-(aza)-phenoxazin-1-yl, 7-(aminoalkylhydroxyl)-1,3-(diaza)-2-(oxo)-phenthiazin-1-yl, 7-(aminoalkylhydroxyl)-1-(aza)-2-(thio)-3-(aza)-phenthiazin-1-yl, 7-(guanidiniumalkylhydroxyl)-1,3-(diaza)-2-(oxo)-phenoxazin-1-yl, 7-(guanidiniumalkylhydroxyl)-1-(aza)-2-(thio)-3-(aza)-phenoxazin-1-yl, 7-(guanidiniumalkyl-hydroxyl)-1,3-(diaza)-2-(oxo)-phenthiazin-1-yl, 7-(guanidiniumalkylhydroxyl)-1-(aza)-2-(thio)-3-(aza)-phenthiazin-1-yl, 1,3,5-(triaza)-2,6-(dioxa)-naphthalene, inosine, xanthine, hypoxanthine, nebularine, tubercidin, isoguanosine, inosinyl, 2-aza-inosinyl, 7-deaza-inosinyl, nitroimidazolyl, nitropyrazolyl, nitrobenzimidazolyl, nitroindazolyl, aminoindolyl, pyrrolopyrimidinyl, 3-(methyl)isocarbostyrilyl, 5-(methyl)isocarbostyrilyl, 3-(methyl)-7-(propynyl)isocarbostyrilyl, 7-(aza)indolyl, 6-(methyl)-7-(aza)indolyl, imidizopyridinyl, 9-(methyl)-imidizopyridinyl, pyrrolopyrizinyl, isocarbostyrilyl, 7-(propynyl)isocarbostyrilyl, propynyl-7-(aza)indolyl, 2,4,5-(trimethyl)phenyl, 4-(methyl)indolyl, 4,6-(dimethyl)indolyl, phenyl, napthalenyl, anthracenyl, phenanthracenyl, pyrenyl, stilbenyl, tetracenyl, pentacenyl, difluorotolyl, 4-(fluoro)-6-(methyl)benzimidazole, 4-(methyl)benzimidazole, 6-(azo)thymine, 2-pyridinone, 5-nitroindole, 3-nitropyrrole, 6-(aza)pyrimidine, 2-(amino)purine, 2,6-(diamino)purine, 5-substituted pyrimidines, N²-substituted purines, N⁶-substituted purines, O⁶-substituted purines, substituted 1,2,4-triazoles, and any O-alkylated or N-alkylated derivatives thereof.

In some embodiments, a non-natural nucleobase is a modified nucleobase, i.e., the nucleobase comprises a nucleobase modification described herein, e.g., the nucleobase is a substituted or modified analog of any of the natural nucleobases. Examples of the nucleobase modifications include, but not limited to: C-5 pyrimidine with an alkyl group or aminoalkyls and other cationic groups such as guanidinium and amidine functionalities, N²- and N⁶—with an alkyl group or aminoalkyls and other cationic groups such as guanidinium and amidine functionalities of purines, G-clamps, guanidinium G-clamps, and pseudouridine known in the art.

In some embodiments of any one of the aspects, the non-natural nucleobase is a universal nucleobase. As used herein, a universal nucleobase is any modified or unmodified natural or non-natural nucleobase that can base pair with all of adenine, cytosine, guanine and uracil without substantially affecting the melting behavior, recognition by intracellular enzymes or activity of the oligonucleotide comprising the universal nucleobase. Some exemplary universal nucleobases include, but are not limited to, 2,4-difluorotoluene, nitropyrrolyl, nitroindolyl, 8-aza-7-deazaadenine, 4-fluoro-6-methylbenzimidazle, 4-methylbenzimidazle, 3-methyl isocarbostyrilyl, 5-methyl isocarbostyrilyl, 3-methyl-7-propynyl isocarbostyrilyl, 7-azaindolyl, 6-methyl-7-azaindolyl, imidizopyridinyl, 9-methyl-imidizopyridinyl, pyrrolopyrizinyl, isocarbostyrilyl, 7-propynyl isocarbostyrilyl, propynyl-7-azaindolyl, 2,4,5-trimethylphenyl, 4-methylinolyl, 4,6-dimethylindolyl, phenyl, napthalenyl, anthracenyl, phenanthracenyl, pyrenyl, stilbenyl, tetracenyl, pentacenyl, and structural derivatives thereof.

In some embodiments of any one of the aspects described herein, the non-natural nucleobase is a protected nucleobase. As used herein, a “protected nucleobase” refers to a nucleobase comprising a nitrogen protecting group, and/or an oxygen protecting group, and/or a sulfur protecting group.

In some embodiments of any one of the aspects described herein, the non-natural nucleobase is a modified, protected or substituted analogs of a nucleobase selected from adenine, cytosine, guanine, thymine, and uracil.

In some embodiments of any one of the aspects described herein, R¹ is a pyrimidine nucleobase comprising -L¹-R⁶. For example, R¹ is a pyrimidine nucleobase comprising -L¹-R⁶ at the C₅ position. In some embodiments, R¹ is uracil substituted with -L¹-R⁶ at the C₅ position. In some embodiments, R¹ is cytosine substituted with -L¹-R⁶ at the C₅ position.

In some embodiments of any one of the aspects, R¹ is 4-aminopyrimidine nucleobase comprising -L¹-R⁶ linked to the 4-amino group. For example, R¹ is cytidine comprising -L¹-R⁶ linked to the amino at the C₄ position.

In some embodiments of any one of the aspects described herein, R¹ is a purine nucleobase comprising -L¹-R⁶. For example, R¹ is a purine nucleobase comprising -L¹-R⁶ at the C2, N6, or C8 position. In some embodiments, R¹ is adenine substituted with -L¹-R⁶ at one of C2, N6, or C8 position.

In some embodiments of any one of the aspects described herein, R¹ is a 2-amino purine nucleobase comprising -L¹-R⁶. For example, R¹ is a 2-aminopurine nucleobase comprising -L¹-R⁶ at the N2, N6, or C8 position. In some embodiments, R¹ is guanine substituted with -L¹-R⁶ at one of N2, N6, or C8 position.

In some embodiments of any one of the aspects described herein, R¹ is a N7-deaza purine nucleobase comprising -L¹-R⁶. For example, R¹ is a N7-deaza purine nucleobase comprising -L¹-R⁶ at the C2, N6, C8 or N7-deaza position. In some embodiments, R¹ is a N7-deazaadenine substituted with -L¹-R⁶ at one of C2, N6, C8 or N7-deaza position.

In some embodiments of any one of the aspects described herein, R¹ is a 2-amino-N7-deaza purine nucleobase comprising -L¹-R⁶. For example, R¹ is a 2-amino-N7-deaza purine nucleobase comprising -L¹-R⁶ at the N2, N6, C8 or N7-deaza position. In some embodiments, R¹ is a N7-deazaguanine substituted with -L¹-R⁶ at one of C2, N6, C8 or N7-deaza position.

### L¹

In some embodiments of the various aspects described herein, L¹ is a linker.

As used herein, the term “linker” means an organic moiety that connects two parts of a compound. Linkers typically comprise a direct bond or an atom such as oxygen or sulfur, a unit such as NR^{N1}, C(O), C(O)O, C(O)NR¹, SO, SO₂, SO₂NH or a chain of atoms, such as substituted or unsubstituted alkyl, substituted or unsubstituted alkenyl, substituted or unsubstituted alkynyl, arylalkyl, arylalkenyl, arylalkynyl, heteroarylalkyl, heteroarylalkenyl, heteroarylalkynyl, heterocyclylalkyl, heterocyclylalkenyl, heterocyclylalkynyl, aryl, heteroaryl, heterocyclyl, cycloalkyl, cycloalkenyl, alkylarylalkyl, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkylheteroarylalkenyl, alkylheteroarylalkynyl, alkenylheteroarylalkyl, alkenylheteroarylalkenyl, alkenylheteroarylalkynyl, alkynylheteroarylalkyl, alkynylheteroarylalkenyl, alkynylheteroarylalkynyl, alkylheterocyclylalkyl, alkylheterocyclylalkenyl, alkylhererocyclylalkynyl, alkenylheterocyclylalkyl, alkenylheterocyclylalkenyl, alkenylheterocyclylalkynyl, alkynylheterocyclylalkyl, alkynylheterocyclylalkenyl, alkynylheterocyclylalkynyl, alkylaryl, alkenylaryl, alkynylaryl, alkylheteroaryl, alkenylheteroaryl, alkynylhereroaryl, where one or more methylenes can be interrupted or terminated by O, S, S(O), SO₂, NR¹, NR¹—C(O), C(O), C(O)O, cleavable linking group, substituted or unsubstituted aryl, substituted or unsubstituted heteroaryl, substituted or unsubstituted heterocyclic; where R^{N1} is hydrogen, acyl, aliphatic or substituted aliphatic.

In some embodiments, the linker is a cleavable linker. Cleavable linkers are those that rely on processes inside a target cell to liberate the two parts the linker is holding together, as reduction in the cytoplasm, exposure to acidic conditions in a lysosome or endosome, or cleavage by specific enzymes (e.g. proteases) within the cell. As such, cleavable linkers allow the two parts to be released in their original form after internalization and processing inside a target cell. Cleavable linkers include, but are not limited to, those whose bonds can be cleaved by enzymes (e.g., peptide linkers); reducing conditions (e.g., disulfide linkers); or acidic conditions (e.g., hydrazones and carbonates).

Generally, the cleavable linker comprises at least one cleavable linking group. A cleavable linking group is one which is sufficiently stable outside the cell, but which upon entry into a target cell is cleaved to release the two parts the linker is holding together. In a preferred embodiment, the cleavable linking group is cleaved at least 10 times or more, preferably at least 100 times faster in the target cell or under a first reference condition (which can, e.g., be selected to mimic or represent intracellular conditions) than in the blood or serum of a subject, or under a second reference condition (which can, e.g., be selected to mimic or represent conditions found in the blood or serum).

Cleavable linking groups are susceptible to cleavage agents, e.g., pH, redox potential or the presence of degradative molecules. Generally, cleavage agents are more prevalent or found at higher levels or activities inside cells than in serum or blood. Examples of such degradative agents include: redox agents which are selected for particular substrates or which have no substrate specificity, including, e.g., oxidative or reductive enzymes or reductive agents such as mercaptans, present in cells, that can degrade a redox cleavable linking group by reduction; esterases; endosomes or agents that can create an acidic environment, e.g., those that result in a pH of five or lower; enzymes that can hydrolyze or degrade an acid cleavable linking group by acting as a general acid, peptidases (which can be substrate specific), and phosphatases.

A cleavable linkage group, such as a disulfide bond can be susceptible to pH. The pH of human serum is 7.4, while the average intracellular pH is slightly lower, ranging from about 7.1-7.3. Endosomes have a more acidic pH, in the range of 5.5-6.0, and lysosomes have an even more acidic pH at around 5.0. Some linkers will have a cleavable linking group that is cleaved at a preferred pH, thereby releasing the cationic lipid from the ligand inside the cell, or into the desired compartment of the cell.

A linker can include a cleavable linking group that is cleavable by a particular enzyme. The type of cleavable linking group incorporated into a linker can depend on the cell to be targeted. For example, liver targeting ligands can be linked to the cationic lipids through a linker that includes an ester group. Liver cells are rich in esterases, and therefore the linker will be cleaved more efficiently in liver cells than in cell types that are not esterase-rich. Other cell-types rich in esterases include cells of the lung, renal cortex, and testis. Linkers that contain peptide bonds can be used when targeting cell types rich in peptidases, such as liver cells and synoviocytes.

In general, the suitability of a candidate cleavable linking group can be evaluated by testing the ability of a degradative agent (or condition) to cleave the candidate linking group. It will also be desirable to also test the candidate cleavable linking group for the ability to resist cleavage in the blood or when in contact with other non-target tissue. Thus one can determine the relative susceptibility to cleavage between a first and a second condition, where the first is selected to be indicative of cleavage in a target cell and the second is selected to be indicative of cleavage in other tissues or biological fluids, e.g., blood or serum. The evaluations can be carried out in cell free systems, in cells, in cell culture, in organ or tissue culture, or in whole animals. It may be useful to make initial evaluations in cell-free or culture conditions and to confirm by further evaluations in whole animals. In preferred embodiments, useful candidate compounds are cleaved at least 2, 4, 10 or 100 times faster in the cell (or under in vitro conditions selected to mimic intracellular conditions) as compared to blood or serum (or under in vitro conditions selected to mimic extracellular conditions).

One class of cleavable linking groups is redox cleavable linking groups, which may be used in the dsRNA molecule according to the present invention that are cleaved upon reduction or oxidation. An example of reductively cleavable linking group is a disulfide linking group (—S—S—). To determine if a candidate cleavable linking group is a suitable “reductively cleavable linking group,” or for example is suitable for use with a particular iRNA moiety and particular targeting agent one can look to methods described herein. For example, a candidate can be evaluated by incubation with dithiothreitol (DTT), or other reducing agent using reagents know in the art, which mimic the rate of cleavage which would be observed in a cell, e.g., a target cell. The candidates can also be evaluated under conditions which are selected to mimic blood or serum conditions. In a preferred embodiment, candidate compounds are cleaved by at most 10% in the blood. In preferred embodiments, useful candidate compounds are degraded at least 2, 4, 10 or 100 times faster in the cell (or under in vitro conditions selected to mimic intracellular conditions) as compared to blood (or under in vitro conditions selected to mimic extracellular conditions). The rate of cleavage of candidate compounds can be determined using standard enzyme kinetics assays under conditions chosen to mimic intracellular media and compared to conditions chosen to mimic extracellular media.

Phosphate-based cleavable linking groups, which may be used in the dsRNA molecule according to the present invention, are cleaved by agents that degrade or hydrolyze the phosphate group. An example of an agent that cleaves phosphate groups in cells are enzymes such as phosphatases in cells. Examples of phosphate-based linking groups are —O—P(O)(ORk)-O—, —O—P(S)(ORk)-O—, —O—P(S)(SRk)-O—, —S—P(O)(ORk)-O—, —O—P(O)(ORk)-S—, —S—P(O)(ORk)-S—, —O—P(S)(ORk)-S—, —S—P(S)(ORk)-O—, —O—P(O)(Rk)-O—, —O—P(S)(Rk)-O—, —S—P(O)(Rk)-O—, —S—P(S)(Rk)-O—, —S—P(O)(Rk)-S—, —O—P(S)(Rk)-S—, wherein Rk at each occurrence can be, independently, hydrogen, C₁-C₂₀ alkyl, C₁-C₂₀ haloalkyl, C₆-C₁₀ aryl, C₇-C₁₂ aralkyl. Preferred embodiments are —O—P(O)(OH)—O—, —O—P(S)(OH)—O—, —O—P(S)(SH)—O—, —S—P(O)(OH)—O—, —O—P(O)(OH)—S—, —S—P(O)(OH)—S—, —O—P(S)(OH)—S—, —S—P(S)(OH)—O—, —O—P(O)(H)—O—, —O—P(S)(H)—O—, —S—P(O)(H)—O—, —S—P(S)(H)—O—, —S—P(O)(H)—S—, —O—P(S)(H)—S—. A preferred embodiment is —O—P(O)(OH)—O—. These candidates can be evaluated using methods analogous to those described above.

Acid cleavable linking groups, which may be used in the dsRNA molecule according to the present invention, are linking groups that are cleaved under acidic conditions. In preferred embodiments acid cleavable linking groups are cleaved in an acidic environment with a pH of about 6.5 or lower (e.g., about 6.0, 5.5, 5.0, or lower), or by agents such as enzymes that can act as a general acid. In a cell, specific low pH organelles, such as endosomes and lysosomes can provide a cleaving environment for acid cleavable linking groups. Examples of acid cleavable linking groups include but are not limited to hydrazones, esters, and esters of amino acids. Acid cleavable groups can have the general formula —C═NN—, C(O)O, or —OC(O). A preferred embodiment is when the carbon attached to the oxygen of the ester (the alkoxy group) is an aryl group, substituted alkyl group, or tertiary alkyl group such as dimethyl pentyl or t-butyl. These candidates can be evaluated using methods analogous to those described above.

Ester-based cleavable linking groups, which may be used in the dsRNA molecule according to the present invention, are cleaved by enzymes such as esterases and amidases in cells. Examples of ester-based cleavable linking groups include but are not limited to esters of alkylene, alkenylene and alkynylene groups. Ester cleavable linking groups have the general formula —C(O)O—, or —OC(O)—. These candidates can be evaluated using methods analogous to those described above.

Peptide-based cleavable linking groups, which may be used in the dsRNA molecule according to the present invention, are cleaved by enzymes such as peptidases and proteases in cells. Peptide-based cleavable linking groups are peptide bonds formed between amino acids to yield oligopeptides (e.g., dipeptides, tripeptides etc.) and polypeptides. Peptide-based cleavable groups do not include the amide group (—C(O)NH—). The amide group can be formed between any alkylene, alkenylene or alkynylene. A peptide bond is a special type of amide bond formed between amino acids to yield peptides and proteins. The peptide based cleavage group is generally limited to the peptide bond (i.e., the amide bond) formed between amino acids yielding peptides and proteins and does not include the entire amide functional group. Peptide-based cleavable linking groups have the general formula —NHCHR^{A}C(O)NHCHR^{B}C(O)—, where R^{A} and R^{B} are the R groups of the two adjacent amino acids.

In some embodiments of any one of the aspects described herein, L¹ is a bond. optionally substituted C₁-C₂₀alkylene, optionally substituted C₂-C₂₀alkenylene or optionally substituted C₂-C₂₀alkynylene, and where the backbone of the alkylene, alkenylene or alkynylene can be interrupted or terminated by O, S, S(O), SO₂, NR¹, NR¹—C(O), C(O), C(O)O, cleavable linking group, substituted or unsubstituted aryl, substituted or unsubstituted heteroaryl, substituted or unsubstituted heterocyclic; where R^{N1} is hydrogen, acyl, aliphatic or substituted aliphatic.

In some embodiments of any one of the aspects described herein, L¹ is an optionally substituted C₁-C₂₀alkylene, (e.g., —(CH₂)_{b}—, where b is 1, 2, 3, 4, 5, 6, 7, 8, 9, 10, 11, 12, 13, 14, 14, 15, 16, 17, 18, 19 or 20), or optionally substituted C₂-C₂₀alkynylene, and where the backbone of the alkylene or alkynylene can be interrupted or terminated by O, S, S(O), SO₂, NR¹, NR¹—C(O), C(O), C(O)O, cleavable linking group, substituted or unsubstituted aryl, substituted or unsubstituted heteroaryl, substituted or unsubstituted heterocyclic; where R^{N1} is hydrogen, acyl, aliphatic or substituted aliphatic. For example, L¹ is an optionally substituted C₁₀-C₁₄alkylene or an optionally substituted C₂-C₆alkynylene. In some embodiments of any one of the aspects described herein, L¹ is an optionally substituted C₂alkylene, C₁₂alkylene or C₃alkynylene.

In some embodiments, L¹ is an optionally substitute C₂-C₁₆alkylene.

In some embodiments, L¹ is —C(O)NH-aliphatic or -aliphatic-C(O)NH-aliphatic. For example, L¹ is —(CH₂)ₙ₁—C(O)NH—(CH₂)ₙ₁—CH₂— or —CH₂CH₂C(O)NH—(CH₂)ₙ₁—CH₂—, where each n1 is independently 0-16. For example, L¹ is —C(O)NH—(CH₂)ₙ₁—CH₂—. In some embodiments, L¹ is —CH₂CH₂C(O)NH—(CH₂)ₙ₁—CH₂—. In some embodiments, L¹ is —CH═CHC(O)NH—(CH₂)ₙ₁—CH₂—

In some embodiments of any one of the aspects, L¹ is a bond.

In some embodiments of any one of the aspects, L¹ is absent.

### Z

In the various aspects described herein, each Z is independently absent, a bond, O, S, or NR^{NR6}, where R^{NR6} is independently H, optionally substituted C₁₋₃₀ alkyl, optionally substituted C₂₋₃₀alkenyl, optionally substituted C₂₋₃₀alkynyl, optionally substituted aryl, optionally substituted heteroaryl, optionally substituted cycloalkyl, optionally substituted heterocyclyl, or a nitrogen protecting group.

In some embodiments of the various aspects described herein, Z is O.

In some embodiments of the various aspects described herein, Z is S.

In some embodiments of the various aspects described herein, Z is NR^{NR6}.

In some embodiments of the various aspects described herein, Z is absent.

### R²

In some embodiments of any one of the aspects described herein, R² is —Z-L²-R⁶, hydrogen, halogen, —OR³²², —SR³²³, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, amino (NH₂), alkylamino, dialkylamino, heterocyclyl, arylamino, diarylamino, heteroarylamino, diheteroarylamino, amino acid, —O(CH₂CH₂O)ᵣCH₂CH₂OR³²⁴, cyano, alkyl-thio-alkyl, thioalkoxy, cycloalkyl, aryl, heteroaryl, —NH(CH₂CH₂NH)ₛCH₂CH₂—R³²⁵, NHC(O)R³²⁶, a lipid, a linker covalently attached to a lipid, a ligand, a linker covalently attached to a ligand, a solid support, a linker covalently attached to a solid support, or a reactive phosphorus group.

R³²² can be H, hydroxyl protecting group, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, cycloalkyl, heterocyclyl, aryl, heteroaryl. R³²³ can be H, sulfur protecting group, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, cycloalkyl, heterocyclyl, aryl, heteroaryl. R³²⁴ can be H, hydroxyl protecting group, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, cycloalkyl, heterocyclyl, aryl, heteroaryl. R³²⁵ can be hydrogen, halogen, hydroxyl, protected hydroxyl, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, amino (NH₂), alkylamino, dialkylamino, heterocyclyl, arylamino, diarylamino, heteroarylamino, diheteroarylamino, amino acid, cyano, alkyl-thio-alkyl, thioalkoxy, cycloalkyl, aryl, or heteroaryl. R³²⁶ can be can be hydrogen, halogen, hydroxyl, protected hydroxyl, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, amino (NH₂), alkylamino, dialkylamino, heterocyclyl, arylamino, diarylamino, heteroarylamino, diheteroarylamino, amino acid, cyano, alkyl-thio-alkyl, thioalkoxy, cycloalkyl, aryl, or heteroaryl.

In some embodiments of any one of the aspects described herein, R² is —Z-L²-R⁶, hydrogen, halogen, —OR³²², —SR³²³, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, amino (NH₂), alkylamino, dialkylamino, heterocyclyl, arylamino, diarylamino, heteroarylamino, diheteroarylamino, amino acid, —O(CH₂CH₂O)ᵣCH₂CH₂OR³²⁴, cyano, alkyl-thio-alkyl, thioalkoxy, cycloalkyl, aryl, heteroaryl, —NH(CH₂CH₂NH)ₛCH₂CH₂—R³²⁵, NHC(O)R³²⁴.

In some embodiments of any one of the aspects described herein, R² is —Z-L²-R⁶, hydrogen, hydroxyl, protected hydroxyl, halogen, optionally substituted C₁₋₃₀ alkyl, optionally substituted C₂₋₃₀alkenyl, optionally substituted C₂₋₃₀alkynyl, optionally substituted C₁₋₃₀ alkoxy, alkoxyalkyl (e.g., methoxy, 2-methoxyethoxy, dimethylaminoethoxyethyoxy, N-methylmethoxyamido), alkoxyalkylamine, alkoxyoxycarboxylate, amino, alkylamino, dialkylamino, —O—C₄₋₃₀alkyl-ON(CH₂R⁸)(CH₂R⁹), or —O—C₄₋₃₀alkyl-ON(CH₂R⁸)(CH₂R⁹). For example, R² is hydrogen, hydroxyl, protected hydroxyl, halogen, optionally substituted C₁₋₃₀ alkoxy, alkoxyalkyl (e.g., methoxy, 2-methoxyethoxy, dimethylaminoethoxyethyoxy, N-methylmethoxyamido), alkoxyalkylamine, alkoxyoxycarboxylate, amino, alkylamino, or dialkylamino.

In some embodiments of any one of the aspect, R² is —Z-L²-R⁶, hydrogen, hydroxyl, protected hydroxyl, halogen, optionally substituted C₁₋₃₀ alkoxy, or alkoxyalkyl (e.g., methoxy, 2-methoxyethoxy, dimethylaminoethoxyethyoxy, N-methylmethoxyamido).

In some embodiments of any one of the aspects, R² is —Z-L²-R⁶, hydrogen, hydroxyl, protected hydroxyl, fluoro, methoxy, 2-methoxyethoxy, —O-dimethylaminoethoxyethyl (—O-DMAEOE) or —O—N-methylacetamido (—O-NMA).

In some embodiments of any one of the aspects, R² is —Z-L²-R⁶, hydrogen, hydroxyl, protected hydroxyl, fluoro, methoxy, 2-methoxyethoxy, —O-dimethylaminoethoxyethyl (—O-DMAEOE) or —O—N-methylacetamido (—O-NMA).

In some embodiments of any one of the aspects, R² is hydrogen, hydroxyl, protected hydroxyl, fluoro, methoxy, 2-methoxyethoxy, —O-dimethylaminoethoxyethyl (—O-DMAEOE) or —O—N-methylacetamido (—O-NMA).

In some embodiments of any one of the aspects, R² is —Z-L²-R⁶. For example, R² is —Z-L²-R⁶, where Z is O.

In some embodiments of any one of the aspects, R² is —Z-L²-R⁶. For example, R² is —Z-L²-R⁶, where L² is optionally substituted C₁-C₂₀alkylene, optionally substituted C₂-C₂₀alkenylene or optionally substituted C₂-C₂₀alkynylene, and where the backbone of the alkylene, alkenylene or alkynylene can be interrupted or terminated by O, S, S(O), SO₂, NR¹, NR¹—C(O), C(O), C(O)O, cleavable linking group, substituted or unsubstituted aryl, substituted or unsubstituted heteroaryl, substituted or unsubstituted heterocyclic; where R^{N1} is hydrogen, acyl, aliphatic or substituted aliphatic.

In some embodiments of any one of the aspects, R² is —Z-L²-R⁶, where L² is a polyethylene glycol (PEG).

In some embodiments of any one of the aspects, R² is —Z-L²-R⁶, where Z is O and L² is optionally substituted C₁-C₂₀alkylene, optionally substituted C₂-C₂₀alkenylene or optionally substituted C₂-C₂₀alkynylene, and where the backbone of the alkylene, alkenylene or alkynylene can be interrupted or terminated by O, S, S(O), SO₂, NR¹, NR¹—C(O), C(O), C(O)O, cleavable linking group, substituted or unsubstituted aryl, substituted or unsubstituted heteroaryl, substituted or unsubstituted heterocyclic; where R^{N1} is hydrogen, acyl, aliphatic or substituted aliphatic.

In some embodiments of any one of the aspects, R² is —Z-L²-R⁶, where Z is O and L² is a polyethylene glycol (PEG).

In some embodiments of any one of the aspects, R² is —Z-L²-R⁶, where R⁶ is —O—N(R⁷)R^{7′} or —O—N═C(R⁷)R^{7′}. For example, R² is —O-L²-O—N(R⁷)R^{7′} or —O-L²-O—N═C(R⁷)R⁷, where L² is optionally substituted C₁-C₂₀alkylene, optionally substituted C₂-C₂₀alkenylene or optionally substituted C₂-C₂₀alkynylene, and where the backbone of the alkylene, alkenylene or alkynylene can be interrupted or terminated by O, S, S(O), SO₂, NR¹, NR¹—C(O), C(O), C(O)O, cleavable linking group, substituted or unsubstituted aryl, substituted or unsubstituted heteroaryl, substituted or unsubstituted heterocyclic; where R^{N1} is hydrogen, acyl, aliphatic or substituted aliphatic.

In some embodiments of any one of the aspects, R² is —Z-L²-R⁶, where R⁶ is —O—N(R⁷)R^{7′}. For example, R² is —Z-L²-R⁶, where R⁶ is —O—N(R⁷)R^{7′}, where R⁷ and R^{7′} are independently H or a ligand, (e.g., a ligand selected independently from the group consisting of carbohydrates, lipids, vitamins, peptides, proteins, lipoproteins, peptidomimetics, polyamines, nucleosides and nucleotides, oligonucleotides, therapeutic agents, diagnostic agents, detectable labels, antibodies or fragments thereof, optionally substituted C₁₋₃₀ alkyl, optionally substituted C₁₋₃₀ alkenyl, optionally substituted C₁₋₃₀alkynyl, optionally substituted cycloalkyl, optionally substituted heterocyclyl, optionally substituted aryl, optionally substituted heteroaryl, polyethylene glycols (PEGs), nitrogen protecting group.

In some embodiments of any one of the aspects, R² is —Z-L²-R⁶, where R⁶ is —O—N(R⁷)R^{7′}, where R⁷ and R^{7′} together with the N they are attached to form an optionally substituted heterocyclyl (e.g., phthalimide or morpholine).

In some embodiments of any one of the aspects, R² is —Z-L²-R⁶, where R⁶ is —O—N═C(R⁷)R⁷.

In some embodiments of any one of the aspects described herein, R² and R⁴ taken together are 4′-C(R¹⁰R¹¹)ᵥ—Y-2′ or 4′-Y—C(R¹⁰R¹¹)ᵥ-2′; v is 1, 2 or 3; where Y is —O—, —CH₂—, —CH(Me)-, —C(CH₃)₂—, —S—, —N(R¹²)—, —C(O)—, —C(S)—, —S(O)—, —S(O)₂—, —OC(O)—, —C(O)O—, —N(R¹²)C(O)—, or —C(O)N(R¹²)—; R¹⁰ and R¹¹ independently are H, optionally substituted C₁-C₆alkyl, optionally substituted C₂-C₆alkenyl or optionally substituted C₂-C₆alkynyl; R¹² is hydrogen, optionally substituted C₁₋₃₀alkyl, optionally substituted C₁-C₃₀alkoxy, C₁₋₄haloalkyl, optionally substituted C₂₋₄alkenyl, optionally substituted C₂₋₄alkynyl, optionally substituted C₁₋₃₀alky-CO₂H, or a nitrogen-protecting group.

In some embodiments of any one of the aspects, v is 1. In some other embodiments of any one of the aspects, v is 2. In some embodiments, Y is O.

In some embodiments, R² and R⁴ taken together are 4′-C(R¹⁰R¹¹)ᵥ—O-2′.

It is noted that R¹⁰ and R¹¹ attached to the same carbon can be same or different. For example, one of R¹⁰ and R¹¹ can be H and the other of the R¹⁰ and R¹¹ can be an optionally substituted C₁-C₆alkyl. In one non-limiting example, one of R¹⁰ and R¹¹ can be H and the other can be C₁-C₆alkyl, optionally substituted with 1, 2, 3, 4 or 5 substituents independently selected from OH, CN, SC(O)Ph, oxo (=O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]2, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)— alkyl, C(O)— alkyl, alkylcarbonylaminyl, CH₂—[CH(OH)]m-(CH₂)ₚ—OH, CH₂—[CH(OH)]m-(CH₂)ₚ—NH₂ or CH₂-aryl-alkoxy, where “m” and “p” are independently 1, 2, 3, 4, 5 or 6. For example, R¹⁰ and R¹¹ independently are H or C₁-C₃₀alkyl optionally substituted with a NH₂, OH, C(O)NH₂, COOH, halo, SH, or C₁-C₆alkoxy. In some embodiments of any one of the aspects, one of R¹⁰ and R¹¹ is H and the other is C₁-C₆alkyl, optionally substituted with a C₁-C₆alkoxy. For example, one of R¹⁰ and R¹¹ is H and the other is —CH₃ or CH₂OCH₃. In some embodiments of any one of the aspects, R¹⁰ and R¹¹ attached to the same C are the same. For example, R¹⁰ and R¹¹ attached to the same C are H.

In some embodiments of any one of the aspects, R² and R⁴ taken together are 4′-CH₂—O-2′, 4′CH(CH₃)—O-2′, 4′-CH(CH₂OCH₃)—O-2′, or 4′-CH₂CH₂—O-2′. For example, R² and R⁴ taken together are 4′-CH₂CH₂—O-2′.

In some embodiments of any one of the aspects described herein, R² and R⁴ taken together are 4′-C(R¹⁰R¹¹)—O-2′, and where one of R¹⁰ and R¹¹ is H and other is alkyl, secondary alkyl, homo-branched alkyl, hetero-branched alkyl, alkyl carboxylic esters, alkyl amines, or alkyl ether. For example, R² and R⁴ taken together are 4′-CHR¹¹—O-2′, where R¹¹ is alkyl, secondary alkyl, homo-branched alkyl, hetero-branched alkyl, alkyl carboxylic esters, alkyl amines, or alkyl ether.

In some embodiments of any one of the aspects described herein, R² is a reactive phosphorus group.

Without wishing to be bound by a theory, reactive phosphorus groups are useful for forming internucleoside linkages including for example phosphodiester and phosphorothioate internucleoside linkages. Such reactive phosphorus groups are known in the art and contain phosphorus atoms in P^{III} or P^{V} valence state including, but not limited to, phosphoramidite, H-phosphonate, phosphate triesters and phosphorus containing chiral auxiliaries. Reactive phosphorous group in the form of phosphoramidites (P^{III} chemistry) as reactive phosphites are a preferred reactive phosphorous group for solid phase oligonucleotide synthesis. The intermediate phosphite compounds are subsequently oxidized to the Pv state using known methods to yield phosphodiester or phosphorothioate internucleoside linkages.

In some embodiments of any one of the aspects described herein, the reactive phosphorous group is —OP(OR)(N(R^{P2})₂), —OP(SR)(N(R^{P2})₂), —OP(O)(OR^{P})(N(R^{P2})₂), —OP(S)(OR^{P})(N(R^{P2})₂), —OP(O)(SR^{P})(N(R^{P2})₂), —OP(O)(OR)H, —OP(S)(OR)H, —OP(O)(SR)H, —OP(O)(OR^{P})R^{P3}, —OP(S)(OR)R^{P3}, or —OP(O)(SR^{P})R^{P3}. For example, the reactive phosphorous group is —OP(OR)(N(R^{P2})₂).

In some embodiments of any one of the aspects, R^{P} is an optionally substituted C₁₋₆alkyl. For example, R^{P} is a C₁₋₆alkyl, optionally substituted with 1, 2, 3, 4 or 5 substituents independently selected from OH, CN, SC(O)Ph, oxo (=O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]2, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)-alkyl, C(O)— alkyl, alkylcarbonylaminyl, CH₂—[CH(OH)]m-(CH₂)ₚ—OH, CH₂—[CH(OH)]m-(CH₂)ₚ—NH₂ or CH₂-aryl-alkoxy, where “m” and “p” are independently 1, 2, 3, 4, 5 or 6. In some embodiments, R^{P} is a C₁₋₆alkyl, optionally substituted with a CN or —SC(O)Ph. For example, R^{P} is cyanoethyl (—CH₂CH₂CN).

In the reactive phosphorous groups, each R^{P2} is independently optionally substituted C₁₋₆alkyl. For example, each R^{P2} can be independently selected from methyl, ethyl, propyl, isopropyl, n-butyl, iso-butyl, pentyl or hexyl. It is noted that when two or more R^{P2} groups are present in the reactive phosphorous group, they can be same or different. Thus, in some none-limiting examples, when two or more R^{P2} groups are present, the R^{P2} groups are different. In some other non-limiting examples, when two or more R^{P2} groups are present, the R^{P2} groups are same. In some embodiments of any one of the aspects, each R^{P2} is isopropyl.

In some embodiments of any one of the aspects, both R^{P2} taken together with the nitrogen atom to which they are attached form an optionally substituted 3-8 membered heterocyclyl. Exemplary heterocyclyls include, but are not limited to, pyrrolidinyl, piperazinyl, dioxanyl, morpholinyl, tetrahydrofuranyl, piperidyl, 4-morpholyl, 4-piperazinyl, pyrrolidinyl, perhydropyrrolizinyl, 1,4-diazaperhydroepinyl, 1,3-dioxanyl, 1,4-dioxanyl and the like, each of which can be optionally substituted with 1, 2 or 3 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]2, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)— alkyl, C(O)— alkyl, alkylcarbonylaminyl, CH₂—[CH(OH)]m-(CH₂)ₚ—OH, CH₂—[CH(OH)]m-(CH₂)ₚ—NH₂ or CH₂-aryl-alkoxy, where “m” and “p” are independently 1, 2, 3, 4, 5 or 6.

In some embodiments of any one of the aspects, R^{P} and one of R^{P2} taken together with the atoms to which they are attached form an optionally substituted 4-8 membered heterocyclyl. Exemplary heterocyclyls include, but are not limited to, pyrrolidinyl, piperazinyl, dioxanyl, morpholinyl, tetrahydrofuranyl, piperidyl, 4-morpholyl, 4-piperazinyl, pyrrolidinyl, perhydropyrrolizinyl, 1,4-diazaperhydroepinyl, 1,3-dioxanyl, 1,4-dioxanyl and the like, each of which can be optionally substituted with 1, 2 or 3 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]2, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)— alkyl, C(O)— alkyl, alkylcarbonylaminyl, CH₂—[CH(OH)]ₘ-(CH₂)ₚ-OH, CH₂—[CH(OH)]m-(CH₂)ₚ-NH₂ or CH₂-aryl-alkoxy, where “m” and “p” are independently 1, 2, 3, 4, 5 or 6.

In the reactive phosphorous groups, each R^{P3} is independently optionally substituted C₁₋₆alkyl. For example, R^{P3} can be a C₁₋₆alkyl, optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]2, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)— alkyl, C(O)— alkyl, alkylcarbonylaminyl, CH₂—[CH(OH)]m-(CH₂)ₚ—OH, CH₂—[CH(OH)]m-(CH₂)ₚ—NH₂ or CH₂-aryl-alkoxy, where “m” and “p” are independently 1, 2, 3, 4, 5 or 6. For example, R^{P3} is methyl, ethyl, propyl, isopropyl, n-butyl, iso-butyl, pentyl or hexyl, each of which can be optionally substituted with a NH₂, OH, C(O)NH₂, COOH, halo, SH, or C₁-C₆alkoxy.

In some embodiments of any one of the aspects, the reactive phosphorous group is —OP(OR)(N(R^{P2})₂). For example, the reactive phosphorous group is —OP(OR)(N(R^{P2})₂), where R^{P} is cyanoethyl (—CH₂CH₂CN) and each R^{P2} is isopropyl.

In some embodiments of any one of the aspects described herein, R² is —OP(OR)(N(R^{P2})₂), —OP(SR^{P})(N(R^{P2})₂), —OP(O)(OR)(N(R^{P2})₂), —OP(S)(OR^{P})(N(R^{P2})₂), —OP(O)(SR^{P})(N(R^{P2})₂), —OP(O)(OR^{P})H, —OP(S)(OR^{P})H, —OP(O)(SR^{P})H, —OP(O)(OR^{P})R^{P3}, —OP(S)(OR)R^{P3}, or —OP(O)(SR^{P})R³.

In some embodiments of any one of the aspects, R² is —OP(OR) (N(R^{P2})₂), —OP(SR^{P})(N(R^{P2})₂), —OP(O)(OR^{P})(N(R^{P2})₂), —OP(S)(OR)(N(R^{P2})₂), —OP(O)(SR^{P})(N(R^{P2})₂), —OP(O)(OR^{P})H, —OP(S)(OR^{P}) an optionally substituted C₁₋₆alkyl, each R^{P2} is independently optionally substituted C₁₋₆alkyl; and each R^{P3} is independently optionally substituted C₁₋₆alkyl.

In some embodiments of any one of the aspects, R² is —OP(OR^{P})(N(R^{P2})₂). For example, the R² is —OP(OR)(N(R^{P2})₂), where R^{P} is cyanoethyl (—CH₂CH₂CN) and each R^{P2} is isopropyl.

In some embodiments of any one of the aspects descried herein, R² is a solid support or a linker covalently attached to a solid support. For example, R² is —OC(O)CH₂CH₂C(O)NH—Z, where Z is a solid support. In some embodiments, R² is —OC(O)CH₂CH₂CO₂H.

In some embodiments of any one of the aspects, when R² is —OR³²², R³²² can be hydrogen or a hydroxyl protecting group.

When R² is —SR³²³, R³²³ can be hydrogen or a sulfur protecting group. Accordingly, in some embodiments of any one of the aspects, R³²³ is hydrogen.

When R² is —O(CH₂CH₂O)ᵣCH₂CH₂OR³²⁴, r can be 1-50; R³²⁴ is independently for each occurrence H, C₁-C₃₀alkyl, cyclyl, heterocyclyl, aryl, heteroaryl, aralkyl, sugar or R³²⁵; and R³²⁵ is independently for each occurrence amino (NH₂), alkylamino, dialkylamino, arylamino, diarylamino, heteroarylamino, or diheteroaryl amino.

When R² is —NH(CH₂CH₂NH)ₛCH₂CH₂—R³²⁵, s can be 1-50 and R³²⁵ can be independently for each occurrence amino (NH₂), alkylamino, dialkylamino, arylamino, diarylamino, heteroarylamino, or diheteroaryl amino.

In some embodiments of any one of the aspects described herein, R² is hydrogen, halogen, —OR³²², or optionally substituted C₁-C₃₀alkoxy. For example, R² is halogen, —OR³²², or optionally substituted C₁-C₃₀alkoxy. In some embodiments of any one of the aspects described herein, R² is F, OH or optionally substituted C₁-C₃₀alkoxy.

In some embodiments of any one of the aspects described herein, R² is C₁-C₃₀alkoxy optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]2, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)— alkyl, C(O)— alkyl, alkylcarbonylaminyl, CH₂—[CH(OH)]m-(CH₂)ₚ—OH, CH₂—[CH(OH)]m-(CH₂)ₚ—NH₂ or CH₂-aryl-alkoxy, where “m” and “p” are independently 1, 2, 3, 4, 5 or 6. For example, R² is C₁-C₃₀alkoxy optionally substituted with a NH₂, OH, C(O)NH₂, COOH, halo, SH, or C₁-C₆alkoxy. In some embodiments of any one of the aspects described herein, R² is —O(CH₂)ₜCH₃, where t is 1-21.

In some embodiments, t is 14, 15, 16, 17 or 18. In one non-limiting example, t is 16.

In some embodiments of any one of the aspects, R² is —O(CH₂)ᵤR³²⁷, where u is 2-10; R³²⁷ is C₁-C₆alkoxy, amino (NH₂), CO₂H, OH or halo. For example, R³²⁷ is —CH₃ or NH₂. Accordingly, in some embodiments of any one of the aspects described herein, R² is —O(CH₂)ᵤ—OMe or R² is —O(CH₂)ᵤNH₂

In some embodiments of any one of the aspects described herein, u is 2, 3, 4, 5 or 6. For example, u is 2, 3 or 6. In one non-limiting example, u is 2. In another non-limiting example, u is 3 or 6.

In some embodiments of any one of the aspects described herein, R² is a C₁-C₆haloalkyl. For example, R² is a C₁-C₄haloalkyl. In some embodiments of any one of the aspects described herein, R² is —CF₃, —CF₂CF₃, —CF₂CF₂CF₃ or —CF₂(CF₃)₂.

In some embodiments of any one of the aspects described herein, R² is —OCH(CH₂OR³²⁸)CH₂OR³²⁹, where R³²⁸ and R³²⁹ independently are H, optionally substituted C₁-C₃₀alkyl, optionally substituted C₂-C₃₀alkenyl or optionally substituted C₂-C₃₀alkynyl. For example, R³²⁸ and R³²⁹ independently are optionally substituted C₁-C₃₀alkyl.

In some embodiments of any one of the aspects described herein, R² is —CH₂C(O)NHR³²¹⁰, where R³²¹⁰ is H, optionally substituted C₁-C₃₀alkyl, optionally substituted C₂-C₃₀alkenyl or optionally substituted C₂-C₃₀alkynyl. For example, R³²¹⁰ is H or optionally substituted C₁-C₃₀alkyl. In some embodiments, R³²¹⁰ is optionally substituted C₁-C₆alkyl.

### R³

In some embodiments of any one of the aspects described herein, R³ is —Z-L²-R⁶, hydrogen, halogen, —OR³³², —SR³³³, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, amino (NH₂), alkylamino, dialkylamino, heterocyclyl, arylamino, diarylamino, heteroarylamino, diheteroarylamino, amino acid, —O(CH₂CH₂O)ᵣCH₂CH₂OR³³⁴, cyano, alkyl-thio-alkyl, thioalkoxy, cycloalkyl, aryl, heteroaryl, —NH(CH₂CH₂NH)ₛCH₂CH₂—R³³⁵, NHC(O)R³³⁶, a lipid, a linker covalently attached to a lipid, a ligand, a linker covalently attached to a ligand, a solid support, a linker covalently attached to a solid support, or a reactive phosphorus group.

In some embodiments of any one of the aspects described herein, R³ is —Z-L²-R⁶, hydrogen, halogen, —OR³²², —SR³²³, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, amino (NH₂), alkylamino, dialkylamino, heterocyclyl, arylamino, diarylamino, heteroarylamino, diheteroarylamino, amino acid, —O(CH₂CH₂O)ᵣCH₂CH₂OR³²⁴, cyano, alkyl-thio-alkyl, thioalkoxy, cycloalkyl, aryl, heteroaryl, —NH(CH₂CH₂NH)ₛCH₂CH₂—R³²⁵, NHC(O)R³²⁴.

In some embodiments of any one of the aspects described herein, R³ is —Z-L²-R⁶, hydrogen, hydroxyl, protected hydroxyl, halogen, optionally substituted C₁₋₃₀ alkyl, optionally substituted C₂₋₃₀alkenyl, optionally substituted C₂₋₃₀alkynyl, optionally substituted C₁₋₃₀ alkoxy, alkoxyalkyl (e.g., methoxy, 2-methoxyethoxy, dimethylaminoethoxyethyoxy, N-methylmethoxyamido), alkoxyalkylamine, alkoxyoxycarboxylate, amino, alkylamino, dialkylamino, —O—C₄₋₃₀alkyl-ON(CH₂R⁸)(CH₂R⁹), or —O—C₄₋₃₀alkyl-ON(CH₂R⁸)(CH₂R⁹). For example, R³ is hydrogen, hydroxyl, protected hydroxyl, halogen, optionally substituted C₁₋₃₀ alkoxy, alkoxyalkyl (e.g., methoxy, 2-methoxyethoxy, dimethylaminoethoxyethyoxy, N-methylmethoxyamido), alkoxyalkylamine, alkoxyoxycarboxylate, amino, alkylamino, or dialkylamino.

In some embodiments of any one of the aspect, R³ is —Z-L²-R⁶, hydrogen, hydroxyl, protected hydroxyl, halogen, optionally substituted C₁₋₃₀ alkoxy, or alkoxyalkyl (e.g., methoxy, 2-methoxyethoxy, dimethylaminoethoxyethyoxy, N-methylmethoxyamido).

In some embodiments of any one of the aspects, R³ is —Z-L²-R⁶, hydrogen, hydroxyl, protected hydroxyl, methoxy, 2-methoxyethoxy, —O-dimethylaminoethoxyethyl (—O-DMAEOE) or —O—N-methylacetamido (—O-NMA).

In some embodiments of any one of the aspects, R³ is hydrogen, hydroxyl, protected hydroxyl or methoxy.

In some embodiments of any one of the aspects, R³ is —Z-L²-R⁶. For example, R³ is —Z-L²-R⁶, where Z is O.

In some embodiments of any one of the aspects, R³ is —Z-L²-R⁶. For example, R³ is —Z-L²-R⁶, where L² is optionally substituted C₁-C₂₀alkylene, optionally substituted C₂-C₂₀alkenylene or optionally substituted C₂-C₂₀alkynylene, and where the backbone of the alkylene, alkenylene or alkynylene can be interrupted or terminated by O, S, S(O), SO₂, NR¹, NR¹—C(O), C(O), C(O)O, cleavable linking group, substituted or unsubstituted aryl, substituted or unsubstituted heteroaryl, substituted or unsubstituted heterocyclic; where R^{N1} is hydrogen, acyl, aliphatic or substituted aliphatic.

In some embodiments of any one of the aspects, R³ is —Z-L²-R⁶, where L² is a polyethylene glycol (PEG).

In some embodiments of any one of the aspects, R³ is —Z-L²-R⁶, where Z is O and L² is optionally substituted C₁-C₂₀alkylene, optionally substituted C₂-C₂₀alkenylene or optionally substituted C₂-C₂₀alkynylene, and where the backbone of the alkylene, alkenylene or alkynylene can be interrupted or terminated by O, S, S(O), SO₂, NR¹, NR¹—C(O), C(O), C(O)O, cleavable linking group, substituted or unsubstituted aryl, substituted or unsubstituted heteroaryl, substituted or unsubstituted heterocyclic; where R^{N1} is hydrogen, acyl, aliphatic or substituted aliphatic.

In some embodiments of any one of the aspects, R³ is —Z-L²-R⁶, where Z is O and L² is a polyethylene glycol (PEG).

In some embodiments of any one of the aspects, R³ is —Z-L²-R⁶, where R⁶ is —O—N(R⁷)R^{7′} or —O—N═C(R⁷)R^{7′}. For example, R³ is —O-L²-O—N(R⁷)R^{7′} or —O-L²-O—N═C(R⁷)R^{7′}, where L² is optionally substituted C₁-C₂₀alkylene, optionally substituted C₂-C₂₀alkenylene or optionally substituted C₂-C₂₀alkynylene, and where the backbone of the alkylene, alkenylene or alkynylene can be interrupted or terminated by O, S, S(O), SO₂, NR¹, NR¹—C(O), C(O), C(O)O, cleavable linking group, substituted or unsubstituted aryl, substituted or unsubstituted heteroaryl, substituted or unsubstituted heterocyclic; where R^{N1} is hydrogen, acyl, aliphatic or substituted aliphatic.

In some embodiments of any one of the aspects, R³ is —Z-L²-R⁶, where R⁶ is —O—N(R⁷)R^{7′}. For example, R³ is —Z-L²-R⁶, where R⁶ is —O—N(R⁷)R^{7′}, where R⁷ and R^{7′} are independently H or a ligand, (e.g., a ligand selected independently from the group consisting of carbohydrates, lipids, vitamins, peptides, proteins, lipoproteins, peptidomimetics, polyamines, nucleosides and nucleotides, oligonucleotides, therapeutic agents, diagnostic agents, detectable labels, antibodies or fragments thereof, optionally substituted C₁₋₃₀ alkyl, optionally substituted C₁₋₃₀ alkenyl, optionally substituted C₁₋₃₀alkynyl, optionally substituted cycloalkyl, optionally substituted heterocyclyl, optionally substituted aryl, optionally substituted heteroaryl, polyethylene glycols (PEGs), nitrogen protecting group.

In some embodiments of any one of the aspects, R³ is —Z-L²-R⁶, where R⁶ is —O—N(R⁷)R^{7′}, where R⁷ and R^{7′} together with the N they are attached to form an optionally substituted heterocyclyl (e.g., phthalimide or morpholine).

In some embodiments of any one of the aspects, R³ is —Z-L²-R⁶, where R⁶ is —O—N═C(R⁷)R⁷.

In some embodiments of any one of the aspects, R³ is a reactive phosphorous group. Optionally, only one of R² and R³ is a reactive phosphorous group.

In some embodiments of any one of the aspects descried herein, R³ is a solid support or a linker covalently attached to a solid support. For example, R³ is —OC(O)CH₂CH₂C(O)NH—Z, where Z is a solid support. Optionally, only one of R² and R³ is a solid support or a linker covalently attached to a solid support.

In some embodiments of any one of the aspects, when R³ is —OR³³², R³³² can be hydrogen or a hydroxyl protecting group. For example, R³³² can be hydrogen in some embodiments of any one of the aspects described herein. In some embodiments, R³ is —OC(O)CH₂CH₂CO₂H.

When R³ is —SR³³³, R³³³ can be hydrogen or a sulfur protecting group. Accordingly, in some embodiments of any one of the aspects, R³³³ is hydrogen.

When R³ is —O(CH₂CH₂O)ᵣCH₂CH₂OR³³⁴, r can be 1-50; R³³⁴ is independently for each occurrence H, C₁-C₃₀alkyl, cyclyl, heterocyclyl, aryl, heteroaryl, aralkyl, sugar or R³³⁵; and R³³⁵ is independently for each occurrence amino (NH₂), alkylamino, dialkylamino, arylamino, diarylamino, heteroarylamino, or diheteroaryl amino.

When R³ is —NH(CH₂CH₂NH)ₛCH₂CH₂—R³³⁵, s can be 1-50 and R³³⁵ can be independently for each occurrence amino (NH₂), alkylamino, dialkylamino, arylamino, diarylamino, heteroarylamino, or diheteroaryl amino.

In some embodiments of any one of the aspects described herein, R³ is hydrogen, halogen, —OR³³², or optionally substituted C₁-C₃₀alkoxy. For example, R³ is halogen, —OR³³², or optionally substituted C₁-C₃₀alkoxy. In some embodiments of any one of the aspects described herein, R³ is F, OH or optionally substituted C₁-C₃₀alkoxy.

In some embodiments of any one of the aspects described herein, R³ is C₁-C₃₀alkoxy optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]2, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)— alkyl, C(O)— alkyl, alkylcarbonylaminyl, CH₂—[CH(OH)]m-(CH₂)ₚ—OH, CH₂—[CH(OH)]m-(CH₂)ₚ—NH₂ or CH₂-aryl-alkoxy, where “m” and “p” are independently 1, 2, 3, 4, 5 or 6. For example, R³ is C₁-C₃₀alkoxy optionally substituted with a NH₂, OH, C(O)NH₂, COOH, halo, SH, or C₁-C₆alkoxy. In some embodiments of any one of the aspects described herein, R³ is —O(CH₂)ₜCH₃, where t is 1-21.

In some embodiments of any one of the aspects, R³ is —O(CH₂)ᵤR³³⁷, where u is 2-10; R³³⁷ is C₁-C₆alkoxy, amino (NH₂), CO₂H, OH or halo. For example, R³³⁷ is —CH₃ or NH₂. Accordingly, in some embodiments of any one of the aspects described herein, R³ is —O(CH₂)ᵤ—OMe or R³ is —O(CH₂)ᵤNH₂

In some embodiments of any one of the aspects described herein, R³ is a C₁-C₆haloalkyl. For example, R³ is a C₁-C₄haloalkyl. In some embodiments of any one of the aspects described herein, R³ is —CF₃, —CF₂CF₃, —CF₂CF₂CF₃ or —CF₂(CF₃)₂.

In some embodiments of any one of the aspects described herein, R³ is —OCH(CH₂OR³³⁸)CH₂OR³³⁹, where R³³⁸ and R³³⁹ independently are H, optionally substituted C₁-C₃₀alkyl, optionally substituted C₂-C₃₀alkenyl or optionally substituted C₂-C₃₀alkynyl. For example, R³³⁸ and R³³⁹ independently are optionally substituted C₁-C₃₀alkyl.

In some embodiments of any one of the aspects described herein, R³ is —CH₂C(O)NHR³³¹⁰, where R³³¹⁰ is H, optionally substituted C₁-C₃₀alkyl, optionally substituted C₂-C₃₀alkenyl or optionally substituted C₂-C₃₀alkynyl.

In some embodiments of any one of the aspected described herein, R³ and R⁴ taken together with the atoms to which they are attached form an optionally substituted C₃₋₈cycloalkyl, optionally substituted C₃₋₈cycloalkenyl, or optionally substituted 3-8 membered heterocyclyl.

### R⁴

In some embodiments of any one of the aspects described herein, R⁴ can be —Z-L²-R⁶, hydrogen, optionally substituted C₁₋₆alkyl, optionally substituted C₂₋₆alkenyl, optionally substituted C₂₋₆alkynyl, or optionally substituted C₁₋₆alkoxy. For example, R⁴ can be hydrogen, optionally substituted C₁₋₆alkyl or optionally substituted C₁₋₆alkoxy.

In some embodiments of any one of the aspects, R⁴ is —Z-L²-R⁶. For example, R⁴ is —Z-L²-R⁶, where Z is a bond. In other words, R⁴ is -L²-R⁶.

In some embodiments of any one of the aspects, R⁴ is -L²-R⁶. For example, R⁴ is -L²-R⁶, where L² is optionally substituted C₁-C₂₀alkylene, optionally substituted C₂-C₂₀alkenylene or optionally substituted C₂-C₂₀alkynylene, and where the backbone of the alkylene, alkenylene or alkynylene can be interrupted or terminated by O, S, S(O), SO₂, NR¹, NR¹—C(O), C(O), C(O)O, cleavable linking group, substituted or unsubstituted aryl, substituted or unsubstituted heteroaryl, substituted or unsubstituted heterocyclic; where R^{N1} is hydrogen, acyl, aliphatic or substituted aliphatic.

In some embodiments of any one of the aspects, R⁴ is -L²-R⁶, where R⁶ is —O—N(R⁷)R^{7′} or —O—N═C(R⁷)R^{7′}. For example, R⁴ is -L²-O—N(R⁷)R^{7′} or -L²-O—N═C(R⁷)R^{7′}, where L² is optionally substituted C₁-C₂₀alkylene, optionally substituted C₂-C₂₀alkenylene or optionally substituted C₂-C₂₀alkynylene, and where the backbone of the alkylene, alkenylene or alkynylene can be interrupted or terminated by O, S, S(O), SO₂, NR¹, NR¹—C(O), C(O), C(O)O, cleavable linking group, substituted or unsubstituted aryl, substituted or unsubstituted heteroaryl, substituted or unsubstituted heterocyclic; where R^{N1} is hydrogen, acyl, aliphatic or substituted aliphatic.

In some embodiments of any one of the aspects, R⁴ is -L²-R⁶, where R⁶ is —O—N(R⁷)R^{7′}. For example, R³ is -L²-R⁶, where R⁶ is —O—N(R⁷)R^{7′}, where R⁷ and R^{7′} are independently H or a ligand, (e.g., a ligand selected independently from the group consisting of carbohydrates, lipids, vitamins, peptides, proteins, lipoproteins, peptidomimetics, polyamines, nucleosides and nucleotides, oligonucleotides, therapeutic agents, diagnostic agents, detectable labels, antibodies or fragments thereof, optionally substituted C₁₋₃₀ alkyl, optionally substituted C₁₋₃₀ alkenyl, optionally substituted C₁₋₃₀alkynyl, optionally substituted cycloalkyl, optionally substituted heterocyclyl, optionally substituted aryl, optionally substituted heteroaryl, polyethylene glycols (PEGs), nitrogen protecting group.

In some embodiments of any one of the aspects, R³ is -L²-R⁶, where R⁶ is —O—N(R⁷)R^{7′}, where R⁷ and R^{7′} together with the N they are attached to form an optionally substituted heterocyclyl (e.g., phthalimide or morpholine).

In some embodiments of any one of the aspects, R³ is -L²-R⁶, where R⁶ is —O—N═C(R⁷)R⁷.

In some embodiments of any one of the aspects described herein, R⁴ is H.

### R⁵

In some embodiments of the various aspects described herein, R⁵ is R⁶, —Z-L²-R⁶, R⁵⁵¹, hydrogen, hydroxyl, optionally substituted C₁₋₆alkyl-R⁵⁵¹, optionally substituted —C₂₋₆alkenyl-R⁵⁵¹, or optionally substituted —C₂₋₆alkynyl-R⁵⁵¹, where R⁵⁵¹ can be —OR⁵⁵², —SR⁵⁵³, hydrogen, a phosphorous group, a solid support or a linker to a solid support. When R⁵⁵¹ is —OR⁵⁵², R⁵⁵² can be H or a hydroxyl protecting group. Similarly, when R⁵⁵¹ is —SR⁵⁵³, R⁵⁵³ can be H or a sulfur protecting group.

In some embodiments of any one of the aspects, R⁵ is R⁶. For example, R⁵ is —O—N(R⁷)R^{7′} or —O—N═C(R⁷)R^{7′}.

In some embodiments of any one of the aspects, R⁵ is —O—N(R⁷)R^{7′}. For example, R⁵ is —O—N(R⁷)R^{7′}, where R⁷ and R^{7′} are independently H or a ligand, (e.g., a ligand selected independently from the group consisting of carbohydrates, lipids, vitamins, peptides, proteins, lipoproteins, peptidomimetics, polyamines, nucleosides and nucleotides, oligonucleotides, therapeutic agents, diagnostic agents, detectable labels, antibodies or fragments thereof, optionally substituted C₁₋₃₀ alkyl, optionally substituted C₁₋₃₀ alkenyl, optionally substituted C₁₋₃₀alkynyl, optionally substituted cycloalkyl, optionally substituted heterocyclyl, optionally substituted aryl, optionally substituted heteroaryl, polyethylene glycols (PEGs), nitrogen protecting group.

In some embodiments of any one of the aspects, R⁵ is —O—N(R⁷)R^{7′}, where R⁷ and R^{7′} together with the N they are attached to form an optionally substituted heterocyclyl (e.g., phthalimide or morpholine).

In some embodiments of any one of the aspects, R⁵ is —O—N═C(R⁷)R^{7′}.

In some embodiments of any one of the aspects, R⁵ is —Z-L²-R⁶. For example, R⁵ is —Z-L²-R⁶, where Z is O.

In some embodiments of any one of the aspects, R⁵ is —Z-L²-R⁶. For example, R⁵ is —Z-L²-R⁶, where L² is optionally substituted C₁-C₂₀alkylene, optionally substituted C₂-C₂₀alkenylene or optionally substituted C₂-C₂₀alkynylene, and where the backbone of the alkylene, alkenylene or alkynylene can be interrupted or terminated by O, S, S(O), SO₂, NR¹, NR¹—C(O), C(O), C(O)O, cleavable linking group, substituted or unsubstituted aryl, substituted or unsubstituted heteroaryl, substituted or unsubstituted heterocyclic; where R^{N1} is hydrogen, acyl, aliphatic or substituted aliphatic.

In some embodiments of any one of the aspects, R⁵ is —Z-L²-R⁶, where L² is a polyethylene glycol (PEG).

In some embodiments of any one of the aspects, R⁵ is —Z-L²-R⁶, where Z is O and L² is optionally substituted C₁-C₂₀alkylene, optionally substituted C₂-C₂₀alkenylene or optionally substituted C₂-C₂₀alkynylene, and where the backbone of the alkylene, alkenylene or alkynylene can be interrupted or terminated by O, S, S(O), SO₂, NR¹, NR¹—C(O), C(O), C(O)O, cleavable linking group, substituted or unsubstituted aryl, substituted or unsubstituted heteroaryl, substituted or unsubstituted heterocyclic; where R^{N1} is hydrogen, acyl, aliphatic or substituted aliphatic.

In some embodiments of any one of the aspects, R⁵ is —Z-L²-R⁶, where Z is O and L² is a polyethylene glycol (PEG).

In some embodiments of any one of the aspects, R⁵ is —Z-L²-R⁶, where R⁶ is —O—N(R⁷)R^{7′} or —O—N═C(R⁷)R^{7′}. For example, R⁵ is —O-L²-O—N(R⁷)R^{7′} or —O-L²-O—N═C(R⁷)R⁷, where L² is optionally substituted C₁-C₂₀alkylene, optionally substituted C₂-C₂₀alkenylene or optionally substituted C₂-C₂₀alkynylene, and where the backbone of the alkylene, alkenylene or alkynylene can be interrupted or terminated by O, S, S(O), SO₂, NR¹, NR¹—C(O), C(O), C(O)O, cleavable linking group, substituted or unsubstituted aryl, substituted or unsubstituted heteroaryl, substituted or unsubstituted heterocyclic; where R^{N1} is hydrogen, acyl, aliphatic or substituted aliphatic.

In some embodiments of any one of the aspects, R⁵ is —Z-L²-R⁶, where R⁶ is —O—N(R⁷)R^{7′}. For example, R⁵ is —Z-L²-R⁶, where R⁶ is —O—N(R⁷)R^{7′}, where R¹ and R^{7′} are independently H or a ligand, (e.g., a ligand selected independently from the group consisting of carbohydrates, lipids, vitamins, peptides, proteins, lipoproteins, peptidomimetics, polyamines, nucleosides and nucleotides, oligonucleotides, therapeutic agents, diagnostic agents, detectable labels, antibodies or fragments thereof, optionally substituted C₁₋₃₀ alkyl, optionally substituted C₁₋₃₀ alkenyl, optionally substituted C₁₋₃₀alkynyl, optionally substituted cycloalkyl, optionally substituted heterocyclyl, optionally substituted aryl, optionally substituted heteroaryl, polyethylene glycols (PEGs), nitrogen protecting group.

In some embodiments of any one of the aspects, R⁵ is —Z-L²-R⁶, where R⁶ is —O—N(R⁷)R^{7′}, where R⁷ and R^{7′} together with the N they are attached to form an optionally substituted heterocyclyl (e.g., phthalimide or morpholine).

In some embodiments of any one of the aspects, R⁵ is —Z-L²-R⁶, where R⁶ is —O—N═C(R⁷)R⁷.

In some embodiments of any one of the aspects described herein, R⁵ is —OR⁵⁵² or —SR⁵⁵³.

In some embodiments of any one of the aspects described herein, R⁵⁵² is a hydroxyl protecting group. Exemplary hydroxyl protecting groups for R⁵⁵² include, but are not limited to, benzyl, benzoyl, 2,6-dichlorobenzyl, t-butyldimethylsilyl, t-butyldiphenylsilyl, mesylate, tosylate, 4,4′-dimethoxytrityl (DMT), 9-phenylxanthine-9-yl (Pixyl) and 9-(p-methoxyphenyl)xanthine-9-yl (MOX). In some embodiments of any one of the aspects described herein, R⁵ is —OR⁵⁵² and R⁵⁵² is 4,4′-dimethoxytrityl (DMT), e.g., R⁵ is —O-DMT.

In some embodiments of any one of the aspects described herein, R⁵ is —CH(R⁵⁵⁴)—R⁵⁵¹, where R⁵⁵⁴ is hydrogen, halogen, optionally substituted C₁-C₃₀alkyl, optionally substituted C₂-C₃₀alkenyl, optionally substituted C₂-C₃₀alkynyl, or optionally substituted C₁-C₃₀alkoxy.

In some embodiments of any one of the aspects, when R⁵ is —CH(R⁵⁵⁴)—R⁵⁵¹, R⁵⁵⁴ is H or C₁-C₃₀alkyl optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]2, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)— alkyl, C(O)— alkyl, alkylcarbonylaminyl, CH₂—[CH(OH)]m-(CH₂)ₚ—OH, CH₂—[CH(OH)]m-(CH₂)ₚ—NH₂ or CH₂-aryl-alkoxy, where “m” and “p” are independently 1, 2, 3, 4, 5 or 6. For example, R⁵⁵⁴ is H. In some other non-limiting examples, R⁵⁵⁴ is C₁-C₃₀alkyl optionally substituted with a NH₂, OH, C(O)NH₂, COOH, halo, SH, or C₁-C₆alkoxy.

In some embodiments of the various aspects described herein, R⁵ is —CH(R⁵⁵⁴)—O—R⁵⁵² where R⁵⁵⁴ is H or C₁-C₃₀alkyl optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]2, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)— alkyl, C(O)— alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy, where “m” and “p” are independently 1, 2, 3, 4, 5 or 6. For example, R⁵⁵⁴ is H. In some other non-limiting examples, R¹⁵⁴ is C₁-C₃₀alkyl optionally substituted with a NH₂, OH, C(O)NH₂, COOH, halo, SH, or C₁-C₆alkoxy.

In some embodiments of the various aspects described herein, R⁵ is optionally substituted C₁₋₆alkyl-R⁵⁵¹ or optionally substituted —C₂₋₆alkenyl-R⁵⁵¹,

In some embodiments of any one of the aspects described herein, R⁵ is —C(R⁵⁵⁴)═CHR⁵⁵¹. It is noted that the double bond in —C(R⁵⁵⁴)═CHR⁵⁵¹ can be in the cis or trans configuration. Accordingly, in some embodiments of any one of the aspects, R^{d} is —C(R⁵⁵⁴)=CHR⁵⁵¹ and wherein the double bond is in the cis configuration. In some other embodiments of any one of the aspects, R^{d} is —C(R⁵⁵⁴)=CHR⁵⁵¹ and wherein the double bond is in the trans configuration.

In some embodiments of any one of the aspects described herein, R⁵ is —CH═CHR⁵⁵¹.

In some embodiments of any one of the aspects, when R⁵ is —C(R⁵⁵⁴)=CHR⁵⁵¹, R⁵⁵⁴ is H or C₁-C₃₀alkyl optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]2, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)— alkyl, C(O)— alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy, where “m” and “p” are independently 1, 2, 3, 4, 5 or 6; and R⁵⁵¹ is a phosphorous group. For example, R⁵ is —CH═CHR⁵⁵¹.

In some embodiments of any one of the aspects described herein, R⁵⁵¹ is a reactive phosphorous group.

In some embodiments of any one of the aspects, R⁵ is —CH═CH—P(O)(OR⁵⁵⁵)₂, —CH═CH—P(S)(OR⁵⁵⁵)₂, —CH═CH—P(S)(SR⁵⁵⁶)(OR⁵⁵⁵), —CH═CH—P(S)(SR⁵⁵⁶)₂, —CH═CH—OP(O)(OR⁵⁵⁵)₂, —CH═CH—OP(S)(OR⁵⁵⁵)₂, —CH═CH—OP(S)(SR⁵⁵⁶)(OR⁵⁵⁵), —CH═CH—OP(S)(SR⁵⁵⁶)₂, —CH═CH—SP(O)(OR⁵⁵⁵)₂, —CH═CH—SP(S)(OR⁵⁵⁵)₂, —CH═CH—SP(S)(SR⁵⁵⁶)(OR⁵⁵), or —CH═CH—SP(S)(SR⁵⁵⁶)₂, where each R⁵⁵⁵ is independently hydrogen, optionally substituted C₁₋₃₀alkyl, optionally substituted C₂₋₃₀alkenyl, or optionally substituted C₂₋₃₀ alkynyl, or an oxygen-protecting group; and each R⁵⁵⁶ is independently hydrogen, optionally substituted C₁₋₃₀alkyl, optionally substituted C₂₋₃₀alkenyl, or optionally substituted C₂₋₃₀alkynyl, or a sulfur-protecting group.

In some embodiments of any one of the aspects, at least one R⁵⁵⁵ in —P(O)(OR⁵⁵⁵)₂, —P(S)(OR⁵⁵⁵)₂, —P(S)(SR⁵⁵⁶)(OR⁵⁵⁵), —OP(O)(OR⁵⁵⁵)₂, —OP(S)(OR⁵⁵⁵)₂, —OP(S)(SR⁵⁵⁶)(OR⁵⁵⁵), SP(O)(OR⁵⁵⁵)₂, —SP(S)(OR⁵⁵⁵)₂, and —SP(S)(SR⁵⁵⁶)(OR⁵⁵⁵) is hydrogen.

In some other embodiments of any one of the aspects, at least one R⁵⁵⁵ in —P(O)(OR⁵⁵⁵)₂, —P(S)(OR⁵⁵⁵)₂, —P(S)(SR⁵⁵⁶)(OR⁵⁵⁵), —OP(O)(OR⁵⁵⁵)₂, —OP(S)(OR⁵⁵⁵)₂, —OP(S)(SR⁵⁵⁶)(OR⁵⁵⁵), SP(O)(OR⁵⁵⁵)₂, —SP(S)(OR⁵⁵⁵)₂, or —SP(S)(SR⁵⁵⁶)(OR⁵⁵⁵) is not hydrogen. For example, at least one at least one R⁵⁵⁵ in P(O)(OR⁵⁵⁵)₂, —P(S)(OR⁵⁵⁵)₂, —P(S)(SR⁵⁵⁶)(OR⁵⁵⁵), —OP(O)(OR⁵⁵⁵)₂, —OP(S)(OR⁵⁵⁵)₂, —OP(S)(SR⁵⁵⁶)(OR⁵⁵⁵), SP(O)(OR⁵⁵⁵)₂, —SP(S)(OR⁵⁵⁵)₂, and —SP(S)(SR⁵⁵⁶)(OR⁵⁵⁵) is optionally substituted C₁₋₃₀alkyl, optionally substituted C₂₋₃₀alkenyl, or optionally substituted C₂₋₃₀alkynyl, or an oxygen-protecting group.

In some embodiments of any one of the aspects, at least one R⁵⁵⁵ is H and at least one R⁵⁵⁵ is other than H in —P(O)(OR⁵⁵⁵)₂, —P(S)(OR⁵⁵⁵)₂, —P(S)(SR⁵⁵⁶)(OR⁵⁵⁵), —OP(O)(OR⁵⁵⁵)₂, —OP(S)(OR⁵⁵⁵)₂, —OP(S)(SR⁵⁵⁶)(OR⁵⁵⁵), SP(O)(OR⁵⁵⁵)₂, —SP(S)(OR⁵⁵⁵)₂, and —SP(S)(SR⁵⁵⁶)(OR⁵⁵⁵).

In some embodiments of any one of the aspects, all R⁵⁵⁵ are H in —P(O)(OR⁵⁵⁵)₂, —P(S)(OR⁵⁵⁵)₂, —P(S)(SR⁵⁵⁶)(OR⁵⁵⁵), —OP(O)(OR⁵⁵⁵)₂, —OP(S)(OR⁵⁵⁵)₂, —OP(S)(SR⁵⁵⁶)(OR⁵⁵⁵), —OP(S)(SR⁵⁵⁶)₂, —SP(O)(OR⁵⁵⁵)₂, —SP(S)(OR⁵⁵⁵)₂, —SP(S)(SR⁵⁵⁶)(OR⁵⁵⁵), and —SP(S)(SR⁵⁵⁶)₂.

In some embodiments of any one of the aspects, all R⁵⁵⁵ are other than H in in —P(O)(OR⁵⁵⁵)₂, —P(S)(OR⁵⁵⁵)₂, —P(S)(SR⁵⁵⁶)(OR⁵⁵⁵), —OP(O)(OR⁵⁵⁵)₂, —OP(S)(OR⁵⁵⁵)₂, —OP(S)(SR⁵⁵⁶)(OR⁵⁵⁵), —OP(S)(SR⁵⁵⁶)₂, —SP(O)(OR⁵⁵⁵)₂, —SP(S)(OR⁵⁵⁵)₂, —SP(S)(SR⁵⁵⁶)(OR⁵⁵⁵), and —SP(S)(SR⁵⁵⁶)₂.

In some embodiments of any one of the aspects, at least one R⁵⁵⁶ in —P(S)(SR⁵⁵⁶)(OR⁵⁵⁵), —P(S)(SR⁵⁵⁶)₂, —OP(S)(OR⁵⁵⁵)₂, —OP(S)(SR⁵⁵⁶)(OR⁵⁵⁵), —OP(S)(SR⁵⁵⁶)₂, —SP(S)(SR⁵⁵⁶)(OR⁵⁵⁵), and —SP(S)(SR⁵⁵⁶)₂ is H.

In some embodiments of any one of the aspects, at least one R⁵⁵⁶ in —P(S)(SR⁵⁵⁶)(OR⁵⁵⁵), —P(S)(SR⁵⁵⁶)₂, —OP(S)(OR⁵⁵⁵)₂, —OP(S)(SR⁵⁵⁶)(OR⁵⁵⁵), —OP(S)(SR⁵⁵⁶)₂, —SP(S)(SR⁵⁵⁶)(OR⁵⁵⁵), and —SP(S)(SR⁵⁵⁶)₂ is other than H. For example, at least one R⁵⁵⁶ in —P(S)(SR⁵⁵⁶)(OR⁵⁵⁵), —P(S)(SR⁵⁵⁶)₂, —OP(S)(OR⁵⁵⁵)₂, —OP(S)(SR⁵⁵⁶)(OR⁵⁵⁵), —OP(S)(SR⁵⁵⁶)₂, —SP(S)(SR⁵⁵⁶)(OR⁵⁵⁵), and —SP(S)(SR⁵⁵⁶)₂ is optionally substituted C₁₋₃₀alkyl, optionally substituted C₂₋₃₀alkenyl, or optionally substituted C₂₋₃₀alkynyl, or an sulfur-protecting group.

In some embodiments of any one of the aspects, at least one R⁵⁵⁶ is H and at least one R⁵⁵⁶ is other than H in —P(S)(SR⁵⁵⁶)₂, —OP(S)(SR⁵⁵⁶)₂ and —SP(S)(SR⁵⁵⁶)₂.

In some embodiments, all R⁵⁵⁶ are H in —P(S)(SR⁵⁵⁶)(OR⁵⁵⁵), —P(S)(SR⁵⁵⁶)₂, —OP(S)(OR⁵⁵)₂, —OP(S)(SR⁵⁵⁶)(OR⁵⁵⁵), —OP(S)(SR⁵⁵⁶)₂, —SP(S)(SR⁵⁵⁶)(OR⁵⁵), and —SP(S)(SR⁵⁶)₂.

In some embodiments, all R⁵⁵⁶ are other than H in —P(S)(SR⁵⁵⁶)(OR⁵⁵⁵), —P(S)(SR⁵⁵⁶)₂, —OP(S)(OR⁵⁵⁵)₂, —OP(S)(SR⁵⁵⁶)(OR⁵⁵⁵), —OP(S)(SR⁵⁵⁶)₂, —SP(S)(SR⁵⁵⁶)(OR⁵⁵⁵), and —SP(S)(SR⁵⁵⁶)₂.

In some embodiments of any one of the aspects, R³ is a reactive phosphorous group, a solid support, a linker to a solid support, and R⁵ is a protected hydroxyl.

In some other embodiments of any one of the aspects, R² is a reactive phosphorous group, a solid support, a linker to a solid support, and R⁵ is a protected hydroxyl.

In some embodiments of any one of the aspects, R⁵ is R⁶, and one of R² and R³ is a reactive phosphorous group, a solid support, a linker to a solid support.

### R⁶

In some embodiments of any one of the aspects, R⁶ is —O—N(R⁷)R^{7′}.

In some other embodiments of any one of the aspects, R⁶ is —O—N═C(R⁷)R^{7′}.

In some embodiments of any one of the aspects described herein R⁶ is =N—OR^{7′}.

In some other embodiments of any one of the aspects, R⁶ is —O—N═C(R⁷)R^{7′}.

In some embodiments of any one of the aspects described herein R⁶ is —N(R⁷)—OR^{7′} or - or —N(R^{7′})—OR⁷.

R⁷ and R^{7′}

Embodiments of the various aspects described herein include the groups R⁷ and R^{7′}. Each R¹ and R⁷can be independently selected from the groups consisting of H, carbohydrates, lipids, vitamins, peptides, proteins, lipoproteins, peptidomimetics, polyamines, nucleosides and nucleotides, oligonucleotides, detectable labels, diagnostic agents (e.g., biotin), fluorescent dyes, polyethylene glycols (PEGs), antibodies, antibody fragments (e.g., nanobodies).

In some embodiments of any one of the aspects described herein, at least one of R¹ and R¹ is a ligand. Without wishing to be bound by a theory, ligands modify one or more properties of the attached molecule (e.g., the oligonucleotide described herein) including but not limited to pharmacodynamic, pharmacokinetic, binding, absorption, cellular distribution, cellular uptake, charge and clearance. Ligands are routinely used in the chemical arts and are linked directly or via an optional linking moiety or linking group to a parent compound. A preferred list of ligands includes without limitation, intercalators, reporter molecules, polyamines, polyamides, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins and dyes.

Preferred ligands amenable to the present invention include lipid moieties such as a cholesterol moiety (Letsinger et al., Proc. Natl. Acad. Sci. USA, 1989, 86, 6553); cholic acid (Manoharan et al., Bioorg. Med. Chem. Lett., 1994, 4, 1053); a thioether, e.g., hexyl-S-tritylthiol (Manoharan et al., Ann. N.Y. Acad. Sci., 1992, 660, 306; Manoharan et al., Bioorg. Med. Chem. Let., 1993, 3, 2765); a thiocholesterol (Oberhauser et al., Nucl. Acids Res., 1992, 20, 533); an aliphatic chain, e.g., dodecandiol or undecyl residues (Saison-Behmoaras et al., EMBO J., 1991, 10, 111; Kabanov et al., FEBS Lett., 1990, 259, 327; Svinarchuk et al., Biochimie, 1993, 75, 49); a phospholipid, e.g., di-hexadecyl-rac-glycerol or triethylammonium-1,2-di-O-hexadecyl-rac-glycero-3-H-phosphonate (Manoharan et al., Tetrahedron Lett., 1995, 36, 3651; Shea et al., Nucl. Acids Res., 1990, 18, 3777); a polyamine or a polyethylene glycol chain (Manoharan et al., Nucleosides & Nucleotides, 1995, 14, 969); adamantane acetic acid (Manoharan et al., Tetrahedron Lett., 1995, 36, 3651); a palmityl moiety (Mishra et al., Biochim. Biophys. Acta, 1995, 1264, 229); or an octadecylamine or hexylamino-carbonyl-oxycholesterol moiety (Crooke et al., J. Pharmacol. Exp. Ther., 1996, 277, 923).

Ligands can include naturally occurring molecules, or recombinant or synthetic molecules. Exemplary ligands include, but are not limited to, polylysine (PLL), poly L-aspartic acid, poly L-glutamic acid, styrene-maleic acid anhydride copolymer, poly(L-lactide-co-glycolied) copolymer, divinyl ether-maleic anhydride copolymer, N-(2-hydroxylpropyl)methacrylamide copolymer (HMPA), polyethylene glycol (PEG, e.g., PEG-2K, PEG-5K, PEG-10K, PEG-12K, PEG-15K, PEG-20K, PEG-40K), MPEG, [MPEG]₂, polyvinyl alcohol (PVA), polyurethane, poly(2-ethylacryllic acid), N-isopropylacrylamide polymers, polyphosphazine, polyethylenimine, cationic groups, spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, thyrotropin, melanotropin, lectin, glycoprotein, surfactant protein A, mucin, glycosylated polyaminoacids, transferrin, bisphosphonate, polyglutamate, polyaspartate, aptamer, asialofetuin, hyaluronan, procollagen, immunoglobulins (e.g., antibodies), insulin, transferrin, albumin, sugar-albumin conjugates, intercalating agents (e.g., acridines), cross-linkers (e.g. psoralen, mitomycin C), porphyrins (e.g., TPPC4, texaphyrin, Sapphyrin), polycyclic aromatic hydrocarbons (e.g., phenazine, dihydrophenazine), artificial endonucleases (e.g., EDTA), lipophilic molecules (e.g., steroids, bile acids, cholesterol, cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid, O3-(oleoyl)lithocholic acid, O3-(oleoyl)cholenic acid, dimethoxytrityl, or phenoxazine), peptides (e.g., an alpha helical peptide, amphipathic peptide, RGD peptide, cell permeation peptide, endosomolytic/fusogenic peptide), alkylating agents, phosphate, amino, mercapto, polyamino, alkyl, substituted alkyl, radiolabeled markers, enzymes, haptens (e.g. biotin), transport/absorption facilitators (e.g., naproxen, aspirin, vitamin E, folic acid), synthetic ribonucleases (e.g., imidazole, bisimidazole, histamine, imidazole clusters, acridine-imidazole conjugates, Eu3+ complexes of tetraazamacrocycles), dinitrophenyl, HRP, AP, antibodies, hormones and hormone receptors, lectins, carbohydrates, multivalent carbohydrates, vitamins (e.g., vitamin A, vitamin E, vitamin K, vitamin B, e.g., folic acid, B12, riboflavin, biotin and pyridoxal), vitamin cofactors, lipopolysaccharide, an activator of p38 MAP kinase, an activator of NF-κB, taxon, vincristine, vinblastine, cytochalasin, nocodazole, japlakinolide, latrunculin A, phalloidin, swinholide A, indanocine, myoservin, tumor necrosis factor alpha (TNFalpha), interleukin-1 beta, gamma interferon, natural or recombinant low density lipoprotein (LDL), natural or recombinant high-density lipoprotein (HDL), and a cell-permeation agent (e.g., a.helical cell-permeation agent).

Peptide and peptidomimetic ligands include those having naturally occurring or modified peptides, e.g., D or L peptides; α, β, or γ peptides; N-methyl peptides; azapeptides; peptides having one or more amide, i.e., peptide, linkages replaced with one or more urea, thiourea, carbamate, or sulfonyl urea linkages; or cyclic peptides. A peptidomimetic (also referred to herein as an oligopeptidomimetic) is a molecule capable of folding into a defined three-dimensional structure similar to a natural peptide. The peptide or peptidomimetic ligand can be about 5-50 amino acids long, e.g., about 5, 10, 15, 20, 25, 30, 35, 40, 45, or 50 amino acids long.

Exemplary amphipathic peptides include, but are not limited to, cecropins, lycotoxins, paradaxins, buforin, CPF, bombinin-like peptide (BLP), cathelicidins, ceratotoxins, S clava peptides, hagfish intestinal antimicrobial peptides (HFIAPs), magainines, brevinins-2, dermaseptins, melittins, pleurocidin, H₂A peptides, Xenopus peptides, esculentinis-1, and caerins.

As used herein, the term “endosomolytic ligand” refers to molecules having endosomolytic properties. Endosomolytic ligands promote the lysis of and/or transport of the composition of the invention, or its components, from the cellular compartments such as the endosome, lysosome, endoplasmic reticulum (ER), Golgi apparatus, microtubule, peroxisome, or other vesicular bodies within the cell, to the cytoplasm of the cell. Some exemplary endosomolytic ligands include, but are not limited to, imidazoles, poly or oligoimidazoles, linear or branched polyethyleneimines (PEIs), linear and branched polyamines, e.g. spermine, cationic linear and branched polyamines, polycarboxylates, polycations, masked oligo or poly cations or anions, acetals, polyacetals, ketals/polyketals, orthoesters, linear or branched polymers with masked or unmasked cationic or anionic charges, dendrimers with masked or unmasked cationic or anionic charges, polyanionic peptides, polyanionic peptidomimetics, pH-sensitive peptides, natural and synthetic fusogenic lipids, natural and synthetic cationic lipids.

Exemplary endosomolytic/fusogenic peptides include, but are not limited to, AALEALAEALEALAEALEALAEAAAAGGC (GALA); AALAEALAEALAEALAEALAEALAAAAGGC (EALA); ALEALAEALEALAEA; GLFEAIEGFIENGWEGMIWDYG (INF-7); GLFGAIAGFIENGWEGMIDGWYG (Inf HA-2); GLFEAIEGFIENGWEGMIDGWYGCGLFEAIEGFIENGWEGMID GWYGC (diINF-7); GLFEAIEGFIENGWEGMIDGGCGLFEAIEGFIENGWEGMIDGGC (diINF-3); GLFGALAEALAEALAEHLAEALAEALEALAAGGSC (GLF); GLFEAIEGFIENGWEGLAEALAEALEALAAGGSC (GALA-INF3); GLF EAI EGFI ENGW EGnI DG K GLF EAI EGFI ENGW EGnI DG (INF-5, n is norleucine); LFEALLELLESLWELLLEA (JTS-1); GLFKALLKLLKSLWKLLLKA (ppTG1); GLFRALLRLLRSLWRLLLRA (ppTG20); WEAKLAKALAKALAKHLAKALAKALKACEA (KALA); GLFFEAIAEFIEGGWEGLIEGC (HA); GIGAVLKVLTTGLPALISWIKRKRQQ (Melittin); H₅WYG; and CHK₆HC.

Without wishing to be bound by theory, fusogenic lipids fuse with and consequently destabilize a membrane. Fusogenic lipids usually have small head groups and unsaturated acyl chains. Exemplary fusogenic lipids include, but are not limited to, 1,2-dileoyl-sn-3-phosphoethanolamine (DOPE), phosphatidylethanolamine (POPE), palmitoyloleoylphosphatidylcholine (POPC), (6Z,9Z,28Z,31Z)-heptatriaconta-6,9,28,31-tetraen-19-ol (Di-Lin), N-methyl(2,2-di((9Z,12Z)-octadeca-9,12-dienyl)-1,3-dioxolan-4-yl)methanamine (DLin-k-DMA) and N-methyl-2-(2,2-di((9Z,12Z)-octadeca-9,12-dienyl)-1,3-dioxolan-4-yl)ethanamine (also referred to as XTC herein).

Synthetic polymers with endosomolytic activity amenable to the present invention are described in U.S. Pat. App. Pub. Nos. 2009/0048410; 2009/0023890; 2008/0287630; 2008/0287628; 2008/0281044; 2008/0281041; 2008/0269450; 2007/0105804; 20070036865; and 2004/0198687, contents of which are hereby incorporated by reference in their entirety.

Exemplary cell permeation peptides include, but are not limited to, RQIKIWFQNRRMKWKK (penetratin); GRKKRRQRRRPPQC (Tat fragment 48-60); GALFLGWLGAAGSTMGAWSQPKKKRKV (signal sequence based peptide); LLIILRRRIRKQAHAHSK (PVEC); GWTLNSAGYLLKINLKALAALAKKIL (transportan); KLALKLALKALKAALKLA (amphiphilic model peptide); RRRRRRRRR (Arg9); KFFKFFKFFK (Bacterial cell wall permeating peptide); LLGDFFRKSKEKIGKEFKRIVQRIKDFLRNLVPRTES (LL-37); SWLSKTAKKLENSAKKRISEGIAIAIQGGPR (cecropin P1); ACYCRIPACIAGERRYGTCIYQGRLWAFCC (α-defensin); DHYNCVSSGGQCLYSACPIFTKIQGTCYRGKAKCCK (β-defensin); RRRPRPPYLPRPRPPPFFPPRLPPRIPPGFPPRFPPRFPGKR-NH2 (PR-39); ILPWKWPWWPWRR-NH2 (indolicidin); AAVALLPAVLLALLAP (RFGF); AALLPVLLAAP (RFGF analogue); and RKCRIVVIRVCR (bactenecin).

Exemplary cationic groups include, but are not limited to, protonated amino groups, derived from e.g., O-AMINE (AMINE=NH₂; alkylamino, dialkylamino, heterocyclyl, arylamino, diaryl amino, heteroaryl amino, or diheteroaryl amino, ethylene diamine, polyamino); aminoalkoxy, e.g., O(CH₂)ₙAMINE, (e.g., AMINE=NH₂; alkylamino, dialkylamino, heterocyclyl, arylamino, diaryl amino, heteroaryl amino, or diheteroaryl amino, ethylene diamine, polyamino); amino (e.g. NH₂; alkylamino, dialkylamino, heterocyclyl, arylamino, diaryl amino, heteroaryl amino, diheteroaryl amino, or amino acid); and NH(CH₂CH₂NH)ₙCH₂CH₂-AMINE (AMINE=NH₂; alkylamino, dialkylamino, heterocyclyl, arylamino, diaryl amino, heteroaryl amino, or diheteroaryl amino).

As used herein the term “targeting ligand” refers to any molecule that provides an enhanced affinity for a selected target, e.g., a cell, cell type, tissue, organ, region of the body, or a compartment, e.g., a cellular, tissue or organ compartment. Some exemplary targeting ligands include, but are not limited to, antibodies, antigens, folates, receptor ligands, carbohydrates, aptamers, integrin receptor ligands, chemokine receptor ligands, transferrin, biotin, serotonin receptor ligands, PSMA, endothelin, GCPII, somatostatin, LDL and HDL ligands.

Carbohydrate based targeting ligands include, but are not limited to, D-galactose, multivalent galactose, N-acetyl-D-galactosamine (GalNAc), multivalent GalNAc, e.g. GalNAc2 and GalNAc3; D-mannose, multivalent mannose, multivalent lactose, N-acetyl-gulucosamine, multivalent fucose, glycosylated polyaminoacids and lectins. The term multivalent indicates that more than one monosaccharide unit is present. Such monosaccharide subunits can be linked to each other through glycosidic linkages or linked to a scaffold molecule.

A number of folate and folate analogs amenable to the present invention as ligands are described in U.S. Pat. Nos. 2,816,110; 5,552,545; 6,335,434 and 7,128,893, contents of which are herein incorporated in their entireties by reference.

As used herein, the terms “PK modulating ligand” and “PK modulator” refers to molecules which can modulate the pharmacokinetics of oligonucleotides described herein. Some exemplary PK modulator include, but are not limited to, lipophilic molecules, bile acids, sterols, phospholipid analogues, peptides, protein binding agents, vitamins, fatty acids, phenoxazine, aspirin, naproxen, ibuprofen, suprofen, ketoprofen, (S)-(+)-pranoprofen, carprofen, PEGs, biotin, and transthyretia-binding ligands (e.g., tetraiidothyroacetic acid, 2, 4, 6-triiodophenol and flufenamic acid). Oligomeric compounds that comprise a number of phosphorothioate intersugar linkages are also known to bind to serum protein, thus short oligomeric compounds, e.g. oligonucleotides of comprising from about 5 to 30 nucleotides (e.g., 5 to 25 nucleotides, preferably 5 to 20 nucleotides, e.g., 5, 6, 7, 8, 9, 10, 11, 12, 13, 14, 15, 16, 17, 18, 19, or 20 nucleotides), and that comprise a plurality of phosphorothioate linkages in the backbone are also amenable to the present invention as ligands (e.g. as PK modulating ligands). The PK modulating oligonucleotide can comprise at least 3, 4, 5, 6, 7, 8, 9, 10, 11, 12, 13, 14, 15 or more phosphorothioate and/or phosphorodithioate linkages. In some embodiments, all internucleoside linkages in PK modulating oligonucleotide are phosphorothioate and/or phosphorodithioates linkages. In addition, aptamers that bind serum components (e.g. serum proteins) are also amenable to the present invention as PK modulating ligands. Binding to serum components (e.g. serum proteins) can be predicted from albumin binding assays, such as those described in Oravcova, et al., Journal of Chromatography B (1996), 677: 1-27.

When two or more ligands are present, the ligands can all have same properties, all have different properties or some ligands have the same properties while others have different properties. For example, a ligand can have targeting properties, have endosomolytic activity or have PK modulating properties. In a preferred embodiment, all the ligands have different properties.

In some embodiments of any one of the aspects, the ligand has a structure shown in any of Formula (IV)-(VII):

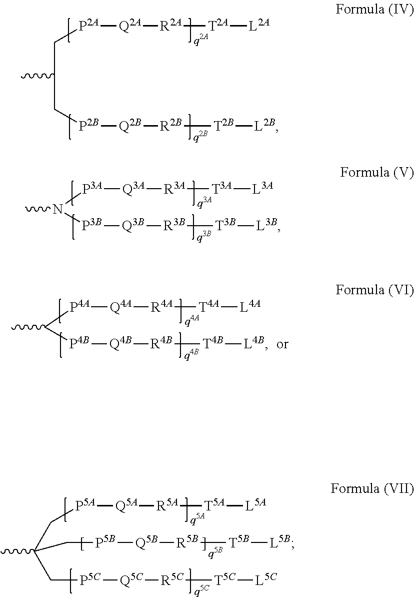

wherein:
q^{2A}, q^{2B}, q^{3A}, q^{3B}, q^{4A}, q^{4B}, q^{5A}, q^{5B} and q^{5C} represent independently for each occurrence 0-20 and wherein the repeating unit can be the same or different; P^{2A}, P^{2B}, P^{3A}, P^{3B}, P^{4A}, P^{4B}, P^{5A}, P^{5B}, P^{5C}, T^{2A}, T^{2B}, T^{3A}, T^{3B}, T^{4A}, T^{4B}, T^{5A}, T^{5B}, T^{5C} are each independently for each occurrence absent, CO, NH, O, S, OC(O), NHC(O), CH₂, CH₂NH or CH₂O;
Q^{2A}, Q^{2B}, Q^{3A}, Q^{3B}, Q^{4A}, Q^{4B}, Q^{5A}, Q^{5B}, Q^{5C} are independently for each occurrence absent, alkylene, substituted alkylene wherein one or more methylenes can be interrupted or terminated by one or more of O, S, S(O), SO₂, N(R^{N}), C(R′)═C(R″), C≡C or C(O);
R^{2A}, R^{2B}, R^{3A}, R^{3B}, R^{4A}, R^{4B}, R^{5A}, R^{5B}, R^{5C} are each independently for each occurrence absent, NH, O, S, CH₂, C(O)O, C(O)NH, NHCH(R^{a})C(O), —C(O)—CH(R^{a})—NH—, CO, CH═N—O,

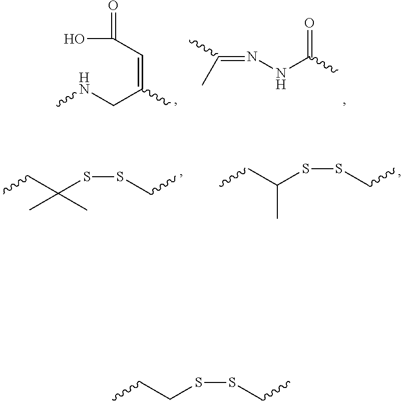

or heterocyclyl;
L^{2A}, L^{2B}, L^{3A}, L^{3B}, L^{4A}, L^{4B}, L^{5A}, L^{5B} and L^{5C} represent the ligand; i.e. each independently for each occurrence a monosaccharide (such as GalNAc), disaccharide, trisaccharide, tetrasaccharide, oligosaccharide, or polysaccharide; and
R^{a} is H or amino acid side chain.

In some embodiments of any one of the aspects, the ligand is of Formula (VII):

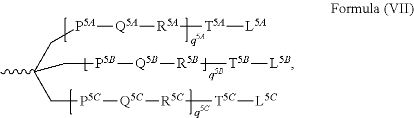

wherein L^{5A} L^{5B} and L^{5C} represent a monosaccharide, such as GalNAc derivative.

Exemplary ligands include, but are not limited to, the following:

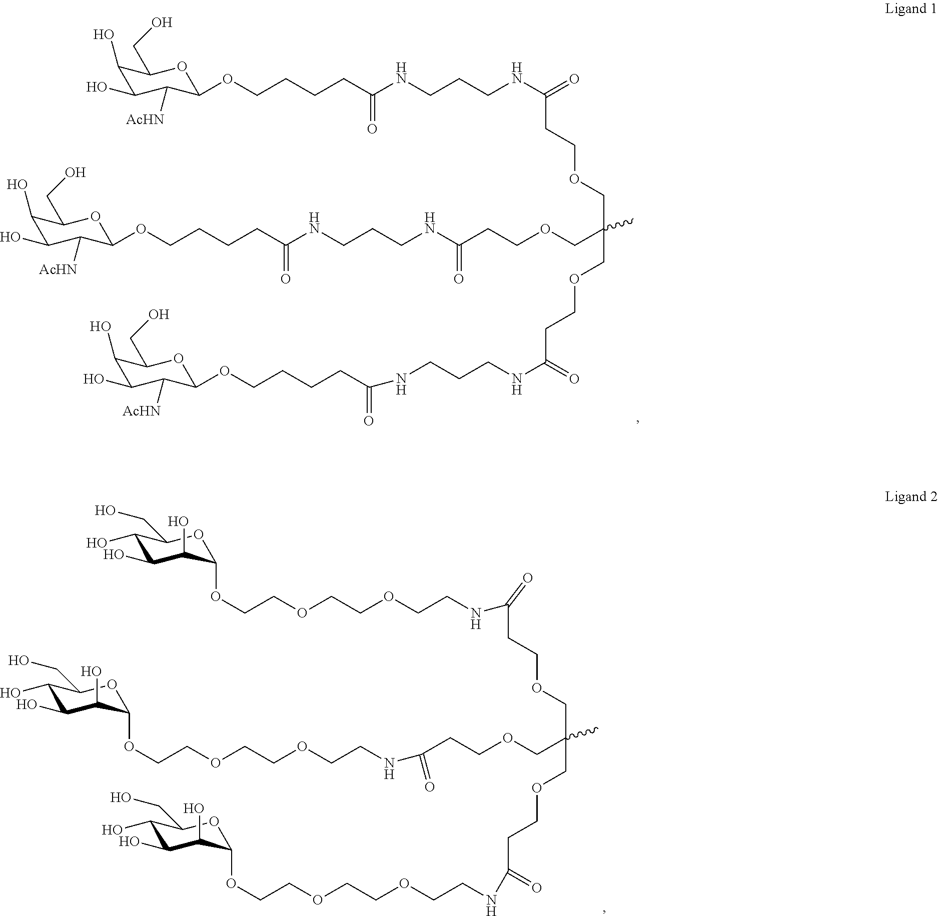

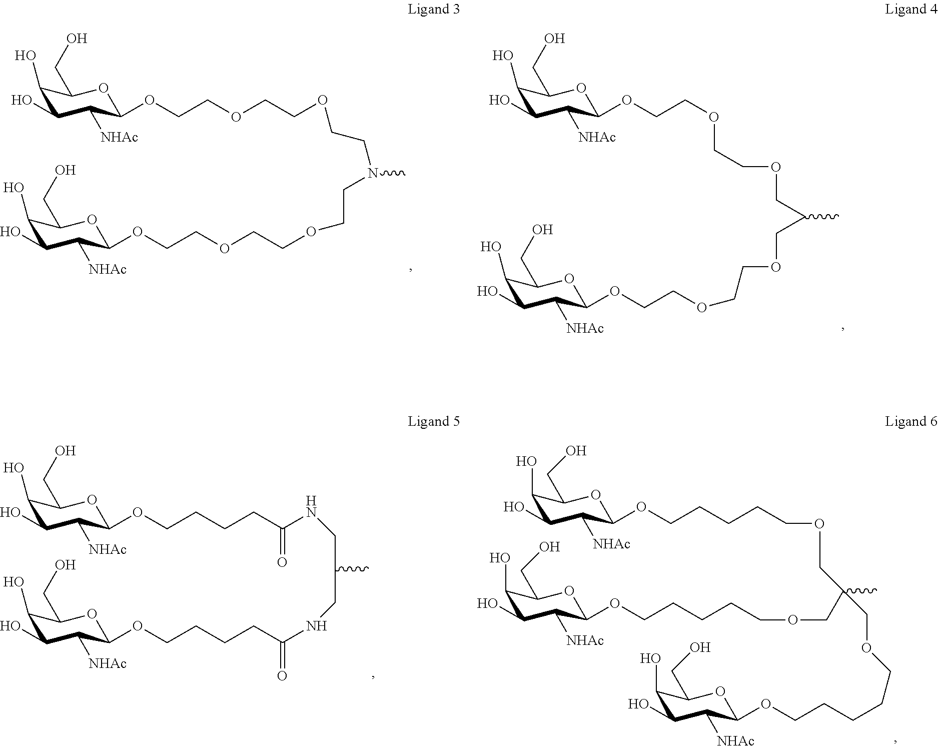

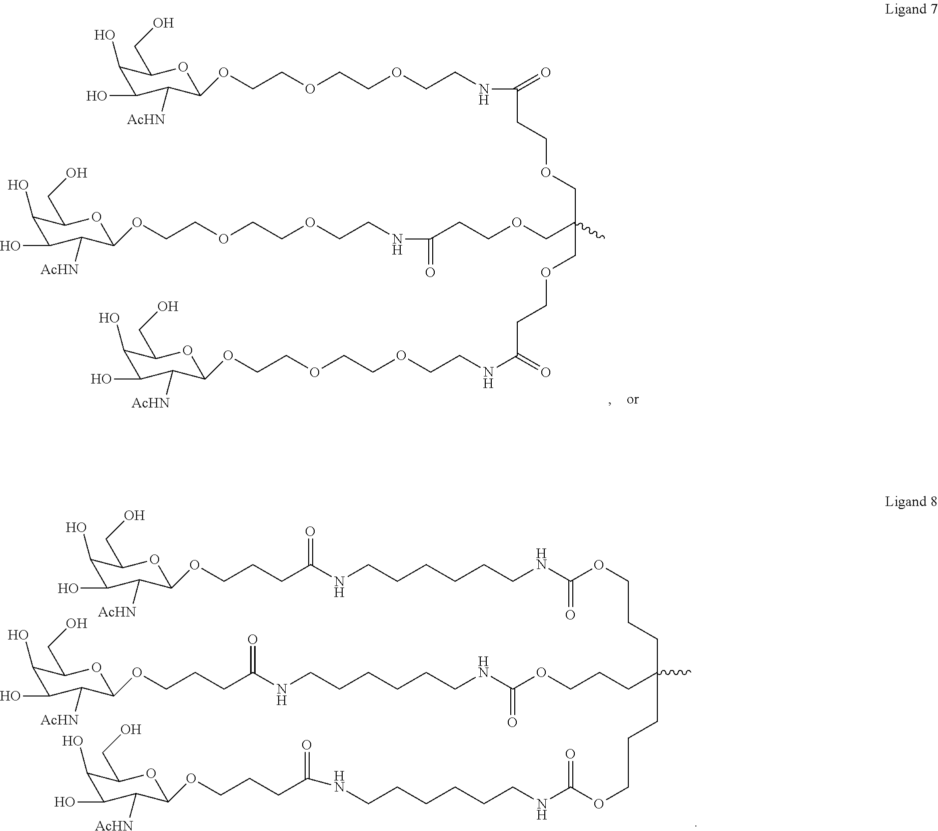

In some embodiments of any one of the aspects described herein, the ligand is a ligand described in U.S. Pat. No. 5,994,517 or U.S. Pat. No. 6,906,182, content of each of which is incorporated herein by reference in its entirety.

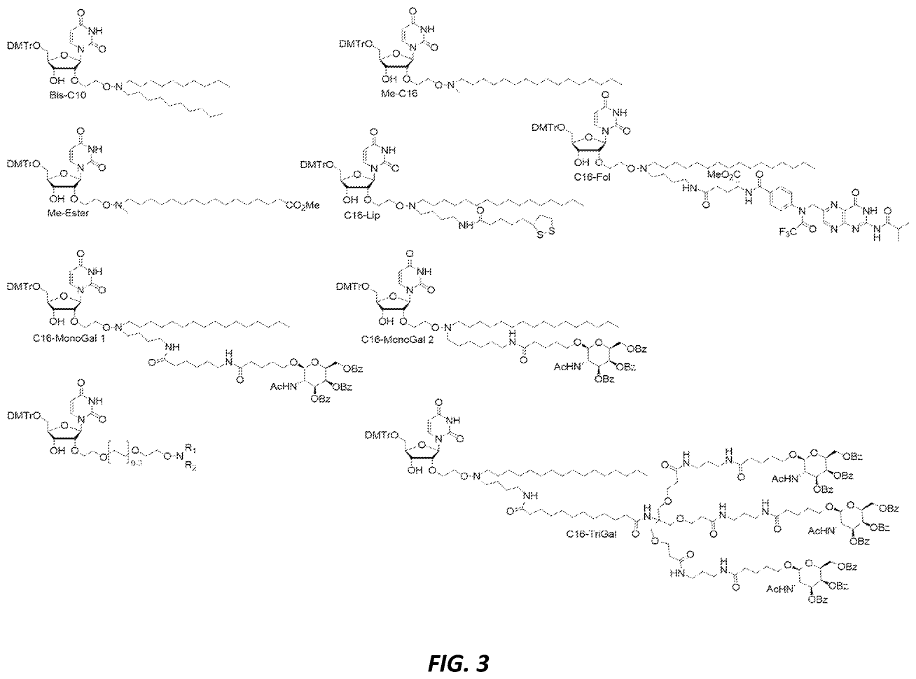

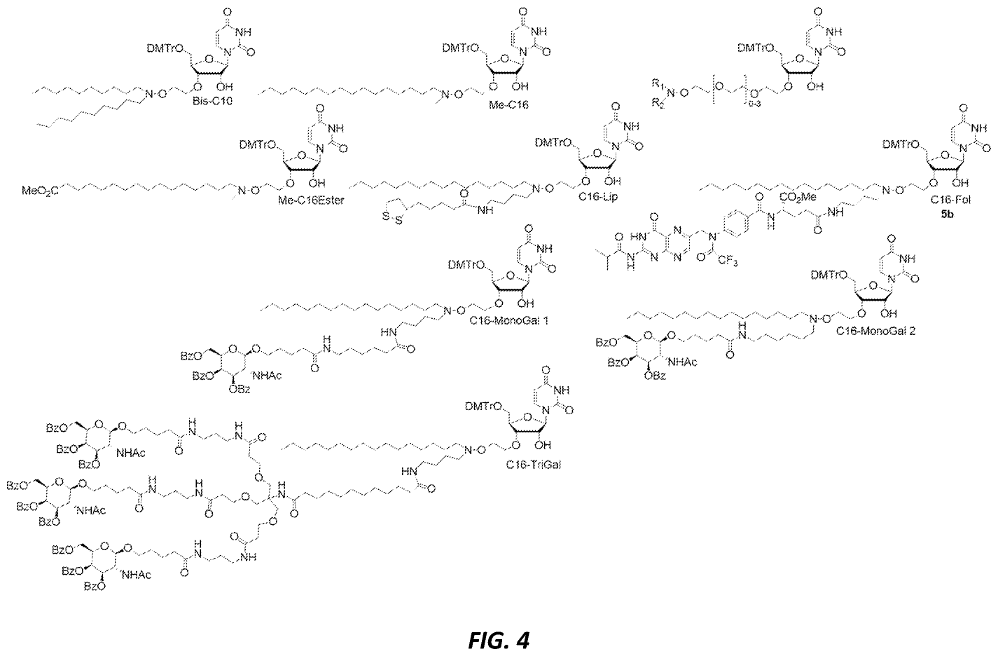

In some embodiments, the ligand can be a tri-antennary ligand described in FIG. 3 of U.S. Pat. No. 6,906,182. For example, the ligand is selected from the following tri-antennary ligands:

Tr-antennery

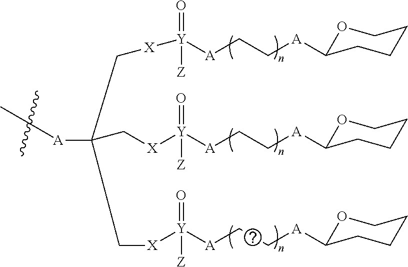

X = NH, O, S
Y = P or 8
Z = NH-alkyl, NH₂, O⁺, S⁺
A = NH, CH₂, O, S
n = 2 to 17 2-carbon units
Carbohydrate =

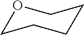

tris((heteroatom)mathyl)-[heteroatom]methane

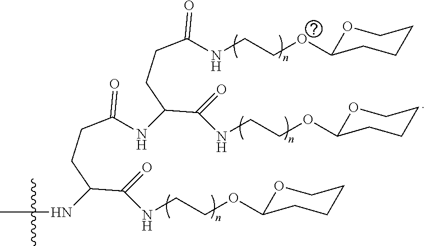

diglutamy

[symbol]

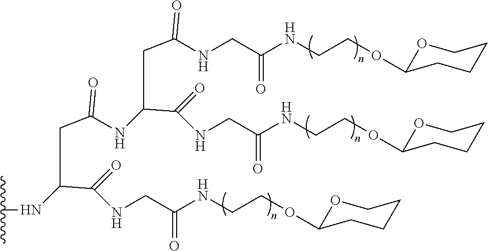

diasparstyl

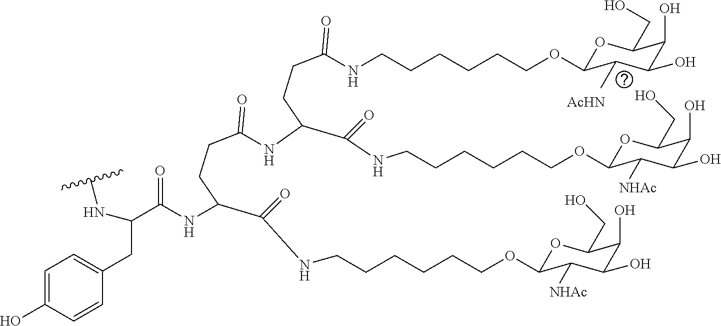 [SMILES: *NC(Cc1ccc(O)cc1)C(=O)NC(CCC(=O)NC(CCC(=O)NCCCCCCOC1CC(O)C(O)C(CO)O1)C(=O)NCCCCCCOC1OC(CO)C(O)C(O)C1NC(C)=O)C(=O)NCCCCCCOC1OC(CO)C(O)C(O)C1NC(C)=O ; "AcHN"]

[symbol]

indicates data missing or illegible when filed

In some embodiments of any one of the aspects described herein, R^{L} is a ligand.

It is noted that when more than one ligands are present, they can be same or different. Accordingly, in some embodiments of any one of the aspects described herein, all the ligands are same. In some other embodiments of any one of the aspects described herein, the ligands are different.

In some embodiments of any one of the aspects, at least one of R⁷ and R^{7′} is selected from the group consisting of carbohydrates, lipids, vitamins, peptides, proteins, lipoproteins, peptidomimetics, polyamines, nucleosides and nucleotides, oligonucleotides, therapeutic agents, diagnostic agents, detectable labels, antibodies or fragments thereof, optionally substituted C₁₋₃₀ alkyl, optionally substituted C₁₋₃₀ alkenyl, optionally substituted C₁₋₃₀alkynyl, optionally substituted cycloalkyl, optionally substituted heterocyclyl, optionally substituted aryl, optionally substituted heteroaryl, polyethylene glycols (PEGs), and nitrogen protecting groups.

In some embodiments of any one of the aspects, at least one of R⁷ and R^{7′} is selected from the group consisting of targeting ligands, endosomolytic ligands and PK modulating ligands.

In some embodiments of any one of the aspects, both of R⁷ and R^{7′} are not H.

It is noted that R⁷ and R^{7′} can be same or different. In some embodiments of any one of the aspects, R⁷ and R^{7′} are same. In some embodiments of any one of the aspects, R⁷ and R^{7′} are different.

In some embodiments of any one of the aspects described herein, each R⁷ and R^{7′} is independently a targeting ligand (e.g., GalNac), a pharmacokinetics modifier, C₁₋₃₀ alkyl, C₁₋₃₀ alkenyl, or C₁₋₃₀alkynyl, each optionally substituted with one or more aldehyde (—C(O)H), carboxylic acid (—COOH), C₁₋₁₀acyl (i.e., —C(O)C₁₋₁₀alkyl), hydroxyl, halogen, cyano, nitro, azido, thiol (i.e., —SH), amino, C₁₋₁₀ alkoxy, C₁₋₁₀ alkylthio, C₁₋₁₀alkylamino, di(C₁₋₁₀alkyl)amino, C₁₋₁₀-alkylcarboxylate (i.e., —C(O)OC₁₋₁₀alkyl), N—(C₁₋₁₀alkyl)amide (i.e., —C(O)NH(C₁₋₁₀alkyl)), N,N-di(C₁₋₁₀alkyl)amide (i.e., —C(O)N(C₁₋₁₀alkyl)₂), amino(C₁₋₁₀)acyl (i.e., —N(H)C(O)(C₁₋₁₀alkyl)), N—(C₁₋₁₀alkyl)amino(C₁₋₁₀)acyl(i.e., —N(C₁₋₁₀alkyl)C(O)(C₁₋₁₀alkyl)), keto (i.e., ═O) or thia (i.e., =S).

In some embodiments of any one of the aspects described herein, at least one (e.g., both of) R⁷ and R^{7′} is selected independently from the group consisting of carbohydrates; peptides; lipids; diagnostic agents (biotin); fluorescent dyes; PEGs; antibody; antibody fragments (Fab, Nanobodies, etc); folic acid and vitamins; RGD-peptides; DUPA ligand, transferrin and transferrin receptor peptides, antibodies and their fragments; edosomolytic small molecules; and endosomolytic peptides.

In some embodiments of any one of the aspects, one of R¹ and R^{7′} is a targeting ligand and the other of R¹ and R^{7′} is a pharmacokinetic modifier.

In some embodiments of any one of the aspects, at least one of R⁷ and R^{7′} is a nitrogen protecting group.

In some embodiments of any one of the aspects, R⁷ and R^{7′} together form a nitrogen protecting group.

In some embodiments of any one of the aspects, R⁷ and R^{7′} together with the N they are attached to form an optionally substituted heterocyclyl (e.g., phthalimide or morpholine).

In some embodiments of any one of the aspects described herein, at least one of R⁷ and R^{7′} is an antibody or an antigen binding fragment thereof.

In some embodiments of any one of the aspects described herein, at least one of R¹ and R^{7′} is an antibody or an antigen binding fragment thereof and the other of R⁷ and R^{7′} is a lipid.

In some embodiments of any one of the aspects described herein, at least one of R⁷ and R^{7′} is an oligonucleotide.

In some embodiments of any one of the aspects described herein, at least one of R⁷ and R^{7′} is an antibody or an antigen binding fragment thereof and the other of R⁷ and R^{7′} is an oligonucleotide.

In some embodiments of any one of the aspects described herein, both of R⁷ and R^{7′} are independently selected oligonucleotides.

In the various aspects described herein, L² can be a linker. For example, L² can be a bond.

In some embodiments of any one of the aspects described herein, L² is an optionally substituted C₁-C₂₀alkylene, (e.g., —(CH₂)_{b}—, where b is 1, 2, 3, 4, 5, 6, 7, 8, 9, 10, 11, 12, 13, 14, 14, 15, 16, 17, 18, 19 or 20), or optionally substituted C₂-C₂₀alkynylene, and where the backbone of the alkylene or alkynylene can be interrupted or terminated by O, S, S(O), SO₂, NR¹, NR¹—C(O), C(O), C(O)O, cleavable linking group, substituted or unsubstituted aryl, substituted or unsubstituted heteroaryl, substituted or unsubstituted heterocyclic; where R^{N1} is hydrogen, acyl, aliphatic or substituted aliphatic. For example, L² is an optionally substituted C₁-C₆alkylene.

In some embodiments of any one of the aspects, L² is an optionally substituted C₁-C₂₀alkylene, where the backbone of the alkylene is interrupted with a heteroaryl (e.g., triazole) or NHC(O).

In some embodiments of any one of the aspects, L² is optionally substituted C₂alkylene, e.g., —CH₂CH₂—.

In some embodiments of any one of the aspects, L² is a bond.

In some embodiments of any one of the aspects, L² is —(CH₂)₉—, —(CH₂)₁₀—, —(CH₂)₁₁—, —(CH₂)₁₂—, or —(CH₂)₁₃—.

In some embodiments of any one of the aspects, L² is —(CH₂)₅—NHC(O)—(CH₂)₁₁—.

In some embodiments of any one of the aspects, L₂ is —CH₂CH₂O—(CH₂)_{LM}—CH₂—, where LM is an integer selected from 1-15. For example, LM s 1, 2, 3, 4, 5, 6, 7, 8, 9, 10, 11, 12, 13, 14 or 15. In some embodiments, LM is an integer selected from 1 to 10. For example, LM is 1, 2, 3, 4, 5, 6, 7, 8 9 or 10.

In some embodiments of any one of the aspects, L₂ is —CH₂C(O)NH—(CH₂)LN-CH₂—, where LN is an integer selected from 1-15. For example, LN s 1, 2, 3, 4, 5, 6, 7, 8, 9, 10, 11, 12, 13, 14 or 15. In some embodiments, LN is an integer selected from 1 to 10. For example, LN is 1, 2, 3, 4, 5, 6, 7, 8 9 or 10.

In some embodiments of any one of the aspects, L² comprises ring formed by an azide-alkyne cycloaddition reaction. For example, L² is an optionally substituted C₁-C₂₀alkylene, where the backbone of the alkylene is interrupted with a ring formed by an azide-alkyne cycloaddition reaction. Thus, in some embodiments of any one of the aspects, L² is —(CH₂)₁₋₁₉-X—(CH₂)₁₋₁₉-, where X is a ring formed by an azide-alkyne cycloaddition reaction. For example, L² is —CH₂—X—(CH₂)₂—, where X is a 1,2,3-triazole.

### R^{3NN}

In some embodiments of any one of the aspects described herein, R^{3NN} is hydrogen, halogen, —OR³³², —SR³³³, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, amino (NH₂), alkylamino, dialkylamino, heterocyclyl, arylamino, diarylamino, heteroarylamino, diheteroarylamino, amino acid, —O(CH₂CH₂O)ᵣCH₂CH₂OR³³⁴, cyano, alkyl-thio-alkyl, thioalkoxy, cycloalkyl, aryl, heteroaryl, —NH(CH₂CH₂NH)ₛCH₂CH₂—R³³⁵, NHC(O)R³³⁶, a lipid, a linker covalently attached to a lipid, a ligand, a linker covalently attached to a ligand, a solid support, a linker covalently attached to a solid support, or a reactive phosphorus group.

In some embodiments of any one of the aspects described herein, R^{3NN} is hydrogen, halogen, —OR³²², —SR³²³, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, amino (NH₂), alkylamino, dialkylamino, heterocyclyl, arylamino, diarylamino, heteroarylamino, diheteroarylamino, amino acid, —O(CH₂CH₂O)ᵣCH₂CH₂OR³²⁴, cyano, alkyl-thio-alkyl, thioalkoxy, cycloalkyl, aryl, heteroaryl, —NH(CH₂CH₂NH)ₛCH₂CH₂—R³²⁵, NHC(O)R³²⁴.

In some embodiments of any one of the aspects described herein, R^{3NN} is hydrogen, hydroxyl, protected hydroxyl, halogen, optionally substituted C₁₋₃₀ alkyl, optionally substituted C₂₋₃₀alkenyl, optionally substituted C₂₋₃₀alkynyl, optionally substituted C₁₋₃₀ alkoxy, alkoxyalkyl (e.g., methoxy, 2-methoxyethoxy, dimethylaminoethoxyethyoxy, N-methylmethoxyamido), alkoxyalkylamine, alkoxyoxycarboxylate, amino, alkylamino, dialkylamino, —O—C₄₋₃₀alkyl-ON(CH₂R⁸)(CH₂R⁹), or —O—C₄₋₃₀alkyl-ON(CH₂R⁸)(CH₂R⁹). For example, R^{3NN} is hydrogen, hydroxyl, protected hydroxyl, halogen, optionally substituted C₁₋₃₀ alkoxy, alkoxyalkyl (e.g., methoxy, 2-methoxyethoxy, dimethylaminoethoxyethyoxy, N-methylmethoxyamido), alkoxyalkylamine, alkoxyoxycarboxylate, amino, alkylamino, or dialkylamino.

In some embodiments of any one of the aspect, R^{3NN} is hydrogen, hydroxyl, protected hydroxyl, halogen, optionally substituted C₁₋₃₀ alkoxy, or alkoxyalkyl (e.g., methoxy, 2-methoxyethoxy, dimethylaminoethoxyethyoxy, N-methylmethoxyamido).

In some embodiments of any one of the aspects, R^{3NN} is hydrogen, hydroxyl, protected hydroxyl, methoxy, 2-methoxyethoxy, —O-dimethylaminoethoxyethyl (—O-DMAEOE) or —O—N-methylacetamido (—O-NMA).

In some embodiments of any one of the aspects, R^{3NN} is hydrogen, hydroxyl, protected hydroxyl or methoxy.

In some embodiments of any one of the aspects, R^{3NN} is a reactive phosphorous group. Optionally, only one of R² and R³ is a reactive phosphorous group.

In some embodiments of any one of the aspects descried herein, R^{3NN} is a solid support or a linker covalently attached to a solid support. For example, R³ is —OC(O)CH₂CH₂C(O)NH—Z, where Z is a solid support. Optionally, only one of R² and R³ is a solid support or a linker covalently attached to a solid support.

In some embodiments of any one of the aspects, when R^{3NN} is —OR³³², R³³² can be hydrogen or a hydroxyl protecting group. For example, R³³² can be hydrogen in some embodiments of any one of the aspects described herein. In some embodiments, R³ is —OC(O)CH₂CH₂CO₂H.

When R^{3NN} is —SR³³³, R³³³ can be hydrogen or a sulfur protecting group. Accordingly, in some embodiments of any one of the aspects, R³³³ is hydrogen.

When R^{3NN} is —O(CH₂CH₂O)ᵣCH₂CH₂OR³³⁴, r can be 1-50; R³³⁴ is independently for each occurrence H, C₁-C₃₀alkyl, cyclyl, heterocyclyl, aryl, heteroaryl, aralkyl, sugar or R³³⁵; and R³³⁵ is independently for each occurrence amino (NH₂), alkylamino, dialkylamino, arylamino, diarylamino, heteroarylamino, or diheteroaryl amino.

When R^{3NN} is —NH(CH₂CH₂NH)ₛCH₂CH₂—R³³⁵, s can be 1-50 and R³³⁵ can be independently for each occurrence amino (NH₂), alkylamino, dialkylamino, arylamino, diarylamino, heteroarylamino, or diheteroaryl amino.

In some embodiments of any one of the aspects described herein, R^{3NN} is hydrogen, halogen, —OR³³², or optionally substituted C₁-C₃₀alkoxy. For example, R³ is halogen, —OR³³², or optionally substituted C₁-C₃₀alkoxy. In some embodiments of any one of the aspects described herein, R^{3NN} is F, OH or optionally substituted C₁-C₃₀alkoxy.

In some embodiments of any one of the aspects described herein, R^{3NN} is C₁-C₃₀alkoxy optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)— alkyl, C(O)— alkyl, alkylcarbonylaminyl, CH₂—[CH(OH)]ₘ—(CH₂)ₚ-OH, CH₂—[CH(OH)]ₘ—(CH₂)ₚ—NH₂ or CH₂-aryl-alkoxy, where “m” and “p” are independently 1, 2, 3, 4, 5 or 6. For example, R^{3NN} is C₁-C₃₀alkoxy optionally substituted with a NH₂, OH, C(O)NH₂, COOH, halo, SH, or C₁-C₆alkoxy. In some embodiments of any one of the aspects described herein, R³ is —O(CH₂)ₜCH₃, where t is 1-21.

In some embodiments of any one of the aspects, R^{3NN} is —O(CH₂)ᵤR³³⁷, where u is 2-10; R³³⁷ is C₁-C₆alkoxy, amino (NH₂), CO₂H, OH or halo. For example, R³³⁷ is —CH₃ or NH₂. Accordingly, in some embodiments of any one of the aspects described herein, R^{3NN} is —O(CH₂)ᵤ—OMe or R^{3NN} is —O(CH₂)ᵤNH₂

In some embodiments of any one of the aspects described herein, u is 2, 3, 4, 5 or 6. For example, u is 2, 3 or 6. In one non-limiting example, u is 2. In another non-limiting example, u is 3 or 6.

In some embodiments of any one of the aspects described herein, R^{3NN} is a C₁-C₆haloalkyl. For example, R^{3NN} is a C₁-C₄haloalkyl. In some embodiments of any one of the aspects described herein, R³ is —CF₃, —CF₂CF₃, —CF₂CF₂CF₃ or —CF₂(CF₃)₂.

In some embodiments of any one of the aspects described herein, R^{3NN} is —OCH(CH₂OR³³⁸)CH₂OR³³⁹, where R³³⁸ and R³³⁹ independently are H, optionally substituted C₁-C₃₀alkyl, optionally substituted C₂-C₃₀alkenyl or optionally substituted C₂-C₃₀alkynyl. For example, R³³⁸ and R³³⁹ independently are optionally substituted C₁-C₃₀alkyl.

In some embodiments of any one of the aspects described herein, R^{3NN} is —CH₂C(O)NHR³³¹⁰, where R³³¹⁰ is H, optionally substituted C₁-C₃₀alkyl, optionally substituted C₂-C₃₀alkenyl or optionally substituted C₂-C₃₀alkynyl.

### R^{5NN}

In some embodiments of the various aspects described herein, R^{5N} is R⁶, —Z-L²-R⁶, R⁵⁵¹, hydrogen, hydroxyl, optionally substituted C₁₋₆alkyl-R⁵⁵¹, optionally substituted —C₂₋₆alkenyl-R⁵⁵, or optionally substituted —C₂₋₆alkynyl-R⁵⁵¹, where R⁵⁵¹ can be —OR⁵⁵², —SR⁵⁵³, hydrogen, a phosphorous group, a solid support or a linker to a solid support. When R⁵⁵¹ is —OR⁵², R⁵⁵² can be H or a hydroxyl protecting group. Similarly, when R⁵⁵¹ is —SR⁵⁵³, R⁵⁵³ can be H or a sulfur protecting group.

In some embodiments of any one of the aspects described herein, R^{5NN} is —OR⁵⁵² or —SR⁵⁵³.

In some embodiments of any one of the aspects described herein, R^{5′} is —OR⁵⁵² and R⁵⁵² is 4,4′-dimethoxytrityl (DMT), e.g., R^{5NN} is —O-DMT.

In some embodiments of any one of the aspects described herein, R⁵¹ is —CH(R⁵⁵⁴)—R⁵⁵¹, where R⁵⁵⁴ is hydrogen, halogen, optionally substituted C₁-C₃₀alkyl, optionally substituted C₂-C₃₀alkenyl, optionally substituted C₂-C₃₀alkynyl, or optionally substituted C₁-C₃₀alkoxy.

In some embodiments of any one of the aspects, when R⁵ is —CH(R⁵⁵⁴)—R, R⁵⁵⁴ is H or C₁-C₃₀alkyl optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)— alkyl, C(O)— alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy, where “m” and “p” are independently 1, 2, 3, 4, 5 or 6. For example, R⁵⁵⁴ is H. In some other non-limiting examples, R⁵⁵⁴ is C₁-C₃₀alkyl optionally substituted with a NH₂, OH, C(O)NH₂, COOH, halo, SH, or C₁-C₆alkoxy.

In some embodiments of the various aspects described herein, R^{5NN} is —CH(R⁵⁵⁴)—O—R⁵⁵², where R⁵⁵⁴ is H or C₁-C₃₀alkyl optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)-alkyl, C(O)— alkyl, alkylcarbonylaminyl, CH₂—[CH(OH)]ₘ—(CH₂)ₚ—OH, CH₂—[CH(OH)]ₘ—(CH₂)ₚ-NH₂ or CH₂-aryl-alkoxy, where “m” and “p” are independently 1, 2, 3, 4, 5 or 6. For example, R⁵⁵⁴ is H. In some other non-limiting examples, R⁵⁵⁴ is C₁-C₃₀alkyl optionally substituted with a NH₂, OH, C(O)NH₂, COOH, halo, SH, or C₁-C₆alkoxy.

In some embodiments of the various aspects described herein, R^{5′} is optionally substituted C₁₋₆alkyl-R⁵⁵¹ or optionally substituted —C₂₋₆alkenyl-R⁵⁵¹,

In some embodiments of any one of the aspects described herein, R^{5′} is —C(R⁵⁵⁴)=CHR⁵⁵¹. It is noted that the double bond in —C(R⁵⁵⁴)=CHR⁵⁵¹ can be in the cis or trans configuration. Accordingly, in some embodiments of any one of the aspects, R^{d} is —C(R⁵⁵⁴)=CHR⁵⁵¹ and wherein the double bond is in the cis configuration. In some other embodiments of any one of the aspects, R^{d} is —C(R⁵⁵⁴)=CHR⁵⁵¹ and wherein the double bond is in the trans configuration.

In some embodiments of any one of the aspects described herein, R^{5NN} is —CH═CHR⁵⁵¹.

In some embodiments of any one of the aspects, when R^{5NN} is —C(R⁵⁵⁴)=CHR⁵⁵¹, R⁵⁵⁴ is H or C₁-C₃₀alkyl optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)— alkyl, C(O)— alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy, where “m” and “p” are independently 1, 2, 3, 4, 5 or 6; and R⁵⁵¹ is a phosphorous group. For example, R⁵ is —CH═CHR⁵⁵¹.

In some embodiments of any one of the aspects described herein, R⁵⁵¹ is a reactive phosphorous group.

In some embodiments of any one of the aspects, R^{5NN} is —CH═CH—P(O)(OR⁵⁵⁵)₂, —CH═CH—P(S)(OR⁵⁵⁵)₂, —CH═CH—P(S)(SR⁵⁵⁶)(OR⁵⁵⁵), —CH═CH—P(S)(SR⁵⁵⁶)₂, —CH═CH—OP(O)(OR⁵⁵⁵)₂, —CH═CH—OP(S)(OR⁵⁵⁵)₂, —CH═CH—OP(S)(SR⁵⁵⁶)(OR⁵⁵⁵), —CH═CH—OP(S)(SR⁵⁵⁶)₂, —CH═CH—SP(O)(OR⁵⁵⁵)₂, —CH═CH—SP(S)(OR⁵⁵⁵)₂, —CH═CH—SP(S)(SR⁵⁵⁶)(OR⁵⁵), or —CH═CH—SP(S)(SR⁵⁵⁶)₂, where each R⁵⁵⁵ is independently hydrogen, optionally substituted C₁₋₃₀alkyl, optionally substituted C₂₋₃₀alkenyl, or optionally substituted C₂₋₃₀alkynyl, or an oxygen-protecting group; and each R⁵⁵⁶ is independently hydrogen, optionally substituted C₁₋₃₀alkyl, optionally substituted C₂₋₃₀alkenyl, or optionally substituted C₂₋₃₀alkynyl, or a sulfur-protecting group.

In some embodiments of any one of the aspects, at least one R⁵⁵⁵ in —P(O)(OR⁵⁵⁵)₂, —P(S)(OR⁵⁵⁵)₂, —P(S)(SR⁵⁵⁶)(OR⁵⁵⁵), —OP(O)(OR⁵⁵⁵)₂, —OP(S)(OR⁵⁵⁵)₂, —OP(S)(SR⁵⁵⁶)(OR⁵⁵⁵), SP(O)(OR⁵⁵⁵)₂, —SP(S)(OR⁵⁵⁵)₂, and —SP(S)(SR⁵⁵⁶)(OR⁵⁵⁵) is hydrogen.

In some other embodiments of any one of the aspects, at least one R⁵⁵⁵ in —P(O)(OR⁵⁵⁵)₂, —P(S)(OR⁵⁵⁵)₂, —P(S)(SR⁵⁵⁶)(OR⁵⁵⁵), —OP(O)(OR⁵⁵⁵)₂, —OP(S)(OR⁵⁵⁵)₂, —OP(S)(SR⁵⁵⁶)(OR⁵⁵⁵), SP(O)(OR⁵⁵⁵)₂, —SP(S)(OR⁵⁵⁵)₂, or —SP(S)(SR⁵⁵⁶)(OR⁵⁵⁵) is not hydrogen. For example, at least one at least one R⁵⁵⁵ in P(O)(OR⁵⁵⁵)₂, —P(S)(OR⁵⁵⁵)₂, —P(S)(SR⁵⁵⁶)(OR⁵⁵⁵), —OP(O)(OR⁵⁵⁵)₂, —OP(S)(OR⁵⁵⁵)₂, —OP(S)(SR⁵⁵⁶)(OR⁵⁵⁵), SP(O)(OR⁵⁵⁵)₂, —SP(S)(OR⁵⁵⁵)₂, and —SP(S)(SR⁵⁵⁶)(OR⁵⁵⁵) is optionally substituted C₁₋₃₀alkyl, optionally substituted C₂₋₃₀alkenyl, or optionally substituted C₂₋₃₀alkynyl, or an oxygen-protecting group.

In some embodiments of any one of the aspects, at least one R⁵⁵⁵ is H and at least one R⁵⁵⁵ is other than H in —P(O)(OR⁵⁵⁵)₂, —P(S)(OR⁵⁵)₂, —P(S)(SR⁵⁵⁶)(OR⁵⁵), —OP(O)(OR⁵⁵)₂, —OP(S)(OR⁵⁵⁵)₂, —OP(S)(SR⁵⁵⁶)(OR⁵⁵⁵), SP(O)(OR⁵⁵⁵)₂, —SP(S)(OR⁵⁵⁵)₂, and —SP(S)(SR⁵⁵⁶)(OR⁵⁵⁵).

In some embodiments of any one of the aspects, all R⁵⁵⁵ are H in —P(O)(OR⁵⁵⁵)₂, —P(S)(OR⁵⁵⁵)₂, —P(S)(SR⁵⁵⁶)(OR⁵⁵⁵), —OP(O)(OR⁵⁵⁵)₂, —OP(S)(OR⁵⁵⁵)₂, —OP(S)(SR⁵⁵⁶)(OR⁵⁵⁵), —OP(S)(SR⁵⁵⁶)₂, —SP(O)(OR⁵⁵⁵)₂, —SP(S)(OR⁵⁵⁵)₂, —SP(S)(SR⁵⁵⁶)(OR⁵⁵⁵), and —SP(S)(SR⁵⁵⁶)₂.

In some embodiments of any one of the aspects, all R⁵⁵⁵ are other than H in in —P(O)(OR⁵⁵⁵)₂, —P(S)(OR⁵⁵⁵)₂, —P(S)(SR⁵⁵⁶)(OR⁵⁵⁵), —OP(O)(OR⁵⁵⁵)₂, —OP(S)(OR⁵⁵⁵)₂, —OP(S)(SR⁵⁵⁶)(OR⁵⁵⁵), —OP(S)(SR⁵⁵⁶)₂, —SP(O)(OR⁵⁵⁵)₂, —SP(S)(OR⁵⁵⁵)₂, —SP(S)(SR⁵⁵⁶)(OR⁵⁵⁵), and —SP(S)(SR⁵⁵⁶)₂.

In some embodiments of any one of the aspects, at least one R⁵⁵⁶ in —P(S)(SR⁵⁵⁶)(OR⁵⁵⁵), —P(S)(SR⁵⁵⁶)₂, —OP(S)(OR⁵⁵⁵)₂, —OP(S)(SR⁵⁵⁶)(OR⁵⁵⁵), —OP(S)(SR⁵⁵⁶)₂, —SP(S)(SR⁵⁵⁶)(OR⁵⁵⁵), and —SP(S)(SR⁵⁵⁶)₂ is H.

In some embodiments of any one of the aspects, at least one R⁵⁵⁶ in —P(S)(SR⁵⁵⁶)(OR⁵⁵⁵), —P(S)(SR⁵⁵⁶)₂, —OP(S)(OR⁵⁵⁵)₂, —OP(S)(SR⁵⁵⁶)(OR⁵⁵⁵), —OP(S)(SR⁵⁵⁶)₂, —SP(S)(SR⁵⁵⁶)(OR⁵⁵⁵), and —SP(S)(SR⁵⁵⁶)₂ is other than H. For example, at least one R⁵⁵⁶ in —P(S)(SR⁵⁵⁶)(OR⁵⁵⁵), —P(S)(SR⁵⁵⁶)₂, —OP(S)(OR⁵⁵⁵)₂, —OP(S)(SR⁵⁵⁶)(OR⁵⁵⁵), —OP(S)(SR⁵⁵⁶)₂, —SP(S)(SR⁵⁵⁶)(OR⁵⁵⁵), and —SP(S)(SR⁵⁵⁶)₂ is optionally substituted C₁₋₃₀alkyl, optionally substituted C₂₋₃₀alkenyl, or optionally substituted C₂₋₃₀alkynyl, or an sulfur-protecting group.

In some embodiments of any one of the aspects, at least one R⁵⁵⁶ is H and at least one R⁵⁵⁶ is other than H in —P(S)(SR⁵⁵⁶)₂, —OP(S)(SR⁵⁵⁶)₂ and —SP(S)(SR⁵⁵⁶)₂.

In some embodiments, all R⁵⁵⁶ are H in —P(S)(SR⁵⁵⁶)(OR⁵⁵⁵), —P(S)(SR⁵⁵⁶)₂, —OP(S)(OR⁵⁵⁵)₂, —OP(S)(SR⁵⁵⁶)(OR⁵⁵⁵), —OP(S)(SR⁵⁵⁶)₂, —SP(S)(SR⁵⁵⁶)(OR⁵⁵⁵), and —SP(S)(SR⁵⁵⁶)₂.

In some embodiments, all R⁵⁵⁶ are other than H in —P(S)(SR⁵⁵⁶)(OR⁵⁵⁵), —P(S)(SR⁵⁵⁶)₂, —OP(S)(OR⁵⁵⁵)₂, —OP(S)(SR⁵⁵⁶)(OR⁵⁵⁵), —OP(S)(SR⁵⁵⁶)₂, —SP(S)(SR⁵⁵⁶)(OR⁵⁵⁵), and —SP(S)(SR⁵⁵⁶)₂.

In some embodiments of any one of the aspects, R^{3NN} is a reactive phosphorous group, a solid support, a linker to a solid support, and R^{5NN} is a protected hydroxyl.

### L³

In the various aspects described herein, L³ can be a linker. For example, L³ can be a bond. In some embodiments of any one of the aspects described herein, L³ is an optionally substituted C₁-C₂₀alkylene, (e.g., —(CH₂)_{b}—, where b is 1, 2, 3, 4, 5, 6, 7, 8, 9, 10, 11, 12, 13, 14, 14, 15, 16, 17, 18, 19 or 20), or optionally substituted C₂-C₂₀alkynylene, and where the backbone of the alkylene or alkynylene can be interrupted or terminated by O, S, S(O), SO₂, NR¹, NR¹—C(O), C(O), C(O)O, cleavable linking group, substituted or unsubstituted aryl, substituted or unsubstituted heteroaryl, substituted or unsubstituted heterocyclic; where R^{N1} is hydrogen, acyl, aliphatic or substituted aliphatic. For example, L³ is an optionally substituted C₈-C₁₆alkylene, where the backbone of the alkylene can be interrupted or terminated by O, S, S(O), SO₂, NR¹, NR¹—C(O), C(O), or C(O)O. In some embodiments of any one of the aspects described herein, L³ is —C(O)—(CH₂)₁₀—, —C(O)—(CH₂)₁₁—, or —C(O)—(CH₂)₅NHC(O)—(CH₂)₁₁—.

### R^{AO}

In the various aspects described herein, each R^{AO} is independently —O-L³-R⁶.

In some embodiments of any one of the aspects, R^{AO} is —O-L³-R⁶. For example, R^{AO} is —O-L³-R⁶, where L³ is optionally substituted C₁-C₂₀alkylene, optionally substituted C₂-C₂₀alkenylene or optionally substituted C₂-C₂₀alkynylene, and where the backbone of the alkylene, alkenylene or alkynylene can be interrupted or terminated by O, S, S(O), SO₂, NR¹, NR¹—C(O), C(O), C(O)O, cleavable linking group, substituted or unsubstituted aryl, substituted or unsubstituted heteroaryl, substituted or unsubstituted heterocyclic; where R^{N1} is hydrogen, acyl, aliphatic or substituted aliphatic.

In some embodiments of any one of the aspects, R^{AO} is —O-L³-R⁶, where L³ is a polyethylene glycol (PEG).

In some embodiments of any one of the aspects, R^{AO} is —O-L³-R⁶, where R⁶ is —O—N(R⁷)R^{7′} or —O—N═C(R⁷)R^{7′}. For example, R^{AO} is —O-L³-O—N(R⁷)R^{7′} or —O-L³-O—N═C(R⁷)R^{7′}, where L³ is optionally substituted C₁-C₂₀alkylene, optionally substituted C₂-C₂₀alkenylene or optionally substituted C₂-C₂₀alkynylene, and where the backbone of the alkylene, alkenylene or alkynylene can be interrupted or terminated by O, S, S(O), SO₂, NR¹, NR¹—C(O), C(O), C(O)O, cleavable linking group, substituted or unsubstituted aryl, substituted or unsubstituted heteroaryl, substituted or unsubstituted heterocyclic; where R^{N1} is hydrogen, acyl, aliphatic or substituted aliphatic.

In some embodiments of any one of the aspects, R^{AO} is —O-L³-R⁶, where R⁶ is —O—N(R⁷)R^{7′}, where R⁷ and R^{7′} are independently H or a ligand, (e.g., a ligand selected independently from the group consisting of carbohydrates, lipids, vitamins, peptides, proteins, lipoproteins, peptidomimetics, polyamines, nucleosides and nucleotides, oligonucleotides, therapeutic agents, diagnostic agents, detectable labels, antibodies or fragments thereof, optionally substituted C₁₋₃₀ alkyl, optionally substituted C₁₋₃₀ alkenyl, optionally substituted C₁₋₃₀alkynyl, optionally substituted cycloalkyl, optionally substituted heterocyclyl, optionally substituted aryl, optionally substituted heteroaryl, polyethylene glycols (PEGs), nitrogen protecting group.

In some embodiments of any one of the aspects, R^{AO} is —O-L³-R⁶, where R⁶ is —O—N(R⁷)R^{7′}, where R⁷ and R^{7′} together with the N they are attached to form an optionally substituted heterocyclyl (e.g., phthalimide or morpholine).

In some embodiments of any one of the aspects, R^{AO} is —O-L³-R⁶, where R⁶ is —O—N═C(R⁷)R⁷.

### R³²

In some embodiments of any one of the aspects described herein, R³² is hydrogen, hydroxyl, halogen, protected hydroxyl, phosphate group, a reactive phosphorous group, optionally substituted C₁₋₃₀ alkyl, optionally substituted C₁₋₃₀haloalkyl, optionally substituted C₂₋₃₀alkenyl, optionally substituted C₂₋₃₀alkynyl, optionally substituted C₁₋₃₀ alkoxy (e.g., methoxy, 2-methoxyethoxy, dimethylaminoethoxyethyoxy, N-methylmethoxyamido), alkoxyalkyl (e.g., methoxyethyl), alkoxyalkylamine, alkoxyoxycarboxylate, amino, alkylamino, dialkylamino-O—C₄-30alkyl-ON(CH₂R⁸)(CH₂R⁹), —O—C₄₋₃₀alkyl-ON(CH₂R⁸)(CH₂R⁹), a bond to an internucleotide linkage to a subsequent nucleotide, a 3′-oligonuclotide capping group, a ligand, a solid support, a linker, a linker covalently bonded (e.g., —OC(O)CH₂CH₂C(O)—) to a solid support, or —Z-L²-R⁶.

In some embodiments of any one of the aspects described herein, R³² is a bond to an internucleotide linkage to a subsequent nucleotide, a 3′-oligonuclotide capping group (e.g., an inverted nucleotide or an inverted abasic nucleotide), a solid support, or a linker covalently bonded (e.g., —C(O)CH₂CH₂C(O)—) to a solid support. For example, R³² is a bond to an internucleotide linkage to a subsequent nucleotide, a solid support, or a linker covalently bonded (e.g., —C(O)CH₂CH₂C(O)—) to a solid support.

In some embodiments of any one of the aspects described herein, R³² is a bond to an internucleotide linkage to a subsequent nucleotide. It is noted that only one of R³² and R³³ can be a bond to an internucleotide linkage to a subsequent nucleotide.

In some embodiments of any one of the aspects descried herein, R³² is a solid support or a linker covalently attached to a solid support. For example, R³² is —OC(O)CH₂CH₂C(O)NH—Z, where Z is a solid support. In some embodiments, R³² is —OC(O)CH₂CH₂CO₂H. It is noted that only one of R³² and R³³ can be a solid support or a linker covalently attached to a solid support.

In some embodiments of any one of the aspects described herein, R³² is —Z-L²-R⁶, hydrogen, halogen, —OR³²², —SR³²³, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, amino (NH₂), alkylamino, dialkylamino, heterocyclyl, arylamino, diarylamino, heteroarylamino, diheteroarylamino, amino acid, —O(CH₂CH₂O)ᵣCH₂CH₂OR³²⁴, cyano, alkyl-thio-alkyl, thioalkoxy, cycloalkyl, aryl, heteroaryl, —NH(CH₂CH₂NH)ₛCH₂CH₂—R³²⁵, NHC(O)R³²⁴.

In some embodiments of any one of the aspects described herein, R³² is —Z-L²-R⁶, hydrogen, hydroxyl, protected hydroxyl, halogen, optionally substituted C₁₋₃₀ alkyl, optionally substituted C₂₋₃₀alkenyl, optionally substituted C₂₋₃₀alkynyl, optionally substituted C₁₋₃₀ alkoxy, alkoxyalkyl (e.g., methoxy, 2-methoxyethoxy, dimethylaminoethoxyethyoxy, N-methylmethoxyamido), alkoxyalkylamine, alkoxyoxycarboxylate, amino, alkylamino, dialkylamino, —O—C₄₋₃₀alkyl-ON(CH₂R⁸)(CH₂R⁹), or —O—C₄₋₃₀alkyl-ON(CH₂R⁸)(CH₂R⁹). For example, R³² is hydrogen, hydroxyl, protected hydroxyl, halogen, optionally substituted C₁₋₃₀ alkoxy, alkoxyalkyl (e.g., methoxy, 2-methoxyethoxy, dimethylaminoethoxyethyoxy, N-methylmethoxyamido), alkoxyalkylamine, alkoxyoxycarboxylate, amino, alkylamino, or dialkylamino.

In some embodiments of any one of the aspect, R³² is —Z-L²-R⁶, hydrogen, hydroxyl, protected hydroxyl, halogen, optionally substituted C₁₋₃₀ alkoxy, or alkoxyalkyl (e.g., methoxy, 2-methoxyethoxy, dimethylaminoethoxyethyoxy, N-methylmethoxyamido).

In some embodiments of any one of the aspects, R³² is —Z-L²-R⁶, hydrogen, hydroxyl, protected hydroxyl, fluoro, methoxy, 2-methoxyethoxy, —O-dimethylaminoethoxyethyl (—O-DMAEOE) or —O—N-methylacetamido (—O-NMA).

In some embodiments of any one of the aspects, R³² is —Z-L²-R⁶, hydrogen, hydroxyl, protected hydroxyl, fluoro, methoxy, 2-methoxyethoxy, —O-dimethylaminoethoxyethyl (—O-DMAEOE) or —O—N-methylacetamido (—O-NMA).

In some embodiments of any one of the aspects, R³² is hydrogen, hydroxyl, protected hydroxyl, fluoro, methoxy, 2-methoxyethoxy, —O-dimethylaminoethoxyethyl (—O-DMAEOE) or —O—N-methylacetamido (—O-NMA).

In some embodiments of any one of the aspects, R³² is —Z-L²-R⁶. For example, R³² is —Z-L²-R⁶, where Z is O.

In some embodiments of any one of the aspects, R³² is —Z-L²-R⁶. For example, R³² is —Z-L²-R⁶, where L² is optionally substituted C₁-C₂₀alkylene, optionally substituted C₂-C₂₀alkenylene or optionally substituted C₂-C₂₀alkynylene, and where the backbone of the alkylene, alkenylene or alkynylene can be interrupted or terminated by O, S, S(O), SO₂, NR¹, NR¹—C(O), C(O), C(O)O, cleavable linking group, substituted or unsubstituted aryl, substituted or unsubstituted heteroaryl, substituted or unsubstituted heterocyclic; where R^{N1} is hydrogen, acyl, aliphatic or substituted aliphatic.

In some embodiments of any one of the aspects, R³² is —Z-L²-R⁶, where L² is a polyethylene glycol (PEG).

In some embodiments of any one of the aspects, R³² is —Z-L²-R⁶, where Z is O and L² is optionally substituted C₁-C₂₀alkylene, optionally substituted C₂-C₂₀alkenylene or optionally substituted C₂-C₂₀alkynylene, and where the backbone of the alkylene, alkenylene or alkynylene can be interrupted or terminated by O, S, S(O), SO₂, NR¹, NR¹—C(O), C(O), C(O)O, cleavable linking group, substituted or unsubstituted aryl, substituted or unsubstituted heteroaryl, substituted or unsubstituted heterocyclic; where R^{N1} is hydrogen, acyl, aliphatic or substituted aliphatic.

In some embodiments of any one of the aspects, R³² is —Z-L²-R⁶, where Z is O and L² is a polyethylene glycol (PEG).

In some embodiments of any one of the aspects, R³² is —Z-L²-R⁶, where R⁶ is —O—N(R⁷)R^{7′} or —O—N═C(R⁷)R^{7′}. For example, R³² is —O-L²-O—N(R⁷)R^{7′} or —O-L²-O—N═C(R⁷)R^{7′}, where L² is optionally substituted C₁-C₂₀alkylene, optionally substituted C₂-C₂₀alkenylene or optionally substituted C₂-C₂₀alkynylene, and where the backbone of the alkylene, alkenylene or alkynylene can be interrupted or terminated by O, S, S(O), SO₂, NR¹, NR¹—C(O), C(O), C(O)O, cleavable linking group, substituted or unsubstituted aryl, substituted or unsubstituted heteroaryl, substituted or unsubstituted heterocyclic; where R^{N1} is hydrogen, acyl, aliphatic or substituted aliphatic.

In some embodiments of any one of the aspects, R³² is —Z-L²-R⁶, where R⁶ is —O—N(R⁷)R^{7′}. For example, R³² is —Z-L²-R⁶, where R⁶ is —O—N(R⁷)R^{7′}, where R⁷ and R^{7′} are independently H or a ligand, (e.g., a ligand selected independently from the group consisting of carbohydrates, lipids, vitamins, peptides, proteins, lipoproteins, peptidomimetics, polyamines, nucleosides and nucleotides, oligonucleotides, therapeutic agents, diagnostic agents, detectable labels, antibodies or fragments thereof, optionally substituted C₁₋₃₀ alkyl, optionally substituted C₁₋₃₀ alkenyl, optionally substituted C₁₋₃₀alkynyl, optionally substituted cycloalkyl, optionally substituted heterocyclyl, optionally substituted aryl, optionally substituted heteroaryl, polyethylene glycols (PEGs), nitrogen protecting group.

In some embodiments of any one of the aspects, R³² is —Z-L²-R⁶, where R⁶ is —O—N(R⁷)R^{7′}, where R⁷ and R^{7′} together with the N they are attached to form an optionally substituted heterocyclyl (e.g., phthalimide or morpholine).

In some embodiments of any one of the aspects, R³² is —Z-L²-R⁶, where R⁶ is —O—N═C(R⁷)R⁷.

In some embodiments of any one of the aspects described herein, R³² and R⁴ taken together are 4′-C(R¹⁰R¹¹)ᵥ—Y-2′ or 4′-Y—C(R¹⁰R¹¹)ᵥ-2′; v is 1, 2 or 3; where Y is —O—, —CH₂—, —CH(Me)-, —C(CH₃)₂—, —S—, —N(R¹²)—, —C(O)—, —C(S)—, —S(O)—, —S(O)₂—, —OC(O)—, —C(O)O—, —N(R¹²)C(O)—, or —C(O)N(R¹²)—; R¹⁰ and R¹¹ independently are H, optionally substituted C₁-C₆alkyl, optionally substituted C₂-C₆alkenyl or optionally substituted C₂-C₆alkynyl; R¹² is hydrogen, optionally substituted C₁₋₃₀alkyl, optionally substituted C₁-C₃₀alkoxy, C₁₋₄haloalkyl, optionally substituted C₂₋₄alkenyl, optionally substituted C₂₋₄alkynyl, optionally substituted C₁₋₃₀alky-CO₂H, or a nitrogen-protecting group.

In some embodiments of any one of the aspects, R³² and R⁴ taken together are 4′-C(R¹⁰R¹¹)ᵥ—O-2′.

In some embodiments of any one of the aspects, R³² and R⁴ taken together are 4′-CH₂-0-2′, 4′-CH(CH₃)—O-2′, 4′-CH(CH₂OCH₃)—O-2′, or 4′-CH₂CH₂—O-2′. For example, R³² and R⁴ taken together are 4′-CH₂CH₂—O-2′.

In some embodiments of any one of the aspects, when R³² is —OR³²², R³²² can be hydrogen or a hydroxyl protecting group. When R³² is —SR³²³, R³²³ can be hydrogen or a sulfur protecting group. Accordingly, in some embodiments of any one of the aspects, R³²³ is hydrogen.

When R³² is —O(CH₂CH₂O)ᵣCH₂CH₂OR³²⁴, r can be 1-50; R³²⁴ is independently for each occurrence H, C₁-C₃₀alkyl, cyclyl, heterocyclyl, aryl, heteroaryl, aralkyl, sugar or R³²⁵; and R³²⁵ is independently for each occurrence amino (NH₂), alkylamino, dialkylamino, arylamino, diarylamino, heteroarylamino, or diheteroaryl amino.

When R³² is —NH(CH₂CH₂NH)ₛCH₂CH₂—R³²⁵, s can be 1-50 and R³²⁵ can be independently for each occurrence amino (NH₂), alkylamino, dialkylamino, arylamino, diarylamino, heteroarylamino, or diheteroaryl amino.

In some embodiments of any one of the aspects described herein, R³² is hydrogen, halogen, —OR³²², or optionally substituted C₁-C₃₀alkoxy. For example, R³² is halogen, —OR³²², or optionally substituted C₁-C₃₀alkoxy. In some embodiments of any one of the aspects described herein, R³² is F, OH or optionally substituted C₁-C₃₀alkoxy.

In some embodiments of any one of the aspects described herein, R³² is C₁-C₃₀alkoxy optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)— alkyl, C(O)— alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy, where “m” and “p” are independently 1, 2, 3, 4, 5 or 6. For example, R³² is C₁-C₃₀alkoxy optionally substituted with a NH₂, OH, C(O)NH₂, COOH, halo, SH, or C₁-C₆alkoxy. In some embodiments of any one of the aspects described herein, R² is —O(CH₂)ₜCH₃, where t is 1-21. For example, t is 14, 15, 16, 17 or 18. In one non-limiting example, t is 16.

In some embodiments of any one of the aspects, R³² is —O(CH₂)ᵤR³²⁷, where u is 2-10; R³²⁷ is C₁-C₆alkoxy, amino (NH₂), CO₂H, OH or halo. For example, R³²⁷ is —CH₃ or NH₂. Accordingly, in some embodiments of any one of the aspects described herein, R³² is —O(CH₂)ᵤ—OMe or R³² is —O(CH₂)ᵤNH₂

In some embodiments of any one of the aspects described herein, R³² is a C₁-C₆haloalkyl. For example, R³² is a C₁-C₄haloalkyl. In some embodiments of any one of the aspects described herein, R³² is —CF₃, —CF₂CF₃, —CF₂CF₂CF₃ or —CF₂(CF₃)₂.

In some embodiments of any one of the aspects described herein, R³² is —OCH(CH₂OR³²⁸)CH₂OR³²⁹, where R³²⁸ and R³²⁹ independently are H, optionally substituted C₁-C₃₀alkyl, optionally substituted C₂-C₃₀alkenyl or optionally substituted C₂-C₃₀alkynyl. For example, R³²⁸ and R³²⁹ independently are optionally substituted C₁-C₃₀alkyl.

In some embodiments of any one of the aspects described herein, R³² is —CH₂C(O)NHR³²¹⁰, where R³²¹⁰ is H, optionally substituted C₁-C₃₀alkyl, optionally substituted C₂-C₃₀alkenyl or optionally substituted C₂-C₃₀alkynyl. For example, R³²¹⁰ is H or optionally substituted C₁-C₃₀alkyl. In some embodiments, R³²¹⁰ is optionally substituted C₁-C₆alkyl.

### R³³

In some embodiments of any one of the aspects described herein, R³³ is hydrogen, hydroxyl, halogen, protected hydroxyl, phosphate group, a reactive phosphorous group, optionally substituted C₁₋₃₀ alkyl, optionally substituted C₁₋₃₀haloalkyl, optionally substituted C₂₋₃₀alkenyl, optionally substituted C₂₋₃₀alkynyl, optionally substituted C₁₋₃₀ alkoxy (e.g., methoxy, 2-methoxyethoxy, dimethylaminoethoxyethyoxy, N-methylmethoxyamido), alkoxyalkyl (e.g., methoxyethyl), alkoxyalkylamine, alkoxyoxycarboxylate, amino, alkylamino, dialkylamino-O—C₄-30alkyl-ON(CH₂R⁸)(CH₂R⁹), —O—C₄₋₃₀alkyl-ON(CH₂R⁸)(CH₂R⁹), a bond to an internucleotide linkage to a subsequent nucleotide, a 3′-oligonuclotide capping group, a ligand, a solid support, a linker, a linker covalently bonded (e.g., —OC(O)CH₂CH₂C(O)—) to a solid support, or —Z-L²-R⁶.

In some embodiments of any one of the aspects described herein, R³³ is a bond to an internucleotide linkage to a subsequent nucleotide, a 3′-oligonuclotide capping group (e.g., an inverted nucleotide or an inverted abasic nucleotide), a solid support, or a linker covalently bonded (e.g., —C(O)CH₂CH₂C(O)—) to a solid support. For example, R³³ is a bond to an internucleotide linkage to a subsequent nucleotide, a solid support, or a linker covalently bonded (e.g., —C(O)CH₂CH₂C(O)—) to a solid support.

In some embodiments of any one of the aspects described herein, R³³ is a bond to an internucleotide linkage to a subsequent nucleotide.

In some embodiments of any one of the aspects descried herein, R³³ is a solid support or a linker covalently attached to a solid support. For example, R³³ is —OC(O)CH₂CH₂C(O)NH—Z, where Z is a solid support. In some embodiments, R³³ is —OC(O)CH₂CH₂CO₂H.

In some embodiments of any one of the aspects described herein, R³³ is —Z-L²-R⁶, hydrogen, halogen, —OR³²², —SR³²³, optionally substituted C₁₋₃₀alkyl, C₁₋₃₀haloalkyl, optionally substituted C₂₋₃₀alkenyl, optionally substituted C₂₋₃₀alkynyl, or optionally substituted C₁₋₃₀alkoxy, amino (NH₂), alkylamino, dialkylamino, heterocyclyl, arylamino, diarylamino, heteroarylamino, diheteroarylamino, amino acid, —O(CH₂CH₂O)ᵣCH₂CH₂OR³²⁴, cyano, alkyl-thio-alkyl, thioalkoxy, cycloalkyl, aryl, heteroaryl, —NH(CH₂CH₂NH)ₛCH₂CH₂—R³²⁵, NHC(O)R³²⁴.

In some embodiments of any one of the aspects described herein, R³³ is —Z-L²-R⁶, hydrogen, hydroxyl, protected hydroxyl, optionally substituted C₁₋₃₀ alkoxy (e.g., methoxy, 2-methoxyethoxy, dimethylaminoethoxyethyoxy, N-methylmethoxyamido), alkoxyalkyl, alkoxyalkylamine, alkoxyoxycarboxylate, amino, alkylamino, dialkylamino, —O—C₄₋₃₀alkyl-ON(CH₂R⁸)(CH₂R⁹), or —O—C₄₋₃₀alkyl-ON(CH₂R⁸)(CH₂R⁹). For example, R³³ is hydrogen, hydroxyl, protected hydroxyl, optionally substituted C₁₋₃₀ alkoxy (e.g., methoxy, 2-methoxyethoxy, dimethylaminoethoxyethyoxy, N-methylmethoxyamido), alkoxyalkyl, alkoxyalkylamine, alkoxyoxycarboxylate, amino, alkylamino, or dialkylamino.

In some embodiments of any one of the aspect, R³³ is —Z-L²-R⁶, hydroxyl, protected hydroxyl, or optionally substituted C₁₋₃₀ alkoxy, (e.g., methoxy, 2-methoxyethoxy, dimethylaminoethoxyethyoxy, N-methylmethoxyamido).

In some embodiments of any one of the aspects, R³³ is —Z-L²-R⁶, hydroxyl, protected hydroxyl, methoxy, 2-methoxyethoxy, —O-dimethylaminoethoxyethyl (—O-DMAEOE) or —O—N-methylacetamido (—O-NMA).

In some embodiments of any one of the aspects, R³³ is —Z-L²-R⁶. For example, R³³ is —Z-L²-R⁶, where Z is O.

In some embodiments of any one of the aspects, R³³ is —Z-L²-R⁶. For example, R³³ is —Z-L²-R⁶, where L² is optionally substituted C₁-C₂₀alkylene, optionally substituted C₂-C₂₀alkenylene or optionally substituted C₂-C₂₀alkynylene, and where the backbone of the alkylene, alkenylene or alkynylene can be interrupted or terminated by O, S, S(O), SO₂, NR¹, NR¹—C(O), C(O), C(O)O, cleavable linking group, substituted or unsubstituted aryl, substituted or unsubstituted heteroaryl, substituted or unsubstituted heterocyclic; where R^{N1} is hydrogen, acyl, aliphatic or substituted aliphatic.

In some embodiments of any one of the aspects, R³³ is —Z-L²-R⁶, where L² is a polyethylene glycol (PEG).

In some embodiments of any one of the aspects, R³³ is —Z-L²-R⁶, where Z is O and L² is optionally substituted C₁-C₂₀alkylene, optionally substituted C₂-C₂₀alkenylene or optionally substituted C₂-C₂₀alkynylene, and where the backbone of the alkylene, alkenylene or alkynylene can be interrupted or terminated by O, S, S(O), SO₂, NR¹, NR¹—C(O), C(O), C(O)O, cleavable linking group, substituted or unsubstituted aryl, substituted or unsubstituted heteroaryl, substituted or unsubstituted heterocyclic; where R^{N1} is hydrogen, acyl, aliphatic or substituted aliphatic.

In some embodiments of any one of the aspects, R³³ is —Z-L²-R⁶, where Z is O and L² is a polyethylene glycol (PEG).

In some embodiments of any one of the aspects, R³³ is —Z-L²-R⁶, where R⁶ is —O—N(R⁷)R^{7′} or —O—N═C(R⁷)R^{7′}. For example, R³² is —O-L²-O—N(R⁷)R^{7′} or —O-L²-O—N═C(R⁷)R^{7′}, where L² is optionally substituted C₁-C₂₀alkylene, optionally substituted C₂-C₂₀alkenylene or optionally substituted C₂-C₂₀alkynylene, and where the backbone of the alkylene, alkenylene or alkynylene can be interrupted or terminated by O, S, S(O), SO₂, NR¹, NR¹—C(O), C(O), C(O)O, cleavable linking group, substituted or unsubstituted aryl, substituted or unsubstituted heteroaryl, substituted or unsubstituted heterocyclic; where R^{N1} is hydrogen, acyl, aliphatic or substituted aliphatic.

In some embodiments of any one of the aspects, R³³ is —Z-L²-R⁶, where R⁶ is —O—N(R⁷)R^{7′}. For example, R³³ is —Z-L²-R⁶, where R⁶ is —O—N(R⁷)R^{7′}, where R⁷ and R^{7′} are independently H or a ligand, (e.g., a ligand selected independently from the group consisting of carbohydrates, lipids, vitamins, peptides, proteins, lipoproteins, peptidomimetics, polyamines, nucleosides and nucleotides, oligonucleotides, therapeutic agents, diagnostic agents, detectable labels, antibodies or fragments thereof, optionally substituted C₁₋₃₀ alkyl, optionally substituted C₁₋₃₀ alkenyl, optionally substituted C₁₋₃₀alkynyl, optionally substituted cycloalkyl, optionally substituted heterocyclyl, optionally substituted aryl, optionally substituted heteroaryl, polyethylene glycols (PEGs), nitrogen protecting group.

In some embodiments of any one of the aspects, R³³ is —Z-L²-R⁶, where R⁶ is —O—N(R⁷)R^{7′}, where R⁷ and R^{7′} together with the N they are attached to form an optionally substituted heterocyclyl (e.g., phthalimide or morpholine).

In some embodiments of any one of the aspects, R³³ is —Z-L²-R⁶, where R⁶ is —O—N═C(R⁷)R⁷.

In some embodiments of any one of the aspects described herein, R³³ and R⁴ taken together are 4′-C(R¹⁰R¹¹)ᵥ—Y-2′ or 4′-Y—C(R¹⁰R¹¹)ᵥ-2′; v is 1, 2 or 3; where Y is —O—, —CH₂—, —CH(Me)-, —C(CH₃)₂—, —S—, —N(R¹²)—, —C(O)—, —C(S)—, —S(O)—, —S(O)₂—, —OC(O)—, —C(O)O—, —N(R¹²)C(O)—, or —C(O)N(R¹²)—; R¹⁰ and R¹¹ independently are H, optionally substituted C₁-C₆alkyl, optionally substituted C₂-C₆alkenyl or optionally substituted C₂-C₆alkynyl; R¹² is hydrogen, optionally substituted C₁₋₃₀alkyl, optionally substituted C₁-C₃₀alkoxy, C₁₋₄haloalkyl, optionally substituted C₂₋₄alkenyl, optionally substituted C₂₋₄alkynyl, optionally substituted C₁₋₃₀alky-CO₂H, or a nitrogen-protecting group.

In some embodiments of any one of the aspects, R³² and R⁴ taken together are 4′-C(R¹⁰R¹¹)ᵥ—O-2′.

In some embodiments of any one of the aspects, R³² and R⁴ taken together are 4′-CH₂-0-2′, 4′-CH(CH₃)—O-2′, 4′-CH(CH₂OCH₃)—O-2′, or 4′-CH₂CH₂—O-2′. For example, R³² and R⁴ taken together are 4′-CH₂CH₂—O-2′.

### R^{33M}

In some embodiments of any one of the aspects described herein, R^{33M} is hydrogen, hydroxyl, halogen, protected hydroxyl, phosphate group, a reactive phosphorous group, optionally substituted C₁₋₃₀ alkyl, optionally substituted C₁₋₃₀haloalkyl, optionally substituted C₂₋₃₀alkenyl, optionally substituted C₂₋₃₀alkynyl, optionally substituted C₁₋₃₀ alkoxy (e.g., methoxy, 2-methoxyethoxy, dimethylaminoethoxyethyoxy, N-methylmethoxyamido), alkoxyalkyl (e.g., methoxyethyl), alkoxyalkylamine, alkoxyoxycarboxylate, amino, alkylamino, dialkylamino-O—C₄-30alkyl-ON(CH₂R⁸)(CH₂R⁹), —O—C₄₋₃₀alkyl-ON(CH₂R⁸)(CH₂R⁹), a bond to an internucleotide linkage to a subsequent nucleotide, a 3′-oligonuclotide capping group, a ligand, a solid support, a linker, a linker covalently bonded (e.g., —OC(O)CH₂CH₂C(O)—) to a solid support.

In some embodiments of any one of the aspects described herein, R^{33M} is a bond to an internucleotide linkage to a subsequent nucleotide, a 3′-oligonuclotide capping group (e.g., an inverted nucleotide or an inverted abasic nucleotide), a solid support, or a linker covalently bonded (e.g., —C(O)CH₂CH₂C(O)—) to a solid support. For example, R^{33M} is a bond to an internucleotide linkage to a subsequent nucleotide, a solid support, or a linker covalently bonded (e.g., —C(O)CH₂CH₂C(O)—) to a solid support.

In some embodiments of any one of the aspects described herein, R^{33M} is a bond to an internucleotide linkage to a subsequent nucleotide.

In some embodiments of any one of the aspects descried herein, R^{33M} is a solid support or a linker covalently attached to a solid support. For example, R^{33M} is —OC(O)CH₂CH₂C(O)NH—Z, where Z is a solid support. In some embodiments, R^{33M} is —OC(O)CH₂CH₂CO₂H.

In some embodiments of any one of the aspects described herein, R^{33M} is —Z—R^{33L}. For example, R^{33M} is —O—R^{33L}.

In some embodiments of any one of the aspects described herein, R^{33L} is a ligand or a linker covalently linked to one or more ligands.

In some embodiments of any one of the aspects described herein, R^{33L} is optionally substituted C₁₋₃₀ alkyl, optionally substituted C₂₋₃₀alkenyl, optionally substituted C₂₋₃₀alkynyl, or polyethylene glycol.

In some embodiments of any one of the aspects described herein, R^{33L} is alkyl, alkyl ester, mono-GalNac, Tri-GalNac, hetero-alky, alkyne, alkene, alkylether, PEG, Biotin, Vit-E, alkyl folate, cyclic RGD.

In some embodiments of any one of the aspects described herein, R^{333L} is a peptide. Some exemplary peptides as source of aminooxy amine are described in Mezoe et al., J. Pept. Sci. 2011, 17, 39; Jimenez-Castells et al., Bioorg. Med. Chem. Lett. 2007, 17, 5155; and Lee et al., Synlett 2003, 325, contents of all which are incorporated herein by reference in their entireties.

### R³⁵

In some embodiments of the various aspects described herein, R³⁵ is R⁶, —Z-L²-R⁶, a bond to an internucleotide linkage to a preceding nucleotide, hydrogen, hydroxyl, protected hydroxyl, phosphate group, optionally substituted C₁₋₃₀ alkyl, optionally substituted C₁₋₃₀haloalkyl, optionally substituted C₂₋₃₀alkenyl, optionally substituted C₂₋₃₀alkynyl, optionally substituted C₁₋₃₀ alkoxy, halogen, alkoxyalkyl (e.g., methoxyethyl), alkoxyalkylamine, alkoxyoxycarboxylate, amino, alkylamino, dialkylamino, —O—C₄₋₃₀alkyl-ON(CH₂R⁸)(CH₂R⁹), —O—C₄₋₃₀alkyl-ON(CH₂R⁸)(CH₂R⁹), vinylphosphonate (VP) group, C₃₋₆cycloalkylphosphonate (e.g., cyclopropylphosphonate), monophosphate ((HO)₂(O)P—O-5′), diphosphate ((HO)₂(O)P—O—P(HO)(O)—O-5′), triphosphate ((HO)₂(O)P—O—(HO)(O)P—O—P(HO)(O)—O-5′); monothiophosphate (phosphorothioate, (HO)₂(S)P—O-5′), monodithiophosphate (phosphorodithioate; (HO)(HS)(S)P—O-5′), phosphorothiolate ((HO)₂(O)P—S-5′); alpha-thiotriphosphate; beta-thiotriphosphate; gamma-thiotriphosphate; phosphoramidates ((HO)₂(0)P—NH-5′, (HO)(NH₂)(O)P—O-5′), alkylphosphonates (R(OH)(O)P—O-5′, R=alkyl, e.g., methyl, ethyl, isopropyl, propyl, etc. . . . ), alkyletherphosphonates (R(OH)(O)P—O-5′, R=alkylether, e.g., methoxymethyl (CH₂OMe), ethoxymethyl, etc. . . . ), (HO)₂(X)P—O[—(CH₂)ₐ—O—P(X)(OH)—O]_{b}-5′ or (HO)₂(X)P—O[—(CH₂)ₐ—P(X)(OH)—O]_{b}-5′ or (HO)₂(X)P—[—(CH₂)ₐ—O—P(X)(OH)—O]_{b}-5′, where X is O, S or optionally substituted alkyl, and dialkyl terminal phosphates and phosphate mimics (e.g., HO[—(CH₂)ₐ—O—P(X)(OH)—O]_{b}-5′, H₂N[—(CH₂)ₐ—O—P(X)(OH)—O]_{b}-5′, H[—(CH₂)ₐ—O—P(X)(OH)—O]_{b}-5′, Me₂N[—(CH₂)ₐ—O—P(X)(OH)—O]_{b}-5′, HO[—(CH₂)ₐ—P(X)(OH)—O]_{b}-5′, H₂N[—(CH₂)ₐ—P(X)(OH)—O]_{b}-5′, H[—(CH₂)ₐ—P(X)(OH)—O]_{b}-5′, Me₂N[—(CH₂)ₐ—P(X)(OH)—O]_{b}-5′, wherein X is O or S; and a and b are each independently 1-10).

In some embodiments of any one of the aspects, R³⁵ is a bond to an internucleotide linkage to a preceding nucleotide.

In some embodiments of any one of the aspects, R³⁵ is R⁶. For example, R³⁵ is —O—N(R⁷)R^{7′} or —O—N═C(R⁷)R^{7′}.

In some embodiments of any one of the aspects, R³⁵ is —O—N(R⁷)R^{7′}. For example, R³⁵ is —O—N(R⁷)R^{7′}, where R⁷ and R^{7′} are independently H or a ligand, (e.g., a ligand selected independently from the group consisting of carbohydrates, lipids, vitamins, peptides, proteins, lipoproteins, peptidomimetics, polyamines, nucleosides and nucleotides, oligonucleotides, therapeutic agents, diagnostic agents, detectable labels, antibodies or fragments thereof, optionally substituted C₁₋₃₀ alkyl, optionally substituted C₁₋₃₀ alkenyl, optionally substituted C₁₋₃₀alkynyl, optionally substituted cycloalkyl, optionally substituted heterocyclyl, optionally substituted aryl, optionally substituted heteroaryl, polyethylene glycols (PEGs), nitrogen protecting group.

In some embodiments of any one of the aspects, R³⁵ is —O—N(R⁷)R^{7′}, where R⁷ and R^{7″} together with the N they are attached to form an optionally substituted heterocyclyl (e.g., phthalimide or morpholine).

In some embodiments of any one of the aspects, R³⁵ is —O—N═C(R⁷)R^{7′}.

In some embodiments of any one of the aspects, R³⁵ is —Z-L²-R⁶. For example, R⁵ is —Z-L²-R⁶, where Z is O.

In some embodiments of any one of the aspects, R³⁵ is —Z-L²-R⁶. For example, R⁵ is —Z-L²-R⁶, where L² is optionally substituted C₁-C₂₀alkylene, optionally substituted C₂-C₂₀alkenylene or optionally substituted C₂-C₂₀alkynylene, and where the backbone of the alkylene, alkenylene or alkynylene can be interrupted or terminated by O, S, S(O), SO₂, NR¹, NR¹—C(O), C(O), C(O)O, cleavable linking group, substituted or unsubstituted aryl, substituted or unsubstituted heteroaryl, substituted or unsubstituted heterocyclic; where R^{N1} is hydrogen, acyl, aliphatic or substituted aliphatic.

In some embodiments of any one of the aspects, R³⁵ is —Z-L²-R⁶, where L² is a polyethylene glycol (PEG).

In some embodiments of any one of the aspects, R³⁵ is —Z-L²-R⁶, where Z is O and L² is optionally substituted C₁-C₂₀alkylene, optionally substituted C₂-C₂₀alkenylene or optionally substituted C₂-C₂₀alkynylene, and where the backbone of the alkylene, alkenylene or alkynylene can be interrupted or terminated by O, S, S(O), SO₂, NR¹, NR¹—C(O), C(O), C(O)O, cleavable linking group, substituted or unsubstituted aryl, substituted or unsubstituted heteroaryl, substituted or unsubstituted heterocyclic; where R^{N1} is hydrogen, acyl, aliphatic or substituted aliphatic.

In some embodiments of any one of the aspects, R³⁵ is —Z-L²-R⁶, where Z is O and L² is a polyethylene glycol (PEG).

In some embodiments of any one of the aspects, R³⁵ is —Z-L²-R⁶, where R⁶ is —O—N(R⁷)R^{7′} or —O—N═C(R⁷)R^{7′}. For example, R³⁵ is —O-L²-O—N(R⁷)R^{7′} or —O-L²-O—N═C(R⁷)R⁷, where L² is optionally substituted C₁-C₂₀alkylene, optionally substituted C₂-C₂₀alkenylene or optionally substituted C₂-C₂₀alkynylene, and where the backbone of the alkylene, alkenylene or alkynylene can be interrupted or terminated by O, S, S(O), SO₂, NR¹, NR¹—C(O), C(O), C(O)O, cleavable linking group, substituted or unsubstituted aryl, substituted or unsubstituted heteroaryl, substituted or unsubstituted heterocyclic; where R^{N1} is hydrogen, acyl, aliphatic or substituted aliphatic.

In some embodiments of any one of the aspects, R³⁵ is —Z-L²-R⁶, where R⁶ is —O—N(R⁷)R^{7′}. For example, R³⁵ is —Z-L²-R⁶, where R⁶ is —O—N(R⁷)R^{7′}, where R⁷ and R^{7′} are independently H or a ligand, (e.g., a ligand selected independently from the group consisting of carbohydrates, lipids, vitamins, peptides, proteins, lipoproteins, peptidomimetics, polyamines, nucleosides and nucleotides, oligonucleotides, therapeutic agents, diagnostic agents, detectable labels, antibodies or fragments thereof, optionally substituted C₁₋₃₀ alkyl, optionally substituted C₁₋₃₀ alkenyl, optionally substituted C₁₋₃₀alkynyl, optionally substituted cycloalkyl, optionally substituted heterocyclyl, optionally substituted aryl, optionally substituted heteroaryl, polyethylene glycols (PEGs), nitrogen protecting group.

In some embodiments of any one of the aspects, R³⁵ is —Z-L²-R⁶, where R⁶ is —O—N(R⁷)R^{7′}, where R⁷ and R^{7′} together with the N they are attached to form an optionally substituted heterocyclyl (e.g., phthalimide or morpholine).

In some embodiments of any one of the aspects, R³⁵ is —Z-L²-R⁶, where R⁶ is —O—N═C(R⁷)R⁷.

In some embodiments of any one of the aspects described herein, R³⁵ is —OR⁵⁵² or —SR⁵⁵³. For example, R³⁵ is —OR⁵⁵² and R⁵⁵² is 4,4′-dimethoxytrityl (DMT), e.g., R³⁵ is —O-DMT.

In some embodiments of any one of the aspects described herein, R³⁵ is —CH(R⁵⁵⁴)—R⁵⁵¹, where R⁵⁵⁴ is hydrogen, halogen, optionally substituted C₁-C₃₀alkyl, optionally substituted C₂-C₃₀alkenyl, optionally substituted C₂-C₃₀alkynyl, or optionally substituted C₁-C₃₀alkoxy.

In some embodiments of any one of the aspects, when R³⁵ is —CH(R⁵⁵⁴)—R⁵⁵¹, R⁵⁵⁴ is H or C₁-C₃₀alkyl optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)— alkyl, C(O)— alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy, where “m” and “p” are independently 1, 2, 3, 4, 5 or 6. For example, R⁵⁵⁴ is H. In some other non-limiting examples, R⁵⁵⁴ is C₁-C₃₀alkyl optionally substituted with a NH₂, OH, C(O)NH₂, COOH, halo, SH, or C₁-C₆alkoxy.

In some embodiments of the various aspects described herein, R³⁵ is —CH(R⁵⁵⁴)—O—R⁵⁵² where R⁵⁵⁴ is H or C₁-C₃₀alkyl optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)— alkyl, C(O)— alkyl, alkylcarbonylaminyl, CH₂—[CH(OH)]ₘ—(CH₂)ₚ—OH, CH₂—[CH(OH)]ₘ—(CH₂)ₚ-NH₂ or CH₂-aryl-alkoxy, where “m” and “p” are independently 1, 2, 3, 4, 5 or 6. For example, R⁵⁵⁴ is H. In some other non-limiting examples, R⁵⁵⁴ is C₁-C₃₀alkyl optionally substituted with a NH₂, OH, C(O)NH₂, COOH, halo, SH, or C₁-C₆alkoxy.

In some embodiments of the various aspects described herein, R³⁵ is optionally substituted C₁₋₆alkyl-R⁵⁵¹ or optionally substituted —C₂₋₆alkenyl-R⁵⁵¹,

In some embodiments of any one of the aspects, R³⁵ is —C(R⁵⁵⁴)=CHR⁵⁵¹ and wherein the double bond is in the cis configuration. In some other embodiments of any one of the aspects, R³⁵ is —C(R⁵⁵⁴)=CHR⁵⁵¹ and wherein the double bond is in the trans configuration.

In some embodiments of any one of the aspects described herein, R³⁵ is —CH═CHR⁵¹.

In some embodiments of any one of the aspects, when R³⁵ is —C(R⁵⁵⁴)=CHR⁵⁵¹, R⁵⁵⁴ is H or C₁-C₃₀alkyl optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)— alkyl, C(O)— alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy, where “m” and “p” are independently 1, 2, 3, 4, 5 or 6; and R⁵⁵¹ is a phosphorous group. For example, R³⁵ is —CH═CHR⁵⁵¹.

In some embodiments of any one of the aspects, R³⁵ is —CH═CH—P(O)(OR⁵⁵⁵)₂, —CH═CH—P(S)(OR⁵⁵⁵)₂, —CH═CH—P(S)(SR⁵⁵⁶)(OR⁵⁵⁵), —CH═CH—P(S)(SR⁵⁵⁶)₂, —CH═CH—OP(O)(OR⁵⁵⁵)₂, —CH═CH—OP(S)(OR⁵⁵⁵)₂, —CH═CH—OP(S)(SR⁵⁵⁶)(OR⁵⁵⁵), —CH═CH—OP(S)(SR⁵⁵⁶)₂, —CH═CH—SP(O)(OR⁵⁵⁵)₂, —CH═CH—SP(S)(OR⁵⁵⁵)₂, —CH═CH—SP(S)(SR⁵⁵⁶)(OR⁵⁵), or —CH═CH—SP(S)(SR⁵⁵⁶)₂, where each R⁵⁵⁵ is independently hydrogen, optionally substituted C₁₋₃₀alkyl, optionally substituted C₂₋₃₀alkenyl, or optionally substituted C₂₋₃₀alkynyl, or an oxygen-protecting group; and each R⁵⁵⁶ is independently hydrogen, optionally substituted C₁₋₃₀alkyl, optionally substituted C₂₋₃₀alkenyl, or optionally substituted C₂₋₃₀alkynyl, or a sulfur-protecting group.

### R^{43N}

In some embodiments of any one of the aspects described herein, R^{43N} is hydrogen, hydroxyl, halogen, protected hydroxyl, phosphate group, a reactive phosphorous group, optionally substituted C₁₋₃₀ alkyl, optionally substituted C₁₋₃₀haloalkyl, optionally substituted C₂₋₃₀alkenyl, optionally substituted C₂₋₃₀alkynyl, optionally substituted C₁₋₃₀ alkoxy (e.g., methoxy, 2-methoxyethoxy, dimethylaminoethoxyethyoxy, N-methylmethoxyamido), alkoxyalkyl (e.g., methoxyethyl), alkoxyalkylamine, alkoxyoxycarboxylate, amino, alkylamino, dialkylamino-O—C₄-30alkyl-ON(CH₂R⁸)(CH₂R⁹), —O—C₄₋₃₀alkyl-ON(CH₂R⁸)(CH₂R⁹), a bond to an internucleotide linkage to a subsequent nucleotide, a 3′-oligonuclotide capping group, a ligand, a solid support, a linker, or a linker covalently bonded (e.g., —OC(O)CH₂CH₂C(O)—) to a solid support.

In some embodiments of any one of the aspects described herein, R^{43N} is a bond to an internucleotide linkage to a subsequent nucleotide, a 3′-oligonuclotide capping group (e.g., an inverted nucleotide or an inverted abasic nucleotide), a solid support, or a linker covalently bonded (e.g., —C(O)CH₂CH₂C(O)—) to a solid support. For example, R^{43N} is a bond to an internucleotide linkage to a subsequent nucleotide, a solid support, or a linker covalently bonded (e.g., —C(O)CH₂CH₂C(O)—) to a solid support.

In some embodiments of any one of the aspects described herein, R^{43N} is a bond to an internucleotide linkage to a subsequent nucleotide.

In some embodiments of any one of the aspects descried herein, R^{43N} is a solid support or a linker covalently attached to a solid support. For example, R^{43N} is —OC(O)CH₂CH₂C(O)NH—Z, where Z is a solid support. In some embodiments, R^{43N} is —OC(O)CH₂CH₂CO₂H.

### R^{45N}

In some embodiments of the various aspects described herein, R^{45N} is a bond to an internucleotide linkage to a preceding nucleotide, hydrogen, hydroxyl, protected hydroxyl, phosphate group, optionally substituted C₁₋₃₀ alkyl, optionally substituted C₁₋₃₀haloalkyl, optionally substituted C₂₋₃₀alkenyl, optionally substituted C₂₋₃₀alkynyl, optionally substituted C₁₋₃₀ alkoxy, halogen, alkoxyalkyl (e.g., methoxyethyl), alkoxyalkylamine, alkoxyoxycarboxylate, amino, alkylamino, dialkylamino, —O—C₄₋₃₀alkyl-ON(CH₂R⁸)(CH₂R⁹), —O—C₄₋₃₀alkyl-ON(CH₂R⁸)(CH₂R⁹), vinylphosphonate (VP) group, C₃₋₆cycloalkylphosphonate (e.g., cyclopropylphosphonate), monophosphate ((HO)₂(O)P—O-5′), diphosphate ((HO)₂(O)P—O—P(HO)(O)—O-5′), triphosphate ((HO)₂(O)P—O—(HO)(O)P—O—P(HO)(O)—O-5′); monothiophosphate (phosphorothioate, (HO)₂(S)P—O-5′), monodithiophosphate (phosphorodithioate; (HO)(HS)(S)P—O-5′), phosphorothiolate ((HO)2(O)P—S-5′); alpha-thiotriphosphate; beta-thiotriphosphate; gamma-thiotriphosphate; phosphoramidates ((HO)₂(O)P—NH-5′, (HO)(NH₂)(O)P—O-5′), alkylphosphonates (R(OH)(O)P—O-5′, R=alkyl, e.g., methyl, ethyl, isopropyl, propyl, etc. . . . ), alkyletherphosphonates (R(OH)(O)P—O-5′, R=alkylether, e.g., methoxymethyl (CH₂OMe), ethoxymethyl, etc. . . . ), (HO)₂(X)P—O[—(CH₂)ₐ—O—P(X)(OH)—O]_{b}-5′ or (HO)₂(X)P—O[—(CH₂)ₐ—P(X)(OH)—O]_{b}-5′ or (HO)₂(X)P—[—(CH₂)ₐ—O—P(X)(OH)—O]_{b}-5′, where X is O, S or optionally substituted alkyl, and dialkyl terminal phosphates and phosphate mimics (e.g., HO[—(CH₂)ₐ—O—P(X)(OH)—O]_{b}-5′, H2N[—(CH₂)ₐ—O—P(X)(OH)—O]_{b}-5′, H[—(CH₂)ₐ—O—P(X)(OH)—O]_{b}-5′, Me₂N[—(CH₂)ₐ—O—P(X)(OH)—O]_{b}-5′, HO[—(CH₂)ₐ—P(X)(OH)—O]_{b}-5′, H₂N[—(CH₂)ₐ—P(X)(OH)—O]_{b}-5′, H[—(CH₂)ₐ—P(X)(OH)—O]_{b}-5′, Me₂N[—(CH₂)ₐ—P(X)(OH)—O]_{b}-5′, wherein X is O or S; and a and b are each independently 1-10).

In some embodiments of any one of the aspects, R^{45N} is a bond to an internucleotide linkage to a preceding nucleotide.

In some embodiments of any one of the aspects described herein, R^{45N} is —OR⁵⁵² or —SR⁵⁵³. For example, R^{45N} is —OR⁵⁵² and R⁵⁵² is 4,4′-dimethoxytrityl (DMT), e.g., R^{45N} is —O-DMT.

In some embodiments of any one of the aspects described herein, R^{45N} is —CH(R⁵⁵⁴)—R⁵⁵¹, where R⁵⁵⁴ is hydrogen, halogen, optionally substituted C₁-C₃₀alkyl, optionally substituted C₂-C₃₀alkenyl, optionally substituted C₂-C₃₀alkynyl, or optionally substituted C₁-C₃₀alkoxy.

In some embodiments of any one of the aspects, when R^{45N} is —CH(R⁵⁵⁴)—R⁵⁵¹, R⁵⁵⁴ is H or C₁-C₃₀alkyl optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)— alkyl, C(O)— alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy, where “m” and “p” are independently 1, 2, 3, 4, 5 or 6. For example, R⁵⁵⁴ is H. In some other non-limiting examples, R⁵⁵⁴ is C₁-C₃₀alkyl optionally substituted with a NH₂, OH, C(O)NH₂, COOH, halo, SH, or C₁-C₆alkoxy.

In some embodiments of the various aspects described herein, R^{45N} is —CH(R⁵⁵⁴)—O—R⁵⁵², where R⁵⁵⁴ is H or C₁-C₃₀alkyl optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)-alkyl, C(O)— alkyl, alkylcarbonylaminyl, CH₂—[CH(OH)]ₘ—(CH₂)ₚ—OH, CH₂—[CH(OH)]ₘ—(CH₂)ₚ-NH₂ or CH₂-aryl-alkoxy, where “m” and “p” are independently 1, 2, 3, 4, 5 or 6. For example, R⁵⁵⁴ is H. In some other non-limiting examples, R¹⁵⁴ is C₁-C₃₀alkyl optionally substituted with a NH₂, OH, C(O)NH₂, COOH, halo, SH, or C₁-C₆alkoxy.

In some embodiments of the various aspects described herein, R^{45N} is optionally substituted C₁₋₆alkyl-R⁵⁵¹ or optionally substituted —C₂₋₆alkenyl-R⁵⁵¹,

In some embodiments of any one of the aspects, R^{45N} is —C(R⁵⁵⁴)=CHRW⁵⁵¹ and wherein the double bond is in the cis configuration. In some other embodiments of any one of the aspects, R³⁵ is —C(R⁵⁵⁴)=CHR⁵⁵¹ and wherein the double bond is in the trans configuration.

In some embodiments of any one of the aspects described herein, R^{45N} is —CH═CHR⁵⁵¹.

In some embodiments of any one of the aspects, when R³⁵ is —C(R⁵⁵⁴)=CHR⁵⁵¹, R⁵⁵⁴ is H or C₁-C₃₀alkyl optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)— alkyl, C(O)— alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy, where “m” and “p” are independently 1, 2, 3, 4, 5 or 6; and R⁵⁵¹ is a phosphorous group. For example, R^{45N} is —CH═CHR⁵⁵¹.

In some embodiments of any one of the aspects, R^{45N} is —CH═CH—P(O)(OR⁵⁵⁵)₂, —CH═CH—P(S)(OR⁵⁵⁵)₂, —CH═CH—P(S)(SR⁵⁵⁶)(OR⁵⁵⁵), —CH═CH—P(S)(SR⁵⁵⁶)₂, —CH═CH—OP(O)(OR⁵⁵⁵)₂, —CH═CH—OP(S)(OR⁵⁵⁵)₂, —CH═CH—OP(S)(SR⁵⁵⁶)(OR⁵⁵⁵), —CH═CH—OP(S)(SR⁵⁵⁶)₂, —CH═CH—SP(O)(OR⁵⁵⁵)₂, —CH═CH—SP(S)(OR⁵⁵⁵)₂, —CH═CH—SP(S)(SR⁵⁵⁶)(OR⁵⁵), or —CH═CH—SP(S)(SR⁵⁵⁶)₂, where each R⁵⁵⁵ is independently hydrogen, optionally substituted C₁₋₃₀alkyl, optionally substituted C₂₋₃₀alkenyl, or optionally substituted C₂₋₃₀alkynyl, or an oxygen-protecting group; and each R⁵⁵⁶ is independently hydrogen, optionally substituted C₁₋₃₀alkyl, optionally substituted C₂₋₃₀alkenyl, or optionally substituted C₂₋₃₀alkynyl, or a sulfur-protecting group.

### J

In compounds of Formulae Ig, Ii, IIg and IVg, J can be O, S, CH₂ or N-alkyl (e.g., NCH₃).

In some embodiments of any one of the aspects, J is O.

In some embodiments of any one of the aspects, J is S.

In some embodiments of any one of the aspects, J is CH₂.

In some embodiments of any one of the aspects, J s N-alkyl, where the alkyl can be optionally substituted with 1, 2, 3, 4 or 5 independently selected substituents. For example, J is N—C₁₋₆alkyl, where the C₁₋₆alkyl alkyl is optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)-alkyl, C(O)— alkyl, alkylcarbonylaminyl, CH₂—[CH(OH)]ₘ—(CH₂)ₚ—OH, CH₂—[CH(OH)]ₘ—(CH₂)ₚ-NH₂ or CH₂-aryl-alkoxy, where “m” and “p” are independently 1, 2, 3, 4, 5 or 6.

### Internucleoside Linkages

As used herein, “internucleoside linkage” refers to a covalent linkage between adjacent nucleosides. The two main classes of internucleoside linkages are defined by the presence or absence of a phosphorus atom. Representative phosphorus containing linkages include, but are not limited to, phosphodiesters (P═O), phosphotriesters, methylphosphonates, phosphoramidate, and phosphorothioates (P═S). Representative non-phosphorus containing linking groups include, but are not limited to, methylenemethylimino (—CH₂—N(CH₃)—O—CH₂—), thiodiester (—O—C(O)—S—), thionocarbamate (—O—C(O)(NH)—S—); siloxane (—O—Si(H)₂—O—); and N,N′-dimethylhydrazine (—CH₂—N(CH₃)—N(CH₃)—). Modified internucleoside linkages, compared to natural phosphodiester linkages, can be used to alter, typically increase, nuclease resistance of the oligonucleotide compound. In certain embodiments, linkages having a chiral atom can be prepared as racemic mixtures, as separate enantiomers. Representative chiral linkages include, but are not limited to, alkylphosphonates and phosphorothioates. Methods of preparation of phosphorous-containing and non-phosphorous-containing linkages are well known to those skilled in the art.

The phosphate group in the internucleoside linkage can be modified by replacing one of the oxygens with a different substituent. One result of this modification can be increased resistance of the oligonucleotide to nucleolytic breakdown. Examples of modified phosphate groups include phosphorothioate, phosphoroselenates, borano phosphates, borano phosphate esters, hydrogen phosphonates, phosphoroamidates, alkyl or aryl phosphonates and phosphotriesters. In some embodiments, one of the non-bridging phosphate oxygen atoms in the phosphodiester internucleoside linkage can be replaced by any of the following: S, Se, BR₃ (R is hydrogen, alkyl, aryl), C (i.e. an alkyl group, an aryl group, etc. . . . ), H, NR₂ (R is hydrogen, optionally substituted alkyl, aryl), or OR (R is optionally substituted alkyl or aryl). The phosphorous atom in an unmodified phosphate group is achiral. However, replacement of one of the non-bridging oxygens with one of the above atoms or groups of atoms renders the phosphorous atom chiral. In other words a phosphorous atom in a phosphate group modified in this way is a stereogenic center. The stereogenic phosphorous atom can possess either the “R” configuration (herein Rp) or the “S” configuration (herein Sp).

Phosphorodithioates have both non-bridging oxygens replaced by sulfur. The phosphorus center in the phosphorodithioates is achiral which precludes the formation of oligonucleotides diastereomers. Thus, while not wishing to be bound by theory, modifications to both non-bridging oxygens, which eliminate the chiral center, e.g. phosphorodithioate formation, can be desirable in that they cannot produce diastereomer mixtures. The non-bridging oxygens can be independently any one of O, S, Se, B, C, H, N, or OR (R is alkyl or aryl).

A phosphodiester internucleoside linkage can also be modified by replacement of bridging oxygen, (i.e. oxygen that links the phosphate to the sugar of the nucleosides), with nitrogen (bridged phosphoroamidates), sulfur (bridged phosphorothioates) and carbon (bridged methylenephosphonates). The replacement can occur at the either one of the linking oxygens or at both linking oxygens. When the bridging oxygen is the 3′-oxygen of a nucleoside, replacement with carbon is preferred. When the bridging oxygen is the 5′-oxygen of a nucleoside, replacement with nitrogen is preferred.

Modified phosphate linkages where at least one of the oxygen linked to the phosphate has been replaced or the phosphate group has been replaced by a non-phosphorous group, are also referred to as “non-phosphodiester intersugar linkage” or “non-phosphodiester linker.”

In certain embodiments, the phosphate group can be replaced by non-phosphorus containing connectors, e.g. dephospho linkers. Dephospho linkers are also referred to as non-phosphodiester linkers herein. While not wishing to be bound by theory, it is believed that since the charged phosphodiester group is the reaction center in nucleolytic degradation, its replacement with neutral structural mimics should impart enhanced nuclease stability. Again, while not wishing to be bound by theory, it can be desirable, in some embodiment, to introduce alterations in which the charged phosphate group is replaced by a neutral moiety.

Examples of moieties which can replace the phosphate group include, but are not limited to, amides (for example amide-3 (3′-CH₂—C(═O)—N(H)-5′) and amide-4 (3′-CH₂—N(H)—C(═O)-5′)), hydroxylamino, siloxane (dialkylsiloxane), carboxamide, carbonate, carboxymethyl, carbamate, carboxylate ester, thioether, ethylene oxide linker, sulfide, sulfonate, sulfonamide, sulfonate ester, thioformacetal (3′-S—CH₂—O-5′), formacetal (3′-O—CH₂—O-5′), oxime, methyleneimino, methykenecarbonylamino, methylenemethylimino (MMI, 3′-CH₂—N(CH₃)—O-5′), methylenehydrazo, methylenedimethylhydrazo, methyleneoxymethylimino, ethers (C3′-O—C5′), thioethers (C3′-S—C5′), thioacetamido (C3′-N(H)—C(═O)—CH₂—S—C5′, C3′-O—P(O)—O—SS—C5′, C3′—CH₂—NH—NH—C5′, 3′-NHP(O)(OCH₃)—O-5′ and 3′-NHP(O)(OCH₃)—O-5′ and nonionic linkages containing mixed N, O, S and CH₂ component parts. See for example, Carbohydrate Modifications in Antisense Research; Y. S. Sanghvi and P. D. Cook Eds. ACS Symposium Series 580; Chapters 3 and 4, (pp. 40-65). Preferred embodiments include methylenemethylimino (MMI), methylenecarbonylamino, amides, carbamate and ethylene oxide linker.

One skilled in the art is well aware that in certain instances replacement of a non-bridging oxygen can lead to enhanced cleavage of the intersugar linkage by the neighboring 2′-OH, thus in many instances, a modification of a non-bridging oxygen can necessitate modification of 2′-OH, e.g., a modification that does not participate in cleavage of the neighboring intersugar linkage, e.g., arabinose sugar, 2′-O-alkyl, 2′-F, LNA and ENA.

Preferred non-phosphodiester internucleoside linkages include phosphorothioates, phosphorothioates with an at least 1%, 5%, 10%, 20%, 30%, 40%, 50%, 60%, 70%, 80%, 90% 95% or more enantiomeric excess of Sp isomer, phosphorothioates with an at least 1%, 5%, 10%, 20%, 30%, 40%, 50%, 60%, 70%, 80%, 90% 95% or more enantiomeric excess of Rp isomer, phosphorodithioates, phsophotriesters, aminoalkylphosphotrioesters, alkyl-phosphonaters (e.g., methyl-phosphonate), selenophosphates, phosphoramidates (e.g., N-alkylphosphoramidate), and boranophosphonates.

Additional exemplary non-phosphorus containing internucleoside linking groups are described in U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439, content of each of which is incorporated herein by reference.

In some embodiments of any one of the aspects, the oligonucleotides described herein comprise one or more neutral internucleoside linkages that are non-ionic. Suitable neutral internucleoside linkages include, but are not limited to, phosphotriesters, methylphosphonates, MMI (3′-CH₂—N(CH₃)—O-5′), amide-3 (3′-CH₂—C(═O)—N(H)-5′), amide-4 (3′-CH₂—N(H)—C(═O)-5′), formacetal (3′-O—CH₂—O-5′), and thioformacetal (3′-S—CH₂—O-5′); nonionic linkages containing siloxane (dialkylsiloxane), carboxylate ester, carboxamide, sulfide, sulfonate ester and/or amides (See for example: Carbohydrate Modifications in Antisense Research; Y. S. Sanghvi and P. D. Cook Eds. ACS Symposium Series 580; Chapters 3 and 4, (pp. 40-65)); and nonionic linkages containing mixed N, O, S and CH₂ component parts.

In one embodiment, the non-phosphodiester backbone linkage is selected from the group consisting of phosphorothioate, phosphorodithioate, alkyl-phosphonate and phosphoramidate backbone linkages.

In some embodiments of any one of the aspects described herein, the internucleoside linkage is

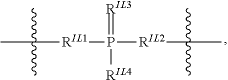

where R^{IL1} and R^{IL2} are each independently for each occurrence absent, O, S, CH₂, NR (R is hydrogen, alkyl, aryl), or optionally substituted alkylene, wherein backbone of the alkylene can comprise one or more of O, S, SS and NR (R is hydrogen, alkyl, aryl) internally and/or at the end; and R^{IL3} and R⁴ are each independently selected from the group consisting of O, OR (R is hydrogen, alkyl, aryl), S, Se, BR₃ (R is hydrogen, alkyl, aryl), BH₃—, C (i.e. an alkyl group, an aryl group, etc. . . . ), H, NR₂ (R is hydrogen, alkyl, aryl), alkyl or aryl. It is understood that one of R^{IL1} and R^{IL2} is replacing the oxygen linked to 5′ carbon of a first nucleoside sugar and the other of R^{IL1} and R^{IL2} is replacing the oxygen linked to 3′ (or 2′) carbon of a second nucleoside sugar.

In some embodiments of any one of the aspects, R^{IL1}, R^{IL2}, R^{IL3} and R⁴ all are O.

In some embodiments, R^{IL1} and R^{IL2} are O and at least one of R^{IL3} and R^{IL4} is other than 0. For example, one of R^{IL3} and R^{IL4} is S and the other is O or both of R^{IL3} and R⁴ are S.

In some embodiments of any one of the aspects described herein, one of R³³ or R³⁵ is a bond to a modified internucleoside linkage, e.g., an internucleoside linkage of structure:

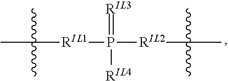

where at least one of R^{IL1}, R^{IL2}, R^{IL3} and R^{IL4} is not O. For example, at least one of R^{IL3} and R^{IL4} is S.

In some embodiments of any one of the aspects described herein, both of R³³ and R³⁵ are a bond to a modified internucleoside linkage.

In some embodiments of any one of the aspects described herein R³³ is a bond to phosphodiester internucleoside linkage.

In some embodiments of any one of the aspects described herein R³¹ is a bond to phosphodiester internucleoside linkage.

In some embodiments of any one of the aspects described herein, R³³ is a bond to a modified internucleoside linkage and R³⁵ is a bond to phosphodiester internucleoside linkage.

In some embodiments of any one of the aspects described herein, R³⁵ is a bond to a modified internucleoside linkage and R³³ is a bond to phosphodiester internucleoside linkage.

In some embodiments of any one of the aspects described herein, one of R^{33M} or R³⁵ is a bond to a modified internucleoside linkage, e.g., an internucleoside linkage of structure:

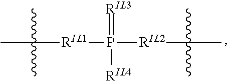

where at least one of R^{IL1}, R^{IL2}, R^{IL3} and R^{IL4} is not O. For example, at least one of R^{IL3} and R^{IL4} is S.

In some embodiments of any one of the aspects described herein, both of R^{33M} and R³⁵ are a bond to a modified internucleoside linkage.

In some embodiments of any one of the aspects described herein R^{33M} is a bond to phosphodiester internucleoside linkage.

In some embodiments of any one of the aspects described herein R³⁵ is a bond to phosphodiester internucleoside linkage.

In some embodiments of any one of the aspects described herein, R^{33M} is a bond to a modified internucleoside linkage and R³⁵ is a bond to phosphodiester internucleoside linkage.

In some embodiments of any one of the aspects described herein, R³⁵ is a bond to a modified internucleoside linkage and R^{33M} is a bond to phosphodiester internucleoside linkage.

In some embodiments of any one of the aspects described herein, one of R^{43N} or R^{45N} is a bond to a modified internucleoside linkage, e.g., an internucleoside linkage of structure:

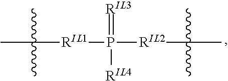

where at least one of R^{IL1}, R^{IL2}, R^{IL3} and R^{IL4} is not O. For example, at least one of R^{IL3} and R^{IL4} is S.

In some embodiments of any one of the aspects described herein, both of R^{43N} and R^{45N}are a bond to a modified internucleoside linkage.

In some embodiments of any one of the aspects described herein R^{43N} is a bond to phosphodiester internucleoside linkage.

In some embodiments of any one of the aspects described herein R^{45N} is a bond to phosphodiester internucleoside linkage.

In some embodiments of any one of the aspects described herein, R^{43N} is a bond to a modified internucleoside linkage and R^{45N} is a bond to phosphodiester internucleoside linkage.

In some embodiments of any one of the aspects described herein, R^{45N} is a bond to a modified internucleoside linkage and R^{43N} is a bond to phosphodiester internucleoside linkage.

In some embodiments of any one of the aspects, the oligonucleotide can comprise one or more, e.g., 1, 2, 3, 4, 5, 6, 7, 8 or more modified internucleoside linkages. For example, the oligonucleotide can comprise 1, 2, 3, 4, 5 or 6 modified internucleoside linkages. For example, the oligonucleotide comprises 1, 2, 3 or 4 modified internucleoside linkages. In some embodiments, the oligonucleotide comprises at least two modified internucleoside linkages between the first five nucleotides counting from the 5′-end of the oligonucleotide and further comprises at least two modified internucleoside linkages between the first five nucleotides counting from the 3′-end of the oligonucleotide. For example, the oligonucleotide comprises modified internucleoside linkages between nucleotides 1 and 2, and between nucleotides 2 and 3, counting from 5′-end of the oligonucleotide, and between nucleotides 1 and 2, and between nucleotides 2 and 3, counting from 3′-end of the oligonucleotide.

In some embodiments of any one of the aspects, the modified internucleoside linkage is a phosphorothioate. Accordingly, in some embodiments of any one of the aspects, the oligonucleotide comprises one or more, e.g., 1, 2, 3, 4, 5, 6, 7, 8 or more phosphorothioate internucleoside linkages. For example, the oligonucleotide comprises 1, 2, 3, 4, 5 or 6 phosphorothioate internucleoside linkages. For example, the oligonucleotide comprises 1, 2, 3 or 4 phosphorothioate internucleoside linkages. In some embodiments, the oligonucleotide comprises at least two phosphorothioate internucleoside linkages between the first five nucleotides counting from the 5′-end of the oligonucleotide and further comprises at least two phosphorothioate internucleoside linkages between the first five nucleotides counting from the 3′-end of the oligonucleotide. For example, the oligonucleotide comprises modified internucleoside linkages between nucleotides 1 and 2, and between nucleotides 2 and 3, counting from 5′-end of the oligonucleotide, and between nucleotides 1 and 2, and between nucleotides 2 and 3, counting from 3′-end of the oligonucleotide.

### Oxygen Protecting Groups

Some embodiments of the various aspects described herein include an oxygen protecting group (also referred to as an hydroxyl protecting group herein). Oxygen protecting groups include, but are not limited to, —R^{OP1}, —N(R^{OP2})₂, —C(═O)SR^{OP1}, —C(═O)R^{OP1}, —CO₂R^{OP1}, —C(═O)N(R^{OP2})₂, —C(═NR^{OP2})R^{OP1}, —C(═NR^{OP2})OR^{OP1}, —C(═NR^{OP2})N(R^{OP2})₂, —S(═O)R^{OP1}, —SO⁺₂R^{OP1}, —Si(R^{OP1})₃, —P(R^{OP3})₂, —P(R^{OP3})+3 X⁻, —P(OR^{OP3})₂, —P(OR^{OP3})₃ X⁻, —P(═O)(R^{OP1})₂, —P(═O)(OR^{OP3})₂, and —P(═O)(N(R^{OP2})₂)₂; wherein each X⁻ is a counterion; each R^{OP1} is independently C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀alkenyl, heteroC₂₋₁₀alkynyl, C₃₋₁₀ carbocyclyl, 3-14 membered heterocyclyl, C₆₋₁₄ aryl, or 5-14 membered heteroaryl, or two R^{OP1} groups are joined to form a 3-14 membered heterocyclyl or 5-14 membered heteroaryl ring; each R^{OP2} is hydrogen, —OH, —OR^{OP1}, —N(R^{OP3})₂, —CN, —C(═O)R^{OP1}, —C(═O)N(R^{OP3})₂, —CO₂R^{OP1}, —SO₂R^{OP1}, —C(═NR^{OP3})OR^{OP1}, —C(═NR^{OP3})N(R^{OP3})₂, —SO₂N(R^{OP3})₂, —SO₂R^{OP3}, —SO₂OR^{OP3}, SOR^{OP1}, —C(═S)N(R^{OP3})₂, —C(═O)SR^{OP3}, —C(═S)SR^{OP3}, —P(═O)(R^{OP1})₂, —P(═O)(OR^{OP3})₂, —P(═O)(N(R^{OP3})₂)₂, C₁₋₁₀ alkyl, C₁₋₁₀ perhaloalkyl, C₂₋₁₀ alkenyl, C₂₋₁₀ alkynyl, heteroC₁₋₁₀alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{OP2} groups are joined to form a 3-14 membered heterocyclyl or 5-14 membered heteroaryl ring; and each R^{OP3} is independently hydrogen, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{OP3} groups are joined to form a 3-14 membered heterocyclyl or 5-14 membered heteroaryl ring; and wherein each alkyl, alkenyl, alkynyl, carbocyclyl, heterocyclyl, aralkyl, aryl, and heteroaryl of R^{OP1}, R^{OP2} and R^{OP3} can be optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)— alkyl, C(O)— alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy, where “m” and “p” are independently 1, 2, 3, 4, 5 or 6.

Oxygen protecting groups are well known in the art and include those described in detail in Greene's Protecting Groups in Organic Synthesis, P. G. M. Wuts, 5^{th} Edition, John Wiley & Sons, 2014, incorporated herein by reference.

Exemplary oxygen protecting groups include, but are not limited to, methyl, t-butyloxycarbonyl (BOC or Boc), methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodisulfuran-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), adamantoate, crotonate, 4-methoxycrotonate, benzoate, p-phenylbenzoate, 2,4,6-trimethylbenzoate (mesitoate), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuSP3inoate, (E)-2-methyl-2-butenoate, o-(methoxyacyl)benzoate, α-naphthoate, nitrate, alkylN,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In some embodiments of any one of the aspects described herein, oxygen protecting group is benzyl, benzoyl, 2,6-dichlorobenzyl, t-butyldimethylsilyl, t-butyldiphenylsilyl, mesylate, tosylate, 4,4′-dimethoxytrityl (DMT), 9-phenylxanthine-9-yl (Pixyl) and 9-(p-methoxyphenyl)xanthine-9-yl (MOX). In certain embodiments, the hydroxyl protecting group is selected from acetyl, benzyl, t-butyldimethylsilyl, t-butyldiphenylsilyl and dimethoxytrityl wherein a more preferred hydroxyl protecting group is 4,4′-dimethoxytrityl.

The terms “protected hydroxy” and “protected hydroxyl” as used herein mean a group of the formula —OR^{Pro}, wherein R^{Pro} is an oxygen protecting group as defined herein.

### Nitrogen Protecting Groups

Some embodiments of the various aspects described herein include a nitrogen protecting group (also referred to as an amino protecting group herein). Nitrogen protecting groups include, but are not limited to, —OH, —OR^{NP1}, —N(R^{NP2})₂, —C(═O)R^{NP1}, —C(═O)N(R^{NP2})₂, —CO₂R^{NP1}, —SO₂R^{NP1}, —C(═NR^{NP2})RNP, —C(═NR^{NP2})OR^{NP1}, —C(═NR^{NP2})N(R^{NP2})₂, —SO₂N(R^{NP2})₂, —SO₂R^{NP2}, —SO₂OR^{NP2}, —SOR^{NP1}, —C(═S)N(R^{NP2})₂, —C(═O)SR^{NP2}, —C(═S)SR^{NP2}, C₁₋₁₀ alkyl (e.g., aralkyl, heteroaralkyl), C₂₋₁₀ alkenyl, C₂₋₁₀ alkynyl, C₃₋₁₀ carbocyclyl, 3-14 membered heterocyclyl, C₆-14 aryl, and 5-14 membered heteroaryl groups, where each R^{m} is independently C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀alkenyl, heteroC₂₋₁₀alkynyl, C₃₋₁₀ carbocyclyl, 3-14 membered heterocyclyl, C₆₋₁₄ aryl, or 5-14 membered heteroaryl, or two R^{NP1} groups are joined to form a 3-14 membered heterocyclyl or 5-14 membered heteroaryl ring; and each R^{NP2} is independently hydrogen, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{SP3} groups are joined to form a 3-14 membered heterocyclyl or 5-14 membered heteroaryl ring, and wherein each alkyl, alkenyl, alkynyl, carbocyclyl, heterocyclyl, aralkyl, aryl, and heteroaryl of R^{NP1} and R^{NP2} can be optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)— alkyl, C(O)— alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy, where “m” and “p” are independently 1, 2, 3, 4, 5 or 6.

Nitrogen protecting groups are well known in the art and include those described in detail in Greene's Protecting Groups in Organic Synthesis, P. G. M. Wuts, 5^{th} Edition, John Wiley & Sons, 2014, incorporated herein by reference.

Exemplary amide (e.g., —C(═O)R^{NP1}) nitrogen protecting groups include, but are not limited to, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxy acylamino)acetamide, 3-(p-hydroxylphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide, and o-(benzoyloxymethyl)benzamide.

Exemplary carbamate (e.g., —C(═O)OR^{NP1}) nitrogen protecting groups include, but are not limited to, methyl carbamate, ethyl carbamate, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC or Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxylpiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxylboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isobornyl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

Exemplary sulfonamide (e.g., —S(═O)₂R^{NP1}) nitrogen protecting groups include, but are not limited to, such as p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Additional exemplary nitrogen protecting groups include, but are not limited to, phenothiazinyl-(10)-acyl derivative, N′-p-toluenesulfonylaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuNP2inimide (Dts), N—2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxylphenyl)phenylmethyleneamine, N-cyclohexylideneamine, N-(5,5-dimethyl-3-oxo-1-cyclohexenyl)amine, N-borane and N-diphenylborinic acid derivative, N-[phenyl(pentNP1cylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys).

### Sulfur Protecting Groups

Some embodiments of the various aspects described herein include sulfur protecting group (also referred to as a thiol protecting group herein). Sulfur protecting groups include, but are not limited to, —R^{SP1}, —N(R^{SP2})₂, —C(═O)SR^{SP1}, —C(═O)R^{SP1}, —CO₂R^{SP1}, —C(═O)N(R^{SP2})₂, —C(═NR^{SP2})R^{SP1}, —C(═NR^{SP2})OR^{SP1}, —C(═NR^{SP2})N(R^{SP2})₂, —S(═O)R^{SP1}, —SO₂R^{SP1}, —Si(R^{SP1})₃, —P(R^{SP3})₂, —P(R^{SP3})+₃ X⁻, —P(OR^{SP3})₂, —P(OR^{SP3})⁺₃ X⁻, —P(═O)(R^{SP1})₂, —P(═O)(OR^{SP3})₂, and —P(═O)(N(R^{SP2})₂)₂, wherein

X⁻ is a counterion; each R^{SP1} is independently C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀alkenyl, heteroC₂₋₁₀alkynyl, C₃₋₁₀ carbocyclyl, 3-14 membered heterocyclyl, C₆₋₁₄ aryl, or 5-14 membered heteroaryl, or two R^{SP1} groups are joined to form a 3-14 membered heterocyclyl or 5-14 membered heteroaryl ring; each R^{SP2} is hydrogen, —OH, —OR^{SP1}, —N(R^{SP3})₂, —CN, —C(═O)R^{SP1}, C(═O)N(R^{SP3})₂, —CO₂R^{SP1}, —SO₂R^{SP1}, —C(═NR^{SP3})OR^{SP1}, —C(═NR^{SP3})N(R^{SP3})₂, —SO₂N(R^{SP3})₂, —SO₂R^{SP3}, —SO₂OR^{SP3}, —SOR^{SP1}, —C(═S)N(R^{SP3})₂, —C(═O)SR^{SP3}, —C(═S)SR^{SP3}, —P(═O)(R^{SP1})₂, —P(═O)(OR^{SP3})₂, —P(═O)(N(R^{SP3})₂)₂, C₁₋₁₀ alkyl, C₁₋₁₀ perhaloalkyl, C₂₋₁₀ alkenyl, C₂₋₁₀ alkynyl, heteroC₁₋₁₀alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{SP2} groups are joined to form a 3-14 membered heterocyclyl or 5-14 membered heteroaryl ring; and each R^{SP3} is independently hydrogen, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{SP3} groups are joined to form a 3-14 membered heterocyclyl or 5-14 membered heteroaryl ring; and wherein each alkyl, alkenyl, alkynyl, carbocyclyl, heterocyclyl, aralkyl, aryl, and heteroaryl of R^{SP1}, R^{SP2} and R^{SP3} can be optionally substituted with 1, 2, 3, 4 or 5 substituents independently selected from OH, CN, SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl (i.e., C₁-C₈alkoxy), O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)-alkyl, C(O)— alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy, where “m” and “p” are independently 1, 2, 3, 4, 5 or 6.

Sulfur protecting groups are well known in the art and include those described in detail in Greene's Protecting Groups in Organic Synthesis, P. G. M. Wuts, 5^{th} Edition, John Wiley & Sons, 2014, incorporated herein by reference.

It is noted that the nucleoside Formula IIIa-IIIg or IVa-IVg can be located anywhere in the oligonucleotide. In some embodiments, the nucleoside of Formula IIIa-IIIg or IVa-IVg is present at the 5′- or 3′-terminus of the oligonucleotide. In some embodiments, the nucleoside of Formula IIIa-IIIg or IVa-IVg is present at an internal position of the oligonucleotide.

In some embodiments of any one of the aspects described herein, the oligonucleotide further comprises, i.e., in addition to a nucleotide of Formula IIIa-IIIg or IVa-IVg, a nucleoside with a modified sugar. By a “modified sugar” is meant a sugar or moiety other than 2′-deoxy (i.e., 2′-H) or 2′-OH ribose sugar. Some exemplary nucleotides comprising a modified sugar are 2′-F ribose, 2′-OMe ribose, 2′-0,4′-C-methylene ribose (locked nucleic acid, LNA), anhydrohexitol (1,5-anhydrohexitol nucleic acid, HNA), cyclohexene (Cyclohexene nucleic acid, CeNA), 2′-methoxyethyl ribose, 2′-O-allyl ribose, 2′-C-allyl ribose, 2′-O—N-methylacetamido (2′-O-NMA) ribose, a 2′-O-dimethylaminoethoxyethyl (2′-O-DMAEOE) ribose, 2′-O-aminopropyl (2′-O-AP) ribose, 2′-F arabinose (2′-ara-F), threose (Threose nucleic acid, TNA), and 2,3-dihydroxylpropyl (glycol nucleic acid, GNA). It is noted that the nucleoside with the modified sugar can be present at any position of the oligonucleotide.

In some embodiments, the oligonucleotide further comprises at least one, e.g., 1, 2, 3, 4, 5, 6, 7, 8, 9, 10 or more 2′-fluoro (2′-F) nucleotides. For example, the oligonucleotide can comprise 1, 2, 3, 4, 5, 6, 7, 8, 9 or 10 2′-F nucleotides. It is noted that the 2′-F nucleotides can be present at any position of the oligonucleotide.

In some embodiments, the oligonucleotide comprises, e.g., solely comprises nucleosides of Formulae IIIa-IIIg and IVa-IVg and 2′-F nucleosides.

In some embodiments, the oligonucleotide further comprises at least one, e.g., 1, 2, 3, 4, 5, 6, 7, 8, 9, 10 or more 2′-OMe nucleotides. For example, the oligonucleotide can comprise 1, 2, 3, 4, 5, 6, 7, 8, 9 or 10 2′-OMe nucleotides. It is noted that the 2′-OMe nucleotides can be present at any position of the oligonucleotide.

In some embodiments, the oligonucleotide comprises, e.g., solely comprises nucleosides of Formulae IIIa-IIIg and IVa-IVg and 2′-OMe nucleosides. In some other embodiments, the oligonucleotide comprises, e.g., solely comprises solely comprises nucleosides of Formulae IIIa-IIIg and IVa-IVg, 2′-OMe nucleosides and 2′-F nucleosides.

In some embodiments, the oligonucleotide further comprises at least one, e.g., 1, 2, 3, 4, 5, 6, 7, 8, 9, 10 or more 2′-deoxy, e.g., 2′-H nucleotides. For example, the oligonucleotide can comprise 1, 2, 3, 4, 5, 6, 7, 8, 9 or 10 of 2′-deoxy, e.g., 2′-H nucleotides. It is noted that the 2′-deoxy, e.g., 2′-H nucleotides can be present at any position of the oligonucleotide. For example, the oligonucleotide can comprise a 2′-deoxy, e.g., 2′-H nucleotide at 1, 2, 3, 4, 5 or 6 of positions 2, 5, 7, 12, 14 and 16, counting from 5′-end of the oligonucleotide. In some embodiments, the oligonucleotide comprises a 2′-deoxy nucleotide at positions 5 and 7, counting from 5′-end of the oligonucleotide.

In some embodiments, the oligonucleotide comprises, e.g., solely comprises nucleosides of Formulae IIIa-IIIg and IVa-IVg and 2′-deoxy (2′-H) nucleotides. In some embodiments, the oligonucleotide comprises, e.g., solely comprises nucleosides of Formula IIIa-IIIg or IVa-IVg, 2′-OMe nucleosides, and 2′-deoxy (2′-H) nucleotides. In some embodiments, the oligonucleotide comprises, e.g., solely comprises nucleosides of Formulae IIIa-IIIg and IVa-IVg, 2′-F nucleosides and 2′-deoxy (2′-H) nucleotides. In some embodiments, the oligonucleotide comprises, e.g., solely comprises nucleosides of Formulae IIIa-IIIg and IVa-IVg, 2′-OMe nucleosides, 2′-F nucleosides and 2′-deoxy (2′-H) nucleotides.

In some embodiments of any one of the aspects described herein, the oligonucleotide further comprises, i.e., in addition to a nucleotiside of Formula IIIa-IIIg or IVa-IVg, a non-natural nucleobase. In some embodiments, the oligonucleotide can comprise one or more, e.g., 1, 2, 3, 4, 5, 6, 7, 8, 9, 10 or more nucleotides comprising an independently selected non-natural nucleobase. When present, a nucleotide comprising a non-natural nucleobase can be present anywhere in the oligonucleotide.

In some embodiments, the oligonucleotide further comprises a solid support linked thereto.

The oligonucleotides described herein can range from few nucleotides (e.g., 2, 3, 4, 5, 6, 7, 8, 9 or 10 nucleotides) in length to hundreds of nucleotides in length. For example, the oligonucleotide can be from 5 nucleotides to 100 nucleotides in length. In some embodiments, the oligonucleotide is from 10 nucleotides to 50 nucleotides in length. For example, the oligonucleotide is between 15 and 35, more generally between 18 and 25, yet more generally between 19 and 24, and most generally between 19 and 21 base pairs in length. In some embodiments, longer oligonucleotides of between 25 and 30 nucleotides in length are preferred. In some embodiments, shorter oligonucleotides of between 10 and 15 nucleotides in length are preferred. In another embodiment, the oligonucleotide is at least 21 nucleotides in length.

In some embodiments, the oligonucleotide described herein comprises a pattern of backbone chiral centers. In some embodiments, a common pattern of backbone chiral centers comprises at least 5 internucleotidic linkages in the Sp configuration. In some embodiments, a common pattern of backbone chiral centers comprises at least 6 internucleotidic linkages in the Sp configuration. In some embodiments, a common pattern of backbone chiral centers comprises at least 7 internucleotidic linkages in the Sp configuration. In some embodiments, a common pattern of backbone chiral centers comprises at least 8 internucleotidic linkages in the Sp configuration. In some embodiments, a common pattern of backbone chiral centers comprises at least 9 internucleotidic linkages in the Sp configuration. In some embodiments, a common pattern of backbone chiral centers comprises at least 10 internucleotidic linkages in the Sp configuration. In some embodiments, a common pattern of backbone chiral centers comprises at least 11 internucleotidic linkages in the Sp configuration. In some embodiments, a common pattern of backbone chiral centers comprises at least 12 internucleotidic linkages in the Sp configuration. In some embodiments, a common pattern of backbone chiral centers comprises at least 13 internucleotidic linkages in the Sp configuration. In some embodiments, a common pattern of backbone chiral centers comprises at least 14 internucleotidic linkages in the Sp configuration. In some embodiments, a common pattern of backbone chiral centers comprises at least 15 internucleotidic linkages in the Sp configuration. In some embodiments, a common pattern of backbone chiral centers comprises at least 16 internucleotidic linkages in the Sp configuration. In some embodiments, a common pattern of backbone chiral centers comprises at least 17 internucleotidic linkages in the Sp configuration. In some embodiments, a common pattern of backbone chiral centers comprises at least 18 internucleotidic linkages in the Sp configuration. In some embodiments, a common pattern of backbone chiral centers comprises at least 19 internucleotidic linkages in the Sp configuration. In some embodiments, a common pattern of backbone chiral centers comprises no more than 8 internucleotidic linkages in the Rp configuration. In some embodiments, a common pattern of backbone chiral centers comprises no more than 7 internucleotidic linkages in the Rp configuration. In some embodiments, a common pattern of backbone chiral centers comprises no more than 6 internucleotidic linkages in the Rp configuration. In some embodiments, a common pattern of backbone chiral centers comprises no more than 5 internucleotidic linkages in the Rp configuration. In some embodiments, a common pattern of backbone chiral centers comprises no more than 4 internucleotidic linkages in the Rp configuration. In some embodiments, a common pattern of backbone chiral centers comprises no more than 3 internucleotidic linkages in the Rp configuration. In some embodiments, a common pattern of backbone chiral centers comprises no more than 2 internucleotidic linkages in the Rp configuration. In some embodiments, a common pattern of backbone chiral centers comprises no more than 1 internucleotidic linkages in the Rp configuration. In some embodiments, a common pattern of backbone chiral centers comprises no more than 8 internucleotidic linkages which are not chiral (as a non-limiting example, a phosphodiester). In some embodiments, a common pattern of backbone chiral centers comprises no more than 7 internucleotidic linkages which are not chiral. In some embodiments, a common pattern of backbone chiral centers comprises no more than 6 internucleotidic linkages which are not chiral. In some embodiments, a common pattern of backbone chiral centers comprises no more than 5 internucleotidic linkages which are not chiral. In some embodiments, a common pattern of backbone chiral centers comprises no more than 4 internucleotidic linkages which are not chiral. In some embodiments, a common pattern of backbone chiral centers comprises no more than 3 internucleotidic linkages which are not chiral. In some embodiments, a common pattern of backbone chiral centers comprises no more than 2 internucleotidic linkages which are not chiral. In some embodiments, a common pattern of backbone chiral centers comprises no more than 1 internucleotidic linkages which are not chiral. In some embodiments, a common pattern of backbone chiral centers comprises at least 10 internucleotidic linkages in the Sp configuration, and no more than 8 internucleotidic linkages which are not chiral. In some embodiments, a common pattern of backbone chiral centers comprises at least 11 internucleotidic linkages in the Sp configuration, and no more than 7 internucleotidic linkages which are not chiral. In some embodiments, a common pattern of backbone chiral centers comprises at least 12 internucleotidic linkages in the Sp configuration, and no more than 6 internucleotidic linkages which are not chiral. In some embodiments, a common pattern of backbone chiral centers comprises at least 13 internucleotidic linkages in the Sp configuration, and no more than 6 internucleotidic linkages which are not chiral. In some embodiments, a common pattern of backbone chiral centers comprises at least 14 internucleotidic linkages in the Sp configuration, and no more than 5 internucleotidic linkages which are not chiral. In some embodiments, a common pattern of backbone chiral centers comprises at least 15 internucleotidic linkages in the Sp configuration, and no more than 4 internucleotidic linkages which are not chiral. In some embodiments, the internucleotidic linkages in the Sp configuration are optionally contiguous or not contiguous. In some embodiments, the internucleotidic linkages in the Rp configuration are optionally contiguous or not contiguous. In some embodiments, the internucleotidic linkages which are not chiral are optionally contiguous or not contiguous.

In some embodiments, the oligonucleotide described herein comprises a stereochemistry block. In some embodiments, a block is an Rp block in that each internucleotidic linkage of the block is Rp. In some embodiments, a 5′-block is an Rp block. In some embodiments, a 3′-block is an Rp block. In some embodiments, a block is an Sp block in that each internucleotidic linkage of the block is Sp. In some embodiments, a 5′-block is an Sp block. In some embodiments, a 3′-block is an Sp block. In some embodiments, provided oligonucleotides comprise both Rp and Sp blocks. In some embodiments, provided oligonucleotides comprise one or more Rp but no Sp blocks. In some embodiments, provided oligonucleotides comprise one or more Sp but no Rp blocks. In some embodiments, provided oligonucleotides comprise one or more PO blocks wherein each internucleotidic linkage in a natural phosphate linkage.

In some embodiments, the oligonculeotide described herein comprises a 5′-block is an Sp block wherein each sugar moiety comprises a 2′-fluoro modification. In some embodiments, a 5′-block is an Sp block wherein each of internucleotidic linkage is a modified internucleotidic linkage and each sugar moiety comprises a 2′-fluoro modification. In some embodiments, a 5′-block is an Sp block wherein each of internucleoside linkage is a phosphorothioate linkage and each sugar moiety comprises a 2′-fluoro modification. In some embodiments, a 5′-block comprises 4 or more nucleoside units. In some embodiments, a 5′-block comprises 5 or more nucleoside units. In some embodiments, a 5′-block comprises 6 or more nucleoside units. In some embodiments, a 5′-block comprises 7 or more nucleoside units. In some embodiments, a 3′-block is an Sp block wherein each sugar moiety comprises a 2′-fluoro modification. In some embodiments, a 3′-block is an Sp block wherein each of internucleotidic linkage is a modified internucleotidic linkage and each sugar moiety comprises a 2′-fluoro modification. In some embodiments, a 3′-block is an Sp block wherein each of internucleotidic linkage is a phosphorothioate linkage and each sugar moiety comprises a 2′-fluoro modification. In some embodiments, a 3′-block comprises 4 or more nucleoside units. In some embodiments, a 3′-block comprises 5 or more nucleoside units. In some embodiments, a 3′-block comprises 6 or more nucleoside units. In some embodiments, a 3′-block comprises 7 or more nucleoside units.

In some embodiments, oligonucleotide described herein comprises a type of nucleoside in a region or an oligonucleotide is followed by a specific type of internucleotidic linkage, e.g., natural phosphate linkage, modified internucleotidic linkage, Rp chiral internucleotidic linkage, Sp chiral internucleotidic linkage, etc. In some embodiments, A is followed by Sp. In some embodiments, A is followed by Rp. In some embodiments, A is followed by natural phosphate linkage (PO). In some embodiments, U is followed by Sp. In some embodiments, U is followed by Rp. In some embodiments, U is followed by natural phosphate linkage (PO). In some embodiments, C is followed by Sp. In some embodiments, C is followed by Rp. In some embodiments, C is followed by natural phosphate linkage (PO). In some embodiments, G is followed by Sp. In some embodiments, G is followed by Rp. In some embodiments, G is followed by natural phosphate linkage (PO). In some embodiments, C and U are followed by Sp. In some embodiments, C and U are followed by Rp. In some embodiments, C and U are followed by natural phosphate linkage (PO). In some embodiments, A and G are followed by Sp. In some embodiments, A and G are followed by Rp.

In some embodiments of any one of the aspects described herein, the oligonucleotides described herein are 5′ phosphorylated or include a phosphoryl analog at the 5′ prime terminus. 5′-phosphate modifications include those which are compatible with RISC mediated gene silencing. Suitable modifications include: 5′-monophosphate ((HO)₂(O)P—O-5′); 5′-diphosphate ((HO)₂(O)P—O—P(HO)(O)—O-5′); 5′-triphosphate ((HO)₂(0)P—O—(HO)(O)P—O—P(HO)(O)—O-5′); 5′-guanosine cap (7-methylated or non-methylated) (7m-G-O-5′-(HO)(O)P—O—(HO)(O)P—O—P(HO)(O)—O-5′); 5′-adenosine cap (Appp), and any modified or unmodified nucleotide cap structure (N—O-5′-(HO)(O)P—O—(HO)(O)P—O—P(HO)(O)—O-5′); 5′-monothiophosphate (phosphorothioate; (HO)₂(S)P-0-5′); 5′-monodithiophosphate (phosphorodithioate; (HO)(HS)(S)P—O-5′), 5′-phosphorothiolate ((HO)2(O)P—S-5′); any additional combination of oxygen/sulfur replaced monophosphate, diphosphate and triphosphates (e.g. 5′-alpha-thiotriphosphate, 5′-gamma-thiotriphosphate, etc.), 5′-phosphoramidates ((HO)₂(O)P—NH-5′, (HO)(NH₂)(O)P—O-5′), 5′-alkylphosphonates (e.g., RP(OH)(O)—O-5′-, R=alkyl, e.g., methyl, ethyl, isopropyl, propyl, etc.), 5′-alkenylphosphonates (i.e. vinyl, substituted vinyl, e.g., OH)₂(0)P-5′-CH═ or (OH)₂(0)P-5′-CH₂—), 5′-alkyletherphosphonates (e.g., R(OH)(O)P—O-5′, R=alkylether, e.g., methoxymethyl (MeOCH2-), ethoxymethyl, etc.) Other exemplary 5′-modifications include where Z is optionally substituted alkyl at least once, e.g., ((HO)₂(X)P—O[—(CH₂)ₐ—O—P(X)(OH)—O]_{b}-5′, ((HO)₂(X)P—O[—(CH₂)ₐ—P(X)(OH)—O]_{b}-5′, ((HO)₂(X)P—[—(CH₂)ₐ—O—P(X)(OH)—O]_{b}-5′; dialkyl terminal phosphates and phosphate mimics: HO[—(CH₂)ₐ—O—P(X)(OH)—O]_{b}-5′, H2N[—(CH₂)ₐ—O—P(X)(OH)—O]_{b}-5′, H[—(CH₂)ₐ—O—P(X)(OH)—O]_{b}-5′, Me₂N[—(CH₂)ₐ—O—P(X)(OH)—O]_{b}-5′, HO[—(CH₂)ₐ—P(X)(OH)—O]_{b}-5′, H₂N[—(CH₂)ₐ—P(X)(OH)—O]_{b}-5′, H[—(CH₂)ₐ—P(X)(OH)—O]_{b}-5′, Me₂N[—(CH₂)ₐ—P(X)(OH)—O]_{b}-5′, wherein a and b are each independently 1-10. Other embodiments, include replacement of oxygen and/or sulfur with BH₃, BH₃⁻ and/or Se.

In some embodiments of any one of the aspects described herein, the oligonucleotide comprises a 5′-vinylphosphonate group. For example, the oligonucleotide comprises a 5′-E-vinyl phosphonate group. In some other non-limiting example, the oligonucleotide comprises a 5′-Z-vinylphosphonate group.

In some embodiments of any one of the aspects, the oligonucleotide described herein comprises a 5′-morpholino, a 5′-dimethylamino, a 5′-deoxy, an inverted abasic, or an inverted abasic locked nucleic acid modification at the 5′-end.

In some embodiments of any one of the aspects, the oligonucleotide described herein can comprise a thermally destabilizing modification. For example, the oligonucleotide can comprise at least one thermally destabilizing modification of the duplex within the first 9 nucleotide positions, counting from the 5′-end of the oligonucleotide. In some embodiments, the thermally destabilizing modification is located at position 2, 3, 4, 5, 6, 7, 8 or 9, counting from the 5′-end of the antisense strand. In some embodiments, thermally destabilizing modification is located in positions 2-9, or preferably positions 4-8, counting from the 5′-end of the oligonucleotide. In some further embodiments, the thermally destabilizing modification is located at position 5, 6, 7 or 8, counting from the 5′-end of the oligonucleotide. In still some further embodiments, the thermally destabilizing modification is located at position 7, counting from the 5′-end of the oligonucleotide.

The term “thermally destabilizing modification(s)” includes modification(s) that would result with a dsRNA with a lower overall melting temperature (Tm) (preferably a Tm with one, two, three or four degrees lower than the Tm of the dsRNA without having such modification(s). In some embodiments, the thermally destabilizing modification is located at position 2, 3, 4, 5, 6, 7, 8 or 9, counting from the 5′-end of the antisense strand.

The thermally destabilizing modifications can include, but are not limited to, abasic modification; mismatch with the opposing nucleotide in the opposing strand; and sugar modification such as 2′-deoxy modification or acyclic nucleotide, e.g., unlocked nucleic acids (UNA) or glycol nucleic acid (GNA). For example, the thermally destabilizing modifications can include, but are not limited to, mUNA and GNA building blocks as follows:

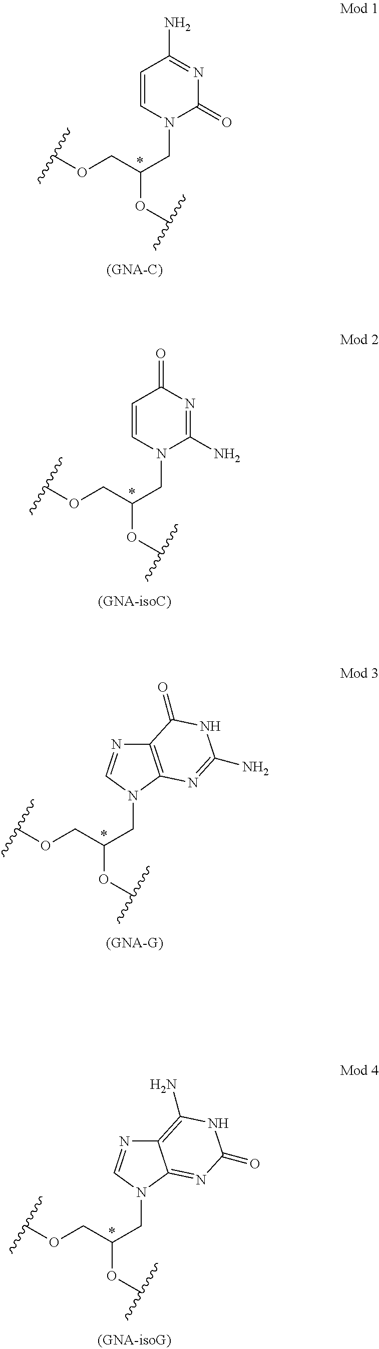 [SMILES: *OCC(Cn1ccc(N)nc1=O)O* ; *OCC(Cn1ccc(=O)nc1N)O* ; *OCC(Cn1cnc2c(=O)[nH]c(N)nc21)O* ; *OCC(Cn1cnc2c(N)[nH]c(=O)nc21)O*]

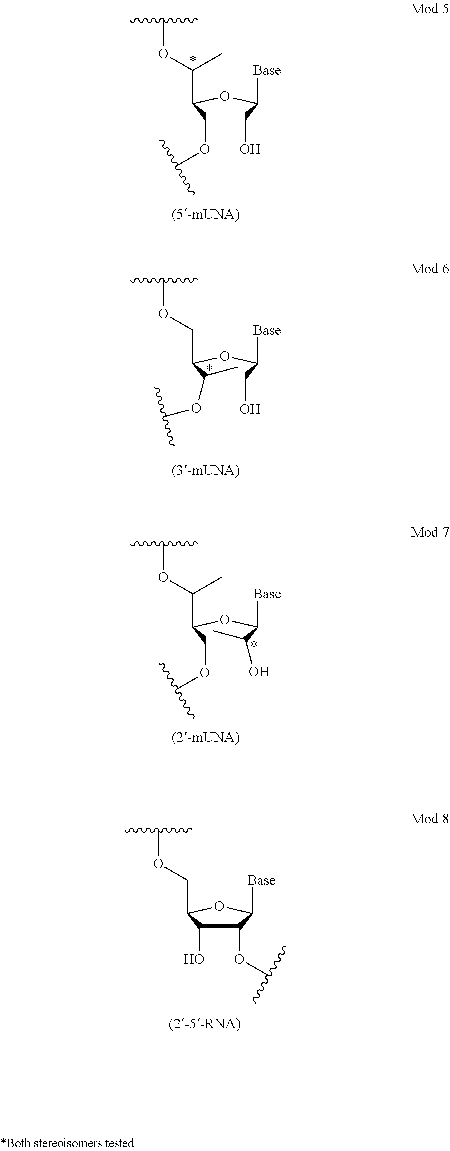

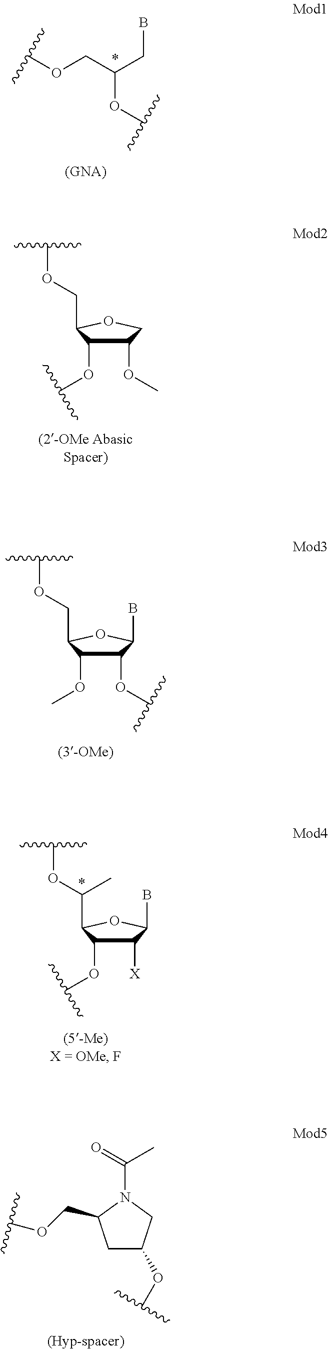

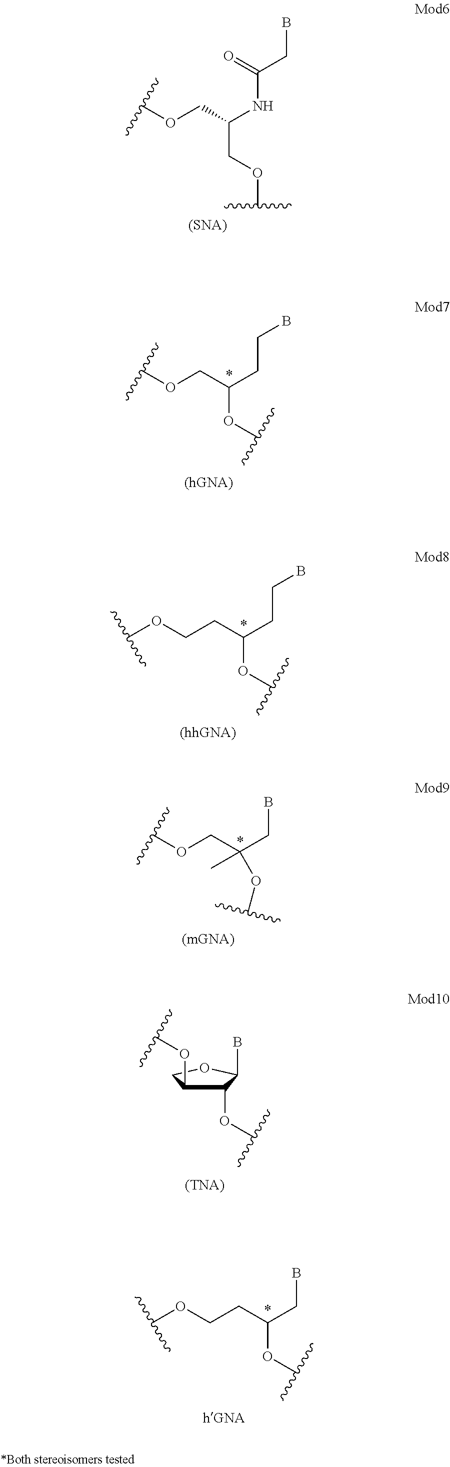

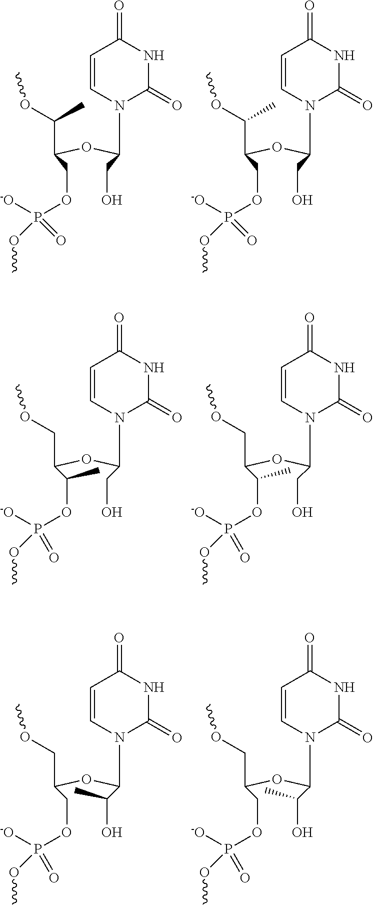

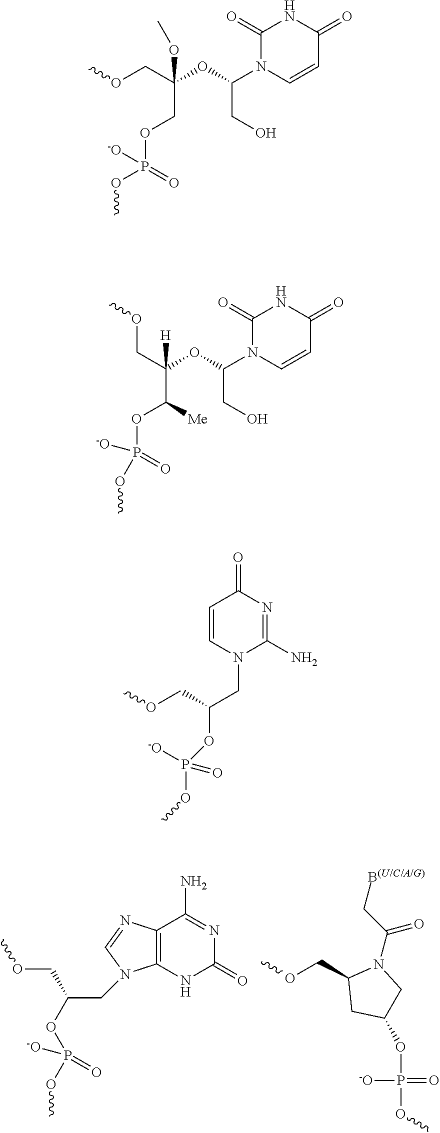

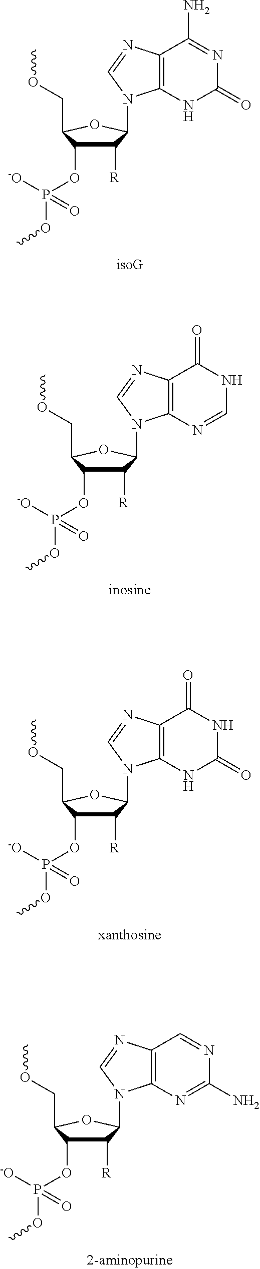

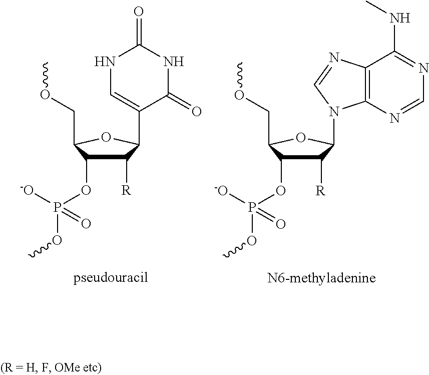

In some embodiments, the destabilizing modification is selected from the group consisting of GNA-isoC, GNA-isoG, 5′-mUNA, 4′-mUNA, 3′-mUNA, and 2′-mUNA.

In some embodiments, the destabilizing modification mUNA is selected from the group consisting of

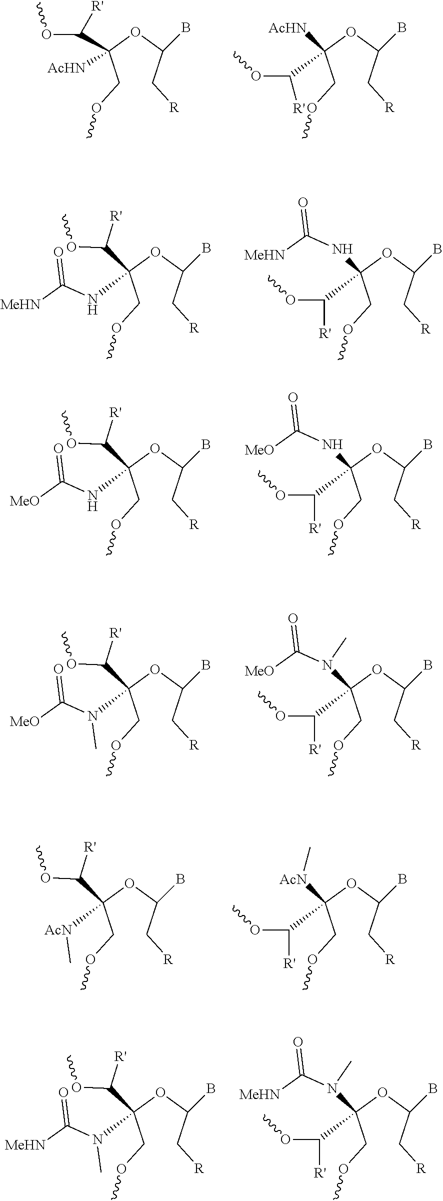

R=H, OH; OMe; C₁, F; OH; O—(CH₂)₂OMe; SMe, NMe₂; NH₂; Me; CCH (alkyne), O-nPr; O-alkyl; O-alkylamino;
R′=H, Me;
B=A; C; 5-Me-C; G; I; U; T; Y; 2-thiouridine; 4-thiouridine; C5-modified pyrimidines; C2-modified purines; N8-modified purines; phenoxazine; G-clamp; non-canonical mono, bi and tricyclic heterocycles; pseudouracil; isoC; isoG; 2,6-diaminopurine; pseudocytosine; 2-aminopurine; xanthosine; N6-alkyl-A; 06-alkyl-G; 2-thiouridine; 4-thiouridine; C5-modified pyrimidines; C₂-modified purines; N8-modified purines; 7-deazapurines, phenoxazine; G-clamp; non-canonical mono, bi and tricyclic heterocycles; and
Stereochemistry is R or S and combination of R and S for the unspecified chiral centers.

In some embodiments, the destabilizing modification mUNA is selected from the group consisting of

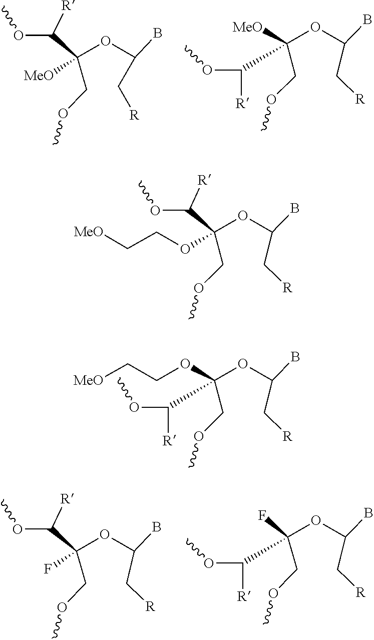

R=H, OH; OMe; C₁, F; OH; O—(CH₂)₂OMe; SMe, NMe₂; NH₂; Me; CCH (alkyne), O-nPr; O-alkyl; O-alkylamino;
R′=H, Me;
B=A; C; 5-Me-C; G; I; U; T; Y; 2-thiouridine; 4-thiouridine; C5-modified pyrimidines; C2-modified purines; N8-modified purines; phenoxazine; G-clamp; non-canonical mono, bi and tricyclic heterocycles; pseudouracil; isoC; isoG; 2,6-diaminopurine; pseudocytosine; 2-aminopurine; xanthosine; N6-alkyl-A; 06-alkyl-G; 2-thiouridine; 4-thiouridine; C5-modified pyrimidines; C₂-modified purines; N8-modified purines; 7-deazapurines, phenoxazine; G-clamp; non-canonical mono, bi and tricyclic heterocycles; and
Stereochemistry is R or S and combination of R and S for the unspecified chiral centers.

In some embodiments, the destabilizing modification mUNA is selected from the group consisting of

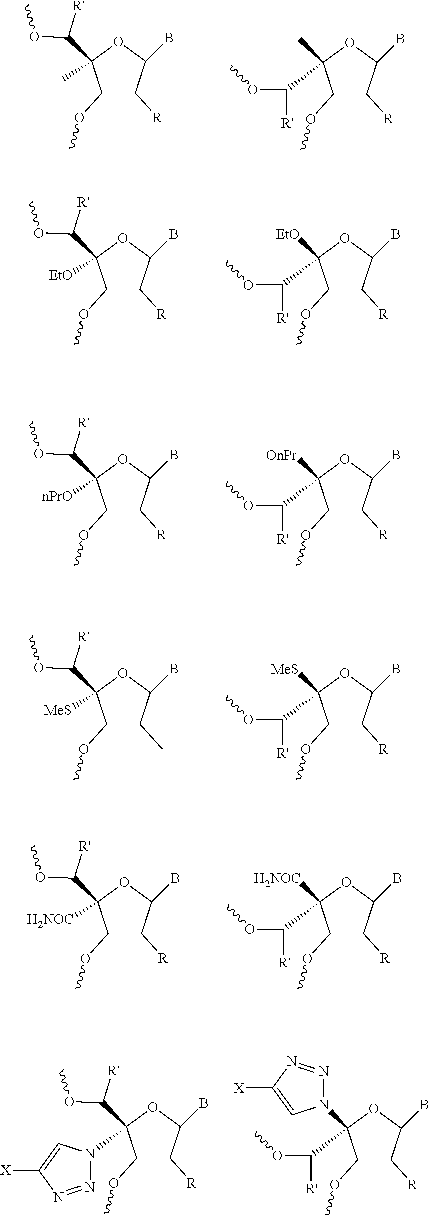

R=H, OMe; F; OH; O—(CH₂)₂OMe; SMe, NMe₂; NH₂; Me; O-nPr; O-alkyl; O-alkylamino;
R′=H, Me;
B=A; C; 5-Me-C; G; I; U; T; Y; 2-thiouridine; 4-thiouridine; C5-modified pyrimidines; C2-modified purines; N8-modified purines; phenoxazine; G-clamp; non-canonical mono, bi and tricyclic heterocycles; pseudouracil; isoC; isoG; 2,6-diaminopurine; pseudocytosine; 2-aminopurine; xanthosine; N6-alkyl-A; 06-alkyl-G; 7-deazapurines; and
Stereochemistry is R or S and combination of R and S for the unspecified chiral centers.

In some embodiments, the destabilizing modification mUNA is selected from the group consisting of

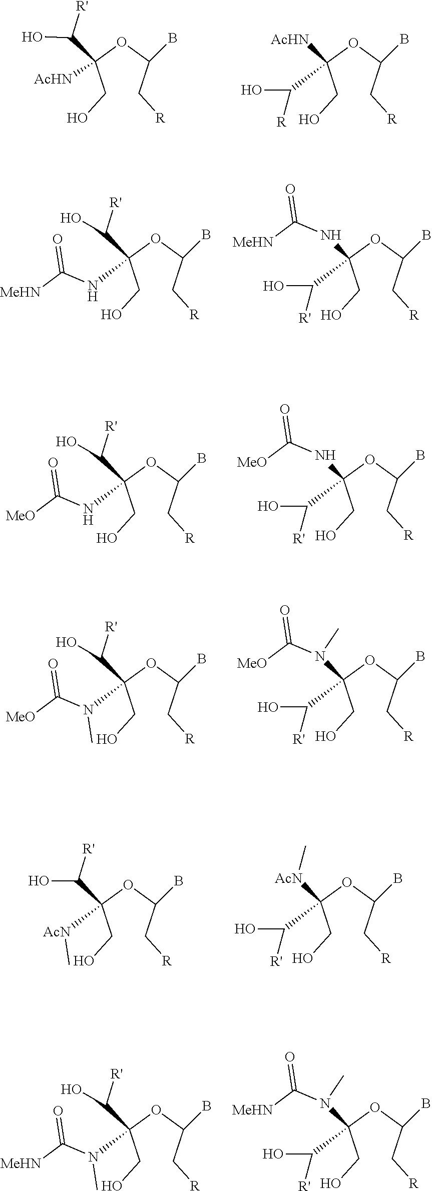

R=H, OH; OMe; C₁, F; OH; O—(CH₂)₂OMe; SMe, NMe₂; NH₂; Me; CCH (alkyne), O-nPr; O-alkyl; O-alkylamino;
R′=H, Me;
B=A; C; 5-Me-C; G; I; U; T; Y; 2-thiouridine; 4-thiouridine; C5-modified pyrimidines; C2-modified purines; N8-modified purines; phenoxazine; G-clamp; non-canonical mono, bi and tricyclic heterocycles; pseudouracil; isoC; isoG; 2,6-diaminopurine; pseudocytosine; 2-aminopurine; xanthosine; N6-alkyl-A; 06-alkyl-G; 2-thiouridine; 4-thiouridine; C5-modified pyrimidines; C₂-modified purines; N8-modified purines; 7-deazapurines, phenoxazine; G-clamp; non-canonical mono, bi and tricyclic heterocycles; and
Stereochemistry is R or S and combination of R and S for the unspecified chiral centers

In some embodiments, the destabilizing modification mUNA is selected from the group consisting of

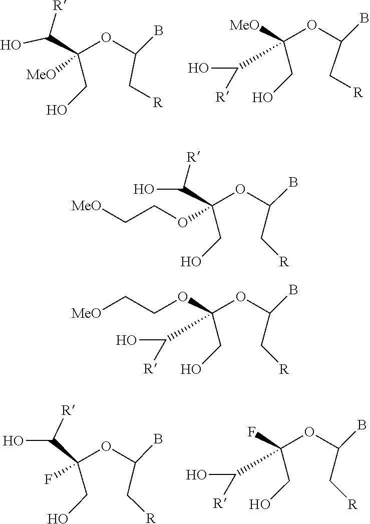

R=H, OH; OMe; C₁, F; OH; O—(CH₂)₂OMe; SMe, NMe₂; NH₂; Me; CCH (alkyne), O-nPr; O-alkyl; O-alkylamino;
R′=H, Me;
B=A; C; 5-Me-C; G; I; U; T; Y; 2-thiouridine; 4-thiouridine; C5-modified pyrimidines; C2-modified purines; N8-modified purines; phenoxazine; G-clamp; non-canonical mono, bi and tricyclic heterocycles; pseudouracil; isoC; isoG; 2,6-diaminopurine; pseudocytosine; 2-aminopurine; xanthosine; N6-alkyl-A; 06-alkyl-G; 2-thiouridine; 4-thiouridine; C5-modified pyrimidines; C₂-modified purines; N8-modified purines; 7-deazapurines, phenoxazine; G-clamp; non-canonical mono, bi and tricyclic heterocycles; and
Stereochemistry is R or S and combination of R and S for the unspecified chiral centers

In some embodiments, the modification mUNA is selected from the group consisting of

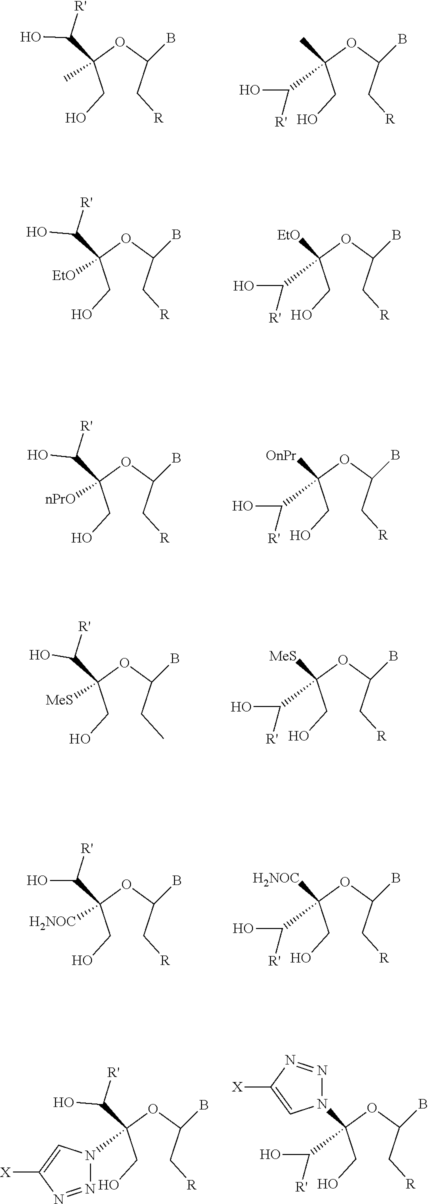

R=H, OMe; F; OH; O—(CH₂)₂OMe; SMe, NMe₂; NH₂; Me; O-nPr; O-alkyl; O-alkylamino;
R′=H, Me;
B=A; C; 5-Me-C; G; I; U; T; Y; 2-thiouridine; 4-thiouridine; C5-modified pyrimidines; C2-modified purines; N8-modified purines; phenoxazine; G-clamp; non-canonical mono, bi and tricyclic heterocycles; pseudouracil; isoC; isoG; 2,6-diaminopurine; pseudocytosine; 2-aminopurine; xanthosine; N6-alkyl-A; 06-alkyl-G; 7-deazapurines; and
Stereochemistry is R or S and combination of R and S for the unspecified chiral centers

Exemplary abasic modifications include, but are not limited to the following:

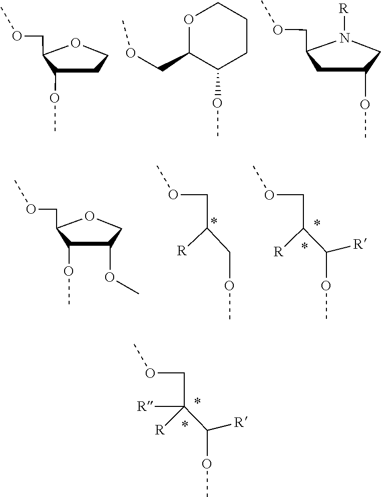

Wherein R=H, Me, Et or OMe; R′=H, Me, Et or OMe; R″=H, Me, Et or OMe

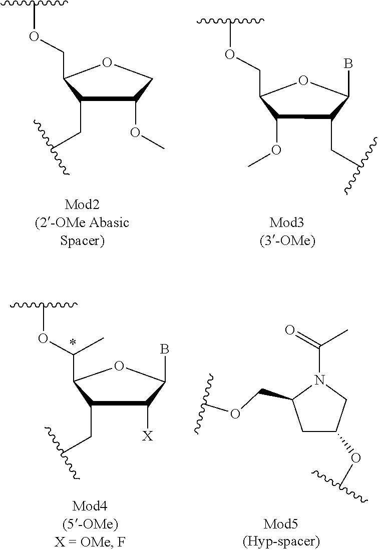

wherein B is a modified or unmodified nucleobase and the asterisk on each structure represents either R, S or racemic.

Exemplified sugar modifications include, but are not limited to the following:

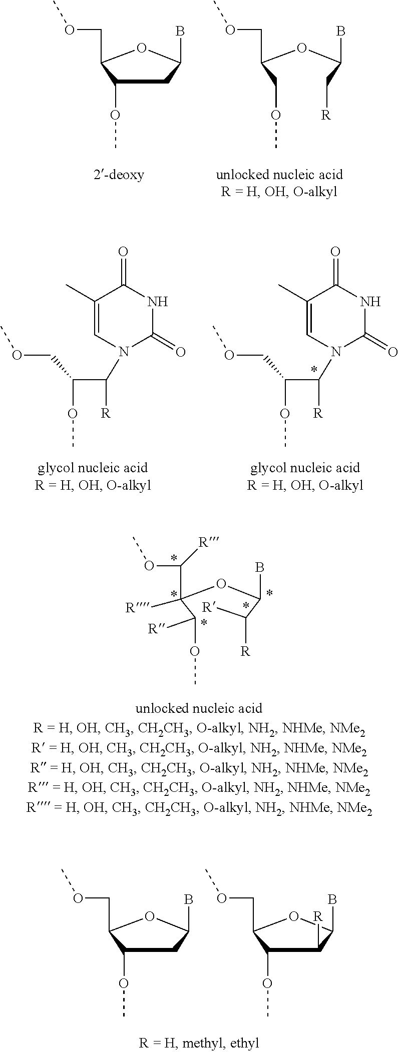

wherein B is a modified or unmodified nucleobase and the asterisk on each structure represents either R, S or racemic.

In some embodiments the thermally destabilizing modification of the duplex is selected from the mUNA and GNA building blocks described in Examples 1-3 herein. In some embodiments, the destabilizing modification is selected from the group consisting of GNA-isoC, GNA-isoG, 5′-mUNA, 4′-mUNA, 3′-mUNA, and 2′-mUNA. In some further embodiments of this, the dsRNA molecule further comprises at least one thermally destabilizing modification selected from the group consisting of GNA, 2′-OMe, 3′-OMe, 5′-Me, Hy p-spacer, SNA, hGNA, hhGNA, mGNA, TNA and h′GNA (Mod A-Mod K).

The term “acyclic nucleotide” refers to any nucleotide having an acyclic ribose sugar, for example, where any of bonds between the ribose carbons (e.g., C1′-C2′, C2′-C3′, C3′-C4′, C4′-O4′, or C1′-O4′) is absent and/or at least one of ribose carbons or oxygen (e.g., C1′, C2′, C3′, C4′ or O4′) are independently or in combination absent from the nucleotide. In some embodiments, acyclic nucleotide is

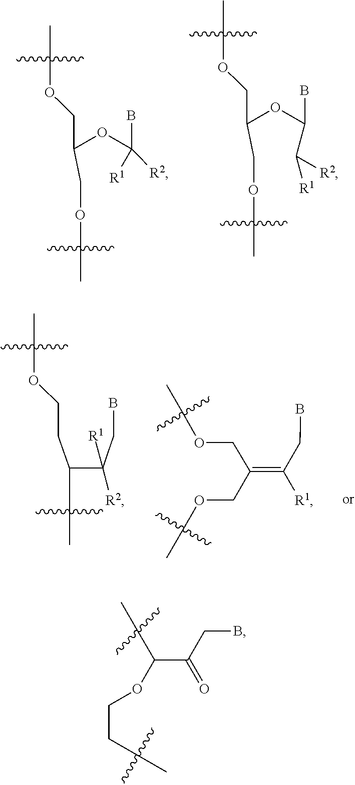

wherein B is a modified or unmodified nucleobase, R¹ and R² independently are H, halogen, OR3, or alkyl; and R³ is H, alkyl, cycloalkyl, aryl, aralkyl, heteroaryl or sugar). The term “UNA” refers to unlocked acyclic nucleic acid, wherein any of the bonds of the sugar has been removed, forming an unlocked “sugar” residue. In one example, UNA also encompasses monomers with bonds between C1′-C4′ being removed (i.e. the covalent carbon-oxygen-carbon bond between the C1′ and C4′ carbons). In another example, the C2′-C3′ bond (i.e. the covalent carbon-carbon bond between the C2′ and C3′ carbons) of the sugar is removed (see Mikhailov et. al., Tetrahedron Letters, 26 (17): 2059 (1985); and Fluiter et al., Mol. Biosyst., 10: 1039 (2009), which are hereby incorporated by reference in their entirety). The acyclic derivative provides greater backbone flexibility without affecting the Watson-Crick pairings. The acyclic nucleotide can be linked via 2′-5′ or 3′-5′ linkage.

The term ‘GNA’ refers to glycol nucleic acid which is a polymer similar to DNA or RNA but differing in the composition of its “backbone” in that is composed of repeating glycerol units linked by phosphodiester bonds:

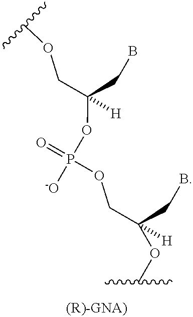

The thermally destabilizing modification of the duplex can be mismatches (i.e., noncomplementary base pairs) between the thermally destabilizing nucleotide and the opposing nucleotide in the opposite strand within the dsRNA duplex. Exemplary mismatch base pairs include G:G, G:A, G:U, G:T, A:A, A:C, C:C, C:U, C:T, U:U, T:T, U:T, or a combination thereof. Other mismatch base pairings known in the art are also amenable to the present invention. A mismatch can occur between nucleotides that are either naturally occurring nucleotides or modified nucleotides, i.e., the mismatch base pairing can occur between the nucleobases from respective nucleotides independent of the modifications on the ribose sugars of the nucleotides. In certain embodiments, the dsRNA molecule contains at least one nucleobase in the mismatch pairing that is a 2′-deoxy nucleobase; e.g., the 2′-deoxy nucleobase is in the sense strand.

In some embodiments, the thermally destabilizing modification of the duplex in the seed region of the antisense strand includes nucleotides with impaired W—C H-bonding to complementary base on the target mRNA, such as:

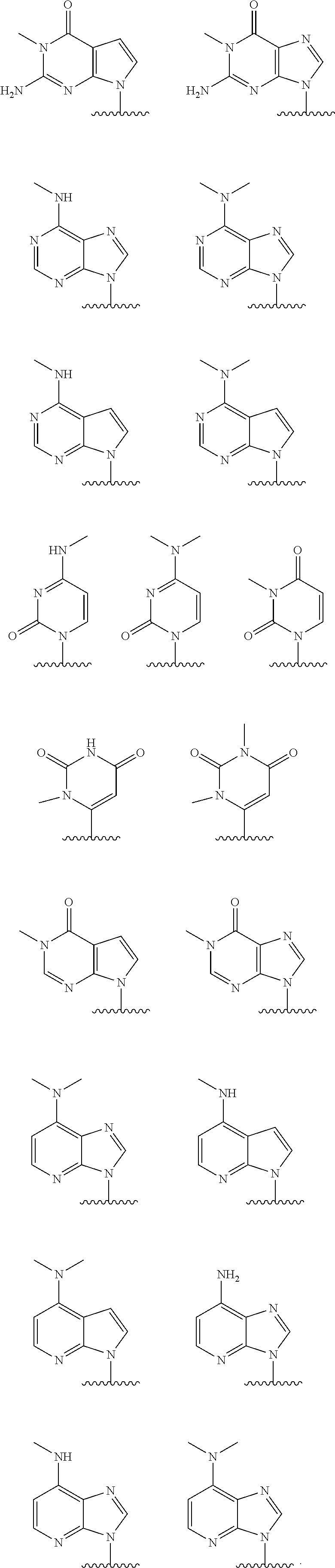

More examples of abasic nucleotide, acyclic nucleotide modifications (including UNA and GNA), and mismatch modifications have been described in detail in WO 2011/133876, which is herein incorporated by reference in its entirety.

The thermally destabilizing modifications may also include universal base with reduced or abolished capability to form hydrogen bonds with the opposing bases, and phosphate modifications.

In some embodiments, the thermally destabilizing modification includes nucleotides with non-canonical bases such as, but not limited to, nucleobase modifications with impaired or completely abolished capability to form hydrogen bonds with bases in the opposite strand. These nucleobase modifications have been evaluated for destabilization of the central region of the dsRNA duplex as described in WO 2010/0011895, which is herein incorporated by reference in its entirety. Exemplary nucleobase modifications are:

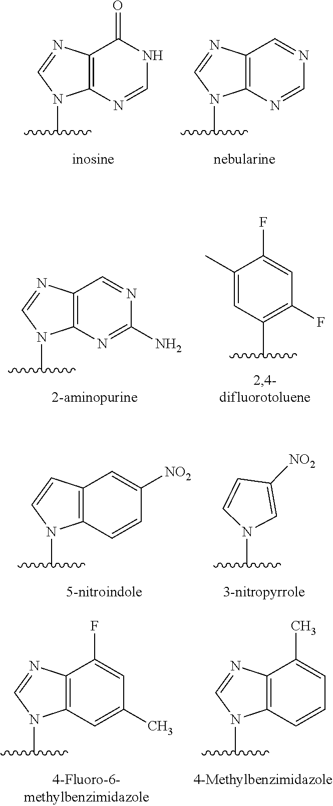

In some embodiments, the thermally destabilizing modification of the duplex in the seed region of the antisense strand includes one or more α-nucleotide complementary to the base on the target mRNA, such as:

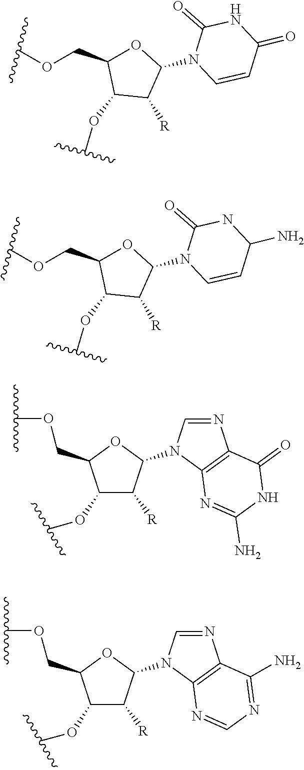

wherein R is H, OH, OCH₃, F, NH₂, NHMe, NMe₂ or O-alkyl

Exemplary phosphate modifications known to decrease the thermal stability of dsRNA duplexes compared to natural phosphodiester linkages are:

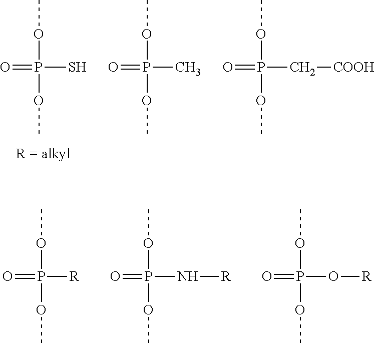

The alkyl for the R group can be a C₁-C₆alkyl. Specific alkyls for the R group include, but are not limited to methyl, ethyl, propyl, isopropyl, butyl, pentyl and hexyl.

In some embodiments of any one of the aspects described herein, the oligonucleotide can comprise one or more stabilizing modifications. For example, the oligonucleotide can comprise at least two (e.g., two, three, four, five, six, seven, eight, nine, ten or more) stabilizing modifications.

In some embodiments, the oligonucleotide comprises at least two (e.g., two, three, four, five, six, seven, eight, nine, ten or more) stabilizing modifications. Without limitations, a stabilizing modification in the oligonucleotide can be present at any positions. In some embodiments, the oligonucleotide comprises stabilizing modifications at positions 2, 6, 8, 9, 14 and 16, counting from the 5′-end. In some other embodiments, the oligonucleotide comprises stabilizing modifications at positions 2, 6, 14 and 16, counting from the 5′-end. In still some other embodiments, the oligonucleotide comprises stabilizing modifications at positions 2, 14 and 16, counting from the 5′-end. In some embodiments, the oligonucleotide comprises stabilizing modifications at positions 7, 10 and 11, counting from the 5′-end. In some other embodiments, the oligonucleotide comprises stabilizing modifications at positions 7, 9, 10 and 11, counting from the 5′-end.

In some embodiments, the oligonucleotide comprises at least one stabilizing modification adjacent to a destabilizing modification. For example, the stabilizing modification can be the nucleotide at the 5′-end or the 3′-end of the destabilizing modification, i.e., at position -1 or +1 from the position of the destabilizing modification. In some embodiments, the oligonucleotide comprises a stabilizing modification at each of the 5′-end and the 3′-end of the destabilizing modification, i.e., positions -1 and +1 from the position of the destabilizing modification.

In some embodiments, the oligonucleotide comprises at least two stabilizing modifications at the 3′-end of a destabilizing modification, i.e., at positions +1 and +2 from the position of the destabilizing modification.

Exemplary thermally stabilizing modifications include, but are not limited to 2′-fluoro modifications. Other thermally stabilizing modifications include, but are not limited to LNA.

### Double-Stranded RNAs

The skilled person is well aware that double-stranded RNAs comprising a duplex structure of between 20 and 23, but specifically 21, base pairs have been hailed as particularly effective in inducing RNA interference (Elbashir et al., EMBO 2001, 20:6877-6888). However, others have found that shorter or longer double-stranded oligonucleotides can be effective as well.

Accordingly, in one aspect, provided herein is a double-stranded RNA (dsRNA) comprising a first strand (also referred to as an antisense strand or a guide strand) and a second strand (also referred to as a sense strand or passenger strand, wherein at least one of the first (i.e., the antisense strand) or the second strand (i.e., the sense strand) is an oligonucleotide described herein. In other words, at least one of the first (i.e., the antisense strand) or the second strand (i.e., the sense strand) comprises at least one nucleotide of Formula IIIa-IIIg or IVa-IVg.

In some embodiments of any one of the aspects described herein, the sense strand is an oligonucleotide described herein. In other words, the sense strand comprises at least one nucleotide of Formula IIIa-IIIg or IVa-IVg.

In some embodiments of any one of the aspects described herein, the antisense strand is an oligonucleotide described herein. In other words, the antisense strand comprises at least one nucleotide of Formula IIIa-IIIg or IVa-IVg.

In some embodiments of the various aspects described herein, the antisense strand is substantially complementary to a target nucleic acid, e.g., a target gene or mRNA gene and the dsRNA is capable of inducing targeted cleavage of the target nucleic acid.

Each strand of the dsRNA molecule can range from 15-35 nucleotides in length. For example, each strand can be between, 17-35 nucleotides in length, 17-30 nucleotides in length, 25-35 nucleotides in length, 27-30 nucleotides in length, 17-23 nucleotides in length, 17-21 nucleotides in length, 17-19 nucleotides in length, 19-25 nucleotides in length, 19-23 nucleotides in length, 19-21 nucleotides in length, 21-25 nucleotides in length, or 21-23 nucleotides in length. Without limitations, the sense and antisense strands can be equal length or unequal length. For example, the sense strand and the antisense strand independently have a length of 18, 19, 20, 21, 22, 23, 24 or 25 nucleotides.

In some embodiments, the antisense strand is of length 15-35 nucleotides. In some embodiments, the antisense strand is 15-35, 17-35, 17-30, 25-35, 27-30, 17-23, 17-21, 17-19, 19-25, 19-23, 19-21, 21-25, 21-25, or 21-23 nucleotides in length. For example, the antisense strand can be 15, 16, 17, 18, 19, 20, 21, 22, 23, 24, 25, 26, 27, 28, 29, 30, 31, 32, 33, 34 or 35 nucleotides in length. In some embodiments, the antisense strand is 19, 20, 21, 22, 23, 24 or 25 nucleotides in length. For example, the antisense strand is 21, 22, 23, 24 or 25 nucleotides in length. In some particular embodiments, the antisense strand is 22, 23 or 24 nucleotides in length. For example, the antisense strand is 23 nucleotides in length.

Similar to the antisense strand, the sense strand can be, in some embodiments, 15-35 nucleotides in length. In some embodiments, the sense strand is 15-35, 17-35, 17-30, 25-35, 27-30, 17-23, 17-21, 17-19, 19-25, 19-23, 19-21, 21-25, 21-25, or 21-23 nucleotides in length. For example, the sense strand can be 15, 16, 17, 18, 19, 20, 21, 22, 23, 24, 25, 26, 27, 28, 29, 30, 31, 32, 33, 34 or 35 nucleotides in length. In some embodiments, the sense strand is 17, 18, 19, 20, 21, 22, 23, 24 or 25 nucleotides in length. For example, the sense strand is 19, 20, 21, 22 or 23 nucleotides in length. In some particular embodiments, the sense strand is 20, 21 or 22 nucleotides in length. For example, the sense strand is 21nucleotides in length

In some embodiments, the sense strand can be 15-35 nucleotides in length, and the antisense strand can be independent from the sense strand, 15-35 nucleotides in length. In some embodiments, the sense strand is 15-35, 17-35, 17-30, 25-35, 27-30, 17-23, 17-21, 17-19, 19-25, 19-23, 19-21, 21-25, 21-25, or 21-23 nucleotides in length, and the antisense strand is independently 15-35, 17-35, 17-30, 25-35, 27-30, 17-23, 17-21, 17-19, 19-25, 19-23, 19-21, 21-25, 21-25, or 21-23 nucleotides in length. For example, the sense and the antisense strand can be independently 15, 16, 17, 18, 19, 20, 21, 22, 23, 24, 25, 26, 27, 28, 29, 30, 31, 32, 33, 34 or 35 nucleotides in length. In some embodiments, the sense strand and the antisense strand are independently 17, 18, 19, 20, 21, 22, 23, 24 or 25 nucleotides in length. For example, the sense strand is 19, 20, 21, 22 or 23 nucleotides in length and the antisense strand is 21, 22, 23, 24 or 25 nucleotides in length. In some particular embodiments, the sense strand is 20, 21 or 22 nucleotides in length and the antisense strand is 22, 23 or 24 nucleotides in length. For example, the sense strand is 21 nucleotides in length and the antisense strand is 23 nucleotides in length.

The sense strand and antisense strand typically form a double-stranded or duplex region. Without limitations, the duplex region of a dsRNA agent described herein can be 12-35 nucleotide (or base) pairs in length. For example, the duplex region can be between 14-35 nucleotide pairs in length, 17-30 nucleotide pairs in length, 25-35 nucleotides in length, 27-35 nucleotide pairs in length, 17-23 nucleotide pairs in length, 17-21 nucleotide pairs in length, 17-19 nucleotide pairs in length, 19-25 nucleotide pairs in length, 19-23 nucleotide pairs in length, 19-21 nucleotide pairs in length, 21-25 nucleotide pairs in length, or 21-23 nucleotide pairs in length. In another example, the duplex region is selected from 15, 16, 17, 18, 19, 20, 21, 22, 23, 24, 25, 26, and 27 nucleotide pairs in length. In some embodiments, the duplex region is 18, 19, 20, 21, 22, 23, 24 or 25 nucleotide pairs in length. For example, the duplex region is 19, 20, 21, 22 or 23 nucleotide pairs in length. In some embodiments, the duplex region is 20, 21 or 22 nucleotide pairs in length. For example, the dsRNA molecule has a duplex region of 21 base pairs.

As described herein, the dsRNA molecule described herein can comprise at least one, e.g., 1, 2, 3, 4, 5, 6, 7, 8, 9, 10 or more of nucleotide of Formulae IIIa-IIIg and IVa-IVg. Without limitations, the nucleotides of Formulae IIIa-IIIg and IVa-IVg all can be present in one strand. The nucleotide of Formulae IIIa-IIIg and IVa-IVg may occur on any nucleotide of the sense strand or antisense strand or both in any position of the strand.

In some embodiments, the sense strand comprises 1, 2, 3, 4, 5, 6, 7, 8, 9, 10 or more nucleotides of Formula IIIa-IIIg or IVa-IVg described herein. The nucleotide of Formula IIIa-IIIg or IVa-IVg described herein can be present at any position of the sense strand. For example, the nucleotide of Formula IIIa-IIIg or IVa-IVg described herein can be present at a terminal region of the sense strand. For example, the nucleotide of Formula IIIa-IIIg or IVa-IVg described herein can be present at one or more of positions 1, 2, 3 and 4, counting from the 5′-end of the sense strand. In another non-limiting example, the nucleotide of Formula IIIa-IIIg or IVa-IVg described herein can be present at one or more of positions 1, 2, 3 and 4, counting from the 3′-end of the sense strand. In some embodiments, the nucleotide of Formula IIIa-IIIg or IVa-IVg can be present at one or more of positions 18, 19, 20 and 21, counting from 5′-end of the sense strand. The nucleotide of Formula IIIa-IIIg or IVa-IVg described herein can also be located at a central region of sense strand. For example, the nucleotide of Formula IIIa-IIIg or IVa-IVg described herein can be located at one or more of positions 6, 7, 8, 9, 10, 11, 12 and 13, counting from 5′-end of the sense strand. In some embodiments, the nucleotide of Formula IIIa-IIIg or IVa-IVg is at the 5-terminus of the sense strand.

In some embodiments, the antisense strand comprises 1, 2, 3, 4, 5, 6, 7, 8, 9, 10 or more of nucleotides of Formula IIIa-IIIg or IVa-IVg described herein. The nucleotide of Formula IIIa-IIIg or IVa-IVg described herein can be present at any position of the antisense strand. For example, the nucleotide of Formula IIIa-IIIg or IVa-IVg described herein can be present at a terminal region of the antisense strand. For example, the nucleotide of Formula IIIa-IIIg or IVa-IVg described herein can be present at one or more of positions 1, 2, 3 and 4, counting from the 5′-end of the antisense strand. In another non-limiting example, the nucleotide of Formula IIIa-IIIg or IVa-IVg described herein nucleotide can be present at one or more of positions 1, 2, 3, 4, 5 and 6, counting from the 3′-end of the antisense strand. In some embodiments, the nucleotide of Formula IIIa-IIIg or IVa-IVg described herein nucleotide can be present at one or more of positions 18, 19, 20, 21, 22 and 23, counting from 5′-end of the antisense strand. The nucleotide of Formula IIIa-IIIg or IVa-IVg described herein nucleotide can also be located at a central region of the antisense strand. For example, the nucleotide of Formula IIIa-IIIg or IVa-IVg described herein nucleotide can be located at one or more of positions 6, 7, 8, 9, 10, 11, 12 and 13, counting from 5′-end of the antisense strand. In some embodiments, the nucleotide of Formula IIIa-IIIg or IVa-IVg is at the 3′-terminus of the antisense strand.

As described herein, the dsRNA agent can comprise one or more, e.g., 1, 2, 3, 4, 5, 6, 7, 8, 9, 10 or more nucleotides comprising a modified sugar. Accordingly, in some embodiments, the dsRNA agent can comprise one or more, e.g., 1, 2, 3, 4, 5, 6, 7, 8, 9, 10 or more nucleotides independently selected from the group consisting of 2′-F, 2-OMe, acyclic nucleotides, locked nucleic acid (LNA), HNA, CeNA, 2′-methoxyethyl, 2′-O-allyl, 2′-C-allyl, 2′-O—N-methylacetamido (2′-O-NMA), a 2′-O-dimethylaminoethoxyethyl (2′-O-DMAEOE), 2′-O-aminopropyl (2′-O-AP), and 2′-ara-F. A nucleotide comprising modified sugar can be present anywhere in the dsRNA molecule. For example, a nucleotide comprising a modified sugar can be present in the sense strand or a nucleotide comprising a modified sugar can be present in the antisense strand. When two or more nucleotides comprising a modified sugar are present in the dsRNA molecule, they can all be in the sense strand, antisense strand or both in the sense and antisense strands.

As described herein, the dsRNA molecule described herein can comprise at least one, e.g., 1, 2, 3, 4, 5, 6, 7, 8, 9, 10 or more 2′-fluoro (2′-F) nucleotides. In some embodiments, the sense strand comprises 1, 2, 3, 4, 5, 6, 7, 8, 9, 10 or more 2′-fluoro nucleotides. The 2′-fluoro nucleotides can be located anywhere in the sense strand. For example, the sense strand comprises a 2′-fluoro nucleotide at position 10, counting from 5′-end of the sense strand. In some embodiments, the sense strand comprises a 2′-fluoro nucleotide at position 10, counting from 5′-end of the sense strand and the sense strand further comprises a 2′-fluoro nucleotide at position 8, 9, 11 or 12, counting from 5′-end of the sense strand. For example, the sense strand comprises a 2′-fluoro nucleotide at positions 9 10, counting from 5′-end of the sense strand. In another example, the sense strand comprises a 2′-fluoro nucleotide at positions 10 and 11, counting from 5′-end of the sense strand. In some embodiments, the sense strand comprises a 2′-fluoro nucleotide at positions 9, 10 and 11, counting from 5′-end of the sense strand. In some other embodiments, the sense strand comprises a 2′-fluoro nucleotide at positions 8, 9 and 10, counting from 5′-end of the sense strand. In yet some other embodiments, the sense strand comprises a 2′-fluoro nucleotide at positions 10, 11 and 12, counting from 5′-end of the sense strand.

In some embodiments, the antisense comprises 2′-fluoro nucleotides at positions 7, 10 and 11 from the 5′-end. In some other embodiments, the sense strand comprises 2′-fluoro nucleotides at positions 7, 9, 10 and 11 from the 5′-end. In some embodiments, the sense strand comprises 2′-fluoro nucleotides at positions opposite or complimentary to positions 11, 12 and 15 of the antisense strand, counting from the 5′-end of the antisense strand. In some other embodiments, the sense strand comprises 2′-fluoro nucleotides at positions opposite or complimentary to positions 11, 12, 13 and 15 of the antisense strand, counting from the 5′-end of the antisense strand. In some embodiments, the sense strand comprises a block of two, three or four 2′-fluoro nucleotides.

In some embodiments, the sense strand does not comprise a 2′-fluoro nucleotide in position opposite or complimentary to a thermally destabilizing modification of the duplex in the antisense strand.

In some embodiments, the antisense strand comprises 1, 2, 3, 4, 5, 6, 7, 8, 9, 10 or more 2′-fluoro nucleotides. The 2′-fluoro nucleotides can be located anywhere in the antisense strand. For example, the antisense strand can comprise a 2′-fluoro nucleotide at position 14, counting from 5′-end of the antisense strand. In some embodiments, the antisense comprises 2′-fluoro nucleotides at positions 2, 14 and 16, counting from the 5′-end of the antisense strand. In some other embodiments, the antisense comprises 2′-fluoro nucleotides at positions 2, 6, 14 and 16 from the 5′-end. In still some embodiments, the antisense comprises 2′-fluoro nucleotides at positions 2, 6, 8, 9, 14 and 16 from the 5′-end.

In some embodiments, the antisense strand comprises at least one 2′-fluoro nucleotide adjacent to a destabilizing modification. For example, the 2′-fluoro nucleotide can be the nucleotide at the 5′-end or the 3′-end of a destabilizing modification, i.e., at position -1 or +1 from the position of the destabilizing modification. In some embodiments, the antisense strand comprises a 2′-fluoro nucleotide at each of the 5′-end and the 3′-end of the destabilizing modification, i.e., positions -1 and +1 from the position of the destabilizing modification. In some embodiments, the antisense strand comprises at least two 2′-fluoro nucleotides at the 3′-end of the destabilizing modification, i.e., at positions +1 and +2 from the position of the destabilizing modification.

In some embodiments, both the sense and the antisense strands comprise at least one 2′-fluoro nucleotide. The 2′-fluoro modification can occur on any nucleotide of the sense strand or antisense strand. For instance, the 2′-fluoro modification can occur on every nucleotide on the sense strand and/or antisense strand; each 2′-fluoro modification can occur in an alternating pattern on the sense strand or antisense strand; or the sense strand or antisense strand comprises both 2′-fluoro modifications in an alternating pattern. The alternating pattern of the 2′-fluoro modifications on the sense strand may be the same or different from the antisense strand, and the alternating pattern of the 2′-fluoro modifications on the sense strand can have a shift relative to the alternating pattern of the 2′-fluoro modifications on the antisense strand.

As described herein, the dsRNA molecule described herein can comprise at least one, e.g., 1, 2, 3, 4, 5, 6, 7, 8, 9, 10 or more 2′-OMe nucleotides. Without limitations, the 2′-OMe nucleotides all can be present in one strand. The 2′-OMe nucleotide may occur on any nucleotide of the sense strand or antisense strand or both in any position of the strand.

In some embodiments, the sense strand comprises 1, 2, 3, 4, 5, 6, 7, 8, 9, 10 or more 2′-OMe nucleotides. The 2′-OMe nucleotides can be located anywhere in the sense strand. In some embodiments, the antisense strand comprises 1, 2, 3, 4, 5, 6, 7, 8, 9, 10 or more 2′-OMe nucleotides. The 2′-OMe nucleotides can be located anywhere in the antisense strand.

As described herein, the dsRNA molecule described herein can comprise at least one, e.g., 1, 2, 3, 4, 5, 6, 7, 8, 9, 10 or more 2′-deoxy, e.g., 2′-H ribose nucleotides. For example, the dsRNA can comprise 1, 2, 3, 4, 5, 6, 7, 8, 9 or 10 2′-deoxy, e.g., 2′-H nucleotides. The 2′-deoxy nucleotide may occur on any nucleotide of the sense strand or antisense strand or both in any position of the strand.

As described herein, the dsRNA can comprise at least one, e.g., at least two, at least three, at least four, at least five, at least six, at least seven or more, 2′-deoxy modifications in a central region of the sense strand and/or the antisense strand. For example, at least one of the sense stand and the antisense can comprise at least one, e.g., at least two, at least three, at least four, at least five, at least six, at least seven or more, 2′-deoxy modification in positions 5-17, e.g., positions 6-16, positions 6-15, positions 6-14, positions 6-13, positions 6-12, positions 7-15, positions 7-14, positions 7-13, positions, 7-12, positions 8-16, positions 8-15, positions 8-14, positions 8-13, positions 8-12, positions 9-16, positions 9-15, positions 9-14, positions 9-13, positions 9-12, positions 10-16, positions 10-15, positions 10-14, positions 10-13 or positions 10-12, counting from the 5′-end of the sense strand or the antisense strand.

In some embodiments, the antisense strand comprises 1, 2, 3, 4, 5 or 6 of 2′-deoxy nucleotides. For example, antisense strand can comprise 2, 3, 4, 5 or 6 of 2′-deoxy nucleotides. The 2′-deoxy nucleotides can be located anywhere in the antisense strand. For example, the antisense strand comprises a 2′-deoxy nucleotide at 1, 2, 3, 4, 5 or 6 of positions 2, 5, 7, 12, 14 and 16, counting from 5′-end of the antisense strand. In one non-limiting example, the antisense strand comprises a 2′-deoxy nucleotide at 1, 2, 3 or 4 of positions 2, 5, 7, and 12, counting from 5′-end of the antisense strand.

In some embodiments, the antisense comprises a 2′-deoxy nucleotide at positions 5 and 7, counting from 5′-end of the antisense strand. For example, the antisense strand comprises a 2′-deoxy nucleotide at positions 5, 7 and 12, counting from 5′-end of the antisense strand. In some embodiments, the antisense strand comprises a 2′-deoxy nucleotide at positions 2, 5 and 7, counting from 5′-end of the antisense strand. For example, the antisense strand comprises a 2′-deoxy nucleotide at positions 2, 5, 7 and 12, counting from 5′-end of the antisense strand. In some embodiments, the antisense strand comprises a 2′-deoxy nucleotide at positions 2, 5, 7, 12 and 14, counting, from 5′-end of the antisense strand. For example, the antisense strand comprises a 2′-deoxy nucleotide at positions 2, 5, 7, 12, 14 and 16, counting from 5′-end of the antisense strand

In some embodiments, the antisense comprises a 2′-deoxy nucleotide at position 2 or 12, counting from 5′-end of the antisense strand. For example, the antisense comprises a 2′-deoxy nucleotide at position 12, counting from 5′-end of the antisense strand.

In some embodiments, the dsRNA comprises at least three 2′-deoxy modifications, wherein the 2′-deoxy modifications are at positions 2 and 14 of the antisense strand, counting from 5′-end of the antisense strand, and at position 11 of the sense strand, counting from 5′-end of the sense strand.

In some embodiments, the dsRNA comprises at least five 2′-deoxy modifications, wherein the 2′-deoxy modifications are at positions 2, 12 and 14 of the antisense strand, counting from 5′-end of the antisense strand, and at positions 9 and 11 of the sense strand, counting from 5′-end of the sense strand.

In some embodiments, the dsRNA comprises at least seven 2′-deoxy modifications, wherein the 2′-deoxy modifications are at positions 2, 5, 7, 12 and 14 of the antisense strand, counting from 5′-end of the antisense strand, and at positions 9 and 11 of the sense strand, counting from 5′-end of the sense strand.

In some embodiments, the antisense strand comprises at least five 2′-deoxy modifications at positions 2, 5, 7, 12 and 14, counting from 5′-end of the antisense strand.

In one non-limiting example, the sense strand does not comprise a 2′-deoxy nucleotide at position 11, counting from 5′-end of the sense strand.

In some embodiments, the dsRNA can comprise one or more, e.g., 1, 2, 3, 4, 5, 6, 7, 8, 9, 10 or more nucleotides comprising a non-natural nucleobase

A nucleotide comprising a non-natural nucleobase can be present anywhere in the dsRNA molecule. For example, a nucleotide comprising a non-natural nucleobase can be present in the sense strand or a nucleotide comprising a non-natural nucleobase can be present in the antisense strand. When two or more nucleotides comprising a non-natural nucleobase are present in the dsRNA molecule, they can all be in the sense strand, antisense strand or both in the sense and antisense strands.

The dsRNA molecule described herein can further comprise at least one phosphorothioate or methylphosphonate internucleoside linkage. The phosphorothioate or methylphosphonate internucleoside linkage modification may occur on any nucleotide of the sense strand or antisense strand or both in any position of the strand. For instance, the internucleoside linkage modification may occur on every nucleotide on the sense strand and/or antisense strand; each internucleoside linkage modification may occur in an alternating pattern on the sense strand or antisense strand; or the sense strand or antisense strand comprises both internucleoside linkage modifications in an alternating pattern. The alternating pattern of the internucleoside linkage modification on the sense strand may be the same or different from the antisense strand, and the alternating pattern of the internucleoside linkage modification on the sense strand may have a shift relative to the alternating pattern of the internucleoside linkage modification on the antisense strand.

In some embodiments, the dsRNA molecule comprises the phosphorothioate or methylphosphonate internucleoside linkage modification in the overhang region. For example, the overhang region comprises two nucleotides having a phosphorothioate or methylphosphonate internucleoside linkage between the two nucleotides. Internucleoside linkage modifications also may be made to link the overhang nucleotides with the terminal paired nucleotides within duplex region. For example, at least 2, 3, 4, or all the overhang nucleotides may be linked through phosphorothioate or methylphosphonate internucleoside linkage, and optionally, there may be additional phosphorothioate or methylphosphonate internucleoside linkages linking the overhang nucleotide with a paired nucleotide that is next to the overhang nucleotide. For instance, there may be at least two phosphorothioate internucleoside linkages between the terminal three nucleotides, in which two of the three nucleotides are overhang nucleotides, and the third is a paired nucleotide next to the overhang nucleotide. Preferably, these terminal three nucleotides may be at the 3′-end of the antisense strand.

In some embodiments, the sense strand of the dsRNA molecule comprises 1-10 blocks of two to ten phosphorothioate or methylphosphonate internucleoside linkages separated by 1, 2, 3, 4, 5, 6, 7, 8, 9, 10, 11, 12, 13, 14, 15 or 16 phosphate internucleoside linkages, wherein one of the phosphorothioate or methylphosphonate internucleoside linkages is placed at any position in the oligonucleotide sequence and the said sense strand is paired with an antisense strand comprising any combination of phosphorothioate, methylphosphonate and phosphate internucleoside linkages or an antisense strand comprising either phosphorothioate or methylphosphonate or phosphate linkage.

In some embodiments, the antisense strand of the dsRNA molecule comprises two blocks of two phosphorothioate or methylphosphonate internucleoside linkages separated by 1, 2, 3, 4, 5, 6, 7, 8, 9, 10, 11, 12, 13, 14, 15, 16, 17, or 18 phosphate internucleoside linkages, wherein one of the phosphorothioate or methylphosphonate internucleoside linkages is placed at any position in the oligonucleotide sequence and the said antisense strand is paired with a sense strand comprising any combination of phosphorothioate, methylphosphonate and phosphate internucleoside linkages or an antisense strand comprising either phosphorothioate or methylphosphonate or phosphate linkage.

In some embodiments, the antisense strand of the dsRNA molecule comprises two blocks of three phosphorothioate or methylphosphonate internucleoside linkages separated by 1, 2, 3, 4, 5, 6, 7, 8, 9, 10, 11, 12, 13, 14, 15 or 16 phosphate internucleoside linkages, wherein one of the phosphorothioate or methylphosphonate internucleoside linkages is placed at any position in the oligonucleotide sequence and the said antisense strand is paired with a sense strand comprising any combination of phosphorothioate, methylphosphonate and phosphate internucleoside linkages or an antisense strand comprising either phosphorothioate or methylphosphonate or phosphate linkage.

In some embodiments, the antisense strand of the dsRNA molecule comprises two blocks of four phosphorothioate or methylphosphonate internucleoside linkages separated by 1, 2, 3, 4, 5, 6, 7, 8, 9, 10, 11, 12, 13 or 14 phosphate internucleoside linkages, wherein one of the phosphorothioate or methylphosphonate internucleoside linkages is placed at any position in the oligonucleotide sequence and the said antisense strand is paired with a sense strand comprising any combination of phosphorothioate, methylphosphonate and phosphate internucleoside linkages or an antisense strand comprising either phosphorothioate or methylphosphonate or phosphate linkage.

In some embodiments, the antisense strand of the dsRNA molecule comprises two blocks of five phosphorothioate or methylphosphonate internucleoside linkages separated by 1, 2, 3, 4, 5, 6, 7, 8, 9, 10, 11 or 12 phosphate internucleoside linkages, wherein one of the phosphorothioate or methylphosphonate internucleoside linkages is placed at any position in the oligonucleotide sequence and the said antisense strand is paired with a sense strand comprising any combination of phosphorothioate, methylphosphonate and phosphate internucleoside linkages or an antisense strand comprising either phosphorothioate or methylphosphonate or phosphate linkage.

In some embodiments, the antisense strand of the dsRNA molecule comprises two blocks of six phosphorothioate or methylphosphonate internucleoside linkages separated by 1, 2, 3, 4, 5, 6, 7, 8, 9 or 10 phosphate internucleoside linkages, wherein one of the phosphorothioate or methylphosphonate internucleoside linkages is placed at any position in the oligonucleotide sequence and the said antisense strand is paired with a sense strand comprising any combination of phosphorothioate, methylphosphonate and phosphate internucleoside linkages or an antisense strand comprising either phosphorothioate or methylphosphonate or phosphate linkage.

In some embodiments, the antisense strand of the dsRNA molecule comprises two blocks of seven phosphorothioate or methylphosphonate internucleoside linkages separated by 1, 2, 3, 4, 5, 6, 7 or 8 phosphate internucleoside linkages, wherein one of the phosphorothioate or methylphosphonate internucleoside linkages is placed at any position in the oligonucleotide sequence and the said antisense strand is paired with a sense strand comprising any combination of phosphorothioate, methylphosphonate and phosphate internucleoside linkages or an antisense strand comprising either phosphorothioate or methylphosphonate or phosphate linkage.

In some embodiments, the antisense strand of the dsRNA molecule comprises two blocks of eight phosphorothioate or methylphosphonate internucleoside linkages separated by 1, 2, 3, 4, 5 or 6 phosphate internucleoside linkages, wherein one of the phosphorothioate or methylphosphonate internucleoside linkages is placed at any position in the oligonucleotide sequence and the said antisense strand is paired with a sense strand comprising any combination of phosphorothioate, methylphosphonate and phosphate internucleoside linkages or an antisense strand comprising either phosphorothioate or methylphosphonate or phosphate linkage.

In some embodiments, the antisense strand of the dsRNA molecule comprises two blocks of nine phosphorothioate or methylphosphonate internucleoside linkages separated by 1, 2, 3 or 4 phosphate internucleoside linkages, wherein one of the phosphorothioate or methylphosphonate internucleoside linkages is placed at any position in the oligonucleotide sequence and the said antisense strand is paired with a sense strand comprising any combination of phosphorothioate, methylphosphonate and phosphate internucleoside linkages or an antisense strand comprising either phosphorothioate or methylphosphonate or phosphate linkage.

In some embodiments, the dsRNA molecule described herein further comprises one or more phosphorothioate or methylphosphonate internucleoside linkage modification within 1-10 of the termini position(s) of the sense and/or antisense strand. For example, at least 2, 3, 4, 5, 6, 7, 8, 9 or 10 nucleotides may be linked through phosphorothioate or methylphosphonate internucleoside linkage at one end or both ends of the sense and/or antisense strand.

In some embodiments, the dsRNA molecule described herein comprises one or more phosphorothioate or methylphosphonate internucleoside linkage modification within 1-10 of the internal region of the duplex of each of the sense and/or antisense strand. For example, at least 2, 3, 4, 5, 6, 7, 8, 9 or 10 nucleotides may be linked through phosphorothioate methylphosphonate internucleoside linkage at position 8-16 of the duplex region counting from the 5′-end of the sense strand; the dsRNA molecule can optionally further comprise one or more phosphorothioate or methylphosphonate internucleoside linkage modification within 1-10 of the termini position(s).

In some embodiments, the dsRNA molecule described herein further comprises one to five phosphorothioate or methylphosphonate internucleoside linkage modification(s) within position 1-5 and one to five phosphorothioate or methylphosphonate internucleoside linkage modification(s) within the last 3 positions of the sense strand (counting from the 5′-end), and one to five phosphorothioate or methylphosphonate internucleoside linkage modification at positions 1 and 2 and one to five phosphorothioate or methylphosphonate internucleoside linkage modification within the last six positions of the antisense strand (counting from the 5′-end).

In some embodiments, the dsRNA molecule described herein further comprises one phosphorothioate internucleoside linkage modification within position 1-5 and one phosphorothioate or methylphosphonate internucleoside linkage modification within the last six positions of the sense strand (counting from the 5′-end), and one phosphorothioate internucleoside linkage modification at positions 1 and 2 and two phosphorothioate or methylphosphonate internucleoside linkage modifications within the last six the last six positions of the antisense strand (counting from the 5′-end).

In some embodiments, the dsRNA molecule described herein further comprises two phosphorothioate internucleoside linkage modifications within position 1-5 and one phosphorothioate internucleoside linkage modification within the last six positions of the sense strand (counting from the 5′-end), and one phosphorothioate internucleoside linkage modification at positions 1 and 2 and two phosphorothioate internucleoside linkage modifications within the last six positions of the antisense strand (counting from the 5′-end).

In some embodiments, the dsRNA molecule described herein further comprises two phosphorothioate internucleoside linkage modifications within position 1-5 and two phosphorothioate internucleoside linkage modifications within the last four positions of the sense strand (counting from the 5′-end), and one phosphorothioate internucleoside linkage modification at positions 1 and 2 and two phosphorothioate internucleoside linkage modifications within the last six positions of the antisense strand (counting from the 5′-end).

In some embodiments, the dsRNA molecule described herein further comprises two phosphorothioate internucleoside linkage modifications within position 1-5 and two phosphorothioate internucleoside linkage modifications within the last four positions of the sense strand (counting from the 5′-end), and one phosphorothioate internucleoside linkage modification at positions 1 and 2 and one phosphorothioate internucleoside linkage modification within the last six positions of the antisense strand (counting from the 5′-end).

In some embodiments, the dsRNA molecule described herein further comprises one phosphorothioate internucleoside linkage modification within position 1-5 and one phosphorothioate internucleoside linkage modification within the last four positions of the sense strand (counting from the 5′-end), and two phosphorothioate internucleoside linkage modifications at positions 1 and 2 and two phosphorothioate internucleoside linkage modifications within the last six positions of the antisense strand (counting from the 5′-end).

In some embodiments, the dsRNA molecule described herein further comprises one phosphorothioate internucleoside linkage modification within position 1-5 and one within the last six positions of the sense strand (counting from the 5′-end), and two phosphorothioate internucleoside linkage modification at positions 1 and 2 and one phosphorothioate internucleoside linkage modification within the last six positions of the antisense strand (counting from the 5′-end).

In some embodiments, the dsRNA molecule described herein further comprises one phosphorothioate internucleoside linkage modification within position 1-5 (counting from the 5′-end) of the sense strand, and two phosphorothioate internucleoside linkage modifications at positions 1 and 2 and one phosphorothioate internucleoside linkage modification within the last six positions of the antisense strand (counting from the 5′-end).

In some embodiments, the dsRNA molecule described herein further comprises two phosphorothioate internucleoside linkage modifications within position 1-5 (counting from the 5′-end) of the sense strand, and one phosphorothioate internucleoside linkage modification at positions 1 and 2 and two phosphorothioate internucleoside linkage modifications within the last six positions of the antisense strand (counting from the 5′-end).

In some embodiments, the dsRNA molecule described herein further comprises two phosphorothioate internucleoside linkage modifications within position 1-5 and one within the last six positions of the sense strand (counting from the 5′-end), and two phosphorothioate internucleoside linkage modifications at positions 1 and 2 and one phosphorothioate internucleoside linkage modification within the last six positions of the antisense strand (counting from the 5′-end).

In some embodiments, the dsRNA molecule described herein further comprises two phosphorothioate internucleoside linkage modifications within position 1-5 and one phosphorothioate internucleoside linkage modification within the last six positions of the sense strand (counting from the 5′-end), and two phosphorothioate internucleoside linkage modifications at positions 1 and 2 and two phosphorothioate internucleoside linkage modifications within the last six positions of the antisense strand (counting from the 5′-end).

In some embodiments, the dsRNA molecule described herein further comprises two phosphorothioate internucleoside linkage modifications within position 1-5 and one phosphorothioate internucleoside linkage modification within the last six positions of the sense strand (counting from the 5′-end), and one phosphorothioate internucleoside linkage modification at positions 1 and 2 and two phosphorothioate internucleoside linkage modifications within the last six positions of the antisense strand (counting from the 5′-end).

In some embodiments, the dsRNA molecule described herein further comprises two phosphorothioate internucleoside linkage modifications at position 1 and 2, and two phosphorothioate internucleoside linkage modifications at position 20 and 21 of the sense strand (counting from the 5′-end), and one phosphorothioate internucleoside linkage modification at positions 1 and one at position 21 of the antisense strand (counting from the 5′-end).

In some embodiments, the dsRNA molecule described herein further comprises one phosphorothioate internucleoside linkage modification at position 1, and one phosphorothioate internucleoside linkage modification at position 21 of the sense strand (counting from the 5′-end), and two phosphorothioate internucleoside linkage modifications at positions 1 and 2 and two phosphorothioate internucleoside linkage modifications at positions 20 and 21 the antisense strand (counting from the 5′-end).

In some embodiments, the dsRNA molecule described herein further comprises two phosphorothioate internucleoside linkage modifications at position 1 and 2, and two phosphorothioate internucleoside linkage modifications at position 21 and 22 of the sense strand (counting from the 5′-end), and one phosphorothioate internucleoside linkage modification at positions 1 and one phosphorothioate internucleoside linkage modification at position 21 of the antisense strand (counting from the 5′-end).

In some embodiments, the dsRNA molecule described herein further comprises one phosphorothioate internucleoside linkage modification at position 1, and one phosphorothioate internucleoside linkage modification at position 21 of the sense strand (counting from the 5′-end), and two phosphorothioate internucleoside linkage modifications at positions 1 and 2 and two phosphorothioate internucleoside linkage modifications at positions 21 and 22 the antisense strand (counting from the 5′-end).

In some embodiments, the dsRNA molecule described herein further comprises two phosphorothioate internucleoside linkage modifications at position 1 and 2, and two phosphorothioate internucleoside linkage modifications at position 22 and 23 of the sense strand (counting from the 5′-end), and one phosphorothioate internucleoside linkage modification at positions 1 and one phosphorothioate internucleoside linkage modification at position 21 of the antisense strand (counting from the 5′-end).

In some embodiments, the dsRNA molecule described herein further comprises one phosphorothioate internucleoside linkage modification at position 1, and one phosphorothioate internucleoside linkage modification at position 21 of the sense strand (counting from the 5′-end), and two phosphorothioate internucleoside linkage modifications at positions 1 and 2 and two phosphorothioate internucleoside linkage modifications at positions 22 and 23 the antisense strand (counting from the 5′-end).

In some embodiments, the sense strand comprises at least two phosphorothioate internucleoside linkages between the first five nucleotides counting from the 5′ end of the sense strand. For example, the sense strand comprises phosphorothioate linkages between nucleotides 1 and 2, and between nucleotides 2 and 3, counting from 5′-end of the sense strand.

In some embodiments, the antisense strand comprises at least two phosphorothioate internucleoside linkages between the first five nucleotides counting from the 5′-end of the antisense strand. For example, the antisense strand comprises phosphorothioate linkages between nucleotides 1 and 2, and between nucleotides 2 and 3, counting from 5′-end of the antisense strand.

In some embodiments, the antisense strand comprises at least two phosphorothioate internucleoside linkages between the first five nucleotides counting from the 3′ end of the antisense strand. For example, the antisense strand comprises phosphorothioate linkages between nucleotides n and n−1, and between nucleotides n−1 and n−2, where n is length of the antisense strand, i.e., number of nucleotides in the antisense strand. In other words, the antisense strand comprises phosphorothioate linkages between nucleotides 1 and 2, and between nucleotides 2 and 3, counting from 3′-end of the antisense strand.

In some embodiments, the antisense strand comprises at least two phosphorothioate internucleoside linkages between the first five nucleotides counting from the 5′-end of the antisense strand and at least two phosphorothioate internucleoside linkages between the first five nucleotides counting from the 5′-end of the antisense strand. For example, the antisense strand comprises phosphorothioate linkages between nucleotides 1 and 2, and between nucleotides 2 and 3, counting from 5′-end of the antisense strand and between nucleotides 1 and 2, and between nucleotides 2 and 3, counting from 3′-end of the antisense strand.

In some embodiments, the sense strand comprises at least two phosphorothioate internucleoside linkages between the first five nucleotides counting from the 5′ end of the sense strand and the antisense strand comprises at least two phosphorothioate internucleoside linkages between the first five nucleotides counting from the 5′-end of the antisense strand. For example, the sense strand comprises phosphorothioate linkages between nucleotides 1 and 2, and between nucleotides 2 and 3, counting from 5′-end of the sense strand, and the antisense strand comprises phosphorothioate linkages between nucleotides 1 and 2, and between nucleotides 2 and 3, counting from 5′-end of the antisense strand.

In some embodiments, the sense strand comprises at least two phosphorothioate internucleoside linkages between the first five nucleotides counting from the 5′ end of the sense strand and the antisense strand comprises at least two phosphorothioate internucleoside linkages between the first five nucleotides counting from the 3′-end of the antisense strand. For example, the sense strand comprises phosphorothioate linkages between nucleotides 1 and 2, and between nucleotides 2 and 3, counting from 5′-end of the sense strand, and the antisense strand comprises phosphorothioate linkages between nucleotides 1 and 2, and between nucleotides 2 and 3, counting from 3′-end of the antisense strand.

In some embodiments, dsRNA molecule described herein comprises a pattern of backbone chiral centers. In some embodiments, a common pattern of backbone chiral centers comprises at least 5 internucleotidic linkages in the Sp configuration. In some embodiments, a common pattern of backbone chiral centers comprises at least 6 internucleotidic linkages in the Sp configuration. In some embodiments, a common pattern of backbone chiral centers comprises at least 7 internucleotidic linkages in the Sp configuration. In some embodiments, a common pattern of backbone chiral centers comprises at least 8 internucleotidic linkages in the Sp configuration. In some embodiments, a common pattern of backbone chiral centers comprises at least 9 internucleotidic linkages in the Sp configuration. In some embodiments, a common pattern of backbone chiral centers comprises at least 10 internucleotidic linkages in the Sp configuration. In some embodiments, a common pattern of backbone chiral centers comprises at least 11 internucleotidic linkages in the Sp configuration. In some embodiments, a common pattern of backbone chiral centers comprises at least 12 internucleotidic linkages in the Sp configuration. In some embodiments, a common pattern of backbone chiral centers comprises at least 13 internucleotidic linkages in the Sp configuration. In some embodiments, a common pattern of backbone chiral centers comprises at least 14 internucleotidic linkages in the Sp configuration. In some embodiments, a common pattern of backbone chiral centers comprises at least 15 internucleotidic linkages in the Sp configuration. In some embodiments, a common pattern of backbone chiral centers comprises at least 16 internucleotidic linkages in the Sp configuration. In some embodiments, a common pattern of backbone chiral centers comprises at least 17 internucleotidic linkages in the Sp configuration. In some embodiments, a common pattern of backbone chiral centers comprises at least 18 internucleotidic linkages in the Sp configuration. In some embodiments, a common pattern of backbone chiral centers comprises at least 19 internucleotidic linkages in the Sp configuration. In some embodiments, a common pattern of backbone chiral centers comprises no more than 8 internucleotidic linkages in the Rp configuration. In some embodiments, a common pattern of backbone chiral centers comprises no more than 7 internucleotidic linkages in the Rp configuration. In some embodiments, a common pattern of backbone chiral centers comprises no more than 6 internucleotidic linkages in the Rp configuration. In some embodiments, a common pattern of backbone chiral centers comprises no more than 5 internucleotidic linkages in the Rp configuration. In some embodiments, a common pattern of backbone chiral centers comprises no more than 4 internucleotidic linkages in the Rp configuration. In some embodiments, a common pattern of backbone chiral centers comprises no more than 3 internucleotidic linkages in the Rp configuration. In some embodiments, a common pattern of backbone chiral centers comprises no more than 2 internucleotidic linkages in the Rp configuration. In some embodiments, a common pattern of backbone chiral centers comprises no more than 1 internucleotidic linkages in the Rp configuration. In some embodiments, a common pattern of backbone chiral centers comprises no more than 8 internucleotidic linkages which are not chiral (as a non-limiting example, a phosphodiester). In some embodiments, a common pattern of backbone chiral centers comprises no more than 7 internucleotidic linkages which are not chiral. In some embodiments, a common pattern of backbone chiral centers comprises no more than 6 internucleotidic linkages which are not chiral. In some embodiments, a common pattern of backbone chiral centers comprises no more than 5 internucleotidic linkages which are not chiral. In some embodiments, a common pattern of backbone chiral centers comprises no more than 4 internucleotidic linkages which are not chiral. In some embodiments, a common pattern of backbone chiral centers comprises no more than 3 internucleotidic linkages which are not chiral. In some embodiments, a common pattern of backbone chiral centers comprises no more than 2 internucleotidic linkages which are not chiral. In some embodiments, a common pattern of backbone chiral centers comprises no more than 1 internucleotidic linkages which are not chiral. In some embodiments, a common pattern of backbone chiral centers comprises at least 10 internucleotidic linkages in the Sp configuration, and no more than 8 internucleotidic linkages which are not chiral. In some embodiments, a common pattern of backbone chiral centers comprises at least 11 internucleotidic linkages in the Sp configuration, and no more than 7 internucleotidic linkages which are not chiral. In some embodiments, a common pattern of backbone chiral centers comprises at least 12 internucleotidic linkages in the Sp configuration, and no more than 6 internucleotidic linkages which are not chiral. In some embodiments, a common pattern of backbone chiral centers comprises at least 13 internucleotidic linkages in the Sp configuration, and no more than 6 internucleotidic linkages which are not chiral. In some embodiments, a common pattern of backbone chiral centers comprises at least 14 internucleotidic linkages in the Sp configuration, and no more than 5 internucleotidic linkages which are not chiral. In some embodiments, a common pattern of backbone chiral centers comprises at least 15 internucleotidic linkages in the Sp configuration, and no more than 4 internucleotidic linkages which are not chiral. In some embodiments, the internucleotidic linkages in the Sp configuration are optionally contiguous or not contiguous. In some embodiments, the internucleotidic linkages in the Rp configuration are optionally contiguous or not contiguous. In some embodiments, the internucleotidic linkages which are not chiral are optionally contiguous or not contiguous.

In some embodiments, dsRNA molecule described herein comprises a block is a stereochemistry block. In some embodiments, a block is an Rp block in that each internucleotidic linkage of the block is Rp. In some embodiments, a 5′-block is an Rp block. In some embodiments, a 3′-block is an Rp block. In some embodiments, a block is an Sp block in that each internucleotidic linkage of the block is Sp. In some embodiments, a 5′-block is an Sp block. In some embodiments, a 3′-block is an Sp block. In some embodiments, provided oligonucleotides comprise both Rp and Sp blocks. In some embodiments, provided oligonucleotides comprise one or more Rp but no Sp blocks. In some embodiments, provided oligonucleotides comprise one or more Sp but no Rp blocks. In some embodiments, provided oligonucleotides comprise one or more PO blocks wherein each internucleotidic linkage in a natural phosphate linkage.

In some embodiments, dsRNA molecule described herein comprises a 5′-block is an Sp block wherein each sugar moiety comprises a 2′-fluoro modification. In some embodiments, a 5′-block is an Sp block wherein each of internucleotidic linkage is a modified internucleotidic linkage and each sugar moiety comprises a 2′-fluoro modification. In some embodiments, a 5′-block is an Sp block wherein each of internucleoside linkage is a phosphorothioate linkage and each sugar moiety comprises a 2′-fluoro modification. In some embodiments, a 5′-block comprises 4 or more nucleoside units. In some embodiments, a 5′-block comprises 5 or more nucleoside units. In some embodiments, a 5′-block comprises 6 or more nucleoside units. In some embodiments, a 5′-block comprises 7 or more nucleoside units. In some embodiments, a 3′-block is an Sp block wherein each sugar moiety comprises a 2′-fluoro modification. In some embodiments, a 3′-block is an Sp block wherein each of internucleotidic linkage is a modified internucleotidic linkage and each sugar moiety comprises a 2′-fluoro modification. In some embodiments, a 3′-block is an Sp block wherein each of internucleotidic linkage is a phosphorothioate linkage and each sugar moiety comprises a 2′-fluoro modification. In some embodiments, a 3′-block comprises 4 or more nucleoside units. In some embodiments, a 3′-block comprises 5 or more nucleoside units. In some embodiments, a 3′-block comprises 6 or more nucleoside units. In some embodiments, a 3′-block comprises 7 or more nucleoside units.

In some embodiments, dsRNA molecule described herein comprises a type of nucleoside in a region or an oligonucleotide is followed by a specific type of internucleotidic linkage, e.g., natural phosphate linkage, modified internucleotidic linkage, Rp chiral internucleotidic linkage, Sp chiral internucleotidic linkage, etc. In some embodiments, A is followed by Sp. In some embodiments, A is followed by Rp. In some embodiments, A is followed by natural phosphate linkage (PO). In some embodiments, U is followed by Sp. In some embodiments, U is followed by Rp. In some embodiments, U is followed by natural phosphate linkage (PO). In some embodiments, C is followed by Sp. In some embodiments, C is followed by Rp. In some embodiments, C is followed by natural phosphate linkage (PO). In some embodiments, G is followed by Sp. In some embodiments, G is followed by Rp. In some embodiments, G is followed by natural phosphate linkage (PO). In some embodiments, C and U are followed by Sp. In some embodiments, C and U are followed by Rp. In some embodiments, C and U are followed by natural phosphate linkage (PO). In some embodiments, A and G are followed by Sp. In some embodiments, A and G are followed by Rp.

Various publications describe multimeric siRNA which can all be used with the oligonucleotide and dsRNA of the invention. Such publications include WO2007/091269, U.S. Pat. No. 7,858,769, WO2010/141511, WO2007/117686, WO2009/014887 and WO2011/031520 which are hereby incorporated by their entirely.

In some embodiments, the dsRNA molecule described herein comprises one or more overhang regions and/or capping groups of dsRNA molecule at the 3′-end, or 5′-end or both ends of a strand. The overhang can be 1-10 nucleotides in length. For example, the overhang can be 1, 2, 3, 4, 5, 6, 7, 8, 9 or 10 nucleotides in length. In some embodiments, the overhang is 1-6 nucleotides in length, for instance 2-6 nucleotides in length, 1-5 nucleotides in length, 2-5 nucleotides in length, 1-4 nucleotides in length, 2-4 nucleotides in length, 1-3 nucleotides in length, 2-3 nucleotides in length, or 1-2 nucleotides in length. The overhangs can be the result of one strand being longer than the other, or the result of two strands of the same length being staggered. The overhang can form a mismatch with the target sequence or it can be complementary to the gene sequences being targeted or it can be the other sequence. The first and second strands can also be joined, e.g., by additional bases to form a hairpin, or by other non-base linkers.

In some embodiments, the nucleotides in the overhang region of the dsRNA molecule described herein can each independently be a modified or unmodified nucleotide including, but not limited to 2′-sugar modified, such as, 2′-Fluoro 2′-O-methyl, thymidine (T), 2′-O-methoxyethyl-5-methyluridine, 2′-O-methoxyethyladenosine, 2′-O-methoxyethyl-5-methylcytidine, GNA, SNA, hGNA, hhGNA, mGNA, TNA, h′GNA, and any combinations thereof. For example, dTdT can be an overhang sequence for either end on either strand. The overhang can form a mismatch with the target mRNA or it can be complementary to the gene sequences being targeted or can be other sequence.

The 5′- or 3′-overhangs at the sense strand, antisense strand or both strands of the dsRNA molecule described herein may be phosphorylated. In some embodiments, the overhang region contains two nucleotides having a phosphorothioate between the two nucleotides, where the two nucleotides can be the same or different. In some embodiments, the overhang is present at the 3′-end of the sense strand, antisense strand or both strands. In some embodiments, this 3′-overhang is present in the antisense strand. In some embodiments, this 3′-overhang is present in the sense strand.

The dsRNA molecule described herein may comprise only a single overhang, which can strengthen the interference activity of the dsRNA, without affecting its overall stability. For example, the single-stranded overhang is located at the 3-terminal end of the sense strand or, alternatively, at the 3-terminal end of the antisense strand. The dsRNA can also have a blunt end, located at the 5′-end of the antisense strand (or the 3′-end of the sense strand) or vice versa.

Generally, the antisense strand of the dsRNA has a nucleotide overhang at the 3′-end, and the 5′-end is blunt. While not bound by theory, the asymmetric blunt end at the 5′-end of the antisense strand and 3′-end overhang of the antisense strand favor the guide strand loading into RISC process. For example, the single overhang is at least one, two, three, four, five, six, seven, eight, nine, or ten nucleotides in length. In some embodiments, the dsRNA has a 2 nucleotide overhang on the 3′-end of the antisense strand and a blunt end at the 5′-end of the antisense strand.

The dsRNA described herein can comprise one or more modified nucleotides. For example, every nucleotide in the sense strand and antisense strand of the dsRNA molecule can be modified. Each nucleotide can be modified with the same or different modification which can include one or more alteration of one or both of the non-linking phosphate oxygens and/or of one or more of the linking phosphate oxygens; alteration of a constituent of the ribose sugar; replacement of the ribose sugar; wholesale replacement of the phosphate moiety with “dephospho” linkers; modification or replacement of a naturally occurring base; and replacement or modification of the ribose-phosphate backbone.

As nucleic acids are polymers of subunits, many of the modifications occur at a position which is repeated within a nucleic acid, e.g., a modification of a base, or a phosphate moiety, or a non-linking O of a phosphate moiety. In some cases, the modification will occur at all of the subject positions in the nucleic acid but in many cases it will not. By way of example, a modification may only occur at a 3′ or 5′ terminal position, may only occur in a central region, may only occur at a non-terminal region, or may only occur in a terminal region, e.g., at a position on a terminal nucleotide or in the last 2, 3, 4, 5, or 10 nucleotides of a strand. A modification may occur in a double strand region, a single strand region, or in both. A modification may occur only in the double strand region of a RNA or may only occur in a single strand region of a RNA. For example, a phosphorothioate modification at a non-linking O position may only occur at one or both termini, may only occur in a terminal region, e.g., at a position on a terminal nucleotide or in the last 2, 3, 4, 5, or 10 nucleotides of a strand, or may occur in double strand and single strand regions, particularly at termini. The 5′ end or ends can be phosphorylated.

It may be possible, e.g., to enhance stability, to include particular bases in overhangs, or to include modified nucleotides or nucleotide surrogates, in single strand overhangs, e.g., in a 5′ or 3′ overhang, or in both. For example, it can be desirable to include purine nucleotides in overhangs. In some embodiments all or some of the bases in a 3′ or 5′ overhang may be modified, e.g., with a modification described herein. Modifications can include, e.g., the use of modifications at the 2′ position of the ribose sugar with modifications that are known in the art, e.g., the use of deoxyribonucleotides, 2′-deoxy-2′-fluoro (2′-F) or 2′-O-methyl modified instead of the ribosugar of the nucleobase, and modifications in the phosphate group, e.g., phosphorothioate modifications. Overhangs need not be homologous with the target sequence.

In some embodiments, the dsRNA molecule described herein comprises modifications of an alternating pattern, particular in the B1, B2, B3, B1′, B2′, B3′, B4′ regions. The term “alternating motif” or “alternative pattern” as used herein refers to a motif having one or more modifications, each modification occurring on alternating nucleotides of one strand. The alternating nucleotide may refer to one per every other nucleotide or one per every three nucleotides, or a similar pattern. For example, if A, B and C each represent one type of modification to the nucleotide, the alternating motif can be “ABABABABABAB . . . ,” “AABBAABBAABB . . . ,” “AABAABAABAAB . . . ,” “AAABAAABAAAB . . . ,” “AAABBBAAABBB . . . ,” or “ABCABCABCABC . . . ,” etc.

The type of modifications contained in the alternating motif may be the same or different. For example, if A, B, C, D each represent one type of modification on the nucleotide, the alternating pattern, i.e., modifications on every other nucleotide, may be the same, but each of the sense strand or antisense strand can be selected from several possibilities of modifications within the alternating motif such as “ABABAB . . . ”, “ACACAC . . . ” “BDBDBD . . . ” or “CDCDCD . . . ,” etc.

In some embodiments, the dsRNA molecule described herein comprises the modification pattern for the alternating motif on the sense strand relative to the modification pattern for the alternating motif on the antisense strand is shifted. The shift may be such that the modified group of nucleotides of the sense strand corresponds to a differently modified group of nucleotides of the antisense strand and vice versa. For example, the sense strand when paired with the antisense strand in the dsRNA duplex, the alternating motif in the sense strand may start with “ABABAB” from 5′-3′ of the strand and the alternating motif in the antisense strand may start with “BABABA” from 3′-5′ of the strand within the duplex region. As another example, the alternating motif in the sense strand may start with “AABBAABB” from 5′-3′ of the strand and the alternating motif in the antisense strand may start with “BBAABBAA” from 3′-5′ of the strand within the duplex region, so that there is a complete or partial shift of the modification patterns between the sense strand and the antisense strand.

In some embodiments of any one of the aspects described herein, the oligonucleotides described herein or at least one e.g., both strand of a dsRNA described herein are 5′ phosphorylated or include a phosphoryl analog at the 5′ prime terminus. 5′-phosphate modifications include those which are compatible with RISC mediated gene silencing. Suitable modifications include: 5′-monophosphate ((HO)₂(O)P—O-5′); 5′-diphosphate ((HO)₂(O)P—O—P(HO)(O)—O-5′); 5′-triphosphate ((HO)₂(O)P—O—(HO)(O)P—O—P(HO)(O)—O-5′); 5′-guanosine cap (7-methylated or non-methylated) (7m-G-O-5′-(HO)(O)P—O—(HO)(O)P—O—P(HO)(O)—O-5′); 5′-adenosine cap (Appp), and any modified or unmodified nucleotide cap structure (N—O-5′-(HO)(O)P—O—(HO)(O)P—O—P(HO)(O)—O-5′); 5′-monothiophosphate (phosphorothioate; (HO)₂(S)P—O-5′); 5′-monodithiophosphate (phosphorodithioate; (HO)(HS)(S)P—O-5′), 5′-phosphorothiolate ((HO)2(O)P—S-5′); any additional combination of oxygen/sulfur replaced monophosphate, diphosphate and triphosphates (e.g. 5′-alpha-thiotriphosphate, 5′-gamma-thiotriphosphate, etc.), 5′-phosphoramidates ((HO)₂(O)P—NH-5′, (HO)(NH₂)(O)P—O-5′), 5′-alkylphosphonates (e.g., RP(OH)(O)—O-5′-, R=alkyl, e.g., methyl, ethyl, isopropyl, propyl, etc.), 5′-alkenylphosphonates (i.e. vinyl, substituted vinyl, e.g., OH)₂(O)P-5′-CH═ or (OH)₂(O)P-5′-CH₂—), 5′-alkyletherphosphonates (e.g., R(OH)(O)P—O-5′, R=alkylether, e.g., methoxymethyl (MeOCH2-), ethoxymethyl, etc.) Other exemplary 5′-modifications include where Z is optionally substituted alkyl at least once, e.g., ((HO)₂(X)P—O[—(CH₂)ₐ—O—P(X)(OH)—O]_{b}-5′, ((HO)₂(X)P—O[—(CH₂)ₐ—P(X)(OH)—O]_{b}-5′, ((HO)₂(X)P—[—(CH₂)ₐ—O—P(X)(OH)—O]_{b}-5′; dialkyl terminal phosphates and phosphate mimics: HO[—(CH₂)ₐ—O—P(X)(OH)—O]_{b}-5′, H₂N[—(CH₂)ₐ—O—P(X)(OH)—O]_{b}-5′, H[—(CH₂)ₐ—O—P(X)(OH)—O]_{b}-5′, Me₂N[—(CH₂)ₐ—O—P(X)(OH)—O]_{b}-5′, HO[—(CH₂)ₐ—P(X)(OH)—O]_{b}-5′, H₂N[—(CH₂)ₐ—P(X)(OH)—O]_{b}-5′, H[—(CH₂)ₐ—P(X)(OH)—O]_{b}-5′, Me₂N[—(CH₂)ₐ—P(X)(OH)—O]_{b}-5′, wherein a and b are each independently 1-10. Other embodiments, include replacement of oxygen and/or sulfur with BH₃, BH₃⁻ and/or Se.

In some embodiments of any one of the aspects described herein, the oligonucleotide or at least one (e.g., both) strand of a dsRNA described herein comprises a 5′-vinylphosphonate group. For example, the oligonucleotide or at least one (e.g., both) strand of a dsRNA described herein comprises a 5′-E-vinyl or at least one (e.g., both) strand of a dsRNA described herein phosphonate group. In some other non-limiting example, the oligonucleotide comprises a 5′-Z-vinylphosphonate group.

In one example, the 5′-modification can be placed in the antisense strand of a double-stranded nucleic acid, e.g., dsRNA molecule. For example, the antisense comprises a 5′-E-vinylphosphonate. In some other non-limiting example, the antisense strand comprises a 5′-Z-vinylphosphonate group.

In some embodiments, the sense strand comprises a 5′-morpholino, a 5′-dimethylamino, a 5′-deoxy, an inverted abasic, or an inverted abasic locked nucleic acid modification at the 5′-end.

The dsRNA agents of the invention can comprise thermally destabilizing modifications in the seed region of the antisense strand (i.e., at positions 2-9 of the 5′-end of the antisense strand) to reduce or inhibit off-target gene silencing. Without wishing to be bound by a theory, dsRNAs with an antisense strand comprising at least one thermally destabilizing modification of the duplex within the first 9 nucleotide positions, counting from the 5′ end, of the antisense strand have reduced off-target gene silencing activity. Accordingly, in some embodiments, the antisense strand comprises at least one (e.g., one, two, three, four, five or more) thermally destabilizing modification of the duplex within the first 9 nucleotide positions of the 5′ region of the antisense strand. In some embodiments, thermally destabilizing modification of the duplex is located in positions 2-9, or preferably positions 4-8, from the 5′-end of the antisense strand. In some further embodiments, the thermally destabilizing modification of the duplex is located at position 5, 6, 7 or 8 from the 5′-end of the antisense strand.

In still some further embodiments, the thermally destabilizing modification of the duplex is located at position 7 from the 5′-end of the antisense strand.

In addition to the antisense strand comprising a thermally destabilizing modification, the dsRNA can also comprise one or more stabilizing modifications. For example, the dsRNA can comprise at least two (e.g., two, three, four, five, six, seven, eight, nine, ten or more) stabilizing modifications. Without limitations, the stabilizing modifications all can be present in one strand. In some embodiments, both the sense and the antisense strands comprise at least two stabilizing modifications. The stabilizing modification can occur on any nucleotide of the sense strand or antisense strand. For instance, the stabilizing modification can occur on every nucleotide on the sense strand and/or antisense strand; each stabilizing modification can occur in an alternating pattern on the sense strand or antisense strand; or the sense strand or antisense strand comprises both stabilizing modification in an alternating pattern. The alternating pattern of the stabilizing modifications on the sense strand may be the same or different from the antisense strand, and the alternating pattern of the stabilizing modifications on the sense strand can have a shift relative to the alternating pattern of the stabilizing modifications on the antisense strand.

In some embodiments, the antisense strand comprises at least two (e.g., two, three, four, five, six, seven, eight, nine, ten or more) stabilizing modifications. Without limitations, a stabilizing modification in the antisense strand can be present at any positions. In some embodiments, the antisense comprises stabilizing modifications at positions 2, 6, 8, 9, 14 and 16 from the 5′-end. In some other embodiments, the antisense comprises stabilizing modifications at positions 2, 6, 14 and 16 from the 5′-end. In still some other embodiments, the antisense comprises stabilizing modifications at positions 2, 14 and 16 from the 5′-end.

In some embodiments, the antisense strand comprises at least one stabilizing modification adjacent to the destabilizing modification. For example, the stabilizing modification can be the nucleotide at the 5′-end or the 3′-end of the destabilizing modification, i.e., at position −1 or +1 from the position of the destabilizing modification. In some embodiments, the antisense strand comprises a stabilizing modification at each of the 5′-end and the 3′-end of the destabilizing modification, i.e., positions -1 and +1 from the position of the destabilizing modification.

In some embodiments, the antisense strand comprises at least two stabilizing modifications at the 3′-end of the destabilizing modification, i.e., at positions +1 and +2 from the position of the destabilizing modification. In some embodiments, the sense strand comprises at least two (e.g., two, three, four, five, six, seven, eight, nine, ten or more) stabilizing modifications. Without limitations, a stabilizing modification in the sense strand can be present at any positions. In some embodiments, the sense strand comprises stabilizing modifications at positions 7, 10 and 11 from the 5′-end. In some other embodiments, the sense strand comprises stabilizing modifications at positions 7, 9, 10 and 11 from the 5′-end. In some embodiments, the sense strand comprises stabilizing modifications at positions opposite or complimentary to positions 11, 12 and 15 of the antisense strand, counting from the 5′-end of the antisense strand. In some other embodiments, the sense strand comprises stabilizing modifications at positions opposite or complimentary to positions 11, 12, 13 and 15 of the antisense strand, counting from the 5′-end of the antisense strand. In some embodiments, the sense strand comprises a block of two, three or four stabilizing modifications.

In some embodiments, the sense strand does not comprise a stabilizing modification in position opposite or complimentary to the thermally destabilizing modification of the duplex in the antisense strand.

It is noted a thermally stabilizing modification can replace a 2′-fluoro nucleotide in the sense and/or antisense strand. For example, a 2′-fluoro nucleotide at positions 8, 9, 10, 11 and/or 12, counting from 5′-end, of the sense strand, can be replaced with a thermally stabilizing modification. Similarly, a 2′-fluoro nucleotide at position 14, counting from 5′-end, of the antisense strand, can be replaced with a thermally stabilizing modification.

For the dsRNA molecules to be more effective in vivo, the antisense strand must have some metabolic stability. In other words, for the dsRNA molecules to be more effective in vivo, some amount of the antisense stand may need to be present in vivo after a period time after administration. Accordingly, in some embodiments, at least 40%, for example at least 45%, at least 50%, at least 55%, at least 60%, at least 65%, at least 70%, at least 75%, or at least 80% of the antisense strand of the dsRNA is present in vivo, for example in mouse liver, at day 5 after in vivo administration. In some embodiments, at least 40%, for example at least 45%, at least 50%, at least 55%, at least 60%, at least 65%, at least 70%, at least 75%, or at least 80% of the antisense strand of the dsRNA is present in vivo, for example in mouse liver, at day 6 after in vivo administration. In some embodiments, at least 40%, for example at least 45%, at least 50%, at least 55%, at least 60%, at least 65%, at least 70%, at least 75%, or at least 80% of the antisense strand of the dsRNA is present in vivo, for example in mouse liver, at day 7 after in vivo administration. In some embodiments, at least 40%, for example at least 45%, at least 50%, at least 55%, at least 60%, at least 65%, at least 70%, at least 75%, or at least 80% of the antisense strand of the dsRNA is present in vivo, for example in mouse liver, at day 8 after in vivo administration. In some embodiments, at least 40%, for example at least 45%, at least 50%, at least 55%, at least 60%, at least 65%, at least 70%, at least 75%, or at least 80% of the antisense strand of the dsRNA is present in vivo, for example in mouse liver, at day 9 after in vivo administration. In some embodiments, at least 40%, for example at least 45%, at least 50%, at least 55%, at least 60%, at least 65%, at least 70%, at least 75%, or at least 80% of the antisense strand of the dsRNA is present in vivo, for example in mouse liver, at day 10 after in vivo administration. In some embodiments, at least 40%, for example at least 45%, at least 50%, at least 55%, at least 60%, at least 65%, at least 70%, at least 75%, or at least 80% of the antisense strand of the dsRNA is present in vivo, for example in mouse liver, at day 11 after in vivo administration. In some embodiments, at least 40%, for example at least 45%, at least 50%, at least 55%, at least 60%, at least 65%, at least 70%, at least 75%, or at least 80% of the antisense strand of the dsRNA is present in vivo, for example in mouse liver, at day 12 after in vivo administration. In some embodiments, at least 40%, for example at least 45%, at least 50%, at least 55%, at least 60%, at least 65%, at least 70%, at least 75%, or at least 80% of the antisense strand of the dsRNA is present in vivo, for example in mouse liver, at day 13 after in vivo administration. In some embodiments, at least 40%, for example at least 45%, at least 50%, at least 55%, at least 60%, at least 65%, at least 70%, at least 75%, or at least 80% of the antisense strand of the dsRNA is present in vivo, for example in mouse liver, at day 14 after in vivo administration. In some embodiments, at least 40%, for example at least 45%, at least 50%, at least 55%, at least 60%, at least 65%, at least 70%, at least 75%, or at least 80% of the antisense strand of the dsRNA is present in vivo, for example in mouse liver, at day 15 after in vivo administration.

Uses of Oligonucleotides and dsRNAs

In some embodiments of any one of the aspects, the oligonucleotide described herein or the antisense strand of the dsRNA molecule described herein comprises a nucleotide sequence substantially complementary to a target nucleic acid, e.g., a target gene or mRNA.

Accordingly, in another aspect, the disclosure is directed to a use of an oligonucleotide and/or dsRNA molecule described herein for inhibiting expression of a target gene. In some embodiments, the present invention further relates to a use of an oligonucleotide and/or dsRNA molecule described herein for inhibiting expression of a target gene in vitro.

In another aspect, the disclosure is directed to a use of an oligonucleotide and/or dsRNA molecule described herein for use in inhibiting expression of a target gene in a subject. The subject may be any animal, such as a mammal, e.g., a mouse, a rat, a sheep, a cattle, a dog, a cat, or a human

In some embodiments, the oligonucleotide and/or dsRNA molecule described herein is administered in buffer.

In some embodiments, oligonucleotide and/or dsRNA molecule described herein described herein can be formulated for administration to a subject. A formulated oligonucleotide and/or dsRNA composition can assume a variety of states. In some examples, the composition is at least partially crystalline, uniformly crystalline, and/or anhydrous (e.g., less than 80, 50, 30, 20, or 10% water). In another example, the siRNA is in an aqueous phase, e.g., in a solution that includes water.

The aqueous phase or the crystalline compositions can, e.g., be incorporated into a delivery vehicle, e.g., a liposome (particularly for the aqueous phase) or a particle (e.g., a microparticle as can be appropriate for a crystalline composition). Generally, the siRNA composition is formulated in a manner that is compatible with the intended method of administration, as described herein. For example, in particular embodiments the composition is prepared by at least one of the following methods: spray drying, lyophilization, vacuum drying, evaporation, fluid bed drying, or a combination of these techniques; or sonication with a lipid, freeze-drying, condensation and other self-assembly.

A oligonucleotide and/or dsRNA preparation can be formulated in combination with another agent, e.g., another therapeutic agent or an agent that stabilizes an oligonucleotide and/or dsRNA, e.g., a protein that complexes with oligonucleotide and/or dsRNA. Still other agents include chelating agents, e.g., EDTA (e.g., to remove divalent cations such as Mg²⁺), salts, RNAse inhibitors (e.g., a broad specificity RNAse inhibitor such as RNAsin) and so forth.

In some embodiments, the oligonucleotide and/or dsRNA preparation includes another dsRNA compound, e.g., a second dsRNA that can mediate RNAi with respect to a second gene, or with respect to the same gene. Still other preparation can include at least 3, 5, ten, twenty, fifty, or a hundred or more different siRNA species. Such dsRNAs can mediate RNAi with respect to a similar number of different genes.

In some embodiments, the oligonucleotide and/or dsRNA preparation includes at least a second therapeutic agent (e.g., an agent other than a RNA or a DNA). For example, a oligonucleotide and/or dsRNA composition for the treatment of a viral disease, e.g., HIV, might include a known antiviral agent (e.g., a protease inhibitor or reverse transcriptase inhibitor). In another example, a dsRNA composition for the treatment of a cancer might further comprise a chemotherapeutic agent.

Exemplary formulations which can be used for administering the oligonucleotide and/or dsRNA according to the present invention are discussed below.

Liposomes. A oligonucleotide and/or dsRNA preparation can be formulated for delivery in a membranous molecular assembly, e.g., a liposome or a micelle. As used herein, the term “liposome” refers to a vesicle composed of amphiphilic lipids arranged in at least one bilayer, e.g., one bilayer or a plurality of bilayers. Liposomes include unilamellar and multilamellar vesicles that have a membrane formed from a lipophilic material and an aqueous interior. The aqueous portion contains the oligonucleotide and/or dsRNA composition. The lipophilic material isolates the aqueous interior from an aqueous exterior, which typically does not include the oligonucleotide and/or dsRNA composition, although in some examples, it may. Liposomes are useful for the transfer and delivery of active ingredients to the site of action. Because the liposomal membrane is structurally similar to biological membranes, when liposomes are applied to a tissue, the liposomal bilayer fuses with bilayer of the cellular membranes. As the merging of the liposome and cell progresses, the internal aqueous contents that include the oligonucleotide and/or dsRNA are delivered into the cell where the dsRNA can specifically bind to a target RNA and can mediate RNAi. In some embodiments, the liposomes are also specifically targeted, e.g., to direct the oligonucleotide and/or dsRNA to particular cell types.

A liposome containing oligonucleotide and/or dsRNA can be prepared by a variety of methods. In one example, the lipid component of a liposome is dissolved in a detergent so that micelles are formed with the lipid component. For example, the lipid component can be an amphipathic cationic lipid or lipid conjugate. The detergent can have a high critical micelle concentration and may be nonionic. Exemplary detergents include cholate, CHAPS, octylglucoside, deoxycholate, and lauroyl sarcosine. The dsRNA preparation is then added to the micelles that include the lipid component. The cationic groups on the lipid interact with the siRNA and condense around the dsRNA to form a liposome. After condensation, the detergent is removed, e.g., by dialysis, to yield a liposomal preparation of oligonucleotide and/or dsRNA.

If necessary a carrier compound that assists in condensation can be added during the condensation reaction, e.g., by controlled addition. For example, the carrier compound can be a polymer other than a nucleic acid (e.g., spermine or spermidine). pH can also be adjusted to favor condensation.

Further description of methods for producing stable polynucleotide delivery vehicles, which incorporate a polynucleotide/cationic lipid complex as structural components of the delivery vehicle, are described in, e.g., WO 96/37194. Liposome formation can also include one or more aspects of exemplary methods described in Felgner, P. L. et al., Proc. Natl. Acad. Sci., USA 8:7413-7417, 1987; U.S. Pat. Nos. 4,897,355; 5,171,678; Bangham, et al. M. Mol. Biol. 23:238, 1965; Olson, et al. Biochim. Biophys. Acta 557:9, 1979; Szoka, et al. Proc. Natl. Acad. Sci. 75: 4194, 1978; Mayhew, et al. Biochim. Biophys. Acta 775:169, 1984; Kim, et al. Biochim. Biophys. Acta 728:339, 1983; and Fukunaga, et al. Endocrinol. 115:757, 1984, which are incorporated by reference in their entirety. Commonly used techniques for preparing lipid aggregates of appropriate size for use as delivery vehicles include sonication and freeze-thaw plus extrusion (see, e.g., Mayer, et al. Biochim. Biophys. Acta 858:161, 1986, which is incorporated by reference in its entirety). Microfluidization can be used when consistently small (50 to 200 nm) and relatively uniform aggregates are desired (Mayhew, et al. Biochim. Biophys. Acta 775:169, 1984, which is incorporated by reference in its entirety). These methods are readily adapted to packaging oligonucleotide and/or dsRNA preparations into liposomes.

Liposomes that are pH-sensitive or negatively-charged entrap nucleic acid molecules rather than complex with them. Since both the nucleic acid molecules and the lipid are similarly charged, repulsion rather than complex formation occurs. Nevertheless, some nucleic acid molecules are entrapped within the aqueous interior of these liposomes. pH-sensitive liposomes have been used to deliver DNA encoding the thymidine kinase gene to cell monolayers in culture. Expression of the exogenous gene was detected in the target cells (Zhou et al., Journal of Controlled Release, 19, (1992) 269-274, which is incorporated by reference in its entirety).

One major type of liposomal composition includes phospholipids other than naturally-derived phosphatidylcholine. Neutral liposome compositions, for example, can be formed from dimyristoyl phosphatidylcholine (DMPC) or dipalmitoyl phosphatidylcholine (DPPC). Anionic liposome compositions generally are formed from dimyristoyl phosphatidylglycerol, while anionic fusogenic liposomes are formed primarily from dioleoyl phosphatidylethanolamine (DOPE). Another type of liposomal composition is formed from phosphatidylcholine (PC) such as, for example, soybean PC, and egg PC. Another type is formed from mixtures of phospholipid and/or phosphatidylcholine and/or cholesterol.

Examples of other methods to introduce liposomes into cells in vitro and include U.S. Pat. Nos. 5,283,185; 5,171,678; WO 94/00569; WO 93/24640; WO 91/16024; Felgner, J. Biol. Chem. 269:2550, 1994; Nabel, Proc. Natl. Acad. Sci. 90:11307, 1993; Nabel, Human Gene Ther. 3:649, 1992; Gershon, Biochem. 32:7143, 1993; and Strauss EMBO J. 11:417, 1992.

In some embodiments, cationic liposomes are used. Cationic liposomes possess the advantage of being able to fuse to the cell membrane. Non-cationic liposomes, although not able to fuse as efficiently with the plasma membrane, are taken up by macrophages in vivo and can be used to deliver siRNAs to macrophages.

Further advantages of liposomes include: liposomes obtained from natural phospholipids are biocompatible and biodegradable; liposomes can incorporate a wide range of water and lipid soluble drugs; liposomes can protect encapsulated siRNAs in their internal compartments from metabolism and degradation (Rosoff, in “Pharmaceutical Dosage Forms,” Lieberman, Rieger and Banker (Eds.), 1988, volume 1, p. 245). Important considerations in the preparation of liposome formulations are the lipid surface charge, vesicle size and the aqueous volume of the liposomes.

A positively charged synthetic cationic lipid, N-[1-(2,3-dioleyloxy)propyl]-N,N,N-trimethylammonium chloride (DOTMA) can be used to form small liposomes that interact spontaneously with nucleic acid to form lipid-nucleic acid complexes which are capable of fusing with the negatively charged lipids of the cell membranes of tissue culture cells, resulting in delivery of siRNA (see, e.g., Felgner, P. L. et al., Proc. Natl. Acad. Sci., USA 8:7413-7417, 1987 and U.S. Pat. No. 4,897,355 for a description of DOTMA and its use with DNA, which are incorporated by reference in their entirety).

A DOTMA analogue, 1,2-bis(oleoyloxy)-3-(trimethylammonia)propane (DOTAP) can be used in combination with a phospholipid to form DNA-complexing vesicles. Lipofectin™ Bethesda Research Laboratories, Gaithersburg, Md.) is an effective agent for the delivery of highly anionic nucleic acids into living tissue culture cells that comprise positively charged DOTMA liposomes which interact spontaneously with negatively charged polynucleotides to form complexes. When enough positively charged liposomes are used, the net charge on the resulting complexes is also positive. Positively charged complexes prepared in this way spontaneously attach to negatively charged cell surfaces, fuse with the plasma membrane, and efficiently deliver functional nucleic acids into, for example, tissue culture cells. Another commercially available cationic lipid, 1,2-bis(oleoyloxy)-3,3-(trimethylammonia)propane (“DOTAP”) (Boehringer Mannheim, Indianapolis, Indiana) differs from DOTMA in that the oleoyl moieties are linked by ester, rather than ether linkages.

Other reported cationic lipid compounds include those that have been conjugated to a variety of moieties including, for example, carboxyspermine which has been conjugated to one of two types of lipids and includes compounds such as 5-carboxyspermylglycine dioctaoleoylamide (“DOGS”) (Transfectam™, Promega, Madison, Wisconsin) and dipalmitoylphosphatidylethanolamine 5-carboxyspermyl-amide (“DPPES”) (see, e.g., U.S. Pat. No. 5,171,678).

Another cationic lipid conjugate includes derivatization of the lipid with cholesterol (“DC-Chol”) which has been formulated into liposomes in combination with DOPE (See, Gao, X. and Huang, L., Biochim. Biophys. Res. Commun. 179:280, 1991). Lipopolylysine, made by conjugating polylysine to DOPE, has been reported to be effective for transfection in the presence of serum (Zhou, X. et al., Biochim. Biophys. Acta 1065:8, 1991, which is incorporated by reference in its entirety). For certain cell lines, these liposomes containing conjugated cationic lipids, are said to exhibit lower toxicity and provide more efficient transfection than the DOTMA-containing compositions. Other commercially available cationic lipid products include DMRIE and DMRIE-HP (Vical, La Jolla, California) and Lipofectamine (DOSPA) (Life Technology, Inc., Gaithersburg, Maryland). Other cationic lipids suitable for the delivery of oligonucleotides are described in WO 98/39359 and WO 96/37194.

Liposomal formulations are particularly suited for topical administration. Liposomes present several advantages over other formulations. Such advantages include reduced side effects related to high systemic absorption of the administered drug, increased accumulation of the administered drug at the desired target, and the ability to administer siRNA, into the skin. In some implementations, liposomes are used for delivering siRNA to epidermal cells and also to enhance the penetration of siRNA into dermal tissues, e.g., into skin. For example, the liposomes can be applied topically. Topical delivery of drugs formulated as liposomes to the skin has been documented (see, e.g., Weiner et al., Journal of Drug Targeting, 1992, vol. 2, 405-410 and du Plessis et al., Antiviral Research, 18, 1992, 259-265; Mannino, R. J. and Fould-Fogerite, S., Biotechniques 6:682-690, 1988; Itani, T. et al. Gene 56:267-276. 1987; Nicolau, C. et al. Meth. Enz. 149:157-176, 1987; Straubinger, R. M. and Papahadjopoulos, D. Meth. Enz. 101:512-527, 1983; Wang, C. Y. and Huang, L., Proc. Natl. Acad. Sci. USA 84:7851-7855, 1987, which are incorporated by reference in their entirety).

Non-ionic liposomal systems have also been examined to determine their utility in the delivery of drugs to the skin, in particular systems comprising non-ionic surfactant and cholesterol. Non-ionic liposomal formulations comprising Novasome I (glyceryl dilaurate/cholesterol/polyoxyethylene-10-stearyl ether) and Novasome II (glyceryl distearate/cholesterol/polyoxyethylene-10-stearyl ether) were used to deliver a drug into the dermis of mouse skin. Such formulations with dsRNA described herein are useful for treating a dermatological disorder.

Liposomes that include oligonucleotide and/or dsRNA described herein can be made highly deformable. Such deformability can enable the liposomes to penetrate through pore that are smaller than the average radius of the liposome. For example, transfersomes are a type of deformable liposomes. Transfersomes can be made by adding surface edge activators, usually surfactants, to a standard liposomal composition. Transfersomes that include oligonucleotide and/or dsRNA described herein can be delivered, for example, subcutaneously by infection in order to deliver dsRNA to keratinocytes in the skin. In order to cross intact mammalian skin, lipid vesicles must pass through a series of fine pores, each with a diameter less than 50 nm, under the influence of a suitable transdermal gradient. In addition, due to the lipid properties, these transfersomes can be self-optimizing (adaptive to the shape of pores, e.g., in the skin), self-repairing, and can frequently reach their targets without fragmenting, and often self-loading.

Other formulations amenable to the present invention are described in U.S. provisional application Ser. No. 61/018,616, filed Jan. 2, 2008; 61/018,611, filed Jan. 2, 2008; 61/039,748, filed Mar. 26, 2008; 61/047,087, filed Apr. 22, 2008 and 61/051,528, filed May 8, 2008. PCT application no PCT/US2007/080331, filed Oct. 3, 2007 also describes formulations that are amenable to the present invention.

Surfactants. The oligonucleotide and/or dsRNA compositions can include a surfactant. In some embodiments, the dsRNA is formulated as an emulsion that includes a surfactant. The most common way of classifying and ranking the properties of the many different types of surfactants, both natural and synthetic, is by the use of the hydrophile/lipophile balance (HLB). The nature of the hydrophilic group provides the most useful means for categorizing the different surfactants used in formulations (Rieger, in “Pharmaceutical Dosage Forms,” Marcel Dekker, Inc., New York, NY, 1988, p. 285).

If the surfactant molecule is not ionized, it is classified as a nonionic surfactant. Nonionic surfactants find wide application in pharmaceutical products and are usable over a wide range of pH values. In general, their HLB values range from 2 to about 18 depending on their structure. Nonionic surfactants include nonionic esters such as ethylene glycol esters, propylene glycol esters, glyceryl esters, polyglyceryl esters, sorbitan esters, sucrose esters, and ethoxylated esters. Nonionic alkanolamides and ethers such as fatty alcohol ethoxylates, propoxylated alcohols, and ethoxylated/propoxylated block polymers are also included in this class. The polyoxyethylene surfactants are the most popular members of the nonionic surfactant class.

If the surfactant molecule carries a negative charge when it is dissolved or dispersed in water, the surfactant is classified as anionic. Anionic surfactants include carboxylates such as soaps, acyl lactylates, acyl amides of amino acids, esters of sulfuric acid such as alkyl sulfates and ethoxylated alkyl sulfates, sulfonates such as alkyl benzene sulfonates, acyl isethionates, acyl taurates and sulfosuccinates, and phosphates. The most important members of the anionic surfactant class are the alkyl sulfates and the soaps.

If the surfactant molecule carries a positive charge when it is dissolved or dispersed in water, the surfactant is classified as cationic. Cationic surfactants include quaternary ammonium salts and ethoxylated amines. The quaternary ammonium salts are the most used members of this class.

If the surfactant molecule has the ability to carry either a positive or negative charge, the surfactant is classified as amphoteric. Amphoteric surfactants include acrylic acid derivatives, substituted alkylamides, N-alkylbetaines and phosphatides.

The use of surfactants in drug products, formulations and in emulsions has been reviewed (Rieger, in “Pharmaceutical Dosage Forms,” Marcel Dekker, Inc., New York, NY, 1988, p. 285).

Micelles and other Membranous Formulations. “Micelles” are defined herein as a particular type of molecular assembly in which amphipathic molecules are arranged in a spherical structure such that all the hydrophobic portions of the molecules are directed inward, leaving the hydrophilic portions in contact with the surrounding aqueous phase. The converse arrangement exists if the environment is hydrophobic.

A mixed micellar formulation suitable for delivery through transdermal membranes may be prepared by mixing an aqueous solution of the oligonucleotide and/or dsRNA composition, an alkali metal C₈ to C₂₂ alkyl sulphate, and a micelle forming compounds. Exemplary micelle forming compounds include lecithin, hyaluronic acid, pharmaceutically acceptable salts of hyaluronic acid, glycolic acid, lactic acid, chamomile extract, cucumber extract, oleic acid, linoleic acid, linolenic acid, monoolein, monooleates, monolaurates, borage oil, evening of primrose oil, menthol, trihydroxyl oxo cholanyl glycine and pharmaceutically acceptable salts thereof, glycerin, polyglycerin, lysine, polylysine, triolein, polyoxyethylene ethers and analogues thereof, polidocanol alkyl ethers and analogues thereof, chenodeoxycholate, deoxycholate, and mixtures thereof. The micelle forming compounds may be added at the same time or after addition of the alkali metal alkyl sulphate. Mixed micelles will form with substantially any kind of mixing of the ingredients but vigorous mixing in order to provide smaller size micelles.

In one method, a first micellar composition is prepared which contains the oligonucleotide and/or dsRNA composition and at least the alkali metal alkyl sulphate. The first micellar composition is then mixed with at least three micelle forming compounds to form a mixed micellar composition. In another method, the micellar composition is prepared by mixing the dsRNA composition, the alkali metal alkyl sulphate and at least one of the micelle forming compounds, followed by addition of the remaining micelle forming compounds, with vigorous mixing.

Phenol and/or m-cresol may be added to the mixed micellar composition to stabilize the formulation and protect against bacterial growth. Alternatively, phenol and/or m-cresol may be added with the micelle forming ingredients. An isotonic agent such as glycerin may also be added after formation of the mixed micellar composition.

For delivery of the micellar formulation as a spray, the formulation can be put into an aerosol dispenser and the dispenser is charged with a propellant. The propellant, which is under pressure, is in liquid form in the dispenser. The ratios of the ingredients are adjusted so that the aqueous and propellant phases become one, i.e., there is one phase. If there are two phases, it is necessary to shake the dispenser prior to dispensing a portion of the contents, e.g., through a metered valve. The dispensed dose of pharmaceutical agent is propelled from the metered valve in a fine spray.

Propellants may include hydrogen-containing chlorofluorocarbons, hydrogen-containing fluorocarbons, dimethyl ether and diethyl ether. In certain embodiments, HFA 134a (1,1,1,2 tetrafluoroethane) may be used.

The specific concentrations of the essential ingredients can be determined by relatively straightforward experimentation. For absorption through the oral cavities, it is often desirable to increase, e.g., at least double or triple, the dosage for through injection or administration through the gastrointestinal tract.

Particles. In some embodiments, dsRNA preparations can be incorporated into a particle, e.g., a microparticle. Microparticles can be produced by spray-drying, but may also be produced by other methods including lyophilization, evaporation, fluid bed drying, vacuum drying, or a combination of these techniques.

### Pharmaceutical Compositions

The oligonucleotide and/or dsRNA described herein can be formulated for pharmaceutical use. The present invention further relates to a pharmaceutical composition comprising the oligonucleotide and/or dsRNA described herein. Pharmaceutically acceptable compositions comprise a therapeutically-effective amount of one or more of the dsRNA molecules in any of the preceding embodiments, taken alone or formulated together with one or more pharmaceutically acceptable carriers (additives), excipient and/or diluents.

The pharmaceutical compositions may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally. Delivery using subcutaneous or intravenous methods can be particularly advantageous.

The phrase “therapeutically-effective amount” as used herein means that amount of a compound, material, or composition comprising a dsRNA molecule described herein which is effective for producing some desired therapeutic effect in at least a sub-population of cells in an animal at a reasonable benefit/risk ratio applicable to any medical treatment.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) lubricating agents, such as magnesium state, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; and (22) other non-toxic compatible substances employed in pharmaceutical formulations.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 0.1 percent to about ninety-nine percent of active ingredient, preferably from about 5 percent to about 70 percent, most preferably from about 10 percent to about 30 percent.

In certain embodiments, a formulation of the present invention comprises an excipient selected from the group consisting of cyclodextrins, celluloses, liposomes, micelle forming agents, e.g., bile acids, and polymeric carriers, e.g., polyesters and polyanhydrides; and a compound of the present invention. In certain embodiments, an aforementioned formulation renders orally bioavailable a compound of the present invention.

Methods of preparing these formulations or compositions include the step of bringing into association an oligonucleotide and/or dsRNA with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

The oligonucleotide and/or dsRNA described herein may be formulated for administration in any convenient way for use in human or veterinary medicine, by analogy with other pharmaceuticals.

The term “treatment” is intended to encompass therapy and cure. The patient receiving this treatment is any animal in need, including primates, in particular humans, and other mammals such as equines, cattle, swine and sheep; and poultry and pets in general.

The oligonucleotide and/or dsRNA described herein or a pharmaceutical composition comprising an oligonucleotide and/or dsRNA described herein can be administered to a subject using different routes of delivery. A composition that includes an oligonucleotide and/or dsRNA described herein described herein can be delivered to a subject by a variety of routes. Exemplary routes include: intravenous, subcutaneous, topical, rectal, anal, vaginal, nasal, pulmonary, ocular.

The oligonucleotide and/or dsRNA described herein may be administered in a number of ways depending upon whether local or systemic treatment is desired and upon the area to be treated. Administration may be topical (including ophthalmic, vaginal, rectal, intranasal, transdermal), oral or parenteral. Parenteral administration includes intravenous drip, subcutaneous, intraperitoneal or intramuscular injection, or intrathecal or intraventricular administration.

The route and site of administration may be chosen to enhance targeting. For example, to target muscle cells, intramuscular injection into the muscles of interest would be a logical choice. Lung cells might be targeted by administering the oligonucleotide and/or dsRNA described herein in aerosol form. The vascular endothelial cells could be targeted by coating a balloon catheter with the oligonucleotide and/or dsRNA described herein and mechanically introducing the oligonucleotide and/or dsRNA described herein.

In one aspect, provided herein is a method of administering an oligonucleotide and/or dsRNA described herein, to a subject (e.g., a human subject). In another aspect, the present invention relates to an oligonucleotide and/or dsRNA described herein for use in inhibiting expression of a target gene in a subject. The method or the medical use includes administering a unit dose of the oligonucleotide and/or dsRNA described herein. In some embodiments, the unit dose is less than 10 mg per kg of bodyweight, or less than 10, 5, 2, 1, 0.5, 0.1, 0.05, 0.01, 0.005, 0.001, 0.0005, 0.0001, 0.00005 or 0.00001 mg per kg of bodyweight, and less than 200 nmole of RNA agent (e.g., about 4.4×10¹⁶ copies) per kg of bodyweight, or less than 1500, 750, 300, 150, 75, 15, 7.5, 1.5, 0.75, 0.15, 0.075, 0.015, 0.0075, 0.0015, 0.00075, 0.00015 nmole of oligonucleotide and/or dsRNA described herein per kg of bodyweight.

The defined amount can be an amount effective to treat or prevent a disease or disorder, e.g., a disease or disorder associated with the target gene. The unit dose, for example, can be administered by injection (e.g., intravenous, subcutaneous or intramuscular), an inhaled dose, or a topical application. In some embodiments dosages may be less than 10, 5, 2, 1, or 0.1 mg/kg of body weight.

In some embodiments, the unit dose is administered less frequently than once a day, e.g., less than every 2, 4, 8 or 30 days. In another embodiment, the unit dose is not administered with a frequency (e.g., not a regular frequency). For example, the unit dose may be administered a single time.

In some embodiments, the effective dose is administered with other traditional therapeutic modalities.

In some embodiments, a subject is administered an initial dose and one or more maintenance doses. The maintenance dose or doses can be the same or lower than the initial dose, e.g., one-half less of the initial dose. A maintenance regimen can include treating the subject with a dose or doses ranging from 0.01 g to 15 mg/kg of body weight per day, e.g., 10, 1, 0.1, 0.01, 0.001, or 0.00001 mg per kg of bodyweight per day. The maintenance doses are, for example, administered no more than once every 2, 5, 10, or 30 days. Further, the treatment regimen may last for a period of time which will vary depending upon the nature of the particular disease, its severity and the overall condition of the patient. In certain embodiments the dosage may be delivered no more than once per day, e.g., no more than once per 24, 36, 48, or more hours, e.g., no more than once for every 5 or 8 days. Following treatment, the patient can be monitored for changes in his condition and for alleviation of the symptoms of the disease state. The dosage of the compound may either be increased in the event the patient does not respond significantly to current dosage levels, or the dose may be decreased if an alleviation of the symptoms of the disease state is observed, if the disease state has been ablated, or if undesired side-effects are observed.

The effective dose can be administered in a single dose or in two or more doses, as desired or considered appropriate under the specific circumstances. If desired to facilitate repeated or frequent infusions, implantation of a delivery device, e.g., a pump, semi-permanent stent (e.g., intravenous, intraperitoneal, intracisternal or intracapsular), or reservoir may be advisable.

In some embodiments, the composition includes a plurality of dsRNA molecule species. In another embodiment, the dsRNA molecule species has sequences that are non-overlapping and non-adjacent to another species with respect to a naturally occurring target sequence. In another embodiment, the plurality of dsRNA molecule species is specific for different naturally occurring target genes. In another embodiment, the dsRNA molecule is allele specific.

The oligonucleotide and/or dsRNA described herein can be administered to mammals, particularly large mammals such as nonhuman primates or humans in a number of ways.

In some embodiments, the administration of the oligonucleotide and/or dsRNA composition described herein is parenteral, e.g., intravenous (e.g., as a bolus or as a diffusible infusion), intradermal, intraperitoneal, intramuscular, intrathecal, intraventricular, intracranial, subcutaneous, transmucosal, buccal, sublingual, endoscopic, rectal, oral, vaginal, topical, pulmonary, intranasal, urethral or ocular. Administration can be provided by the subject or by another person, e.g., a health care provider. The medication can be provided in measured doses or in a dispenser which delivers a metered dose. Selected modes of delivery are discussed in more detail below.

The invention provides methods, compositions, and kits, for rectal administration or delivery of oligonucleotide and/or dsRNA composition described herein.

### Methods of Inhibiting Expression of a Target Gene

Aspects of the disclosure also relate to methods for inhibiting the expression of a target gene. The method comprises administering to the subject in an amount sufficient to inhibit expression of the target gene: (i) a double-stranded RNA described herein, where the wherein the first strand is complementary to a target gene; and/or (ii) an oligonucleotide described herein, wherein the oligonucleotide is complementary to a target gene.

The present disclosure further relates to a use of an oligonucleotide and/or dsRNA molecule described herein for inhibiting expression of a target gene in a target cell. The present disclosure further relates to a use of an oligonucleotide and/or dsRNA molecule described herein for inhibiting expression of a target gene in a target cell in vitro.

Another aspect the invention relates to a method of modulating the expression of a target gene in a cell, comprising administering to said cell an oligonucleotide and/or dsRNA molecule described herein. In some embodiments, the target gene is selected from the group consisting of Factor VII, Eg5, PCSK9, TPX2, apoB, SAA, TTR, RSV, PDGF beta gene, Erb-B gene, Src gene, CRK gene, GRB2 gene, RAS gene, MEKK gene, JNK gene, RAF gene, Erk1/2 gene, PCNA(p21) gene, MYB gene, JUN gene, FOS gene, BCL-2 gene, hepcidin, Activated Protein C, Cyclin D gene, VEGF gene, EGFR gene, Cyclin A gene, Cyclin E gene, WNT-1 gene, beta-catenin gene, c-MET gene, PKC gene, NFKB gene, STAT3 gene, survivin gene, Her2/Neu gene, topoisomerase I gene, topoisomerase II alpha gene, mutations in the p73 gene, mutations in the p21(WAF1/CIP1) gene, mutations in the p27(KIP1) gene, mutations in the PPM1D gene, mutations in the RAS gene, mutations in the caveolin I gene, mutations in the MIB I gene, mutations in the MTAI gene, mutations in the M68 gene, mutations in tumor suppressor genes, and mutations in the p53 tumor suppressor gene.

Some exemplary embodiments of the various aspects described herein can be described by the following:

Embodiment A: An oligonucleotide of the formula,

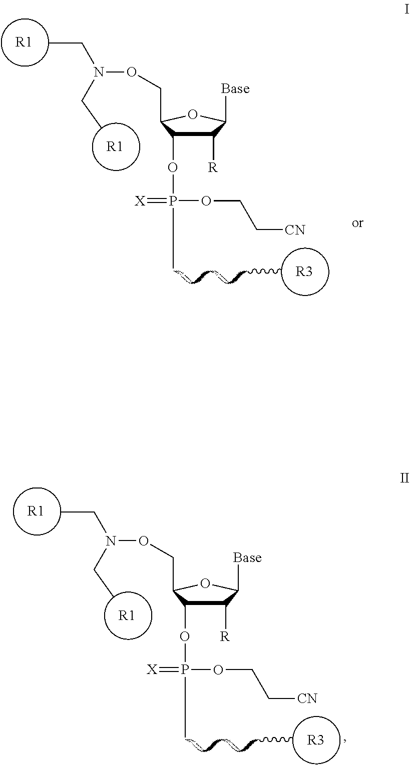

wherein: R is hydroxyl, protected hydroxyl, or a 2′-ribose modification (e.g., hydrogen, methoxy, halogen, fluoro, 2-methoxyethoxy, C₁₋₃₀ alkyl, or C₁₋₃₀ alkoxy such as n-hexadecyloxy), —O—C₄-30alkyl-ON(CH₂R¹)(CH₂R¹), or —O—C₄₋₃₀alkyl-ON(CH₂R¹)(CH₂R²); each R¹ and R² is independently a targeting ligand (e.g., GalNac), a pharmacokinetics modifier, C₁₋₃₀ alkyl, C₁₋₃₀ alkenyl, or C₁₋₃₀alkynyl, each optionally substituted with one or more aldehyde (—C(O)H), carboxylic acid (—COOH), C₁₋₁₀acyl (i.e., —C(O)C₁₋₁₀alkyl), hydroxyl, halogen, cyano, nitro, azido, thiol (i.e., —SH), amino, C₁₋₁₀ alkoxy, C₁₋₁₀ alkylthio, C₁₋₁₀alkylamino, di(C₁₋₁₀alkyl)amino, C₁₋₁₀-alkylcarboxylate (i.e., —C(O)OC₁₋₁₀alkyl), N—(C₁₋₁₀alkyl)amide (i.e., —C(O)NH(C₁₋₁₀alkyl)), N,N-di(C₁₋₁₀alkyl)amide (i.e., —C(O)N(C₁₋₁₀alkyl)₂), amino(C₁₋₁₀)acyl (i.e., —N(H)C(O)(C₁₋₁₀alkyl)), N—(C₁₋₁₀alkyl)amino(C₁₋₁₀)acyl(i.e., —N(C₁₋₁₀alkyl)C(O)(C₁₋₁₀alkyl)), keto (i.e., ═O) or thia (i.e., ═S); Base is a natural (A, C, G, T, or U) or modified nucleobase; X is O or S; and R3 is a 3′-oligonuclotide capping group (e.g., an inverted nucleotide or an inverted abasic nucleotide), a ligand, a linker covalently bonded to one or more ligands (e.g., N-acetylgalactosamine (GalNac)), a solid support, a linker covalently bonded (e.g., —C(O)CH₂CH₂C(O)—) to a solid support, hydrogen or OH, and wherein the oligonucleotide comprises 10-50 independently and optionally modified ribonucleotides (e.g., 2′-modified nucleotides with an R group as defined above, such as independently 2′-methoxy, 2′-deoxy, 2′-hexadecyloxy, or 2′-deoxy-2′-fluororibonucleotides)).

Embodiment B: An oligonucleotide of the formula,

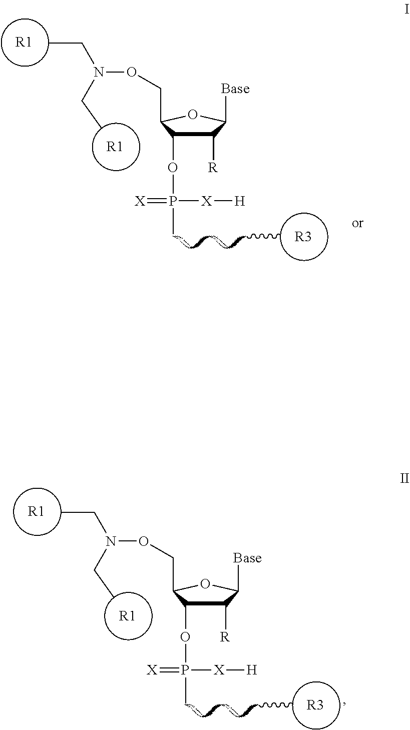

wherein: R is hydroxyl, protected hydroxyl, or a 2′-ribose modification (e.g., hydrogen, methoxy, halogen, fluoro, 2-methoxyethoxy, C₁₋₃₀ alkyl, or C₁₋₃₀ alkoxy such as n-hexadecyloxy), —O—C₄-30alkyl-ON(CH₂R¹)(CH₂R¹), or —O—C₄₋₃₀alkyl-ON(CH₂R¹)(CH₂R²); each R¹ and R² is independently a targeting ligand (e.g., GalNac), a pharmacokinetics modifier, C₁₋₃₀ alkyl, C₁₋₃₀ alkenyl, or C₁₋₃₀alkynyl, each optionally substituted with one or more aldehyde (—C(O)H), carboxylic acid (—COOH), C₁₋₁₀acyl (i.e., —C(O)C₁₋₁₀alkyl), hydroxyl, halogen, cyano, nitro, azido, thiol (i.e., —SH), amino, C₁₋₁₀ alkoxy, C₁₋₁₀ alkylthio, C₁₋₁₀alkylamino, di(C₁₋₁₀alkyl)amino, C₁₋₁₀-alkylcarboxylate (i.e., —C(O)OC₁₋₁₀alkyl), N—(C₁₋₁₀alkyl)amide (i.e., —C(O)NH(C₁₋₁₀alkyl)), N,N-di(C₁₋₁₀alkyl)amide (i.e., —C(O)N(C₁₋₁₀alkyl)₂), amino(C₁₋₁₀)acyl (i.e., —N(H)C(O)(C₁₋₁₀alkyl)), N—(C₁₋₁₀alkyl)amino(C₁₋₁₀)acyl(i.e., —N(C₁₋₁₀alkyl)C(O)(C₁₋₁₀alkyl)), keto (i.e., ═O) or thia (i.e., ═S); Base is a natural (A, C, G, T, or U) or modified nucleobase; each X is independently O or S (e.g., one is O and one is S or both are O); and R³ is a 3′-oligonuclotide capping group (e.g., an inverted nucleotide or an inverted abasic nucleotide), a ligand, a linker covalently bonded to one or more ligands (e.g., N-acetylgalactosamine (GalNac)), a solid support, a linker covalently bonded (e.g., —C(O)CH₂CH₂C(O)—) to a solid support, hydrogen or OH, and wherein the oligonucleotide comprises 10-50 independently and optionally modified ribonucleotides (e.g., 2′-modified nucleotides with an R group as defined above, such as independently 2′-methoxy, 2′-deoxy, 2′-hexadecyloxy, or 2′-deoxy-2′-fluororibonucleotides)).

Embodiment C: The oligonucleotide of Embodiment A or B, wherein R is hydrogen, methoxy, halogen, fluoro, 2-methoxyethoxy, C₁₋₃₀ alkyl, or C₁₋₃₀ alkoxy (such as n-hexadecyloxy).

Embodiment D: The oligonucleotide of any one of Embodiments A-C, wherein R is hydrogen, methoxy, fluoro, or 2-methoxyethoxy.

Embodiment E: The oligonucleotide of any one of Embodiments A-D, wherein R is hydrogen.

Embodiment F: The oligonucleotide of any one of Embodiments A-D, wherein R is methoxy.

Embodiment G: The oligonucleotide of any one of Embodiments A-D, wherein R is fluoro.

Embodiment H: The oligonucleotide of any one of Embodiments A-D, wherein R is methoxyethoxy.

Embodiment I: The oligonucleotide of any one of Embodiments A-H, wherein R¹ is a targeting ligand and R² is a pharmacokinetic modifier.

Embodiment JA double-stranded RNA comprising 20-100 nucleotides, wherein: the first strand is according to any one of Embodiments A-I, and the second strand optionally comprises 1 or more modified nucleotides and is complementary to the first strand, wherein Hone or both strands has a 1-5 nucleotide overhang on its respective 5′-end or 3′-end, or only one strand has a 2 nucleotide overhang on its 5′-end or 3′-end, or only one strand has a 2 nucleotide overhand on its 3′-end.

Embodiment K: A method of reducing the expression of a target gene in a subject (e.g., human), comprising administering to the subject either: (i) a double-stranded RNA according to Embodiment J, wherein the first strand or the second strand is complementary to a target gene; or (ii) an oligonucleotide according to any one of Embodiments A-I, wherein the oligonucleotide is complementary to a target gene.

Embodiment L: A compound of the formula,

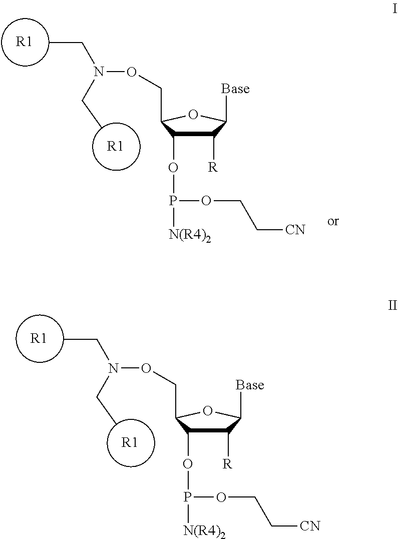

wherein: R is hydroxyl, protected hydroxyl, or a 2′-ribose modification (e.g., hydrogen, methoxy, halogen, fluoro, 2-methoxyethoxy, C₁₋₃₀ alkyl, or C₁₋₃₀ alkoxy such as n-hexadecyloxy), —O—C₄-30alkyl-ON(CH₂R¹)(CH₂R¹), or —O—C₄₋₃₀alkyl-ON(CH₂R¹)(CH₂R²); each R¹ and R² is independently a targeting ligand (e.g., GalNac), a pharmacokinetics modifier, C₁₋₃₀ alkyl, C₁₋₃₀ alkenyl, or C₁₋₃₀alkynyl, each optionally substituted with one or more aldehyde (—C(O)H), carboxylic acid (—COOH), C₁₋₁₀acyl (i.e., —C(O)C₁₋₁₀alkyl), hydroxyl, halogen, cyano, nitro, azido, thiol (i.e., —SH), amino, C₁₋₁₀ alkoxy, C₁₋₁₀ alkylthio, C₁₋₁₀alkylamino, di(C₁₋₁₀alkyl)amino, C₁₋₁₀-alkylcarboxylate (i.e., —C(O)OC₁₋₁₀alkyl), N—(C₁₋₁₀alkyl)amide (i.e., —C(O)NH(C₁₋₁₀alkyl)), N,N-di(C₁₋₁₀alkyl)amide (i.e., —C(O)N(C₁₋₁₀alkyl)₂), amino(C₁₋₁₀)acyl (i.e., —N(H)C(O)(C₁₋₁₀alkyl)), N—(C₁₋₁₀alkyl)amino(C₁₋₁₀)acyl(i.e., —N(C₁₋₁₀alkyl)C(O)(C₁₋₁₀alkyl)), keto (i.e., ═O) or thia (i.e., ═S); Base is a natural (A, C, G, T, or U) or modified nucleobase; each R⁴ is independently optionally substituted C₁₋₆alkyl (e.g., 2-propyl); or both R⁴ taken together with the nitrogen atom to which they are attached form an optionally substituted 3-8 membered heterocyclyl (e.g., 1-pyrrolidinyl), and wherein the substitutions at C2′ and C3′ of the ribose ring are optionally reversed (e.g., C3′ is substituted with R and C2′ is substituted with —OP(OCH₂CH₂CN)(NR4)₂

Embodiment M: The compound of Embodiment L, wherein R is hydrogen, methoxy, halogen, fluoro, 2-methoxyethoxy, C₁₋₃₀ alkyl, or C₁₋₃₀ alkoxy (such as n-hexadecyloxy).

Embodiment N: The compound of any one of Embodiments L-M, wherein R is hydrogen, methoxy, fluoro, or 2-methoxyethoxy.

Embodiment O: The compound of any one of Embodiments L-N, wherein R is hydrogen.

Embodiment P: The compound of any one of Embodiments L-N, wherein R is methoxy.

Embodiment Q: The compound of any one of Embodiments L-N, wherein R is fluoro.

Embodiment R: The compound of any one of Embodiments L-N, wherein R is methoxyethoxy.

Embodiment S: The compound of any one of Embodiments L-R, wherein R¹ is a targeting ligand and R² is a pharmacokinetic modifier.

### Some Selected Definitions

For convenience, certain terms employed herein, in the specification, examples and appended claims are collected herein. Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Further, unless otherwise required by context, singular terms shall include pluralities and plural terms shall include the singular.

Unless defined otherwise, all technical and scientific terms used herein have the same meaning as those commonly understood to one of ordinary skill in the art to which this invention pertains. Although any known methods, devices, and materials may be used in the practice or testing of the invention, the methods, devices, and materials in this regard are described herein.

Further, the practice of the present invention can employ, unless otherwise indicated, conventional techniques of molecular biology (including recombinant techniques), microbiology, cell biology, biochemistry, and immunology, which are within the skill of the art. Such techniques are explained fully in the literature, such as, “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Current Protocols in Molecular Biology” (F. M. Ausubel et al., eds., 1987, and periodic updates); “PCR: The Polymerase Chain Reaction”, (Mullis et al., ed., 1994); “A Practical Guide to Molecular Cloning” (Perbal Bernard V., 1988); “Phage Display: A Laboratory Manual” (Barbas et al., 2001).

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

As used herein the term “comprising” or “comprises” is used in reference to compositions, methods, and respective component(s) thereof, that are essential to the invention, yet open to the inclusion of unspecified elements, whether essential or not.

The singular terms “a,” “an,” and “the” include plural referents unless context clearly indicates otherwise. Similarly, the word “or” is intended to include “and” unless the context clearly indicates otherwise. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation.

As used herein, the term “alkyl” refers to an aliphatic hydrocarbon group which can be straight or branched having 1 to about 60 carbon atoms in the chain, and which preferably have about 6 to about 50 carbons in the chain. “Lower alkyl” refers to an alkyl group having 1 to about 8 carbon atoms. “Higher alkyl” refers to an alkyl group having about 10 to about 20 carbon atoms. The alkyl group can be optionally substituted with one or more alkyl group substituents which can be the same or different, where “alkyl group substituent” includes halo, amino, aryl, hydroxyl, alkoxy, aryloxy, alkyloxy, alkylthio, arylthio, aralkyloxy, aralkylthio, carboxy, alkoxycarbonyl, oxo and cycloalkyl. “Branched” refers to an alkyl group in which a lower alkyl group, such as methyl, ethyl or propyl, is attached to a linear alkyl chain. Exemplary alkyl groups include methyl, ethyl, propyl, i-propyl, n-butyl, t-butyl, n-pentyl, hexyl, heptyl, octyl, decyl, dodecyl, tridecyl, tetradecyl, pentadecyl and hexadecyl. Useful alkyl groups include branched or straight chain alkyl groups of 6 to 50 carbon, and also include the lower alkyl groups of 1 to about 4 carbons and the higher alkyl groups of about 12 to about 16 carbons.

A “heteroalkyl” group substitutes any one of the carbons of the alkyl group with a heteroatom having the appropriate number of hydrogen atoms attached (e.g., a CH₂ group to an NH group or an O group). The term “heteroalkyl” include optionally substituted alkyl, alkenyl and alkynyl radicals which have one or more skeletal chain atoms selected from an atom other than carbon, e.g., oxygen, nitrogen, sulfur, phosphorus, silicon, or combinations thereof. In certain embodiments, the heteroatom(s) is placed at any interior position of the heteroalkyl group. Examples include, but are not limited to, —CH₂—O—CH₃, —CH₂—CH₂—O—CH₃, —CH₂—NH—CH₃, —CH₂—CH₂—NH—CH₃, —CH₂—N(CH₃)—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CHO—CH₃, —Si(CH₃)₃, —CH₂—CH═N—OCH₃, and —CH═CH—N(CH₃)—CH₃. In some embodiments, up to two heteroatoms are consecutive, such as, by way of example, —CH₂—NH—OCH₃ and —CH₂—O—Si(CH₃)₃

As used herein, the term “alkenyl” refers to an alkyl group containing at least one carbon-carbon double bond. The alkenyl group can be optionally substituted with one or more “alkyl group substituents.” Exemplary alkenyl groups include vinyl, allyl, n-pentenyl, decenyl, dodecenyl, tetradecadienyl, heptadec-8-en-1-yl and heptadec-8,11-dien-1-yl.

As used herein, the term “alkynyl” refers to an alkyl group containing a carbon-carbon triple bond. The alkynyl group can be optionally substituted with one or more “alkyl group substituents.” Exemplary alkynyl groups include ethynyl, propargyl, n-pentynyl, decynyl and dodecynyl. Useful alkynyl groups include the lower alkynyl groups.

As used herein, the term “cycloalkyl” refers to a non-aromatic mono- or multicyclic ring system of about 3 to about 12 carbon atoms. The cycloalkyl group can be optionally partially unsaturated. The cycloalkyl group can be also optionally substituted with an aryl group substituent, oxo and/or alkylene. Representative monocyclic cycloalkyl rings include cyclopentyl, cyclohexyl and cycloheptyl. Useful multicyclic cycloalkyl rings include adamantyl, octahydronaphthyl, decalin, camphor, camphane, and noradamantyl.

“Heterocyclyl” refers to a nonaromatic 3-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively). Cₓheterocyclyl and Cₓ-C_{y}heterocyclyl are typically used where X and Y indicate the number of carbon atoms in the ring system. In some embodiments, 1, 2 or 3 hydrogen atoms of each ring can be substituted by a substituent. Exemplary heterocyclyl groups include, but are not limited to piperazinyl, pyrrolidinyl, dioxanyl, morpholinyl, tetrahydrofuranyl, piperidyl, 4-morpholyl, 4-piperazinyl, pyrrolidinyl, perhydropyrrolizinyl, 1,4-diazaperhydroepinyl, 1,3-dioxanyl, 1,4-dioxanyl and the like.

“Aryl” refers to an aromatic carbocyclic radical containing about 3 to about 13 carbon atoms. The aryl group can be optionally substituted with one or more aryl group substituents, which can be the same or different, where “aryl group substituent” includes alkyl, alkenyl, alkynyl, aryl, aralkyl, hydroxyl, alkoxy, aryloxy, aralkoxy, carboxy, aroyl, halo, nitro, trihalomethyl, cyano, alkoxycarbonyl, aryloxycarbonyl, aralkoxycarbonyl, acyloxy, acylamino, aroylamino, carbamoyl, alkylcarbamoyl, dialkylcarbamoyl, rylthio, alkylthio, alkylene and —NRR′, where R and R′ are each independently hydrogen, alkyl, aryl and aralkyl. Exemplary aryl groups include substituted or unsubstituted phenyl and substituted or unsubstituted naphthyl.

“Heteroaryl” refers to an aromatic 3-8 membered monocyclic, 8-12 membered fused bicyclic, or 11-14 membered fused tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively.

Exemplary aryl and heteroaryls include, but are not limited to, phenyl, pyridinyl, pyrimidinyl, furanyl, thienyl, imidazolyl, thiazolyl, pyrazolyl, pyridazinyl, pyrazinyl, triazinyl, tetrazolyl, indolyl, benzyl, naphthyl, anthracenyl, azulenyl, fluorenyl, indanyl, indenyl, naphthyl, tetrahydronaphthyl, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH carbazolyl, carbolinyl, chromanyl, chromenyl, cinnolinyl, decahydroquinolinyl, 2H,6H-1,5,2-dithiazinyl, dihydrofuro[2,3 b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl, and the like. In some embodiments, 1, 2, 3, or 4 hydrogen atoms of each ring can be substituted by a substituent.

As used herein, the term “halogen” or “halo” refers to an atom selected from fluorine, chlorine, bromine and iodine. The term “halogen radioisotope” or “halo isotope” refers to a radionuclide of an atom selected from fluorine, chlorine, bromine and iodine.

A “halogen-substituted moiety” or “halo-substituted moiety”, as an isolated group or part of a larger group, means an aliphatic, alicyclic, or aromatic moiety, as described herein, substituted by one or more “halo” atoms, as such terms are defined in this application.

The term “haloalkyl” as used herein refers to alkyl and alkoxy structures structure with at least one substituent of fluorine, chorine, bromine or iodine, or with combinations thereof. In embodiments, where more than one halogen is included in the group, the halogens are the same or they are different. The terms “fluoroalkyl” and “fluoroalkoxy” include haloalkyl and haloalkoxy groups, respectively, in which the halo is fluorine. Exemplary halo-substituted alkyl includes haloalkyl, dihaloalkyl, trihaloalkyl, perhaloalkyl and the like (e.g. halosubstituted (C₁-C₃)alkyl includes chloromethyl, dichloromethyl, difluoromethyl, trifluoromethyl (CF₃), perfluoroethyl, 2,2,2-trifluoroethyl, 2,2,2-trifluoro-1,1-dichloroethyl, and the like).

As used herein, the term “amino” means —NH₂. The term “alkylamino” means a nitrogen moiety having one straight or branched unsaturated aliphatic, cyclyl, or heterocyclyl radicals attached to the nitrogen, e.g., —NH(alkyl). The term “dialkylamino” means a nitrogen moiety having at two straight or branched unsaturated aliphatic, cyclyl, or heterocyclyl radicals attached to the nitrogen, e.g., —N(alkyl)(alkyl). The term “alkylamino” includes “alkenylamino,” “alkynylamino,” “cyclylamino,” and “heterocyclylamino.” The term “arylamino” means a nitrogen moiety having at least one aryl radical attached to the nitrogen. For example, -NHaryl, and —N(aryl)₂. The term “heteroarylamino” means a nitrogen moiety having at least one heteroaryl radical attached to the nitrogen. For example -NHheteroaryl, and —N(heteroaryl)₂. Optionally, two substituents together with the nitrogen can also form a ring. Unless indicated otherwise, the compounds described herein containing amino moieties can include protected derivatives thereof. Suitable protecting groups for amino moieties include acetyl, tertbutoxycarbonyl, benzyloxycarbonyl, and the like. Exemplary alkylamino includes, but is not limited to, NH(C₁-C₁₀alkyl), such as —NHCH₃, —NHCH₂CH₃, —NHCH₂CH₂CH₃, and —NHCH(CH₃)₂.

Exemplary dialkylamino includes, but is not limited to, —N(C₁-C₁₀alkyl)₂, such as N(CH₃)₂, —N(CH₂CH₃)₂, —N(CH₂CH₂CH₃)₂, and —N(CH(CH₃)₂)₂.

The term “aminoalkyl” means an alkyl, alkenyl, and alkynyl as defined above, except where one or more substituted or unsubstituted nitrogen atoms (—N—) are positioned between carbon atoms of the alkyl, alkenyl, or alkynyl. For example, an (C₂-C₆) aminoalkyl refers to a chain comprising between 2 and 6 carbons and one or more nitrogen atoms positioned between the carbon atoms.

The terms “hydroxyl” and “hydroxyl” mean the radical —OH.

The terms “alkoxyl” or “alkoxy” as used herein refers to an alkyl group, as defined above, having an oxygen radical attached thereto, and can be represented by one of —O-alkyl, —O— alkenyl, and —O-alkynyl. Aroxy can be represented by —O-aryl or O-heteroaryl, wherein aryl and heteroaryl are as defined herein. The alkoxy and aroxy groups can be substituted as described above for alkyl. Exemplary alkoxy groups include, but are not limited to O-methyl, O-ethyl, O-n-propyl, O-isopropyl, O-n-butyl, O-isobutyl, O-sec-butyl, O-tert-butyl, O-pentyl, O-hexyl, O-cyclopropyl, O-cyclobutyl, O-cyclopentyl, O-cyclohexyl and the like.

As used herein, the term “carbonyl” means the radical —C(O)—. It is noted that the carbonyl radical can be further substituted with a variety of substituents to form different carbonyl groups including acids, acid halides, amides, esters, ketones, and the like.

As used herein, the term “oxo” means double bonded oxygen, i.e., ═O.

The term “carboxy” means the radical —C(O)O—. It is noted that compounds described herein containing carboxy moieties can include protected derivatives thereof, i.e., where the oxygen is substituted with a protecting group. Suitable protecting groups for carboxy moieties include benzyl, tert-butyl, and the like. As used herein, a carboxy group includes -COGH, i.e., carboxyl group.

The term “ester” refers to a chemical moiety with formula —C(═O)OR, where R is selected from the group consisting of alkyl, cycloalkyl, aryl, heteroaryl and heterocycloalkyl.

The term “cyano” means the radical —CN.

The term “nitro” means the radical —NO₂.

The term, “heteroatom” refers to an atom that is not a carbon atom. Particular examples of heteroatoms include, but are not limited to nitrogen, oxygen, sulfur and halogens. A “heteroatom moiety” includes a moiety where the atom by which the moiety is attached is not a carbon. Examples of heteroatom moieties include —N═, —NR^{N}—, —N⁺(O⁻)═, —O—, —S— or —S(O)₂—, —OS(O)₂—, and —SS—, wherein R^{N} is H or a further substituent.

The terms “alkylthio” and “thioalkoxy” refer to an alkoxy group, as defined above, where the oxygen atom is replaced with a sulfur. In preferred embodiments, the “alkylthio” moiety is represented by one of —S-alkyl, —S-alkenyl, and —S-alkynyl. Representative alkylthio groups include methylthio, ethylthio, and the like. The term “alkylthio” also encompasses cycloalkyl groups, alkene and cycloalkene groups, and alkyne groups. “Arylthio” refers to aryl or heteroaryl groups.

The term “sulfinyl” means the radical —SO—. It is noted that the sulfinyl radical can be further substituted with a variety of substituents to form different sulfinyl groups including sulfinic acids, sulfinamides, sulfinyl esters, sulfoxides, and the like.

The term “sulfonyl” means the radical —SO₂—. It is noted that the sulfonyl radical can be further substituted with a variety of substituents to form different sulfonyl groups including sulfonic acids (—SO₃H), sulfonamides, sulfonate esters, sulfones, and the like.

The term “thiocarbonyl” means the radical —C(S)—. It is noted that the thiocarbonyl radical can be further substituted with a variety of substituents to form different thiocarbonyl groups including thioacids, thioamides, thioesters, thioketones, and the like.

“Acyl” refers to an alkyl-CO— group, wherein alkyl is as previously described. Exemplary acyl groups comprise alkyl of 1 to about 30 carbon atoms. Exemplary acyl groups also include acetyl, propanoyl, 2-methylpropanoyl, butanoyl and palmitoyl.

“Aroyl” means an aryl-CO— group, wherein aryl is as previously described.

Exemplary aroyl groups include benzoyl and 1- and 2-naphthoyl.

“Arylthio” refers to an aryl-S— group, wherein the aryl group is as previously described. Exemplary arylthio groups include phenylthio and naphthylthio.

“Aralkyl” refers to an aryl-alkyl- group, wherein aryl and alkyl are as previously described. Exemplary aralkyl groups include benzyl, phenylethyl and naphthylmethyl.

“Aralkyloxy” refers to an aralkyl-O— group, wherein the aralkyl group is as previously described. An exemplary aralkyloxy group is benzyloxy.

“Aralkylthio” refers to an aralkyl-S— group, wherein the aralkyl group is as previously described. An exemplary aralkylthio group is benzylthio.

“Alkoxycarbonyl” refers to an alkyl-O—CO— group. Exemplary alkoxycarbonyl groups include methoxycarbonyl, ethoxycarbonyl, butyloxycarbonyl, and t-butyloxycarbonyl.

“Aryloxycarbonyl” refers to an aryl-O—CO— group. Exemplary aryloxycarbonyl groups include phenoxy- and naphthoxy-carbonyl.

“Aralkoxycarbonyl” refers to an aralkyl-O—CO— group. An exemplary aralkoxycarbonyl group is benzyloxycarbonyl.

“Carbamoyl” refers to an H₂N—CO— group.

“Alkylcarbamoyl” refers to a R′RN—CO— group, wherein one of R and R′ is hydrogen and the other of R and R′ is alkyl as previously described.

“Dialkylcarbamoyl” refers to R′RN—CO— group, wherein each of R and R′ is independently alkyl as previously described.

“Acyloxy” refers to an acyl-O— group, wherein acyl is as previously described. “Acylamino” refers to an acyl-NH— group, wherein acyl is as previously described. “Aroylamino” refers to an aroyl-NH— group, wherein aroyl is as previously described.

The term “optionally substituted” means that the specified group or moiety is unsubstituted or is substituted with one or more (typically 1, 2, 3, 4, 5 or 6 substituents) independently selected from the group of substituents listed below in the definition for “substituents” or otherwise specified. The term “substituents” refers to a group “substituted” on a substituted group at any atom of the substituted group. Suitable substituents include, without limitation, halogen, hydroxyl, caboxy, oxo, nitro, haloalkyl, alkyl, alkenyl, alkynyl, alkaryl, aryl, heteroaryl, cyclyl, heterocyclyl, aralkyl, alkoxy, aryloxy, amino, acylamino, alkylcarbanoyl, arylcarbanoyl, aminoalkyl, alkoxycarbonyl, carboxy, hydroxylalkyl, alkanesulfonyl, arenesulfonyl, alkanesulfonamido, arenesulfonamido, aralkylsulfonamido, alkylcarbonyl, acyloxy, cyano or ureido. In some cases, two substituents, together with the carbons to which they are attached to can form a ring.

For example, any alkyl, alkenyl, cycloalkyl, heterocyclyl, heteroaryl or aryl is optionally substituted with 1, 2, 3, 4 or 5 groups selected from OH, CN, —SC(O)Ph, oxo (═O), SH, SO₂NH₂, SO₂(C₁-C₄)alkyl, SO₂NH(C₁-C₄)alkyl, halogen, carbonyl, thiol, cyano, NH₂, NH(C₁-C₄)alkyl, N[(C₁-C₄)alkyl]₂, C(O)NH₂, COOH, COOMe, acetyl, (C₁-C₈)alkyl, O(C₁-C₈)alkyl, O(C₁-C₈)haloalkyl, (C₂-C₈)alkenyl, (C₂-C₈)alkynyl, haloalkyl, thioalkyl, cyanomethylene, alkylaminyl, aryl, heteroaryl, substituted aryl, NH₂—C(O)-alkylene, NH(Me)-C(O)-alkylene, CH₂—C(O)-alkyl, C(O)— alkyl, alkylcarbonylaminyl, CH₂—[CH(OH)]ₘ—(CH₂)ₚ—OH, CH₂—[CH(OH)]ₘ—(CH₂)ₚ—NH₂ or CH₂-aryl-alkoxy; “m” and “p” are independently 1, 2, 3, 4, 5 or 6.

In some embodiments, an optionally substituted group is substituted with 1 substituent. In some other embodiments, an optionally substituted group is substituted with 2 independently selected substituents, which can be same or different. In some other embodiments, an optionally substituted group is substituted with 3 independently selected substituents, which can be same, different or any combination of same and different. In still some other embodiments, an optionally substituted group is substituted with 4 independently selected substituents, which can be same, different or any combination of same and different. In yet some other embodiments, an optionally substituted group is substituted with 5 independently selected substituents, which can be same, different or any combination of same and different.

An “isocyanato” group refers to a NCO group.

A “thiocyanato” group refers to a CNS group.

An “isothiocyanato” group refers to a NCS group.

“Alkoyloxy” refers to a RC(═O)O— group.

“Alkoyl” refers to a RC(═O)— group.

As used herein, the terms “dsRNA”, “siRNA”, and “iRNA agent” are used interchangeably to refer to agents that can mediate silencing of a target RNA, e.g., mRNA, e.g., a transcript of a gene that encodes a protein. For convenience, such mRNA is also referred to herein as mRNA to be silenced. Such a gene is also referred to as a target gene. In general, the RNA to be silenced is an endogenous gene, exogenous gene or a pathogen gene. In addition, RNAs other than mRNA, e.g., tRNAs, and viral RNAs, can also be targeted.

As used herein, the phrase “mediates RNAi” refers to the ability to silence, in a sequence specific manner, a target gene, e.g., mRNA. While not wishing to be bound by theory, it is believed that silencing uses the RNAi machinery or process and a guide RNA, e.g., antisense strand of a dsRNA, where the antisense strand is 21 to 23 nucleotides in length.

By “specifically hybridizable” and “complementary” is meant that a nucleic acid can form hydrogen bond(s) with another nucleic acid sequence by either traditional Watson-Crick or other non-traditional types. In reference to the nucleic molecules of the present invention, the binding free energy for a nucleic acid molecule with its complementary sequence is sufficient to allow the relevant function of the nucleic acid to proceed, e.g., RNAi activity. Determination of binding free energies for nucleic acid molecules is well known in the art (see, e.g., Turner et al, 1987, CSH Symp. Quant. Biol. LII pp. 123-133; Frier et al., 1986, Proc. Nat. Acad. Sci. USA 83:9373-9377; Turner et al., 1987, /. Am. Chem. Soc. 109:3783-3785). A percent complementarity indicates the percentage of contiguous residues in a nucleic acid molecule that can form hydrogen bonds (e.g., Watson-Crick base pairing) with a second nucleic acid sequence (e.g., 5, 6, 7, 8, 9,10 out of 10 being 50%, 60%, 70%, 80%, 90%, and 100% complementary). “Perfectly complementary” or 100% complementarity means that all the contiguous residues of a nucleic acid sequence will hydrogen bond with the same number of contiguous residues in a second nucleic acid sequence. Less than perfect complementarity refers to the situation in which some, but not all, nucleoside units of two strands can hydrogen bond with each other. “Substantial complementarity” refers to polynucleotide strands exhibiting 90% or greater complementarity, excluding regions of the polynucleotide strands, such as overhangs, that are selected so as to be noncomplementary. Specific binding requires a sufficient degree of complementarity to avoid non-specific binding of the oligomeric compound to non-target sequences under conditions in which specific binding is desired, i.e., under physiological conditions in the case of in vivo assays or therapeutic treatment, or in the case of in vitro assays, under conditions in which the assays are performed. The non-target sequences typically differ by at least 5 nucleotides.

The term “off-target” and the phrase “off-target effects” refer to any instance in which an effector molecule against a given target causes an unintended affect by interacting either directly or indirectly with another target sequence, a DNA sequence or a cellular protein or other moiety. For example, an “off-target effect” may occur when there is a simultaneous degradation of other transcripts due to partial homology or complementarity between that other transcript and the sense and/or antisense strand of an siRNA.

As used herein, the term “nucleoside” means a glycosylamine comprising a nucleobase and a sugar. Nucleosides includes, but are not limited to, naturally occurring nucleosides, abasic nucleosides, modified nucleosides, and nucleosides having mimetic bases and/or sugar groups.

As used herein, the term “nucleotide” refers to a glycosomine comprising a nucleobase and a sugar having a phosphate group covalently linked to the sugar. Nucleotides may be modified with any of a variety of substituents.

As used herein, the term “locked nucleic acid” or “LNA” or “locked nucleoside” or “locked nucleotide” refers to a nucleoside or nucleotide wherein the furanose portion of the nucleoside includes a bridge connecting two carbon atoms on the furanose ring, thereby forming a bicyclic ring system. Locked nucleic acids are also referred to as bicyclic nucleic acids (BNA).

As used herein, unless otherwise indicated, the term “methyleneoxy LNA” alone refers to β-D-methyleneoxy LNA.

As used herein, the term “MOE” refers to a 2′-O-methoxyethyl substituent.

As used herein, the term “gapmer” refers to a chimeric oligomeric compound comprising a central region (a “gap”) and a region on either side of the central region (the “wings”), wherein the gap comprises at least one modification that is different from that of each wing. Such modifications include nucleobase, monomeric linkage, and sugar modifications as well as the absence of modification (unmodified). Thus, in certain embodiments, the nucleotide linkages in each of the wings are different than the nucleotide linkages in the gap. In certain embodiments, each wing comprises nucleotides with high affinity modifications and the gap comprises nucleotides that do not comprise that modification. In certain embodiments the nucleotides in the gap and the nucleotides in the wings all comprise high affinity modifications, but the high affinity modifications in the gap are different than the high affinity modifications in the wings. In certain embodiments, the modifications in the wings are the same as one another. In certain embodiments, the modifications in the wings are different from each other. In certain embodiments, nucleotides in the gap are unmodified and nucleotides in the wings are modified. In certain embodiments, the modification(s) in each wing are the same. In certain embodiments, the modification(s) in one wing are different from the modification(s) in the other wing. In certain embodiments, oligomeric compounds are gapmers having 2′-deoxynucleotides in the gap and nucleotides with high-affinity modifications in the wing.

The term ‘BNA’ refers to bridged nucleic acid, and is often referred as constrained or inaccessible RNA. BNA can contain a 5-, 6-membered, or even a 7-membered bridged structure with a “fixed” C₃′-endo sugar puckering. The bridge is typically incorporated at the 2′-, 4′-position of the ribose to afford a 2′, 4′-BNA nucleotide (e.g., LNA, or ENA). Examples of BNA nucleotides include the following nucleosides:

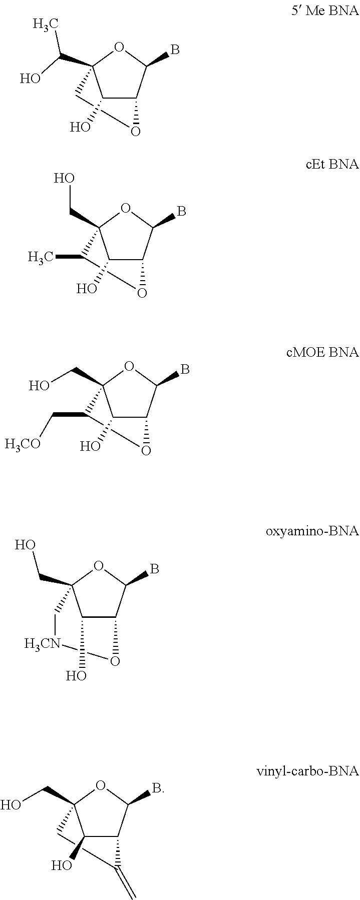

The term ‘ENA’ refers to ethylene-bridged nucleic acid, and is often referred as constrained or inaccessible RNA.

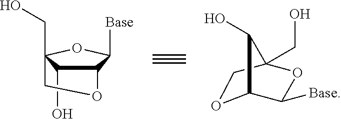

The “cleavage site” herein means the backbone linkage in the target gene or the sense strand that is cleaved by the RISC mechanism by utilizing the iRNA agent. And the target cleavage site region comprises at least one or at least two nucleotides on both side of the cleavage site. For the sense strand, the cleavage site is the backbone linkage in the sense strand that would get cleaved if the sense strand itself was the target to be cleaved by the RNAi mechanism. The cleavage site can be determined using methods known in the art, for example the 5′-RACE assay as detailed in Soutschek et al., Nature (2004) 432, 173-178, which is incorporated by reference in its entirety. As is well understood in the art, the cleavage site region for a conical double stranded RNAi agent comprising two 21-nucleotides long strands (wherein the strands form a double stranded region of 19 consecutive base pairs having 2-nucleotide single stranded overhangs at the 3′-ends), the cleavage site region corresponds to positions 9-12 from the 5′-end of the sense strand.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99% or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

As used herein, a “terminal region” of a strand refers to positions 1-4, e.g., positions 1, 2, 3, and 4, counting from the nearest end of the strand. For example, a 5′-terminal region refers to positions 1-4, e.g., positions 1, 2, 3 and 4 counting from the 5′-end of the strand. Similarly, a 3′-terminal region refers to positions 1-4, e.g., positions 1, 2, 3 and 4 counting from the 3′-end of the strand.

For example, a 5′-terminal region for the antisense strand is positions 1, 2, 3 and 4 counting from the 5′-end of the antisense strand. A preferred 5′-terminal region for the antisense strand is positions 1, 2 and 3 counting from the 5′-end of the antisense strand. A 3′-terminal region for the antisense strand can be positions 1, 2, 3, and 4 counting from the 3′-end of the strand. A preferred 3′-terminal region for the antisense strand is positions 1, 2 and 3 counting from the 3′-end of the antisense strand.

Similarly, a 5′-terminal region for the sense strand is positions 1, 2, 3 and 4 counting from the 5′-end of the sense strand. A preferred 5′-terminal region for the sense strand is positions 1, 2 and 3 counting from the 5′-end of the sense strand. A 3′-terminal region for the sense strand can be positions 1, 2, 3, and 4 counting from the 3′-end of the strand. A preferred 3′-terminal region for the sense strand is positions 1, 2 and 3 counting from the 3′-end of the sense strand.

As used herein, a “central region” of a strand refers to positions 5-17, e.g., positions 6-16, positions 6-15, positions 6-14, positions 6-13, positions 6-12, positions 7-15, positions 7-14, positions 7-13, positions, 7-12, positions 8-16, positions 8-15, positions 8-14, positions 8-13, positions 8-12, positions 9-16, positions 9-15, positions 9-14, positions 9-13, positions 9-12, positions 10-16, positions 10-15, positions 10-14, positions 10-13 or positions 10-12, counting from the 5′-end of the strand. For example, the central region of a strand means positions 5, 6, 7, 8, 9, 10, 11, 12, 13, 14, 15, 16 or 17 of the strand. A preferred central region for the sense strand is positions 6, 7, 8, 9, 10, 11, 12, 13, and 14, counting from the 5′-end of the sense strand. A more preferred central region for the sense strand is positions 7, 8, 9, 10, 11, 12 and 13, counting from the 5′-end of the sense strand. A preferred central region for the antisense strand is positions 9, 10, 11, 12, 13, 14, 15 16 and 17, counting from 5′-end of the antisense strand. A more preferred central region for the antisense strand is positions 10, 11, 12, 13, 14, 15 and 16, counting from 5′-end of the antisense strand.

As used herein, the term “in vitro” refers to events that occur in an artificial environment, e.g., in a test tube or reaction vessel, in cell culture, etc., rather than within an organism (e.g. animal or a plant). As used herein, the term “ex vivo” refers to cells which are removed from a living organism and cultured outside the organism (e.g., in a test tube). As used herein, the term “in vivo” refers to events that occur within an organism (e.g. animal, plant, and/or microbe).

As used herein, the term “subject” or “patient” refers to any organism to which a composition disclosed herein can be administered, e.g., for experimental, diagnostic, and/or therapeutic purposes. Typical subjects include animals (e.g., mammals such as mice, rats, rabbits, non-human primates, and humans) and/or plants. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. Patient or subject includes any subset of the foregoing, e.g., all of the above, but excluding one or more groups or species such as humans, primates or rodents. In certain embodiments of the aspects described herein, the subject is a mammal, e.g., a primate, e.g., a human. The terms, “patient” and “subject” are used interchangeably herein. A subject can be male or female.

Preferably, the subject is a mammal. The mammal can be a human, non-human primate, mouse, rat, dog, cat, horse, or cow, but are not limited to these examples. Mammals other than humans can be advantageously used as subjects that represent animal models of human diseases and disorders. In addition, compounds, compositions and methods described herein can be used to with domesticated animals and/or pets.

In some embodiments, the subject is human. In another embodiment, the subject is an experimental animal or animal substitute as a disease model. The term does not denote a particular age or sex. Thus, adult and newborn subjects, as well as fetuses, whether male or female, are intended to be covered. Examples of subjects include humans, dogs, cats, cows, goats, and mice. The term subject is further intended to include transgenic species. In some embodiments, the subject can be of European ancestry. In some embodiments, the subject can be of African American ancestry. In some embodiments, the subject can be of Asian ancestry.

In jurisdictions that forbid the patenting of methods that are practiced on the human body, the meaning of “administering” of a composition to a human subject shall be restricted to prescribing a controlled substance that a human subject will self-administer by any technique (e.g., orally, inhalation, topical application, injection, insertion, etc.). The broadest reasonable interpretation that is consistent with laws or regulations defining patentable subject matter is intended. In jurisdictions that do not forbid the patenting of methods that are practiced on the human body, the “administering” of compositions includes both methods practiced on the human body and also the foregoing activities.

As used herein, the term “parenteral administration,” refers to administration through injection or infusion. Parenteral administration includes, but is not limited to, subcutaneous administration, intravenous administration, or intramuscular administration.

As used herein, the term “subcutaneous administration” refers to administration just below the skin. “Intravenous administration” means administration into a vein.

As used herein, the term “dose” refers to a specified quantity of a pharmaceutical agent provided in a single administration. In certain embodiments, a dose may be administered in two or more boluses, tablets, or injections. For example, in certain embodiments, where subcutaneous administration is desired, the desired dose requires a volume not easily accommodated by a single injection. In such embodiments, two or more injections may be used to achieve the desired dose. In certain embodiments, a dose may be administered in two or more injections to minimize injection site reaction in an individual.

As used herein, the term “dosage unit” refers to a form in which a pharmaceutical agent is provided. In certain embodiments, a dosage unit is a vial comprising lyophilized antisense oligonucleotide. In certain embodiments, a dosage unit is a vial comprising reconstituted antisense oligonucleotide.

It should be understood that this disclosure is not limited to the particular methodology, protocols, and reagents, etc., provided herein and as such may vary. The terminology used herein is for the purpose of describing particular embodiments only, and is not intended to limit the scope of the present disclosure, which is defined solely by the claims. The invention is further illustrated by the following example, which should not be construed as further limiting.

### EXAMPLES

### Example 1: Aminooxy Click Chemistry (AOCC): A Versatile Bioconjugation Approach

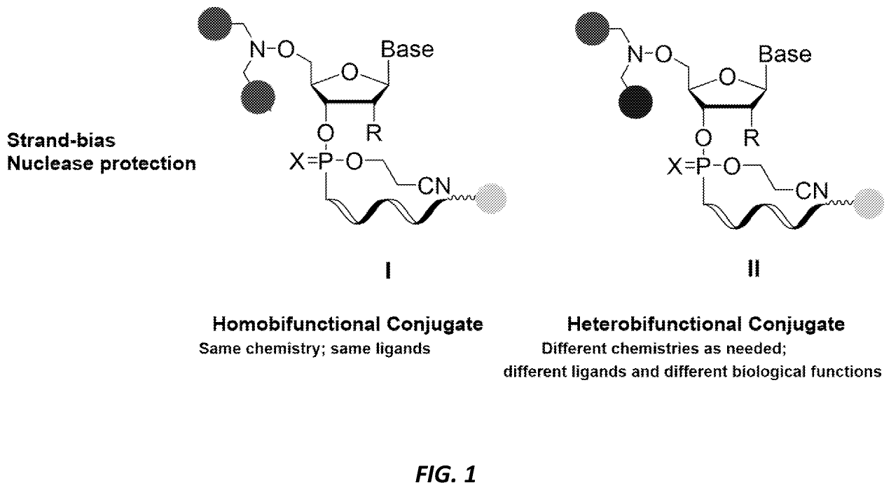

Conjugation chemistry has invigorated oligonucleotide therapeutics field as demonstrated by the clinical success of siRNA conjugated to GalNAc, the ligand for the asialoglycoprotein (ASGPR) hepatocyte-specific receptor. Aminooxy-functionalized (—O—NH₂) sugars and nucleosides have attracted interest as they can be easily derivatized through oxime ligation (Rodriguez, E. C.; Marcaurelle, L. A.; Bertozzi, C. R. J. Org. Chem. 1998, 63, 7134; Salo, H.; Virta, P.; Hakala, H.; Prakash, T. P.; Kawasaki, A. M.; Manoharan, M.; Lönnberg, H. Bioconjugate Chemistry 1999, 10, 815; Xie, J.; Peyrat, S. Synthesis 2012, 44, 1718; and Meyer, A.; Vasseur, J.-J.; Dumy, P.; Morvan, F. Eur. J. Org. Chem. 2017, 2017, 6931) and N-oxyamide functionalization (Noel, M.; Clement-Blanc, C.; Meyer, A.; Vasseur, J.-J.; Morvan, F. J. Org. Chem. 2019, 84, 14854). Compounds derived from O-amino carbohydrates and O-amino nucleosides have proven useful in various biological applications (Chen, N.; Xie, J. Molecules 2018, 23, 641/1 and Pifferi, C.; Daskhan, G. C.; Fiore, M.; Shiao, T. C.; Roy, R.; Renaudet, O. Chem. Rev. 2017, 117, 9839). In the present work, the inventors developed a simple “click type” amino-oxy-based chemistry that we call aminooxy click chemistry (AOCC) and used it to synthesize 2′-, 3′- and 5′-aminooxy nucleosides conjugated to bis-homo (I) and bis-hetero (II) ligands (FIG. 1) The bis-homo ligand conjugation leads to bivalent ligand presentation. The bis-hetero conjugation will allow placement of ligands with different chemical functions, for example, an aldehyde and an acid; and also ligands with different biological functions such as a targeting ligand and a pharmacokinetics modifier to the same —O—NH₂ linkage. In the context of RNA interference-based therapeutics, furthermore, we are in the process of testing whether these conjugates enhance better strand-bias for antisense strand incorporation into the RNA-induced silencing complex and improved metabolic stability from nucleases.

### BACKGROUND

Impact of various nucleoside modifications to modulate PK and delivery properties.
Limiting factors are PK modulation and tissue and cellular delivery.
GalNAc has been used as powerful tool through ASGPR to overcome such challenges.
Other targets achieved by lipophilic modifications (e.g., cholesterol).
Folate (Folate receptor α (FRα) came into focus as an anticancer¹ target) and integrins (transmembrane receptors) are other major target with persisting limitations.

In this study, inventors describe aminooxy functionality which can form a new class of oligonucleotide drugs.

While aminooxy group has been used in the oligo-nucleotide chemistry as an active group for conjugation of various group²⁻⁶ as permanent or reversible functional group⁷, mostly the oxime formation has been utilized for such ligation.⁶ When the oxime bond undergoes reductive amination, mostly the second alkyl groups used, were methyl (CH₃), ethyl, isopropyl etc.⁸⁻⁹ Interestingly, aminooxy chemistry has been used widely in sugar¹⁰⁻¹², aminooxy peptide¹³⁻¹⁸, and neutral nucleoside backbone chemistry¹⁹⁻²⁶ apart from conjugation chemistry which broadens its scope considerably. The inventors have used aminooxy group in four different chemistries to demonstrate its application in the field of oligonucleotide synthesis and named it as aminooxy click chemistry (AOCC) approach. The classifications are as follows:
1. Oxime type conjugation;
2. Mono substituted aminooxy (second ligand being simple CH₃ group);
   And more importantly
3. Bis-homo ligands conjugation. (same ligand, two times); and
4. Bis-hetero ligands conjugation. (different ligands, one after another).

To accomplish these targets, two different strategies were employed viz., (a) synthesis of monomers as amidites and incorporating those in oligonucleotide synthesis and (b) introducing the precursors of aminooxy groups into the oligo nucleotide (ON) strands and then functionalize those post-synthetically. A general representation of these routes are described below.

### Synthesis of Building Blocks (Monomer Amidites and Controlled Pore Glass CPG) in Solution Phase for Modifications at 3′-, 5′-, and Internal Positions of ONs

### Synthesis of Exemplary Amidites for 5′-End Modifications are Shown in Schemes 1 and 2

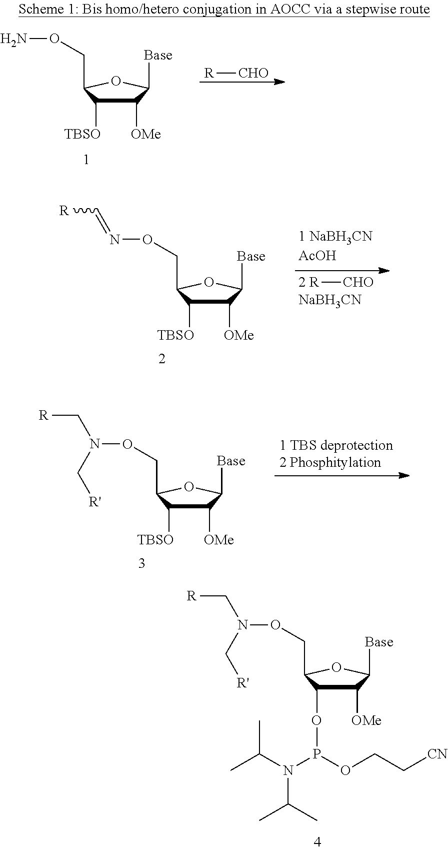

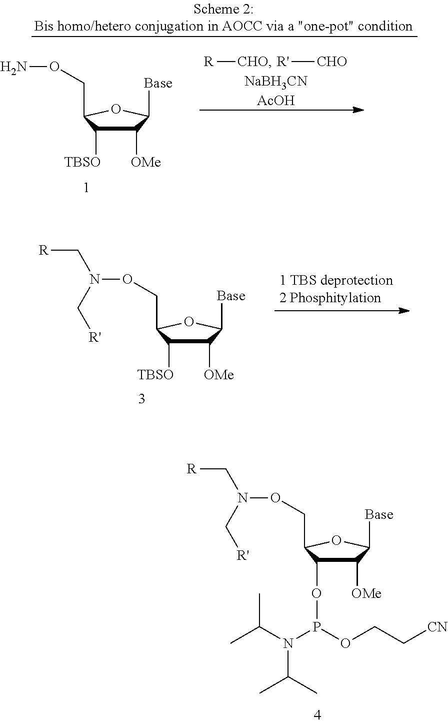

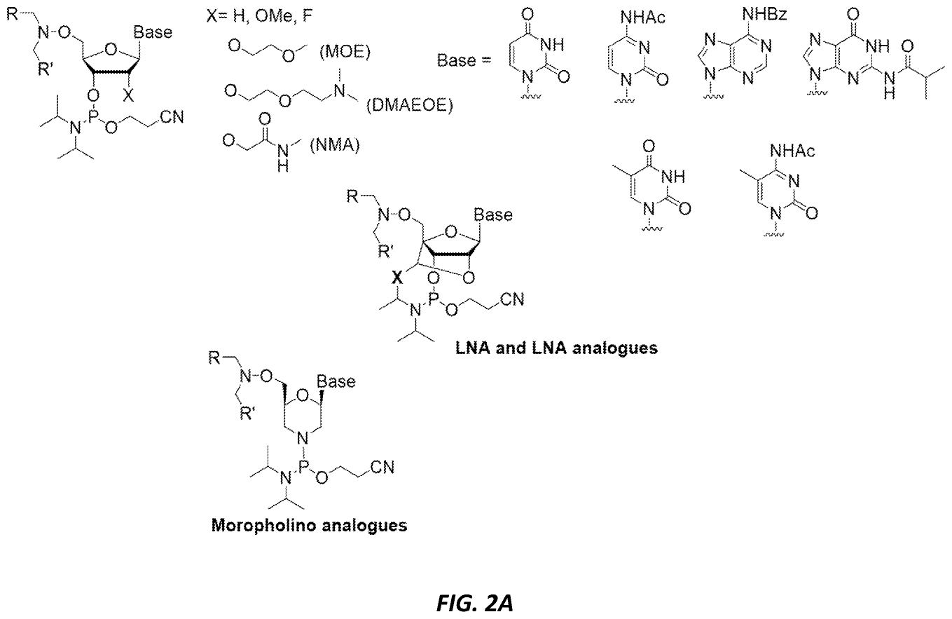

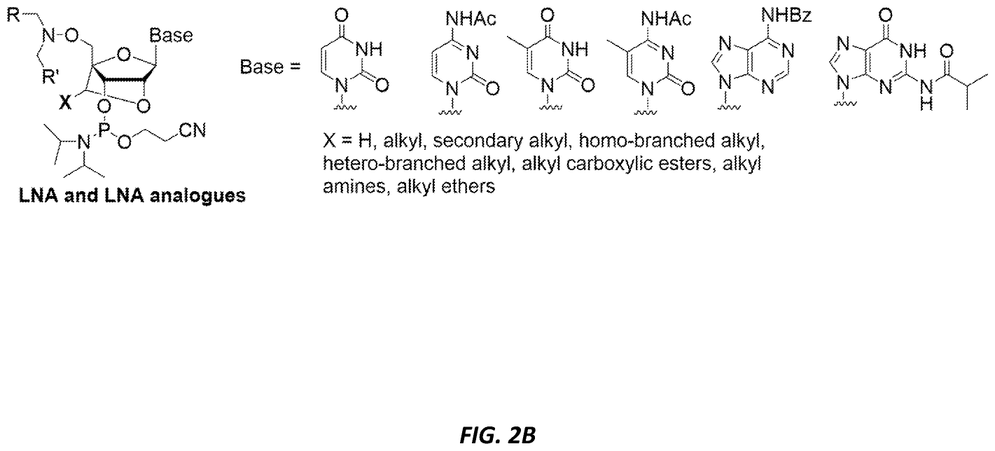

Some exemplary 5′-end aminooxy modified nucleoside scaffolds are shown in FIG. 2.

Synthesis of 2′- and 3′-modified amidites for conjugation at internal positions of oligonucleotides is shown in Scheme 3.

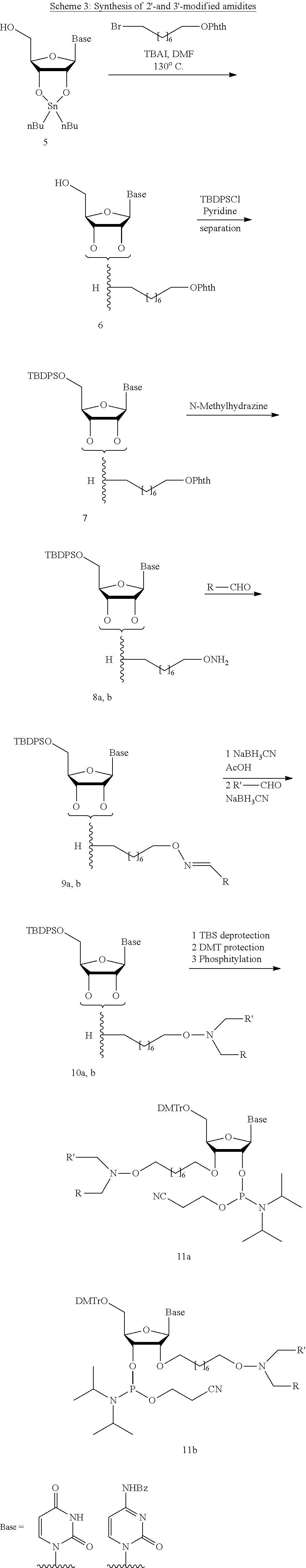

### Ligand Post-Synthesis Conjugation Plan on Solid Support

Monomers and 5′-end modifications for AOCC on solid support are shown in Scheme 4 and 5.

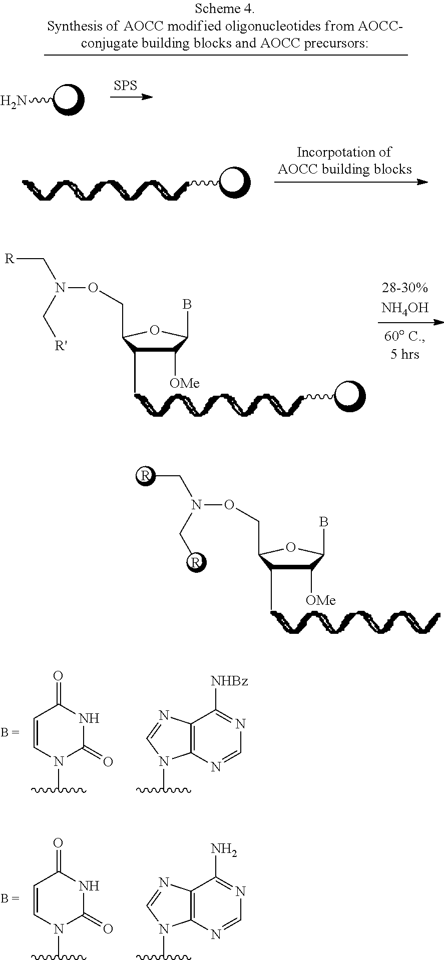

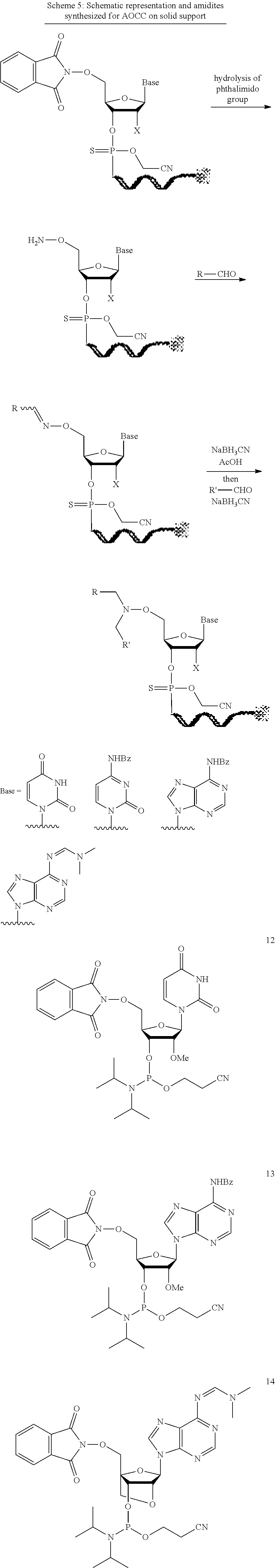

Synthesis of exemplary 2′- and 3′-AOCC modified amidites and CPGs for conjugation at internal and 3′-end conjugation of ONs on solid support are shown in Scheme 6.

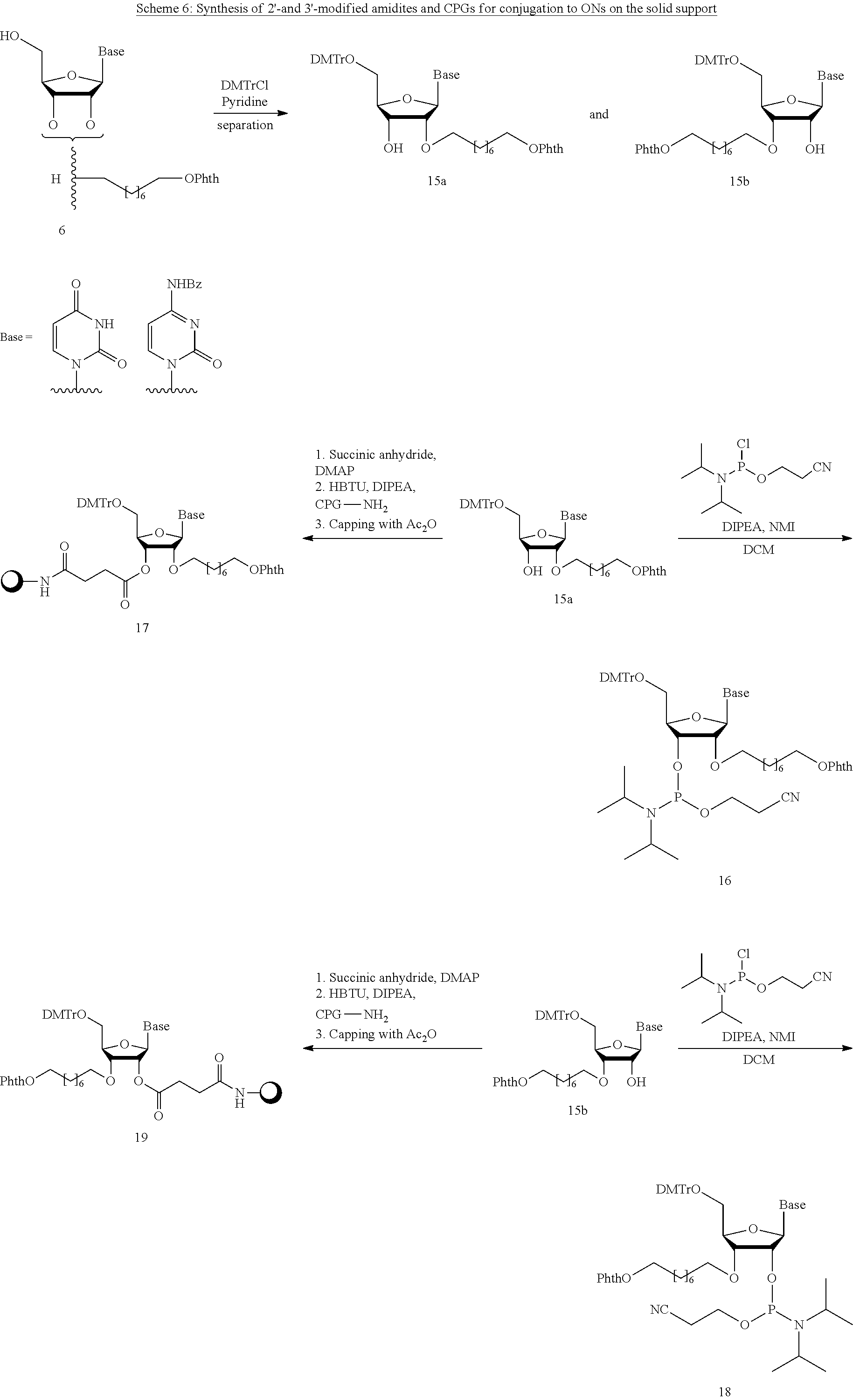

Some exemplary 2′- and 3′-aminooxy conjugated nucleoside building blocks are shown in FIGS. 3 (2′-analogues) and 4 (3′-analogues).

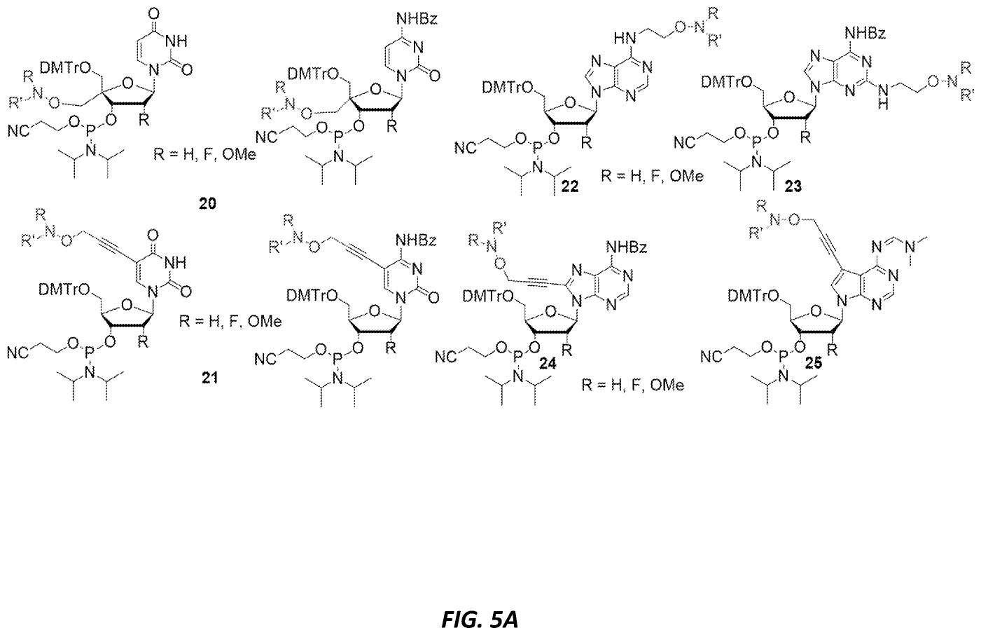

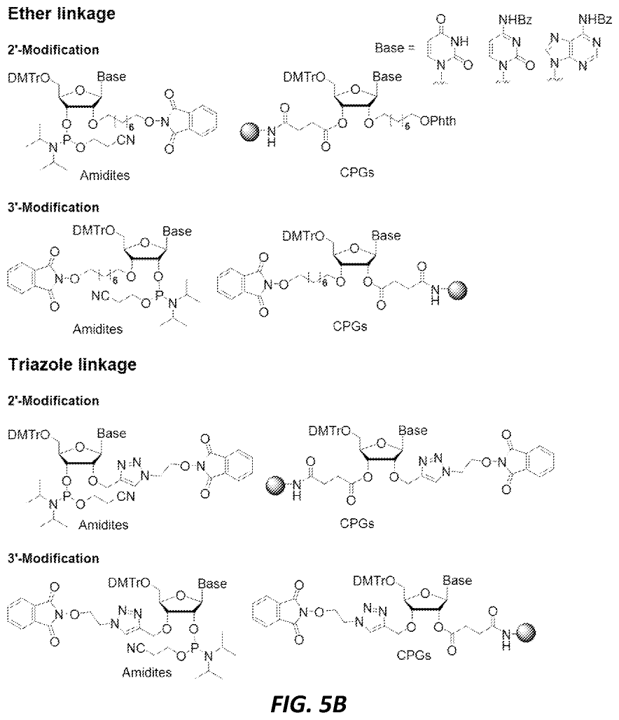

FIG. 5 shows some exemplary positions in nucleosides for AOCC: (1) Sugar modifications: 2′ (11b, 16, 17), 3′ (11a, 18. 19), 4′ (20) and 5′ (4) modifications; and (2) Base modifications: (i) C5 modification for pyrimidines and N4 modification for pyrimidines (21); and (ii) N2, N6, C8 and N7-deaza for purines (22-25).

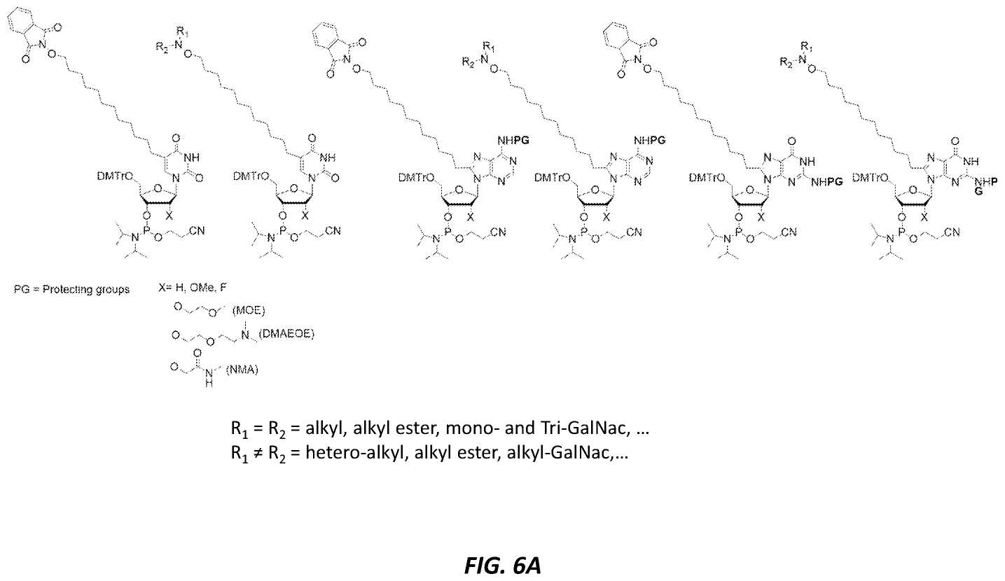

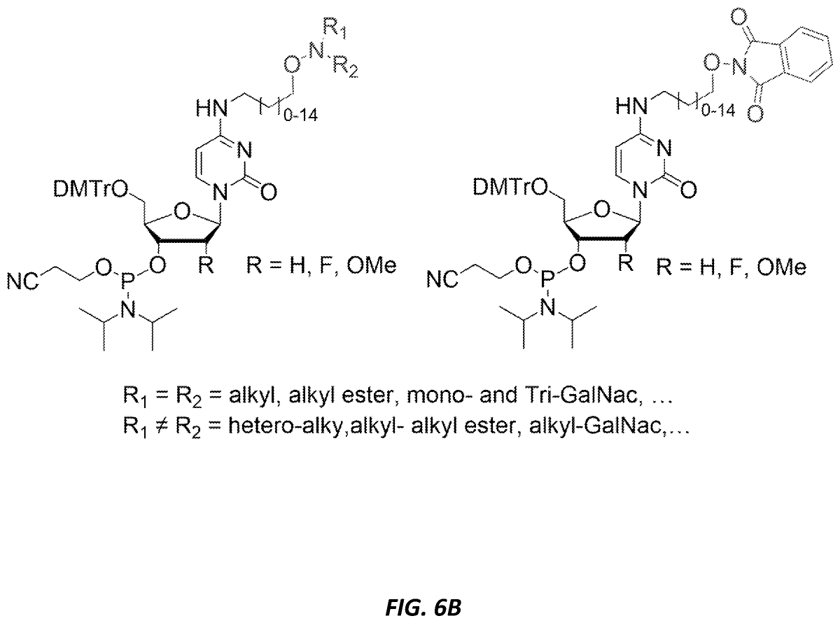

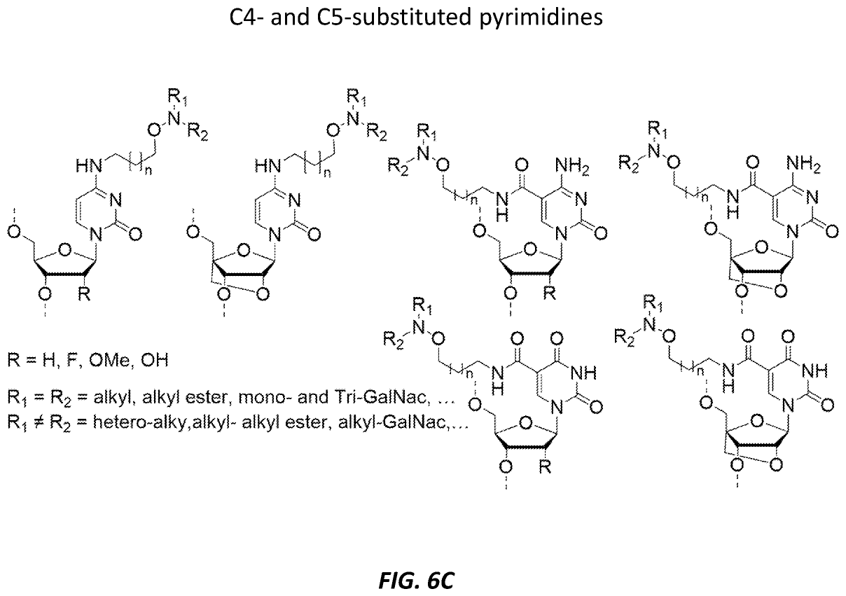

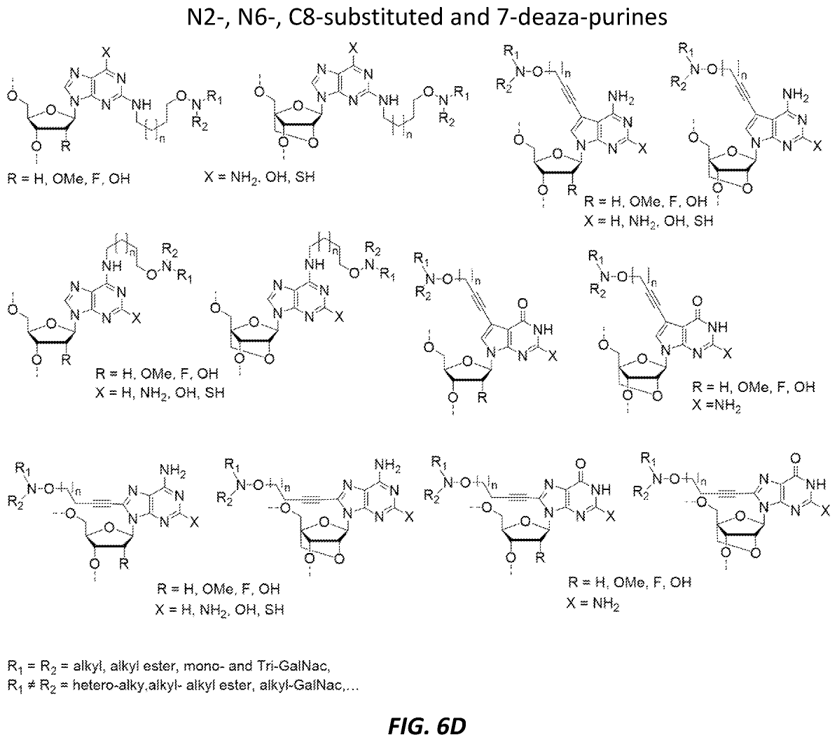

FIG. 6 depicts some exemplary monomers for AOCC at the nucleobase.

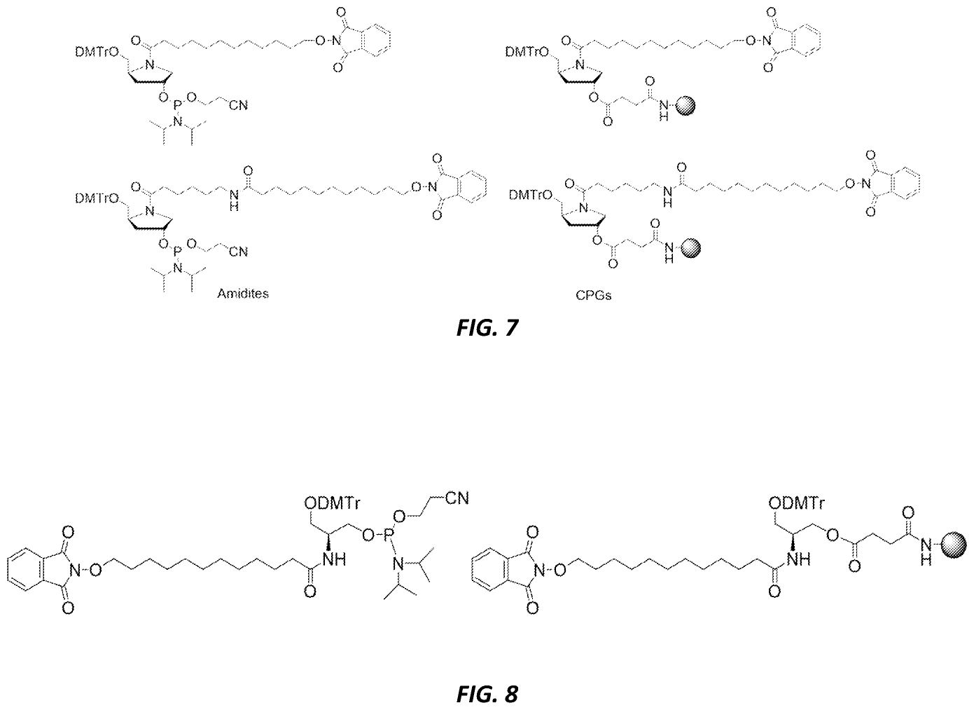

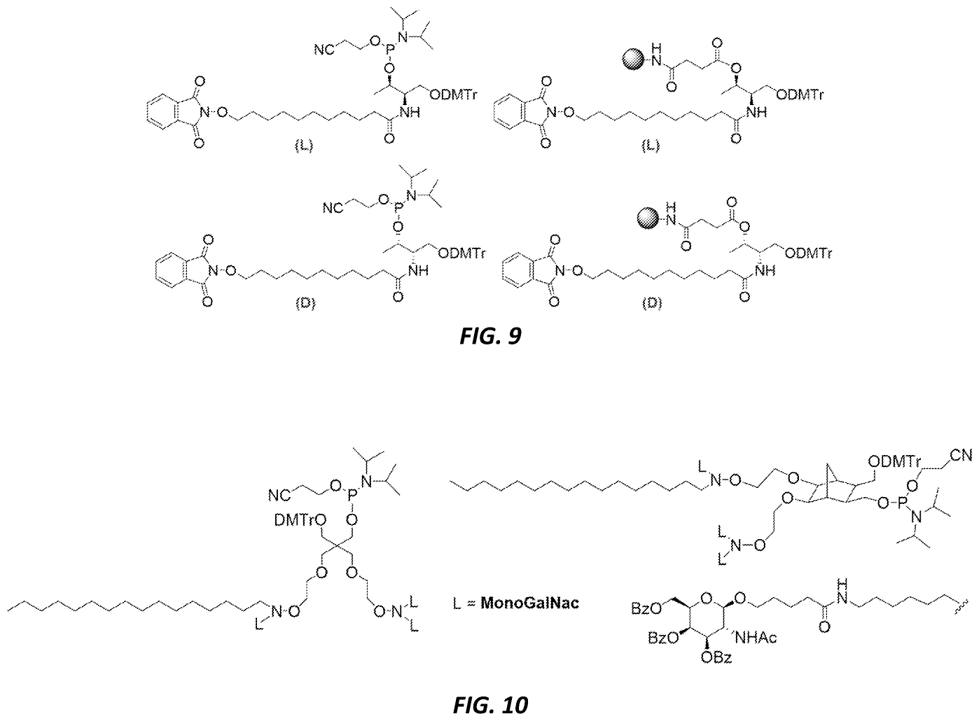

Exemplary non-nucleoside scaffolds for AOCC are shown in FIGS. 7 (Prolinol scaffolds), 8 (Serinol scaffolds), 9 (D- and L-Threoninol scaffolds), and 10 (conjugates derived from Pentaerythritol and Norbornyl scaffolds).

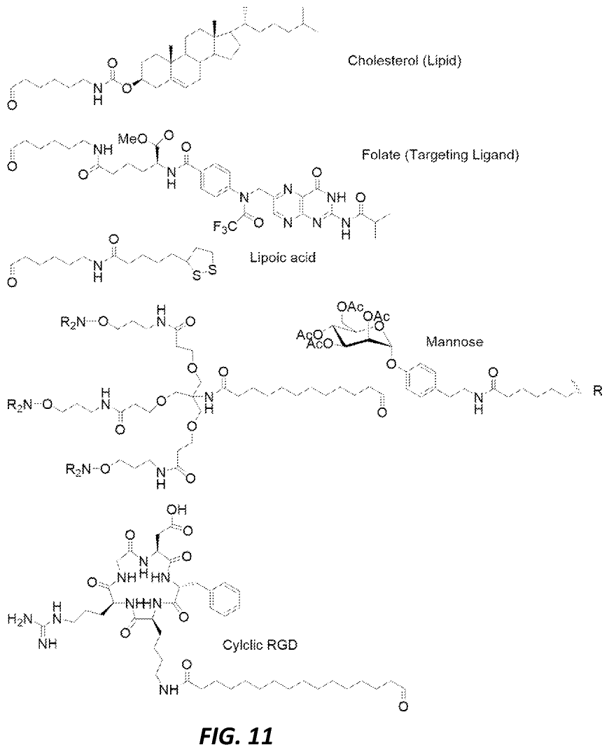

Exemplary ligands with aldehyde linkers and ketone linkers are shown in FIG. 11.

### Oligonucleotide Synthesis

Exemplary target sequences are shown in Table 1.

TABLE 1
Illustrative Target sequences

| Target | Sequences (5′-3′) | Parent Single strands^{§} |
|---|---|---|
| mTTR | A•a•CaGuGuUCUuGcUcUaUaAL96 | A-117799 (S) |
| | u•U•aUaGaGcAagaAcAcUgUu•u•u | A-117800 (AS) |
| mrTTR | u•u•cuugCuCUAuaaaccguguL96 | A-173480 (S) |
| | a•C•acgguuuauagAgCaagaa•c•a | A-173481 (AS) |
| h/cTTR | u•g•ggauUuCAUguaaccaa•g•a | A-555719 (S) |
| | VPu•C•uugGuuAcaugAaAuccca•u•c | A-131902 (AS) |
| SOD1 | c•a•uuuuAaUCCucacucua•a•a | A-637448 (S) |
| | VPu•U•uagAgUGaggaUuAaaaug•a•g | A-444402 (AS) |
| B-cat | U•a•CuGuUgGAUuGaUuCgAaAL10 | A-129065 (S) |
| | VPu•U•uCgAaUcAaucCaAcAgUa•g•c | A-129066 (AS) |
| ApoB | U•g•UgAcAaAUAuGgGcAuCaAL96 | A-127772 (S) |
| | u•U•gAuGcCcAuauUuGuCaCa•a•a | A-127773 (AS) |

^{§}For SOD1 and h/cTTR, original sequences are slightly modified; Potential modification sites in sense strand (Bold and bold italics) and antisense strands (bold italics underlined) are highlighted

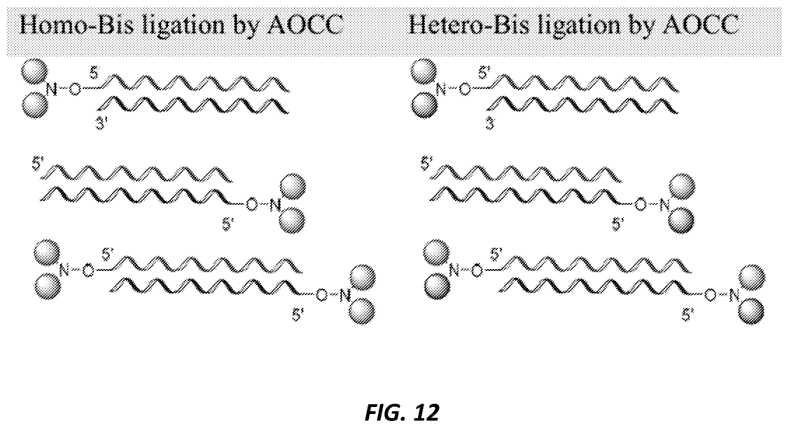

Exemplary modifications sites in oligonucleotides are schematically shown in FIGS. 12-14. FIG. 12 shows single building block incorporation: (i) at 5′-end of sense (red)/antisense (blue) strand and (ii) at 3′-end of sense (red)/antisense (blue) strand. Note: antisense strand AOCC will be in the nucleobases and 2′ 3′ of sugars, not in the 5′ of sugar.

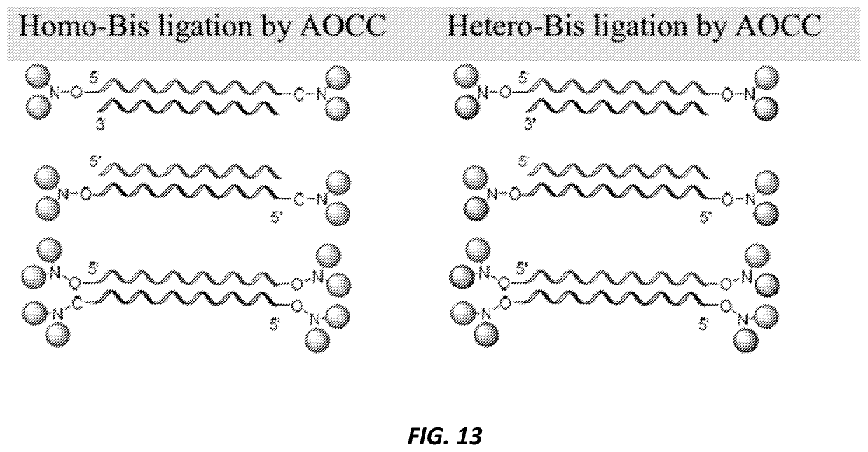

FIG. 13 shows double building block incorporation: (i) 5′-end of sense (red) and 3′-end of antisense (blue) strand; (ii) 3′-end of sense (red) and 5′-end of antisense (blue) strand; (iii) 3′- and 5′-end of sense (red) strand; (iv) 3′- and 5′-end of antisense (blue) strand. Note: antisense strand AOCC will be in the nucleobases and 2′ 3′ of sugars, not in the 5′ of sugar.

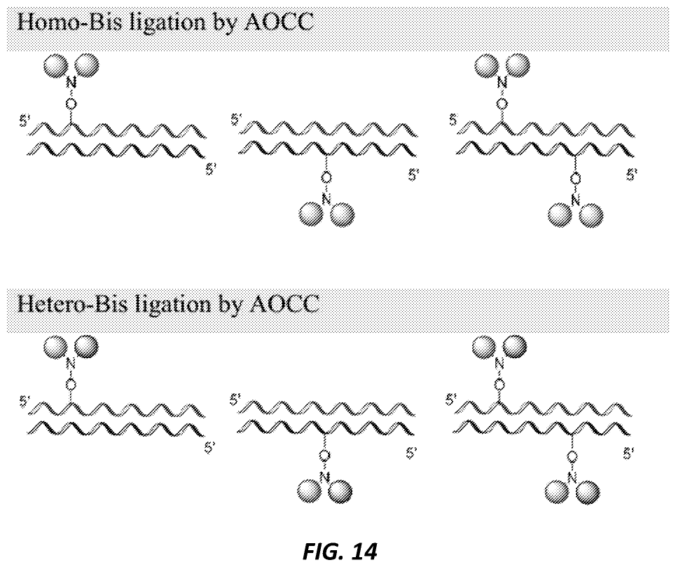

FIG. 14 shows incorporation of single building block at internal position: Single or multiple site modifications are possible either in sense or antisense strands.

### Post-Synthetic Conjugations

Strategy 1: Synthesis of AOCC modified oligonucleotides from O-amino (O—NH₂) precursors (Schemes 7 and 8).

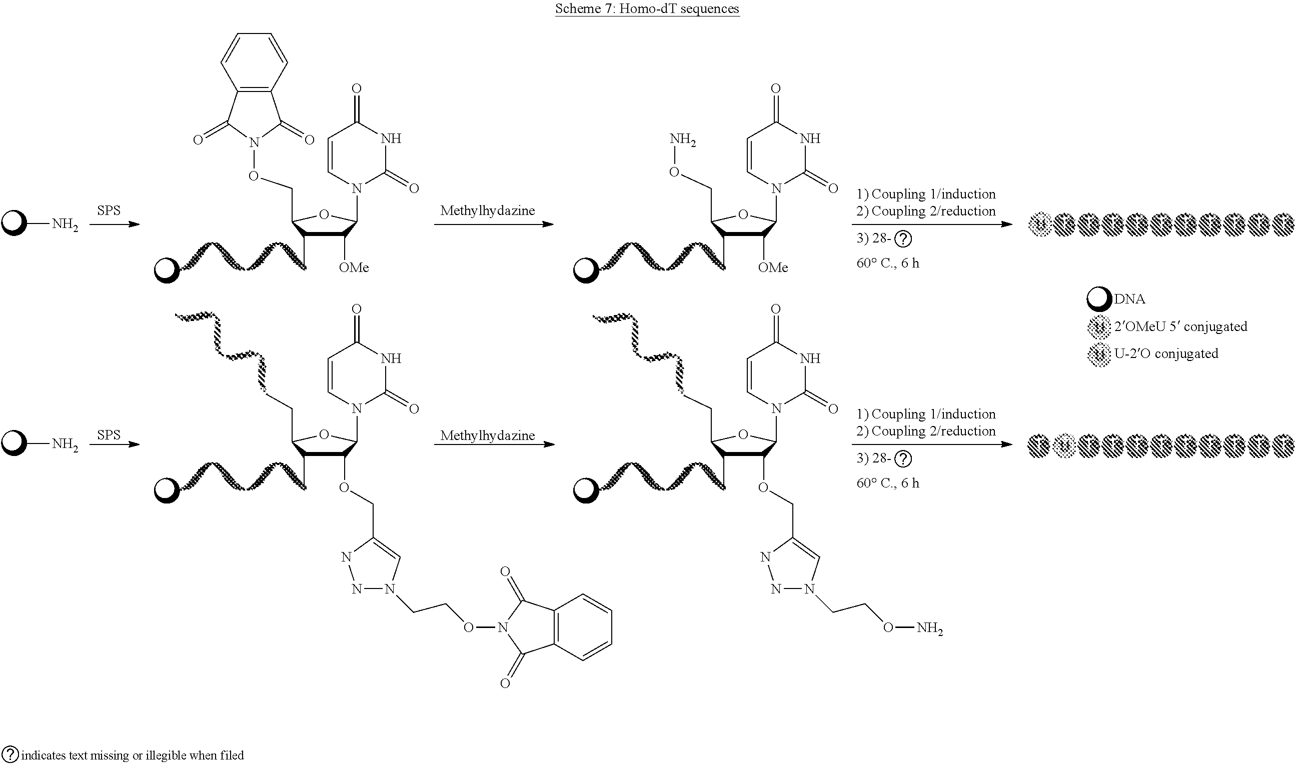

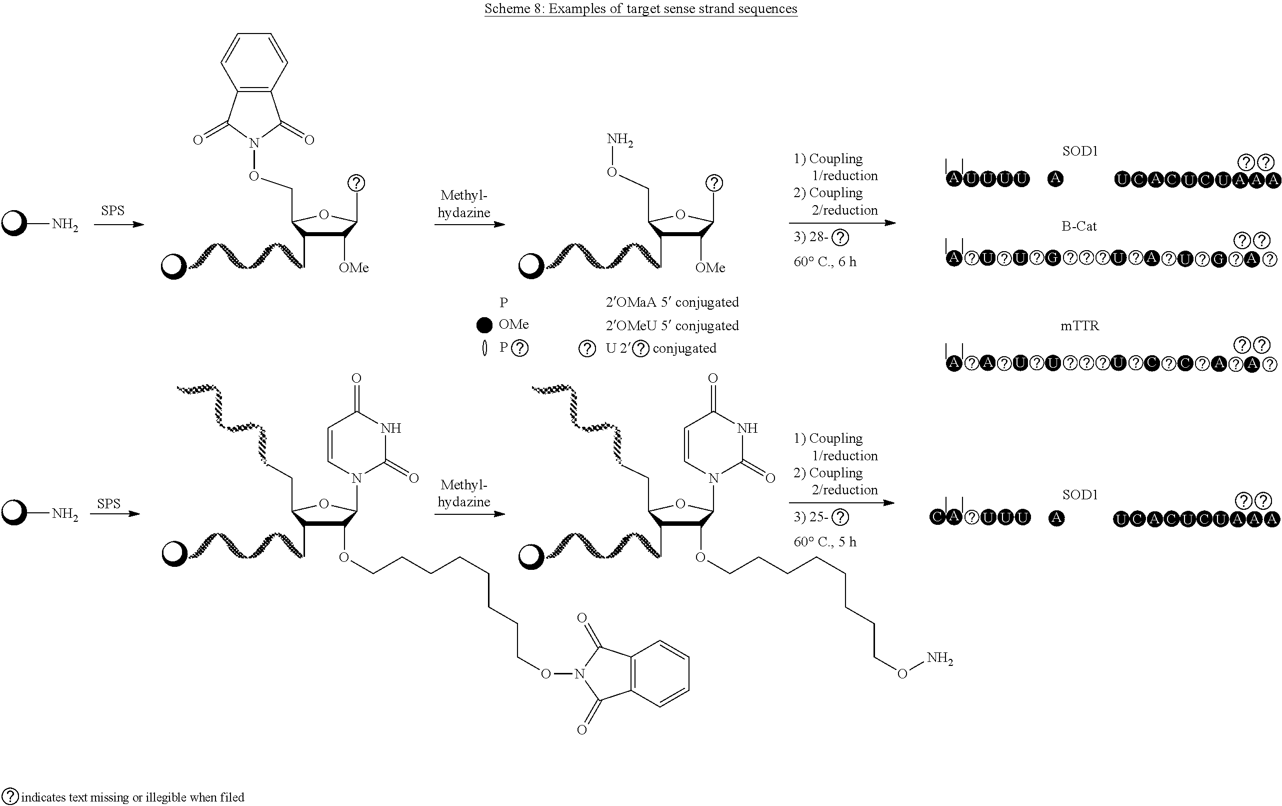

### General Procedure:

A general procedure for synthesis of AOCC modified oligonucleotides from O-amino (O—NH₂) precursors is shown in Scheme 9.

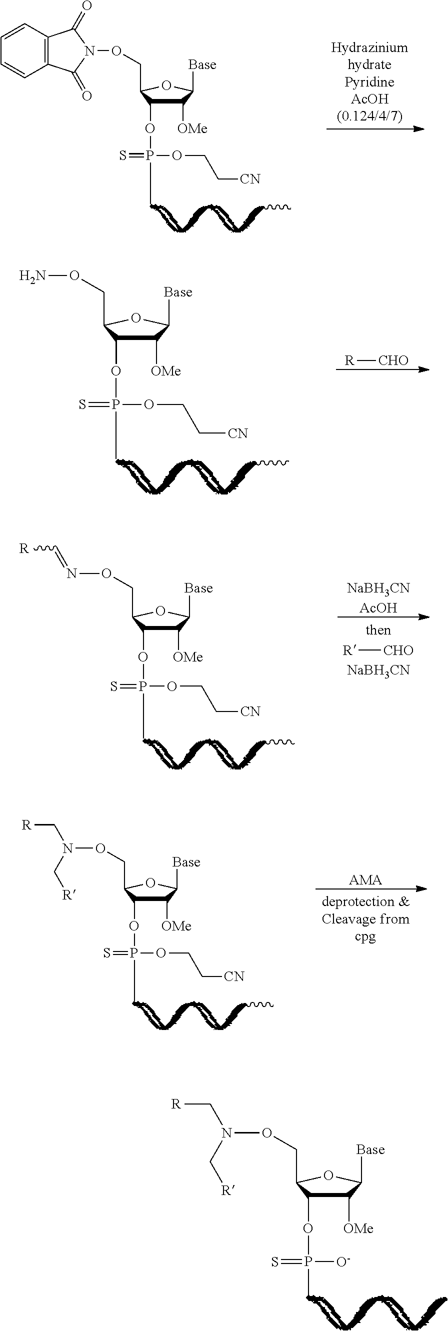

Deprotection of phtalimide: The support-bound oligonucleotides were treated with 0.5 mol/L hydrazine hydrate in Pyridine/AcOH (4:1 v/v) for 1 h at rt. The support was washed with pyridine, MeOH, ACN.

Oxime conjugation: The support-bound oligonucleotides were treated with 1 mol/L aldehyde in DCM for 2 h at rt. The support was washed with DCM, MeOH, ACN.

Reductive amination: The support-bound oligonucleotides were treated with freshly prepared 0.5 M solution of NaCNBH₃ in 75/25 DCM/MeOH with 1% AcOH as an additive for 2 h. The support was washed with DCM, MeOH, ACN.

2^{nd} Reductive amination: The support-bound oligonucleotides were treated with 1 mol/L aldehyde in DCM with 1% AcOH as an additive for 1 h at rt. The reaction vessel was opened and a freshly prepared 1 M solution of NaCNBH₃ in 75/25 DCM/MeOH with 1% AcOH as an additive was added to the resin, to give a final concentration of 0.5 M with respect to both aldehyde and reducing agent. The reaction was again capped and allowed to shake for 2 h. The support was washed with DCM, MeOH, ACN.

Deprotection & Cleavage from CPG: ONs were cleaved from solid support and deprotected using ammonium hydroxide at 30° C. for 17 h. After filtration through a nylon syringe filter (0.45 μm), ONs were either stored for purification. ONs were purified using anion-exchange high-performance liquid chromatography (IEX-HPLC) using an appropriate gradient of mobile phase (0.15 M NaCl, 10% MeCN and 1.0 M NaBr, 10% MeCN) and desalted using size-exclusion chromatography with water as an eluent. ONs were then quantified by measuring the absorbance at 260 nm using the following extinction coefficients: (A=13.86, T/U=7.92, C=6.57, and G=10.53). The purity and identity of modified ONs were verified by analytical anion exchange chromatography mass spectrometry.

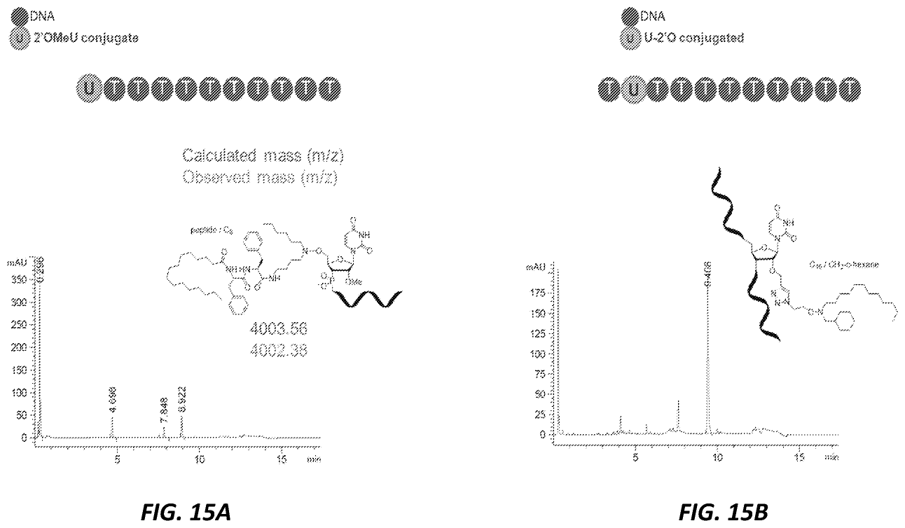

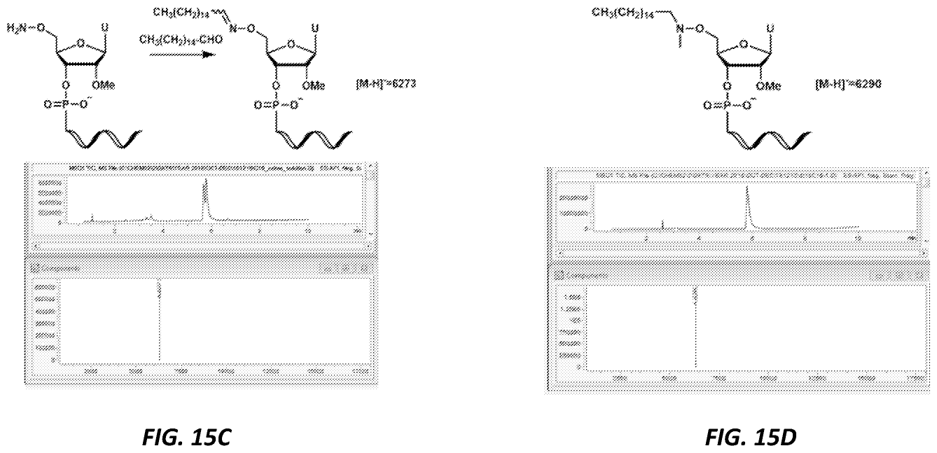

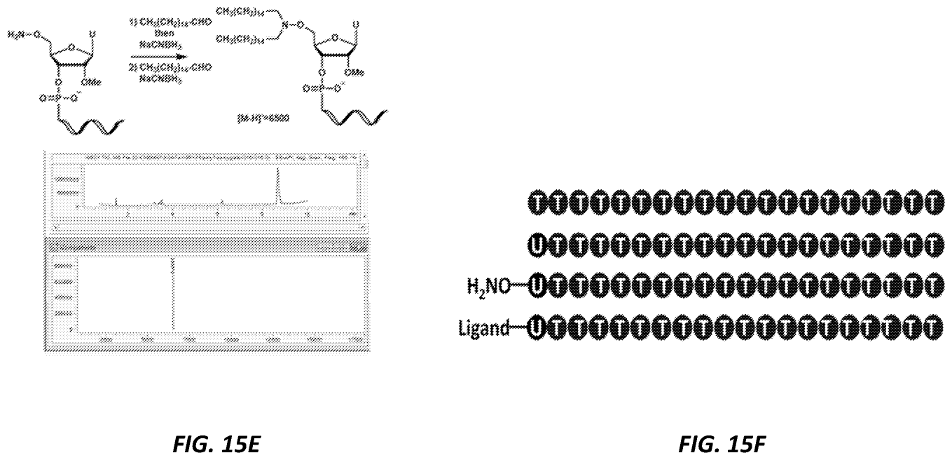

Results for Homo-dT10 and dT20 sequences are shown in FIGS. 15A-15F and summarized in Table 2. Sequences and mass spectroscopy characterization of target sequence conjugates are summarized in Table 3.

TABLE 2
Sequences and mass spectroscopy characterization of dT sequence conjugates

| | | | Mass (m/z) | |
|---|---|---|---|---|
| Position | Strand | Sequence 5′-3′ | Calc. | Obs. |
| 5′ | A− | u(peptide/C6)dTdTdTdTdTdTdTdTdTdT | 4003.564 | 4002.38 |
| 5′ | A− | u(acetonilideC16/Me)dTdTdTdTdTdTdTdTdTdT | 3687.349 | 3685.86 |
| internal | A− | dTu(C12/chexane)dTdTdTdTdTdTdTdTdTdT | 3995.55 | 3994.06 |

deoxy-Thymidine (dT), 2′ C12 and cyclohexane conjugate (u(C12/chexane)), 5′C12 and cyclohexane 2′OMe uridine conjugate (u(peptide/C6)), 5′acetonilideC16 and Me 2′OMe uridine conjugate (u(acetonilideC16/Me)).

TABLE 3
Sequences and mass spectroscopy characterization of target sequence conjugates

| | | | Mass (m/z) | |
|---|---|---|---|---|
| Target | Strand | Sequence 5′-3′ | Calc. | Obs. |
| mTTR | A− | a(retinal ox)•a•CaGuGuUCUuGcUcUaUaA(L) | 8916.315 | |
| mTTR | A- | a(C16/biotin)•a•CaGuGuUCUuGcUcUaUaA(L) | 9464.666 | |
| mTTR | A− | a(dilinoleyl/Me)•a•CaGuGuUCUuGcUcUaUaA(L) | 9174.634 | |
| SOD1 | A− | c•a•u(C16/biotin)uuuAfaUfCfCfucacucua•a•a | 7721.039 | |
| Bcat | A− | u(triGalNAc/C16)•a•uuuuAfaUfCfCfucacucua•a•a | 8905.466 | |
| ApoB | A− | u(triGalNAc/C16)•a•uuuuAfaUfCfCfucacucua•a•a | 8902.561 | |

Uppercase, lowercase, and uppercase red bold letters represent LNA, 2′-OMe sugar modifications and 2,6-diaminopurine nucleoside, respectively, to Adenosine (A), Cytidine (C), Guanosine (G), Uridine (U), 5′ trans-retinal oxime-2′OMe adenosine (a(retinal ox)), 5′C16-biotin 2′OMe adenosine (a(C16/biotin)), 5′dilinoleyl-Me 2′OMe adenosine (a(dilinoleyl/Me)), 2′C16-biotin uridine (u(C16/biotin)), 5′triGalNAc-C16 2′OMe uridine (u(triGalNAc/C16)). Phosphorothioate linkages are indicated by the “•” symbol and TriGalNAc ligand by (L).

Some exemplary oligonucleotide sequences for on-support and in-solution AOCC are shown in Table 4.

TABLE 4
Exemplary sequences for on-support and in-solution AOCC

| | Strand | |
|---|---|---|
| Target | (sense) | Sequence 5′-3′ |
| β-cat | A-3346443 | Q341sasCfuGfuUfgGfAfUfuGfaUfuCfgAfsasAf |
| β-cat | A-3748013 | Y276sasCfuGfuUfgGfAfUfuGfaUfuCfgAfsasAf |
| β-cat | | Y280sasCfuGfuUfgGfAfUfuGfaUfuCfgAfsasAf |
| β-cat | A-3346444 | Y277sasCfuGfuUfgGfAfUfuGfaUfuCfgAfsasAf |
| mTTR | A-3346442 | Y301sasCfaGfuGfuUfCfUfuGfcUfcUfaUfaAfL96 |
| mTTR | | Y@sasCfaGfuGfuUfCfUfuGfcUfcUfaUfaAfL96 |
| SOD-1 | | csasuuuY240AfaUfCfCfucacucuasasa |
| SOD-1 | | csasuuuY293AfaUfCfCfucacucuasasa |
| mTTR | | AfsasCfaGfuGfuUfCfUfuGfcUfcUfaUfaAfL413 |
| mTTR | | AfsasCfaGfuGfuUfCfUfuGfcUfcUfaUfaAfL@ |
| β-cat | | Y275sasCfuGfuUfgGfAfUfuGfaUfuCfgAfsasAf |

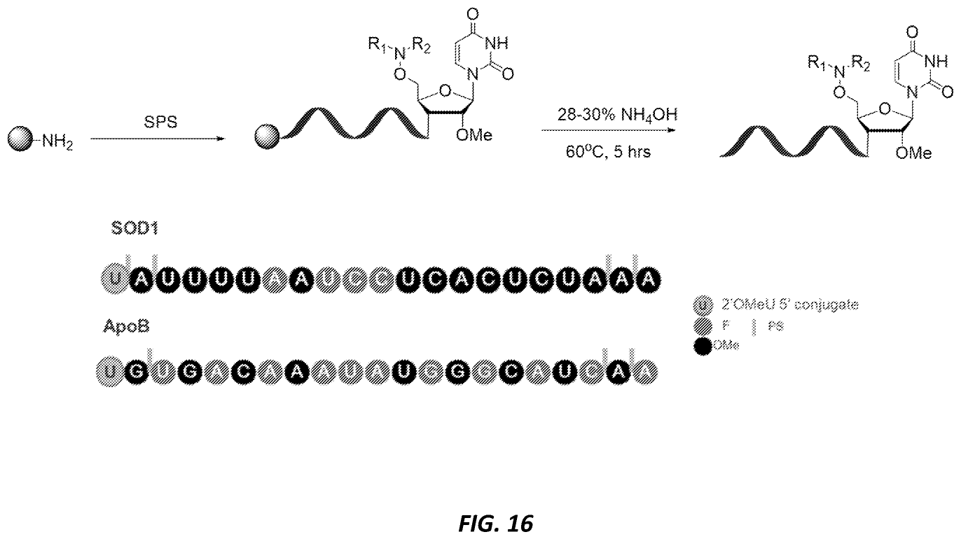

Strategy 2: Synthesis of AOCC modified oligonucleotides from AOCC-conjugate building blocks (FIG. 16).

Oligosynthesis: Oligonucleotides used for the exonuclease assay were synthesized on an ABI-394 and those used for in vitro efficacy assays were synthesized on a MerMade 192 synthesizer on 1-mol scale using universal or custom supports. A solution of 0.25 M 5-(S-ethylthio)-1H-tetrazole in acetonitrile (CH₃CN) was used as the activator. The solutions of commercially available phosphoramidites and synthesized 5′-(R)—C-methyl-guanosine phosphoramidities were 0.15 M in anhydrous CH₃CN or ACN/DMF (9:1, v/v). The 5′-(S)—C-methyl-guanosine phosphoramidities were 0.15 M in anhydrous 15% DCM in CH₃CN. The oxidizing reagent was 0.02 M I₂ in THF/pyridine/H₂O. The detritylation reagent was 3% dichloroacetic acid in CH₂Cl₂. After completion of the automated synthesis, the oligonucleotide was manually released from support and deprotected using a mixture of aqueous MeNH₃ (40% wt) at 60° C. for 12 min or using 28-30% ammonium hydroxide solution at 60° C. for 5 h. After filtration through a 0.45-μm nylon filter, oligonucleotides were either purified or, for oligonucleotides containing ribose sugars, the 2′ hydroxyl was deprotected by treatment with Et₃N·3HF at 60° C. for 30 min. Oligonucleotides were purified using IEX-HPLC using an appropriate gradient of mobile phase (buffer A: 0.15 M NaCl, 10% CH₃CN; buffer B 1.0 M NaBr, 10% MeCN) and desalted using size-exclusion chromatography with water as an eluent. Oligonucleotides were then quantified by measuring the absorbance at 260 nm. Extinction coefficients were calculated using the following extinction coefficients for each residue: A, 13.86; T/U, 7.92; C, 6.57; and G, 10.53 M-‘cm’. The purity and identity of modified ONs were verified by analytical anion exchange chromatography and mass spectrometry, respectively.

After the trityl-off synthesis using the MerMade 192, columns were incubated with 150 L of 40% aqueous methylamine for 30 min at room temperature, and solutions were drained via vacuum into a 96-well plate. After repeating the incubation and draining with a fresh portion of aqueous methylamine, the plate containing the crude oligonucleotides was sealed and shaken at room temperature for 60 min to completely remove all protecting groups. In the case of RNA, the 2′ hydroxyl was deprotected by treating with Et₃N·3HF at 60° C. for 60 min. Precipitation of the crude oligonucleotides was accomplished via the addition of 1.2 mL of ACN/EtOH (9:1, v/v) to each well, followed by centrifugation at 3000 rpm for 45 min at 4° C. The supernatant was removed from each well, and the pellets were resuspended in 950 μL of 20 mM aqueous NaOAc. Oligonucleotides were desalted over a GE Hi-Trap desalting column (Sephadex G25 Superfine) using water as an eluant. The identities and purities of all oligonucleotides were confirmed using ESI-MS and IEX-HPLC, respectively.

For oligonucleotides synthesized using the ABI 394, the manufacturer's standard protocols were used for cleavage and deprotection. Crude oligonucleotides were purified using strong anion exchange with phosphate buffers (pH 8.5) containing NaBr. The identities and purities of all oligonucleotides were confirmed using ESI-LC/MS and IEX-HPLC respectively.

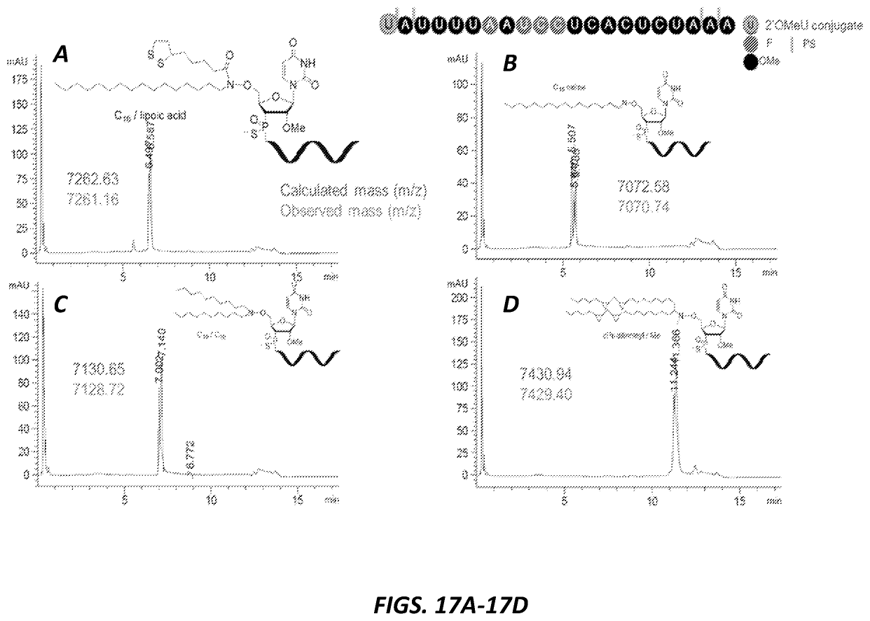

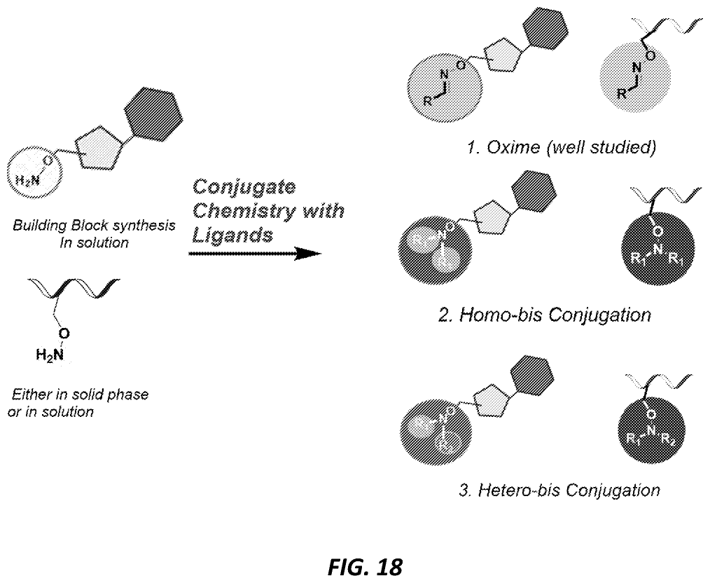

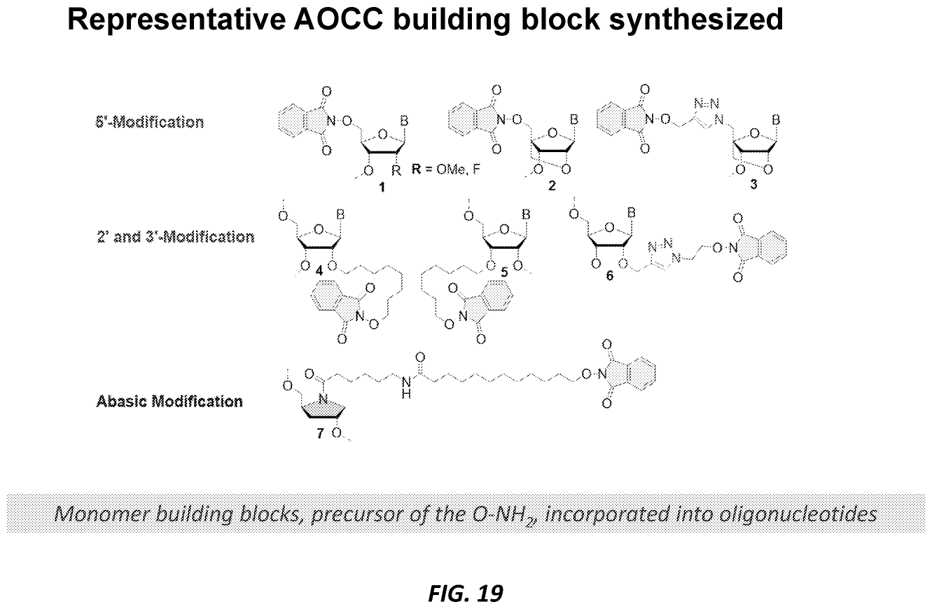

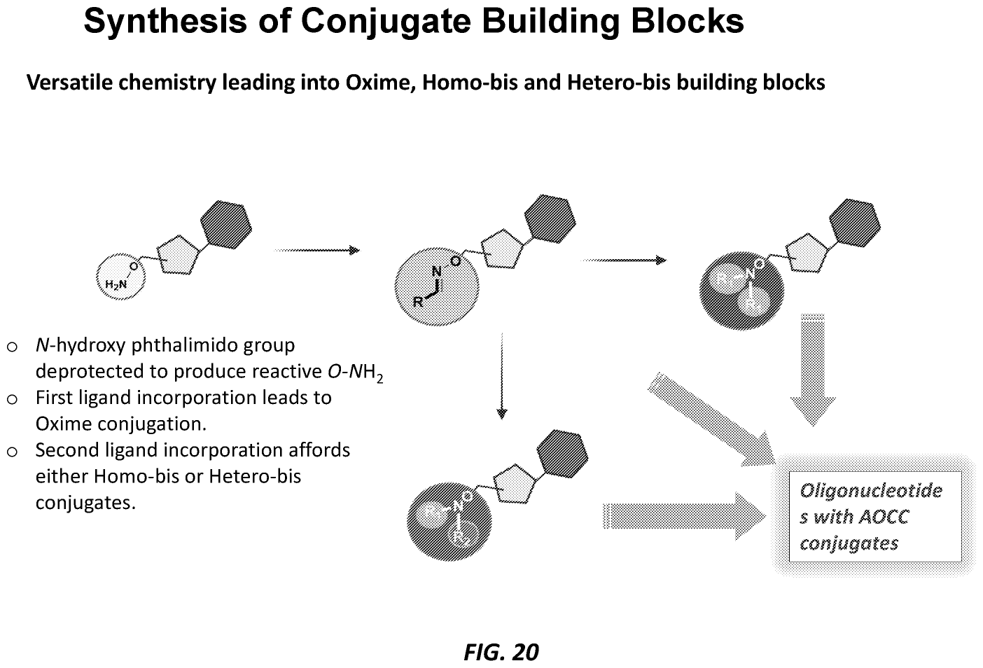

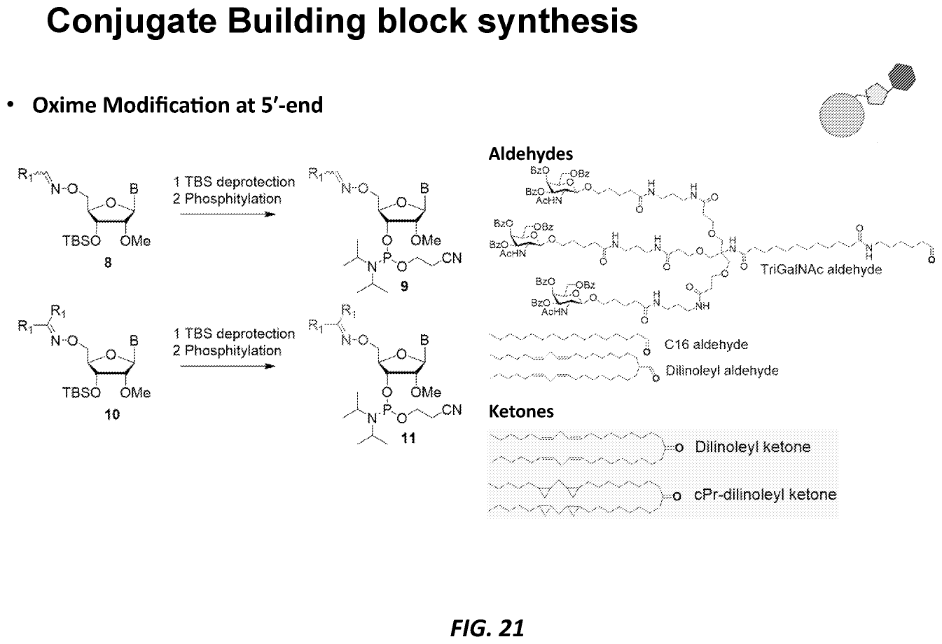

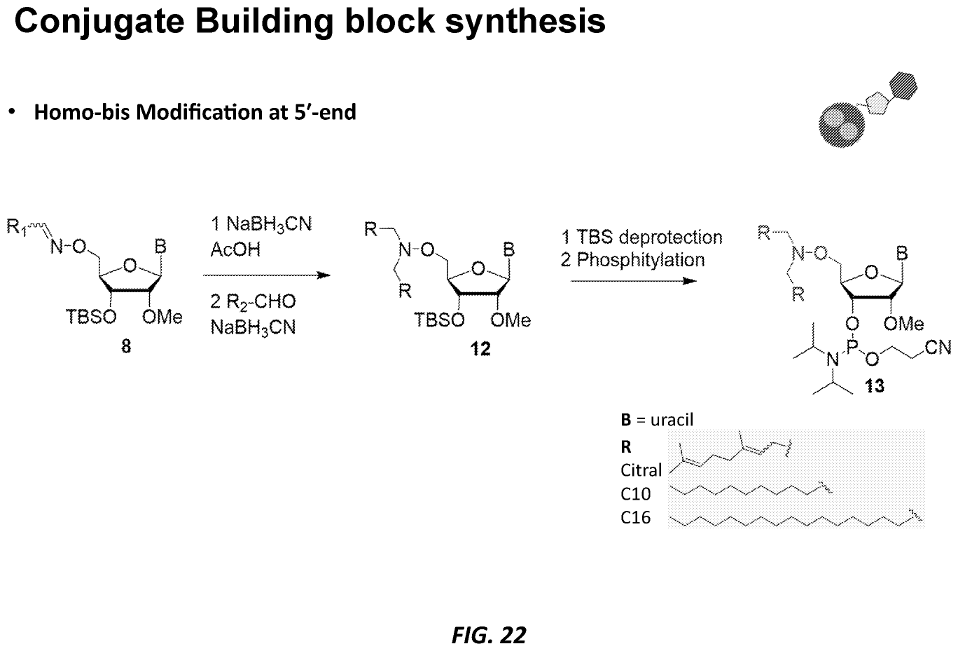

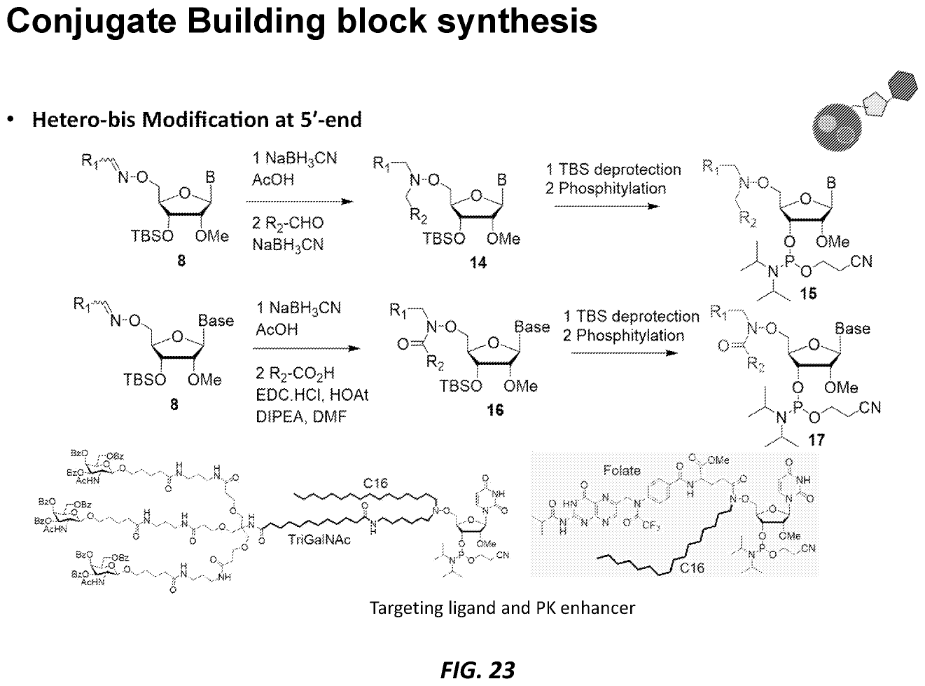

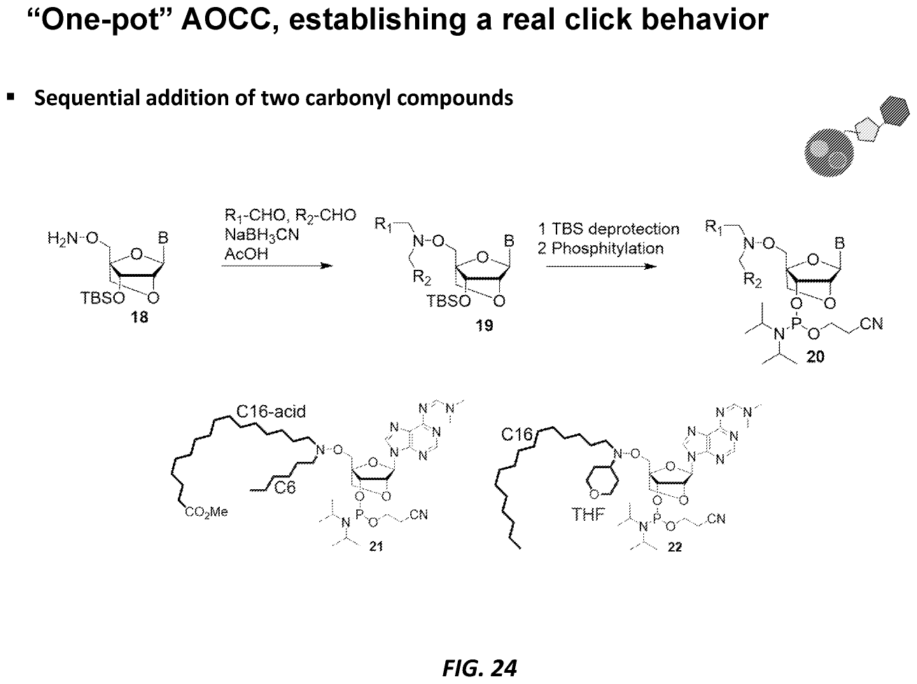

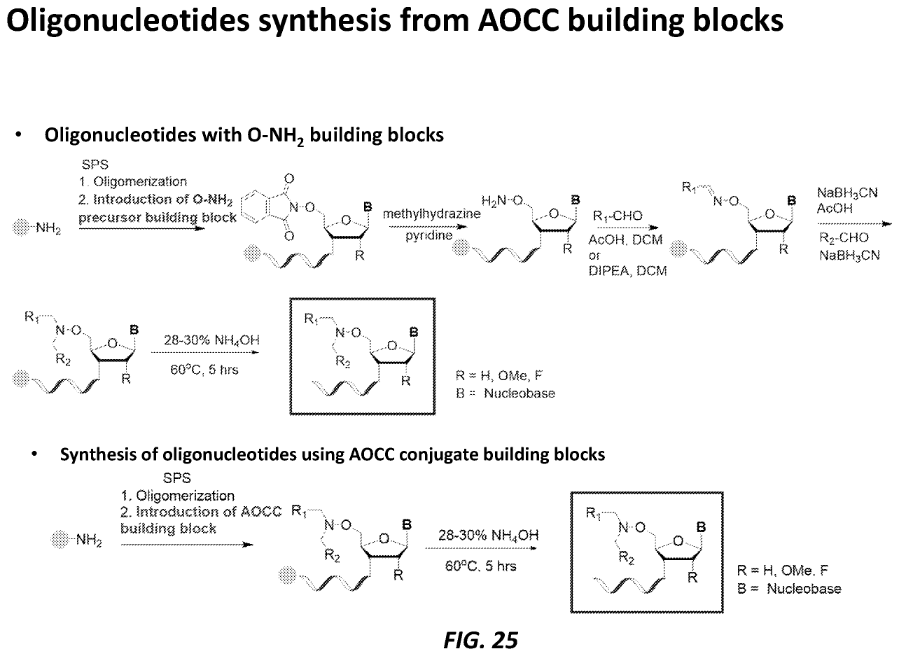

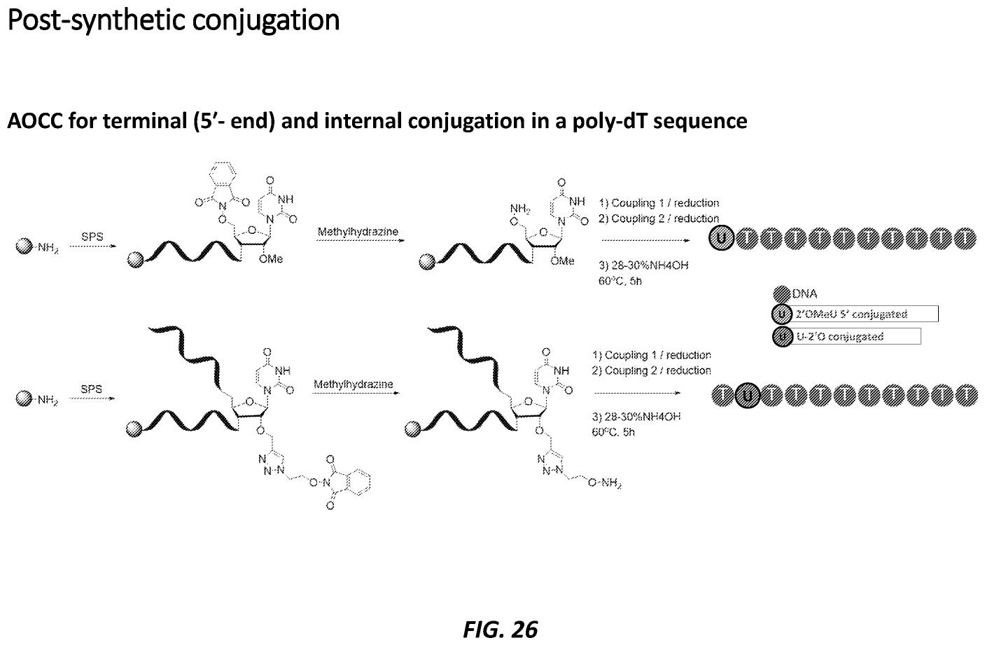

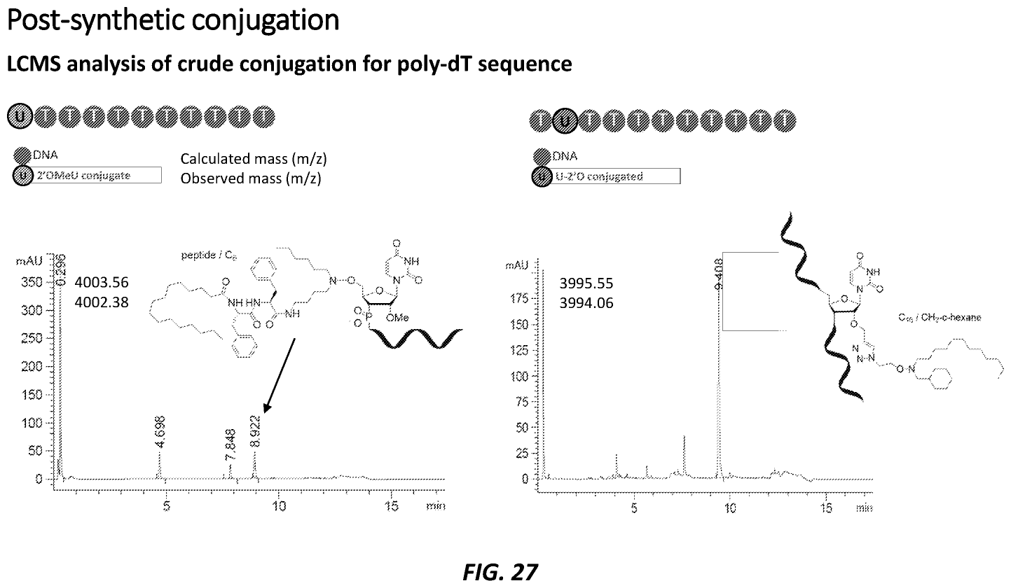

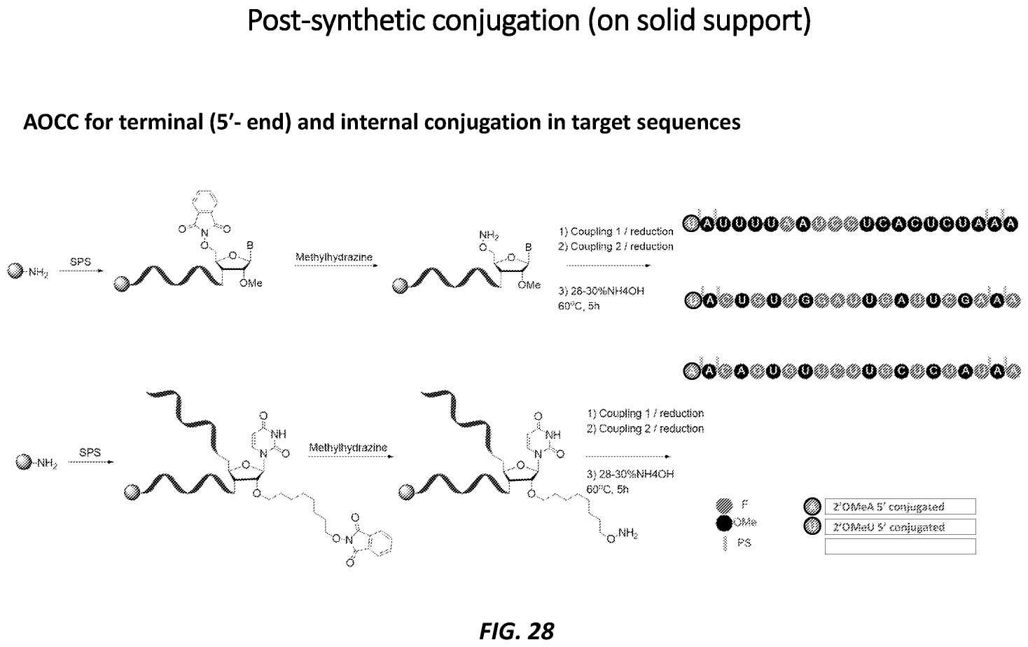

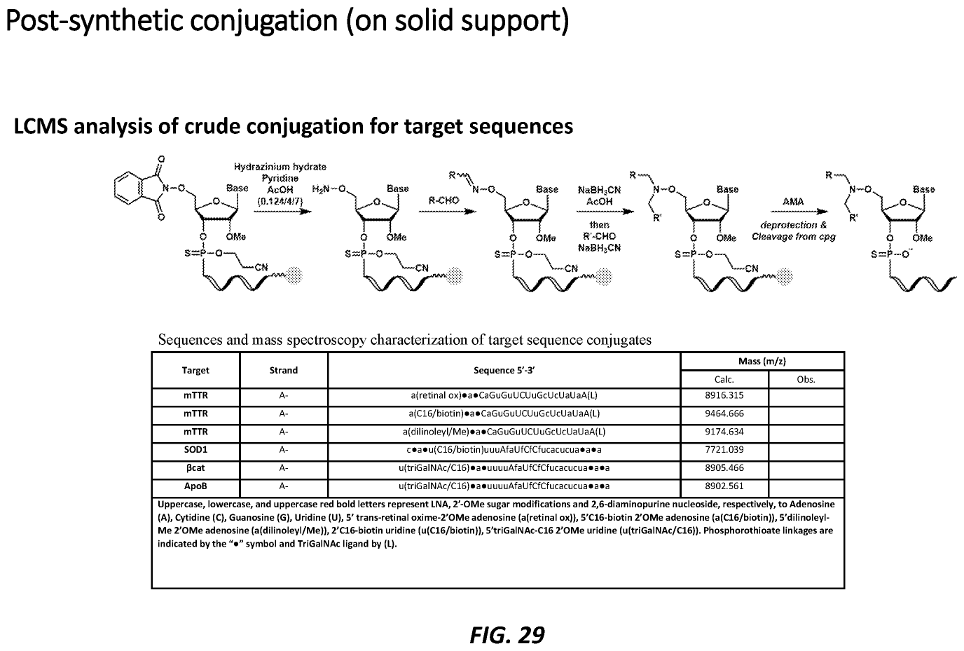

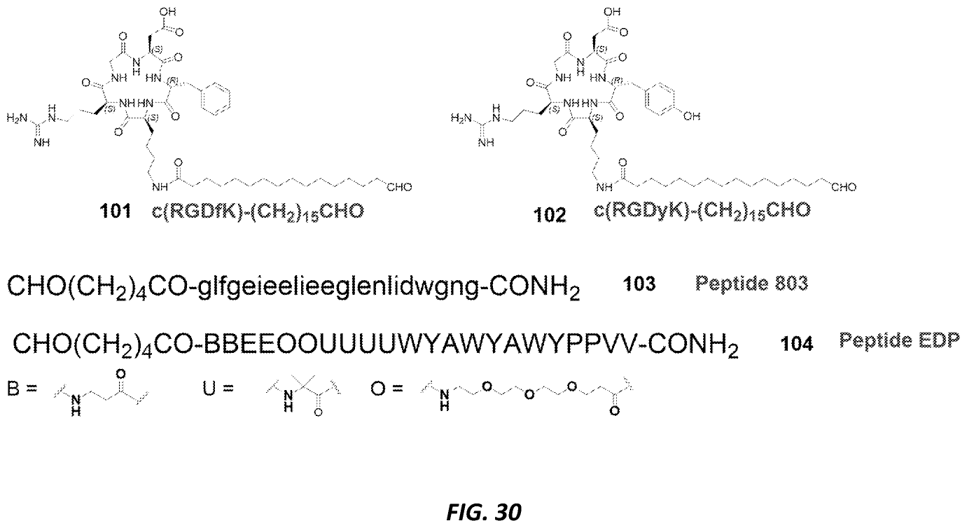

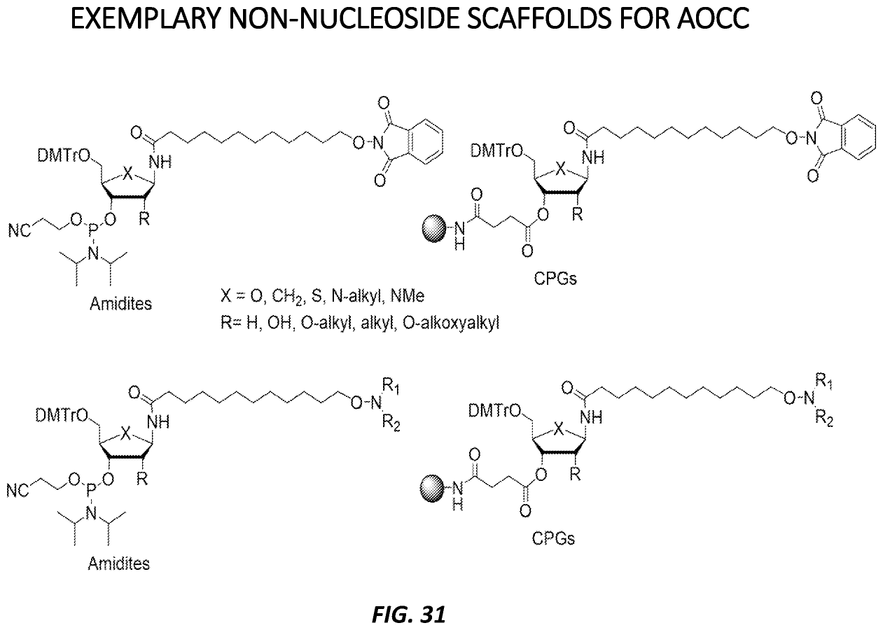

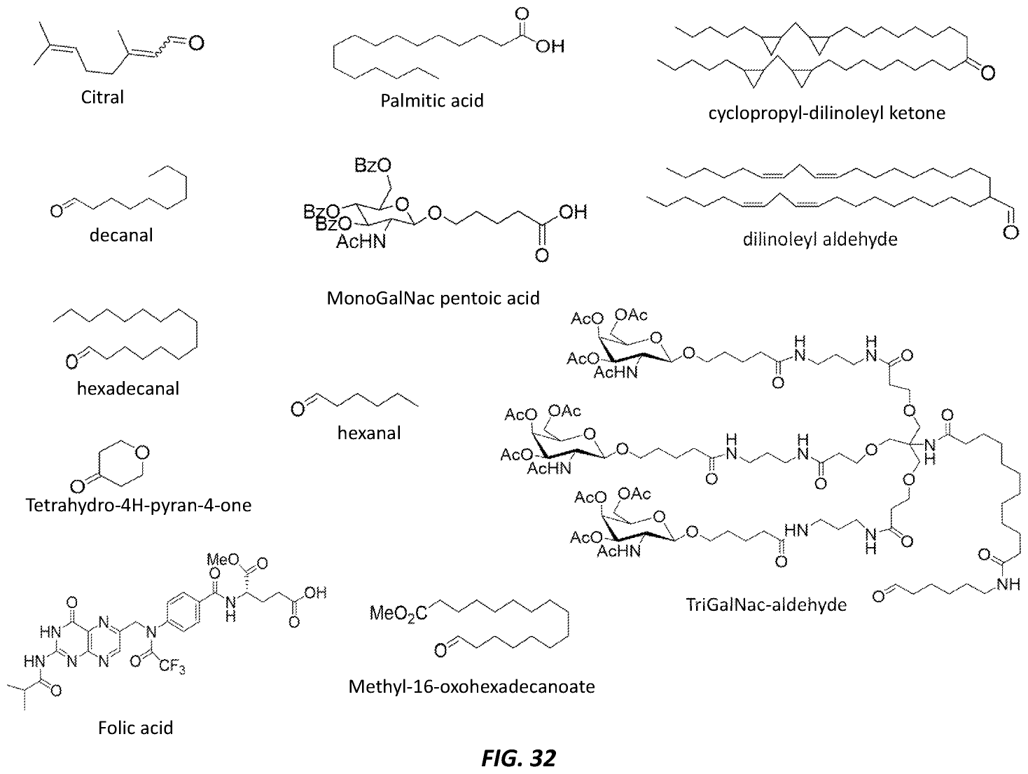

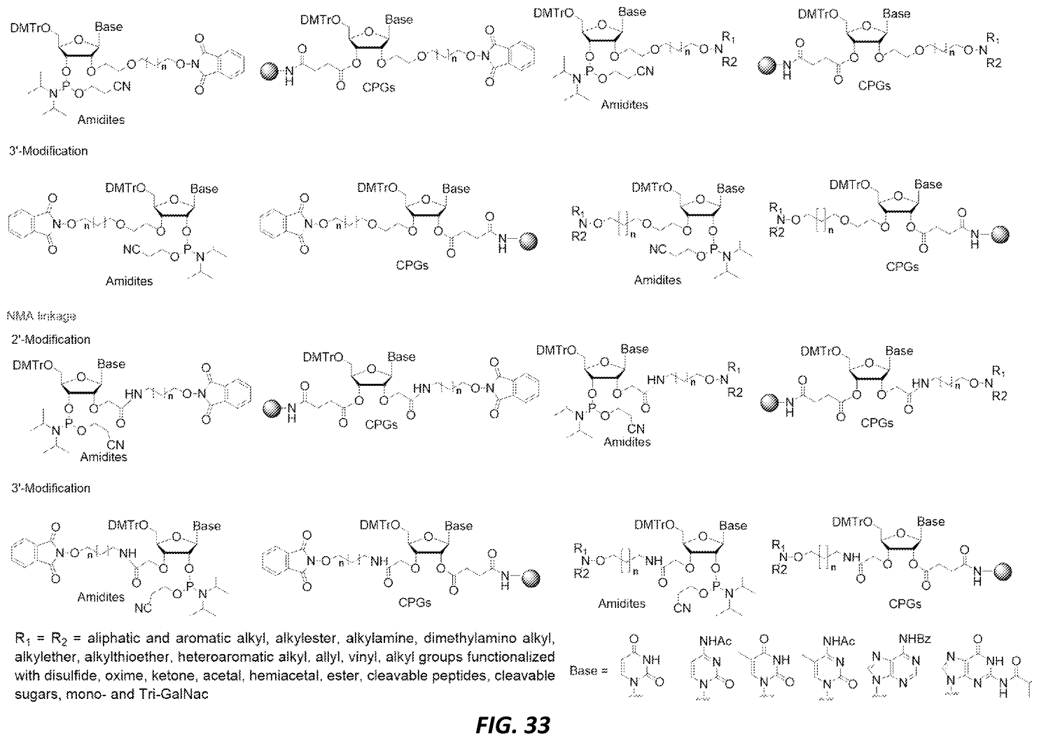

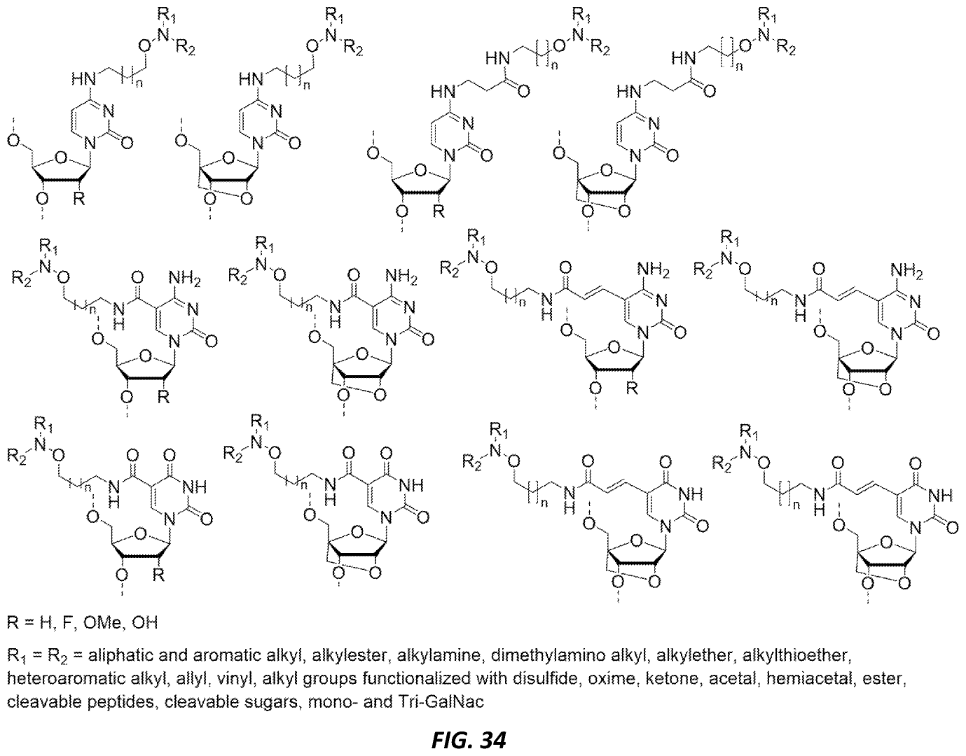

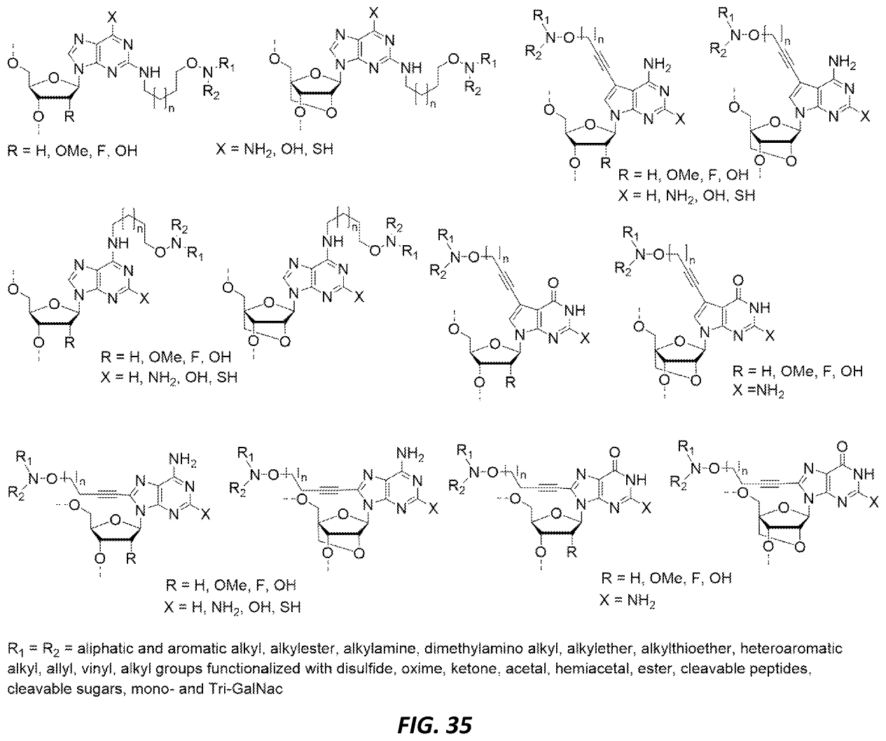

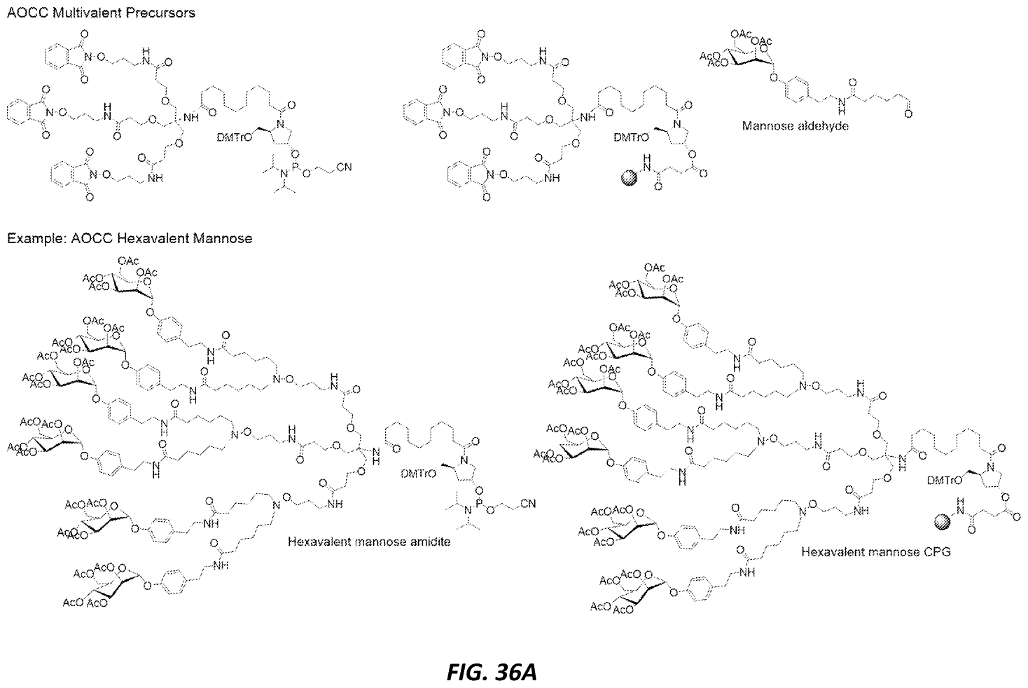

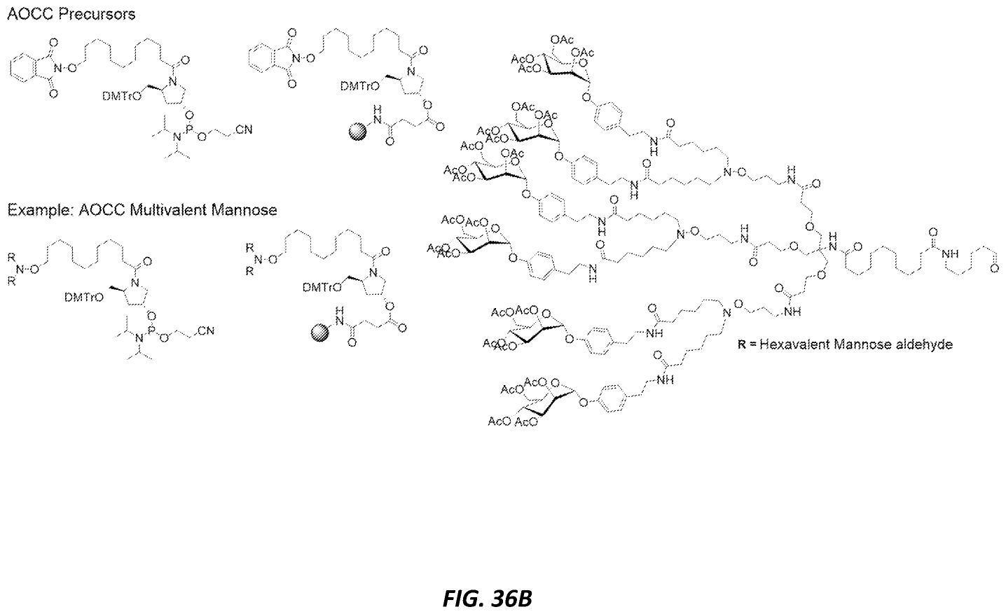

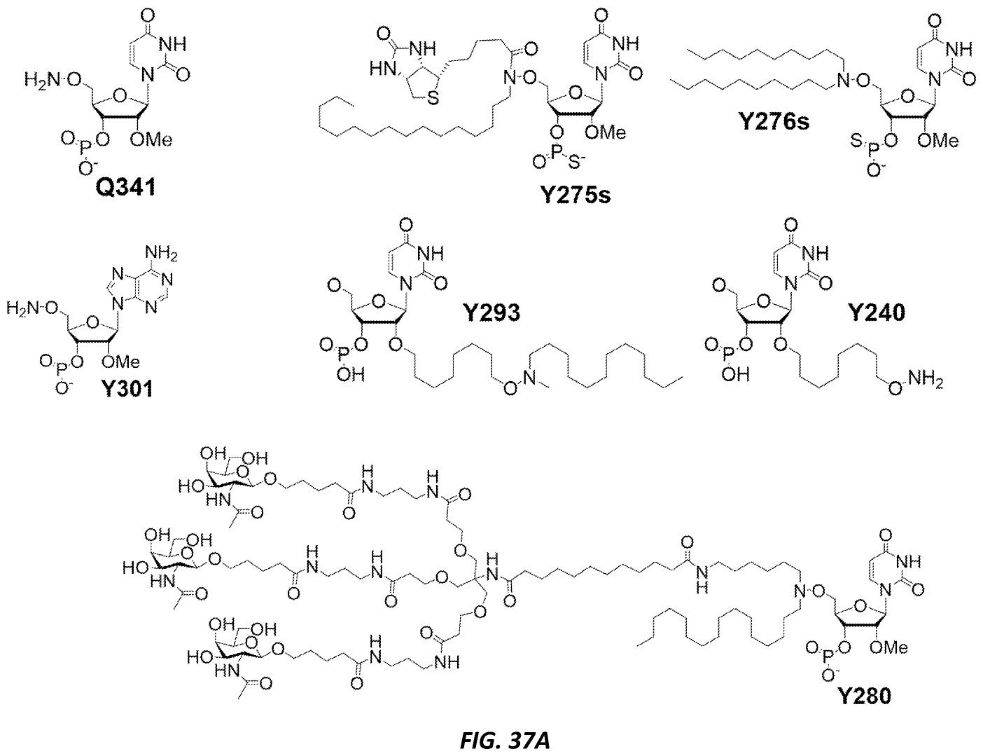

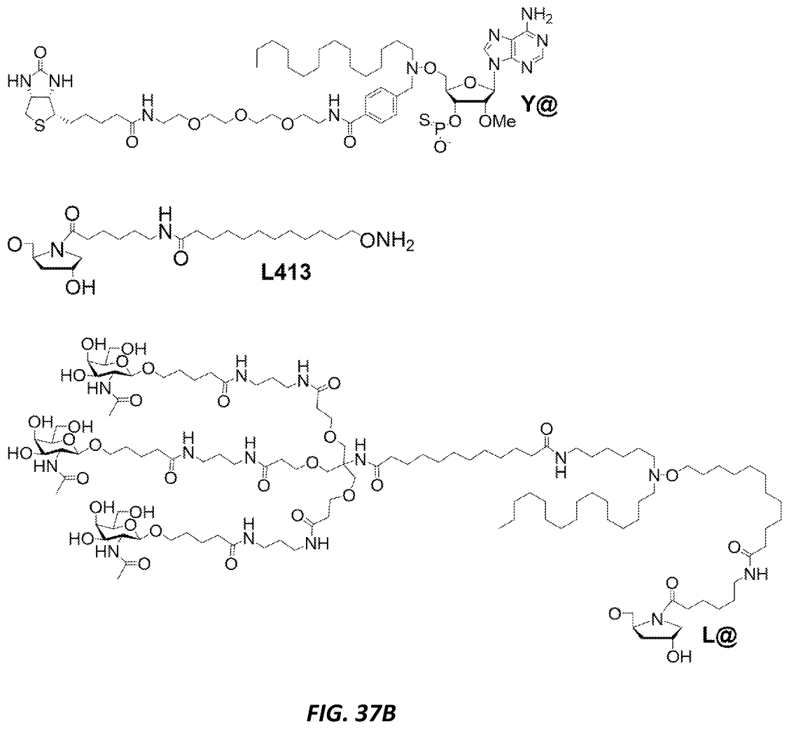

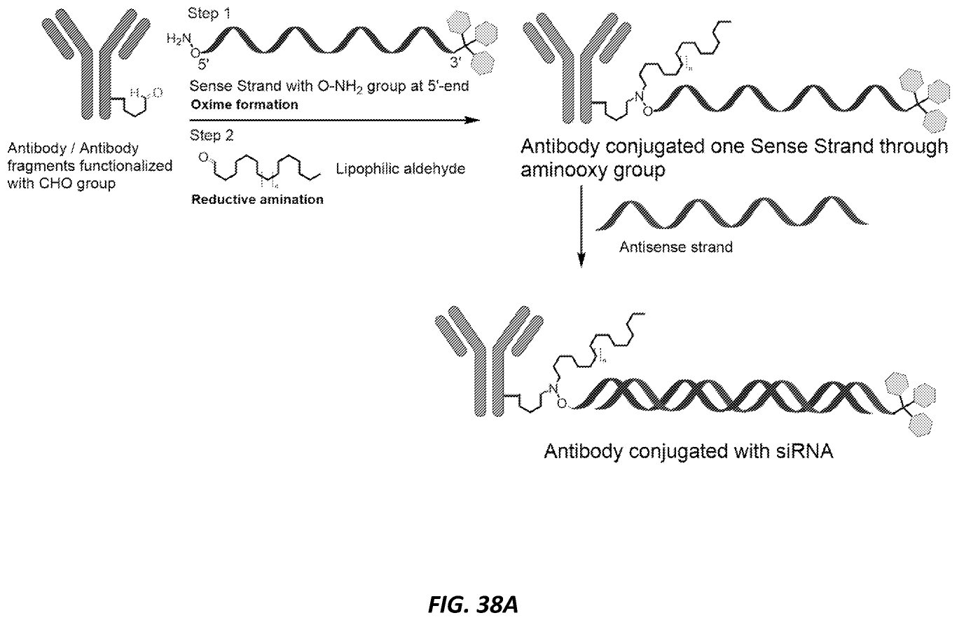

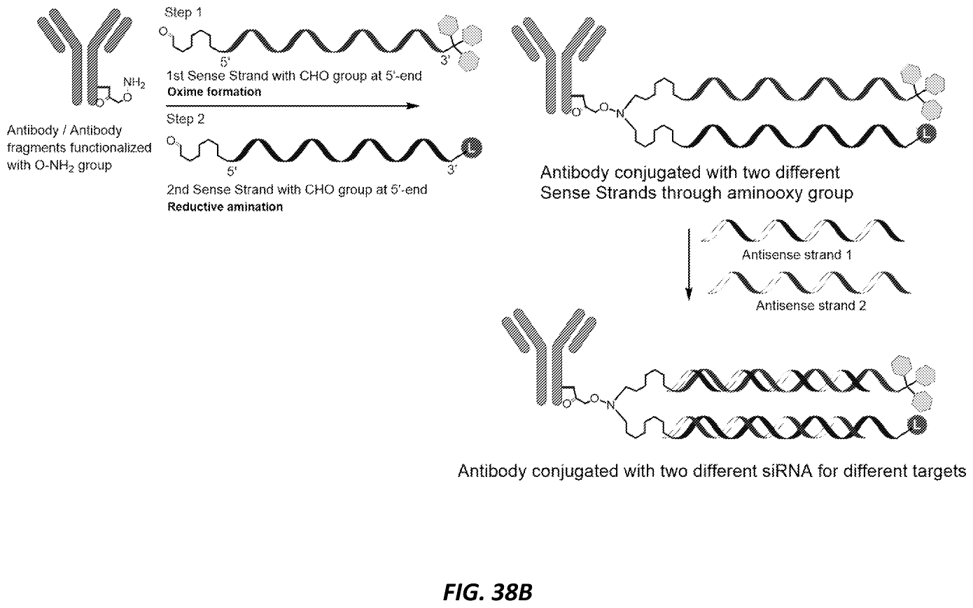

Results are shown in FIGS. 17A-17D and summarized in Table 5.

TABLE 5
Sequences and mass spectroscopy characterization of target sequence conjugates using
AOCC-conjugated building blocks

| | Sense | | Mass (M − H)⁻ | | |
|---|---|---|---|---|---|
| Target | Strand | Sequence 5′-3′ | Chemistry | Calc. | Obs. |
| SOD1 | A-3341126 | Y278•a•uuuuAaUCCucacucua•a•a | oxime | 7072.99 | 7071.22 |
| SOD1 | A-3341125 | Y276•a•uuuuAaUCCucacucua•a•a | bis-homo | 7131.12 | 7129.65 |
| SOD1 | A-3341127 | Y281•a•uuuuAaUCCucacucua•a•a | bis-hetero | 7431.65 | 7429.90 |
| SOD1 | A-3341128 | Y279•a•uuuuAaUCCucacucua•a•a | bis-hetero | 7263.31 | 7261.77 |
| βcat | A- | Y277•a•CuGuUgGAUuGaUuCgA•a•A | bis-hetero | 7589.3 | 7588.07 |
| Apo-B | A- | Y280•g•UgAcAaAUAuGgGcAuC•a•A | bis-hetero | 8920.0 | 8916.50 |

Uppercase italicized and lowercase letters represent 2′-F and 2′-OMe nucleosides, respectively.
Phosphorothioate linkages are indicated by the “•” symbol.
AOCC-modified building blocks in the context of 2′-OMe-uridine are Y276 (5′-bis-C10), Y277 (5′-folate-C16), Y278 (5′-C16 oxime), Y279 (5′-lipoic acid-C16), Y280 (5′-triGalNAc-C16), and Y281 (5′-cyclopropyldilinoleyl-Me).

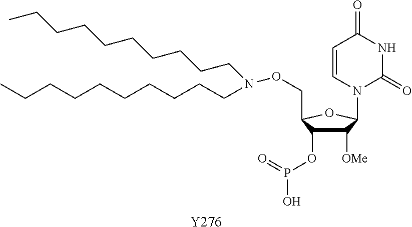

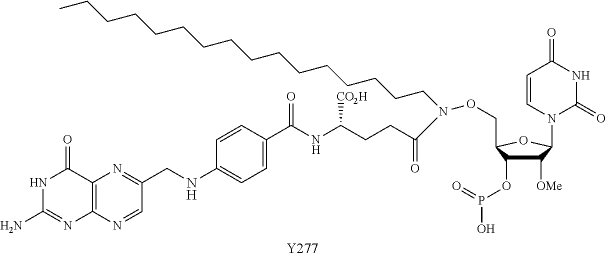

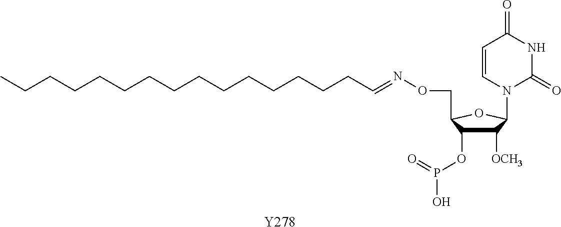

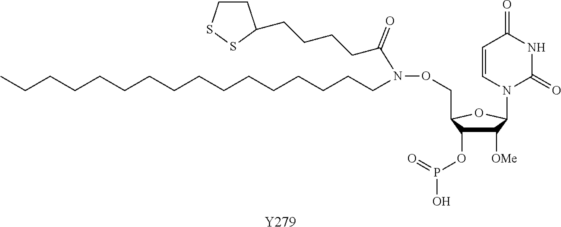

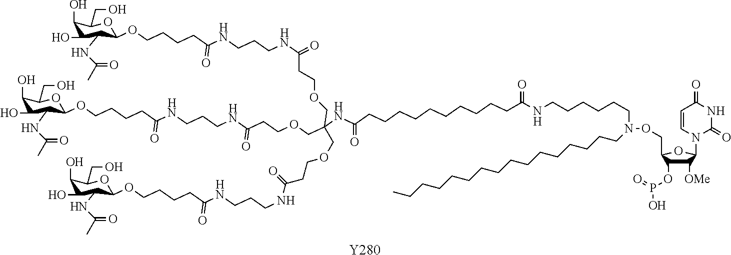

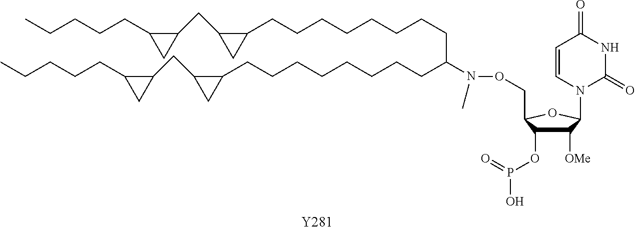

Strategy 3: Synthesis of AOCC modified oligonucleotide with hydrolyzable linkers in solution (Scheme 10).

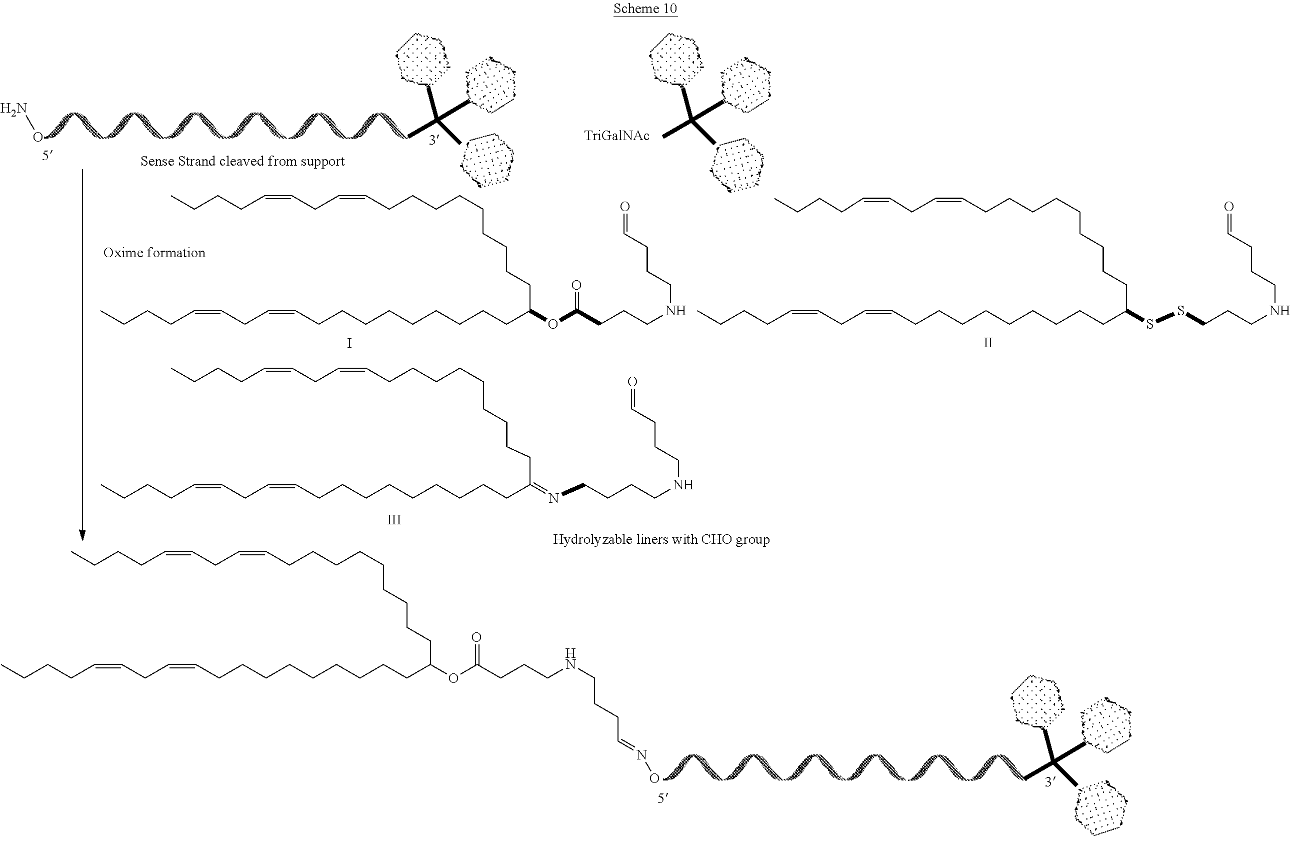

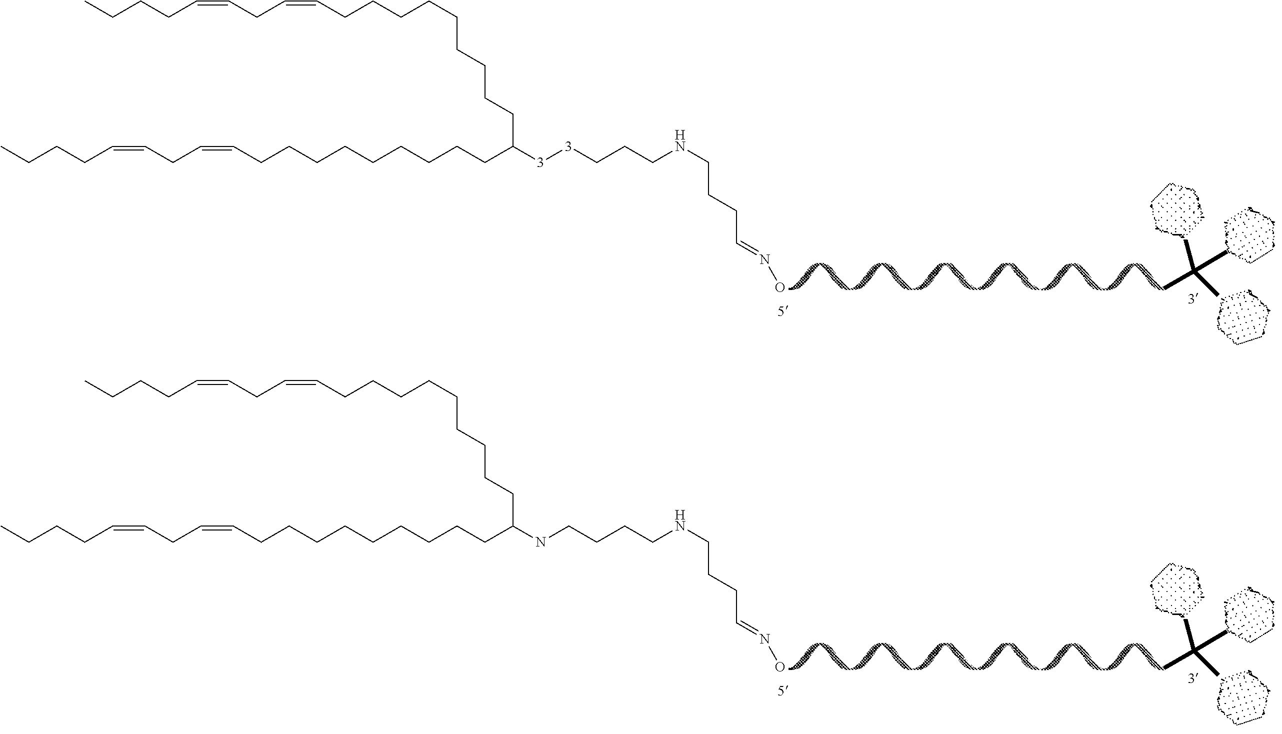

Reaction and scheme: Oligonucleotide was synthesized on an ABI-394 on 1-mol scale using universal or custom supports. A solution of 0.25 M 5-(S-ethylthio)-1H-tetrazole in acetonitrile (CH₃CN) was used as the activator. The solutions of commercially available phosphoramidites and synthesized 5′-(R)—C-methyl-guanosine phosphoramidities were 0.15 M in anhydrous CH₃CN or ACN/DMF (9:1, v/v). The 5′-(S)—C-methyl-guanosine phosphoramidities were 0.15 M in anhydrous 15% DCM in CH₃CN. The oxidizing reagent was 0.02 M I₂ in THF/pyridine/H₂O. The detritylation reagent was 3% dichloroacetic acid in CH₂C₂. After completion of the automated synthesis, the support-bound oligonucleotides were treated with 0.5 mol/L hydrazine hydrate in Pyridine/AcOH (4:1 v/v) for 1 h at rt to deprotect phtalimido group. The support was washed with pyridine, MeOH, ACN. The oligonucleotide was manually released from support and deprotected using ammonium hydroxide at 30° C. for 5 h. After filtration through a nylon syringe filter (0.45 μm). ON was purified using anion-exchange high-performance liquid chromatography (IEX-HPLC) using an appropriate gradient of mobile phase (0.15 M NaCl, 10% MeCN and 1.0 M NaBr, 10% MeCN) and desalted using size-exclusion chromatography with water as an eluent. ON was then quantified by measuring the absorbance at 260 nm using the following extinction coefficients: (A=13.86, T/U=7.92, C=6.57, and G=10.53). The purity and identity of modified ONs were verified by analytical anion exchange chromatography mass spectrometry. Oxime conjugation with (5Z,8Z,29Z,32Z)-heptatriaconta-5,8,29,32-tetraen-19-yl-4-((4,4-diethoxybutyl)amino)butan -oate (IV) was accomplished after treating the purified oligomer with 1 mol/L aldehyde in 20% DMF in water containing 2% acetic acid. The reaction mixture was kept at rt for 5 hrs and then dialyzed by 500 Dalton dialysis cut-off bag through overnight. Aqueous solution was filtered through a nylon syringe filter (0.45 μm), ON was purified using anion-exchange high-performance liquid chromatography (IEX-HPLC) using an appropriate gradient of mobile phase (0.15 M NaCl, 10% MeCN and 1.0 M NaBr, 10% MeCN) and desalted using size-exclusion chromatography with water as an eluent. Collected fractions were lyophilized to afford the oligonucleotide.

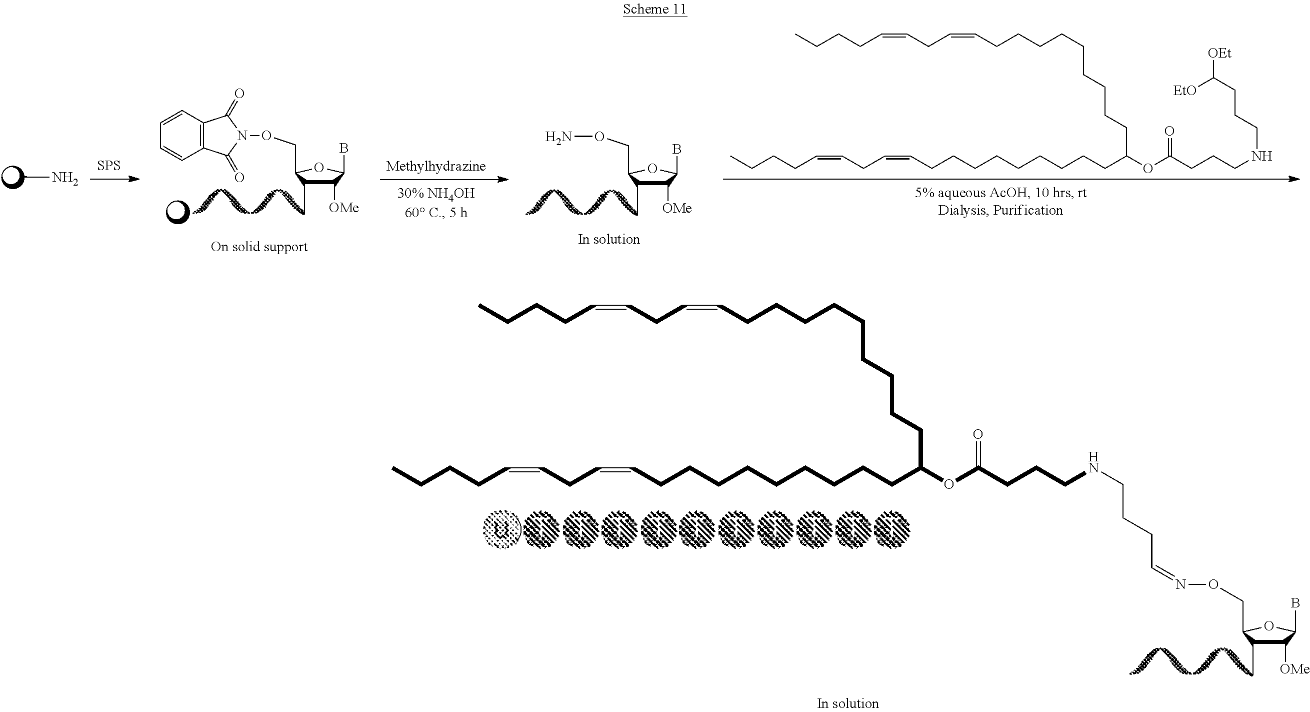

### Variation of Strategy 3

A: Synthesis of AOCC modified bi-siRNA constitution in solution (Schemes 12-14)

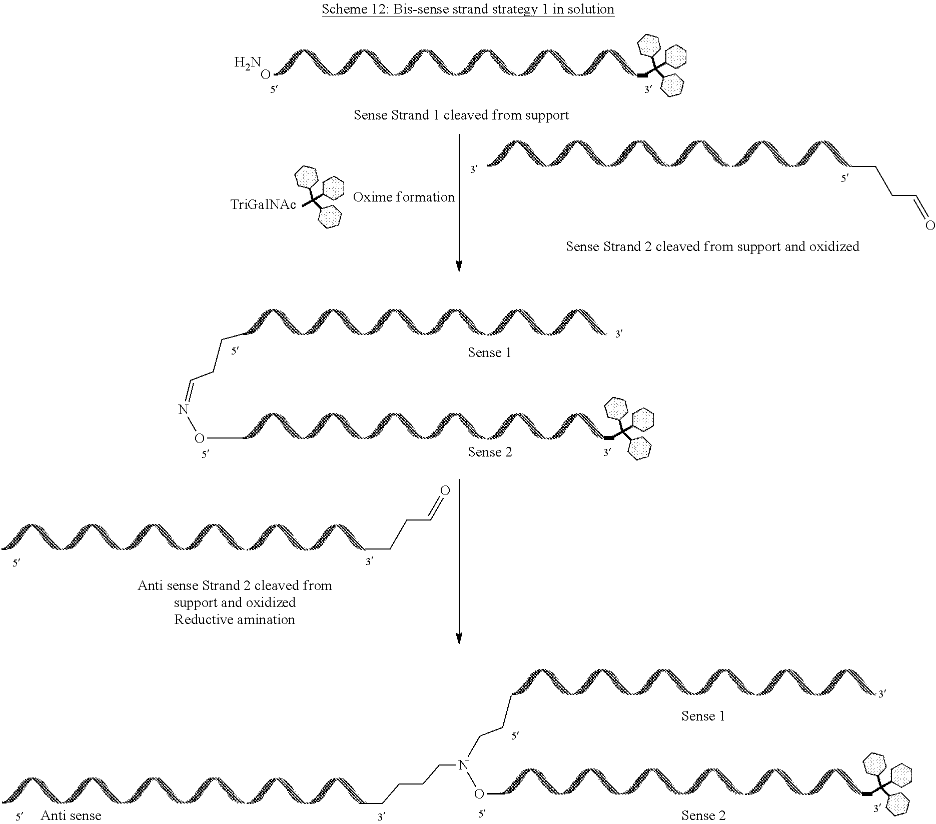

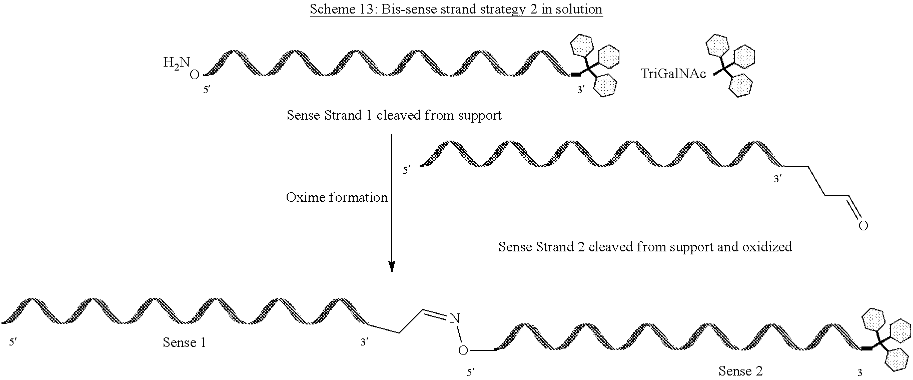

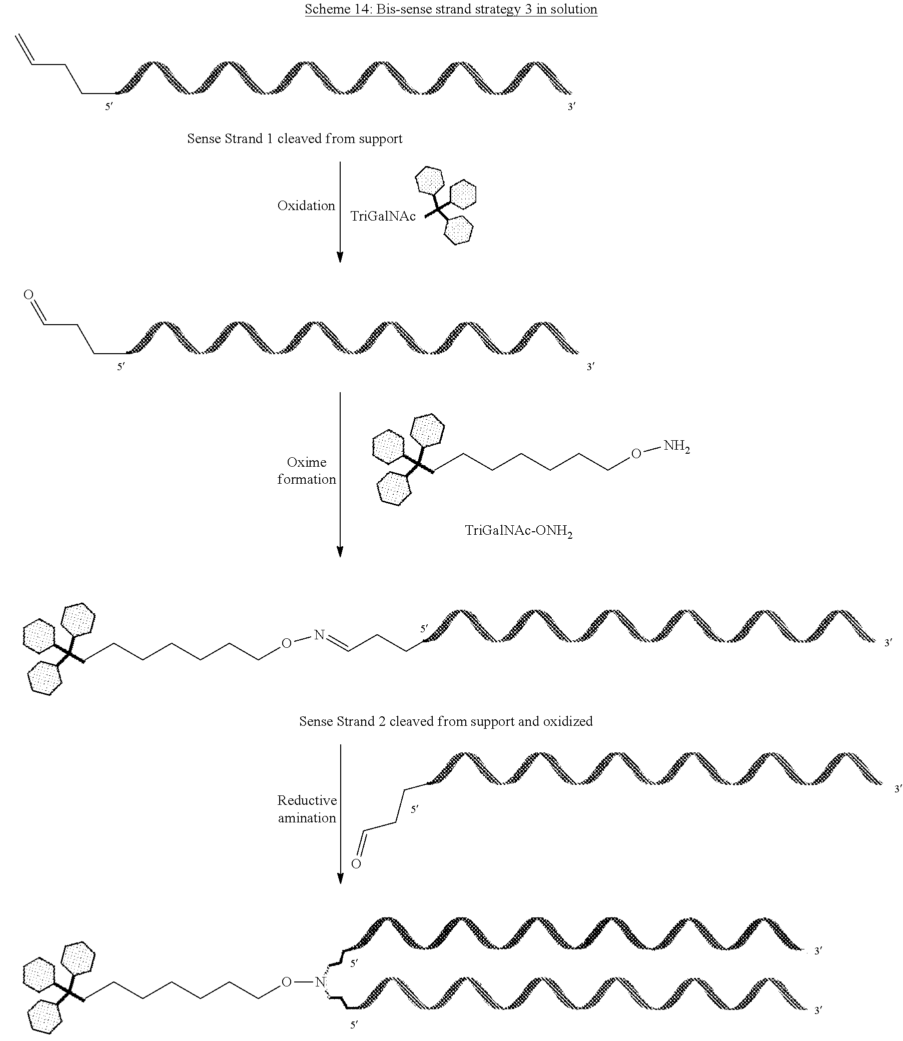

### Variation of Strategy 3

B: Scheme 15, reaction scheme between antibodies and siRNAs. Schematic representation of the pepsin digestion, the reduction of F(ab′)₂, and the conjugation. Preparation of antibody Fab′ siRNA conjugate²⁷. The formylglycine (fGly) residue produced by formylglycine generating enzyme (FGE) in CxPxR sequences²⁸.

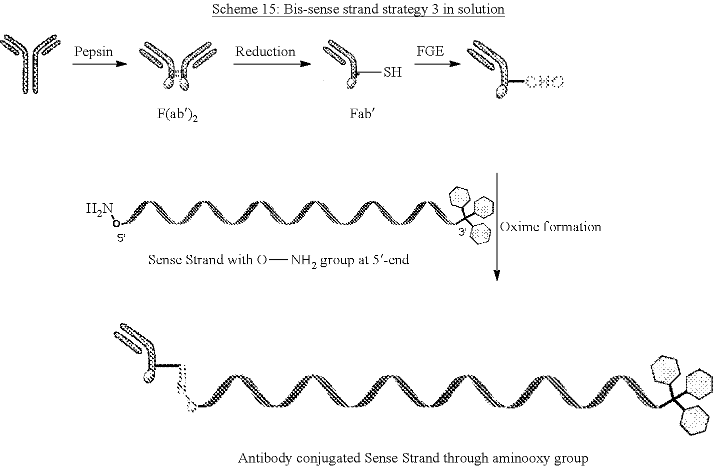

Various exemplary AOCC-conjugate building blocks are summarized in Tables 6-14.

### 5′ Aminooxy-2′-OMe-U Modifications

TABLE 6
Hetero bis-alkyl Modification

| Structure | R₁ ≠ R₂ | |
|---|---|---|
| 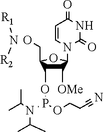 | 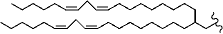 [SMILES: *CC(CCCCCCCC/C=C\C/C=C\CCCCC)CCCCCCCC/C=C\C/C=C\CCCCC] DiLin | 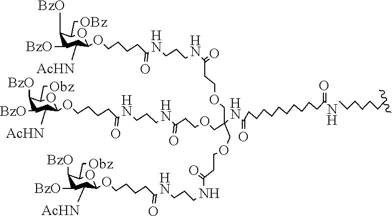 |
| | 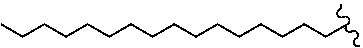 [SMILES: *CCCCCCCCCCCCCCCC] C16 | 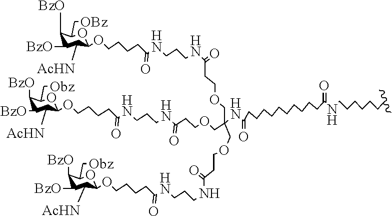 |
| | 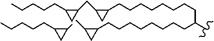 [SMILES: *C(CCCCCCCCC1CC1CC1CC1CCCCC)CCCCCCCCC1CC1CC1CC1CCCCC] | —CH₃ |
| | 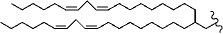 [SMILES: *CC(CCCCCCCC/C=C\C/C=C\CCCCC)CCCCCCCC/C=C\C/C=C\CCCCC] | —CH₃ |
| | 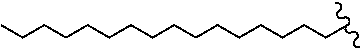 [SMILES: *CCCCCCCCCCCCCCCC] | —CH₃ |
| | 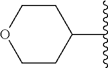 [SMILES: *C1CCOCC1] | —CH₃ |
| | 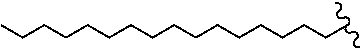 [SMILES: *CCCCCCCCCCCCCCCC] C16 | 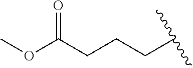 [SMILES: *CCCC(=O)OC] C4-acid |
| | 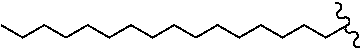 [SMILES: *CCCCCCCCCCCCCCCC] C16 | 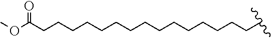 [SMILES: *CCCCCCCCCCCCCCCC(=O)OC] C16-acid |
| | 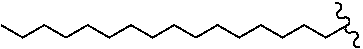 [SMILES: *CCCCCCCCCCCCCCCC] C16 | 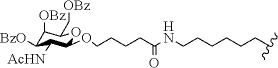 |
| | 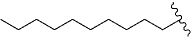 [SMILES: *CCCCCCCCCC] C10 | 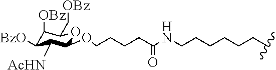 |

TABLE 7
Homo bis alkyl Modification

| Structure | R |
|---|---|
| 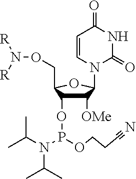 | 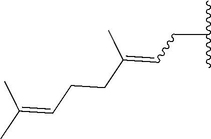 [SMILES: *CC=C(C)CCC=C(C)C] Citral |
| | 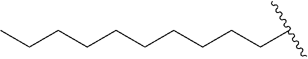 [SMILES: *CCCCCCCCCC] |
| | C10 |
| | 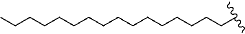 [SMILES: *CCCCCCCCCCCCCCCC] |
| | C16 |
| | 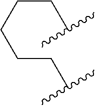 [SMILES: *CCCCC*] |
| | 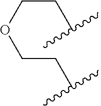 [SMILES: *CCOCC*] |

TABLE 8
Hetero-alkyl-amido Modification

| Structure | R₁ ≠ R₂ | |
|---|---|---|
| 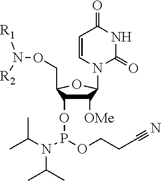 | 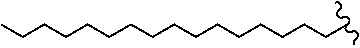 [SMILES: *CCCCCCCCCCCCCCCC] C16 | 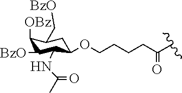 |
| | 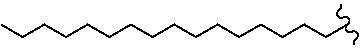 [SMILES: *CCCCCCCCCCCCCCCC] C16 | 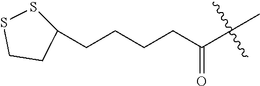 [SMILES: *C(=O)CCCCC1CCSS1] |
| | 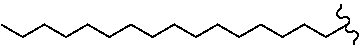 [SMILES: *CCCCCCCCCCCCCCCC] C16 | 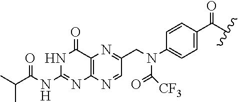 [SMILES: *C(=O)c1ccc(N(Cc2cnc3nc(NC(=O)C(C)C)[nH]c(=O)c3n2)C(=O)C(F)(F)F)cc1] |
| | 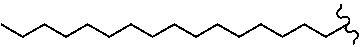 [SMILES: *CCCCCCCCCCCCCCCC] C16 | 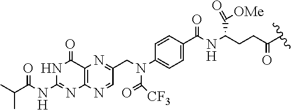 [SMILES: *C(=O)CC[C@H](NC(=O)c1ccc(N(Cc2cnc3nc(NC(=O)C(C)C)[nH]c(=O)c3n2)C(=O)C(F)(F)F)cc1)C(=O)OC] |
| | 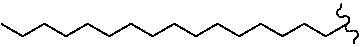 [SMILES: *CCCCCCCCCCCCCCCC] C16 | 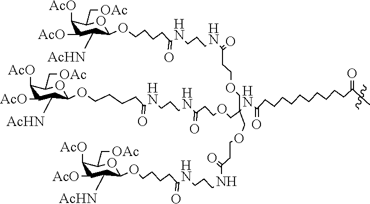 |

TABLE 9
Hetero-Oxime Modification

| Structure | R₁ ≠ R₂ | |
|---|---|---|
| 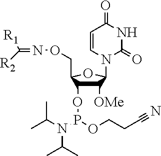 | 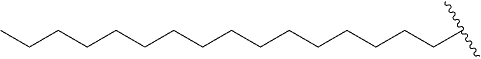 [SMILES: *CCCCCCCCCCCCCCCC] C16 | H |
| | 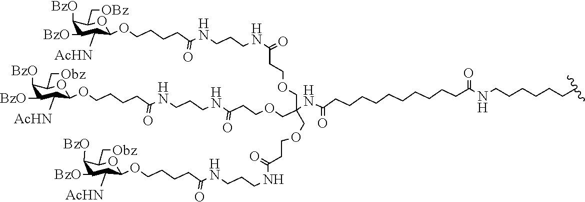 | H |
| | 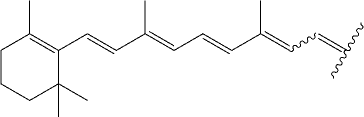 [SMILES: *=CC=C(C)/C=C/C=C(C)/C=C/C1=C(C)CCCC1(C)C] | H |
| | All trans retinal | |

TABLE 10
Homo-Oxime Modification

| Structure | R₁ ≠ R₂ |
|---|---|
| 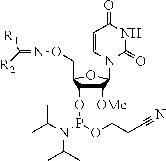 | 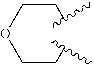 [SMILES: *CCOCC*] 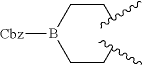 [SMILES: *CCB(CC*)C(=O)OCc1ccccc1] |
| | 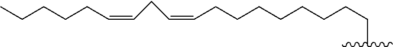 [SMILES: *CCCCCCCC/C=C\C/C=C\CCCCC] |
| | 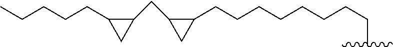 [SMILES: *CCCCCCCCC1CC1CC1CC1CCCCC] |

### 5′-Aminooxy-LNA-A Modifications

TABLE 11
Hetero-bis-alkyl Modification

| Structure | R₁ ≠ R₂ | |
|---|---|---|
| 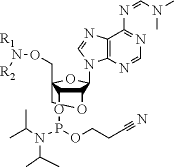 | 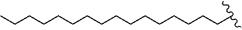 [SMILES: *CCCCCCCCCCCCCCCC] C16 | 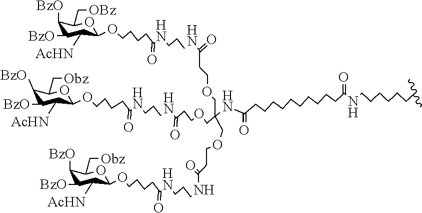 |
| | 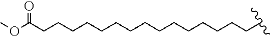 [SMILES: *CCCCCCCCCCCCCCCC(=O)OC] C16 | 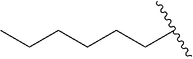 [SMILES: *CCCCCC] |
| | 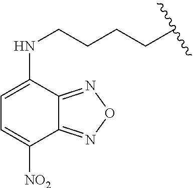 [SMILES: *CCCCNc1ccc([N+](=O)[O-])c2nonc12] | 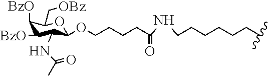 |
| | 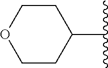 [SMILES: *C1CCOCC1] | 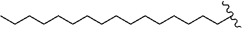 [SMILES: *CCCCCCCCCCCCCCCC] C16 |
| | 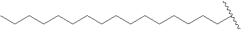 [SMILES: *CCCCCCCCCCCCCCCC] C16 | 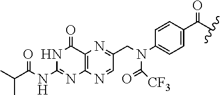 [SMILES: *C(=O)c1ccc(N(Cc2cnc3nc(NC(=O)C(C)C)[nH]c(=O)c3n2)C(=O)C(F)(F)F)cc1] |

TABLE 12
Homo bis-alkyl Modification

| Structure | R₁ ≠ R₂ |
|---|---|
| 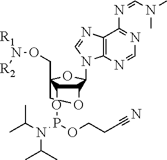 | 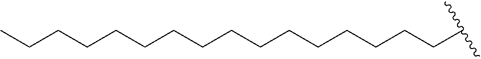 [SMILES: *CCCCCCCCCCCCCCCC] C16 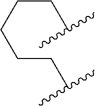 [SMILES: *CCCCC*] |
| | 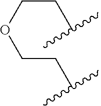 [SMILES: *CCOCC*] |

TABLE 13
Hetero oxime-Modification

| Structure | R₁ ≠ R₂ | |
|---|---|---|
| 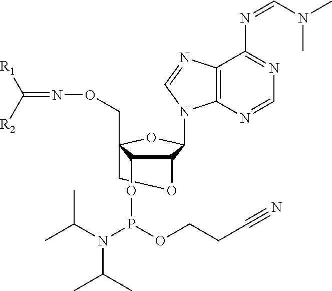 | 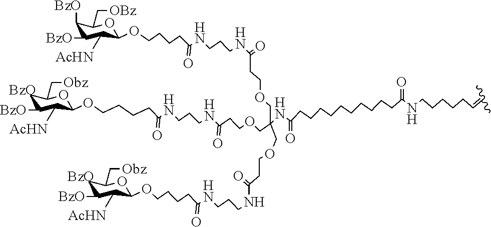 | H |
| | 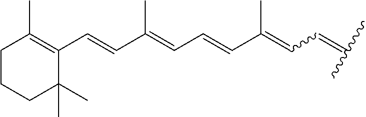 [SMILES: *=CC=C(C)/C=C/C=C(C)/C=C/C1=C(C)CCCC1(C)C] | H |
| | All trans retinal | |

TABLE 14
Homo-Oxime Modification

| Structure | R₁ ≠ R₂ |
|---|---|
| 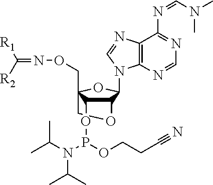 | 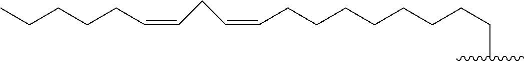 [SMILES: *CCCCCCCC/C=C\C/C=C\CCCCC] |

Stability towards 5′-specific exonucleases: Stability towards 5′-specific exonucleases. Modified ONs were prepared in a final concentration of 0.1 mg/mL in either 50 mM Tris (pH 7.2), 10 mM MgCl₂ or 50 mM sodium acetate (pH 6.5), 10 mM MgCl₂ for 5′-specific exonuclease experiments, respectively. The exonuclease (150 mU/mL SVPDE or 500 mU/mL PDII) was added immediately prior to analysis via IEX HPLC (dionex DNAPac PA200, 4×250 mm) using a gradient of 37-52% mobile phase (1 M NaBr, 20 mM sodium phosphate, pH 11, 15% MeCN; stationary phase: 20 mM sodium phosphate, 15% MeCN, pH 11) over 7.5 min with a flow of 1 mL/min. Samples were analyzed at intervals for 24 h. Absorbance was monitored at 260 nm. The quantity of full-length ON was determined as the area under the curve. Percent full length ON was calculated by dividing by the area under the curve at t=0 and multiplying by 100 Each aliquot of enzyme was thawed just prior to the experiment. The half-life was determined by fitting to first order kinetics.

The advantages of AOCC at 5′-end of RNA strands include, but are not limited to the following:
1. Better strand bias—phosphorylation of sense strand will be blocked (RISC loading in vivo);
2. Potential Elimination of 2PS at the 5′ end of sense strand (improved nuclease resistance)-hindered ligand give exo-nuclease stability to sense strand (in vivo);
3. Multiple ligands conjugation;
4. PK modulation (one targeting ligand, one PK enhancer); and
5. Efficacy (in vitro & in vivo) and improved distribution (in vivo).

It is noted that other conjugation tools along with AOCC²⁹ can be used.

### Synthesis of Building Blocks

### Materials and Methods

General conditions: TLC was performed on Merck silica gel 60 plates coated with F254. Compounds were visualized under UV light (254 nm) or after spraying with the p-anisaldehyde staining solution followed by heating. Flash column chromatography was performed using a Teledyne ISCO Combi Flash system with pre-packed RediSep Teledyne ISCO silica gel cartridges. All moisture-sensitive reactions were carried out under anhydrous conditions using dry glassware, anhydrous solvents, and argon atmosphere. All commercially available reagents and solvents were purchased from Sigma-Aldrich unless otherwise stated and were used as received. ESI-MS spectra were recorded on a Waters QTof Premier instrument using the direct flow injection mode. ¹H NMR spectra were recorded at 500 and 600 MHz. ¹³C NMR spectra were recorded at 126 and 151 MHz. ³¹P NMR spectra were recorded at 202 and 243 MHz. ¹⁹F NMR spectra were recorded at 565 MHz. Chemical shifts are given in ppm referenced to the solvent residual peak (DMSO-d₆—1H: δ at 2.50 ppm and 13C δ at 39.5 ppm; CDCl₃—1H: δ at 7.26 ppm and 13C δ at 77.16 ppm). Coupling constants are given in Hertz. Signal splitting patterns are described as singlet (s), doublet (d), triplet (t), septet (sept), broad signal (brs), or multiplet (m).

5′-aminooxy-2′-OMe Building Blocks

5′-aminooxy-2′-OMe building blocks were synthesized following the method in Scheme 16.

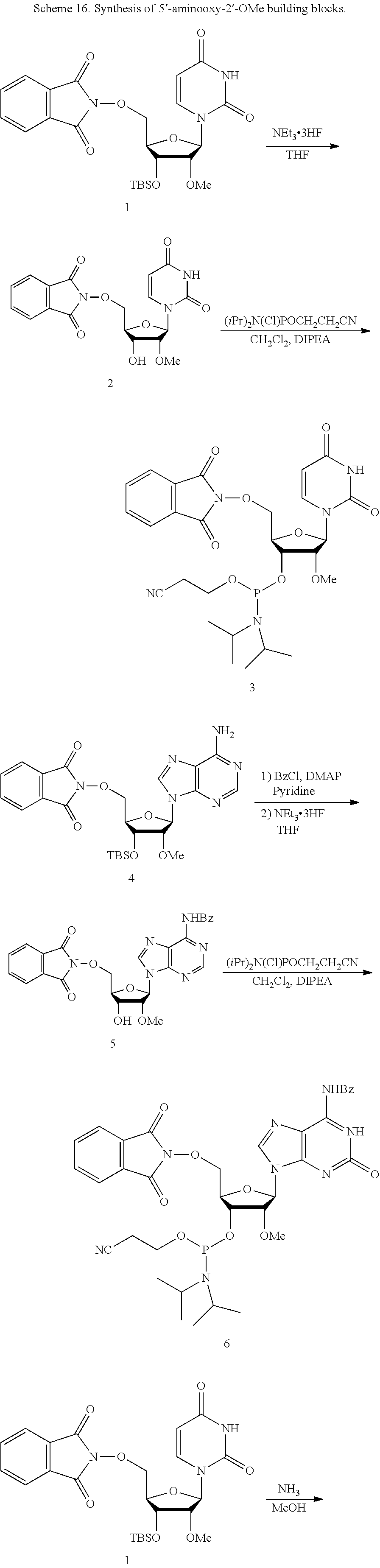

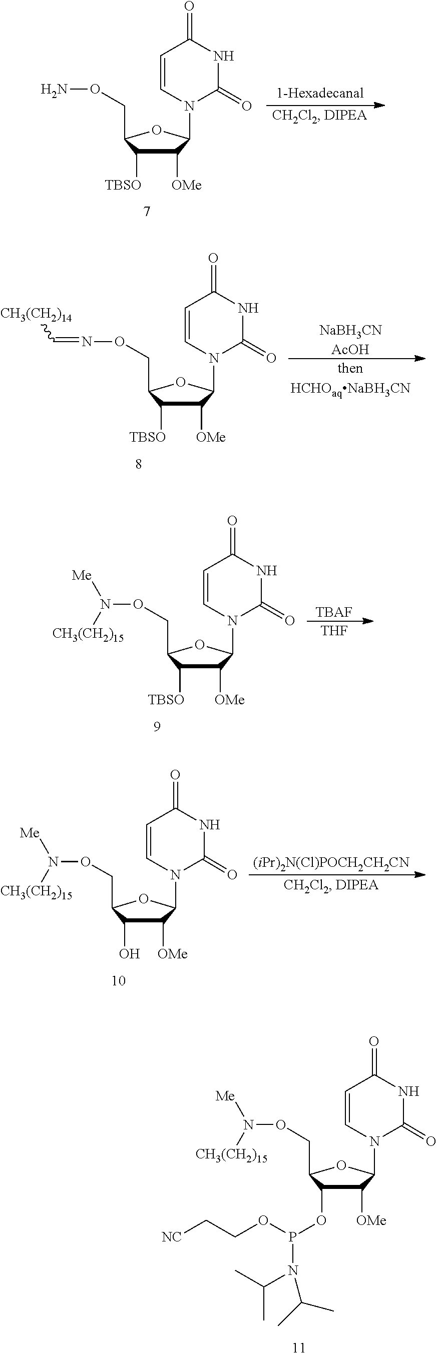

### 2-(((2R,3R,4R,5R)-5-(2,4-dioxo-3,4-dihydropyrimidin-1(2H)-yl)-3-hydroxyl-4-methoxytetra hydrofuran-2-yl)methoxy)isoindoline-1,3-dione Compound 2

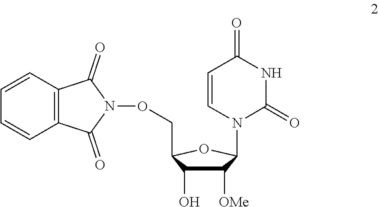

To a solution of compound 1³⁰ (3.05 g, 5.90 mmol) in anhydrous THF (60 mL) was added NEt₃·3HF (3.21 mL, 59.0 mmol of HF) at ambient temperature. The reaction mixture was stirred overnight at 60° C. then diluted with CH₂Cl₂ and washed with saturated aqueous NaHCO₃ solution. The organic layer was separated, dried over anhydrous Na₂SO₄, filtered, and concentrated. The crude material was purified by flash silica gel column chromatography (0-5% MeOH in AcOEt) to give compound 2 (2.07 g, 5.13 mmol, 87%, R_{f}=0.36; developed with 5% MeOH in AcOEt). ¹H NMR (500 MHz, DMSO-d₆): δ 11.37 (s, 1H), 7.86 (s, 4H), 7.77 (d, J=8.0 Hz, 1H), 5.86 (d, J=5.5 Hz, 1H), 5.61 (d, J=8.0 Hz, 1H), 5.42 (d, J=5.5 Hz, 1H), 4.44-4.36 (m, 2H), 4.26-4.20 (m, 1H), 4.17-4.12 (m, 1H), 3.88 (dd, J=5.5, 5.0 Hz, 1H), 3.37 (s, 3H). ¹³C NMR (126 MHz, DMSO-d₆): δ 163.02, 162.96, 150.43, 140.65, 134.81, 128.55, 123.28, 102.13, 86.82, 81.80, 81.61, 77.38, 68.60, 57.64. HRMS calc. for C₁₈H₁₇N₃O₈Na [M+H]⁺ 426.0913, found 426.0918.

### 2-cyanoethyl-((2R,3R,4R,5R)-5-(2,4-dioxo-3,4-dihydropyrimidin-1(2H)-yl)-2-(((1,3-dioxoisoindolin-2-yl)oxy)methyl)-4-methoxytetrahydrofuran-3-yl) diisopropylphosphoramidite Compound 3

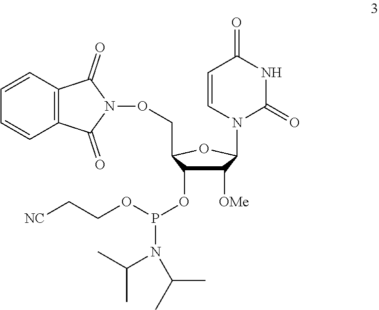

To a solution of compound 2 (2.00 g, 4.96 mmol) in anhydrous CH₂Cl₂(50 mL) and N,N-diisopropylethylamine (2.59 mL, 14.9 mmol) was added 2-cyanoethyl N,N-diisopropylchlorophosphoramidite (1.22 mL, 5.46 mmol). The reaction mixture was stirred at ambient temperature for 4 h under argon atmosphere. The reaction mixture was diluted with CH₂Cl₂(100 mL) then washed with saturated aqueous NaHCO₃. The organic layer was separated, dried over anhydrous Na₂SO₄, filtered, and concentrated. The crude material was purified by flash silica gel column chromatography (50-80% EtOAc in hexanes) to give compound 3 (2.31 g, 3.83 mmol, 70%, R_{f}=0.52 developed with 80% EtOAc in hexanes) as a white foam. ¹H NMR (400 MHz, Acetonitrile-d3): δ 9.13 (brs, 1H), 7.91-7.88 (m, 1H), 7.86-7.80 (m, 5H), 5.94-5.91 (m, 1H), 5.66-5.62 (m, 1H), 5.66-5.62 (m, 1H), 4.63-4.33 (m, 4H), 4.02-3.98 (m, 1H), 3.93-3.60 (m, 4H), 3.50-3.42 (m, 3H), 2.72-2.64 (m, 2H), 1.25-1.14 (m, 12H). ³¹P NMR (202 MHz, Acetonitrile-d3): δ 152.02, 151.59. ¹³C NMR (126 MHz, Acetonitrile-d3) δ 164.41, 164.35, 163.98, 163.95, 151.57, 151.54, 141.40, 141.37, 135.87, 135.86, 130.05, 130.03, 124.32, 119.69, 119.66, 103.20, 103.11, 88.81, 88.39, 83.29, 83.27, 82.79, 82.77, 82.75, 82.37, 82.33, 77.96, 77.80, 72.14, 72.01, 71.37, 71.24, 61.03, 59.98, 59.84, 59.29, 59.15, 59.13, 58.79, 58.77, 44.33, 44.23, 44.12, 25.03, 24.99, 24.96, 24.93, 24.87, 21.22, 21.07, 21.04, 21.02, 20.98, 14.59. HRMS calc. for C₂₇H₃₅N₅O₉P [M+H]⁺ 604.2172, found 604.2182.

### N-(9-((2R,3R,4R,5R)-5-(((1,3-dioxoisoindolin-2-yl)oxy)methyl)-4-hydroxyl-3-methoxytetrahydrofuran-2-yl)-9H-purin-6-yl)benzamide Compound 5

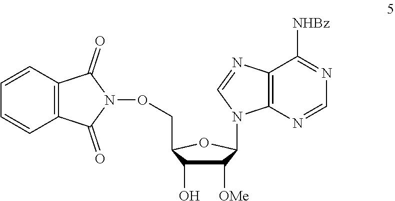

To a solution of compound 4 (3.00 g, 5.55 mmol) in anhydrous Pyridine (56 mL) were added benzoyl chloride (1.93 mL, 16.7 mmol) and DMAP (67.8 mg, 0.555 mmol). The reaction mixture was stirred at ambient temperature for 5 h under argon atmosphere. The reaction mixture was diluted with CH₂Cl₂(100 mL) then washed with saturated aqueous NaHCO₃. The organic layer was separated, dried over anhydrous Na₂SO₄, filtered, and concentrated. The crude material was purified by flash silica gel column chromatography (50-80% EtOAc in hexanes) to give mono- and bis-benzoated compounds separately. (mono-benzoated compound 12 1.43 g, 2.22 mmol, 40%, R_{f}=0.26; developed with hexane:EtOAc=2:1 and bis-benzoated compound 13 2.21 g, 2.95 mmol, 54%, R_{f}=0.34; developed with hexane:EtOAc=1:1). Compound 12: ¹H NMR (400 MHz, DMSO-d₆): δ 11.21 (brs, 1H), 8.74 (s, 1H), 8.04 (d, J=7.6 Hz, 1H), 7.81 (s, 4H), 7.64 (dd, J=7.6, 7.2 Hz, 1H), 7.54 (dd, J=8.0, 7.6 Hz, 1H), 6.17 (d, J=5.6 Hz, 1H), 4.76 (dd, J=8.4, 8.0 Hz, 1H), 4.63 (dd, J=5.2, 4.8 Hz, 1H), 4.57-4.44 (m, 2H), 4.34-4.29 (m, 1H), 3.34 (s, 3H), 0.92 (s, 9H), 0.17 (s, 3H), 0.15 (s, 3H). ¹³C NMR (101 MHz, DMSO-d₆): δ 165.54, 162.90, 151.94, 151.73, 150.42, 143.05, 134.71, 133.29, 132.42, 128.46, 128.42, 128.40, 125.77, 123.17, 85.75, 83.02, 81.39, 76.97, 70.45, 57.72, 25.62, 17.79, −4.89, −4.94. HRMS calc. for C₃₂H₃₇N₆O₇Si [M+H]⁺ 645.2493, found 645.2490.

Compound 13: ¹H NMR (400 MHz, DMSO-d₆): δ 8.89 (s, 1H), 8.70 (s, 1H), 7.83 (s, 4H), 7.78 (d, J=7.6 Hz, 4H), 7.59 (dd, J=8.0, 7.6 Hz, 2H), 7.46 (dd, J=8.0, 7.6 Hz, 4H), 6.19 (d, J=5.6 Hz, 1H), 4.75 (dd, J=4.4, 4.0 Hz, 1H), 4.59 (dd, J=5.2, 4.8 Hz, 1H), 4.54-4.44 (m, 2H), 4.34-4.29 (m, 1H), 3.34 (s, 3H), 0.90 (s, 9H), 0.15 (s, 3H), 0.14 (s, 3H). ¹³C NMR (101 MHz, DMSO-d₆): δ 171.96, 162.95, 152.47, 151.90, 150.95, 145.44, 134.75, 133.40, 133.35, 129.00, 128.97, 128.47, 127.13, 123.23, 85.98, 82.95, 81.53, 76.91, 70.32, 59.69, 57.69, 25.58, 17.75, −4.92, −4.97. HRMS calc. for C₃₉H₄₁N₆O₈Si [M+H]⁺ 749.2755, found 749.2754.

To a solution of compound 12 (1.43 g, 2.22 mmol) and compound 13 (2.21 g, 2.95 mmol) in anhydrous THF (50 mL) was added NEt₃ 3HF (2.83 mL, 52.0 mmol of HF) at ambient temperature. The reaction mixture was stirred overnight at 60° C. then diluted with CH₂Cl₂ and washed with saturated aqueous NaHCO₃ solution. The organic layer was separated, dried over anhydrous Na₂SO₄, filtered, and concentrated. The crude material was purified by flash silica gel column chromatography (0-5% MeOH in AcOEt) to give compound 5 (1.90 g, 3.59 mmol, 70%, R_{f}=0.31; developed with 5% MeOH in AcOEt). ¹H NMR (400 MHz, DMSO-d₆): δ 11.20 (brs, 1H), 8.75-8.71 (m, 2H), 8.05 (d, J=7.2 Hz, 2H), 7.80 (s, 4H), 7.63 (dd, J=7.6, 7.2 Hz, 1H), 7.54 (dd, J=7.6, 7.2 Hz, 2H), 6.19 (d, J=4.4 Hz, 1H), 5.58 (brs, 1H), 5.61 (d, J=8.0 Hz, 1H), 5.42 (d, J=5.5 Hz, 1H), 4.58-4.43 (m, 4H), 4.36-4.31 (m, 1H), 3.38 (s, 3H). ¹³C NMR (126 MHz, DMSO-d₆): δ 165.57, 162.98, 151.99, 151.78, 150.41, 142.94, 134.68, 133.30, 132.43, 128.48, 128.44, 125.71, 123.15, 85.86, 82.81, 81.93, 77.50, 69.04, 57.70. HRMS calc. for C₁₈H₁₈N₃O₈ [M+H]⁺ 531.1628, found 531.1623.

### (2R,3R,4R,5R)-5-(6-benzamido-9H-purin-9-yl)-2-(((1,3-dioxoisoindolin-2-yl)oxy)methyl)-4-methoxytetrahydrofuran-3-yl (2-cyanoethyl) diisopropylphosphoramidite Compound 6

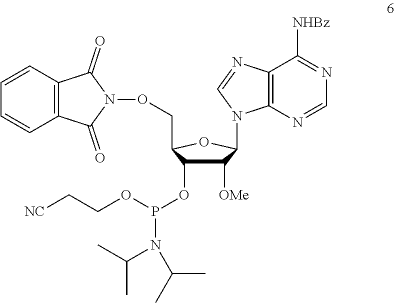

To the solution of compound 5 (1.15 g, 2.17 mmol) in dry DCM 22 mL were added DIPEA (1.10 mL, 6.51 mmol) and 2-cyanoethylchloro-N,N-diisopropylphosphoramidite (581 μL, 2.60 mmol) dropwisely. The reaction mixture was stirred for 3 h at room temperature and then diluted with DCM. Quenched the reaction with saturated aq. NaHCO₃. Organic layer was separated and washed with brine. The solvent was removed in vacuo. The crude residue was purified via column chromatography on silica gel (50-80% EtOAc in hexanes) to afford a colorless form (1.11 g, 70%, R_{f}=0.25; developed with 50% EtOAc in hexane). ¹H NMR (500 MHz, Acetonitrile-d3) δ 9.44 (brs, 1H), 8.63 (s, 1H), 8.52-8.50 (m, 1H), 8.00 (d, J=2.0 Hz, 1H), 7.77 (s, 4H), 7.62 (dd, J=7.5, 7.0 Hz, 1H), 7.52 (dd, J=7.5, 7.0 Hz, 1H), 6.19-6.15 (m, 1H), 4.89-4.80 (m, 1H), 4.65-4.46 (m, 4H), 3.95-3.65 (m, 4H), 3.49-3.42 (m, 3H), 2.77-2.63 (m, 2H), 1.28-1.16 (m, 12H). ¹³C NMR (101 MHz, Acetonitrile-d3) δ 163.00, 162.95, 151.63, 149.58, 142.05, 134.36, 134.34, 133.61, 132.20, 128.55, 128.54, 128.30, 127.80, 124.31, 124.23, 122.82, 118.29, 86.83, 86.49, 82.34, 82.31, 82.06, 81.87, 81.80, 81.74, 76.93, 76.84, 71.48, 71.33, 70.66, 70.50, 59.63, 58.73, 58.55, 57.99, 57.80, 57.53, 57.50, 43.00, 42.88, 42.76, 23.72, 23.67, 23.64, 23.57, 23.51, 19.83, 19.74, 19.70, 19.67, 19.63, 13.21. ³¹P NMR (202 MHz, Acetonitrile-d3) δ 152.18, 151.38. HRMS calc. for C₃₅H₄₁N₈O₈P [M+H]⁺ 731.2707, found 731.2704.

### 1-((2R,3R,4R,5R)-5-((aminooxy)methyl)-4-((tert-butyldimethylsilyl)oxy)-3-methoxytetrahydro-furan-2-yl)pyrimidine-2,4(1H,3H)-dione Compound 7

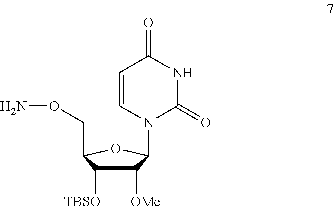

To a solution of compound 1³⁰ (1.00 g, 1.93 mmol) in MeOH (20 mL) was added ammonia solution, 7 N in MeOH (3.00 mL). The reaction mixture was stirred for 1 h at ambient temperature then concentrated. The crude material was purified by flash silica gel column chromatography (0-5% MeOH in AcOEt) to give compound 7 (713 mg, 1.83 mmol, 95%, R_{f}=0.25; developed with 5% MeOH in AcOEt). ¹H NMR (400 MHz, DMSO-d₆): δ 11.37 (brs, 1H), 7.70 (d, J=8.0 Hz, 1H), 6.20 (brs, 1H), 5.79 (d, J=4.8 Hz, 1H), 5.65 (d, J=4.8 Hz, 1H), 4.25 (dd, J=4.8, 4.4 Hz, 1H), 3.96 (dd, J=8.4, 4.8 Hz, 1H), 3.83 (dd, J=5.2, 4.8 Hz, 1H), 3.78-3.65 (m, 2H), 3.32 (s, 3H). ¹³C NMR (101 MHz, DMSO-d₆): δ 162.97, 150.42, 140.47, 102.11, 86.63, 82.41, 81.64, 74.55, 70.21, 57.53, 25.59, 25.54, 17.75, −4.86, −5.01. HRMS calc. for C₁₆H₃₀N₃O₆Si [M+H]⁺ 388.1904, found 388.1909.

Palmitaldehyde-O-(((2R,3R,4R,5R)-3-((tert-butyldimethylsilyl)oxy)-5-(2,4-dioxo-3,4-dihydropyrimidin-1(2H)-yl)-4-methoxytetrahydrofuran-2-yl)methyl) oxime Compound 8:

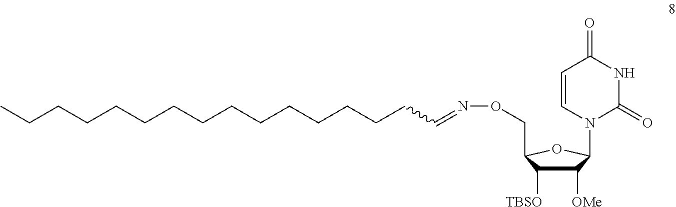

To a solution of compound 7 (2.00 g, 5.16 mmol) in DCM (50 mL) were added 1-hexadecanal (1.30 g, 5.42 mmol) and DIPEA (2.62 mL, 15.4 mmol). The reaction mixture was stirred for 1 h at ambient temperature then concentrated. The crude material was purified by flash silica gel column chromatography (75% hexane in AcOEt) to give compound 8 as ca. 1:1 stereoisomers (2.21 g, 3.63 mmol, 70%, R_{f}=0.3.7; developed with 75% hexane in AcOEt). ¹H NMR (400 MHz, DMSO-d₆): δ 11.45-11.30 (m, 1H), 7.68 (d, J=8.0 Hz, 0.5H), 7.63 (d, J=8.0 Hz, 0.5H), 7.46 (dd, J=6.0, 5.6 Hz, 0.5H), 6.78 (dd, J=6.0, 5.2 Hz, 0.5H), 5.79 (dd, J=4.4, 4.0 Hz, 1H), 5.66 (dd, J=8.0, 2.0 Hz, 0.5H), 5.61 (dd, J=8.0, 2.4 Hz, 0.5H), 4.32-3.85 (m, 5H), 3.35 (s, 1.5H), 3.34 (s, 1.5H), 2.31-2.06 (m, 2H), 1.47-1.35 (m, 2H), 1.31-1.13 (m, 24H), 0.90-0.80 (m, 12H), 0.10-0.04 (m, 6H). ¹³C NMR (101 MHz, DMSO-d₆): δ 163.45, 163.43, 152.98, 152.38, 152.37, 150.86, 150.80, 140.63, 140.43, 102.45, 102.31, 87.23, 87.11, 82.81, 82.57, 81.99, 81.82, 72.39, 72.18, 70.27, 70.13, 58.10, 58.01, 31.74, 29.49, 29.46, 29.41, 29.33, 29.20, 29.16, 29.09, 28.89, 26.27, 26.02, 25.92, 25.80, 22.54, 18.21, 14.37, −4.40, −4.45, −4.63, -4.71. HRMS calc. for C₃₂H₆₀N₃O₆Si [M+H]⁺ 610.4251, found 610.4243.

### 1-((2R,3R,4R,5R)-4-((tert-butyldimethylsilyl)oxy)-5-(((hexadecyl(methyl)amino) oxy)methyl)-3-methoxytetrahydrofuran-2-yl)pyrimidine-2,4(1H,3H)-dione Compound 9

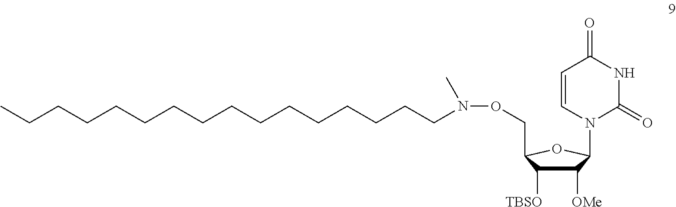

To a solution of compound 8 (2.00 g, 3.28 mmol) in AcOH (8 mL) was added NaCNBH₃ (247 mg, 3.94 mmol) under 15° C. The reaction mixture was stirred for 1 h at 15° C. and to the cold 30% formaldehyde solution (1.64 mL) was added. The stirring was continued for 30 min. and additional amount of NaCNBH₃ (2.47 mg, 3.94 mmol) was added in a similar manner. The resulting mixture was stirred for another 2 hour and then diluted with DCM and washed with ice water. The organic layer was separated and concentrated. The crude material was purified by flash silica gel column chromatography (75% hexane in AcOEt) to give compound 9 (1.89 g, 3.02 mmol, 92%, R_{f}=0.36; developed with 75% hexane in AcOEt). ¹H NMR (400 MHz, DMSO-d₆): δ 11.35 (brs, 1H), 7.74 (d, J=4.4 Hz, 1H), 7.77 (d, J=3.6 Hz, 1H), 5.60 (dd, J=8.0, 2.0 Hz, 1H), 4.14 (dd, J=5.6, 5.2 Hz, 1H), 3.95-3.70 (m, 4H), 3.33 (s, 5H), 2.49 (s, 3H), 1.49-1.38 (m, 2H), 1.31-1.13 (m, 26H), 0.87-0.79 (m, 12H), 0.06 (s, 6H). ¹³C NMR (101 MHz, DMSO-d₆): δ 162.97, 150.26, 140.08, 101.59, 86.82, 81.86, 70.76, 69.90, 60.28, 57.47, 45.22, 31.30, 29.07, 29.04, 29.02, 28.99, 28.97, 28.72, 26.82, 26.60, 25.48, 22.07, 17.65, 13.81, −4.85, −5.26. HRMS calc. for C₃₃H₆₄N₃O₆Si [M+H]⁺ 626.4564, found 626.4568.

### 1-((2R,3R,4R,5R)-5-(((hexadecyl(methyl)amino)oxy)methyl)-4-hydroxyl-3-methoxytetrahydro-furan-2-yl)pyrimidine-2,4(1H,3H)-dione Compound 10

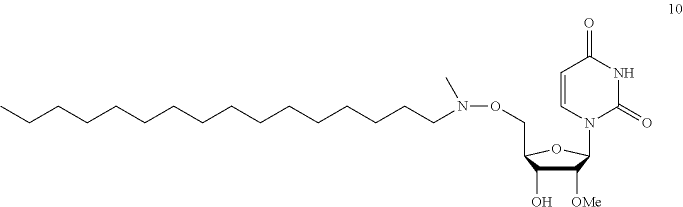

To a solution of compound 9 (1.80 g, 2.88 mmol) in anhydrous THF (30 mL) was added 1M TBAF in THF (5.72 mL) at ambient temperature. The reaction mixture was stirred for 30 min. and then diluted with CH₂Cl₂ and washed with saturated aqueous NH₄Cl solution. The organic layer was separated, dried over anhydrous Na₂SO₄, filtered, and concentrated. The crude material was purified by flash silica gel column chromatography (50% hexane in AcOEt) to give compound 10 (1.06 g, 2.07 mmol, 72%, R_{f}=0.34; developed with 50% hexane in AcOEt). ¹H NMR (500 MHz, DMSO-d₆): δ 11.35 (d, J=2.4 Hz, 1H), 7.73 (d, J=8.0 Hz, 1H), 5.82 (d, J=5.2 Hz, 1H), 5.62 (dd, J=8.0, 2.4 Hz, 1H), 5.23 (brs, 1H), 4.06-4.00 (m, 1H), 3.98-3.92 (m, 1H), 3.86-3.69 (m, 3H), 3.35 (s, 3H), 2.61-2.54 (m, 2H), 2.50 (s, 3H), 1.45 (dq, J=7.2, 6.8 Hz, 2H), 1.31-1.14 (m, 26H), 0.84 (t, J=6.8 Hz, 3H). ¹³C NMR (101 MHz, DMSO-d₆): δ 162.92, 150.38, 140.21, 101.74, 86.28, 82.26, 82.02, 71.40, 68.67, 60.30, 57.49, 45.31, 31.26, 29.00, 28.97, 28.95, 28.67, 26.82, 26.54, 22.06, 13.89. HRMS calc. for C₂₇H₅₀N₃O₆[M+H]⁺ 512.3700, found 512.3694.

### 2-cyanoethyl-((2R,3R,4R,5R)-5-(2,4-dioxo-3,4-dihydropyrimidin-1(2H)-yl)-2-(((hexadecyl(methyl)amino)oxy)methyl)-4-methoxytetrahydrofuran-3-yl)diisopropyl phosphoramidite Compound 11

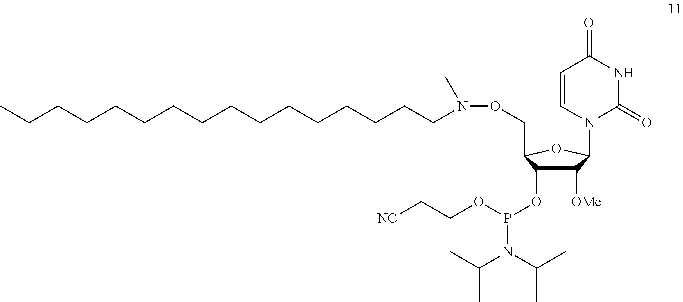

To a solution of compound 10 (1.06 g, 2.07 mmol) in anhydrous DCM (20 mL) were added DIPEA (1.05 mL, 6.21 mmol) and 2-cyanoethylchloro-N,N-diisopropylphosphoramidite (509 μL, 2.28 mmol) dropwisely. The reaction mixture was stirred for 3 h at room temperature and then diluted with DCM. Quenched the reaction with saturated aq. NaHCO₃. Organic layer was separated and washed with brine. The solvent was removed in vacuo. The crude residue was purified via column chromatography on silica gel (20% EtOAc in hexanes) to afford a colorless gum (1.26 g, 86%, R_{f}=0.35; developed with 20% EtOAc in hexane). ¹H NMR (400 MHz, CDCl₃) δ 8.97 (s, 1H), 7.92 (dd, J=15.3, 8.2 Hz, 1H), 5.97 (d, J=3.8 Hz, 1H), 5.68 (dd, J=8.2, 1.2 Hz, 1H), 4.34-4.17 (m, 2H), 4.07 (ddd, J=13.1, 10.9, 2.2 Hz, 1H), 3.96-3.69 (m, 4H), 3.64 (dqd, J=13.6, 6.9, 3.5 Hz, 2H), 3.51 (d, J=11.6 Hz, 3H), 2.73-2.57 (m, 6H), 1.52 (p, J=7.4 Hz, 2H), 1.24 (s, 23H), 1.19 (ddd, J=8.5, 6.7, 2.2 Hz, 12H), 0.87 (t, J=6.7 Hz, 3H) ppm. ¹³C NMR (101 MHz, CDCl₃) δ 163.33, 163.30, 163.27, 150.35, 150.31, 140.25, 140.13, 117.79, 117.58, 102.07, 102.00, 87.82, 87.59, 83.49, 83.46, 83.06, 83.01, 82.36, 82.33, 82.20, 82.15, 77.48, 76.84, 70.63, 70.55, 70.22, 70.06, 61.37, 61.34, 58.89, 58.80, 58.77, 58.72, 58.36, 58.34, 58.20, 58.01, 45.78, 45.74, 43.55, 43.50, 43.43, 43.37, 32.05, 29.83, 29.79, 29.76, 29.74, 29.71, 29.49, 27.61, 27.57, 27.39, 24.82, 24.78, 24.76, 24.75, 24.72, 24.69, 22.82, 20.58, 20.55, 20.51, 20.49, 14.25 ppm. 31p NMR (162 MHz, CDCl₃) δ 151.56, 151.39 ppm.

### Compounds for Post-Synthetic AOCC

2-cyanoethyl-((2R,3R,4R,5R)-5-(2,4-dioxo-3,4-dihydropyrimidin-1(2H)-yl)-2-(((1,3-dioxoisoindolin-2-yl)oxy)methyl)-4-methoxytetrahydrofuran-3-yl) diisopropylphosphoramidite Compound 3:

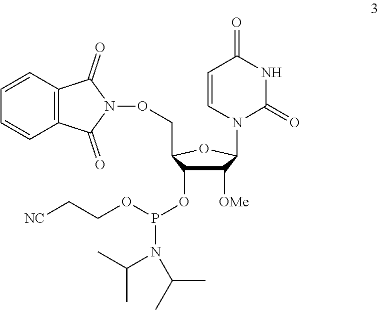

To a solution of compound 2 (2.00 g, 4.96 mmol) in anhydrous CH₂Cl₂ (50 mL) and N,N-diisopropylethylamine (2.59 mL, 14.9 mmol) was added 2-cyanoethyl-N,N-diisopropylchlorophosphoramidite (1.22 mL, 5.46 mmol). The reaction mixture was stirred at ambient temperature for 4 h under argon atmosphere. The reaction mixture was diluted with CH₂Cl₂ (100 mL) then washed with saturated aqueous NaHCO₃. The organic layer was separated, dried over anhydrous Na₂SO₄, filtered, and concentrated. The crude material was purified by flash silica gel column chromatography (50-80% EtOAc in hexanes) to give compound 3 (2.31 g, 3.83 mmol, 70%, R_{f}=0.52 developed with 80% EtOAc in hexanes) as a white foam. ¹H NMR (400 MHz, Acetonitrile-d3): δ 9.13 (brs, 1H), 7.91-7.88 (m, 1H), 7.86-7.80 (m, 5H), 5.94-5.91 (m, 1H), 5.66-5.62 (m, 1H), 5.66-5.62 (m, 1H), 4.63-4.33 (m, 4H), 4.02-3.98 (m, 1H), 3.93-3.60 (m, 4H), 3.50-3.42 (m, 3H), 2.72-2.64 (m, 2H), 1.25-1.14 (m, 12H). ³¹P NMR (202 MHz, Acetonitrile-d3): δ 152.02, 151.59. ¹³C NMR (126 MHz, Acetonitrile-d3) δ 164.41, 164.35, 163.98, 163.95, 151.57, 151.54, 141.40, 141.37, 135.87, 135.86, 130.05, 130.03, 124.32, 119.69, 119.66, 103.20, 103.11, 88.81, 88.39, 83.29, 83.27, 82.79, 82.77, 82.75, 82.37, 82.33, 77.96, 77.80, 72.14, 72.01, 71.37, 71.24, 61.03, 59.98, 59.84, 59.29, 59.15, 59.13, 58.79, 58.77, 44.33, 44.23, 44.12, 25.03, 24.99, 24.96, 24.93, 24.87, 21.22, 21.07, 21.04, 21.02, 20.98, 14.59. HRMS calc. for C₂₇H₃₅N₅O₉P [M+H]⁺ 604.2172, found 604.2182.

### (2R,3R,4R,5R)-5-(6-benzamido-9H-purin-9-yl)-2-(((1,3-dioxoisoindolin-2-yl)oxy) methyl)-4-methoxytetrahydrofuran-3-yl-(2-cyanoethyl)diisopropylphosphoramidite Compound 6

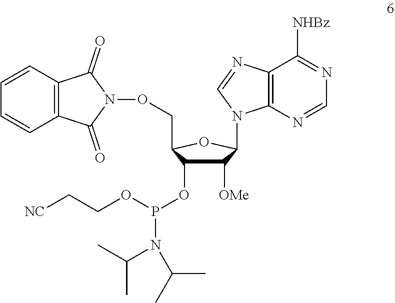

To the solution of compound 5 (1.15 g, 2.17 mmol) in dry DCM 22 mL were added DIPEA (1.10 mL, 6.51 mmol) and 2-cyanoethylchloro-N,N-diisopropylphosphoramidite (581 μL, 2.60 mmol) dropwisely. The reaction mixture was stirred for 3 h at room temperature and then diluted with DCM. Quenched the reaction with saturated aq. NaHCO₃. Organic layer was separated and washed with brine. The solvent was removed in vacuo. The crude residue was purified via column chromatography on silica gel (50-80% EtOAc in hexanes) to afford a colorless form (1.11 g, 70%, R_{f}=0.25; developed with 50% EtOAc in hexane). ¹H NMR (500 MHz, Acetonitrile-d3) δ 9.44 (brs, 1H), 8.63 (s, 1H), 8.52-8.50 (m, 1H), 8.00 (d, J=2.0 Hz, 1H), 7.77 (s, 4H), 7.62 (dd, J=7.5, 7.0 Hz, 1H), 7.52 (dd, J=7.5, 7.0 Hz, 1H), 6.19-6.15 (m, 1H), 4.89-4.80 (m, 1H), 4.65-4.46 (m, 4H), 3.95-3.65 (m, 4H), 3.49-3.42 (m, 3H), 2.77-2.63 (m, 2H), 1.28-1.16 (m, 12H). ¹³C NMR (101 MHz, Acetonitrile-d3) δ 163.00, 162.95, 151.63, 149.58, 142.05, 134.36, 134.34, 133.61, 132.20, 128.55, 128.54, 128.30, 127.80, 124.31, 124.23, 122.82, 118.29, 86.83, 86.49, 82.34, 82.31, 82.06, 81.87, 81.80, 81.74, 76.93, 76.84, 71.48, 71.33, 70.66, 70.50, 59.63, 58.73, 58.55, 57.99, 57.80, 57.53, 57.50, 43.00, 42.88, 42.76, 23.72, 23.67, 23.64, 23.57, 23.51, 19.83, 19.74, 19.70, 19.67, 19.63, 13.21. ³¹P NMR (202 MHz, Acetonitrile-d3) δ 152.18, 151.38. HRMS calc. for C₃₅H₄₁N₈O₈P [M+H]⁺ 731.2707, found 731.2704.

### 2-(8-bromooctoxy)isoindoline-1,3-dione ELN0132-439

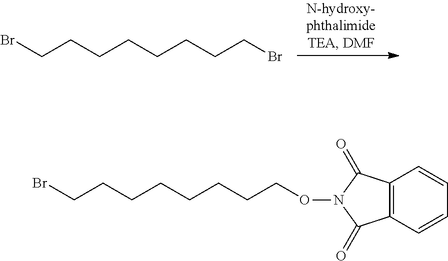 [SMILES: BrCCCCCCCCBr ; O=C1c2ccccc2C(=O)N1OCCCCCCCCBr]

To a clear solution of 1,8-dibromooctane (14.0 g, 50.44 mmol, 9.46 mL) in dry dimethylformamide (30 mL), was added N-hydroxylphathalimide (4.24 g, 25.22 mmol, 97% purity) and triethylamine (3.09 g, 30.26 mmol, 4.26 mL) in single portions. Reaction mixture was stirred for 24 hr. The reaction mixture was filtered and to the filtrated was added EtOAc (100 mL) and water (100 mL) and stirred. Organic layer was separated, washed with brine (4×100 mL), dried over anhydrous Na₂SO₄, filtered and the filtrate was evaporated to dryness. Crude mass obtained, was purified by flash column chromatography (gradient: 0-30% EtOAc in hexane) to afford ELN0132-439 (7.37 g, 41% yield) as transparent gum which slowly turned white solid. ¹H NMR (400 MHz, CDCl₃) δ 7.84 (dd, J=5.5, 3.0 Hz, 2H), 7.79-7.63 (m, 2H), 4.20 (t, J=6.7 Hz, 2H), 3.41 (t, J=6.9 Hz, 2H), 1.94-1.73 (m, 4H), 1.61-1.32 (m, 8H) ppm. ¹³C NMR (101 MHz, CDCl₃) δ 163.79, 134.56, 129.08, 123.60, 78.64, 34.12, 32.89, 29.20, 28.72, 28.22, 28.19, 25.57 ppm.

### 1-((3aR,4R,6R,6aR)-2,2-dibutyl-6-(hydroxylmethyl)tetrahydrofuro[3,4-d][1,3,2]dioxastannol-4-yl)pyrimidine-2,4(1H,3H)-dione ELN0132-142³¹

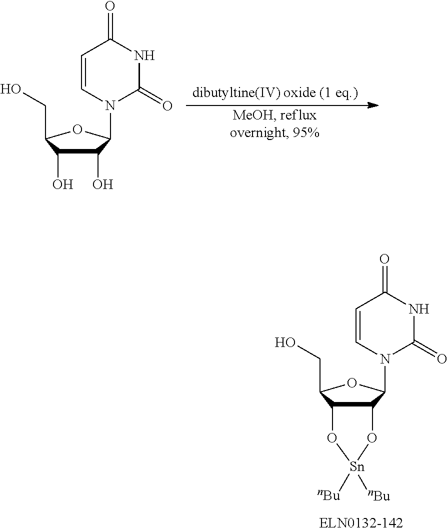

Uridine (15 g, 61.4 mmol) was treated with dibutyltin (IV) oxide (15.29 g, 61.4 mmol) in MeOH (600 mL) to form a suspension and heated under reflux overnight. The resulting cloudy mixture was concentrated in vacuo and placed under high vac. to obtain ELN0132-142 as a white powder (27.7 g). ¹H NMR (500 MHz, DMSO-d₆) δ 7.81 (dd, J=8.1, 1.6 Hz, 1H), 5.02-4.95 (m, 1H), 4.10 (q, J=5.7 Hz, 1H), 4.03 (dd, J=6.0, 3.8 Hz, 1H), 3.68-3.52 (m, 3H), 1.59-1.52 (m, 4H), 1.29 (h, J=7.3 Hz, 4H), 1.18-1.14 (m, 4H), 0.86 (t, J=7.3 Hz, 6H) ppm. ¹³C NMR (151 MHz, DMSO-d₆) δ 163.05, 150.78, 141.09, 101.91, 89.36, 86.49, 77.22, 73.71, 61.73, 26.93, 26.19, 13.56 ppm.

### 2-((8-(((2R,3R,4R,5R)-2-(2,4-dioxo-3,4-dihydropyrimidin-1(2H)-yl)-4-hydroxyl-5-(hydroxylmethyl)tetrahydrofuran-3-yl)oxy)octyl)oxy)isoindoline-1,3-dione (2′-isomer) and 2-((8-(((2R,3S,4R,5R)-5-(2,4-dioxo-3,4-dihydropyrimidin-1(2H)-yl)-4-hydroxyl-2-(hydroxylmethyl) tetrahydrofuran-3-yl)oxy)octyl)oxy)isoindoline-1,3-dione (3′-isomer) ELN0132-388

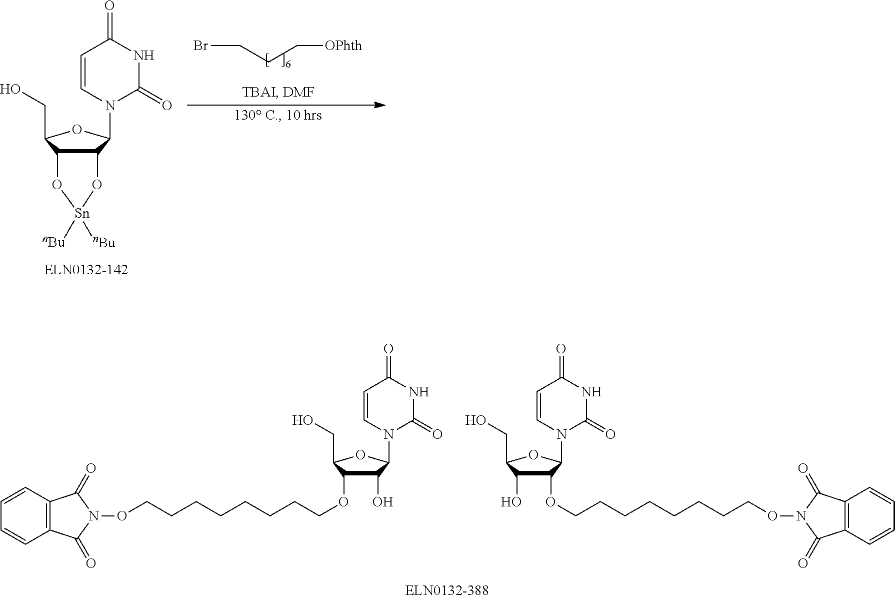

To a solution of ELN0132-142 (3.7 g, 7.79 mmol) in dimethylformamide (60 mL) was added tetrabutylammonium iodide (5.87 g, 15.57 mmol) and ELN0132-439 (6.62 g, 18.69 mmol) in single portions. The resulting mixture was heated to reflux at 130° C. for 10 hr. DMF of the resulting red colored solution was removed under high vacuum to obtain a gummy brown mass which was purified by flash column chromatography (gradient: 0-10% MeOH in DCM) to afford a mixture of 2′- and 3′-isomers of ELN0132-388 (2.35 g, 4.54 mmol, 58.31% yield) as brownish white foam. ¹H NMR (600 MHz, DMSO-d₆) δ 11.32 (dd, J=9.3, 2.2 Hz, 2H), 7.94 (d, J=8.1 Hz, 1H), 7.85 (s, 7H), 5.84 (d, J=5.2 Hz, 1H), 5.75 (d, J=5.4 Hz, 1H), 5.64 (ddd, J=8.0, 6.8, 2.2 Hz, 2H), 5.31 (d, J=6.1 Hz, 1H), 5.13 (td, J=5.1, 3.1 Hz, 2H), 5.04 (d, J=5.8 Hz, 1H), 4.19-4.06 (m, 5H), 3.92 (q, J=3.5 Hz, 1H), 3.88-3.83 (m, 2H), 3.76 (t, J=4.6 Hz, 1H), 3.68-3.60 (m, 1H), 3.60-3.50 (m, 3H), 3.44 (ddt, J=13.0, 9.2, 6.7 Hz, 2H), 1.66 (h, J=7.0 Hz, 3H), 1.60-1.50 (m, 7H), 1.50-1.35 (m, 3H), 1.30 (ddt, J=17.7, 10.6, 5.0 Hz, 17H) ppm. ¹³C NMR (151 MHz, DMSO-d₆) δ 163.28, 163.09, 163.05, 150.73, 150.53, 140.57, 140.39, 134.73, 128.59, 123.19, 101.80, 101.68, 88.00, 86.05, 85.18, 82.80, 81.07, 77.66, 77.46, 72.63, 69.76, 69.61, 68.35, 60.76, 60.52, 57.54, 57.52, 57.51, 29.32, 29.03, 28.78, 28.70, 28.65, 28.64, 27.66, 27.65, 25.47, 25.30, 25.09, 25.07, 23.07, 19.21, 19.20, 13.49 ppm. HRMS calc. for C₂₅H₃₁N₃O₉Na [M+Na]⁺ 540.1958, found 540.1970.

### 2-((8-(((2R,3R,4R,5R)-5-((bis(4-methoxyphenyl)(phenyl)methoxy)methyl)-2-(2,4-dioxo-3,4-dihydropyrimidin-1(2H)-yl)-4-hydroxyltetrahydrofuran-3-yl)oxy)octyl)oxy)isoindoline-1,3-dione ELN0132-395U (2′-isomer) and 2-((8-(((2R,3S,4R,5R)-2-((bis(4-methoxyphenyl)(phenyl)methoxy)methyl)-5-(2,4-dioxo-3,4-dihydropyrimidin-1(2H)-yl)-4-hydroxyltetrahydrofuran-3-yl)oxy)octyl)oxy) isoindoline-1,3-dione ELN0132-395L (3′-isomer)

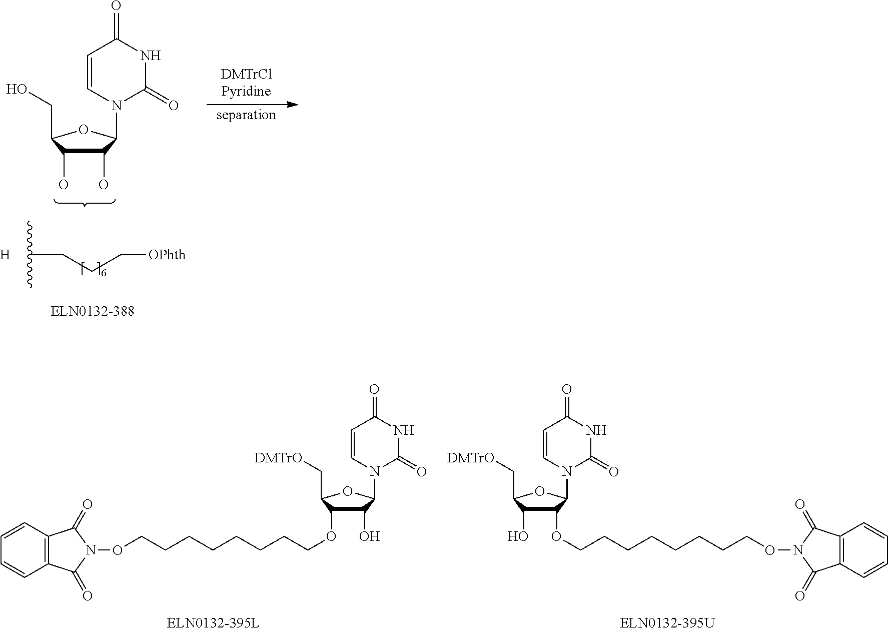

To a clear solution of ELN0132-388 (1.8 g, 3.48 mmol) (mixture of 2′- and 3′-isomers) in pyridine (30 mL) was added 4,4′-dimethoxytrityl chloride (1.41 g, 4.17 mmol) in two portions. Reaction mixture was stirred at for 16 hrs, diluted with DCM (20 mL) and then quenched with 10% NaHCO₃ (20 mL). Organic layer was washed with brine (2×20 mL), separated, dried over anhydrous Na₂SO₄ and filtered. Filtrate was evaporated under high vacuum pump and crude mass obtained, was purified by flash column chromatography (gradient: 20-70% EtOAc in hexane) to afford 2′-isomer (1.04 g) and 3′-isomer (0.67 g) as white foam (combined yield 60%).

2-((8-(((2R,3R,4R,5R)-5-((bis(4-methoxyphenyl)(phenyl)methoxy)methyl)-2-(2,4-dioxo-3,4-dihydropyrimidin-1(2H)-yl)-4-hydroxyltetrahydrofuran-3-yl)oxy)octyl)oxy)isoindo-line-1, 3-dione [ELN0132-395U (2′-isomer)]: ¹H NMR (600 MHz, DMSO) δ 11.40 (d, J=2.2 Hz, 1H), 7.85 (s, 4H), 7.74 (d, J=8.1 Hz, 1H), 7.40-7.35 (m, 2H), 7.32 (t, J=7.7 Hz, 2H), 7.28-7.20 (m, 5H), 6.93-6.88 (m, 4H), 5.80 (d, J=3.8 Hz, 1H), 5.29 (dd, J=8.1, 2.2 Hz, 1H), 5.15 (s, 1H), 4.17 (t, J=5.7 Hz, 1H), 4.11 (t, J=6.5 Hz, 2H), 3.97 (ddd, J=6.8, 4.3, 2.8 Hz, 1H), 3.90 (t, J=4.5 Hz, 1H), 3.74 (s, 6H), 3.63-3.50 (m, 2H), 3.30 (dd, J=10.8, 4.5 Hz, 1H), 3.22 (dd, J=10.8, 2.7 Hz, 1H), 1.69-1.61 (m, 2H), 1.52 (p, J=6.6 Hz, 2H), 1.40 (p, J=7.1 Hz, 2H), 1.29 (dd, J=12.1, 4.7 Hz, 6H) ppm. ¹³C NMR (151 MHz, DMSO) δ 163.34, 163.04, 158.17, 150.34, 144.69, 140.26, 135.38, 135.07, 134.77, 129.82, 128.64, 127.97, 127.73, 126.85, 123.23, 113.30, 113.28, 101.52, 87.02, 85.93, 82.74, 80.86, 77.69, 69.82, 68.48, 62.69, 59.81, 55.08, 29.05, 28.78, 28.71, 27.70, 25.38, 25.13 ppm. HRMS calc. for C₄₆H₄₉N₃O₁₁Na [M+Na]⁺ 842.3265, found 842.3249.

2-((8-(((2R,3S,4R,5R)-2-((bis(4-methoxyphenyl)(phenyl)methoxy)methyl)-5-(2,4-dioxo-3,4-dihydropyrimidin-1(2H)-yl)-4-hydroxyltetrahydrofuran-3-yl)oxy)octyl)oxy)isoindo-line-1, 3-dione [ELN0132-395L (3′-isomer)]: ¹H NMR (600 MHz, DMSO) δ 11.38 (d, J=2.2 Hz, 1H), 7.85 (s, 4H), 7.79 (d, J=8.1 Hz, 1H), 7.40-7.30 (m, 4H), 7.28-7.21 (m, 5H), 6.93-6.87 (m, 4H), 5.70 (d, J=3.5 Hz, 1H), 5.45 (s, 1H), 5.30 (dd, J=8.1, 2.2 Hz, 1H), 4.25 (t, J=4.2 Hz, 1H), 4.11 (t, J=6.5 Hz, 2H), 3.99 (dt, J=6.9, 3.5 Hz, 1H), 3.93 (dd, J=6.5, 4.9 Hz, 1H), 3.74 (s, 6H), 3.59 (dt, J=9.4, 6.4 Hz, 1H), 3.37 (dt, J=9.3, 6.7 Hz, 2H), 3.30 (dd, J=10.9, 3.1 Hz, 1H), 3.24 (dd, J=10.9, 4.0 Hz, 1H), 1.68-1.61 (m, 2H), 1.52-1.45 (m, 2H), 1.39 (q, J=7.1 Hz, 2H), 1.27 (q, J=8.6 Hz, 6H) ppm. ¹³C NMR (151 MHz, DMSO) δ 163.34, 163.11, 158.17, 150.47, 144.68, 140.52, 135.32, 135.15, 134.78, 129.79, 129.77, 128.63, 127.95, 127.69, 126.86, 123.23, 113.27, 101.38, 89.47, 85.96, 80.37, 77.67, 76.72, 72.03, 69.68, 62.35, 59.81, 55.07, 29.22, 28.82, 28.67, 27.70, 25.47, 25.14 ppm. HRMS calc. for C₄₆H₄₉N₃₀₁₁Na [M+Na]⁺ 842.3265, found 842.3251.

3-[[(2R,5R)-2-[[bis(4-methoxyphenyl)-phenyl-methoxy]methyl]-4-[8-(1,3-dioxoisoindolin-2-yl)oxyoctoxy]-5-(2,4-dioxopyrimidin-1-yl)tetrahydrofuran-3-yl]oxy-(diisopropylamino)phos-phanyl]oxypropanenitrile ELN0132-405:

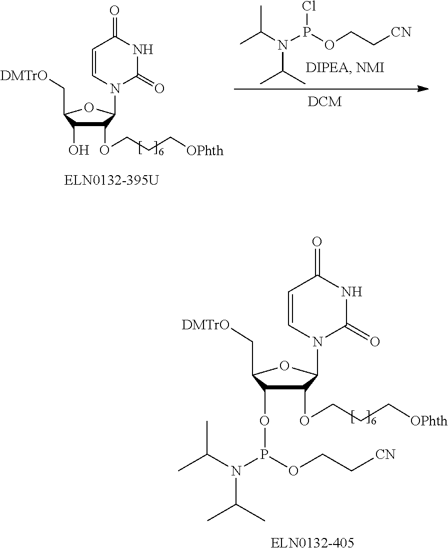

To a clear solution of ELN0132-395U (1.0 g, 1.22 mmol) in DCM (20 mL) was added N-methylimidazole (150.20 mg, 1.83 mmol, 145.83 μL) and diisopropylethylamine (788.15 mg, 6.10 mmol, 1.06 mL) in single portions. After stirring the reaction mixture for 5 minutes at 22° C., 2-cyanoethyl-N,N-diisopropylchlorophosphoramidite (577.34 mg, 2.44 mmol, 544.66 μL) was added and continued stirring for 1 hr and TLC was checked. Starting material was consumed and reaction mixture was diluted with DCM (15 mL). DCM layer was washed with 10% NaHCO₃ (2×25 mL) solution, and brine (30 mL). Organic layer was separated, dried over anhydrous Na₂SO₄, filtered and filtrate was evaporated at 36° C. to afford crude compound which was purified by flash chromatography (20-50% EtOAc in hexane) to afford ELN0132-405 (1.05 g, 84% yield) as white foam. ¹H NMR (600 MHz, CD₃CN) δ 9.15 (s, 1H), 7.97-7.67 (m, 4H), 7.47-7.40 (m, 2H), 7.36-7.21 (m, 7H), 6.91-6.83 (m, 4H), 5.85 (dd, J=11.7, 3.3 Hz, 1H), 5.25-5.19 (m, 1H), 4.54-4.34 (m, 1H), 4.19-4.12 (m, 3H), 4.09-4.00 (m, 2H), 3.91-3.71 (m, 7H), 3.70-3.56 (m, 3H), 3.44-3.31 (m, 2H), 2.67 (td, J=6.8, 5.1 Hz, 1H), 2.56-2.49 (m, 1H), 1.70 (dqd, J=9.7, 6.6, 3.2 Hz, 2H), 1.56 (dq, J=13.7, 6.6 Hz, 2H), 1.44 (qt, J=8.1, 4.7 Hz, 2H), 1.34 (dd, J=13.6, 7.2 Hz, 6H), 1.23-1.12 (m, 10H), 1.04 (dd, J=6.8, 2.6 Hz, 2H) ppm. ¹³C NMR (151 MHz, CD3CN) δ 164.64, 163.94, 163.90, 159.72, 159.71, 159.69, 151.26, 145.73, 145.66, 140.99, 140.96, 136.39, 136.35, 136.25, 136.17, 135.52, 131.17, 131.10, 130.05, 129.03, 128.99, 128.92, 127.99, 123.97, 119.59, 119.44, 114.09, 114.07, 102.53, 102.43, 88.71, 88.59, 87.61, 87.58, 83.39, 83.37, 83.27, 83.23, 82.32, 81.64, 81.61, 79.05, 71.61, 71.37, 71.15, 71.05, 70.99, 70.91, 62.90, 62.42, 60.93, 59.58, 59.46, 59.20, 59.06, 55.89, 55.87, 43.98, 43.93, 43.90, 43.85, 30.34, 30.32, 29.98, 29.94, 29.91, 29.86, 28.85, 26.64, 26.62, 26.27, 26.23, 25.07, 25.02, 24.97, 24.92, 24.88, 24.86, 24.81, 21.13, 21.06, 21.02, 21.00 ppm. ³¹P NMR (243 MHz, CD₃CN) δ 149.54, 149.11 ppm. HRMS calc. for C₅₅H₆₇N₅O₁₂P [M+H]⁺ 1020.4524, found 1020.4531.

### 4-[(2R,5R)-2-[[bis(4-methoxyphenyl)-phenyl-methoxy]methyl]-4-[8-(1,3-dioxoisoindolin-2-yl)oxyoctoxy]-5-(2,4-dioxopyrimidin-1-yl)tetrahydrofuran-3-yl]oxy-4-oxo-butanoic acid ELN0132-472

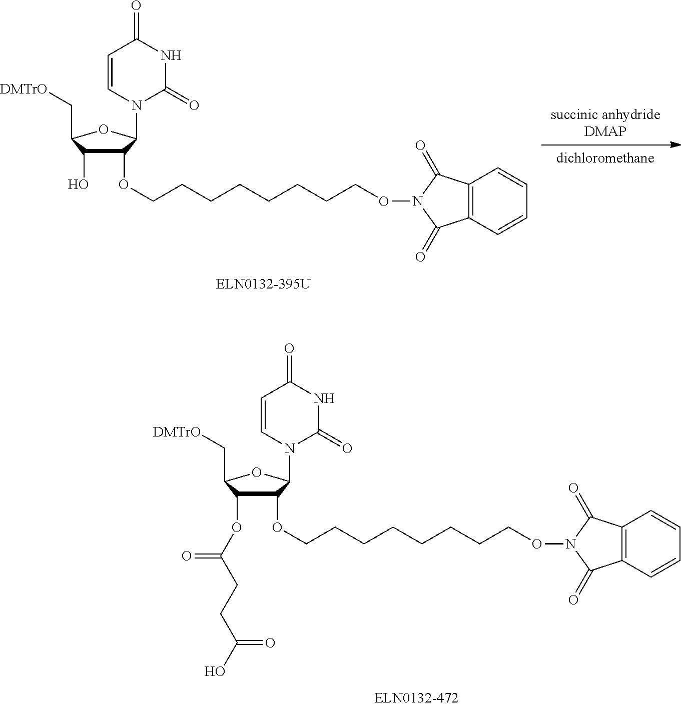

To a clear solution of ELN0132-395U (0.46 g, 561.05 mol) in dichloromethane (10 mL) was added DMAP (274.17 mg, 2.24 mmol) and succinic anhydride (168.43 mg, 1.68 mmol) sequentially. The resulting mixture was stirred for 3 hr at 22° C. Rection mixture was diluted with DCM (15 mL) and organic layer was washed with 10% NH₄Cl solution (2×20 mL). Organic layer was separated, dried over anhydrous Na₂SO₄, filtered and the filtrate was evaporated to dryness. The crude mass thus obtained was purified by flash column chromatography (gradient: 0-5% Methanol in DCM) for afford ELN0132-472 (0.42 g, 81% yield) as white foam. ¹H NMR (600 MHz, DMSO) δ 12.24 (s, 1H), 11.45 (d, J=2.2 Hz, 1H), 7.85 (s, 4H), 7.71 (d, J=8.1 Hz, 1H), 7.39-7.29 (m, 4H), 7.28-7.20 (m, 5H), 6.95-6.88 (m, 4H), 5.82 (d, J=5.4 Hz, 1H), 5.75 (s, OH), 5.45 (dd, J=8.1, 2.2 Hz, 1H), 5.21 (t, J=5.1 Hz, 1H), 4.26 (t, J=5.5 Hz, 1H), 4.17-4.09 (m, 3H), 3.74 (s, 6H), 3.50-3.33 (m, 3H), 3.26 (dd, J=10.8, 3.4 Hz, 1H), 2.64-2.52 (m, 2H), 2.52-2.48 (m, 4H), 1.69-1.61 (m, 2H), 1.47-1.35 (m, 4H), 1.30-1.22 (m, 7H) ppm. ¹³C NMR (151 MHz, DMSO) δ 173.16, 171.43, 163.29, 162.86, 158.18, 150.38, 144.52, 140.33, 135.20, 135.01, 134.72, 129.74, 129.72, 128.61, 127.95, 127.65, 126.85, 123.18, 113.31, 113.29, 102.11, 87.01, 86.10, 80.58, 78.37, 77.66, 70.44, 70.23, 62.72, 55.05, 54.91, 28.89, 28.67, 28.65, 28.58, 28.57, 27.65, 25.29, 25.08 ppm.

### CPG ELN0132-514:

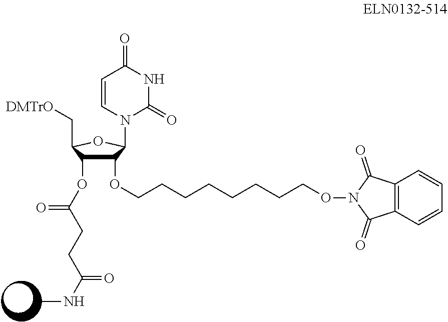

Added ELN0132-472 (0.4 g, 434.80 mol) and diisopropylethylamine (224.77 mg, 1.74 mmol, 302.93 μL) into rb flask. Then added dry acetonitrile (50 mL). Stirred well to dissolve and then HBTU (173.14 mg, 456.54 mol) to preactivate acid, stirred for ˜5 min, then added CPG. Capped and put on mechanical shaker overnight. Next day filtered CPG, washed with ACN, then MeOH, then ACN, then diethyl ether. Dried for ˜5 minutes, then transferred back to rb flask for capping. Added 30% acetic anhydride in pyridine (50 ml total) and 1% TEA. Capped and put back on mechanical shaker for 3 hours. After 3 hours took off and washed CPG as follows: 10% H₂O/THF, then MeOH, then 10% H₂O/THF, then MeOH, then ACN, the ether (˜250 mL each solvent for washing). Transferred to rb flask and dried CPG in high vacuum overnight.

Checking the Loading: Weighted out 39 mg and loaded into 250 ml volumetric flask. Then added 0.1M toluene-p-sulfonic acid in ACN up to measure line. Sonicated and settled for 1 hour. Checked loading by spectrophotometer and beers law. Measured solution into UV cuvette and measured UV absorbance at 411 nm. Check worksheet for raw data. Calculated loading using beers law=[250 (mL)×(absorbance A)×35.5 (extinction coefficient of DMTr)]/weight of CPG (mg). Yield: 4.15 g, Loading: 91 μmol/g.

### 3-[[(2R,5R)-5-[[bis(4-methoxyphenyl)-phenyl-methoxy]methyl]-4-[8-(1,3-dioxoisoindolin-2-yl)oxyoctoxy]-2-(2,4-dioxopyrimidin-1-yl)tetrahydrofuran-3-yl]oxy-(diisopropylamino)phos-phanyl]oxypropanenitrile ELN0132-406

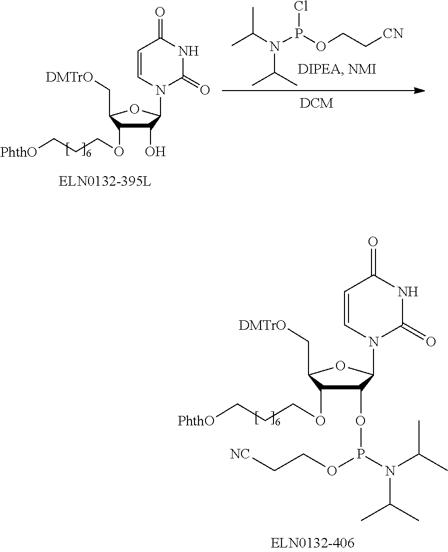

To a clear solution of ELN0132-395L (0.68 g, 829.38 mol) in DCM (20 mL) was added N-methylimidazole (102.14 mg, 1.24 mmol, 99.16 μL) and diisopropylethylamine (535.94 mg, 4.15 mmol, 722.30 μL) in single portions. After stirring the reaction mixture for 5 minutes at 22° C., 2-cyanoethyl-N,N-diisopropylchlorophosphoramidite (392.59 mg, 1.66 mmol, 370.37 μL) was added and continued stirring for 1 hr and TLC was checked. Starting material was consumed and reaction mixture was diluted with DCM (15 mL). DCM layer was washed with 10% NaHCO₃ (2×25 mL) solution, and brine (30 mL). Organic layer was separated, dried over anhydrous Na₂SO₄, filtered and filtrate was evaporated at 36° C. to afford crude compound which was purified by flash chromatography (30-70% EtOAc in hexane) to afford ELN0132-406 (0.71 g, 84% yield) as white foam. ¹H NMR (600 MHz, CD₃CN) δ 9.11-9.08 (m, 1H), 7.82-7.74 (m, 5H), 7.45-7.40 (m, 2H), 7.37-7.20 (m, 7H), 6.87 (dd, J=9.0, 3.1 Hz, 4H), 5.93-5.82 (m, 1H), 5.35-5.25 (m, 1H), 4.53-4.46 (m, 1H), 4.16-4.00 (m, 5H), 3.89-3.65 (m, 8H), 3.47-3.26 (m, 3H), 2.69-2.57 (m, 2H), 1.69 (dqt, J=9.4, 7.1, 3.5 Hz, 2H), 1.59-1.49 (m, 3H), 1.43 (p, J=7.5 Hz, 2H), 1.31 (dt, J=14.1, 4.3 Hz, 7H), 1.20-1.11 (m, 13H) ppm. ¹³C NMR (151 MHz, CD₃CN) δ 164.63, 163.90, 163.86, 159.72, 159.70, 159.68, 151.28, 151.22, 145.79, 145.74, 141.40, 141.13, 136.48, 136.46, 136.36, 136.29, 135.53, 131.06, 131.02, 131.00, 130.06, 128.94, 128.92, 127.98, 127.96, 123.97, 119.54, 119.53, 114.11, 114.09, 114.07, 102.48, 102.44, 89.76, 89.39, 89.36, 87.62, 87.51, 82.29, 82.22, 79.03, 77.57, 77.26, 77.22, 76.00, 75.90, 75.28, 75.17, 71.49, 71.47, 71.30, 63.01, 60.94, 59.81, 59.69, 59.37, 59.24, 55.89, 44.18, 44.10, 43.99, 43.90, 30.44, 30.02, 29.99, 29.87, 29.82, 28.84, 26.69, 26.63, 26.27, 26.24, 25.04, 24.99, 24.95, 24.90, 24.83, 24.81, 24.77, 21.13, 21.00, 20.96, 20.86, 20.81 ppm. ³¹P NMR (243 MHz, CD3CN) δ 150.74, 149.94 ppm. HRMS calc. for C₅₅H₆₇N₅O₁₂P [M+H]⁺ 1020.4524, found 1020.4528.

### 4-[(2R,5R)-5-[[bis(4-methoxyphenyl)-phenyl-methoxy]methyl]-4-[8-(1,3-dioxoisoindolin-2-yl)oxyoctoxy]-2-(2,4-dioxopyrimidin-1-yl)tetrahydrofuran-3-yl]oxy-4-oxo-butanoic acid ELN0132-473

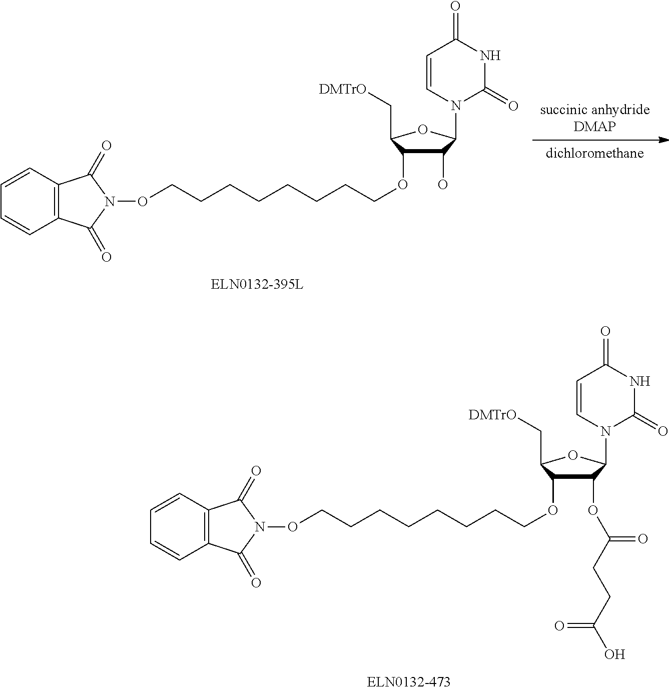

To a clear solution of ELN0132-395L (0.2 g, 243.93 mol) in dichloromethane (10 mL) was added DMAP (119.21 mg, 975.74 mol) and succinic anhydride (73.23 mg, 731.80 mol) sequentially. The resulting mixture was stirred for 3 hr at 22° C. Rection mixture was diluted with DCM (15 mL) and organic layer was washed with 10% NH₄Cl solution (2×20 mL). Organic layer was separated, dried over anhydrous Na₂SO₄, filtered and the filtrate was evaporated to dryness. The crude mass thus obtained was purified by flash column chromatography (gradient: 0-5% Methanol in DCM) for afford ELN0132-473 (0.16 g, 67% yield) as white foam. ¹H NMR (600 MHz, DMSO) δ 12.22 (s, 1H), 11.40 (d, J=2.2 Hz, 1H), 7.85 (s, 4H), 7.77 (d, J=8.1 Hz, 1H), 7.40-7.35 (m, 2H), 7.31 (dd, J=8.5, 7.0 Hz, 2H), 7.27-7.21 (m, 5H), 6.92-6.86 (m, 4H), 5.82 (d, J=3.0 Hz, 1H), 5.46 (dd, J=5.3, 3.0 Hz, 1H), 5.38 (dd, J=8.1, 2.2 Hz, 1H), 4.24 (dd, J=7.3, 5.3 Hz, 1H), 4.11 (t, J=6.5 Hz, 2H), 3.97 (dt, J=7.2, 3.5 Hz, 1H), 3.73 (s, 6H), 3.42 (dt, J=9.4, 6.5 Hz, 1H), 3.35-3.33 (m, 1H), 3.23 (dd, J=10.9, 4.0 Hz, 1H), 2.66-2.53 (m, 2H), 2.50-2.43 (m, 4H), 1.68-1.60 (m, 2H), 1.44-1.33 (m, 4H), 1.28-1.15 (m, 7H) ppm. ¹³C NMR (151 MHz, DMSO) δ 173.36, 173.10, 172.59, 171.11, 163.29, 163.08, 158.16, 150.15, 144.55, 140.93, 135.21, 135.12, 134.73, 129.75, 129.73, 128.60, 127.89, 127.68, 126.81, 123.18, 113.23, 101.65, 87.95, 85.92, 80.50, 77.65, 75.38, 73.35, 70.49, 61.89, 55.03, 51.37, 29.09, 28.71, 28.64, 28.59, 28.50, 27.66, 25.37, 25.09 ppm.

### CPG ELN0132-515:

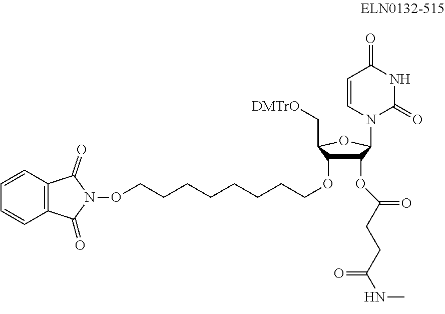

Added ELN0132-473 (0.15 g, 163.05 mol) and diisopropylethylamine (84.29 mg, 652.20 mol, 113.60 μL) into rb flask. Then added dry acetonitrile (30 mL). Stirred well to dissolve and then HBTU (64.93 mg, 171.20 mol) to preactivate acid. Let stir for ˜5 min, then added CPG. Capped and put on mechanical shaker overnight. Next day filtered CPG, washed with ACN, then MeOH, then ACN, then diethyl ether. Dried for ˜5 minutes, then transferred back to rb flask for capping. Added 30% acetic anhydride in pyridine (50 ml total) and 1% TEA. Capped and put back on mechanical shaker for 3 hours. After 3 hours took off and washed CPG as follows: 10% H₂O/THF, then MeOH, then 10% H₂O/THF, then MeOH, then ACN, the ether (˜250 mL each solvent for washing). Transferred to rb flask and dried CPG in high vacuum overnight.

Checking the Loading: Weighted out 42 mg and loaded into 250 ml volumetric flask. Then added 0.1M toluene-p-sulfonic acid in ACN up to measure line. Sonicated and settled for 1 hour. Checked loading by spectrophotometer and beers law. Measured solution into UV cuvette and measured UV absorbance at 411 nm. Check worksheet for raw data. Calculated loading using beers law=[250 (mL) x (absorbance A)×35.5 (extinction coefficient of DMTr)]/weight of CPG (mg). Yield: 1.59 g, Loading: 93 μmol/g.

### Compounds for Incorporation in Oligo-Synthesis Including AOCC

2′-OMe-U analouges Under the Mitsunobu reaction condition with N-hydroxylphthalimide (PhthOH), triphenylphosphine (TPP) and diethyl azodicarboxylate (DEAD), compound 1 was converted to 9 in good yield. Compound 9 was then treated with N-methylhydrazine to afford 10 with aminooxy (—ONH₂) group at the 5′-end. Reaction of compound 10 with the carbonyl groups of benzyl-4-oxopiperidine-1-carboxylate and tetrahydropyran-4-one under acidic conditions produced compound 11 and 12 respectively in good yields. Removal of TBS groups from 11 and 12 through TBAF afforded corresponding alcohols 13 and 14. Finally, phosphitylation reaction of 13 and 14 with 2-cyanoethyl-N,N-diisopropylchlorophosphoramidite resulted in desired amidites 15 and 16 respectively in moderate yields. (Scheme 17)

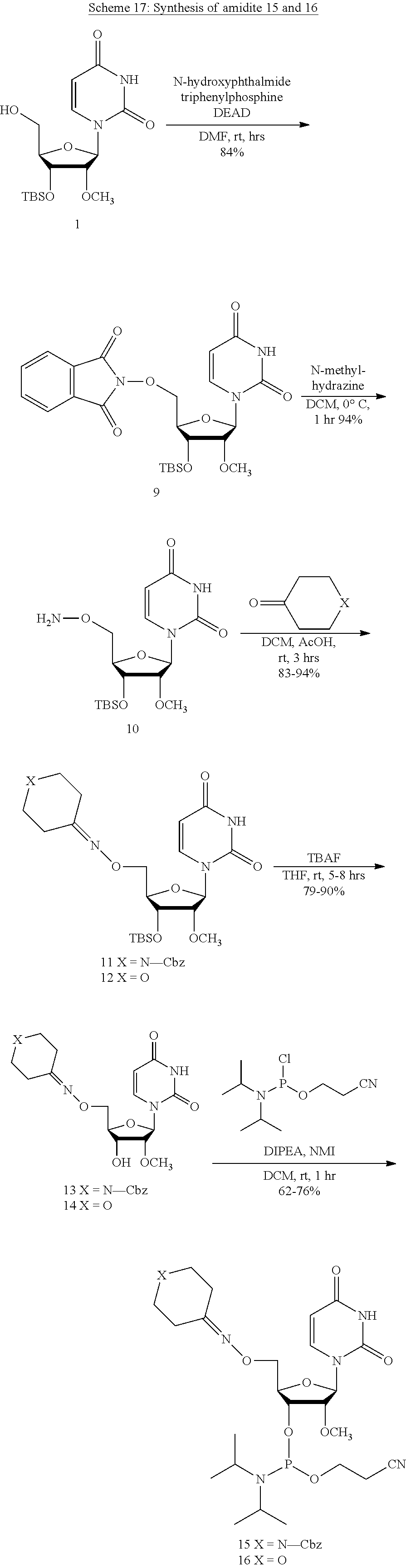

Reductive amination of 10 with glutaraldehyde resulted in compound 17 with a six-membered heterocyclic ring comprising of the aminooxy nitrogen atom. Removal of TBS protecting group in presence of TBAF from 17 afforded compound 18. Phosphitylation reaction of 18 produced amidite 19 in moderate yield (Scheme 18).

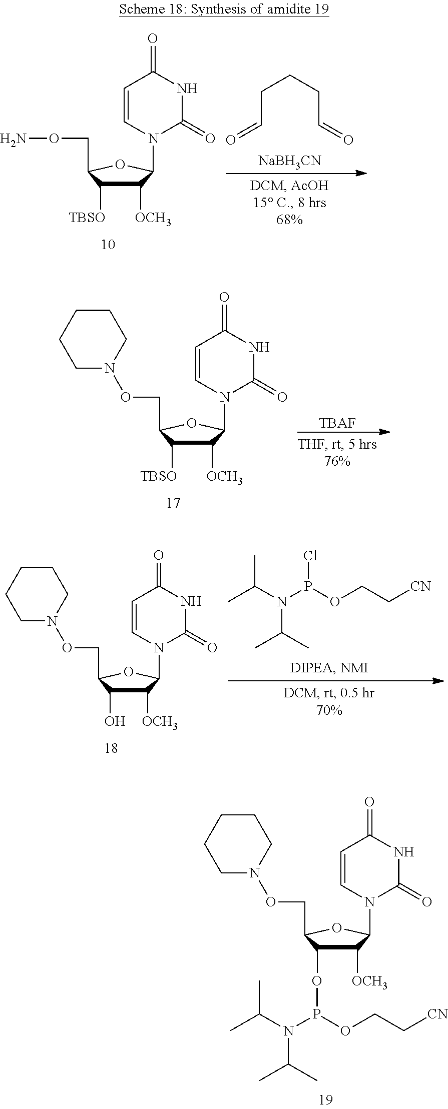

Reaction of 2-(2-oxoethoxy)acetaldehyde³² with compound 10 under similar reductive amination condition, produced 20, the cyclic six-membered heterocyclic structure containing the aminooxy nitrogen atom at 5′-position, however, in low yield. Compound 20, upon deprotection of silyl group with TBAF resulted in compound 21 in good yield. Finally, phosphitylation of 21 with 2-cyanoethyl-N,N,N′,N′-tetraisopropylphosphordiamidite and 5-(ethylthio)-1H-tetrazole (ETT) afforded the amidite 22 in moderate yield. (Scheme 19)

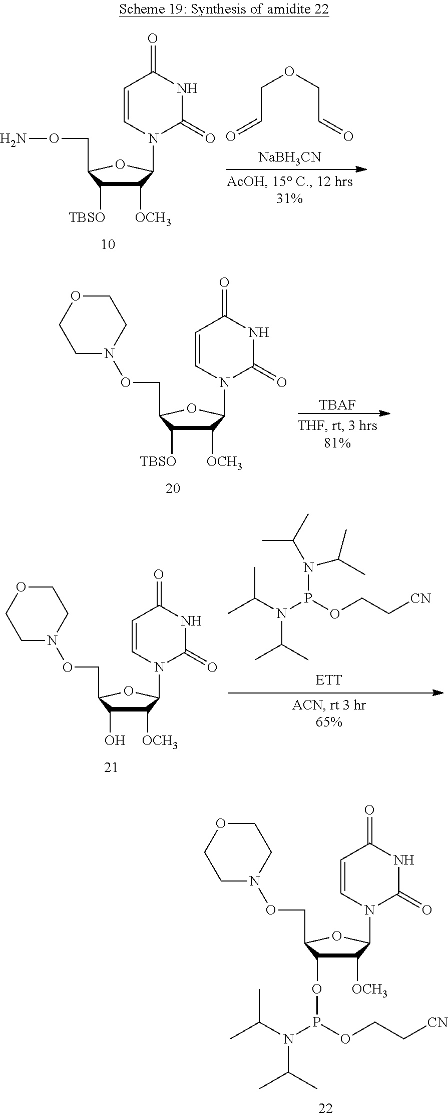

### 2-[[(2R,5R)-3-[tert-butyl(dimethyl)silyl]oxy-5-(2,4-dioxopyrimidin-1-yl)-4-methoxy-tetrahydrofuran-2-yl]methoxy]isoindoline-1,3-dione (9)

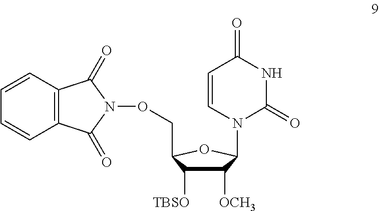

To a solution of 1 (5 g, 13.42 mmol) in dimethylformamide (60 mL) was added triphenylphosphine (4.82 g, 17.45 mmol) and N-hydroxylphthalimide (2.93 g, 17.45 mmol). To this resulting mixture, diethyl azodicarboxylate (3.20 g, 17.45 mmol, 3.35 mL) was added dropwise at 0° C. The reaction mixture was stirred at 22° C. for 4 hrs. The reaction mixture was then quenched with 10% aqueous NaHCO₃ (50 mL) and extracted with EA (3×50 mL). Combined organic layer was dried over anhydrous Na₂SO₄, filtered and the filtrated was evaporated to dryness. The crude residue thus obtained was purified by column chromatography (gradient: 0-40% EA in hexane) to afford 9 (5.8 g, 84% yield) as white solid. ¹H NMR (400 MHz, CDCl₃) δ 9.92 (s, 1H), 8.09 (d, J=8.2 Hz, 1H), 7.89-7.81 (m, 2H), 7.80-7.68 (m, 2H), 5.91 (d, J=2.9 Hz, 1H), 5.77 (dd, J=8.1, 2.1 Hz, 1H), 4.56 (dd, J=10.4, 2.7 Hz, 1H), 4.49-4.37 (m, 2H), 4.23 (dt, J=6.9, 2.4 Hz, 1H), 3.76 (dd, J=4.9, 2.9 Hz, 1H), 3.53 (s, 3H), 0.90 (s, 9H), 0.14 (d, J=8.3 Hz, 6H) ppm. ¹³C NMR (101 MHz, CDCl₃) δ 163.92, 163.87, 163.14, 150.56, 140.54, 134.84, 128.84, 123.79, 102.49, 88.39, 83.51, 81.91, 75.93, 69.20, 69.18, 58.53, 25.76, 18.17, −4.67, −4.89 ppm. HRMS calc. for C₂₄H₃₁N₃O₈SiNa [M+Na]+, 540.1778, found 540.1768.

### 1-[(2R,5R)-5-(aminooxymethyl)-4-[tert-butyl(dimethyl)silyl]oxy-3-methoxy-tetrahydrofuran-2-yl]pyrimidine-2,4-dione (10)

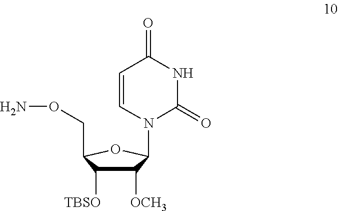

A solution of 9 (2.0 g, 3.86 mmol) in DCM (25 mL) was treated with methylhydrazine (0.21 g, 4.64 mmol, 0.24 mL) at 0° C. for 1 hr with stirring. The reaction mixture was filtered, washed with cold DCM and combined organic solvent was washed with water (3×20 mL). Organic layer was separated, dried over anhydrous Na₂SO₄, filtered and the filtrated was evaporated to dryness to obtain white solid. Crude material thus obtained was purified by column chromatography (gradient: 0-5% MeOH in DCM) to afford 10 (1.4 g, 94% yield). ¹H NMR (400 MHz, DMSO-d₆) δ 11.39 (d, J=2.2 Hz, 1H), 7.72 (d, J=8.1 Hz, 1H), 6.21 (s, 2H), 5.80 (d, J=4.9 Hz, 1H), 5.67 (dd, J=8.1, 2.1 Hz, 1H), 4.26 (t, J=4.9 Hz, 1H), 3.96 (td, J=4.8, 3.3 Hz, 1H), 3.84 (t, J=5.0 Hz, 1H), 3.79-3.65 (m, 2H), 3.33 (s, 3H), 0.88 (s, 9H), 0.09 (d, J=1.5 Hz, 6H). ¹³C NMR (126 MHz, DMSO-d₆) δ 162.99, 150.44, 140.51, 102.13, 86.65, 82.42, 81.64, 74.57, 70.23, 57.56, 25.62, 17.78, −4.82, −4.98 ppm. HRMS calc. for C₁₆H₃₀N₃O₆Si [M+H]⁺, 388.1904, found 388.1901.

### Benzyl-4-[[(2R,5R)-3-[tert-butyl(dimethyl)silyl]oxy-5-(2,4-dioxopyrimidin-1-yl)-4-methoxy-tetrahydrofuran-2-yl]methoxyimino]piperidine-1-carboxylate (11)

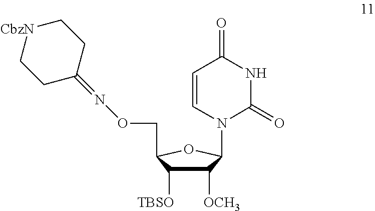

To a solution of 10 (0.55 g, 1.42 mmol) in DCM (10 mL), benzyl-4-oxopiperidine-1-carboxylate (0.67 g, 2.84 mmol, 0.57 mL) was added. To the resulting mixture, glacial acetic acid (5 mL) was added in single portion and stirred for 3 hrs at 22° C. Reaction mixture was diluted with DCM (20 mL) and water (30 mL) was added. Organic layer was separated, dried over anhydrous Na₂SO₄, filtered and filtrate was evaporated to dryness. Crude compound was purified by column chromatography (gradient: 0-50% EA in hexane) to afford 11 (0.8 g, 94% yield) as white foam. ¹H NMR (400 MHz, CDCl₃) δ 9.53 (s, 1H), 7.69 (d, J=8.1 Hz, 1H), 7.42-7.29 (m, 5H), 5.82 (d, J=1.9 Hz, 1H), 5.65 (d, J=8.1 Hz, 1H), 5.16 (s, 2H), 4.53-4.35 (m, 1H), 4.25-4.09 (m, 3H), 3.74-3.40 (m, 8H), 2.54 (s, 2H), 2.41-2.25 (m, 2H), 0.89 (s, 9H), 0.07 (d, J=5.3 Hz, 6H).ppm. ¹³C NMR (101 MHz, CDCl₃) δ 163.49, 156.49, 155.22, 150.16, 139.84, 136.55, 128.68, 128.30, 128.20, 101.97, 88.80, 83.77, 82.46, 71.52, 69.57, 67.64, 58.62, 44.04, 42.23, 30.93, 25.80, 18.26, −4.58, −4.80 ppm. HRMS calc. for C₂₉H₄₃N₄O₈Si [M+H]⁺ 603.2850, found 603.2857.

### 1-[(2R,5R)-4-[tert-butyl(dimethyl)silyl]oxy-3-methoxy-5-[(tetrahydropyran-4-ylideneamino) oxymethyl]tetrahydrofuran-2-yl]pyrimidine-2,4-dione (12)

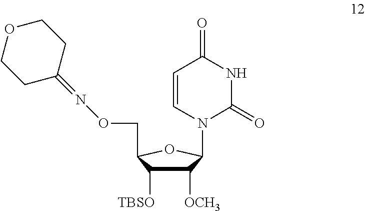

To a solution of 10 (0.25 g, 0.65 mmol) in DCM (7 mL), tetrahydropyran-4-one (0.130 g, 1.29 mmol, 0.121 mL) was added. To the resulting mixture, glacial acetic acid (3 mL) was added in single portion and stirred for 3 hrs at 22° C. Reaction mixture was diluted with DCM (20 mL) and water (30 mL) was added. Organic layer was separated, dried over anhydrous Na₂SO₄, filtered and filtrate was evaporated to dryness. Crude compound was purified by column chromatography (gradient: 20-80% EA in hexane) to afford 12 (0.25 g, 83% yield) as white foam. ¹H NMR (400 MHz, CDCl₃) δ 9.17 (s, 1H), 7.75 (d, J=8.1 Hz, 1H), 5.85 (d, J=2.0 Hz, 1H), 5.64 (dd, J=8.1, 2.2 Hz, 1H), 4.55-4.33 (m, 1H), 4.35-4.08 (m, 3H), 3.89-3.70 (m, 5H), 3.65 (q, J=2.0 Hz, 1H), 3.56 (s, 3H), 2.73-2.49 (m, 2H), 2.45-2.19 (m, 2H), 0.91 (s, 9H), 0.09 (d, J=4.2 Hz, 6H) ppm. ¹³C NMR (101 MHz, CDCl₃) δ 163.35, 156.03, 150.08, 139.97, 101.91, 88.69, 83.90, 82.64, 71.35, 69.55, 68.37, 66.65, 58.66, 32.35, 27.15, 25.83, 18.30, −4.56, −4.76 ppm. HRMS calc. for C₂₁H₃₆N₃₀₇Si [M+H]⁺ 470.2323, found 470.2317.

### Benzyl-4-[[(2R,5R)-5-(2,4-dioxopyrimidin-1-yl)-3-hydroxyl-4-methoxy-tetrahydrofuran-2-yl]methoxyimino]piperidine-1-carboxylate (13)

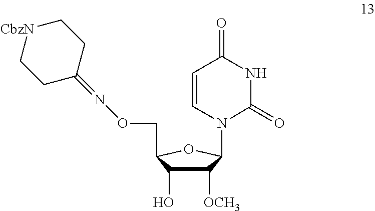

To a solution of 11 (0.8 g, 1.33 mmol) in THF (10 mL) at 22° C., tetrabutylammonium fluoride, 1M in THF (1.99 mmol, 1.99 μL) was added slowly in single portion and then stirred for 5 hrs. Volatile matters were removed in high vacuum pump and crude residue thus obtained was purified by column chromatography (gradient: 0-5% MeOH in DCM) to afford 13 (0.58 g, 90% yield) as white solid. ¹H NMR (500 MHz, CDCl₃) δ 9.24 (s, 1H), 7.65 (d, J=8.2 Hz, 1H), 7.37 (d, J=4.4 Hz, 4H), 7.33 (dt, J=5.8, 4.1 Hz, 1H), 5.89 (d, J=1.8 Hz, 1H), 5.67 (d, J=8.1 Hz, 1H), 5.16 (s, 2H), 4.48 (dd, J=12.4, 2.4 Hz, 1H), 4.31 (dd, J=12.4, 3.5 Hz, 1H), 4.24-4.03 (m, 2H), 3.76 (dd, J=5.2, 1.9 Hz, 1H), 3.69-3.50 (m, 7H), 2.79 (d, J=8.5 Hz, 1H), 2.56 (s, 2H), 2.37 (t, J=6.1 Hz, 2H) ppm. ¹³C NMR (126 MHz, CDCl₃) δ 163.23, 156.85, 155.25, 150.12, 139.53, 136.56, 128.69, 128.32, 128.20, 102.22, 87.99, 83.68, 82.95, 77.41, 77.36, 77.16, 76.91, 76.66, 72.00, 68.82, 67.64, 58.91, 44.00, 42.18, 30.92, 29.83, 25.80, 0.13 ppm. HRMS calc. for C₂₃H₂₉N₄O₈ [M+H]⁺ 489.1985, found 489.1995.

### 1-[(2R,5R)-4-hydroxyl-3-methoxy-5-[(tetrahydropyran-4-ylideneamino)oxymethyl]tetrahydrofuran-2-yl]pyrimidine-2,4-dione (14)

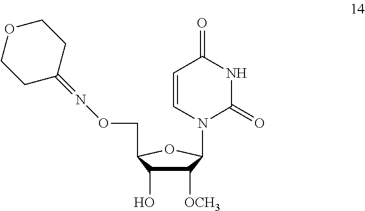

To a clear solution of 12 (0.21 g, 0.45 mmol) in THF (10 mL) at 22° C., Tetrabutylammonium fluoride, 1M in THF (0.68 mmol, 0.68 mL) was added slowly in single portion and then stirred for 8 hrs. Volatile matters were removed in high vacuum pump and crude residue thus obtained was purified by column chromatography (gradient: 30-80% EA in hexane) to afford 14 (0.126 g, 79% yield). ¹H NMR (500 MHz, CDCl₃) δ 8.99 (s, 1H), 7.70 (d, J=8.2 Hz, 1H), 5.91 (d, J=1.8 Hz, 1H), 5.66 (dd, J=8.1, 1.8 Hz, 1H), 4.48 (dd, J=12.5, 2.3 Hz, 1H), 4.32 (dd, J=12.5, 3.3 Hz, 1H), 4.20 (td, J=8.1, 5.2 Hz, 1H), 4.14 (dt, J=7.8, 3.0 Hz, 1H), 3.82 (q, J=5.3 Hz, 2H), 3.78-3.71 (m, 3H), 3.63 (s, 3H), 2.75 (d, J=8.5 Hz, 1H), 2.60 (q, J=5.6 Hz, 2H), 2.38 (t, J=5.6 Hz, 2H) ppm. ¹³C NMR (126 MHz, CDCl₃) δ 163.13, 156.39, 150.07, 139.59, 102.16, 87.90, 83.77, 83.09, 71.79, 68.78, 68.33, 66.64, 58.92, 32.34, 27.10 ppm. HRMS calc. for C₁₅H₂₂N₃O₇ [M+H]⁺ 356.1458, found 356.1464.

### Benzyl-4-[[(2R,5R)-3-[2-cyanoethoxy-(diisopropylamino)phosphanyl]oxy-5-(2,4-dioxopyrimidin-1-yl)-4-methoxy-tetrahydrofuran-2-yl]methoxyimino]piperidine-1-carboxylate (15)

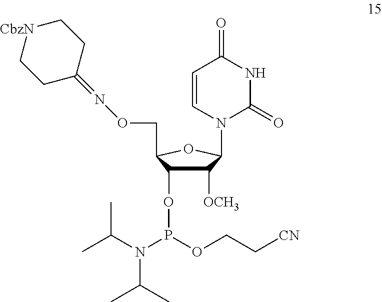

To a clear solution of 13 (0.49 g, 1.00 mmol) in DCM (20 mL), diisopropylethylamine (0.655 g, 5.02 mmol, 0.882 mL) and N-methylimidazole (0.29 g, 3.51 mmol, 0.283 mL) were added at 22° C. To this reaction mixture, 2-cyanoethyl-N,N-diisopropylchlorophosphoramidite (0.50 g, 2.01 mmol, 0.47 mL) was added slowly after 5 minutes and stirred for 1 hr. Reaction mixture was diluted with DCM (10 mL) and quenched with 10% NaHCO₃ solution (20 mL). Organic layer was separated, dried on anhydrous Na₂SO₄, filtered and filtrate was evaporated to dryness. The crude compound was thus obtained was purified by column chromatography (gradient: 10-60% EA in hexane) to afford 15 (0.43 g, 62% yield) as white solid. ¹H NMR (400 MHz, CDCl₃) δ 8.36 (brs, 1H), 7.63 (dd, J=8.2, 2.1 Hz, 1H), 7.42-7.30 (m, 5H), 5.92 (dd, J=10.1, 3.4 Hz, 1H), 5.64 (dd, J=8.1, 5.2 Hz, 1H), 5.16 (s, 2H), 4.55-4.33 (m, 2H), 4.35-4.13 (m, 2H), 3.94-3.77 (m, 2H), 3.78-3.57 (m, 7H), 3.52 (d, J=20.2 Hz, 3H), 2.95-2.36 (m, 4H), 2.35 (s, 2H), 1.18 (ddd, J=12.7, 7.8, 6.3 Hz, 12H) ppm. ¹³C NMR (126 MHz, CDCl₃) δ 162.82, 156.71, 155.25, 150.00, 139.80, 139.75, 136.58, 136.56, 128.72, 128.36, 128.35, 128.24, 117.80, 117.67, 102.33, 102.23, 88.55, 88.12, 83.20, 83.06, 83.03, 82.47, 82.44, 82.10, 82.06, 72.39, 71.99, 70.87, 70.75, 69.85, 69.71, 67.67, 67.65, 58.88, 58.82, 58.80, 58.74, 58.54, 58.52, 58.12, 57.96, 44.03, 43.61, 43.52, 43.49, 43.39, 42.20, 30.93, 25.87, 24.81, 24.79, 24.76, 24.74, 24.72, 24.68, 20.60, 20.56, 20.54, 20.51 ppm. ³¹P NMR (162 MHz, CDCl₃) δ 150.85, 150.51 ppm. HRMS calc. for C₃₂H₄₅N₆₀₉PNa [M+Na]⁺ 711.2883, found 711.2886.

### 1-[(2R,5R)-4-[but-3-ynoxy-(diisopropylamino)phosphanyl]oxy-3-methoxy-5-[(tetrahydropyran -4-ylideneamino)oxymethyl]tetrahydrofuran-2-yl]pyrimidine-2,4-dione (16)

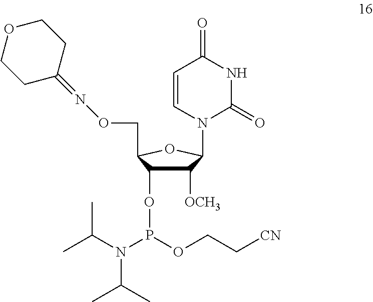

To a clear solution of 14 (0.11 g, 0.309 mmol) in DCM (5 mL), diisopropylethylamine (0.20 g, 1.55 mmol, 0.27 mL) and N-methylimidazole (0.089 g, 1.08 mmol, 0.086 mL) were added at 22° C. To this reaction mixture, 2-cyanoethyl-N,N-diisopropylchlorophosphoramidite (0.154 g, 0.62 mmol, 0.146 mL) was added slowly after 5 minutes and stirred for 1 hr. Reaction mixture was diluted with DCM (10 mL) and quenched with 10% NaHCO₃ solution (20 mL). Organic layer was separated, dried on anhydrous Na₂SO₄, filtered and filtrate was evaporated to dryness. The crude compound was thus obtained was purified by column chromatography (gradient: 10-60% EA in hexane) to afford 16 (0.13 g, 76% yield). ¹H NMR (500 MHz, CDCl₃) δ 8.59 (s, 1H), 7.67 (dd, J=8.2, 2.3 Hz, 1H), 5.94 (dd, J=10.0, 3.3 Hz, 1H), 5.64 (dd, J=8.1, 6.0 Hz, 1H), 4.52-4.35 (m, 2H), 4.36-4.20 (m, 2H), 3.98-3.59 (m, 9H), 3.53 (d, J=24.7 Hz, 3H), 2.69-2.48 (m, 4H), 2.47-2.21 (m, 2H), 1.35-1.01 (m, 14H) ppm. ¹³C NMR (126 MHz, CD₃CN) δ 163.84, 163.80, 157.53, 151.37, 151.33, 140.59, 119.59, 119.58, 103.00, 102.87, 88.47, 87.99, 83.67, 83.65, 83.60, 83.57, 83.35, 83.31, 83.13, 83.09, 73.15, 72.84, 71.84, 71.72, 71.08, 70.94, 68.77, 67.16, 67.14, 59.90, 59.75, 59.30, 59.14, 58.97, 58.95, 58.70, 58.68, 44.23, 44.13, 44.03, 32.83, 27.79, 27.77, 27.25, 24.98, 24.97, 24.95, 24.91, 24.89, 24.85, 21.07, 21.01, 20.96 ppm. ³¹P NMR (202 MHz, CD₃CN) δ 151.50, 151.28 ppm. HRMS calc. for C₂₄H₃₈N₅O₈PNa [M+Na]⁺ 578.2356, found 578.2359.

### 1-[(2R,5R)-4-[tert-butyl(dimethyl)silyl]oxy-3-methoxy-5-(1-piperidyloxymethyl)tetrahydrofuran-2-yl]pyrimidine-2,4-dione (17)

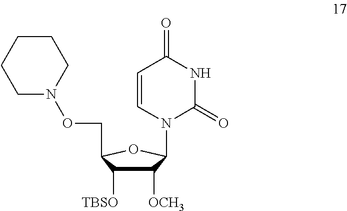

To a clear solution of 10 (0.4 g, 1.03 mmol) in acetic acid (5 mL) and DCM (10 mL), was added glutaraldehyde (0.1 g, 1.03 mmol). To the resulting mixture, sodium cyanoborohydride (0.74 g, 11.56 mmol) was added in portions at 15° C. The reaction mixture was further diluted with DCM (70 mL) and stirred for 8 hrs. Volatile matters were removed under high vacuum and the residue thus obtained, was diluted with DCM (50 mL) and washed with water (3×30 mL). Organic layer was separated, dried over anhydrous Na₂SO₄, filtered and filtrate was evaporated to dryness. The crude compound thus obtained was purified by column chromatography to afford 17 (0.32 g, 68.0% yield). ¹H NMR (400 MHz, CDCl₃) δ 9.18 (s, 1H), 8.07 (d, J=8.2 Hz, 1H), 5.88 (d, J=1.9 Hz, 1H), 5.69 (dd, J=8.1, 2.1 Hz, 1H), 4.36-3.96 (m, 3H), 3.97-3.72 (m, 1H), 3.61 (dd, J=4.7, 1.9 Hz, 1H), 3.54 (s, 3H), 3.41-3.24 (m, 2H), 2.36 (s, 2H), 1.74 (s, 2H), 1.56 (d, J=18.3 Hz, 3H), 1.24-1.11 (m, 1H), 0.90 (s, 9H), 0.09 (d, J=3.4 Hz, 6H) ppm. ¹³C NMR (126 MHz, CDCl₃) δ 163.51, 150.24, 140.47, 101.66, 88.13, 84.22, 82.51, 69.37, 68.69, 58.51, 56.91, 25.83, 25.49, 23.50, 18.26, −4.48, −4.73 ppm. HRMS calc. for C₂₁H₃₈N₃O₆Si [M+H]⁺ 456.2530, found 456.2520.

### 1-[(2R,5R)-4-hydroxyl-3-methoxy-5-(1-piperidyloxymethyl)tetrahydrofuran-2-yl]pyrimidine-2,4-dione (18)

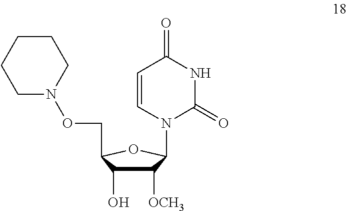

To a solution of 17 (0.30 g, 0.66 mmol) in THF (5 mL) at 25° C., Tetrabutylammonium fluoride, 1M in THF (0.99 mmol, 0.99 mL) was added slowly in single portion and then stirred for 5 hrs. Volatile matters were removed in high vacuum pump and crude residue thus obtained was purified by column chromatography (gradient: 10-60% EA in hexane) to afford 18 (0.17 g, 76% yield). ¹H NMR (400 MHz, CDCl₃) δ 8.79 (s, 1H), 7.97 (d, J=8.2 Hz, 1H), 5.94 (d, J=2.2 Hz, 1H), 5.71 (dd, J=8.2, 1.8 Hz, 1H), 4.20 (td, J=7.4, 5.2 Hz, 1H), 4.16-4.11 (m, 1H), 4.08 (dt, J=7.1, 2.6 Hz, 1H), 3.95 (dd, J=11.2, 2.7 Hz, 1H), 3.77 (dd, J=5.2, 2.3 Hz, 1H), 3.61 (s, 3H), 3.36 (s, 2H), 2.84 (d, J=7.7 Hz, 1H), 2.39 (t, J=11.4 Hz, 2H), 1.76 (d, J=13.0 Hz, 2H), 1.59 (brs, 2H) ppm. ¹³C NMR (101 MHz, CDCl₃) δ 163.24, 150.21, 140.20, 102.02, 87.59, 83.97, 83.15, 69.46, 69.08, 58.83, 57.03, 56.72, 25.47, 23.49 ppm. HRMS calc. for C₁₅H₂₄N₃O₆ [M+H]⁺ 342.1665, found 342.1656.

### 3-[(diisopropylamino)-[(2R,5R)-5-(2,4-dioxopyrimidin-1-yl)-4-methoxy-2-(1-piperidyloxy methyl)tetrahydrofuran-3-yl]oxy-phosphanyl]oxypropanenitrile (19)

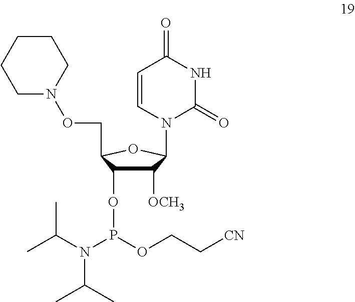

To a clear solution of 18 (0.60 g, 1.76 mmol) in DCM (20 mL), diisopropylethylamine (1.15 g, 8.79 mmol, 1.55 mL) and N-methylimidazole (0.51 g, 6.15 mmol, 0.49 mL) were added at 22° C. To this reaction mixture, 2-cyanoethyl-N,N-diisopropylchlorophosphoramidite (0.88 g, 3.52 mmol, 0.82 mL) was added slowly after 5 minutes and stirred for 0.5 hr. Reaction mixture was diluted with DCM (10 mL) and quenched with 10% NaHCO₃ solution (20 mL). Organic layer was separated, dried on anhydrous Na₂SO₄, filtered and filtrate was evaporated to dryness. The crude compound was thus obtained was purified by column chromatography (gradient: 20-80% EA in hexane) to afford 19 (0.66 g, 70% yield) as hygroscopic solid. ¹H NMR (400 MHz, CDCl₃) δ 8.81 (s, 1H), 7.96 (dd, J=10.9, 8.1 Hz, 1H), 5.97 (d, J=3.7 Hz, 1H), 5.69 (d, J=8.1 Hz, 1H), 4.49-4.16 (m, 2H), 4.09 (td, J=11.2, 2.3 Hz, 1H), 3.98-3.77 (m, 4H), 3.72-3.58 (m, 2H), 3.51 (d, J=14.2 Hz, 3H), 3.39-3.23 (m, 2H), 2.64 (dt, J=11.9, 6.3 Hz, 2H), 2.39 (d, J=10.5 Hz, 2H), 1.73 (s, 2H), 1.58 (s, 3H), 1.30-1.06 (m, 15H) ppm. ¹³C NMR (101 MHz, CDCl₃) δ 163.39, 163.37, 150.36, 150.34, 140.37, 140.25, 117.81, 117.64, 102.08, 102.04, 87.80, 87.52, 83.48, 83.45, 83.15, 83.11, 82.45, 82.42, 82.22, 82.16, 70.90, 70.74, 70.15, 69.98, 69.56, 69.52, 58.92, 58.74, 58.71, 58.35, 58.32, 58.18, 58.07, 58.00, 56.89, 53.56, 43.56, 43.50, 43.44, 43.37, 25.45, 24.79, 24.75, 24.72, 24.71, 24.68, 23.47, 23.45, 20.57, 20.53, 20.49, 20.46 ppm. ³¹P NMR (162 MHz, CDCl₃) δ 150.98, 150.54 ppm. HRMS calc. for C₂₄H₄₁N₅O₇P [M+H]⁺ 542.2744, found 542.2747.

### 1-[(2R,5R)-4-[tert-butyl(dimethyl)silyl]oxy-3-methoxy-5-(morpholinooxymethyl)tetrahydrofuran-2-yl]pyrimidine-2,4-dione (20)

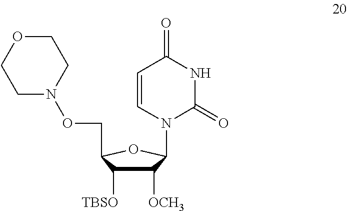

To a clear solution of 10 (2.4 g, 6.19 mmol) in acetic acid (20 mL) was added 2-(2-oxoethoxy)acetaldehyde³² (0.63 g, 6.19 mmol) in followed by sodium cyanoborohydride (4.13 g, 64.4 mmol) in portions and stirred at 15° C. for 12 hrs. Diluted the mixture with DCM (50 mL) and organic layer was washed with water (2×30 mL). DCM layer dried over anhydrous Na₂SO₄, filtered and the filtrate was evaporated to dryness. The crude residue was purified by column chromatography to afford 20 (0.88 g, 31.0% yield) as white solid. ¹H NMR (500 MHz, Chloroform-d) δ 8.50 (s, 1H), 7.93 (d, J=8.2 Hz, 1H), 5.86 (d, J=2.0 Hz, 1H), 5.70 (dd, J=8.2, 2.0 Hz, 1H), 4.23-4.06 (m, 3H), 3.92 (dd, J=11.9, 3.0 Hz, 3H), 3.67-3.57 (m, 3H), 3.55 (s, 3H), 3.23 (dd, J=28.2, 10.2 Hz, 2H), 2.65 (q, J=10.1 Hz, 2H), 0.91 (s, 9H), 0.10 (d, J=5.8 Hz, 6H) ppm. 13C NMR (126 MHz, CDCl₃) δ 163.36, 150.14, 140.21, 101.81, 88.42, 84.06, 82.14, 69.45, 68.95, 66.36, 58.56, 56.41, 25.82, 18.26, −4.44, −4.72 ppm. HRMS calc. for C₂₀H₃₆N₃O₇Si [M+H]⁺ 458.2323, found 458.2315.

### 1-[(2R,5R)-4-hydroxyl-3-methoxy-5-(morpholinooxymethyl)tetrahydrofuran-2-yl]pyrimidine-2,4-dione (21)

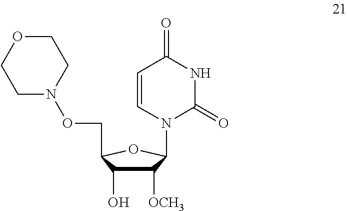

To a solution of 20 (0.85 g, 1.86 mmol) in THF (15 mL) at 22° C., tetrabutylammonium fluoride, 1M in THF (2.41 mmol, 2.41 mL) was added slowly in single portion and then stirred for 3 hrs. Volatile matters were removed in high vacuum pump and crude residue thus obtained was purified by column chromatography (gradient: 0-5% MeOH in DCM) to afford 21(0.516 g, 80.9% yield) as white solid. ¹H NMR (500 MHz, CDCl₃) δ 9.26 (s, 1H), 7.85 (d, J=8.2 Hz, 1H), 5.93 (d, J=2.1 Hz, 1H), 5.73 (d, J=8.2 Hz, 1H), 4.23-4.14 (m, 2H), 4.08 (dt, J=7.3, 2.9 Hz, 1H), 3.98 (dd, J=11.3, 3.2 Hz, 1H), 3.92 (d, J=11.7 Hz, 2H), 3.81-3.71 (m, 1H), 3.62 (s, 5H), 3.25 (t, J=9.1 Hz, 2H), 2.81 (d, J=8.3 Hz, 1H), 2.67 (td, J=10.9, 3.2 Hz, 2H) ppm. ¹³C NMR (101 MHz, CDCl₃) δ 163.38, 150.24, 139.96, 102.15, 87.72, 83.74, 82.75, 69.61, 68.86, 66.32, 58.86, 56.50, 56.23 ppm. HRMS calc. for C₁₄H₂₂N₃O₇ [M+H]⁺ 344.1458, found 344.1465.

### 3-[(diisopropylamino)-[(2R,5R)-5-(2,4-dioxopyrimidin-1-yl)-4-methoxy-2-(morpholinooxy methyl)tetrahydrofuran-3-yl]oxy-phosphanyl]oxypropanenitrile (22)

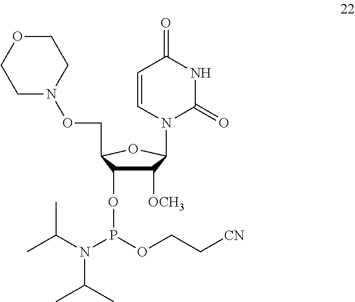

Phosphitylation of 21 following the literature procedure³³ afforded 22. To a solution of 21 (0.3 g, 0.87 mmol) in dry acetonitrile (10 mL) was added 5-(ethylthio)-1H-tetrazole (0.12 g, 0.87 mmol). 2-Cyanoethyl-N,N,N′,N′-tetraisopropylphosphordiamidite (1.14 mmol, 0.37 mL) was added slowly to the reaction mixture and stirred at 22° C. for 3 hrs. The reaction mixture was filtered and purified by column chromatography using a gradient of EtOAc in hexane containing 0.2% triethylamine to yield 22 (0.31 g, 65.3% yield) as white solid. ¹H NMR (400 MHz, CD3CN) δ 8.94 (s, 1H), 7.76 (dd, J=8.9, 8.2 Hz, 1H), 5.88 (dd, J=7.3, 4.6 Hz, 1H), 5.65 (dd, J=8.2, 3.0 Hz, 1H), 4.50-4.14 (m, 2H), 4.08-3.98 (m, 1H), 3.92-3.74 (m, 5H), 3.65 (dtd, J=10.3, 6.8, 4.7 Hz, 2H), 3.56-3.36 (m, 6H), 3.21 (d, J=10.2 Hz, 2H), 2.75-2.51 (m, 4H), 1.28-1.10 (m, 17H) ppm. ¹³C NMR (126 MHz, CD₃CN) δ 163.91, 163.88, 151.44, 151.40, 141.00, 140.98, 119.62, 119.60, 102.80, 102.72, 88.43, 87.92, 83.54, 83.51, 83.25, 83.23, 83.18, 83.14, 82.73, 82.69, 72.16, 72.03, 71.62, 71.48, 70.97, 70.89, 66.85, 66.83, 59.80, 59.65, 59.23, 59.08, 58.92, 58.90, 58.58, 58.56, 57.25, 57.02, 49.50, 44.21, 44.17, 44.11, 44.07, 27.25, 25.00, 24.98, 24.93, 24.89, 24.87, 24.84, 21.08, 21.02, 20.97 ppm. ³¹P NMR (202 MHz, CD₃CN) δ 151.48, 151.16 ppm HRMS calc. for C₂₃H₃₈N₅O₈PNa [M+Na]⁺ 566.2356, found 566.2379.

### 1-[(2R,5R)-4-hydroxyl-3-methoxy-5-[[methyl(tetrahydropyran-4-yl)amino]oxymethyl]tetra hydrofuran-2-yl]pyrimidine-2,4-dione ELN0132-275

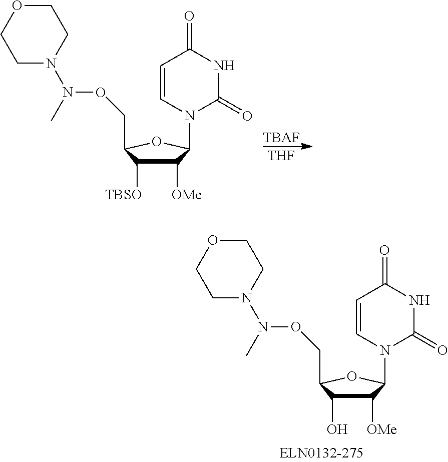

To a solution of 1-[(2R,5R)-4-[tert-butyl(dimethyl)silyl]oxy-3-methoxy-5-[[methyl (tetrahydropyran-4-yl)amino]oxymethyl]tetrahydrofuran-2-yl]pyrimidine-2,4-dione (0.45 g, 926.60 μmol) in THF (15 mL) at 22° C., tetrabutylammonium fluoride (318.13 mg, 1.20 mmol) was added slowly in single portion and then stirred for 12 hr. Volatile matters were removed in high vacuum pump and crude residue thus obtained was purified by flash column chromatography (gradient: 0-10% MeOH in DCM) to afford ELN0132-275 (0.31 g, 90% yield) as white solid. ¹H NMR (400 MHz, DMSO) δ 11.36 (d, J=2.2 Hz, 1H), 7.75 (d, J=8.1 Hz, 1H), 5.83 (d, J=4.8 Hz, 1H), 5.67 (dd, J=8.1, 2.2 Hz, 1H), 5.26 (d, J=6.1 Hz, 1H), 4.12-4.00 (m, 1H), 3.96 (td, J=4.8, 3.2 Hz, 1H), 3.86 (ddt, J=11.0, 5.8, 2.8 Hz, 3H), 3.82-3.69 (m, 2H), 3.36 (s, 3H), 3.34-3.22 (m, 2H), 3.17 (d, J=5.2 Hz, 1H), 2.63 (tt, J=10.9, 3.9 Hz, 1H), 2.53 (s, 3H), 1.75 (s, 2H), 1.40 (qd, J=12.1, 4.5 Hz, 2H) ppm. ¹³C NMR (101 MHz, DMSO) δ 163.03, 150.43, 140.24, 101.82, 86.39, 82.23, 82.08, 71.86, 68.64, 65.75, 63.29, 57.58, 48.60, 41.30, 29.46 ppm. HRMS calc. for C₁₆H₂₆N₃O₇ [M+H]⁺ 372.1771, found 372.1762.

### 1-[(2R,5R)-4-[tert-butyl(dimethyl)silyl]oxy-5-[[[(2E)-3,7-dimethylocta-2,6-dienylidene]amino] oxymethyl]-3-methoxy-tetrahydrofuran-2-yl]pyrimidine-2,4-dione ELN0132-58

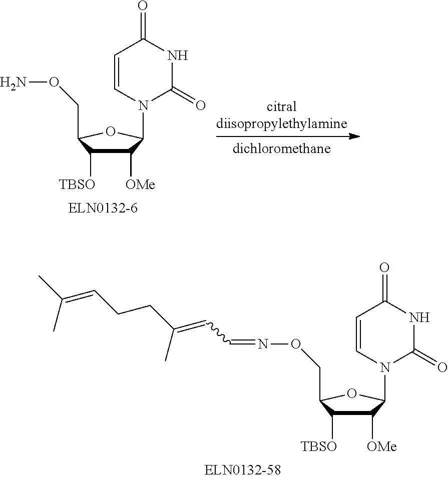

To a solution of ELN0132-6 (0.6 g, 1.55 mmol) in dry DCM (20 mL), diisopropylethylamine (606.40 mg, 4.65 mmol, 817.25 μL, 99% purity) was added and stirred for 5 minutes. To the resulting solution, citral (294.64 mg, 1.86 mmol, 331.80 μL) was added in single portion and the reaction mixture was stirred for 16 hrs at 25° C. TLC showed consumption of starting material. All the volatile matters were removed, diluted with EtOAc (30 mL) and washed with water (30 mL) and brine (2×30 mL). Organic layer was separated, dried over anhydrous Na₂SO₄, filtered and the filtrate was evaporated to dryness. The residue thus obtained was purified flash column chromatography to afford ELN0132-58 (0.62 g, 77% yield). ¹H NMR (400 MHz, CDCl₃) δ 10.11 (s, 1H), 7.97 (dd, J=12.6, 10.4 Hz, 1H), 7.83-7.71 (m, 1H), 7.33-7.29 (m, OH), 6.30 (ddt, J=9.2, 6.7, 1.5 Hz, 1H), 5.91-5.82 (m, 2H), 5.64 (dd, J=8.2, 4.5 Hz, 1H), 5.56 (dd, J=8.1, 7.1 Hz, 1H), 5.04 (dtdq, J=6.9, 4.1, 2.8, 1.4 Hz, 1H), 4.57-4.39 (m, 1H), 4.33-4.12 (m, 4H), 3.62 (ddd, J=15.5, 4.3, 2.0 Hz, 1H), 3.56-3.50 (m, 3H), 2.34-2.03 (m, 4H), 1.89-1.75 (m, 3H), 1.69-1.60 (m, 4H), 1.57 (dd, J=3.8, 1.5 Hz, 4H), 0.88 (s, 9H), 0.07 (d, J=1.6 Hz, 7H) ppm. ¹³C NMR (101 MHz, CDCl₃) δ 164.00, 163.96, 163.93, 163.87, 151.84, 151.63, 150.40, 150.38, 149.04, 148.94, 148.34, 148.09, 145.32, 145.00, 140.13, 140.09, 139.87, 139.83, 133.03, 132.88, 132.60, 132.39, 123.22, 122.99, 122.92, 122.81, 118.28, 117.34, 113.77, 112.95, 101.93, 101.91, 101.84, 88.59, 88.57, 88.14, 88.11, 84.03, 83.98, 83.76, 82.71, 82.65, 82.63, 77.48, 77.16, 76.84, 71.59, 71.52, 71.49, 69.51, 69.46, 69.41, 69.29, 58.50, 40.51, 40.12, 32.88, 32.75, 26.87, 26.78, 26.23, 26.15, 25.77, 25.75, 25.72, 24.72, 24.38, 18.21, 18.18, 17.79, 17.76, 17.37, 17.25, -4.63, −4.69, −4.80, −4.84, −4.85 ppm. HRMS calc. for C₂₆H₄₄N₃O₆Si [M+H]⁺ 522.2999, found 522.3007.

### 1-[(2R,5R)-5-[[bis[(2E)-3,7-dimethylocta-2,6-dienyl]amino]oxymethyl]-4-[tert-butyl(dimethyl) silyl]oxy-3-methoxy-tetrahydrofuran-2-yl]pyrimidine-2,4-dione ELN0132-84

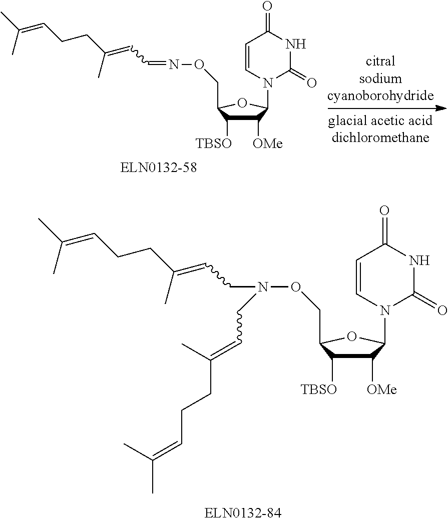

To a solution of ELN0132-58 (0.6 g, 1.15 mmol) in glacial acetic acid (5 mL), sodium cyanoborohydride (2.6 equiv) was added at 15° C. and stirred for 1 h. To the clear solution, citral (357.29 mg, 2.30 mmol, 402.35 μL) was added and stirred for 0.5 h and continued stirring for 30 minutes at 20° C. Second portion of sodium cyanoborohydride (2.6 equiv) was then added to this reaction mixture and stirred for 2 hr. TLC showed completion of reaction. After diluting the reaction mixture with DCM (20 mL), water (20 mL) was added, and organic layer was separated. DCM layer dried over anhydrous CaCl₂), filtered and filtrate was evaporated to dryness. Crude residue thus obtained was purified by flash column chromatography (gradient: 10-50% EtOAc in hexane) to afford ELN0132-84 (0.58 g, 878.83 mol, 76.42% yield). ¹³C NMR (101 MHz, CDCl₃) δ 163.62, 150.33, 140.43, 140.40, 139.78, 139.76, 139.66, 132.07, 131.82, 124.02, 124.00, 123.96, 120.27, 120.25, 119.41, 101.89, 101.83, 87.81, 84.03, 84.01, 83.98, 83.03, 82.98, 77.48, 77.16, 76.84, 71.17, 71.06, 69.99, 69.92, 58.36, 58.33, 55.55, 55.42, 55.26, 55.22, 39.90, 32.51, 26.65, 26.63, 26.60, 25.83, 25.79, 23.76, 23.74, 18.24, 17.80, 17.78, 16.71, 16.67, −4.46, −4.70 ppm. HRMS calc. for C₃₇H₆₆N₃O₆Si [M+H]⁺ 676.4721, found 676.4780.

### 1-[(2R,5R)-5-[[bis[(2E)-3,7-dimethylocta-2,6-dienyl]amino]oxymethyl]-4-hydroxyl-3-methoxy-tetrahydrofuran-2-yl]pyrimidine-2,4-dione ELN0132-98

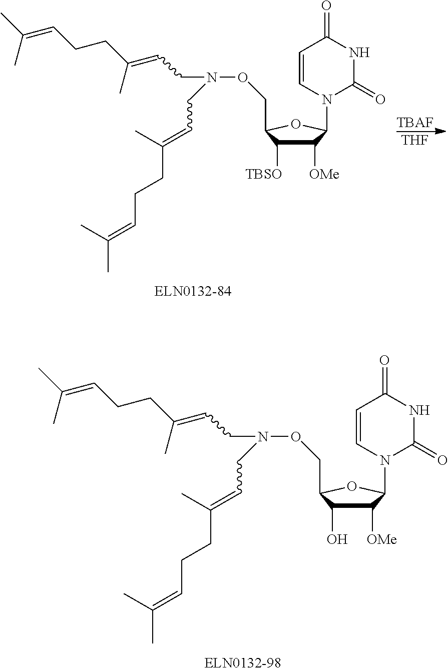

To a solution of ELN0132-84 (0.5 g, 757.61 mol) in THF (20 mL) at 25° C., tetrabutylammonium fluoride (300.13 mg, 1.14 mmol) was added slowly in single portion and then stirred for 16 hr. Volatile matters were removed in high vacuum pump and crude residue thus obtained was purified by flash column chromatography (gradient: 10-60% EtOAc in hexane) to afford (0.31 g, 75% yield). ¹H NMR (500 MHz, CDCl₃) δ 9.42-9.38 (m, 1H), 8.00-7.90 (m, 1H), 5.97-5.92 (m, 1H), 5.68 (dt, J=8.1, 1.5 Hz, 1H), 5.35-5.28 (m, 2H), 5.12-5.05 (m, 2H), 4.21-4.08 (m, 2H), 4.03 (dq, J=5.5, 2.7 Hz, 1H), 3.94 (ddd, J=11.0, 5.1, 2.7 Hz, 1H), 3.74 (dd, J=5.2, 2.4 Hz, 1H), 3.60 (s, 2H), 3.37 (t, J=5.7 Hz, 4H), 2.71 (dd, J=7.9, 3.1 Hz, 1H), 2.13-1.99 (m, 7H), 1.74 (d, J=1.5 Hz, 2H), 1.71-1.64 (m, 10H), 1.60 (dd, J=5.0, 1.3 Hz, 6H) ppm. ¹³C NMR (126 MHz, CDCl₃) δ 163.51, 163.49, 150.35, 140.20, 140.16, 139.87, 139.74, 139.72, 132.07, 131.80, 124.08, 123.96, 120.23, 119.48, 119.45, 102.00, 101.95, 87.36, 83.96, 83.94, 83.16, 71.30, 71.24, 68.82, 68.76, 60.52, 58.77, 55.86, 55.58, 39.85, 32.45, 26.65, 26.57, 26.54, 25.82, 25.79, 23.72, 23.71, 17.79, 16.68, 16.66 ppm. HRMS calc. for C₃₁H₅₂N₃O₆ [M+H]⁺ 562.3856, found 562.3866.

### 3-[[(2R,5R)-2-[[bis[(2E)-3,7-dimethylocta-2,6-dienyl]amino]oxymethyl]-5-(2,4-dioxopyrimidin-1-yl)-4-methoxy-tetrahydrofuran-3-yl]oxy-(diisopropylamino)phosphanyl] oxypropane nitrile ELN0132-107

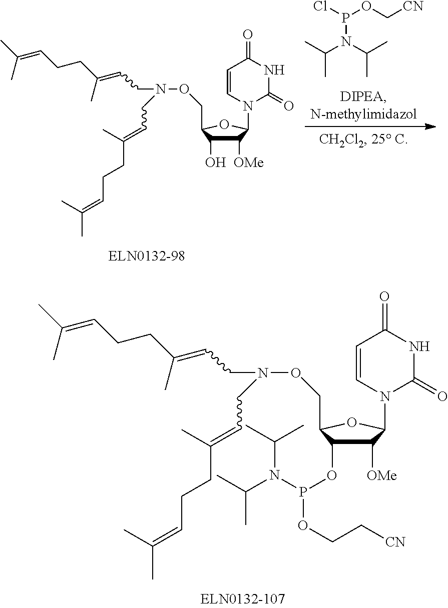

To a clear solution of ELN0132-98 (0.3 g, 549.74 mol) in DCM (10 mL), diisopropylethylamine (358.83 mg, 2.75 mmol, 483.60 μL) and N-methylimidazole (159.56 mg, 1.92 mmol, 154.92 μL) were added at 25° C. To this reaction mixture, 2-cyanoethyl-N,N-diisopropylchlorophosphoramidite (273.92 mg, 1.10 mmol, 258.42 μL, 95% purity) was added slowly after 5 minutes and stirred for 0.5 hr. Reaction mixture was diluted with DCM (20 mL) and quenched with 10% NaHCO₃ solution (20 mL). Organic layer was separated, dried on anhydrous Na₂SO₄, filtered and filtrate was evaporated to dryness. The crude compound was thus obtained was purified by flash column chromatography (gradient: 10-50% EtOAc in hexane) to afford ELN0132-107 (0.32 g, 78% yield). ¹H NMR (400 MHz, CDCl₃) δ 8.44 (s, 1H), 7.97-7.81 (m, 1H), 6.05-5.96 (m, 1H), 5.67 (dd, J=8.2, 2.6 Hz, 1H), 5.36-5.25 (m, 1H), 5.08 (dddp, J=5.5, 4.1, 2.8, 1.4 Hz, 2H), 4.31 (dddd, J=17.1, 14.6, 8.4, 4.8 Hz, 1H), 4.17-4.04 (m, 1H), 3.97-3.76 (m, 3H), 3.73-3.55 (m, 1H), 3.54-3.45 (m, 3H), 3.45-3.34 (m, 4H), 2.73-2.57 (m, 2H), 2.05 (q, J=8.0 Hz, 9H), 1.77-1.52 (m, 19H), 1.35-1.14 (m, 13H) ppm. ³¹P NMR (162 MHz, CDCl₃) δ 152.28, 152.25, 152.12, 152.07, 152.00 ppm. HRMS calc. for C₄₀H₆₉N₅O₇P [M+H]⁺ 762.4935, found 762.4915.

### 1-[(2R,5R)-4-[tert-butyl(dimethyl)silyl]oxy-5-[(decylideneamino)oxymethyl]-3-methoxy-tetra hydrofuran-2-yl]pyrimidine-2,4-dione ELN0132-42

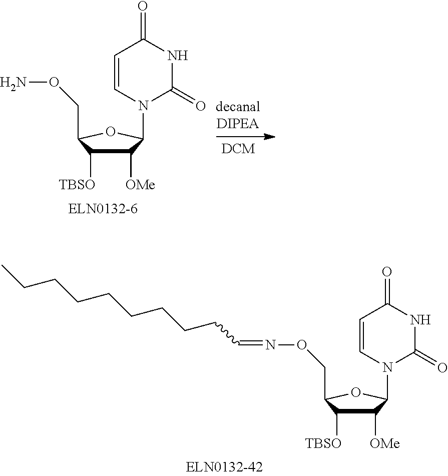

To a solution of 1-[(2R,5R)-5-(aminooxymethyl)-4-[tert-butyl(dimethyl)silyl]oxy-3-methoxy-tetrahydrofuran-2-yl]pyrimidine-2,4-dione (0.2 g, 0.52 mmol) in dry DCM (20 mL), DIPEA (0.27 mL, 1.55 mmol,) was added and stirred for 5 minutes. To the resulting solution, decanal (0.16 g, 1.03 mmol) was added in single portion and the reaction mixture was stirred for 16 h at 25° C. All the volatile matters were removed when TLC showed consumption of starting material. Residue was diluted with EA (30 mL) and washed with DI water (30 mL) and brine (2×30 mL). Organic layer was separated, dried over anhydrous Na₂SO₄, filtered and the filtrate was evaporated to dryness. The crude residue thus obtained was purified combiflash column chromatography to afford ELN0132-42 (0.26 g, 96% yield). ¹H NMR (500 MHz, CDCl₃, TMS, 25° C.) δ 10.38-10.12 (m, 1H), 7.74 (dd, J=24.0, 8.1 Hz, 1H), 7.35 (t, J=6.2 Hz, 0.5H), 6.67 (t, J=5.4 Hz, 0.5H), 5.84 (dd, J=6.5, 2.0 Hz, 1H), 5.63 (ddd, J=13.2, 8.2, 1.5 Hz, 1H), 4.43 (ddd, J=44.8, 12.4, 2.3 Hz, 1H), 4.27-4.11 (m, 3H), 3.66-3.55 (m, 1H), 3.52 (d, J=6.2 Hz, 3H), 2.25 (tt, J=7.4, 5.3 Hz, 1H), 2.16 (dt, J=7.8, 6.4 Hz, 1H), 1.54-1.41 (m, 2H), 1.33-1.12 (m, 12H), 0.86 (bs, 9H), 0.83 (t, J=6.9 Hz, 3H), 0.11-0.01 (m, 6H) ppm. ¹³C NMR (101 MHz, CDCl₃, 25° C.) δ 164.03, 163.97, 152.64, 151.85, 150.39, 150.37, 140.02, 139.71, 101.89, 101.81, 88.46, 88.31, 83.87, 83.74, 82.55, 82.47, 71.40, 71.04, 69.41, 69.24, 60.37, 58.49, 58.45, 31.86, 31.85, 29.45, 29.43, 29.39, 29.36, 29.29, 29.26, 29.13, 26.65, 26.24, 26.02, 25.71, 25.70, 22.66, 18.14, 14.11, −4.69, −4.74, −4.87, −4.94 ppm. HRMS calc. for C₂₆H₄₈N₃O₆Si [M+H]⁺ 526.3312, found 526.3314.

### 1-[(2R,5R)-5-[(didecylamino)oxymethyl]-4-hydroxyl-3-methoxy-tetrahydrofuran-2-yl]pyrimidine-2,4-dione ELN0132-55

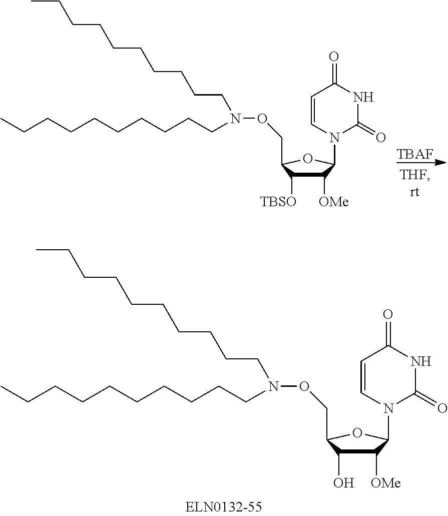

To a clear solution of 1-[(2R,5R)-4-[tert-butyl(dimethyl)silyl]oxy-5-[(didecylamino)oxymethyl]-3-methoxy-tetrahydrofuran-2-yl]pyrimidine-2,4-dione (0.51 g, 0.76 mmol) in tetrahydrofuran (10 mL), TBAF (0.24 g, 0.92 mmol) was slowly added and stirred at 20° C. for 16 h. Volatile matters were evaporated when TLC showed completion of the reaction. The crude mass thus obtained was purified by combiFlash column chromatography (Gradient: 0-70% EA in hexane) to afford ELN0132-55 (0.21 g, 50% yield). ¹H NMR (500 MHz, CDCl₃, TMS, 25° C.) δ 8.75 (s, 1H), 8.00 (d, J=8.1 Hz, 1H), 5.95 (d, J=2.3 Hz, 1H), 5.68 (d, J=8.1 Hz, 1H), 4.22 (td, J=7.4, 5.1 Hz, 1H), 4.13 (dd, J=11.0, 2.3 Hz, 1H), 4.03 (dt, J=7.1, 2.5 Hz, 1H), 3.93 (dd, J=11.0, 2.7 Hz, 1H), 3.74 (dd, J=5.2, 2.3 Hz, 1H), 3.61 (s, 2H), 2.67 (qd, J=9.6, 8.2, 3.5 Hz, 5H), 1.55 (p, J=7.2 Hz, 4H), 1.36-1.18 (m, 29H), 0.88 (t, J=6.9 Hz, 5H) ppm. ¹³C NMR (101 MHz, CDCl₃, 25° C.) δ 163.08, 150.16, 140.13, 101.89, 87.39, 83.93, 83.22, 71.34, 68.68, 59.48, 58.81, 32.05, 29.75, 29.72, 29.70, 29.47, 27.65, 27.26, 22.83, 14.25 ppm. HRMS calc. for C₃₀H₅₆N₃O₆ [M+H]⁺554.4169, found 554.4178.

### 3-[[(2R,5R)-2-[(didecylamino)oxymethyl]-5-(2,4-dioxopyrimidin-1-yl)-4-methoxy-tetrahydrofuran-3-yl]oxy-(diisopropylamino)phosphanyl]oxypropanenitrile ELN0132-72

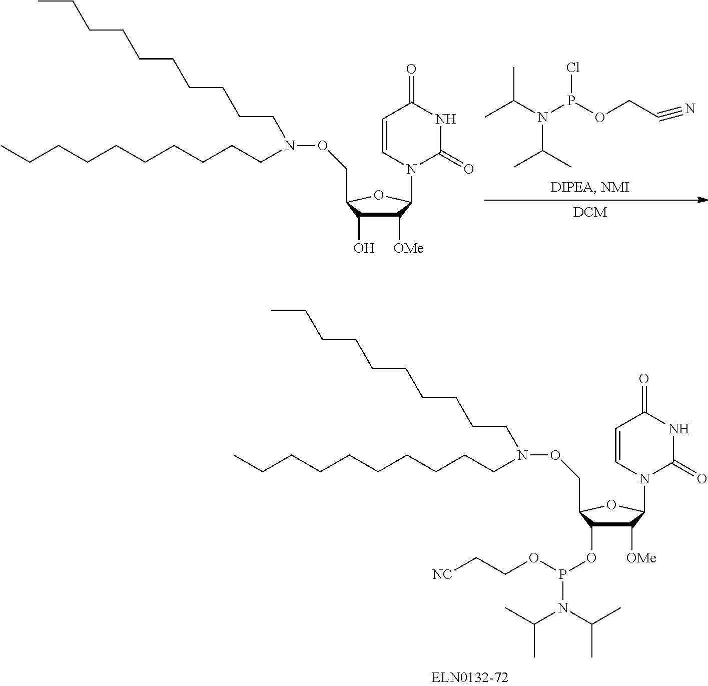

To a clear solution of ELN0132-55 (0.2 g, 0.36 mmol) in DCM (10 mL), was added DIPEA (0.28 g, 2.17 mmol, 0.38 mL) and NMI (0.104 g, 1.26 mmol, 0.10 mL) at 22° C. The reaction mixture was stirred for 5 minutes and 2-cyanoethyl-N,N-diisopropylchlorophosphoramidite (0.171 g, 0.72 mmol, 0.16 mL) was added in single portion. Stirring was continued for 1.25 h at 22° C. after which the reaction mixture was diluted with DCM (20 mL) and saturated NaHCO₃ solution (20 mL) was added. Organic layer was washed with brine (30 mL), separated, dried over anhydrous Na₂SO₄ and filtered. The filtrate was evaporated to dryness and the crude mass thus obtained, was purified by flash column chromatography to afford ELN0132-72 (0.22 g, 81% yield) as transparent gum. ¹H NMR (500 MHz, CDCl₃, TMS, 25° C.) δ 8.34 (s, 1H), 7.94 (dd, J=27.9, 8.2 Hz, 1H), 5.99 (dd, J=4.0, 2.3 Hz, 1H), 5.67 (dd, J=8.2, 3.0 Hz, 1H), 4.37-4.24 (m, 1H), 4.19 (dt, J=5.2, 2.5 Hz, 1H), 4.10-4.03 (m, 1H), 3.97-3.79 (m, 4H), 3.77-3.58 (m, 2H), 3.51 (d, J=9.8 Hz, 3H), 2.75-2.56 (m, 6H), 1.62-1.48 (m, 5H), 1.34-1.13 (m, 48H), 0.88 (t, J=6.9 Hz, 6H) ppm. ¹³C NMR (101 MHz, CDCl₃, 25° C.) δ ppm. 31p NMR (162 MHz, CDCl₃, 25° C.) δ 151.93, 151.89 ppm. HRMS calc. for C₃₉H₇₃N₅O₇P [M+H]⁺ 754.5248, found 754.5247.

### 1-[(2R,5R)-4-[tert-butyl(dimethyl)silyl]oxy-5-[(hexadecylideneamino)oxymethyl]-3-methoxy-tetrahydrofuran-2-yl]pyrimidine-2,4-dione ELN0132-52

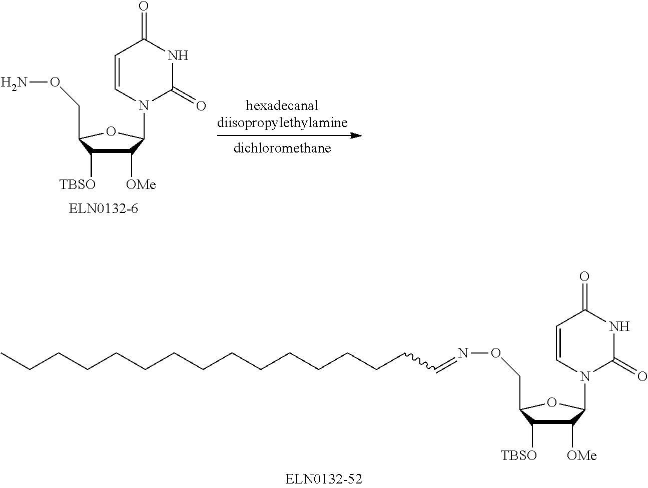

To a solution of ELN0132-6 (1.0 g, 2.58 mmol) in dry DCM (20 mL), diisopropylethylamine (1.00 g, 7.74 mmol, 1.35 mL) was added and stirred for 5 minutes. To the resulting solution, Hexadecanal (620.44 mg, 2.58 mmol) was added in single portion and the reaction mixture was stirred for 16 hr at 25° C. TLC showed consumption of starting material. All the volatile matters were removed, diluted with EA (30 mL) and washed with DI water (30 mL) and brine (2×30 mL). Organic layer was separated, dried over anhydro Na₂SO₄, filtered and the filtrate was evaporated to dryness. The residue thus obtained was purified flash column chromatography to afford ELN0132-52 (1.34 g, 85% yield) as mixture of E and Z isomers. ¹H NMR (500 MHz, CDCl₃) δ 9.15 (s, 2H), 7.79 (d, J=8.2 Hz, 1H), 7.74 (d, J=8.1 Hz, 1H), 7.38 (t, J=6.2 Hz, 1H), 6.70 (t, J=5.4 Hz, 1H), 5.87 (dd, J=6.0, 2.2 Hz, 2H), 5.66 (dd, J=13.0, 8.2 Hz, 2H), 4.50 (dd, J=12.4, 2.2 Hz, 1H), 4.41 (dd, J=12.3, 2.4 Hz, 1H), 4.26 (dd, J=12.4, 2.4 Hz, 1H), 4.23-4.15 (m, 6H), 3.67-3.60 (m, 2H), 3.55 (d, J=5.9 Hz, 6H), 2.29 (tdd, J=7.4, 5.4, 4.2 Hz, 2H), 2.23-2.15 (m, 2H), 1.51-1.44 (m, 4H), 1.25 (s, 50H), 0.90 (s, 18H), 0.87 (d, J=7.1 Hz, 6H), 0.10-0.07 (m, 12H) ppm. ¹³C NMR (101 MHz, CDCl₃) δ 163.43, 163.38, 152.81, 152.03, 150.19, 150.16, 140.11, 139.82, 102.00, 101.93, 88.53, 88.40, 84.01, 83.88, 82.79, 82.73, 71.61, 71.24, 69.59, 69.43, 58.63, 58.59, 32.07, 29.84, 29.82, 29.80, 29.76, 29.65, 29.63, 29.58, 29.54, 29.51, 29.50, 29.44, 29.29, 26.80, 26.38, 26.17, 25.83, 22.83, 18.28, 14.25, −4.55, −4.60, −4.72, -4.79 ppm. HRMS calc. for C₃₂H₆₀N₃O₆Si [M+H]⁺ 610.4251, found 610.4243.

### 1-[(2R,5R)-5-[(hexadecylideneamino)oxymethyl]-4-hydroxyl-3-methoxy-tetrahydrofuran-2-yl]pyrimidine-2,4-dione ELN0132-81

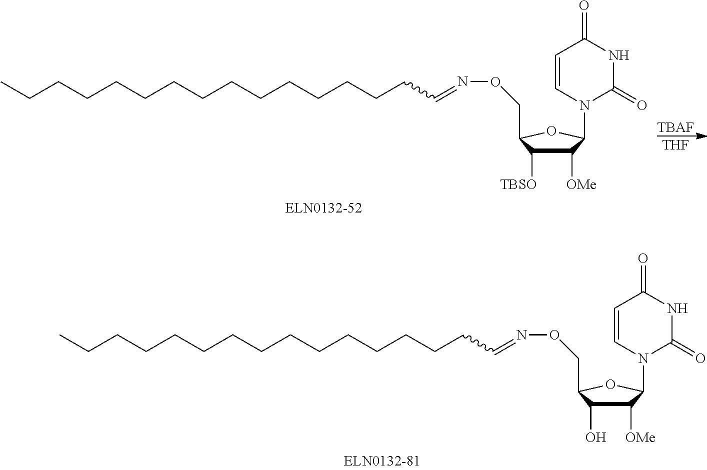

To a solution of ELN0132-52 (1 g, 1.64 mmol) in THF (15 mL) at 25° C., tetrabutylammonium fluoride (433.02 mg, 1.64 mmol) was added slowly in single portion and then stirred for 12 h. Volatile matters were removed in high vacuum pump and crude residue thus obtained was purified by combiflash chromatography (Gradient: 20-60% EA in hexane) to afford ELN0132-81 (0.75 g, 92% yield). ¹H NMR (500 MHz, CDCl₃, TMS, 25° C.) δ 9.04 (d, J=10.5 Hz, 1H), 7.73 (dd, J=19.8, 8.1 Hz, 1H), 7.40 (t, J=6.2 Hz, 0.5H), 6.71 (t, J=5.4 Hz, 0.5H), 5.93 (t, J=2.3 Hz, 1H), 5.68 (ddd, J=11.0, 8.1, 1.7 Hz, 1H), 4.48 (ddd, J=36.9, 12.5, 2.3 Hz, 1H), 4.32 (ddd, J=31.7, 12.6, 3.1 Hz, 1H), 4.21 (tdd, J=7.9, 5.2, 2.3 Hz, 1H), 4.12 (ddt, J=7.8, 5.6, 2.7 Hz, 1H), 3.75 (ddd, J=9.2, 5.2, 2.2 Hz, 1H), 3.62 (d, J=5.5 Hz, 3H), 2.72 (dd, J=8.3, 5.1 Hz, 1H), 2.30 (td, J=7.6, 5.4 Hz, 1H), 2.22-2.13 (m, 1H), 1.47 (q, J=7.2 Hz, 2H), 1.25 (s, 25H), 0.87 (t, J=6.9 Hz, 3H) ppm. ¹³C NMR (101 MHz, CDCl₃, 25° C.) δ 163.16, 163.12, 153.12, 152.30, 150.16, 150.13, 139.80, 139.57, 102.25, 102.23, 87.80, 87.69, 83.87, 83.74, 83.25, 83.23, 72.09, 71.78, 68.87, 68.70, 58.90, 58.88, 32.07, 29.84, 29.82, 29.80, 29.75, 29.65, 29.63, 29.57, 29.50, 29.43, 29.30, 26.78, 26.35, 26.14, 22.83, 14.25 ppm. HRMS calc. for C₂₆H₄₆N₃O₆ [M+H]⁺ 496.3387, found 496.3389.

3-[(diisopropylamino)-[(2R,5R)-5-(2,4-dioxopyrimidin-1-yl)-2-[(hexadecylideneamino)oxy methyl]-4-methoxy-tetrahydrofuran-3-yl]oxy-phosphanyl]oxypropanenitrile ELN0132-92:

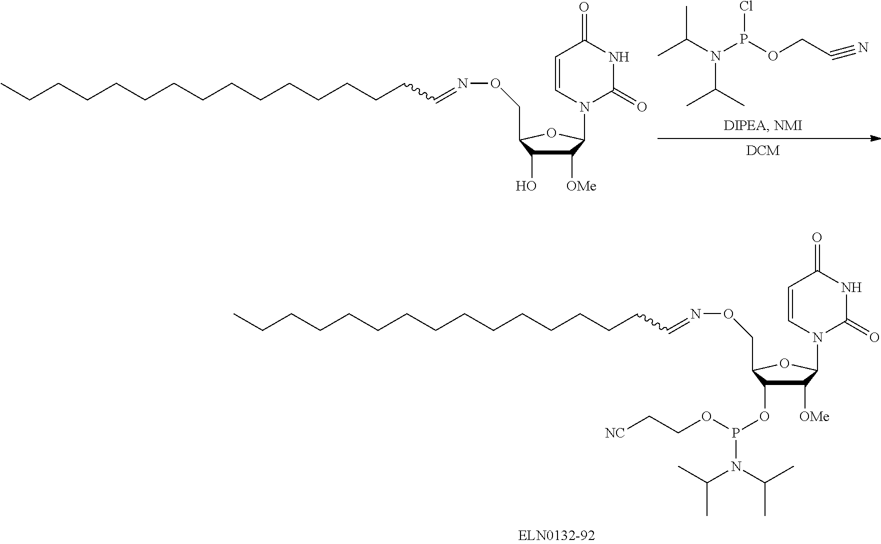

To a clear solution of ELN0132-81 (0.7 g, 1.41 mmol) in DCM (15 mL), DIPEA (0.92 g, 7.06 mmol, 1.24 mL) and N-methylimidazole (0.41 g, 4.94 mmol, 0.40 mL) were added at 25° C. To this reaction mixture, was added 2-cyanoethyl-N,N-diisopropylchlorophosphoramidite (0.704 g, 2.82 mmol, 0.66 mL) slowly after 5 minutes and stirred for 1.5 h. Reaction mixture was diluted with DCM (20 mL) and quenched with 10% NaHCO₃ solution (20 mL). Organic layer was separated, dried on anhydrous Na₂SO₄, filtered and filtrate was evaporated to dryness. The crude compound was thus obtained was purified by combiflash chromatography (gradient: 10-50% EtOAc in hexane) to afford ELN0132-92 (0.75 g, 76% yield) as transparent gum. ¹H NMR (400 MHz, CDCl₃, TMS, 25° C.) δ 9.15 (s, 1H), 7.72 (ddd, J=15.7, 8.2, 5.7 Hz, 1H), 7.41 (dt, J=10.9, 6.2 Hz, 0.5H), 6.71 (dt, J=7.1, 5.3 Hz, 0.5H), 5.97 (dd, J=6.5, 3.3 Hz, 1H), 5.77-5.48 (m, 1H), 4.56-4.18 (m, 4H), 4.00-3.70 (m, 3H), 3.68-3.59 (m, 2H), 3.55 (d, J=4.0 Hz, 1.6H), 3.51 (s, 1.4H), 2.74-2.55 (m, 2H), 2.38-2.14 (m, 2H), 1.48 (qd, J=7.2, 4.7, 2.9 Hz, 2H), 1.33-1.13 (m, 38H), 0.95-0.69 (m, 3H) ppm. ¹³C NMR (101 MHz, CDCl₃, 25° C.) δ ppm. ³¹P NMR (162 MHz, CDCl₃, 25° C.) δ 151.54, 151.27, 150.90 ppm. HRMS calc. for C₃₅H₆₃N₅O₇P [M+H]⁺ 696.4465, found 696.4449.

### 1-[(2R,5R)-4-[tert-butyl(dimethyl)silyl]oxy-5-[(dihexadecylamino)oxymethyl]-3-methoxy-tetrahydrofuran-2-yl]pyrimidine-2,4-dione ELN0132-57

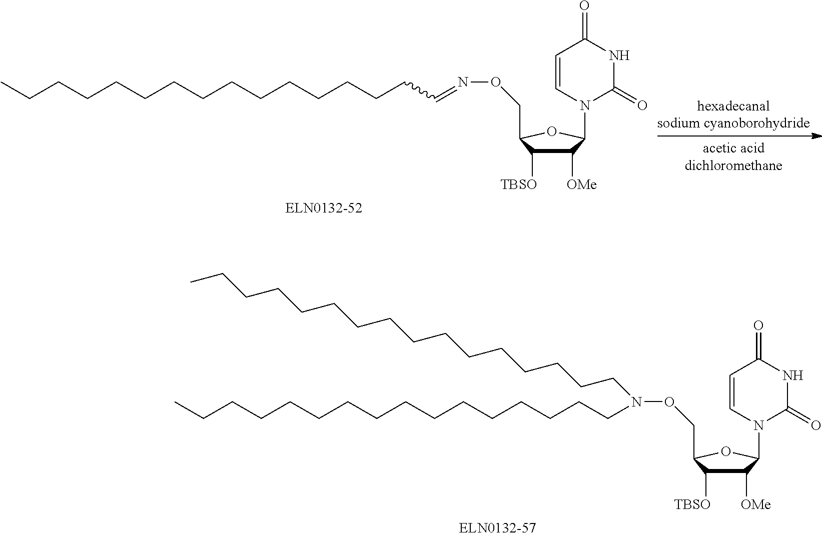

To a solution of ELN0132-52 (0.12 g, 196.75 μmol) in glacial acetic acid (0.3 mL) was added sodium cyanoborohydride (33.84 mg, 511.55 μmol) under 15° C. The reaction mixture was stirred for 1 hr at 15° C. and hexadecanal (141.91 mg, 590.25 μmol) in DCM (0.2 mL) was added. The stirring was continued for 30 min and additional amount of sodium cyanoborohydride (33.84 mg, 511.55 μmol) was added. The resulting mixture was stirred for another 2 hr and then diluted with DCM (10 mL) and washed with ice water. The organic layer was separated, dried over anhydrous Na₂SO₄ and concentrated. The crude material was purified by flash silica gel column chromatography (5% MeOH in DCM) to give ELN0132-57 (0.145 g, 88% yield) (R_{f}=0.4; developed in 30% EtOAc in hexane). ¹H NMR (400 MHz, CDCl₃) δ 9.03 (s, 1H), 8.03 (d, J=8.2 Hz, 1H), 5.92 (d, J=2.3 Hz, 1H), 5.67 (d, J=8.1 Hz, 1H), 4.16 (dd, J=6.9, 4.8 Hz, 1H), 4.12-4.04 (m, 2H), 3.90-3.84 (m, 1H), 3.68-3.57 (m, 1H), 3.52 (s, 3H), 2.73-2.62 (m, 4H), 1.53 (p, J=6.9 Hz, 5H), 1.25 (bs, 60H), 0.89 (d, J=11.9 Hz, 15H), 0.09 (d, J=5.4 Hz, 6H) ppm. ¹³C NMR (101 MHz, CDCl₃) δ 175.33, 163.48, 150.17, 140.47, 101.68, 87.90, 84.07, 82.93, 71.20, 69.64, 63.26, 59.31, 58.30, 32.97, 32.08, 29.86, 29.82, 29.78, 29.76, 29.71, 29.59, 29.51, 27.64, 27.27, 25.90, 25.84, 22.84, 20.68, 18.26, 14.26, −4.38, −4.72 ppm.

### 1-[(2R,5R)-5-[(dihexadecylamino)oxymethyl]-4-hydroxyl-3-methoxy-tetrahydrofuran-2-yl]pyrimidine-2,4-dione ELN0132-103

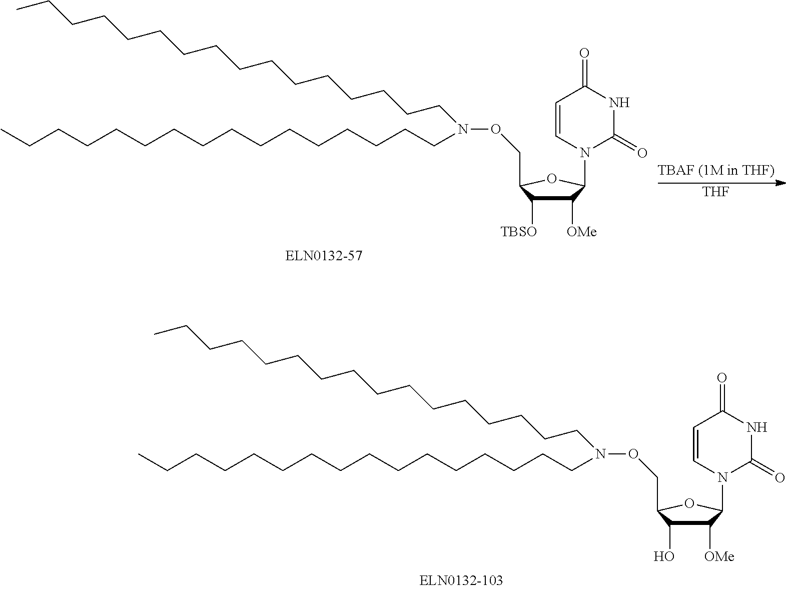

To a solution of ELN0132-57 (0.12 g, 143.48 mol) in THF (10 mL) at 25° C., Tetrabutylammonium fluoride, 1M in THF (56.84 mg, 215.22 mol, 62.95 μL, 99% purity) was added slowly in single portion and then stirred for 12 hr. Volatile matters were removed in high vacuum pump and crude residue thus obtained was purified by Combiflash chromatography (gradient: 10-60% EtOAc in hexane) to afford ELN0132-103 (0.091 g, 88% yield). ¹H NMR (500 MHz, CDCl₃) δ 8.55 (d, J=2.3 Hz, 1H), 7.99 (d, J=8.2 Hz, 1H), 5.95 (d, J=2.2 Hz, 1H), 5.68 (dd, J=8.1, 2.2 Hz, 1H), 4.22 (td, J=7.6, 5.2 Hz, 1H), 4.13 (dd, J=11.0, 2.3 Hz, 1H), 4.03 (dt, J=7.1, 2.5 Hz, 1H), 3.93 (dd, J=11.0, 2.7 Hz, 1H), 3.74 (dd, J=5.2, 2.3 Hz, 1H), 3.61 (s, 3H), 2.72-2.61 (m, 5H), 1.55 (p, J=7.2 Hz, 5H), 1.25 (s, 55H), 0.88 (t, J=6.9 Hz, 6H) ppm.

### 3-[[(2R,5R)-2-[(dihexadecylamino)oxymethyl]-5-(2,4-dioxopyrimidin-1-yl)-4-methoxy-tetra hydrofuran-3-yl]oxy-(diisopropylamino)phosphanyl]oxypropanenitrile ELN0132-108

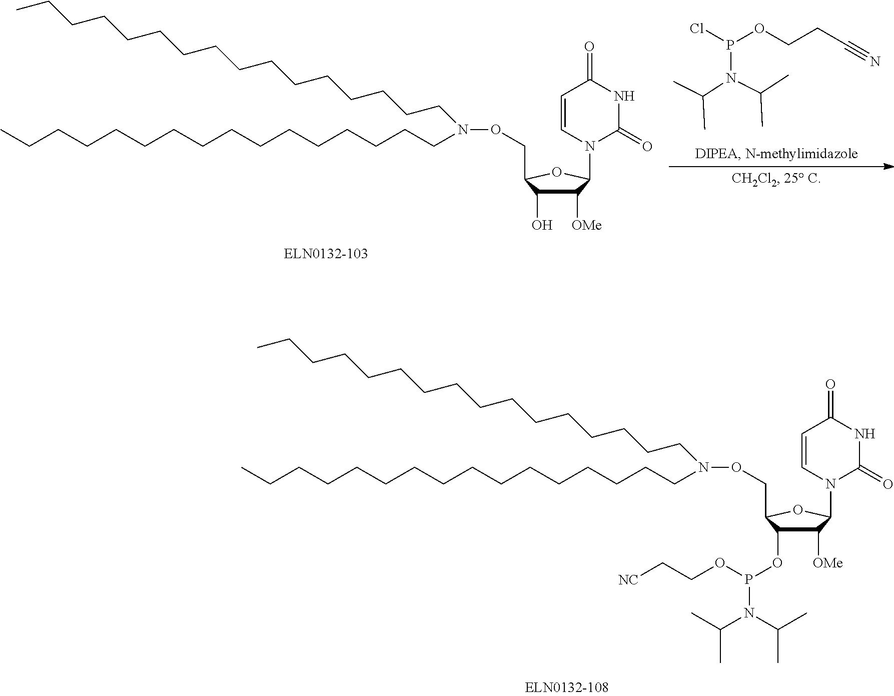

To a clear solution of ELN0132-103 (0.08 g, 110.79 mol) in DCM (10 mL), diisopropylethylamine (72.32 mg, 553.95 mol, 97.46 μL) and N-methylimidazole (32.16 mg, 387.76 mol, 31.22 μL) were added at 25° C. To this reaction mixture, 2-cyanoethyl-N,N-diisopropylchlorophosphoramidite (55.20 mg, 221.58 mol, 52.08 μL, 95% purity) was added slowly after 5 minutes and stirred for 1 hr. Reaction mixture was diluted with DCM (20 mL) and quenched with 10% NaHCO₃ solution (20 mL). Organic layer was separated, dried on anhydrous Na₂SO₄, filtered and filtrate was evaporated to dryness. The crude compound was thus obtained was purified by combiflash chromatography (gradient: 10-50% EtOAc in hexane) to afford ELN0132-108 (0.085 g, 83% yield). ¹H NMR (400 MHz, CDCl₃) δ 8.76 (s, 1H), 8.08-7.71 (m, 1H), 5.99 (t, J=3.6 Hz, 1H), 5.67 (dd, J=8.2, 2.7 Hz, 1H), 4.42-4.23 (m, 1H), 4.21-4.02 (m, 3H), 3.96-3.77 (m, 4H), 3.74-3.57 (m, 2H), 3.51 (d, J=6.6 Hz, 3H), 2.65 (ddd, J=17.1, 10.2, 6.2 Hz, 6H), 1.54 (q, J=7.3 Hz, 4H), 1.35-1.14 (m, 63H), 0.91-0.82 (m, 6H) ppm. ³¹P NMR (162 MHz, CDCl₃) δ 152.04, 151.98 ppm.

### Methyl-16-[[(2R,5R)-3-[tert-butyl(dimethyl)silyl]oxy-5-(2,4-dioxopyrimidin-1-yl)-4-methoxy-tetrahydrofuran-2-yl]methoxy-hexadecyl-amino]hexadecanoate ELN0132-176

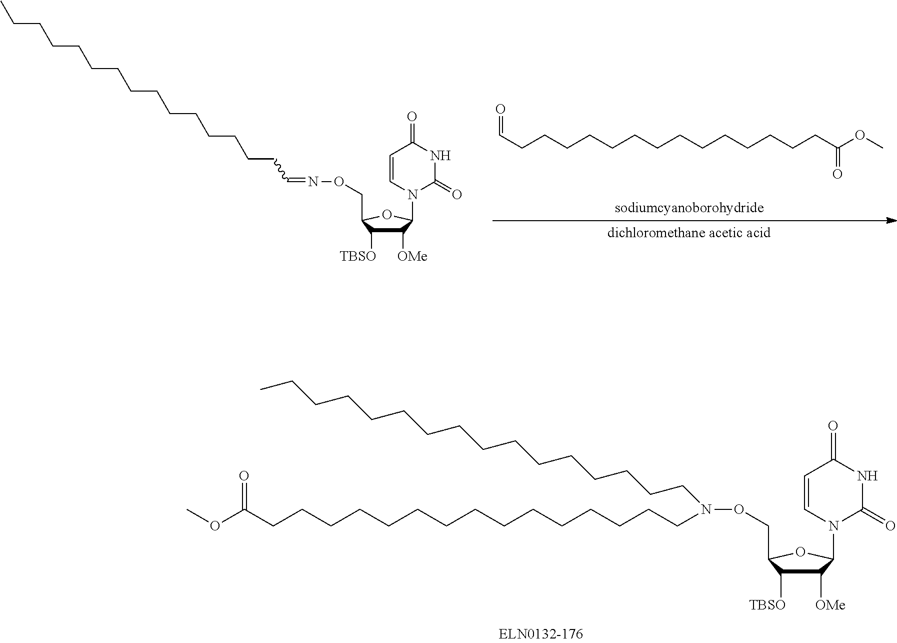

To a clear solution of ELN0132-52 (1.5 g, 2.46 mmol) in dry DCM (20 mL) and acetic acid (10 mL) was added sodium cyanoborohydride (410.02 mg, 6.39 mmol, 98% purity) in single portion and the reaction mixture was stirred for 1.5 hrs at 15° C. To the resulting mixture was added methyl 16-oxohexadecanoate (1.05 g, 3.69 mmol) and stirring was continued for 1 hr. Reaction was again cooled to 15° C. and sodium cyanoborohydride (410.02 mg, 6.39 mmol) was added. After 2.5 hrs TLC showed consumption of starting materials. Reaction mixture was diluted with DCM (25 mL) and quenched with water (30 mL). Layers were separated and aqueous layer was washed with DCM (20 mL). Combined DCM layer was washed with brine (2×30 mL). Organic layer was separated, dried over anhydrous Na₂SO₄, filtered and the filtrate was evaporated to dryness. The residue thus obtained was purified flash column chromatography (gradient: 10-40% EtOAc in hexane) to afford ELN0132-176 (1.95 g, 90% yield). ¹H NMR (400 MHz, CDCl₃) δ 8.96 (d, J=2.2 Hz, 1H), 8.02 (d, J=8.2 Hz, 1H), 5.92 (d, J=2.3 Hz, 1H), 5.68 (dd, J=8.2, 2.1 Hz, 1H), 4.17 (dd, J=6.9, 4.9 Hz, 1H), 4.12-4.03 (m, 2H), 3.92-3.82 (m, 1H), 3.67 (s, 6H), 3.65-3.59 (m, 1H), 3.53 (s, 3H), 2.73-2.62 (m, 4H), 2.30 (t, J=7.6 Hz, 3H), 1.68-1.48 (m, 6H), 1.25 (s, 63H), 0.89 (d, J=12.1 Hz, 12H), 0.10 (d, J=5.4 Hz, 6H) ppm. ¹³C NMR (101 MHz, CDCl₃) δ 174.50, 163.36, 150.18, 140.34, 101.70, 87.83, 84.03, 82.87, 71.18, 69.57, 63.21, 59.33, 58.29, 51.58, 34.25, 32.94, 32.06, 29.84, 29.81, 29.76, 29.74, 29.71, 29.70, 29.59, 29.57, 29.50, 29.40, 29.29, 27.62, 27.25, 25.88, 25.82, 25.09, 22.83, 18.24, 14.26, −4.40, −4.74 ppm. HRMS calc. for C₄₉H₉₄N₃O₈Si [M+H]⁺ 880.6810, found 880.6799.

### Methyl-16-((((2R,3R,4R,5R)-3-((tert-butyldimethylsilyl)oxy)-5-(2,4-dioxo-3,4-dihydropyrimid-in-1(2H)-yl)-4-methoxytetrahydrofuran-2-yl)methoxy)(hexadecyl)amino) hexadecanoate ELN0132-382

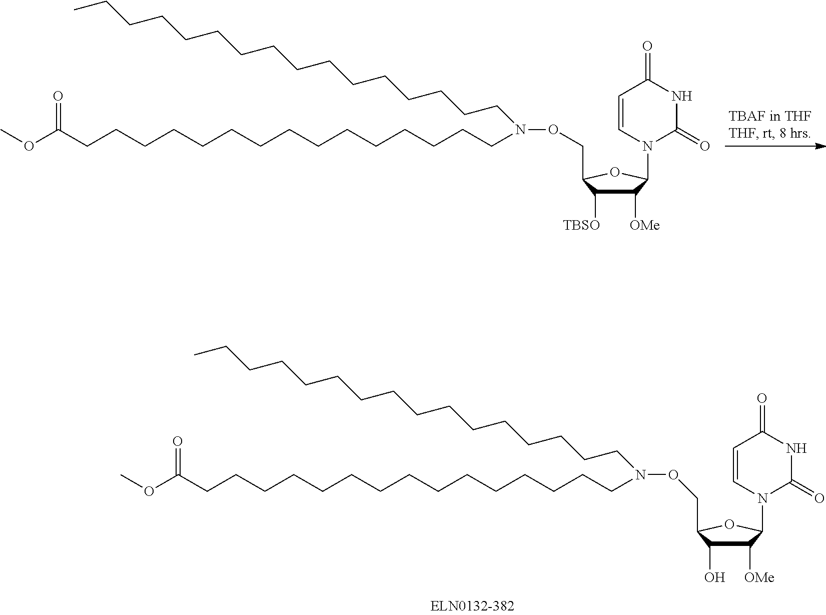

To a clear solution of ELN0132-176 (1.9 g, 2.16 mmol) in THF (30 mL) at 22° C., tetrabutylammonium fluoride (733.57 mg, 2.81 mmol) was added slowly in single portion and then stirred for 4 hr. All the volatile matters were removed under high vacuum pump and the residue thus obtained was purified by flash column chromatography (gradient: 20-60% EtOAc in hexane) to afford ELN0132-382 (1.35 g, 82% yield) as white semi-solid. ¹H NMR (600 MHz, CDCl₃) δ 8.76 (s, 1H), 8.00 (dd, J=8.4, 2.1 Hz, 1H), 5.95 (d, J=2.5 Hz, 1H), 5.71-5.65 (m, 1H), 4.22 (q, J=6.8 Hz, 1H), 4.13 (dd, J=11.1, 2.5 Hz, 1H), 4.03 (dd, J=7.2, 2.5 Hz, 1H), 3.93 (dd, J=11.1, 2.6 Hz, 1H), 3.74 (dd, J=5.2, 2.5 Hz, 1H), 3.67 (d, J=2.1 Hz, 3H), 3.61 (d J=2.1 Hz, 3H), 2.67 (q, J=7.2 Hz, 5H), 2.30 (td, J=7.6, 2.2 Hz, 2H), 1.64-1.58 (m, 3H), 1.54 (q, J=6.9 Hz, 5H), 1.35-1.22 (m, 43H), 0.88 (td, J=7.1, 2.1 Hz, 3H) ppm. ¹³C NMR (151 MHz, CDCl₃) δ 174.52, 163.11, 150.15, 140.14, 101.87, 87.35, 83.90, 83.20, 71.32, 68.63, 59.49, 58.80, 51.59, 34.26, 32.07, 29.85, 29.83, 29.81, 29.78, 29.76, 29.74, 29.70, 29.59, 29.51, 29.40, 29.29, 27.64, 27.25, 25.10, 22.83, 14.26 ppm. HRMS calc. for C₄₃H₈₀N₃O₈[M+H]⁺ 766.5945, found 766.5949.

### Methyl-16-[[(2R,5R)-3-[2-cyanoethoxy-(diisopropylamino)phosphanyl]oxy-5-(2,4-dioxopyrimidin-1-yl)-4-methoxy-tetrahydrofuran-2-yl]methoxy-hexadecyl-amino]hexadecanoate ELN0132-391

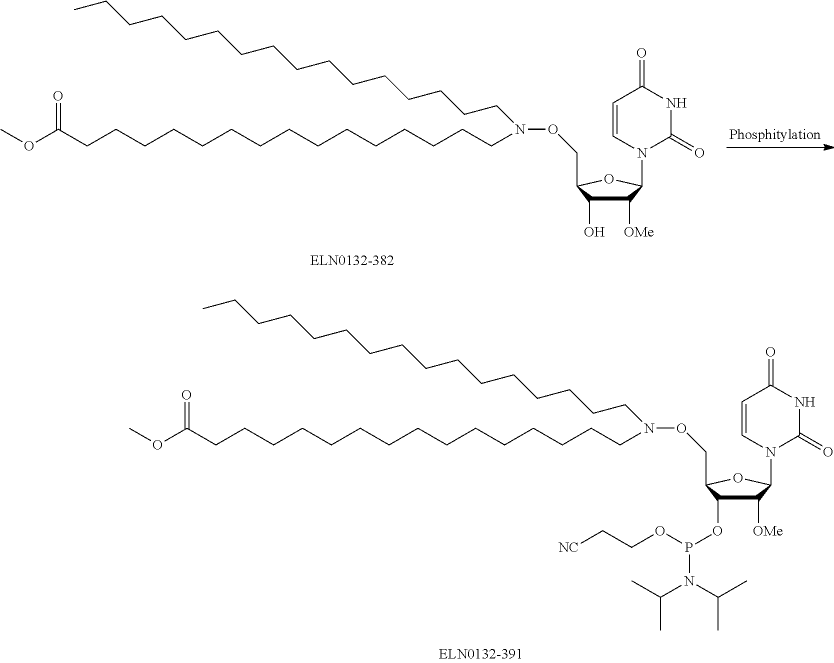

To a clear solution of ELN0132-382 in dichloromethane (30 mL) was added N-methylimidazole (208.97 mg, 2.55 mmol, 202.89 μL) and diisopropylethylamine (1.10 g, 8.48 mmol, 1.48 mL) in single portions. After stirring the reaction mixture for 5 minutes at 22° C., 2-cyanoethyl-N,N-diisopropylchlorophosphoramidite (803.25 mg, 3.39 mmol, 757.78 μL) was added and continued stirring for 1 hr and TLC was checked. Starting material was consumed and reaction mixture was diluted with DCM (15 mL). DCM layer was washed with 10% NaHCO₃ (2×25 mL) solution, and brine (30 mL). Organic layer was separated, dried over anhydrous Na₂SO₄, filtered and filtrate was evaporated at 36° C. to afforded crude compound which was purified by flash chromatography (60-100% EtOAc in hexane) to afford (1.31 g, 80% yield) as transparent gum. ¹H NMR (600 MHz, CD₃CN) δ 9.06 (s, 1H), 8.22-7.45 (m, 1H), 5.88 (dd, J=12.7, 4.4 Hz, 1H), 5.60 (dd, J=8.1, 1.4 Hz, 1H), 4.39-4.25 (m, 1H), 4.22-4.09 (m, 1H), 3.99 (td, J=10.7, 2.6 Hz, 1H), 3.91-3.75 (m, 4H), 3.76-3.60 (m, 2H), 3.60 (s, 3H), 3.50-3.36 (m, 3H), 2.72-2.58 (m, 6H), 2.27 (t, J=7.5 Hz, 2H), 1.61-1.46 (m, 7H), 1.38-1.25 (m, 49H), 1.19 (td, J=6.5, 3.1 Hz, 13H), 0.88 (t, J=7.0 Hz, 3H) ppm. ¹³C NMR (151 MHz, CD₃CN) δ 174.80, 163.86, 151.39, 141.00, 140.97, 119.51, 119.41, 102.55, 88.25, 87.89, 83.74, 83.72, 83.36, 83.34, 83.23, 83.20, 83.01, 82.97, 72.84, 72.73, 71.79, 71.70, 71.52, 71.41, 60.96, 59.76, 59.72, 59.70, 59.64, 59.30, 59.17, 59.01, 58.99, 58.57, 58.55, 55.32, 51.84, 44.13, 44.12, 44.05, 44.03, 34.52, 32.67, 30.43, 30.41, 30.40, 30.38, 30.34, 30.33, 30.31, 30.27, 30.25, 30.23, 30.11, 30.01, 29.80, 28.16, 28.14, 27.86, 25.71, 25.08, 25.03, 24.99, 24.95, 24.93, 24.90, 23.42, 21.01, 20.97, 14.52, 14.43 ppm. ³¹P NMR (243 MHz, CD₃CN) δ 150.01, 149.85 ppm.

### 1-[(2R,5R)-4-[tert-butyl(dimethyl)silyl]oxy-3-methoxy-5-[[methyl-[9-[2-[(2-pentylcyclopropyl)methyl]cyclopropyl]-]-[8-[2-[(2-pentylcyclopropyl)methyl]cyclopropyl]octyl] nonyl]amino]oxymethyl]tetrahydrofuran-2-yl]pyrimidine-2,4-dione ELN0132-390

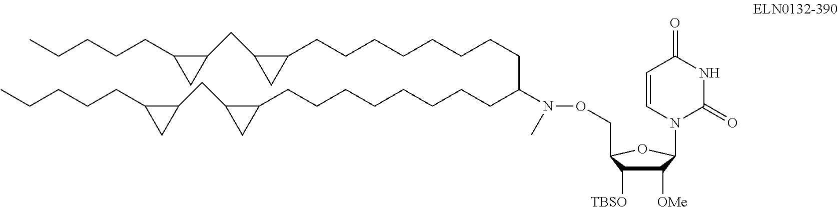

To a clear solution of 1-[(2R,5R)-4-[tert-butyl(dimethyl)silyl]oxy-3-methoxy-5-[[[9-[2-[(2-pentylcyclopropyl)methyl]cyclopropyl]-1-[8-[2-[(2-pentylcyclopropyl)methyl]cyclopropyl]octyl]nonylidene]amino]oxymethyl]tetrahydrofuran-2-yl]pyrimidine-2,4-dione (0.56 g, 587.92 μmol) in glacial acetic acid (10 mL) and DCM (3 mL) at 15° C. was added sodium cyanoborohydride (96.06 mg, 1.53 mmol) in single portion and stirred for 1 hr. To this resulting reaction mixture, formaldehyde, (190.84 mg, 2.35 mmol, 175.08 μL, 37% purity) was added slowly and stirred for 1 hr. To this mixture was added sodium cyanoborohydride (96.06 mg, 1.53 mmol) and stirred for 2 hrs at 15° C. Reaction mixture was diluted with DCM (20 mL) and washed with brine (30 mL). Organic layer separated, dried over anhydrous Na₂SO₄, filtered and filtrate was evaporated to dryness. The crude residue thus obtained, was purified by combiflash chromatography (gradient: 10-50% EtOAc in hexane) to afford ELN0132-390 (0.56 g, 98% yield) as transparent gum. ¹H NMR (600 MHz, CDCl₃) δ 8.69 (s, 1H), 7.98 (d, J=8.1 Hz, 1H), 5.93 (d, J=2.5 Hz, 1H), 5.68 (dd, J=8.2, 2.0 Hz, 1H), 4.17-4.07 (m, 2H), 4.04 (dd, J=11.1, 2.4 Hz, 1H), 3.81 (dd, J=11.1, 2.5 Hz, 1H), 3.59 (dd, J=4.8, 2.5 Hz, 1H), 3.52 (s, 3H), 2.57 (s, 4H), 1.56-1.45 (m, 2H), 1.46-1.23 (m, 50H), 1.21-1.07 (m, 3H), 1.03 (dt, J=14.1, 7.9 Hz, 1H), 0.91 (s, 10H), 0.88 (d, J=7.1 Hz, 3H), 0.78 (dtd, J=15.5, 7.8, 2.2 Hz, 4H), 0.69 (dqd, J=15.0, 7.0, 3.9 Hz, 4H), 0.61 (td, J=8.4, 4.2 Hz, 4H), 0.10 (d, J=7.0 Hz, 6H), −0.23-0.32 (m, 4H) ppm. 13C NMR (151 MHz, CDCl₃) δ 163.17, 150.14, 140.28, 101.90, 87.76, 84.16, 82.84, 69.93, 69.60, 66.69, 58.35, 40.17, 32.05, 30.37, 30.21, 30.17, 30.12, 30.04, 29.94, 29.89, 29.86, 29.83, 29.06, 29.03, 28.90, 28.86, 28.18, 28.03, 27.04, 26.88, 25.84, 22.87, 18.25, 16.20, 16.08, 16.06, 15.81, 15.79, 14.27, 11.17, 10.99, −4.40, −4.71 ppm. HRMS calc. for C₅₈H₁₀₆N₃O₆Si [M+H]⁺ 968.7851, found 968.7858.

1-[(2R,5R)-4-hydroxyl-3-methoxy-5-[[methyl-[9-[2-[(2-pentylcyclopropyl)methyl] cyclopropyl]-1-[8-[2-[(2-pentylcyclopropyl)methyl]cyclopropyl]octyl]nonyl]amino]oxymethyl] tetrahydrofuran-2-yl]pyrimidine-2,4-dione ELN0132-393:

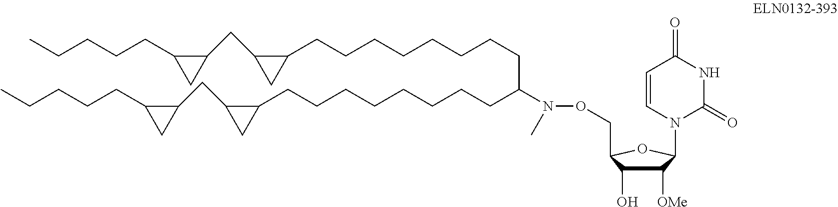

To a clear solution of ELN0132-390 (0.55 g, 567.86 mol) in THF (20 mL) at 22° C., tetrabutylammonium fluoride, 1M in THF (193.01 mg, 738.22 mol, 213.75 μL) was added slowly in single portion and then stirred for 3 hrs. All the volatile matters were removed under high vacuum pump and the residue thus obtained was purified by flash column chromatography (gradient: 20-50% EtOAc in hexane) to afford ELN0132-393 (0.4 g, 82% yield) as white semi-solid. ¹H NMR (600 MHz, CDCl₃) δ 8.50 (s, 1H), 7.96 (d, J=8.2 Hz, 1H), 5.96 (d, J=2.3 Hz, 1H), 5.69 (dd, J=8.2, 2.2 Hz, 1H), 4.19 (td, J=7.5, 5.2 Hz, 1H), 4.10 (dd, J=11.1, 2.3 Hz, 1H), 4.05 (dt, J=7.0, 2.4 Hz, 1H), 3.86 (dd, J=11.2, 2.5 Hz, 1H), 3.73 (dd, J=5.2, 2.3 Hz, 1H), 3.61 (s, 3H), 2.64 (d, J=8.1 Hz, 1H), 2.58 (s, 3H), 2.54 (q, J=5.4 Hz, 1H), 1.50 (tt, J=14.1, 5.9 Hz, 2H), 1.39 (ddq, J=9.1, 6.4, 3.4 Hz, 11H), 1.37-1.24 (m, 38H), 1.03 (dt, J=14.2, 8.0 Hz, 1H), 0.94-0.85 (m, 6H), 0.83-0.74 (m, 4H), 0.69 (ptd, J=8.6, 5.3, 3.0 Hz, 4H), 0.61 (td, J=8.4, 4.1 Hz, 4H), -0.28 (dq, J=17.1, 5.1 Hz, 4H) ppm. ¹³C NMR (151 MHz, CDCl₃) δ 162.92, 150.06, 140.07, 102.06, 87.21, 83.98, 83.13, 70.09, 68.69, 66.75, 58.80, 40.43, 32.04, 30.38, 30.17, 30.08, 30.04, 29.86, 29.83, 29.05, 29.02, 28.89, 28.86, 28.17, 28.01, 26.95, 26.85, 22.87, 16.18, 16.06, 16.04, 15.80, 15.78, 14.29, 11.16, 10.97 ppm. HRMS calc. for C₅₂H₉₂N₃O₆ [M+H]⁺ 854.6986, found 854.6960.

### [(2R,3R,4R,5R,6R)-5-acetamido-3,4-dibenzoyloxy-6-[5-[6-[[(2R,5R)-3-[tert-butyl(dimethyl)silyl]oxy-5-(2,4-dioxopyrimidin-1-yl)-4-methoxy-tetrahydrofuran-2-yl]methoxyimino]hexyl amino]-5-oxo-pentoxy]tetrahydropyran-2-yl]methylbenzoate ELN0132-22

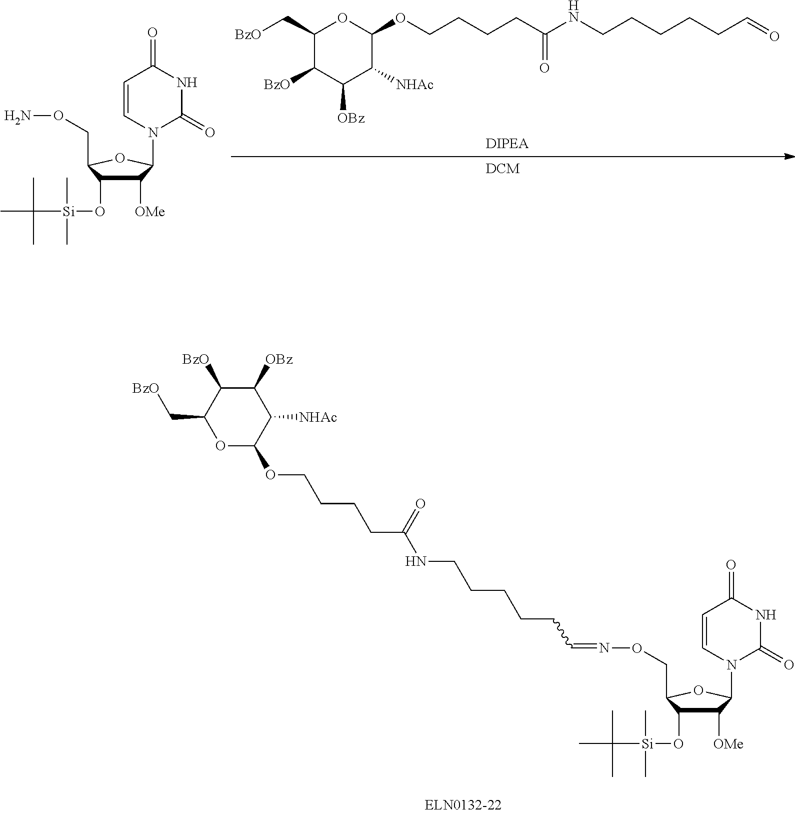

To a clear solution of 1-[(2R,5R)-5-(aminooxymethyl)-4-[tert-butyl(dimethyl)silyl]oxy-3-methoxy-tetrahydrofuran-2-yl]pyrimidine-2,4-dione (0.05 g, 0.13 mmol) in dry DCM (4 mL), DIPEA (50.53 mg, 0.39 mmol, 0.07 mL) was added at 25° C. The resulting mixture was stirred for 5 minutes and then added [(2R,3R,4R,5R,6R)-5-acetamido-3,4-dibenzoyloxy-6-[5-oxo-5-(6-oxohexylamino)pentoxy]tetra hydropyran-2-yl]methylbenzoate (94.30 mg, 0.13 mmol) in single portion. The reaction mixture was stirred for 16 h and TLC showed consumption of starting materials. All the volatile matters were evaporated to dryness and the gummy residue obtained, was purified by flash column chromatography (Solvent gradient: 0-7% methanol in DCM) to afford white solid compound ELN0132-22 (0.101 g, 71% yield). ¹H NMR (500 MHz, DMSO-d₆) δ 11.39 (d, J=5.7 Hz, 1H), 7.98-7.90 (m, 5H), 7.70 (dddd, J=10.3, 6.7, 3.5, 1.8 Hz, 5H), 7.67-7.63 (m, 1.5H), 7.58 (ddd, J=8.3, 7.4, 6.3 Hz, 3H), 7.53-7.45 (m, 3H), 7.43-7.34 (m, 2H), 6.81 (t, J=5.4 Hz, 0.5H), 5.80 (dd, J=5.9, 4.5 Hz, 1H), 5.75 (d, J=3.4 Hz, 1H), 5.66 (ddd, J=26.6, 8.2, 1.7 Hz, 1H), 5.37 (dd, J=11.1, 3.4 Hz, 1H), 4.73 (d, J=8.5 Hz, 1H), 4.49-4.41 (m, 2H), 4.38-4.32 (m, 1H), 4.30-4.23 (m, 2.5H), 4.19 (dd, J=12.2, 4.3 Hz, 0.5H), 4.11 (ddd, J=20.7, 12.2, 4.5 Hz, 1H), 4.00 (dq, J=11.5, 4.5 Hz, 1H), 3.89 (td, J=4.7, 1.5 Hz, 1H), 3.80 (dd, J=10.6, 5.2 Hz, 1H), 3.51 (dt, J=9.4, 5.7 Hz, 1H), 3.34 (d, J=6.4 Hz, 3H), 3.01 (q, J=6.5 Hz, 2H), 2.24 (dt, J=8.1, 6.6 Hz, 1H), 2.12 (q, J=7.0 Hz, 1H), 2.09-1.97 (m, 2H), 1.70 (s, 3H), 1.51 (d, J=3.5 Hz, 4H), 1.40 (dq, J=21.8, 7.4 Hz, 4H), 1.27 (q, J=7.9 Hz, 2H), 0.86 (s, 9H), 0.07 (dd, J=3.1, 1.3 Hz, 6H) ppm. ¹³C NMR (126 MHz, DMSO-d₆) δ 171.72, 169.36, 165.20, 165.15, 164.86, 163.00, 152.49, 151.93, 150.41, 150.36, 140.27, 140.10, 133.78, 133.51, 133.48, 129.21, 129.17, 129.03, 128.98, 128.71, 128.59, 102.03, 101.88, 100.89, 86.84, 86.67, 82.32, 82.13, 81.44, 81.28, 79.16, 72.04, 71.84, 69.97, 69.86, 69.70, 68.75, 67.92, 62.03, 57.65, 57.57, 49.74, 38.18, 38.16, 35.03, 28.89, 28.75, 28.59, 26.16, 25.93, 25.62, 25.58, 25.36, 25.20, 22.70, 21.86, −4.85, −4.88, −5.06, −5.15 ppm. HRMS calc. for C₅₆H₇₄N₄O₁₆Si [M+H]⁺ 1122.4719, found 1122.4738.

### [(3S,10R,13R,17R)-17-[(1R)-1,5-dimethylhexyl]-10,13-dimethyl-2,3,4,7,8,9,11,12,14,15,16,17-dodecahydro-1H-cyclopenta[a]phenanthren-3-yl]-N-[(6Z)-6-[[(2R,5R)-3-[tert-butyl(dimethyl) silyl]oxy-5-(2,4-dioxopyrimidin-1-yl)-4-methoxy-tetrahydrofuran-2-yl]methoxyimino]hexyl]carbamate ELN0132-12

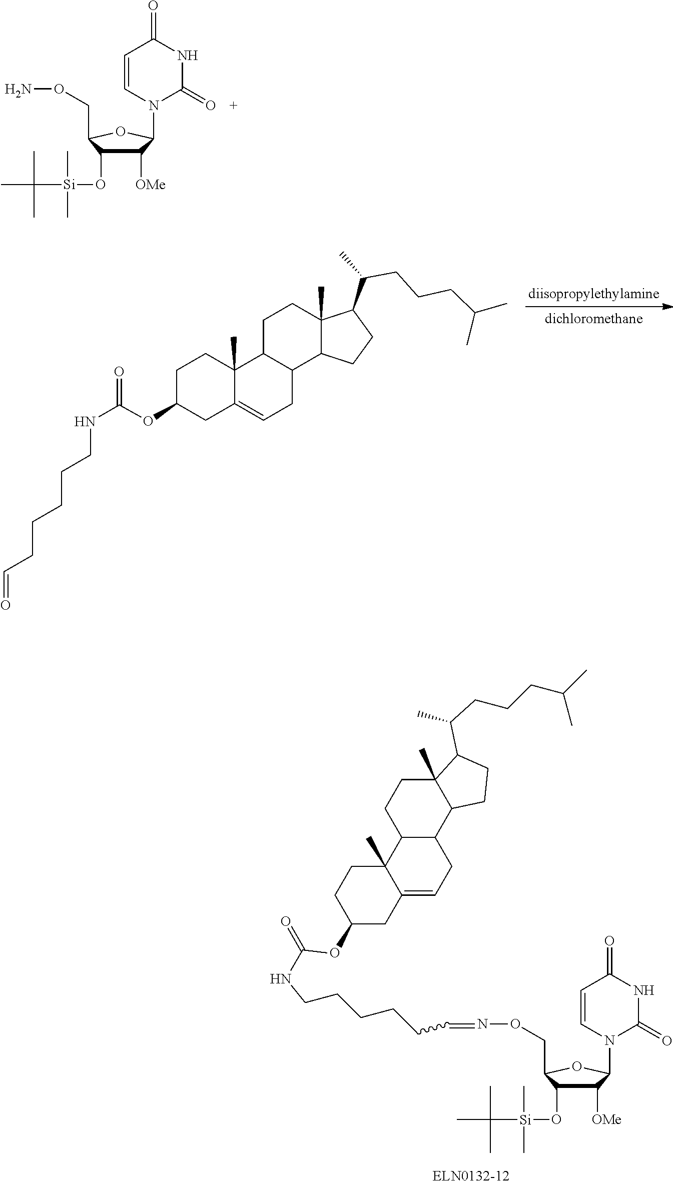

To a solution of 1-[(2R,5R)-5-(aminooxymethyl)-4-[tert-butyl(dimethyl)silyl]oxy-3-methoxy-tetrahydrofuran-2-yl]pyrimidine-2,4-dione (0.24 g, 619.35 umol) in DCM (4 mL), DIPEA (242.56 mg, 1.86 mmol, 326.90 μL) was added at 25° C. To this reaction mixture, [(3S,10R,13R,17R)-17-[(1R)-1,5-dimethylhexyl]-10,13-dimethyl-2,3,4,7,8,9,11,12,14,15,16,17-dodecahydro-1H-cyclopenta[a]phenanthren-3-yl]-N-(6-oxohexyl) carbamate (526.91 mg, 998.28 umol) was added in single portion and resulting clear solution was stirred for 20 h. The reaction mixture was diluted with DCM (20 mL), washed with water (10 mL), brine (2×20 mL) and organic layer was separated. DCM layer was dried over anhydrous Na₂SO₄, filtered and the filtrate was evaporated to dryness. The crude compound was purified by combiflash chromatography (Gradient: 10-60% EA in hexane) to afford ELN0132-12 (0.527 g, 95% yield). ¹H NMR (400 MHz, CDCl₃, TMS, 25° C.) δ 9.19 (s, 1H), 7.75 (dd, J=19.0, 8.1 Hz, 1H), 7.39 (t, J=6.1 Hz, 0.6H), 6.69 (t, J=5.3 Hz, 0.4H), 5.98-5.78 (m, 1H), 5.66 (ddd, J=13.3, 8.1, 2.2 Hz, 1H), 5.37 (dd, J=4.5, 2.0 Hz, 1H), 4.70 (d, J=25.7 Hz, 1H), 4.54-4.36 (m, 2H), 4.29-4.14 (m, 3H), 3.66 (dt, J=4.6, 2.5 Hz, 1H), 3.55 (d, J=4.8 Hz, 3H), 3.16 (q, J=6.6 Hz, 2H), 2.51-2.16 (m, 4H), 2.07-1.72 (m, 6H), 1.60-1.29 (m, 17H), 1.28-1.04 (m, 10H), 1.02-0.95 (m, 5H), 0.91 (s, 13H), 0.86 (dd, J=6.6, 1.8 Hz, 6H), 0.67 (s, 3H), 0.14-0.04 (m, 6H) ppm. ¹³C NMR (101 MHz, CDCl₃, 25° C.) δ 163.42, 156.31, 152.35, 151.57, 150.16, 150.12, 140.13, 139.98, 122.59, 101.94, 88.62, 83.79, 82.71, 82.66, 74.40, 71.69, 71.32, 69.60, 69.44, 58.64, 58.59, 56.83, 56.28, 50.16, 42.45, 40.79, 39.88, 39.66, 38.72, 37.14, 36.70, 36.33, 35.93, 32.05, 32.02, 29.89, 29.83, 29.43, 28.36, 28.33, 28.14, 26.63, 26.36, 26.00, 25.83, 24.42, 23.97, 22.95, 22.70, 21.18, 19.47, 18.86, 18.27, 12.00, -4.55, −4.58, −4.72, −4.79 ppm. HRMS calc. for C₅₀H₈₅N₄O₈Si [M+H]⁺ 897.6137, found 897.6114.

### Methyl-(2S)-5-[[3-[tert-butyl(dimethyl)silyl]oxy-5-(2,4-dioxopyrimidin-1-yl)-4-methoxy-tetrahydrofuran-2-yl]methoxy-hexadecyl-amino]-2-[[4-[[2-(2-methylpropanoylamino)-4-oxo-3H-pteridin-6-yl]methyl-(2,2,2-trifluoroacetyl)amino]benzoyl]amino]-5-oxo-pentanoate ELN0132-392

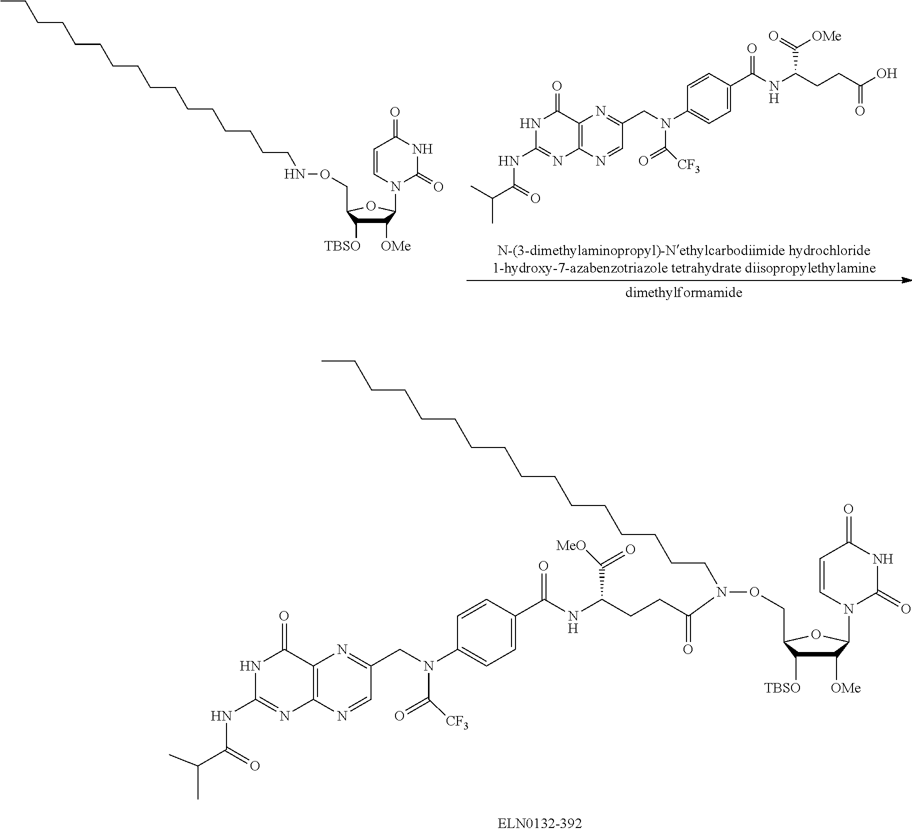

To a clear solution of (4S)-5-methoxy-4-[[4-[[2-(2-methylpropanoylamino)-4-oxo-3H-pteridin-6-yl]methyl-(2,2,2-trifluoroacetyl)amino]benzoyl]amino]-5-oxo-pentanoic acid (0.56 g, 901.02 mol) in dimethylformamide (4 mL), were added N-(3-dimethylaminopropyl)-N′-ethylcarbodiimide hydrochloride (172.72 mg, 901.02 mol) 1-hydroxyl-7-azabenzotriazole tetrahydrate (187.56 mg, 901.02 mol) and diisopropylethylamine (349.34 mg, 2.70 mmol, 470.81 μL) in single portions. After 5 minutes, 1-[4-[tert-butyl(dimethyl)silyl]oxy-5-[(hexadecylamino)oxymethyl]-3-methoxy-tetrahydrofuran-2-yl]pyrimidine-2,4-dione (551.36 mg, 901.02 mol) was added and the resulting mixture was stirred for 10 hr at 25° C. All the volatile matters were removed under high vacuum pump and the residue was diluted with DCM (30 mL), and water (20 mL). Organic layer was separated, washed with NaHCO₃ solution (20 mL), water (20 mL) and brine (30×3 mL). DCM layer was separated, dried on anhydrous Na₂SO₄, filtered and the filtrate was evaporated to dryness. The crude mass obtained, was purified by combiflash chromatography (gradient: 0-5% MeOH in DCM) to afford ELN0132-392 (0.81 g, 666.43 mol, 73.96% yield) as white solid. ¹H NMR (600 MHz, CDCl₃) δ 12.54 (s, 1H), 10.14 (s, 1H), 9.99 (s, 1H), 8.96 (s, 1H), 7.85-7.80 (m, 2H), 7.63-7.47 (m, 2H), 7.42 (d, J=8.2 Hz, 2H), 5.76 (s, 1H), 5.64 (dd, J=8.3, 2.1 Hz, 1H), 5.29 (d, J=11.8 Hz, 1H), 5.21 (d, J=15.4 Hz, 1H), 4.74-4.67 (m, 1H), 4.20-4.13 (m, 2H), 4.08 (d, J=11.5 Hz, 2H), 3.75 (s, 3H), 3.69 (s, 2H), 3.52 (s, 3H), 2.82 (s, 1H), 2.68 (s, 2H), 2.34 (d, J=14.6 Hz, 1H), 2.21-2.12 (m, 1H), 1.59 (s, 2H), 1.30-1.22 (m, 35H), 0.90 (s, 10H), 0.87 (t, J=7.0 Hz, 3H), 0.09 (d, J=11.2 Hz, 6H) ppm. ¹³C NMR (151 MHz, CDCl₃) δ 180.53, 173.67, 173.54, 172.73, 171.44, 169.17, 165.93, 163.42, 159.63, 157.76, 157.52, 157.28, 157.03, 154.82, 151.30, 150.04, 149.88, 149.00, 148.83, 142.27, 141.95, 139.83, 134.48, 131.06, 130.71, 130.63, 128.98, 128.61, 128.18, 119.06, 117.15, 115.24, 113.33, 102.54, 89.41, 83.00, 81.02, 72.36, 69.70, 58.63, 58.38, 54.56, 53.58, 53.03, 52.99, 52.79, 46.06, 36.50, 32.05, 29.84, 29.82, 29.79, 29.78, 29.72, 29.68, 29.50, 29.47, 27.29, 26.90, 25.75, 22.83, 19.05, 19.02, 19.00, 18.98, 18.20, 14.27, 0.13, −4.36, −4.86 ppm. ¹⁹F NMR (565 MHz, CDCl₃) δ −67.03 ppm. HRMS calc. for C₅₈H₈₆F₃N₁₀O₁₃Si [M+H]⁺ 1215.6097, found 1215.6097.

1-[(2R,5R)-4-hydroxyl-3-methoxy-5-[[[(10Z,13Z)-1-[(9Z,12Z)-octadeca-9,12-dienyl]nonadeca-10,13-dienylidene]amino]oxymethyl]tetrahydrofuran-2-yl]pyrimidine-2,4-dione ELN0132-66:

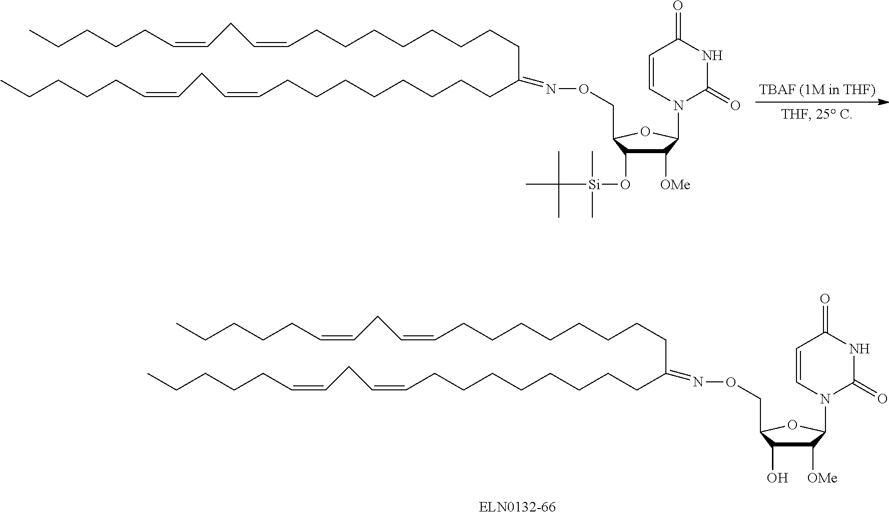

To a clear solution of 1-[(2R,5R)-4-[tert-butyl(dimethyl)silyl]oxy-3-methoxy-5-[[[(10Z,13Z)-1-[(9Z,12Z)-octadeca-9,12-dienyl]nonadeca-10,13-dienylidene]amino]oxymethyl]tetrahydrofuran-2-yl]pyrimidine-2,4-dione (0.7 g, 780.90 umol) in tetrahydrofuran (20 mL), was added tetrabutylammonium fluoride (312.51 mg, 1.17 mmol, 1M in THF) at 22° C. The reaction mixture was stirred for 12 h and then volatile matters were evaporated under high vacuum pump. The crude residue thus obtained, was purified by combiflash chromatography (Gradient: 10-50% EA in hexane) to afford ELN0132-66 (0.37 g, 61% yield). ¹H NMR (400 MHz, CDCl₃, TMS, 25° C.) δ 10.08 (d, J=2.1 Hz, 1H), 7.71 (d, J=8.1 Hz, 1H), 5.94 (d, J=2.3 Hz, 1H), 5.65 (dd, J=8.1, 1.9 Hz, 1H), 5.44-5.07 (m, 8H), 4.41 (dd, J=12.6, 2.4 Hz, 1H), 4.29-4.17 (m, 2H), 4.11 (dt, J=7.2, 2.7 Hz, 1H), 3.73 (dd, J=5.1, 2.4 Hz, 1H), 3.59 (s, 3H), 2.92 (d, J=7.8 Hz, 1H), 2.75 (t, J=6.4 Hz, 4H), 2.31-2.20 (m, 2H), 2.18-2.08 (m, 2H), 2.08-1.90 (m, 8H), 1.45 (q, J=7.8 Hz, 4H), 1.39-1.13 (m, 34H), 0.86 (t, J=6.8 Hz, 6H).ppm. ¹³C NMR (101 MHz, CDCl₃, 25° C.) δ 163.72, 162.72, 150.39, 139.62, 130.21, 130.11, 130.09, 128.07, 128.05, 127.98, 102.19, 87.47, 83.89, 83.36, 71.67, 68.95, 58.74, 34.02, 31.58, 29.98, 29.70, 29.51, 29.48, 29.43, 29.40, 29.32, 28.32, 27.28, 27.26, 26.61, 25.93, 25.70, 22.62, 14.12 ppm. HRMS calc. for C₄₇H₈₀N₃O₆[M+H]⁺ 782.6047, found 782.6056.

### 3-[(diisopropylamino)-[(2R,5R)-5-(2,4-dioxopyrimidin-1-yl)-4-methoxy-2-[[[(10Z,13Z)-1-[(9Z,12Z)-octadeca-9,12-dienyl]nonadeca-10,13-dienylidene]amino]oxymethyl]tetrahydrofuran-3-yl]oxy-phosphanyl]oxypropanenitrile ELN0132-69

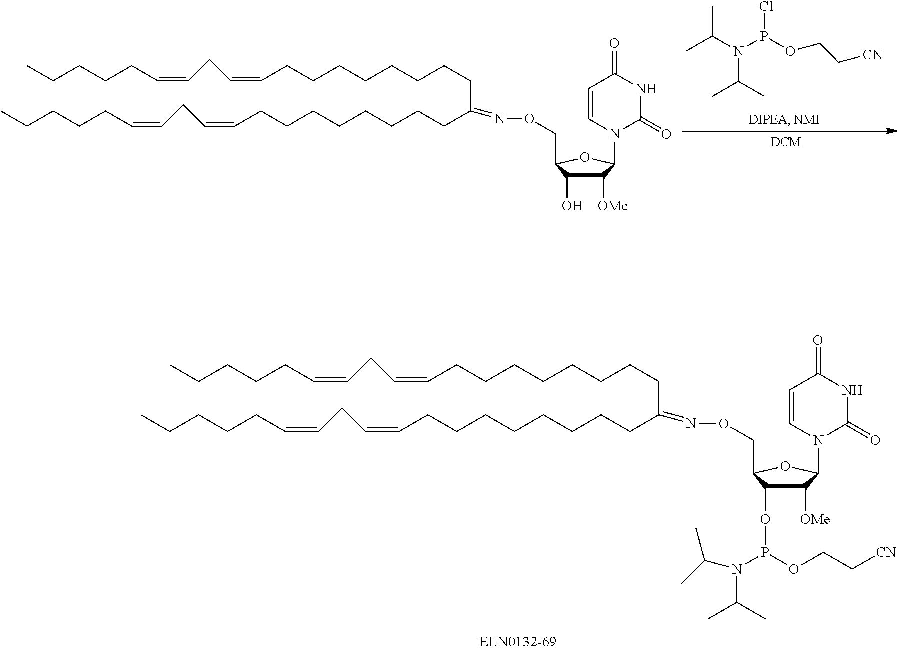

To a clear solution of ELN0132-66 (0.3 g, 0.38 mmol) in DCM (10 mL), was added DIPEA (0.248 g, 1.92 mmol, 0.33 mL) and NMI (0.11 g, 1.34 mmol, 0.11 mL) at 22° C. The reaction mixture was stirred for 5 minutes and 2-cyanoethyl-N,N-diisopropylchlorophosphoramidite (0.182 g, 0.77 mmol, 0.17 mL) was added in single portion. Stirring was continued for 1 h at 22° C. after which the reaction mixture was diluted with DCM (20 mL) and saturated NaHCO₃ solution (20 mL) was added. Organic layer was separated and washed with brine (30 mL). DCM layer was separated, dried over anhydrous Na₂SO₄, filtered, and the filtrate was evaporated to dryness. The crude mass thus obtained, was purified by combiflash chromatography to afford ELN0132-69 (0.3 g, 80% yield) as transparent gum. ¹H NMR (500 MHz, CDCl₃, TMS, 25° C.) δ 8.34 (s, 1H), 7.70 (dd, J=13.3, 8.2 Hz, 1H), 6.01 (t, J=4.0 Hz, 1H), 5.65 (dd, J=8.1, 4.5 Hz, 1H), 5.44-5.15 (m, 8H), 4.50-4.34 (m, 2.5H), 4.30-4.16 (m, 1.5H), 3.96-3.83 (m, 1.7H), 3.81-3.70 (m, 1.3H), 3.65 (dddd, J=14.8, 10.5, 7.3, 6.0 Hz, 2H), 3.52 (s, 1.8H), 3.47 (s, 1.2H), 2.77 (t, J=6.8 Hz, 4H), 2.70-2.56 (m, 2H), 2.27 (ddd, J=12.8, 6.5, 4.3 Hz, 2H), 2.16 (dt, J=8.3, 6.1 Hz, 2H), 2.04 (dd, J=7.8, 6.3 Hz, 8H), 1.47 (q, J=8.1 Hz, 4H), 1.41-1.24 (m, 35H), 1.26-1.09 (m, 13H), 0.89 (t, J=6.9 Hz, 6H).ppm. ¹³C NMR (101 MHz, CDCl₃, 25° C.) δ ppm. ³¹P NMR (202 MHz, CDCl₃, 25° C.) δ 152.62, 151.84 ppm. HRMS calc. for C₅₆H₉₇N₅O₇P [M+H]⁺ 1004.6945, found 1004.6978.

### [(2R,3R,4R,5R,6R)-5-acetamido-6-[5-[3-[3-[3-[3-[3-[5-[(2R,3R,4R,5R,6R)-3-acetamido-4,5-dibenzoyloxy-6-(benzoyloxymethyl)tetrahydropyran-2-yl]oxypentanoylamino]propylamino]-3-oxo-propoxy]-2-[[3-[3-[5-[(2R,3R,4R,5R,6R)-3-acetamido-4,5-dibenzoyloxy-6-(benzoyloxymethyl)tetrahydropyran-2-yl]oxypentanoylamino]propylamino]-3-oxo-propoxy]methyl]-2-[[12-[[(6E)-6-[[(2R,5R)-3-[tert-butyl(dimethyl)silyl]oxy-5-(2,4-dioxopyrimidin-1-yl)-4-methoxy-tetrahydrofuran-2-yl]methoxyimino]hexyl]amino]-12-oxo-dodecanoyl]amino]propoxy]propanoylamino]propylamino]-5-oxo-pentoxy]-3,4-dibenzoyloxy-tetrahydropyran-2-yl]methyl benzoate ELN0132-49

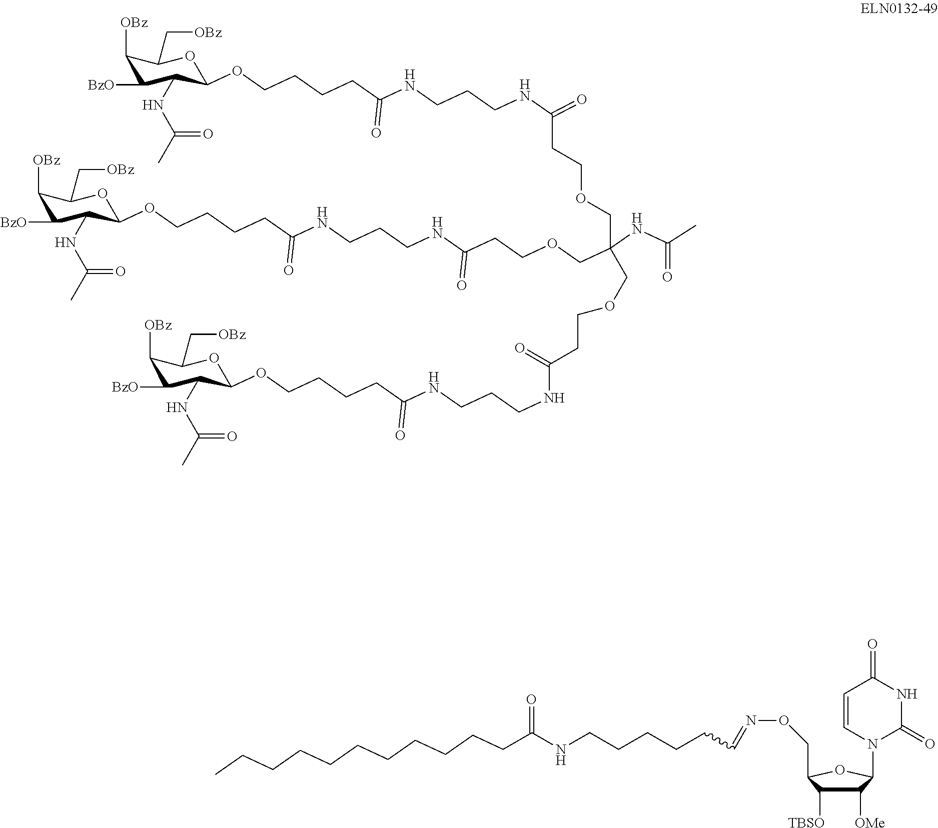

To clear solution of ELN0132-6 (0.125 g, 322.58 umol) in dry dichloromethane (20 mL), diisopropylethylamine (126.33 mg, 967.74 umol, 170.26 uL) was added at 25° C. The resulting mixture was stirred for 5 minutes and then added TriGalNAc-aldehyde (858.69 mg, 322.58 umol) in a single portion. The reaction mixture was stirred for 18 h and TLC was checked which showed consumption of starting materials. All the volatile matters were evaporated to dryness and the gummy residue obtained, was purified by flash column chromatography (gradient: 0-10% methanol in dichloromethane) to afford ELN0132-49 as white solid (0.91 g, 93% yield). ¹H NMR (400 MHz, CDCl₃) δ 8.11-8.04 (m, 5H), 8.03-7.96 (m, 5H), 7.81 (dt, J=7.9, 1.1 Hz, 5H), 7.61-7.37 (m, 19H), 7.33-7.23 (m, 12H), 7.02 (d, J=8.7 Hz, 5H), 6.70-6.45 (m, 1H), 5.98-5.79 (m, 4H), 5.67-5.57 (m, 2H), 4.81 (d, J=8.3 Hz, 2H), 4.64 (dd, J=11.2, 6.5 Hz, 3H), 4.39 (ddd, J=17.8, 11.2, 7.8 Hz, 5H), 4.30-4.12 (m, 4H), 4.02 (dd, J=10.3, 5.4 Hz, 2H), 3.69-3.43 (m, 18H), 3.27 (d, J=7.0 Hz, 13H), 2.39 (s, 1H), 2.35-2.06 (m, 6H), 1.86 (d, J=4.0 Hz, 10H), 1.82-1.43 (m, 19H), 1.21 (s, 9H), 0.90 (d, J=1.2 Hz, 9H), 0.08 (dd, J=3.6, 2.7 Hz, 6H) ppm.

[(2R,3R,4R,5R,6R)-5-acetamido-6-[5-[3-[3-[3-[3-[3-[5-[(2R,3R,4R,5R,6R)-3-acetamido-4,5-dibenzoyloxy-6-(benzoyloxymethyl)tetrahydropyran-2-yl]oxypentanoylamino]propylamino]-3-oxo-propoxy]-2-[[3-[3-[5-[(2R,3R,4R,5R,6R)-3-acetamido-4,5-dibenzoyloxy-6-(benzoyloxymethyl)tetrahydropyran-2-yl]oxypentanoylamino]propylamino]-3-oxo-propoxy]methyl]-2-[[12-[[(6E)-6-[[(2R,5R)-5-(2,4-dioxopyrimidin-1-yl)-3-hydroxyl-4-methoxy-tetrahydrofuran-2-yl]methoxyimino]hexyl]amino]-12-oxo-dodecanoyl]amino]propoxy]propanoylamino]propylamino]-5-oxo-pentoxy]-3,4-dibenzoyloxy-tetrahydropyran-2-yl]methyl benzoate ELN0132-110:

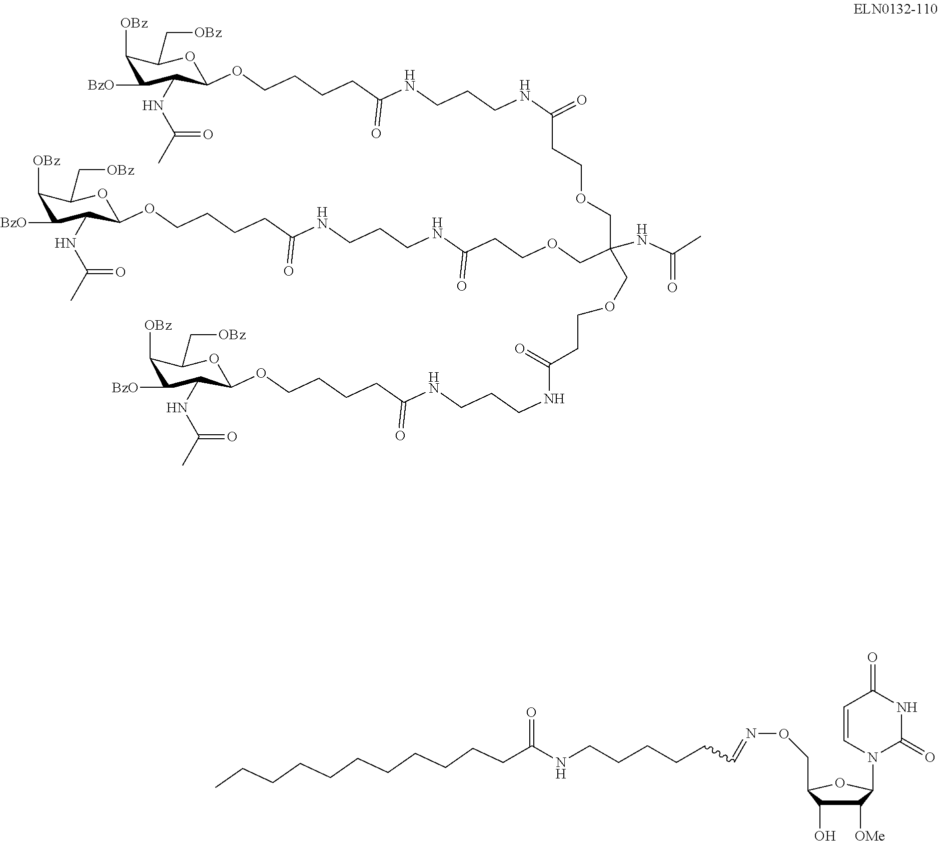

To a solution of ELN0132-49 (0.25 g, 82.47 μmol) in THF (15 mL) at 25° C., tetrabutylammonium fluoride (43.56 mg, 164.94 mol) was added slowly in single portion and then stirred for 24 hr. Volatile matters were removed in high vacuum pump and crude residue thus obtained was purified by combiflash chromatography (gradient: 1-10% MeOH: DCM) to afford ELN0132-110 (0.205 g, 85% yield). ¹H NMR (500 MHz, CDCl₃) δ 8.11-8.05 (m, 6H), 8.02-7.97 (m, 6H), 7.82 (dd, J=8.2, 1.4 Hz, 5H), 7.78-7.69 (m, 1H), 7.69-7.51 (m, 6H), 7.49-7.35 (m, 10H), 7.31-7.24 (m, 11H), 7.01 (d, J=10.5 Hz, 5H), 6.72-6.49 (m, 1H), 5.91 (dd, J=16.5, 3.1 Hz, 3H), 5.69-5.58 (m, 2H), 4.82 (d, J=8.3 Hz, 2H), 4.64 (dd, J=11.2, 6.5 Hz, 3H), 4.48-4.38 (m, 4H), 4.34-4.21 (m, 4H), 4.15 (t, J=8.1 Hz, 1H), 4.01 (dt, J=10.1, 5.2 Hz, 2H), 3.76 (d, J=5.1 Hz, 1H), 3.66-3.52 (m, 12H), 3.33-3.12 (m, 14H), 2.40 (d, J=5.0 Hz, 5H), 2.32-2.05 (m, 7H), 1.87 (s, 8H), 1.80-1.42 (m, 8H), 1.35-1.12 (m, 11H) ppm. Obsd. MALDI MS: 2941.67 [calcd. Molecular Weight for Chemical Formula: C152H190N14NaO44, 2940.2358].

### [(2R,3R,4R,5R,6R)-5-acetamido-6-[5-[3-[3-[3-[3-[3-[5-[(2R,3R,4R,5R,6R)-3-acetamido-4,5-dibenzoyloxy-6-(benzoyloxymethyl)tetrahydropyran-2-yl]oxypentanoylamino]propylamino]-3-oxo-propoxy]-2-[[3-[3-[5-[(2R,3R,4R,5R,6R)-3-acetamido-4,5-dibenzoyloxy-6-(benzoyloxymethyl)tetrahydropyran-2-yl]oxypentanoylamino]propylamino]-3-oxo-propoxy]methyl]-2-[[12-[[(6E)-6-[[(2R,5R)-3-[2-cyanoethoxy-(diisopropylamino)phosphanyl]oxy-5-(2,4-dioxopyrimidin-1-yl)-4-methoxy-tetrahydrofuran-2-yl]methoxyimino]hexyl]amino]-12-oxo-dodecanoyl]amino]propoxy]propanoylamino]propylamino-5-oxo-pentoxy]-3,4-dibenzoyloxy-tetrahydropyran-2-yl]methyl benzoate ELN0132-521

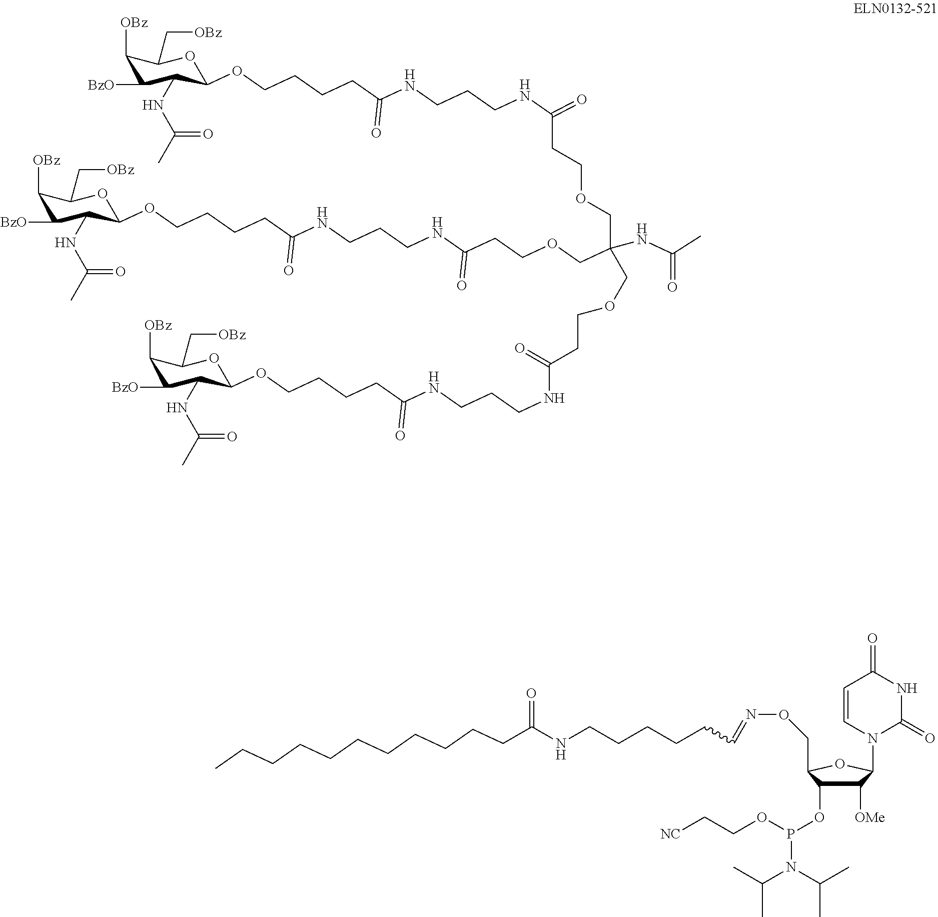

To a clear solution of ELN0132-110 (0.3 g, 102.84 mol) in dichloromethane (5 mL) was added N-methylimidazole (12.66 mg, 154.26 mol, 12.30 μL) and diisopropylethylamine (66.45 mg, 514.19 mol, 89.56 μL) in single portions. After stirring the reaction mixture for 5 minutes at 22° C., 2-cyanoethyl-N,N-diisopropylchlorophosphoramidite (48.68 mg, 205.68 mol, 45.92 μL) was added and continued stirring for 1 hr and TLC was checked. Starting material was consumed and reaction mixture was diluted with DCM (15 mL). DCM layer was washed with 10% NaHCO₃ (2×25 mL) solution, and brine (30 mL). Organic layer was separated, dried over anhydrous Na₂SO₄, filtered and filtrate was evaporated at 36° C. to afford crude compound which was purified by flash chromatography (0-3% MeOH in DCM with 3% TEA) to afford ELN0132-521 (0.27 g, 84% yield) as white foam. ³¹P NMR (243 MHz, CD₃CN) δ 150.12, 150.07, 149.90 ppm.

### LNA-A Analogues:

### Methyl-16-[[(4R,6R)-6-(6-aminopurin-9-yl)-7-[tert-butyl(dimethyl)silyl]oxy-2,5-dioxabicyclo[2.2.1]heptan-4-yl]methoxy-hexyl-amino]hexadecanoate ELN0132-272

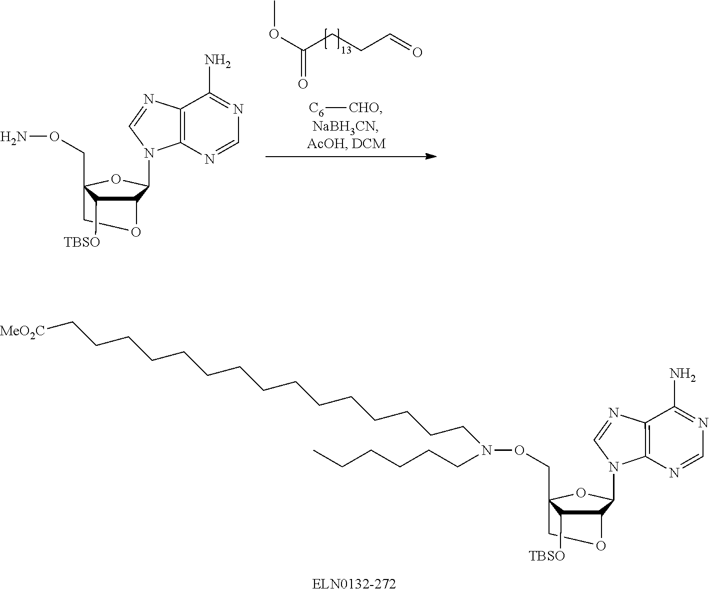

To a solution of 0-[[(4R,6R)-6-(6-aminopurin-9-yl)-7-[tert-butyl(dimethyl)silyl]oxy-2,5-dioxabicyclo[2.2.1]heptan-4-yl]methyl]hydroxylamine (0.7 g, 1.71 mmol) in DCM (13.87 mL) was added Hexanal (262.69 mg, 2.57 mmol, 314.97 μL, 98% purity) at 22° C. The resulting clear solution was stirred for 10 hr. The reaction mixture was diluted with DCM (20 mL), washed with water (10 mL), brine (2×20 mL) and organic layer was separated. DCM layer was dried over anhydrous Na₂SO₄, filtered and the filtrate was evaporated to dryness. The crude compound was purified by column chromatography (gradient: 10-60% EA in hexane) to afford the intermediate. This intermediate was again dissolved in a mixture of DCM (13.87 mL) and acetic acid (4.62 mL) and sodium cyanoborohydride (294.69 mg, 4.46 mmol, 95% purity) was added in portions. Reaction mixture was stirred for 1 hr and methyl 16-oxohexadecanoate (731.05 mg, 2.57 mmol, 501.94 μL) was added into it. Stirring continued for 1 hr and then second batch of sodium cyanoborohydride (294.69 mg, 4.46 mmol, 95% purity) was added. Reaction mixture was diluted with DCM (20 mL) after 3 hrs. Organic layer was washed with water (10 mL), brine (2×20 mL) and organic layer was separated. DCM layer was dried over anhydrous Na₂SO₄, filtered and the filtrate was evaporated to dryness. The crude compound was purified by column chromatography (gradient: 10-50% EA in hexane) to afford ELN0132-272 (0.60 g, 46% yield) as white solid. HRMS calc. for C₄₀H₇₃N₆O₆Si [M+H]⁺ 761.5361, found 761.5358.

### Methyl-16-[[(4R,6R)-7-[tert-butyl(dimethyl)silyl]oxy-6-[6-[(Z)-dimethylaminomethyleneamino]purin-9-yl]-2,5-dioxabicyclo[2.2.1]heptan-4-yl]methoxy-hexyl-amino]hexadecanoate ELN0132-381

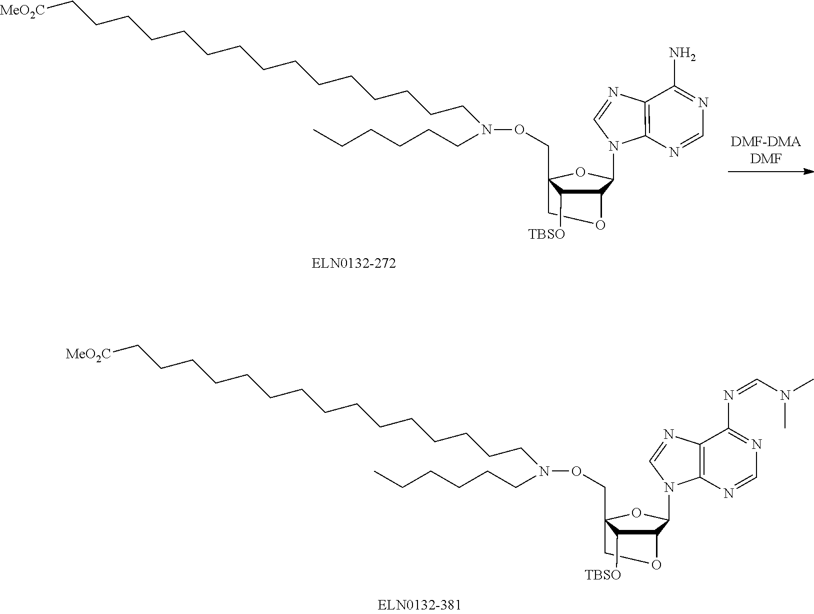 [SMILES: CCCCCCN(CCCCCCCCCCCCCCCC(=O)OC)OC[C@]12CO[C@@H](C1O[Si](C)(C)C(C)(C)C)[C@H](n1cnc3c(N)ncnc31)O2 ; CCCCCCN(CCCCCCCCCCCCCCCC(=O)OC)OC[C@]12CO[C@@H](C1O[Si](C)(C)C(C)(C)C)[C@H](n1cnc3c(/N=C\N(C)C)ncnc31)O2]

To a clear solution of methyl 16-[[(4R,6R)-6-(6-aminopurin-9-yl)-7-[tert-butyl(dimethyl)silyl]oxy-2,5-dioxabicyclo[2.2.1]heptan-4-yl]methoxy-hexyl-amino]hexadecanoate (0.4 g, 525.54 mol) in dimethylformamide was added N,N-dimethylformamide dimethyl acetal (93.94 mg, 788.31 mol, 105.55 μL) in single portion and the reaction mixture was stirred at 65° C. for 4 hrs. TLC was checked, and volatile matters was removed under high vacuum pump. Residue was dissolved in DCM (100 mL) and the organic layer was washed with brine (3×50 mL). DCM layer was then dried over anhydrous Na₂SO₄, filtered and the filtrate was evaporated to dryness. Crude mass thus obtained, was purified by flash column chromatography (gradient: 30-80% EtOAc in hexane) to afford ELN0132-381 (0.38 g, 89% yield) as transparent gum. ¹H NMR (600 MHz, CDCl₃) δ 8.95 (d, J=2.1 Hz, 1H), 8.52 (d, J=2.0 Hz, 1H), 8.13 (d, J=2.0 Hz, 1H), 6.00 (d, J=2.0 Hz, 1H), 4.66 (d, J=2.1 Hz, 1H), 4.31 (d, J=2.0 Hz, 1H), 4.10-4.05 (m, 2H), 4.02 (dd, J=10.9, 2.0 Hz, 1H), 3.95 (dd, J=7.7, 2.1 Hz, 1H), 3.66 (d, J=2.0 Hz, 3H), 3.27 (d, J=2.0 Hz, 3H), 3.21 (d, J=2.1 Hz, 3H), 2.67 (q, J=6.5 Hz, 4H), 2.29 (td, J=7.6, 2.1 Hz, 2H), 1.61 (t, J=7.1 Hz, 2H), 1.54 (q, J=7.5 Hz, 4H), 1.34-1.22 (m, 29H), 0.90-0.84 (m, 12H), 0.05-0.08 (m, 6H) ppm. ¹³C NMR (151 MHz, CDCl₃) δ 174.49, 159.72, 158.09, 152.85, 150.52, 139.80, 126.63, 86.89, 86.76, 79.17, 72.70, 72.14, 69.20, 59.51, 51.57, 41.42, 35.30, 34.26, 31.91, 29.81, 29.78, 29.76, 29.74, 29.72, 29.60, 29.40, 29.30, 27.63, 27.35, 27.29, 25.71, 25.10, 22.76, 17.99, 14.19, −4.60, −5.03 ppm. HRMS calc. for C₄₃H₇₈N₇O₆Si [M+H]⁺ 816.5783, found 816.5756.

### 9-[(4R,6R)-7-[tert-butyl(dimethyl)silyl]oxy-4-[(dihexadecylamino)oxymethyl]-2,5-dioxabicyclo[2.21]heptan-6-yl]purin-6-amine ELN0132-168

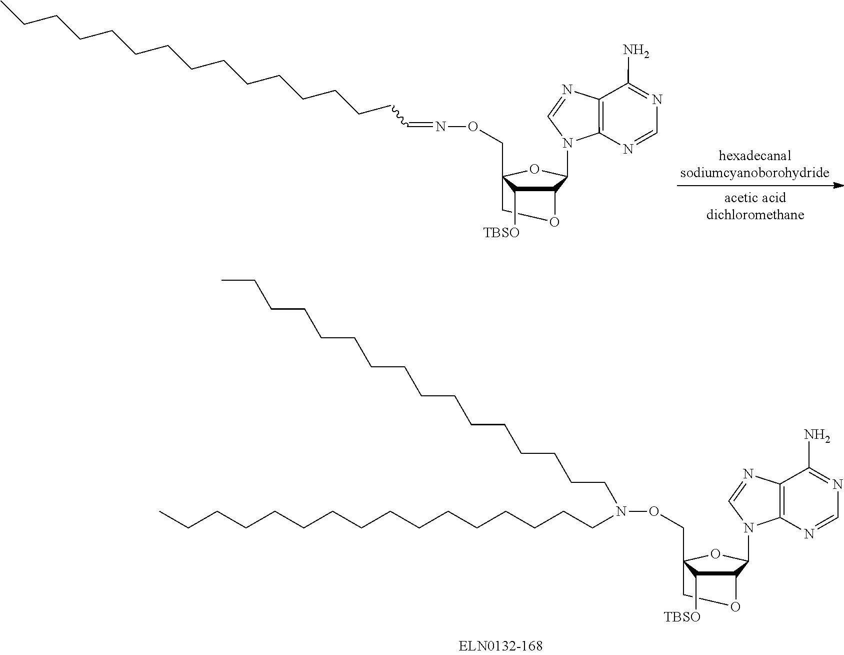 [SMILES: CCCCCCCCCCCCCCCC=NOC[C@]12CO[C@@H](C1O[Si](C)(C)C(C)(C)C)[C@H](n1cnc3c(N)ncnc31)O2 ; CCCCCCCCCCCCCCCCN(CCCCCCCCCCCCCCCC)OC[C@]12CO[C@@H](C1O[Si](C)(C)C(C)(C)C)[C@H](n1cnc3c(N)ncnc31)O2]

To a clear solution of 9-[(4R,6R)-7-[tert-butyl(dimethyl)silyl]oxy-4-[(hexadecylideneamino)oxymethyl]-2,5-dioxabicyclo[2.2.1]heptan-6-yl]purin-6-amine (0.25 g, 396.24 mol) in acetic acid (7 mL) was added sodium cyanoborohydride (66.06 mg, 1.03 mmol) in single portion and stirred at 15° C. for 1 hr. Hexadecanal (95.26 mg, 396.24 mol) was added to the reaction mixture and stirred further for 1 hr at 20° C. Finally, second portion of sodium cyanoborohydride (66.06 mg, 1.03 mmol, 98% purity) was added to the resultant turbid mixture and stirred for 2 hrs. Diluted the mixture with DCM (20 mL) and organic layer was washed with water (2×30 mL). DCM layer dried over anhydrous Na₂SO₄, filtered and the filtrate was evaporated to dryness. The crude residue was purified by combiflash chromatography to afford ELN0132-168 (0.15 g, 44% yield). HRMS calc. for C₄₉H₉₃N₆O₄Si [M+H]⁺ 857.7028, found 857.7016.

### (Z)—N′-(9-((JR,3R,4R,7S)-7-((tert-butyldimethylsilyl)oxy)-1-(((dihexadecylamino)oxy)methyl)-2,5-dioxabicyclo[2.2.1]heptan-3-yl)-9H-purin-6-yl)-N,N-dimethylformimidamide ELN0132-244

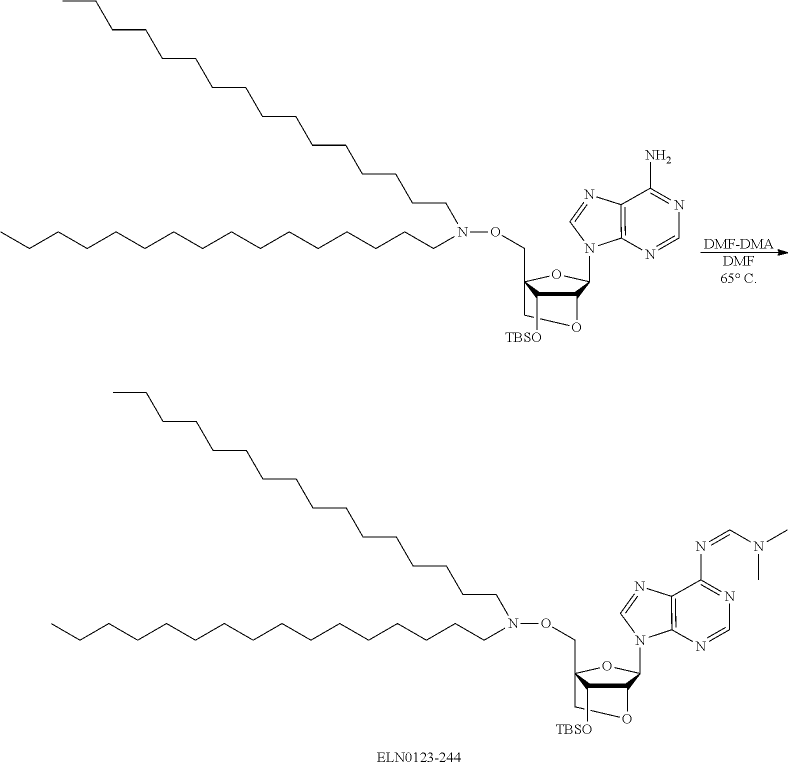

To a clear solution of ELN0132-168 (0.4 g, 466.54 mol) in dimethylformamide (5 mL) was added N,N-Dimethylformamide dimethyl acetal (88.71 mg, 699.81 mol, 99.68 μL) in single portion and the reaction mixture was stirred at 65° C. for 4 hr. TLC was checked, and volatile matters was removed under high vacuum pump. Residue was dissolved in DCM (100 mL) and the organic layer was washed with brine (3×50 mL). DCM layer was then dried over anhydrous Na₂SO₄, filtered and the filtrate was evaporated to dryness. Crude mass thus obtained, was purified by combiflash chromatography (gradient: 30-80% EtOAc in hexane) to afford ELN0132-244 (0.35 g, 82% yield) as white hygroscopic solid. HRMS calc. for C₅₂H₉₈N₇O₄Si [M+H]⁺ 912.7450, found 912.7438.

### ELN0132-16L:

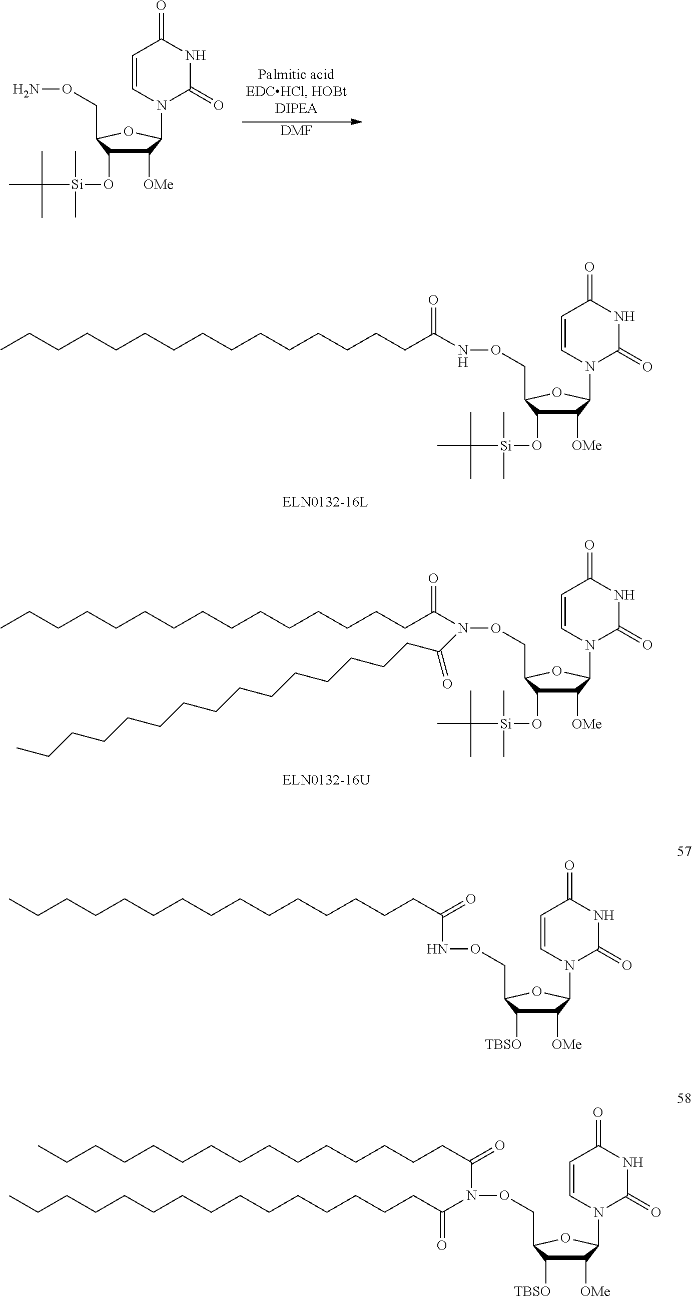

N-(((2R,3R,4R,5R)-3-((tert-butyldimethylsilyl)oxy)-5-(2,4-dioxo-3,4-dihydropyrimidin-1(2H)-yl)-4-methoxytetrahydrofuran-2-yl)methoxy)palmitamide (57, ELN0132-16 μL): To a solution of palmitic acid (160.42 mg, 619.35 umol, 188.07 uL) in dimethylformamide (3 mL), N-(3-dimethylaminopropyl)-N′-ethylcarbodiimide hydrochloride (118.73 mg, 619.35 umol) and DIPEA (134.76 mg, 1.03 mmol, 181.61 uL) were added and stirred for 10 minutes at 25° C. To this resulting reaction mixture, 1-hydroxylbenzotriazole hydrate (97.78 mg, 619.35 umol) was added and stirring was continued for 5 minutes. 14 (0.2 g, 516.13 umol) was slowly added to this resulting reaction mixture and stirred for 12 h. Two non-polar spots were observed in TLC. Reaction mixture was quenched by adding 10% NaHCO₃ solution (10 mL). Organic compounds were extracted with EtOAc (3×30 mL) from the aqueous layer. The combined organic layer was washed with brine (4×20 mL), separated, dried over anhydrous Na₂SO₄, filtered and the filtrate was evaporated to dryness. Resulting gummy residue was purified by combiflash chromatography (20-75% EtOAc in hexane) to afford 57 (0.16 g, 50% yield) and dialkylated 58 (0.15 g, 34% yield) as white hygroscopic solid.

Data for 57 ¹H NMR (400 MHz, CDCl₃) δ 9.75 (s, 1H), 9.11 (s, 1H), 7.82 (d, J=7.9 Hz, 1H), 5.95-5.51 (m, 2H), 4.37 (d, J=6.7 Hz, 1H), 4.26-3.96 (m, 3H), 3.90-3.70 (m, 1H), 3.47 (s, 3H), 2.04 (d, J=11.4 Hz, 2H), 1.61 (p, J=7.1 Hz, 2H), 1.23 (d, J=2.4 Hz, 24H), 0.87 (d, J=11.6 Hz, 12H), 0.10 (d, J=4.1 Hz, 6H) ppm. ¹³C NMR (101 MHz, CDCl₃) δ 171.34, 163.75, 150.55, 141.70, 102.65, 89.78, 83.07, 82.87, 74.81, 69.83, 58.52, 33.32, 32.03, 29.81, 29.77, 29.74, 29.60, 29.46, 29.45, 29.39, 25.80, 25.40, 22.79, 18.21, 14.22, −4.57, −4.75 ppm. HRMS calc. for C₃₂H₆₀N₃O₇Si [M+H]⁺ 626.4201, found 626.4207.

Data for N-(((2R,3R,4R,5R)-3-((tert-butyldimethylsilyl)oxy)-5-(2,4-dioxo-3,4-dihydropyrimidin-1(2H)-yl)-4-methoxytetrahydrofuran-2-yl)methoxy)-N-palmitoylpalmitamide (58, ELN0132-16U): ¹H NMR (400 MHz, CDCl₃) δ 9.86 (d, J=2.2 Hz, 1H), 7.76 (d, J=8.1 Hz, 1H), 5.91 (d, J=3.3 Hz, 1H), 5.74 (dd, J=8.1, 2.1 Hz, 1H), 4.42-4.34 (m, 1H), 4.26-4.07 (m, 4H), 3.78 (dd, J=5.0, 3.4 Hz, 1H), 3.52 (s, 3H), 2.71 (td, J=7.3, 5.6 Hz, 4H), 1.64 (p, J=7.3 Hz, 4H), 1.38-1.20 (m, 52H), 0.92 (s, 9H), 0.90-0.85 (m, 6H), 0.13 (d, J=4.0 Hz, 6H) ppm. 13C NMR (101 MHz, CDCl₃) δ 172.71, 163.60, 150.38, 140.46, 102.62, 88.79, 82.98, 82.04, 74.43, 69.98, 60.46, 58.27, 53.51, 36.67, 32.02, 29.79, 29.78, 29.75, 29.71, 29.57, 29.51, 29.45, 29.24, 25.79, 24.19, 22.78, 21.10, 18.19, 14.29, 14.20, −4.47, −4.76 ppm. LCMS calc. for C₄₈H₈₈N₃O₈Si [M−H]⁻ 862.63, found 862.6.

### [5-acetamido-3,4-dibenzoyloxy-6-[5-[[(2R,5R)-3-[tert-butyl(dimethyl)silyl]oxy-5-(2,4-dioxo pyrimidin-1-yl)-4-methoxy-tetrahydrofuran-2-yl]methoxy-hexadecanoyl-amino]-5-oxo-pent oxy]tetrahydropyran-2-yl]methyl benzoate ELN0132-34

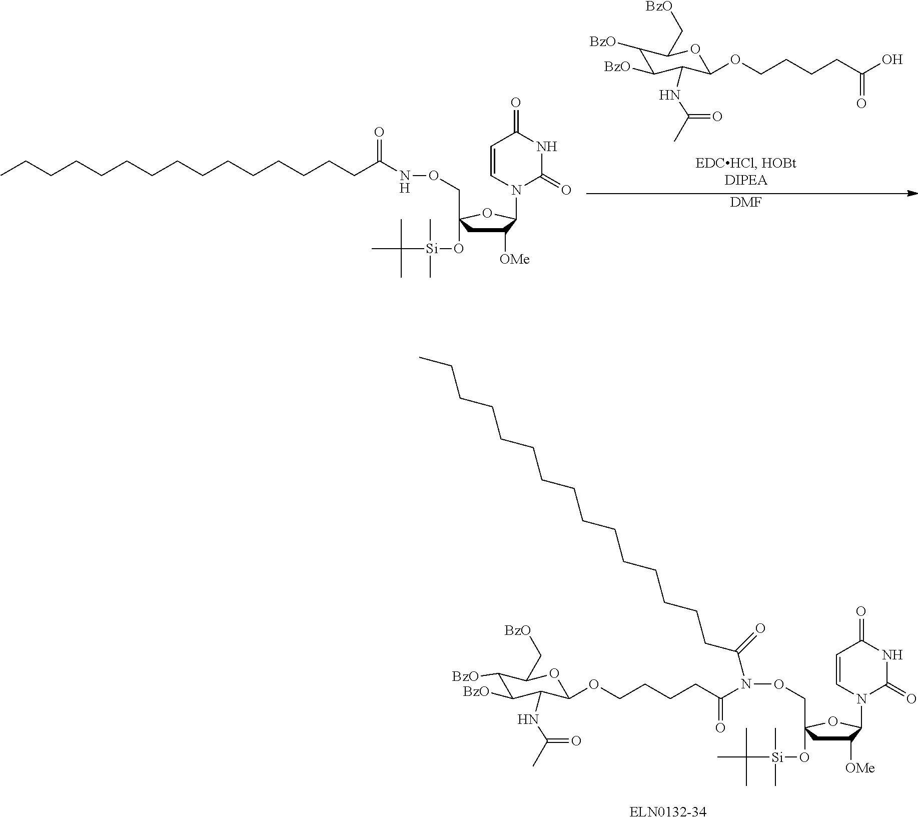

To a solution of 5-[3-acetamido-4,5-dibenzoyloxy-6-(benzoyloxymethyl)tetrahydropyran-2-yl]oxypentanoic acid (0.59 g, 0.93 mmol) in dimethylformamide (10 mL), N-(3-dimethylaminopropyl)-N′-ethylcarbodiimide hydrochloride (0.18 g, 0.93 mmol), 1-hydroxylbenzotriazole hydrate (0.074 g, 0.46 mmol) and DIPEA (0.298 mL, 1.69 mmol) were added at 25° C. To this clear solution, N-[[(2R,5R)-3-[tert-butyl(dimethyl)silyl]oxy-5-(2,4-dioxopyrimidin-1-yl)-4-methoxy-tetrahydrofuran-2-yl]methoxy]hexadecanamide (0.53 g, 0.85 mmol) was added in single portion and stirred for 18 h at 25° C. To the reaction mixture, saturated NaHCO₃ solution (20 mL) and EA (30 mL) were added. Organic layer was separated, washed again with brine (100 mL) and 10% NH₄Cl solution (30 mL). EA layer then dried over anhydrous Na₂SO₄ and filtered. Filtrate was evaporated to dryness and crude mass thus obtained, was purified by combiflash chromatography (Gradient: 10-70% EA in hexane) to afford ELN0132-34 (0.5 g, 48% yield) as white solid. ¹H NMR (500 MHz, CDCl₃, TMS, 25° C.) δ 8.83 (d, J=2.3 Hz, 1H), 8.14-8.07 (m, 2H), 8.04-7.96 (m, 2H), 7.87-7.79 (m, 2H), 7.69 (d, J=8.2 Hz, 1H), 7.64-7.58 (m, 1H), 7.57-7.52 (m, 1H), 7.51-7.44 (m, 3H), 7.41 (t, J=7.8 Hz, 2H), 7.34-7.27 (m, 2H), 5.98 (d, J=8.5 Hz, 1H), 5.89 (dd, J=3.5, 1.2 Hz, 1H), 5.87 (d, J=4.0 Hz, 1H), 5.77 (dd, J=8.1, 2.3 Hz, 1H), 5.69 (dd, J=11.2, 3.4 Hz, 1H), 4.89 (d, J=8.3 Hz, 1H), 4.65 (dd, J=11.2, 6.4 Hz, 1H), 4.45-4.24 (m, 4H), 4.24-4.14 (m, 3H), 3.99 (dt, J=9.5, 5.7 Hz, 1H), 3.85 (dd, J=5.1, 4.0 Hz, 1H), 3.59 (dt, J=9.6, 6.1 Hz, 1H), 3.48 (s, 3H), 2.83-2.61 (m, 4H), 1.87 (s, 3H), 1.68 (ddt, J=40.7, 22.1, 7.2 Hz, 6H), 1.38-1.15 (m, 25H), 0.92 (s, 9H), 0.88 (t, J=6.9 Hz, 3H), 0.13 (d, J=4.1 Hz, 6H) ppm. ¹³C NMR (101 MHz, CDCl₃,25° C.) δ 172.66, 172.44, 170.61, 166.04, 166.01, 165.73, 163.70, 150.30, 140.79, 133.45, 133.26, 133.16, 130.01, 129.82, 129.73, 129.54, 129.29, 129.07, 128.57, 128.41, 128.32, 102.55, 101.24, 88.93, 82.59, 82.10, 74.55, 71.16, 71.04, 69.96, 69.17, 68.01, 62.33, 58.15, 51.78, 36.54, 36.24, 31.90, 29.67, 29.64, 29.60, 29.47, 29.42, 29.33, 29.15, 28.78, 25.70, 24.08, 23.28, 22.67, 20.71, 18.08, 14.11, −4.58, -4.81 ppm. HRMS calc. for C₆₆H₉₃N₄O₁₇Si [M+H]⁺ 1241.6305, found 1241.6272.

### Synthesis of Aminooxy Ligands:

### Synthesis of Short Lipophilic Peptide Aldehyde for AOCC Conjugation:

### (2S)-2-[[(2S)-2-(hexadecanoylamino)-3-phenyl-propanoyl]amino]-3-phenyl-propanoic acid ELN0132-295

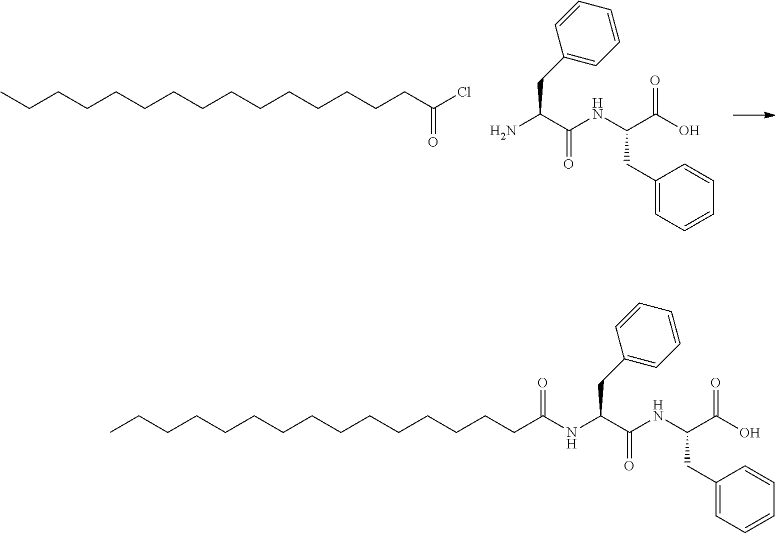 [SMILES: CCCCCCCCCCCCCCCC(=O)Cl ; N[C@@H](Cc1ccccc1)C(=O)N[C@@H](Cc1ccccc1)C(=O)O ; CCCCCCCCCCCCCCCC(=O)N[C@@H](Cc1ccccc1)C(=O)N[C@@H](Cc1ccccc1)C(=O)O]

To a clear solution of (2S)-2-[[(2S)-2-amino-3-phenyl-propanoyl]amino]-3-phenyl-propanoic acid (1.0 g, 3.20 mmol) 60 mL of 1 M NaOH in a round-bottomed flask was added hexadecanoyl chloride (879.96 mg, 3.20 mmol) at 0° C. The mixture was stirred for 16 hr. The suspension was acidified with 14 mL of 5 M HCl. The residue obtained after filtration was washed with water and diethyl ether and dried to afford ELN0132-295 (1.5 g, 85.07% yield) as white solid. ¹H NMR (500 MHz, DMSO) δ 8.03 (d, J=8.7 Hz, 1H), 7.89 (s, 1H), 7.30-7.17 (m, 6H), 7.20-7.09 (m, 5H), 4.52-4.37 (m, OH), 4.28 (d, J=6.3 Hz, 1H), 3.08 (d, J=5.1 Hz, 1H), 3.03-2.90 (m, 2H), 2.67 (dd, J=13.9, 10.6 Hz, 1H), 2.18 (t, J=7.4 Hz, 1H), 1.96 (td, J=7.3, 3.3 Hz, 2H), 1.47 (t, J=7.2 Hz, 2H), 1.23 (s, 29H), 0.88-0.82 (m, 3H) ppm. ¹³C NMR (101 MHz, DMSO) δ 174.48, 172.70, 171.91, 171.05, 138.19, 137.92, 129.32, 129.12, 127.93, 127.85, 126.11, 126.00, 53.99, 53.76, 37.35, 36.77, 35.19, 33.72, 31.28, 29.04, 29.00, 28.97, 28.90, 28.88, 28.79, 28.74, 28.69, 28.55, 28.42, 25.14, 24.51, 22.08, 13.93 ppm.

Reference: ChemNanoMat, 2018, 4, 796-800, content of which is incorporated herein by reference in its entirety.

### N-[(1S)-1-benzyl-2-[[(1S)-1-benzyl-2-(4,4-diethoxybutylamino)-2-oxo-ethyl]amino]-2-oxo-ethyl]hexadecanamide ELN0132-298

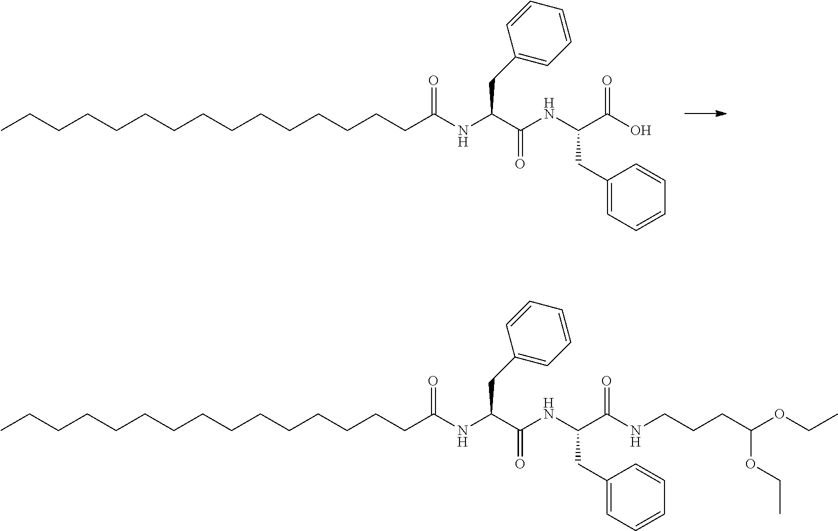 [SMILES: CCCCCCCCCCCCCCCC(=O)N[C@@H](Cc1ccccc1)C(=O)N[C@@H](Cc1ccccc1)C(=O)O ; CCCCCCCCCCCCCCCC(=O)N[C@@H](Cc1ccccc1)C(=O)N[C@@H](Cc1ccccc1)C(=O)NCCCC(OCC)OCC]

To a clear solution of ELN0132-295 (1.2 g, 2.18 mmol) in dry dimethylformamide (30 mL) was added HBTU (1.01 g, 2.61 mmol), 1-hydroxylbenzotriazole hydrate (408.56 mg, 2.61 mmol) and diisopropylethylamine (1.42 g, 10.89 mmol, 1.92 mL) in single portions. Reaction mixture was stirred for 5 minutes and then was added 4-aminobutyraldehyde diethyl acetal (1.17 g, 6.54 mmol, 90% purity). Resulting mixture was stirred at 22° C. for 16 hr and then all volatile mattered was removed under high vacuum pump. Residue was diluted with DCM (30 mL) and washed with NH₄Cl solution (2×30 mL), NaHCO₃ solution (2×40 mL), water (30 mL) and brine (2×30 mL). Organic layer was separated, dried over anhydrous Na₂SO₄, filtered and the filtrate was evaporated to dryness. Solid residue thus obtained, was coevaporated with DCM (20 mL) and kept for drying overnight at 22° C. to afford ELN0132-298 (0.98 g, 64.81% yield) as yellowish white solid. ¹H NMR (400 MHz, CDCl₃) δ 7.34-7.03 (m, 10H), 7.01-6.92 (m, 1H), 6.53-6.25 (m, 1H), 6.20-5.98 (m, 1H), 5.66 (s, 1H), 4.78-4.54 (m, 1H), 4.52-4.37 (m, 2H), 3.71-3.38 (m, 5H), 3.27 (q, J=6.7 Hz, 1H), 3.19-2.89 (m, 3H), 2.20-2.03 (m, 2H), 1.70-1.38 (m, 5H), 1.36-1.11 (m, 39H), 0.92-0.84 (m, 3H) ppm. ¹³C NMR (101 MHz, CDCl₃) δ 173.78, 173.51, 173.25, 171.06, 170.84, 170.22, 136.64, 136.47, 136.42, 136.37, 129.49, 129.42, 129.39, 129.32, 128.81, 128.79, 128.74, 128.67, 127.30, 127.20, 127.13, 127.03, 102.81, 102.63, 61.57, 61.36, 61.33, 61.26, 55.50, 54.51, 54.43, 54.25, 39.47, 39.43, 39.29, 38.36, 38.15, 37.77, 37.06, 36.52, 36.39, 32.04, 31.16, 31.01, 30.96, 29.81, 29.79, 29.77, 29.74, 29.62, 29.50, 29.47, 29.46, 29.33, 25.96, 25.62, 25.60, 24.73, 24.58, 24.53, 22.80, 15.47, 15.45, 14.23 ppm.

### Hydrolysable Aldehyde for Post-Synthetic AOCC:

### [(11Z,14Z)-1-[(10Z,13Z)-octadeca-10,13-dienyl]nonadeca-11,14-dienyl]-4-(4,4-diethoxybutyl-amino)butanoate ELN0132-503

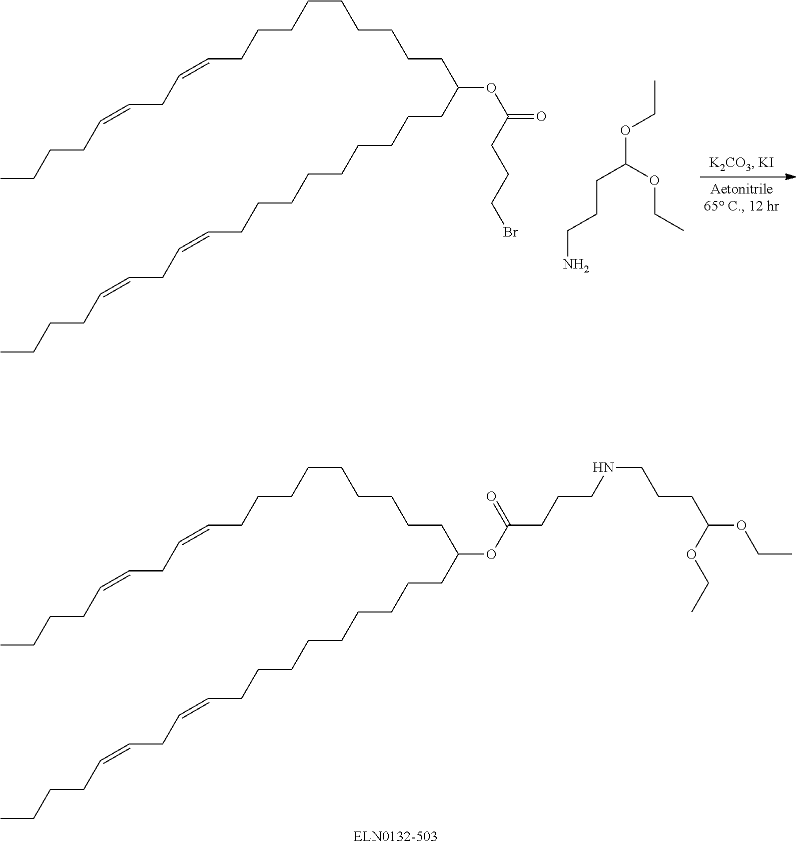 [SMILES: CCCC/C=C\C/C=C\CCCCCCCCCC(CCCCCCCCC/C=C\C/C=C\CCCC)OC(=O)CCCBr ; CCOC(CCCN)OCC ; CCCC/C=C\C/C=C\CCCCCCCCCC(CCCCCCCCC/C=C\C/C=C\CCCC)OC(=O)CCCNCCCC(OCC)OCC]

To a clear solution of 4,4-diethoxybutan-1-amine (1.33 g, 8.26 mmol) in acetonitrile (50 mL) was added potassium carbonate (570.85 mg, 4.13 mmol) and potassium iodide (68.56 mg, 413.03 mol) in single portions. Reaction mixture was stirred for 10 minutes and then [(11Z,14Z)-1-[(10Z,13Z)-octadeca-10,13-dienyl]nonadeca-11,14-dienyl]₄-bromobutanoate (1.4 g, 2.07 mmol) was added dropwise into it. Reaction mixture was stirred at 65° C. for 12 hr until the TLC showed consumption of starting material. All the volatile matters were removed under high vacuum pump. Solid residue was dissolved in DCM (40 mL) and organic layer was washed with water (40 mL) and NH₄Cl solution (40 mL). Organic layer was separated, dried over anhydrous Na₂SO₄, filtered and the filtrate was evaporated to dryness. The residue was purified by flash column chromatography (gradient: 0-5% MeOH in DCM) to afford ELN0132-503 (1.08 g, 69% yield) as yellow transparent oil. ¹H NMR (600 MHz, CDCl₃) δ 5.42-5.29 (m, 7H), 4.91-4.80 (m, 1H), 4.49 (t, J=5.6 Hz, 1H), 3.64 (dq, J=9.5, 7.0 Hz, 2H), 3.49 (dq, J=9.4, 7.0 Hz, 2H), 2.80-2.73 (m, 4H), 2.67-2.59 (m, 4H), 2.34 (t, J=7.5 Hz, 2H), 2.08-1.99 (m, 8H), 1.80 (p, J=7.4 Hz, 2H), 1.68-1.60 (m, 2H), 1.60-1.44 (m, 7H), 1.37-1.25 (m, 35H), 1.20 (t, J=7.1 Hz, 6H), 0.89 (t, J=6.9 Hz, 6H) ppm. ¹³C NMR (151 MHz, CDCl₃) δ 173.48, 130.30, 130.25, 128.07, 128.03, 102.89, 74.42, 61.11, 49.77, 49.29, 34.24, 32.63, 31.65, 31.63, 31.52, 29.81, 29.79, 29.71, 29.68, 29.66, 29.63, 29.60, 29.47, 29.44, 29.42, 27.35, 27.31, 25.74, 25.68, 25.50, 25.47, 22.70, 15.49, 15.46, 14.22 ppm.

### Synthesis of Compound 20 and Compound 21

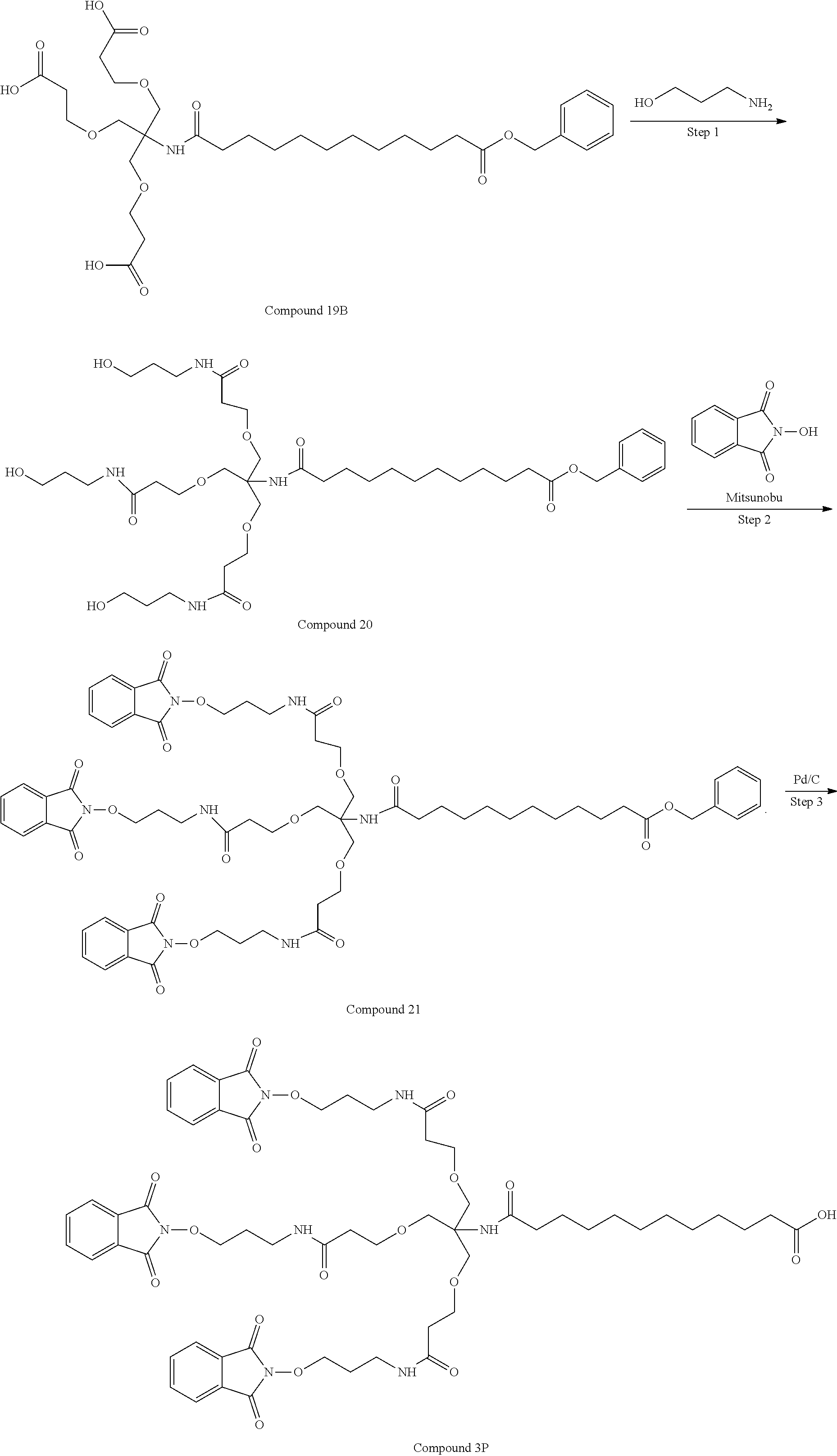

Compound 20: To a stirred solution of compound-191B (100.0 g, 0.1563 mol.) in DCM (1.0 L, 10 vol.) was added solution of 3-amino-1-propanol (38.74 g, 0.5158 mol.) in DCM (500.0 mL, 5.0 vol.) at 0-5° C. To the above mixture were added DIPEA (272.2 mL, 1.563 mol) at 0-5° C. followed by HATU (196.11 g, 0.5157 mol.) at the same temperature and the resulting reaction mixture was stirred for 12 h at room temperature. The progress of the reaction was monitored by TLC using 1:7 MeOH: DCM as eluent. Reaction mixture was diluted with DCM (5.0 vol.) and the combined organic layers were washed with brine (10.0 vol.), dried over anhydrous sodium sulfate, filtered and filtrate was evaporated under reduced pressure to get the desired crude. Further obtained crude was purified by column chromatography using 15% MeOH: DCM as eluent to afford 20 as pale yellow semisolid (93.0 g, yield: 73.36%). ¹H NMR (300 MHz, DMSO-d6): δ 7.83 (t, J=5.4 Hz, 3H), 7.30-7.40 (m, 5H), 6.97 (bs, 1H), 5.07 (s, 2H), 4.42 (t, J=5.4 Hz, 3H), 3.52-3.55 (m, 12H), 3.33-3.42 (m, 6H), 3.05-3.11 (m, 6H), 2.24-2.36 (m, 8H), 2.05 (t, J=7.2 Hz, 2H), 1.42-1.57 (m, 10H), 1.21 (bs, 12H) ppm.

Compound 21: A stirred solution of 20 (90.0 g, 0.11 mol) in THF (900.0 mL, 10.0 vol.) was added N-hydroxyl phthalimide (181.02 g, 1.10 mol.) at room temperature. To the above solution, triphenyl phosphine (291.06 g, 1.10 mol.) was added at 0-5° C. and the reaction mixture was stirred at the same temperature for 10 minutes. Then added DIAD (283.41 mL, 1.1097 Mol.) dropwise over the period of 30 minutes at 0-5° C. and the resulting reaction mixture was warmed to room temperature for 12 h. The progress of the reaction was monitored by TLC using 10% MeOH: EtOAc as eluent. Upon completion of the starting material, reaction mixture was quenched with water (10.0 vol.) and extracted with ethyl acetate (5.0 vol.). The combined organic layers were washed with aqueous saturated sodium bicarbonate solution (10.0 vol.) followed by brine solution (10.0 vol.), dried over anhydrous sodium sulfate, filtered and filtrate was evaporated under reduced pressure to get the desired crude. Further obtained crude was purified by reverse phase column chromatography using 0.01% Ammonium acetate in Water-Acetonitrile mobile phase to afford 21 as off white semisolid. (25.5 g, yield: 18.43%). ¹H NMR (400 MHz, DMSO-d6): δ 7.87-7.90 (m, 3H), 7.83-7.84 (m, 12H), 7.30-7.35 (m, 5H), 6.95 (bs, 1H), 5.05 (s, 2H), 4.13 (t, J=6.4 Hz, 6H), 3.51-3.55 (m, 12H), 3.18-3.23 (m, 6H), 2.26-2.32 (m, 8H), 2.01-2.04 (m, 2H), 1.76-1.79 (m, 6H), 1.35-1.48 (m, 4H), 1.14-1.17 (m, 12H) ppm. Mass Report of compound 21 Compound m/z=1246.35: Observed m/z+1=1247.

Synthesis of Compound 3P: To a stirred solution of 21 (25.0 g, 0.020 mol.) in IPA-THF (2:1 ratio) (200 mL, 8.0 vol.) was charged 10% Palladium on Carbon (2.5 g, 10% weight/weight) and the resulting suspension was subjected to hydrogenation (20 kg/cm²H₂ pressure) at room temperature for 12 h. The progress of the reaction was monitored by TLC using 10% methanol: ethyl acetate as eluent. Upon completion of the staring material, the reaction mixture was filtered through celite bed and filtrate was concentrated under reduced pressure at 40° C. The residue obtained was triturated with DCM: Hexane (0.5:10 ratio) (10.0 vol.), decanted the top layer and residue was dried under reduced pressure to afford 3P as off-white solid (22.0 g, yield: 94.86%). ¹H NMR (400 MHz, DMSO-d₆) δ 11.96 (s, 1H), 7.91 (t, J=5.5 Hz, 3H), 7.84 (s, 12H), 6.96 (s, 1H), 4.15 (t, J=6.4 Hz, 6H), 3.54 (dd, J=11.0, 4.6 Hz, 12H), 3.22 (q, J=6.6 Hz, 6H), 2.28 (t, J=6.3 Hz, 6H), 2.15 (t, J=7.4 Hz, 2H), 2.04 (t, J=7.4 Hz, 2H), 1.81 (p, J=6.7 Hz, 6H), 1.42 (d, J=9.0 Hz, 4H), 1.20-1.15 (m, 13H) ppm. ¹³C NMR (101 MHz, DMSO-d₆) δ 174.50, 172.46, 170.22, 163.31, 134.77, 128.57, 123.22, 75.75, 68.28, 67.29, 59.44, 35.96, 35.92, 35.28, 33.65, 28.98, 28.94, 28.86, 28.77, 28.65, 28.57, 28.05, 25.31, 24.50 ppm. Compound m/z=1156.23): Observed m/z+1=1156.8

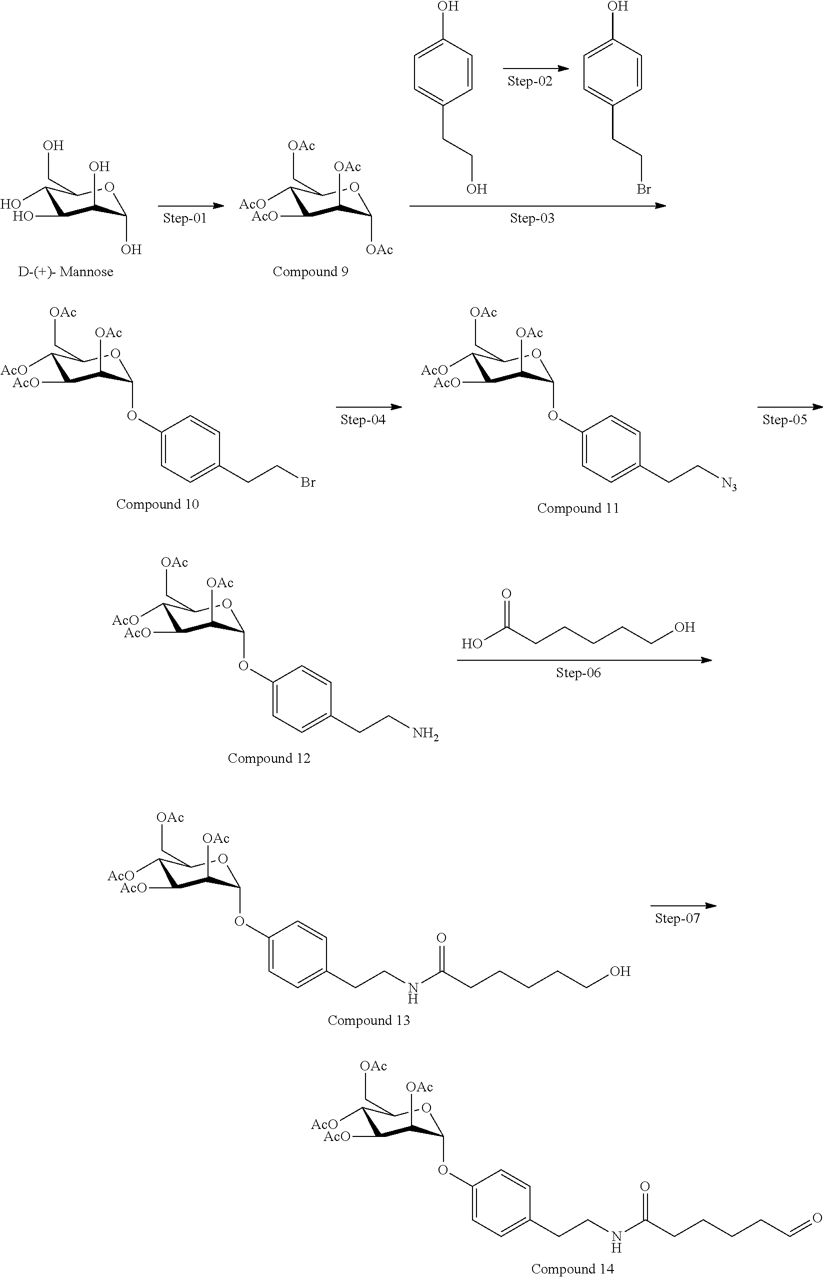

Synthesis of compound 9: To a cooled and stirred solution of D-(+)-Mannose (20.0 g, 0.11 Mol.) in Acetic anhydride (75.6 g, 3.78 w/w) was added pyridine (95 mL, 4.75 vol.) dropwise over the period of 15 minutes at 0-5° C. To the above suspension was added DMAP (1.24 g, 0.01 Mol.) followed by triethylamine (15.49 mL, 0.11 Mol.) at 0-5° C. (exothermic up to 40° C.) and the resulting reaction mixture was stirred at room temperature for 12 h. The progress of the reaction was monitored by TLC using 10% MeOH: DCM as eluent. Reaction mixture was concentrated under reduced pressure. Residue obtained was dissolved in DCM (200 mL) and washed with 10% aqueous sodium bicarbonate solution (2×100 mL). The combined organic layers were dried over anhydrous sodium sulfate, filtered and filtrate was evaporated under reduced pressure to afford [(3R,6R)-3,4,5,6-tetrakis(acetyloxy)oxan-2-yl]methyl acetate as white semisolid. (42.05 g, Yield: 97.04%). ¹H NMR (300 MHz, DMSO-d6): δ 5.96 (s, 1H), 5.16-5.35 (m, 3H), 4.12-4.19 (m, 2H), 3.99-4.06 (m, 2H), 2.13-2.15 (s, 6H), 1.94-2.07 (s, 9H) ppm.

### Synthesis of 4-(2-bromoethyl)phenol

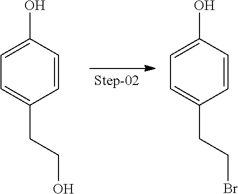 [SMILES: OCCc1ccc(O)cc1 ; Oc1ccc(CCBr)cc1]

A stirred solution of 4-(2-Hydroxyl-ethyl)-phenol (10.0 g, 0.07 Mol) in aqueous HBr (48% in water) (50.0 mL, 5.0 vol.) was heated at 80° C. for 3 h. The progress of the reaction was monitored by TLC using 50% EtOAc: Hexane as eluent. Upon completion of the starting material, reaction mixture was cooled to room temperature then diluted with DCM (10.0 vol.). Later organic layer was washed with 10% aqueous sodium bicarbonate solution (2×5.0 vol.). The combined organic layers were washed dried over anhydrous sodium sulfate, filtered and filtrate was evaporated under reduced pressure afford 4-(2-bromoethyl)phenol as white solid. (11.25 g, Yield: 76.21%). ¹H NMR (300 MHz, DMSO-d6): δ 9.27 (s, 1H), 7.05 (d, J=8.4 Hz, 2H), 6.69 (d, J=8.7 Hz, 2H), 3.62 (d, J=7.5 Hz, 2H), 2.99 (t, J=7.2 Hz, 2H),

### Synthesis of Compound-10

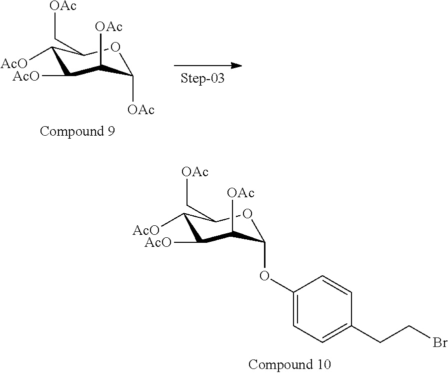

A stirred solution of [(3R,6R)-3,4,5,6-tetrakis(acetyloxy)oxan-2-yl]methyl acetate (30.0 g, 0.07 mol.) in 1,2-dichloroethane (300.0 mL, 10.0 vol.) were added 4-(2-bromoethyl)phenol (13.90 g, 0.06 mol.) and molecular sieves (30.0 g, w/w) at room temperature. The above mixture was cooled to 5-10° C. then added Trimethylsilyl trifluoromethanesulfonate (34.77 mL, 0.19 mol.) dropwise over the period of 15 minutes at 10-15° C. The resulting reaction mixture was warmed to room temperature for 3 h. The progress of the reaction was monitored by TLC using 50% Ethyl acetate-Hexane as eluent. Upon completion of the staring material, reaction mixture filtered through celite bed. Filtrate was added dropwise to 10% aqueous sodium bicarbonate solution (6.0 vol.) at 10° C. to 15° C. Separated the organic layer, dried over anhydrous sodium sulfate, filtered and filtrate was evaporated under reduced pressure to get the desired crude. Further obtained crude was purified by silica gel chromatography using 50% Ethyl acetate-Hexane as eluent to [(3R,6R)-3,4,5-tris(acetyloxy)-6-[4-(2-bromoethyl)phenoxy]oxan-2-yl]methyl acetate as white solid (26.5 g, yield: 64.9%). ¹H NMR (400 MHz, DMSO-d6): δ 7.24 (d, J=8.8 Hz, 2H), 7.07 (d, J=8.2 Hz, 2H), 5.69 (s, 1H), 5.29-5.32 (m, 2H), 5.13-5.18 (m, 1H), 4.11-4.16 (m, 1H), 4.02-4.07 (m, 1H), 3.93-3.97 (m, 1H), 3.68 (t, J=7.2 Hz, 2H), 3.06 (t, J=7.2 Hz, 2H), 2.13 (s, 3H), 2.02 (s, 3H), 1.96 (s, 3H), 1.90 (s, 3H).

### Synthesis of Compound 11

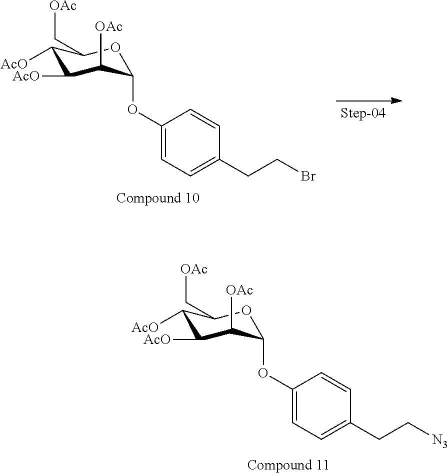

A stirred solution of [(3R,6R)-3,4,5-tris(acetyloxy)-6-[4-(2-bromoethyl)phenoxy]oxan-2-yl]methyl acetate (40.0 g, 0.075 mol.) in DMF (400.0 mL, 10.0 vol.) were added sodium azide (14.6 g, 0.22 mol.) followed by sodium iodide (33.8 g, 0.22 Mol.) and the resulting suspension was stirred for 48 h at room temperature. The progress of the reaction was monitored by TLC using 30% Ethyl acetate: Hexane as eluent. Upon completion of the staring material, the reaction mixture was quenched with ice-water (20.0 vol.) and the solid precipitated out were filtered through Buchner funnel, dried under vacuum pressure to afford [(3R,6R)-3,4,5-tris(acetyloxy)-6-[4-(2-azidoethyl)phenoxy]oxan-2-yl]methyl acetate as white solid (35.5 g, yield: 95.58%). ¹H NMR (400 MHz, DMSO-d6): δ 7.23 (d, J=8.4 Hz, 2H), 7.07 (d, J=8.8 Hz, 2H), 5.69 (s, 1H), 5.29-5.32 (m, 2H), 5.15-5.18 (m, 1H), 4.11-4.15 (m, 1H), 4.03-4.06 (m, 1H), 3.92-3.96 (m, 1H), 3.52 (t, J=7.2 Hz, 2H), 2.79 (t, J=6.8 Hz, 2H), 2.13 (s, 3H), 2.02 (s, 3H), 1.96 (s, 3H), 1.90 (s, 3H) ppm. Mass Report of compound 11 Compound m/z=493.46): Observed m/z+18=511.3

### Synthesis of Compound 12: [(3R,6R)-3,4,5-tris(acetyloxy)-6-[4-(2-aminoethyl)phenoxy]oxan-2-yl]methyl acetate (A012102525)

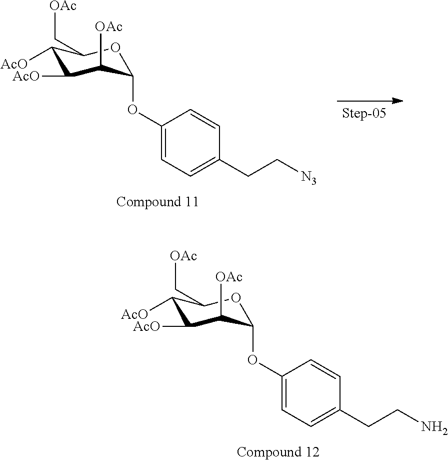

A stirred solution of benzyl [(3R,6R)-3,4,5-tris(acetyloxy)-6-[4-(2-azidoethyl)phenoxy]oxan-2-yl]methyl acetate (25.0 g, 0.050 mol.) in IPA-THF (1:1 ratio) (250.0 mL, 10.0 vol.) were charged p-toluene sulfonic acid monohydrate (8.61 g, 0.050 mol.) and 10% Palladium on Carbon (5.0 g, 20% weight/weight) and the resulting suspension was subjected to hydrogenation (1 kg/cm²H₂ pressure) at room temperature for 12 h. The progress of the reaction was monitored by TLC using 10% Methanol:DCM as eluent. Upon completion of the staring material, the reaction mixture was filtered through celite bed and filtrate was concentrated under reduced pressure at 35° C. to afford [(3R,6R)-3,4,5-tris(acetyloxy)-6-[4-(2-aminoethyl)phenoxy]oxan-2-yl]methyl acetate as Off-white solid (20.5 g, yield: 86.57%). ¹H NMR (400 MHz, DMSO-d6): δ 7.45-7.49 (m, 3H), 7.21 (d, J=8.8 Hz, 2H), 7.10 (d, J=8 Hz, 2H), 7.08-7.09 (m, 4H), 5.68 (s, 1H), 5.29-5.32 (m, 2H), 5.17-5.19 (m, 1H), 4.12-4.15 (m, 1H), 3.96-3.97 (m, 1H), 3.93-3.94 (m, 1H), 2.98-3.0 (m, 2H), 2.77-2.81 (m, 2H), 2.27 (s, 3H), 2.13 (s, 3H), 2.02 (s, 3H), 1.96 (m, 3H), 1.91 (s, 3H) ppm. Mass Report of Compound 12 Compound m/z=467.46): Observed m/z+1=468.3

### Synthesis of Compound 13

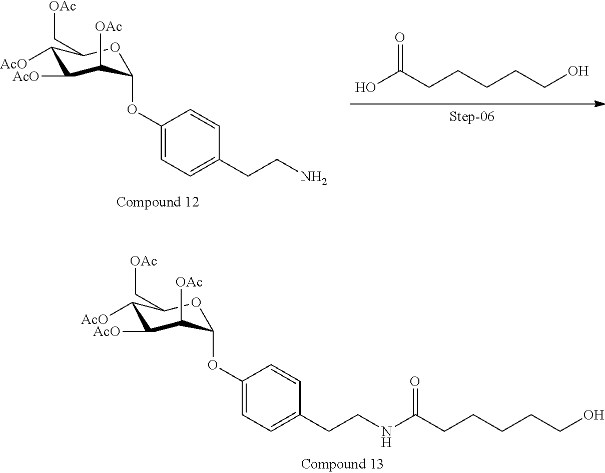

A stirred solution of 6-Hydroxyl-hexanoic acid (6.20 g, 0.046 mol.) in DCM (100 mL, 5.0 vol.) was HOBt (0.42 g, 0.003 mol.) at room temperature. To the above mixture, EDC·HCl (11.99 g, 0.006 mol.) and DIPEA (32.7 mL, 0.18 mol.) were added at 0-5° C. and the resulting reaction mixture was stirred for 15 minutes at 0 −5° C. Then solution of [(3R,6R)-3,4,5-tris(acetyloxy)-6-[4-(2-aminoethyl)phenoxy]oxan-2-yl]methyl acetate (20.0 g, 0.031 mol.) in DCM (100.0 mL, 5.0 vol.) was added over the period of 10 minutes at 0° C.±5° C. and the reaction mixture was stirred at room temperature for 12 h. The progress of the reaction was monitored by TLC using 5% methanol:DCM as eluent. Upon completion of the staring material, the reaction mixture was quenched with water (5.0 vol.) and extracted with DCM (2.5 vol.). The combined organic layer was washed water (5.0 vol.) and brine solution (5.0 vol.), dried over anhydrous sodium sulfate, filtered and filtrate was evaporated under reduced pressure to get the desired crude. Further obtained crude was purified by silica gel chromatography using 5% Methanol:DCM as eluent to afford 13 as off white solid (15.5 g, yield: 85.21%). ¹H NMR (600 MHz, DMSO-d6) δ 7.85 (t, J=5.6 Hz, 1H), 7.20-7.11 (m, 2H), 7.10-6.99 (m, 2H), 5.67 (d, J=1.5 Hz, 1H), 5.35-5.28 (m, 2H), 5.22-5.10 (m, 1H), 4.35 (t, J=5.1 Hz, 1H), 4.16 (dd, J=12.3, 5.5 Hz, 1H), 4.06 (ddd, J=10.2, 5.5, 2.4 Hz, 1H), 3.96 (dd, J=12.3, 2.4 Hz, 1H), 3.40-3.33 (m, 3H), 3.22 (dt, J=7.9, 6.3 Hz, 2H), 2.65 (t, J=7.4 Hz, 2H), 2.14 (s, 3H), 2.03 (d, J=11.0 Hz, 5H), 1.98 (s, 3H), 1.92 (s, 3H), 1.45 (q, J=7.6 Hz, 2H), 1.41-1.35 (m, 2H), 1.26-1.19 (m, 2H) ppm. ¹³C NMR (151 MHz, DMSO-d6) δ 172.06, 169.96, 169.75, 169.69, 169.59, 153.52, 134.22, 129.78, 116.98, 95.45, 68.69, 68.54, 68.48, 65.24, 61.71, 60.66, 40.21, 35.53, 34.42, 32.36, 25.29, 25.27, 20.70, 20.49, 20.46 ppm.

### Synthesis of Compound 14

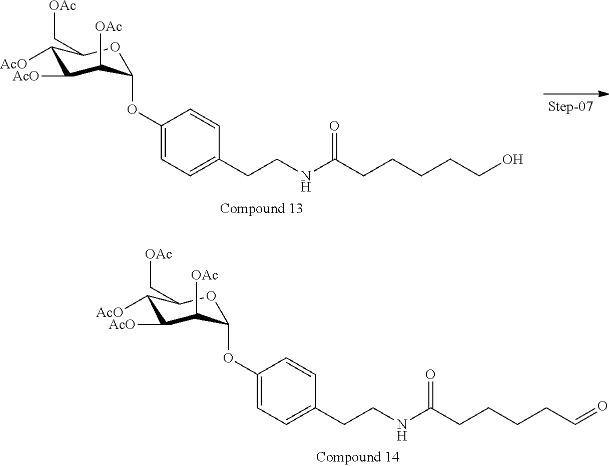

A stirred cold solution of Oxalyl chloride (11.05 mL, 0.12 mol.) in DCM (30.0 mL, 2.0 vol.) was added DMSO (18.25 mL, 0.25 mol.) dropwise at −78° C. and the resulting white suspension was stirred for 1.5 h at −78° C. Then solution of 13 (15.0 g, 0.025 mol.) in DCM (45.0 mL, 3.0 vol.) was added dropwise over the period of 15 minutes at −78° C. and stirred for an additional 2 h at the same temperature. After that triethylamine (54.17 mL, 0.38 mol.) was added over the period of 10 minutes at −78° C. and stirred for 30 minutes at −78° C. later warmed to room temperature for an additional 30 minutes. The progress of the reaction was monitored by TLC using 5% methanol:DCM as eluent. Upon completion of the staring material, the reaction mixture was quenched with water (10.0 vol.) and extracted with DCM (6.0 vol.). The combined organic layers were washed with water (2×10.0 vol.) followed by brine (10.0 vol.), dried over anhydrous sodium sulfate, filtered and filtrate was evaporated under reduced pressure to get the desired crude. Further obtained crude was triturated with DCM (0.5 vol.)-Hexane (10.0 vol.), decanted the top layer, residue was dried under reduced pressure to afford, 14 as brown semisolid (14.0 g, yield: 93.7%). ¹H NMR (600 MHz, DMSO-d₆) δ 9.64 (s, 1H), 7.88 (t, J=5.8 Hz, 1H), 7.16 (d, J=8.1 Hz, 2H), 7.05 (d, J=8.1 Hz, 2H), 5.76 (s, 1H), 5.66 (s, 1H), 5.32 (d, J=6.8 Hz, 2H), 5.17 (t, J=10.3 Hz, 1H), 4.15 (dd, J=12.3, 5.5 Hz, 1H), 4.09-4.03 (m, 1H), 3.96 (d, J=12.2 Hz, 1H), 3.22 (q, J=6.9 Hz, 2H), 2.65 (t, J=7.4 Hz, 2H), 2.41 (d, J=6.5 Hz, 1H), 2.14 (s, 3H), 2.04 (s, 4H), 1.98 (s, 3H), 1.92 (s, 3H), 1.45 (s, 4H) ppm. ¹³C NMR (151 MHz, DMSO-d₆) δ 203.45, 171.76, 169.95, 169.75, 169.68, 169.58, 153.51, 134.18, 129.78, 116.97, 95.44, 68.67, 68.52, 68.46, 65.22, 61.69, 54.97, 42.78, 40.16, 35.14, 34.38, 24.81, 21.18, 20.70, 20.49, 20.46 ppm. Mass Report of Compound 14 Compound m/z=579.59): Observed m/z+1=580.3

### REFERENCES

1. Scaranti, M.; Cojocaru, E.; Banerjee, S.; Banerji, U., Exploiting the folate receptor α in oncology. Nature Reviews Clinical Oncology 2020, 17(6), 349-359.
2. Kolmel, D. K.; Kool, E. T., Oximes and Hydrazones in Bioconjugation: Mechanism and Catalysis. Chem Rev 2017, 117 (15), 10358-10376.
3. Meyer, A.; Vasseur, J.-J.; Dumy, P.; Morvan, F., Phthalimide-Oxy Derivatives for 3′- or 5′-Conjugation of Oligonucleotides by Oxime Ligation and Circularization of DNA by “Bis- or Tris-Click” Oxime Ligation. European Journal of Organic Chemistry 2017, 2017 (46), 6931-6941.
4. Noel, M.; Clement-Blanc, C.; Meyer, A.; Vasseur, J. J.; Morvan, F., Solid Supports for the Synthesis of 3′-Aminooxy Deoxy- or Ribo-oligonucleotides and Their 3′-Conjugation by Oxime Ligation. J Org Chem 2019, 84 (22), 14854-14860.
5. Salo, H.; Virta, P.; Hakala, H.; Prakash, T. P.; Kawasaki, A. M.; Manoharan, M.; Lönnberg, H., Aminooxy Functionalized Oligonucleotides: Preparation, On-Support Derivatization, and Post synthetic Attachment to Polymer Support. Bioconjugate Chemistry 1999, 10 (5), 815-823.
6. Stukel, J. M.; Li, R. C.; Maynard, H. D.; Caplan, M. R., Two-Step Synthesis of Multivalent Cancer-Targeting Constructs. Biomacromolecules 2010, 11 (1), 160-167.
7. Cieslak, J.; Grajkowski, A.; Ausin, C.; Gapeev, A.; Beaucage, S. L., Permanent or reversible conjugation of 2′-O- or 5′-O-aminooxymethylated nucleosides with functional groups as a convenient and efficient approach to the modification of RNA and DNA sequences. Nucleic Acids Res 2012, 40 (5), 2312-29.
8. Prakash, T. P.; Manoharan, M.; Kawasaki, A. M.; Lesnik, E. A.; Owens, S. R.; Vasquez, G., 2′-O-{2-[N,N-(Dialkyl)aminooxy]ethyl}—Modified Antisense Oligonucleotides. Organic Letters 2000, 2 (25), 3995-3998.
9. Prakash, T. P.; Kawasaki, A. M.; Fraser, A. S.; Vasquez, G.; Manoharan, M., Synthesis of 2′-O-[2-[(N,N-Dimethylamino)oxy]ethyl] Modified Nucleosides and Oligonucleotides. The Journal of Organic Chemistry 2002, 67 (2), 357-369.
10. Marcaurelle, L. A.; Bertozzi, C. R., Chemoselective Elaboration of O-Linked Glycopeptide Mimetics by Alkylation of 3-ThioGalNAc. Journal of the American Chemical Society 2001, 123 (8), 1587-1595.
11. Rodriguez, E. C.; Marcaurelle, L. A.; Bertozzi, C. R., Aminooxy-, Hydrazide-, and Thiosemicarbazide-Functionalized Saccharides: Versatile Reagents for Glycoconjugate Synthesis. The Journal of Organic Chemistry 1998, 63 (21), 7134-7135.
12. Sun, L.; Li, P.; Amankulor, N.; Tang, W.; Landry, D. W.; Zhao, K., N-Alkoxy Analogues of 3,4,5-Trihydroxylpiperidine. The Journal of Organic Chemistry 1998, 63 (19), 6472-6475.
13. Lee, M.-r.; Lee, J.; Baek, B.-h.; Shin, I., The First Solid-Phase Synthesis of Oligomeric α-Aminooxy Peptides. Synlett 2003, 2003 (03), 0325-0328.
14. Shin, I.; Lee, M.-r.; Lee, J.; Jung, M.; Lee, W.; Yoon, J., Synthesis of Optically Active Phthaloyl d-Aminooxy Acids from 1-Amino Acids or 1-Hydroxyl Acids as Building Blocks for the Preparation of Aminooxy Peptides. The Journal of Organic Chemistry 2000, 65 (22), 7667-7675.
15. Carrasco, M. R.; Silva, 0.; Rawls, K. A.; Sweeney, M. S.; Lombardo, A. A., Chemoselective Alkylation of N-Alkylaminooxy-Containing Peptides. Organic Letters 2006, 8 (16), 3529-3532.
16. Li, X.; Wu, Y.-D.; Yang, D., α-Aminoxy Acids: New Possibilities from Foldamers to Anion Receptors and Channels. Accounts of Chemical Research 2008, 41 (10), 1428-1438.
17. Katritzky, A. R.; Avan, I.; Tala, S. R., Efficient preparation of aminoxyacyl amides, aminoxy hybrid peptides, and alpha-aminoxy peptides. J Org Chem 2009, 74 (22), 8690-4.
18. Yang, D.; Chang, X. W.; Zhang, D. W.; Jiang, Z. F.; Song, K. S.; Zhang, Y. H.; Zhu, N. Y.; Weng, L. H.; Chen, M. Q., Chiral alpha-aminoxy acid/achiral cyclopropane alpha-aminoxy acid unit as a building block for constructing the alpha N—O helix. J Org Chem 2010, 75 (14), 4796-805.
19. Vasseur, J. J.; Debart, F.; Sanghvi, Y. S.; Cook, P. D., Oligonucleosides: synthesis of a novel methylhydroxylamine-linked nucleoside dimer and its incorporation into antisense sequences. Journal of the American Chemical Society 1992, 114 (10), 4006-4007.
20. Prhavc, M.; Just*, G.; Bhat, B.; Cook, P. D.; Manoharan, M., A new approach for the synthesis of 3′-deoxy-3′-C-formyl-ribonucleosides and the synthesis of alternating methylene(methylimino) linked phosphodiester backbone oligonucleotides with 2′-OH and 2′-OMe groups. Tetrahedron Letters 2000, 41 (51), 9967-9971.
21. Sanghvi, Y. S.; Ross, B.; Bharadwaj, R.; Vasseur, J.-J., An easy access of 2′,3′-dideoxy-3′-α-C-formyl-adenosine and -guanosine analogs via stereoselective C C bond forming radical reaction. Tetrahedron Letters 1994, 35 (27), 4697-4700.
22. Yang, X.; Han, X.; Cross, C.; Bare, S.; Sanghvi, Y.; Gao, X., NMR Structure of an Antisense DNA RNA Hybrid Duplex Containing a 3′-CH2N(CH3)-O-5′ or an MMI Backbone Linker. Biochemistry 1999, 38 (39), 12586-12596.
23. Swayze, E. E.; Sanghvi, Y. S., The Synthesis of N,N′—O-Trisubstituted Hydroxylamines via a Mild Reductive Alkylation Procedure: An Improved Synthesis of the MMI Backbone. Synlett 1997, 1997 (07), 859-861.
24. Bhat, B.; Swayze, E. E.; Wheeler, P.; Dimock, S.; Perbost, M.; Sanghvi, Y. S., Synthesis of Novel Nucleic Acid Mimics via the Stereoselective Intermolecular Radical Coupling of 3′-Iodo Nucleosides and Formaldoximes1. The Journal of Organic Chemistry 1996, 61 (23), 8186-8199.
25. Morvan, F.; Sanghvi, Y. S.; Perbost, M.; Vasseur, J.-J.; Bellon, L., Oligonucleotide Mimics for Antisense Therapeutics: Solution Phase and Automated Solid-Support Synthesis of MMI Linked Oligomers. Journal of the American Chemical Society 1996, 118 (1), 255-256.
26. Gong, Y.; Peyrat, S.; Sun, H.; Xie, J., Synthesis of nucleoside aminooxy acids. Tetrahedron 2011, 67 (37), 7114-7120.
27. Sugo, T.; Terada, M.; Oikawa, T.; Miyata, K.; Nishimura, S.; Kenjo, E.; Ogasawara-Shimizu, M.; Makita, Y.; Imaichi, S.; Murata, S.; Otake, K.; Kikuchi, K.; Teratani, M.; Masuda, Y.; Kamei, T.; Takagahara, S.; Ikeda, S.; Ohtaki, T.; Matsumoto, H., Development of antibody-siRNA conjugate targeted to cardiac and skeletal muscles. J. Controlled Release 2016, 237, 1-13.
28. Spears, R. J.; Fascione, M. A., Site-selective incorporation and ligation of protein aldehydes. Org. Biomol. Chem. 2016, 14 (32), 7622-7638.
29. Zhang, L.; Wang, Z.; Wang, Z.; Luo, F.; Guan, M.; Xu, M.; Li, Y.; Zhang, Y.; Wang, Z.; Wang, W., A Simple and Efficient Method to Generate Dual Site-Specific Conjugation ADCs with Cysteine Residue and an Unnatural Amino Acid. Bioconjug Chem 2021, 32 (6), 1094-1104.
30. Clark, J. L.; Hollecker, L.; Mason, J. C.; Stuyver, L. J.; Tharnish, P. M.; Lostia, S.; McBrayer, T. R.; Schinazi, R. F.; Watanabe, K. A.; Otto, M. J.; Furman, P. A.; Stec, W. J.; Patterson, S. E.; Pankiewicz, K. W., Design, synthesis, and antiviral activity of 2′-deoxy-2′-fluoro-2′-C-methylcytidine, a potent inhibitor of hepatitis C virus replication. J Med Chem 2005, 48 (17), 5504-8.
31. Wagner, D.; Verheyden, J. P. H.; Moffatt, J. G., Preparation and synthetic utility of some organo tin derivatives of nucleosides. J. Org. Chem. 1974, 39 (1), 24-30.
32. Israel, M.; Murray, R. J., Adriamycin analogs. 1. Preparation and antitumor evaluation of 7-O—(β-D-glucosaminyl)daunomycinone and 7-O-(p-D-glucosaminyl)adriamycinone and their N-trifluoroacetyl derivatives. J. Med. Chem. 1982, 25 (1), 24-8.
33. Kumar, P.; Parmar, R. G.; Brown, C. R.; Willoughby, J. L. S.; Foster, D. J.; Babu, I. R.; Schofield, S.; Jadhav, V.; Charisse, K.; Nair, J. K.; Rajeev, K. G.; Maier, M. A.; Egli, M.; Manoharan, M., 5′-Morpholino modification of the sense strand of an siRNA makes it a more effective passenger. Chem. Commun. (Cambridge, U. K.) 2019, 55 (35), 5139-5142.

### Synthesis of Additional Monomers

General conditions: TLC was performed on Merck silica gel 60 plates coated with F₂₅₄. Compounds were visualized under UV light (254 nm) or after spraying with the p-anisaldehyde staining solution followed by heating. Flash column chromatography was performed using a Teledyne ISCO Combi Flash system with pre-packed RediSep Teledyne ISCO silica gel cartridges. All moisture-sensitive reactions were carried out under anhydrous conditions using dry glassware, anhydrous solvents, and argon atmosphere. All commercially available reagents and solvents were purchased from Sigma-Aldrich unless otherwise stated and were used as received. ESI-MS spectra were recorded on a Waters QTof Premier instrument using the direct flow injection mode. ¹H NMR spectra were recorded at 400, 500, and 600 MHz. ¹³C NMR spectra were recorded at 101, 126 and 151 MHz. ³¹P NMR spectra were recorded at 162, 202 and 243 MHz. ¹⁹F NMR spectra were recorded at 565 MHz. Chemical shifts are given in ppm referenced to the solvent residual peak (DMSO-d₆—¹H: δ at 2.50 ppm and ¹³C δ at 39.5 ppm; CDCl₃—¹H: δ at 7.26 ppm and ¹³C δ at 77.16 ppm; CD₃CN—¹H: δ at 1.94 ppm and ¹³C δ at 1.32 ppm)¹. Coupling constants are given in Hertz. Signal splitting patterns are described as singlet (s), doublet (d), triplet (t), septet (sept), broad signal (brs), or multiplet (m).

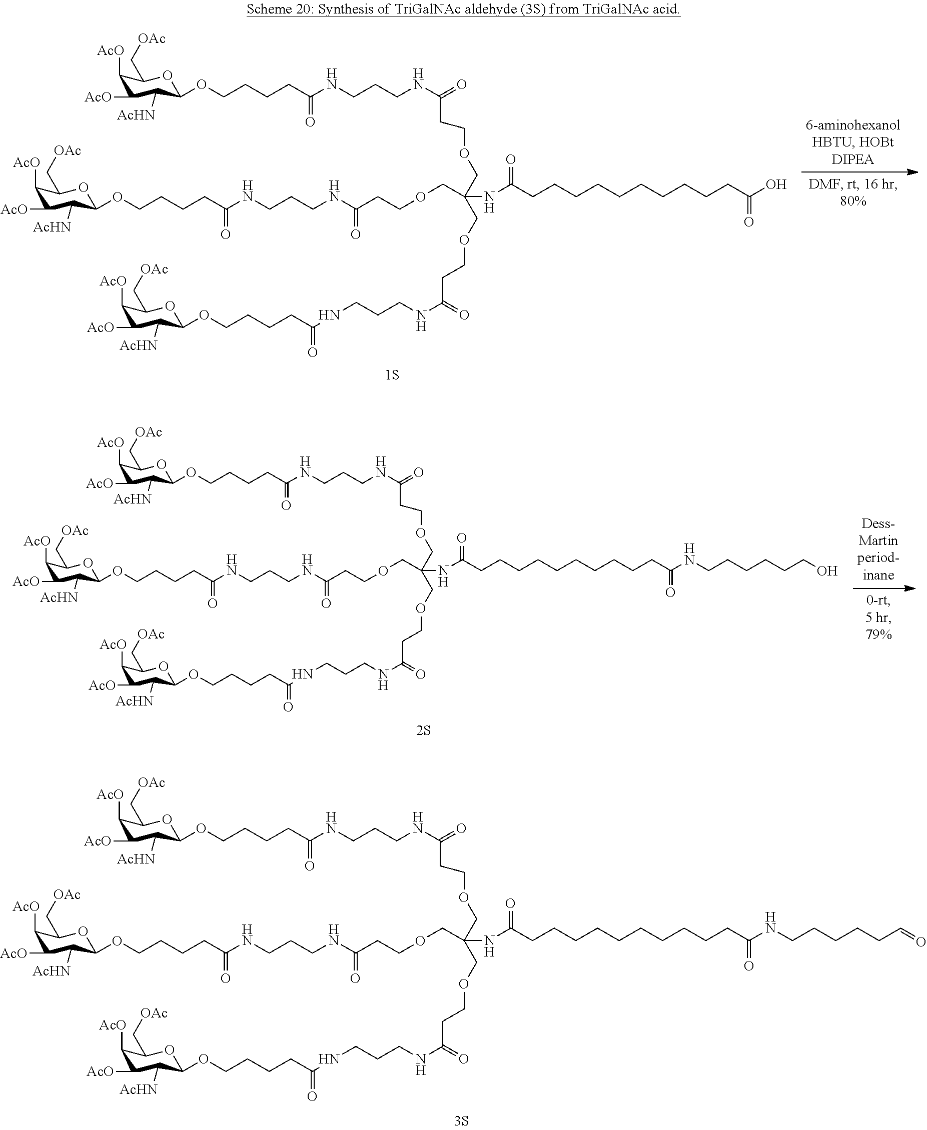

### [(3R,6R)-5-acetamido-6-[5-[3-[3-[3-[3-[3-[5-[(2R,5R)-3-acetamido-4,5-diacetoxy-6-(acetoxymethyl)tetrahydropyran-2-yl]oxypentanoylamino]propylamino-3-oxo-propoxy]-2-[[3-[3-[5-[(2R,5R)-3-acetamido-4,5-diacetoxy-6-(acetoxymethyl)tetrahydropyran-2-yl]oxypentanoylamino]propylamino]-3-oxo-propoxy]methyl]-2-[[12-(6-hydroxylhexylamino)-12-oxo-dodecanoyl]amino]propoxy]propanoylamino]propylamino]-5-oxo-pentoxy]-3,4-diacetoxy-tetrahydropyran-2-yl]methyl acetate (2S)

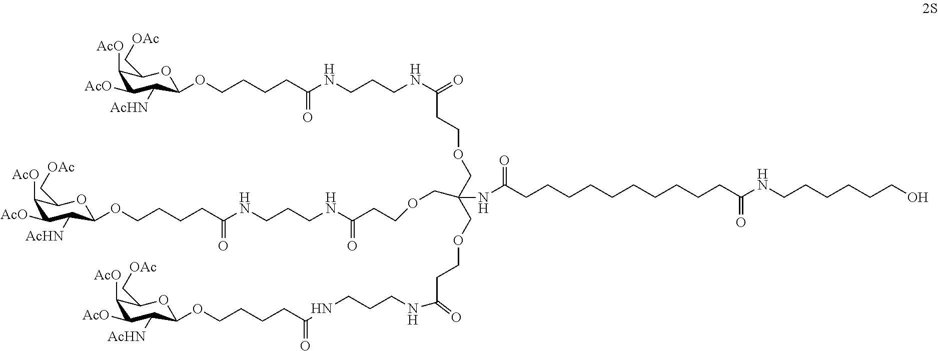

To a clear solution of TriGalNAc acid 1S³ (8.3 g, 4.14 mmol) in dry dimethylformamide (30 mL) was added HBTU (1.88 g, 4.96 mmol), HOBt (670.82 mg, 4.96 mmol) and DIPEA (1.60 g, 12.41 mmol, 2.16 mL) in single portions. Reaction mixture was stirred for 5 minutes and then was added 6-aminohexan-1-o1 (969.67 mg, 8.27 mmol) slowly. Resulting mixture was stirred at 22° C. for 16 hr and then all volatile matter was removed under high vacuum pump. Residue was diluted with DCM (70 mL) and washed with NH₄Cl solution (3×30 mL), NaHCO₃ solution (3×40 mL), water (50 mL) and brine (2×50 mL). Organic layer was separated, dried over anhydrous Na₂SO₄, filtered and the filtrate was evaporated to dryness. Solid residue thus obtained, was again evaporated with DCM (20 mL) and kept for drying overnight at 22° C. to afford 2S (6.97 g, 80% yield) as yellow solid. ¹H NMR (600 MHz, DMSO-d₆) δ 7.88-7.82 (m, 6H), 7.74 (dt, J=17.0, 5.7 Hz, 4H), 7.01 (s, 1H), 5.21 (d, J=3.4 Hz, 3H), 4.96 (dd, J=11.3, 3.4 Hz, 3H), 4.47 (d, J=8.5 Hz, 3H), 4.03-4.00 (m, 8H), 3.87 (dt, J=11.2, 8.9 Hz, 3H), 3.70 (dt, J=9.8, 5.8 Hz, 3H), 3.53 (dd, J=12.5, 6.2 Hz, 13H), 3.43-3.37 (m, 2H), 3.01 (dt, J=14.9, 7.4 Hz, 14H), 2.27 (t, J=6.4 Hz, 6H), 2.10 (s, 8H), 2.03 (q, J=6.9 Hz, 9H), 1.99 (s, 10H), 1.89 (s, 8H), 1.77 (s, 9H), 1.52-1.32 (m, 30H), 1.21 (s, 12H) ppm. ¹³C NMR (151 MHz, DMSO-d6) δ 172.53, 171.96, 171.93, 170.12, 170.07, 169.98, 169.70, 169.39, 162.35, 101.01, 70.48, 69.82, 68.70, 68.23, 67.34, 66.70, 61.46, 60.68, 59.48, 49.34, 38.36, 38.29, 36.38, 36.29, 36.01, 35.83, 35.45, 35.06, 32.54, 30.80, 29.38, 29.28, 29.02, 28.89, 28.84, 28.72, 28.67, 28.61, 26.37, 25.40, 25.36, 25.29, 22.80, 21.87, 20.57, 20.52, 20.50 ppm. MALDI mass calc. for C₉₇H₁₆₁N₁₁O₃₉Na [M+Na]⁺ 2128.38, found 2130.96.

[(3R,6R)-5-acetamido-6-[5-[3-[3-[3-[3-[3-[5-[(2R,5R)-3-acetamido-4,5-diacetoxy-6-(acetoxymethyltetrahydropyran-2-yl]oxypentanoylamino]propylamino]-3-oxo-propoxy]-2-[[3-[3-[5-[(2R,5R)-3-acetamido-4,5-diacetoxy-6-(acetoxymethyl)tetrahydropyran-2-yl]oxypentanoyl amino]propylamino]-3-oxo-propoxy]methyl]-2-[[12-oxo-12-(6-oxohexylamino)dodecanoyl]amino]propoxy]propanoylamino]propylamino]-5-oxo-pentoxy]-3,4-diacetoxy-tetrahydropyran-2-yl]methyl acetate (3S):

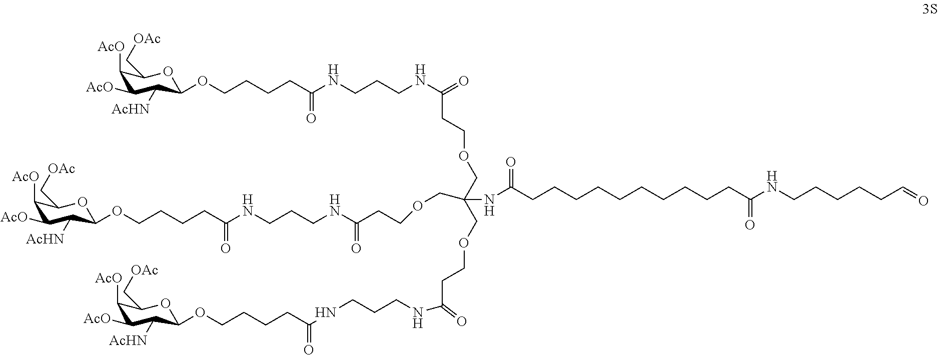

To a solution of 2S (1.0 g, 474.98 mol) in dry dichloromethane (50 mL) at 0° C., Dess-Martin periodinane (302.19 mg, 712.47 mol) was added slowly and then stirred for 5 hr maintaining 0° C. Reaction mixture was diluted with DCM (30 mL) and washed with 10% NaHCO₃ solution (50 mL) followed by 10% Na₂S₂O₃ solution (2×30 mL). Organic layer was separated, dried over anhydrous Na₂SO₄, filtered and the filtrate was evaporated to dryness to afford 3S (0.79 g, 79% yield) as white foam. ¹H NMR (600 MHz, DMSO-d₆) δ 9.65 (q, J=2.0 Hz, 1H), 7.88-7.81 (m, 6H), 7.75 (dt, J=11.4, 5.7 Hz, 4H), 7.01 (s, 1H), 5.21 (d, J=3.4 Hz, 3H), 4.96 (dd, J=11.3, 3.4 Hz, 3H), 4.47 (d, J=8.5 Hz, 3H), 4.03-4.00 (m, 8H), 3.87 (dt, J=11.2, 8.9 Hz, 3H), 3.70 (dt, J=9.6, 5.8 Hz, 3H), 3.53 (dd, J=12.5, 6.2 Hz, 14H), 3.40 (dt, J=9.8, 6.3 Hz, 3H), 3.01 (dp, J=13.1, 6.8 Hz, 15H), 2.40 (td, J=7.3, 1.7 Hz, 2H), 2.27 (t, J=6.4 Hz, 7H), 2.10 (s, 8H), 2.04 (t, J=7.3 Hz, 8H), 1.99 (s, 9H), 1.89 (s, 9H), 1.77 (s, 9H), 1.54-1.40 (m, 21H), 1.21 (s, 15H) ppm. ¹³C NMR (151 MHz, DMSO-d₆) δ 203.54, 172.53, 171.96, 170.12, 170.07, 169.98, 169.70, 169.40, 101.01, 70.48, 69.82, 68.70, 68.24, 67.35, 66.70, 61.46, 59.48, 54.97, 49.35, 42.99, 38.29, 38.16, 36.39, 36.30, 36.02, 35.91, 35.83, 35.46, 35.06, 29.38, 29.02, 28.90, 28.84, 28.73, 28.67, 28.61, 25.98, 25.39, 25.36, 22.80, 21.87, 21.28, 20.57, 20.52, 20.50 ppm. MALDI mass calc. for C₉₇H₁₅₉N₁₁O₃₉Na [M+Na]⁺ 2126.37, found 2128.87.

### 4-(2,2,2-trifluoro-N-((2-isobutyramido-4-oxo-3,4-dihydropteridin-6-yl)methyl)acetamido) benzoic acid (5S)

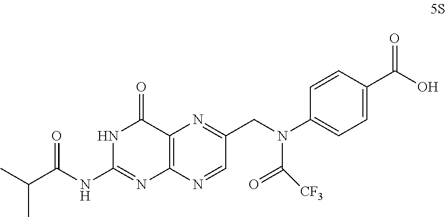 [SMILES: CC(C)C(=O)Nc1nc2ncc(CN(C(=O)C(F)(F)F)c3ccc(C(=O)O)cc3)nc2c(=O)[nH]1]

To a suspension of commercially available compound 4S⁴ (25 g, 61.2 mmol) and DMAP (11.25 g, 92 mmol) in anhydrous pyridine (400 mL), TBDPS chloride (42 g, 153 mmol) was added. The reaction mixture was stirred at room temperature for 30 h after which isobutyric anhydride (14.6 g, 92 mmol) was added and the mixture was slightly warmed. An additional 60 mL of pyridine was also added, and the reaction mixture was stirred at room temperature overnight. The reaction mixture became homogenous after which pyridine and other volatiles were concentrated in a rotary evaporator. The residue was stirred with EtOAc (1 μL) and acetic acid (100 mL) and water (500 mL) for 24 h. The thus obtained slurry was filtered, the residue was washed with water (500 mL), EtOAc (1 μL) and dried to obtain the pure product 5S as a white solid (26.1 g, 89%). ¹H NMR (DMSO-d₆, 400 MHz) δ=8.87 (s, 1H), 7.95 (d, J=8.6 Hz, 2H), 7.67 (d, J=8.6 Hz, 2H), 5.21 (s, 2H), 2.79-2.74 (m, 1H), 1.12 (d, J=6.83 Hz, 6H),¹³C NMR (DMSO-d₆) δ=180.72, 166.49, 159.25, 149.87, 147.68, 142. 69, 136.34, 134.45, 130.54, 129.16, 128.86, 127.49, 34.96, 33.09, 26.52, 18.88, 18.74. ¹⁹FNMR (DMSO-d₆) δ=−64.32.

### 5-(tert-butyl) 1-methyl (4-(2,2,2-trifluoro-N-((2-isobutyramido-4-oxo-3,4-dihydropteridin-6-yl)methyl)acetamido)benzoyl)-L-glutamate (6S)

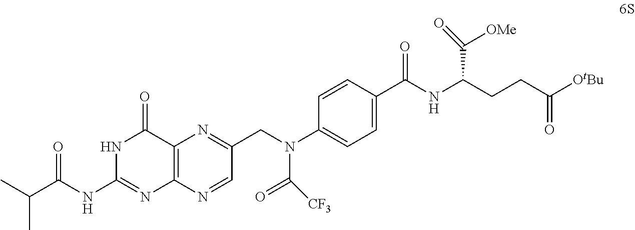 [SMILES: COC(=O)[C@H](CCC(=O)OC(C)(C)C)NC(=O)c1ccc(N(Cc2cnc3nc(NC(=O)C(C)C)[nH]c(=O)c3n2)C(=O)C(F)(F)F)cc1]

Compound 5S (2.4 g, 5 mmol) was dissolved in anhydrous DMF (20 mL), HBTU (1.9 g, 1 eq.) followed by DIEA (1 mL, 5 eq.) were added and stirred for 20 minutes. To this reaction mixture the 5-(tert-butyl)-1-methyl-L-glutamate hydrochloride (1.2 g, 1 eq) was added as a solution in DMF (6 mL). Reaction was monitored by TLC (8% MeOH/DCM, PMA stain). TLC of the reaction mixture showed completion of the reaction. The reaction mixture was slowly poured in ice with vigorous stirring. The precipitated product was filtered to get the product 6S as a white solid (Yield=2.85 g, 86%). ¹H NMR (DMSO-d₆,400 MHz) δ=12.33 (s, 1H), 11.94 (s, 1H), 8.88 (s, 1H), 8.82 (d, J=7.3 Hz, 1H), 7.90 (d, J=8.6 Hz, 2H), 7.68 (d, J=8.4 Hz, 2H), 5.22 (s, 2H), 4.46-4.40 (m, 1H), 3.62 (s, 3H), 2.86-2.73 (m, 1H), 2.32 (t, J=7.4 Hz, 2H) 2.05-1.90 (m, 2H), 1.35 (m, 9H), 1.12 (d, J=6.8 Hz, 6H).¹³C NMR DMSO-d₆) δ=180.75, 172.13, 171.45, 165.64, 159.10, 154.80, 149.97, 149.79, 147.72, 141.75, 134.15, 130.53, 128.70, 128.49, 117.50, 114.64, 79.79, 51.96, 51.91, 34.96, 31.22, 27.68, 25.71, 18.72.

### (S)-5-methoxy-5-oxo-4-(4-(2,2,2-trifluoro-N-((2-isobutyramido-4-oxo-3,4-dihydropteridin-6-yl)methyl)acetamido)benzamido)pentanoic acid (7S)²

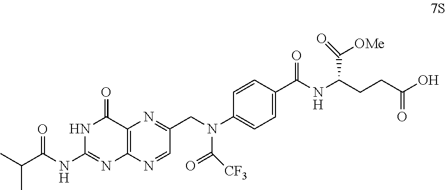 [SMILES: COC(=O)[C@H](CCC(=O)O)NC(=O)c1ccc(N(Cc2cnc3nc(NC(=O)C(C)C)[nH]c(=O)c3n2)C(=O)C(F)(F)F)cc1]

Compound 6S (2 g, 2.9 mmol) was dissolved in 20 mL of 50% TFA in dichloromethane and the solution was stirred at room temperature for 30 min. after which the TLC showed the complete disappearance of the starting ester. The reaction mixture was concentrated, and the residue was crystallized from DCM: hexanes (2:3) and crystallized product was filtered off and dried to obtain the pure product 7S (1.76 g, 96%) as off-white powder. H NMR (600 MHz, DMSO-d₆) δ 12.36 (s, 1H), 11.97 (s, 1H), 8.91 (s, 1H), 8.88 (d, J=7.3 Hz, 1H), 7.93 (d, J=8.3 Hz, 2H), 7.71 (d, J=8.2 Hz, 2H), 5.24 (s, 2H), 4.46 (ddd, J=9.8, 7.3, 5.1 Hz, 1H), 3.65 (s, 3H), 2.84-2.72 (m, 1H), 2.37 (t, J=7.4 Hz, 2H), 2.13-2.04 (m, 1H), 2.01-1.90 (m, 1H), 1.14 (d, J=6.9 Hz, 6H) ppm. ¹³C NMR (151 MHz, DMSO-d₆) δ 180.82, 173.76, 172.25, 165.76, 159.21, 156.11, 155.87, 155.64, 155.40, 154.81, 149.98, 149.88, 147.79, 141.83, 134.22, 130.58, 128.76, 128.54, 118.99, 117.08, 115.17, 113.25, 54.02, 52.12, 51.99, 35.02, 30.16, 25.72, 18.78 ppm. ¹⁹F NMR (565 MHz, DMSO-d₆) δ −66.06 ppm.

### Methyl-16-oxohexadecanoate⁵⁻⁶ was synthesized following the literature procedure

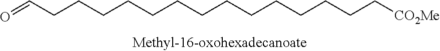 [SMILES: COC(=O)CCCCCCCCCCCCCCC=O]

To a clear solution of commercially available oxacyclohexadecan-2-one (1.03 g, 4.16 mmol) in methanol (10 mL) was added hydrogen chloride (151.68 mg, 4.16 mmol, 189.59 μL) dropwise at 22° C. and stirred for 12 hr. All the volatile matters were evaporated, and the residue was crystallized from chilled diethylether (30 mL) to afford methyl-16-hydroxylhexadecanoate⁷ (1.1 g, 3.84 mmol, 92% yield) as white solid which was used for next steps without further purification. ¹H NMR (400 MHz, CDCl₃) δ 3.64 (s, 3H), 3.61 (t, J=6.6 Hz, 2H), 2.28 (t, J=7.6 Hz, 2H), 1.99 (s, 1H), 1.65-1.49 (m, 3H), 1.24 (d, J=9.6 Hz, 23H) ppm. ¹³C NMR (101 MHz, CDCl₃) δ 174.46, 63.10, 51.52, 34.21, 32.89, 29.73, 29.71, 29.68, 29.67, 29.54, 29.36, 29.34, 29.24, 25.86, 25.05 ppm.

Dess-Martin Periodinane (2.02 g, 4.77 mmol) was dissolved in 20 mL of dichloromethane and cooled to 0° C. in an ice bath. A solution of methyl-16-hydroxylhexadecanoate (0.91 g, 3.18 mmol) in 20 mL of dichloromethane was added via a syringe to the mixture and the ice bath was removed. After stirring for 1 h at room temperature the reaction was quenched with a solution of NaHCO₃ (15 mL) and sodium thiosulfate (15 mL) and stirred for 5 min. After separation, the organic phase was washed with water (30 mL), brine (30 mL) successively and then dried over anhydrous Na₂SO₄. The solvent was removed, and the crude product was purified by column chromatography (gradient: 0-10% EtOAc in hexane) to yield methyl-16-oxohexadecanoate (0.77 g, 85% yield) as a white solid. Compound was stored at −20° C. ¹H NMR (500 MHz, CDCl₃) δ 9.74 (t, J=1.9 Hz, 1H), 3.64 (s, 3H), 2.40 (td, J=7.3, 1.9 Hz, 2H), 2.28 (t, J=7.6 Hz, 2H), 1.60 (pd, J=7.2, 5.1 Hz, 4H), 1.36-1.11 (m, 20H) ppm. ¹³C NMR (101 MHz, CDCl₃) δ 203.35, 174.75, 51.85, 44.35, 34.54, 30.03, 30.00, 29.87, 29.85, 29.78, 29.68, 29.60, 29.58, 25.39, 22.52 ppm.

### 2-[[(4R,6R)-6-(2,4-dioxopyrimidin-1-yl)-7-hydroxyl-2,5-dioxabicyclo[2.2.1]heptan-4-yl]methoxy]isoindoline-1,3-dione (10)

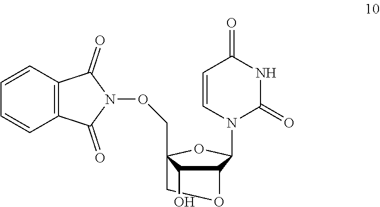 [SMILES: O=C1c2ccccc2C(=O)N1OC[C@]12CO[C@@H](C1O)[C@H](n1ccc(=O)[nH]c1=O)O2]

To a solution of commercially available compound 811-¹² (2.5 g, 9.76 mmol) in DMF (60 mL) was added triphenylphosphine (3.33 g, 12.68 mmol) and N-hydroxylphthalimide (2.07 g, 12.68 mmol). To this resulting mixture, DEAD (2.21 g, 12.68 mmol) was added dropwise at 0° C. The reaction mixture was stirred at 22° C. for 6 hr. The reaction mixture was then quenched with 10% aqueous NaHCO₃ (50 mL) and extracted with EtOAc (3×50 mL). Combined organic layer was dried over anhydrous Na₂SO₄, filtered and the filtrated was evaporated to dryness. The crude residue thus obtained was purified by flash column chromatography (gradient: 0-5% MeOH in DCM) to afford ELN0132-609 (3.01 g, 77% yield) as white solid. ¹H NMR (600 MHz, DMSO-d₆) δ 11.38 (s, 1H), 7.92-7.82 (m, 4H), 5.93 (d, J=4.4 Hz, 1H), 5.58 (dd, J=8.1, 1.3 Hz, 1H), 5.48 (s, 1H), 4.83 (d, J=11.9 Hz, 1H), 4.53 (d, J=11.9 Hz, 1H), 4.24 (s, 1H), 4.05-3.96 (m, 2H), 3.92 (d, J=8.0 Hz, 1H) ppm. ¹³C NMR (151 MHz, DMSO-d6) δ 163.25, 162.93, 162.90, 150.02, 139.54, 134.86, 134.83, 128.59, 123.35, 123.29, 100.95, 86.74, 86.22, 78.95, 73.89, 70.93, 69.57, 54.93 ppm. HRMS calc. for C₁₈H₁₆N₃O₈ [M+H]⁺ 402.0937, found 402.0922.

### N-[9-[(4R,6R)-4-[(1,3-dioxoisoindolin-2-yl)oxymethyl]-7-hydroxyl-2,5-dioxabicyclo[2.2.1] heptan-6-yl]purin-6-yl]benzamide (11)

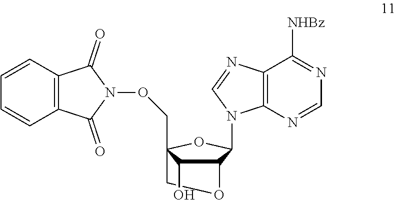

To a solution of commercially available 9¹³⁻¹⁴ (1.7 g, 4.43 mmol) in dimethylformamide (60 mL) was added triphenylphosphine (1.51 g, 5.76 mmol) and N-hydroxylphthalimide (940.42 mg, 5.76 mmol). To this resulting mixture, diethyl azodicarboxylate (1.06 g, 5.76 mmol, 1.11 mL) was added dropwise at 0° C. The reaction mixture was stirred at 22° C. for 6 hr. The reaction mixture was then quenched with 10% aqueous NaHCO₃ (50 mL) and extracted with EtOAc (3×50 mL). Combined organic layer was dried over anhydrous Na₂SO₄, filtered and the filtrated was evaporated to dryness. The crude residue thus obtained was purified by flash column chromatography (gradient: 0-5% MeOH in DCM) to afford 11 (1.45 g, 62% yield) as white solid. ¹H NMR (600 MHz, DMSO-d₆) δ 11.23 (s, 1H), 8.68 (s, 1H), 8.64 (s, 1H), 8.09-8.04 (m, 2H), 7.84-7.76 (m, 4H), 7.68-7.62 (m, 1H), 7.56 (t, J=7.8 Hz, 2H), 6.06 (s, 1H), 6.04 (d, J=4.5 Hz, 1H), 4.79 (d, J=12.3 Hz, 1H), 4.68 (s, 1H), 4.63-4.57 (m, 2H), 4.12 (d, J=8.0 Hz, 1H), 4.03 (d, J=8.0 Hz, 1H) ppm. ¹³C NMR (151 MHz, DMSO-d₆) δ 165.62, 162.87, 151.68, 151.52, 150.30, 141.93, 134.74, 133.34, 132.48, 128.52, 128.49, 128.44, 125.67, 123.21, 86.36, 85.19, 79.06, 73.47, 71.27, 70.92, 54.93 ppm. HRMS calc. for C₂₆H₂₁N₆O₇ [M+H]⁺ 529.1472, found 529.1450.

### 3-[(diisopropylamino)-[[(4R,6R)-4-[(1,3-dioxoisoindolin-2-yl)oxymethyl]-6-(2,4-dioxopyrimidin-1-yl)-2,5-dioxabicyclo[2.2.1]heptan-7-yl]oxy]phosphanyl]oxypropanenitrile (12)

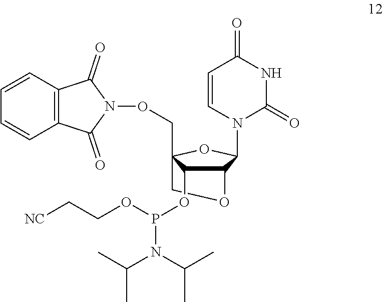 [SMILES: CC(C)N(C(C)C)P(OCCC#N)OC1[C@@H]2OC[C@]1(CON1C(=O)c3ccccc3C1=O)O[C@H]2n1ccc(=O)[nH]c1=O]

To a clear solution of 10 (2.0 g, 4.98 mmol) in DCM (30 mL) was added NMI (818.29 mg, 9.97 mmol, 794.45 μL) and DIPEA (3.22 g, 24.92 mmol, 4.34 mL) in single portions. After stirring the reaction mixture for 5 minutes at 22° C., 2-cyanoethyl-N,N-diisopropylchlorophosphoramidite (2.36 g, 9.97 mmol, 2.23 mL) was added and continued stirring for 1 hr and TLC was checked. Starting material was consumed and reaction mixture was diluted with DCM (15 mL). DCM layer was washed with 10% NaHCO₃ (2×25 mL) solution, and brine (30 mL). Organic layer was separated, dried over anhydrous Na₂SO₄, filtered and filtrate was evaporated at 36° C. to afford crude compound which was purified by flash chromatography (40-80% EtOAc in hexane) to afford 12 (2.45 g, 82% yield) as white hygroscopic foam. ¹H NMR (600 MHz, CD₃CN) δ 9.19 (s, 1H), 7.90-7.79 (m, 6H), 5.63 (dd, J=12.7, 8.2 Hz, 1H), 5.59 (d, J=4.2 Hz, 1H), 5.45 (s, 1H), 4.81-4.70 (m, 1H), 4.66-4.44 (m, 2H), 4.29 (dd, J=13.3, 7.4 Hz, 1H), 4.10-3.92 (m, 3H), 3.86-3.77 (m, 1H), 3.75-3.56 (m, 3H), 3.56-3.46 (m, 1H), 2.66-2.61 (m, 2H), 1.25-1.05 (m, 16H) ppm. ¹³C NMR (151 MHz, CD₃CN) δ 164.33, 164.27, 164.25, 164.16, 164.14, 151.06, 151.04, 140.41, 140.33, 135.83, 135.75, 130.00, 129.98, 129.95, 124.28, 124.25, 124.20, 124.18, 119.54, 119.49, 102.14, 102.11, 88.54, 88.46, 88.44, 88.41, 87.39, 87.36, 87.21, 87.17, 79.64, 79.63, 79.24, 79.22, 74.07, 72.68, 72.59, 72.42, 72.32, 72.23, 72.14, 60.95, 59.43, 59.34, 59.30, 59.26, 59.21, 59.15, 58.10, 55.32, 45.97, 45.93, 44.17, 44.13, 44.09, 44.04, 44.02, 24.81, 24.76, 24.74, 24.69, 23.16, 23.14, 23.09, 23.08, 21.95, 21.14, 21.00, 20.98, 20.93, 20.92 ppm. ³¹P NMR (243 MHz, CD₃CN) δ 148.98, 148.59 ppm. HRMS calc. for C₂₇H₃₃N₅O₉P [M+H]⁺ 602.2016, found 602.2022.

### N-[9-[(4R,6R)-7-[2-cyanoethoxy-(diisopropylamino)phosphanyl]oxy-4-[(1,3-dioxoisoindolin-2-yl)oxymethyl]-2,5-dioxabicyclo[2.2.1]heptan-6-yl]purin-6-yl]benzamide (13)

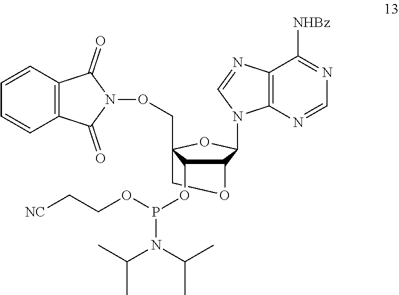

To a clear solution of 11 (1.0 g, 1.89 mmol) in DCM (25 mL) was added NMI (233.03 mg, 2.84 mmol, 226.24 μL) and DIPEA (1.22 g, 9.46 mmol, 1.65 mL) in single portions. After stirring the reaction mixture for 5 minutes at 22° C., 2-cyanoethyl-N,N-diisopropylchlorophosphoramidite (895.71 mg, 3.78 mmol, 845.01 μL) was added and continued stirring for 1 hr and TLC was checked. Starting material was consumed and reaction mixture was diluted with DCM (15 mL). DCM layer was washed with 10% NaHCO₃ (2×25 mL) solution, and brine (30 mL). Organic layer was separated, dried over anhydrous Na₂SO₄, filtered and filtrate was evaporated at 36° C. to afford crude compound which was purified by flash chromatography (40-80% EtOAc in hexane) to afford 13 (1.11 g, 80% yield) as white foam. ¹H NMR (600 MHz, CD₃CN) δ 9.34 (s, 1H), 8.59 (d, J=16.5 Hz, 1H), 8.47-8.31 (m, 1H), 8.02 (d, J=7.5 Hz, 2H), 7.80-7.70 (m, 4H), 7.65 (t, J=7.4 Hz, 1H), 7.55 (t, J=7.6 Hz, 2H), 6.09 (d, J=6.4 Hz, 1H), 4.93-4.74 (m, 3H), 4.72-4.59 (m, 1H), 4.19-4.07 (m, 2H), 3.87-3.67 (m, 2H), 3.66-3.50 (m, 2H), 2.60 (td, J=5.9, 1.2 Hz, 2H), 1.16-1.06 (m, 17H) ppm. ¹³C NMR (151 MHz, CD₃CN) δ 164.28, 164.20, 152.90, 142.25, 142.18, 135.72, 135.70, 133.58, 129.87, 129.78, 129.66, 129.16, 125.69, 125.67, 124.18, 124.15, 119.48, 119.45, 87.60, 87.55, 87.52, 87.50, 87.25, 87.13, 79.68, 79.66, 79.45, 79.42, 73.87, 73.58, 73.52, 73.48, 73.38, 73.11, 73.09, 73.04, 59.68, 59.55, 59.54, 59.41, 55.33, 49.48, 45.97, 45.93, 44.20, 44.12, 27.22, 24.83, 24.78, 24.76, 24.73, 24.68, 23.17, 23.15, 23.09, 23.08, 20.96, 20.95, 20.91, 20.90 ppm. HRMS calc. for C₃₅H₃₈N₈O₈P [M+H]⁺ 729.2550, found 729.2559.

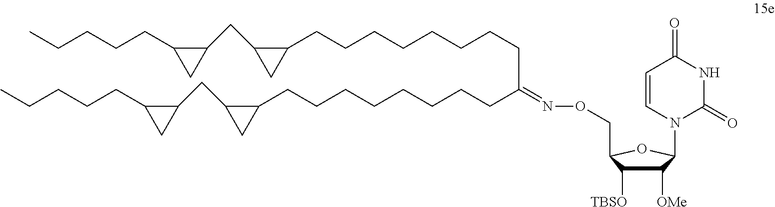

1-[(2R,5R)-4-[tert-butyl(dimethyl)silyl]oxy-3-methoxy-5-[[[9-[2-[(2-pentylcyclopropyl)methyl]cyclopropyl]-1-[8-[2-[(2-pentylcyclopropyl)methyl]cyclopropyl]octyl] nonylidenelamino]oxymethyl]tetrahydrofuran-2-yl]pyrimidine-2,4-dione (15e): To a solution of 14 (0.69 g, 1.78 mmol) in dichloromethane (20 mL), 1,17-bis[2-[(2-pentylcyclopropyl)methyl]cyclopropyl]heptadecan-9-one (2.08 g, 3.56 mmol) was added. To the resulting mixture, glacial acetic acid (4 mL) was added in single portion and stirred for 4 hr at 25° C. Reaction mixture was diluted with DCM (20 mL) and DI water (30 mL) was added. Organic layer was separated, dried over anhydrous Na₂SO₄, filtered and filtrate was evaporated to dryness. Crude compound was purified by flash column chromatography (gradient: 0-40% EtOAc in hexane) to afford (1.4 g, 83% yield) 15e as transparent gum. ¹H NMR (400 MHz, CDCl₃) δ 9.67-9.40 (m, OH), 7.75 (d, J=8.1 Hz, 1H), 5.90 (d, J=2.4 Hz, 1H), 5.64 (dd, J=8.1, 2.0 Hz, 1H), 4.42 (dd, J=12.3, 2.3 Hz, 1H), 4.27-4.12 (m, 3H), 3.62 (dd, J=4.8, 2.4 Hz, 1H), 3.54 (s, 3H), 2.41-2.32 (m, 2H), 2.30-2.21 (m, 2H), 2.19-2.11 (m, 2H), 1.53-1.24 (m, 54H), 1.20-0.96 (m, 5H), 0.89 (d, J=8.1 Hz, 14H), 0.82-0.72 (m, 5H), 0.72-0.63 (m, 6H), 0.60 (td, J=8.3, 4.1 Hz, 5H), 0.09 (d, J=3.7 Hz, 6H), -0.29 (ddd, J=11.1, 9.5, 5.1 Hz, 6H) ppm. ¹³C NMR (101 MHz, CDCl₃) δ 163.60, 162.43, 150.28, 139.96, 137.95, 129.14, 128.33, 125.41, 101.99, 88.18, 84.04, 83.14, 77.36, 71.24, 69.61, 58.56, 42.95, 34.05, 32.03, 30.33, 30.32, 30.14, 30.01, 29.75, 29.73, 29.65, 29.62, 29.58, 29.57, 29.53, 29.42, 29.04, 29.02, 28.88, 28.85, 28.39, 28.17, 28.02, 26.77, 26.09, 25.83, 24.05, 22.85, 21.57, 18.26, 16.19, 16.07, 16.05, 16.02, 15.80, 15.77, 15.76, 14.24, 11.17, 10.99, −4.59, −4.75 ppm. HRMS calc. for C₅₇H₁₀₂N₃O₆Si [M+H]⁺ 952.7538, found 952.7557.

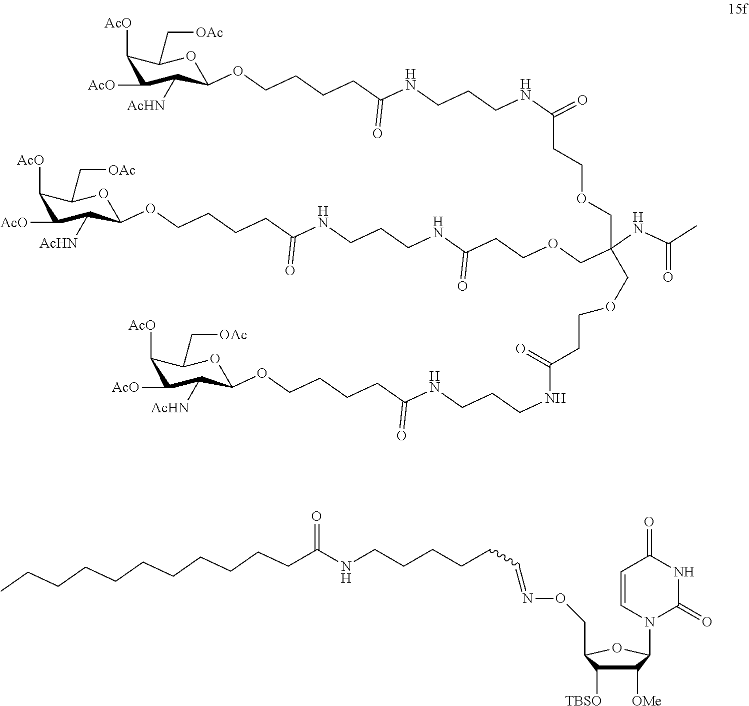

[(3R,6R)-5-acetamido-6-[5-[3-[3-[3-[3-[3-[5-[(2R,5R)-3-acetamido-4,5-diacetoxy-6-(acetoxy methyl)tetrahydropyran-2-yl]oxypentanoylamino]propylamino]-3-oxo-propoxy]-2-[[3-[3-[5-[(2R,5R)-3-acetamido-4,5-diacetoxy-6-(acetoxymethyl)tetrahydropyran-2-yl]oxypentanoylamino]propylamino]-3-oxo-propoxy]methyl]-2-[[12-[[(6Z)-6-[[(2R,5R)-3-[tert-butyl(dimethyl)silyl]oxy-5-(2,4-dioxopyrimidin-1-yl)-4-methoxy-tetrahydrofuran-2-yl]methoxyimino]hexyl]amino]-12-oxo-dodecanoyl]amino]propoxy]propanoylamino]propylamino]-5-oxo-pentoxy]-3,4-diacetoxy-tetrahydropyran-2-yl]methyl acetate (15f): To a clear solution of 14 (90.00 mg, 232.26 mol) in dichloromethane (20 mL), was added DIPEA (30.02 mg, 232.26 mol, 40.45 μL) at 22° C. The resulting mixture was stirred for 5 minutes and then to this reaction mixture, added aldehyde 3S (488.52 mg, 232.26 mol) in a single portion. The reaction mixture was stirred for 18 h and TLC was checked which showed consumption of starting materials. All the volatile matters were evaporated to dryness and the gummy residue obtained, was purified by flash column chromatography (gradient: 0-10% MeOH in DCM) to afford 15f (0.41 g, 71% yield) as yellowish white foam. ¹H NMR (600 MHz, DMSO-d₆) δ 11.42 (dd, J=7.3, 2.2 Hz, 1H), 7.88-7.82 (m, 6H), 7.78-7.72 (m, 3H), 7.70 (d, J=8.1 Hz, OH), 7.65 (d, J=8.1 Hz, OH), 7.49 (t, J=6.0 Hz, OH), 7.01 (s, 1H), 6.81 (t, J=5.3 Hz, OH), 5.79 (dd, J=7.9, 4.4 Hz, 1H), 5.71-5.56 (m, 1H), 5.21 (d, J=3.4 Hz, 3H), 4.96 (dd, J=11.3, 3.4 Hz, 3H), 4.47 (d, J=8.5 Hz, 3H), 4.31-4.24 (m, 1H), 4.19 (dd, J=12.3, 4.3 Hz, 1H), 4.15-4.07 (m, 3H), 4.05-3.96 (m, 10H), 3.92-3.83 (m, 4H), 3.70 (dt, J=9.5, 5.8 Hz, 3H), 3.53 (dd, J=12.6, 6.1 Hz, 13H), 3.40 (dt, J=9.7, 6.3 Hz, 2H), 3.34 (s, 2H), 3.16 (d, J=5.2 Hz, 6H), 3.02 (h, J=7.0 Hz, 14H), 2.27 (t, J=6.4 Hz, 7H), 2.10 (s, 9H), 2.03 (q, J=7.4 Hz, 9H), 1.99 (s, 9H), 1.89 (s, 9H), 1.77 (s, 8H), 1.53-1.37 (m, 29H), 1.29-1.18 (m, 16H), 0.87 (d, J=1.3 Hz, 11H), 0.08 (q, J=3.4 Hz, 6H) ppm. ¹³C NMR (151 MHz, DMSO-d6) δ 172.54, 171.98, 171.96, 170.13, 170.07, 169.98, 169.70, 169.41, 163.12, 152.58, 152.01, 150.49, 150.43, 140.34, 140.16, 102.08, 101.92, 101.02, 86.86, 86.70, 82.37, 82.16, 81.48, 81.33, 72.03, 71.83, 70.49, 69.86, 69.83, 69.69, 68.70, 68.24, 67.35, 66.70, 61.46, 59.49, 57.72, 57.62, 49.35, 48.64, 36.39, 36.30, 36.03, 35.92, 35.48, 35.07, 29.38, 29.05, 28.92, 28.88, 28.83, 28.77, 28.70, 28.61, 26.19, 25.97, 25.67, 25.64, 25.63, 25.44, 25.41, 25.37, 25.24, 22.81, 21.88, 20.57, 20.52, 20.50, 17.83, 17.82, −4.80, −4.84, −5.01, −5.10 ppm. MALDI calc. for C₁₁₃H₁₈₆N₁₄O₄₄SiNa [M+Na]+2495.86; found 2498.76.

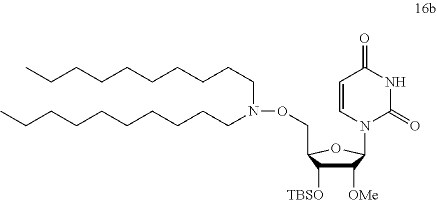

1-[(2R,5R)-4-[tert-butyl(dimethyl)silyl]oxy-5-[(didecylamino)oxymethyl]-3-methoxy-tetrahydrofuran-2-yl]pyrimidine-2,4-dione (16b): To a solution of 15b (550.00 mg, 1.05 mmol) in acetic acid (3 mL) was added sodium cyanoborohydride (184 mg, 2.93 mmol) under 15° C. The reaction mixture was stirred for 1 hr at 15° C. and decanal (500.41 mg, 3.14 mmol, 349.94 μL) in dichloromethane (2 mL) was added. The stirring was continued for 30 min and additional amount of sodium cyanoborohydride (184 mg, 2.93 mmol) was added. The resulting mixture was stirred for another 2 hr and then diluted with DCM and washed with ice water. The organic layer was separated and concentrated. The crude material was purified by flash column chromatography (0-50% EtOAc in hexane) to afford 16b (0.52 g, 74% yield). ¹H NMR (500 MHz, CDCl₃) δ 9.21 (d, J=7.4 Hz, 1H), 8.01 (d, J=8.1 Hz, 1H), 5.91 (d, J=2.3 Hz, 1H), 5.67 (dd, J=8.2, 1.5 Hz, 1H), 4.16 (dd, J=7.0, 4.9 Hz, 1H), 4.10-4.04 (m, 2H), 3.92-3.83 (m, 1H), 3.63 (d, J=6.6 Hz, 2H), 3.60 (dd, J=4.9, 2.4 Hz, 1H), 3.52 (s, 3H), 2.67 (td, J=7.0, 2.3 Hz, 4H), 1.55 (dt, J=14.5, 7.0 Hz, 6H), 1.34-1.22 (m, 43H), 0.90 (s, 9H), 0.87 (t, J=6.8 Hz, 10H), 0.09 (d, J=6.6 Hz, 6H) ppm. ¹³C NMR (101 MHz, CDCl₃) δ 163.50, 150.26, 140.36, 101.72, 87.90, 84.06, 82.88, 77.48, 77.16, 76.84, 71.19, 69.64, 63.21, 59.30, 58.29, 32.96, 32.04, 29.75, 29.71, 29.69, 29.58, 29.45, 27.63, 27.25, 25.89, 25.83, 22.81, 18.25, 14.23, −4.39, −4.74 ppm. HRMS calc. for C₃₆H₇₀N₃O₆Si [M+H]⁺ 668.5034, found 668.5040.

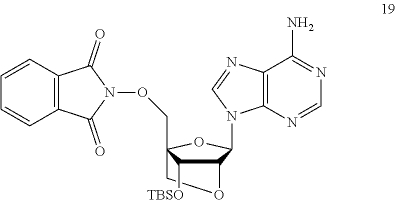 [SMILES: CC(C)(C)[Si](C)(C)OC1[C@@H]2OC[C@]1(CON1C(=O)c3ccccc3C1=O)O[C@H]2n1cnc2c(N)ncnc21]

2-[[(4R,6R)-6-(6-aminopurin-9-yl)-7-[tert-butyl(dimethyl)silyl]oxy-2,5-dioxabicyclo[2.2.1]heptan-4-yl]methoxy]isoindoline-1,3-dione (19): Commercially available compound 8S (1.0 g, 2.54 mmol) was converted to compound 19 (1.12 g, 82% yield) following the synthetic procedure mentioned for compound 11. ¹H NMR (600 MHz, CDCl₃) δ 8.11 (dd, J=3.6, 1.3 Hz, 1H), 7.97 (s, 1H), 7.63-7.54 (m, 4H), 6.38-6.11 (m, 1H), 5.84 (d, J=1.8 Hz, 1H), 4.66 (dd, J=9.3, 2.2 Hz, 2H), 4.48 (dd, J=11.9, 1.8 Hz, 1H), 4.39 (dd, J=11.9, 1.9 Hz, 1H), 4.03 (dd, J=7.8, 2.7 Hz, 1H), 3.93 (dd, J=7.7, 2.5 Hz, 1H), 0.74 (d, J=3.5 Hz, 9H), −0.00 (d, J=2.3 Hz, 3H), −0.03 (d, J=2.3 Hz, 3H) ppm. ¹³C NMR (151 MHz, CDCl₃) δ 163.08, 155.87, 155.85, 155.83, 153.14, 149.04, 149.02, 138.83, 138.81, 134.65, 128.75, 123.64, 119.96, 86.85, 86.42, 79.31, 72.74, 72.37, 72.27, 60.47, 25.65, 17.95, −4.66, −5.04 ppm. HRMS calc. for C₂₅H₃₁N₆O₆Si [M+H]⁺ 539.2074, found 539.2067.

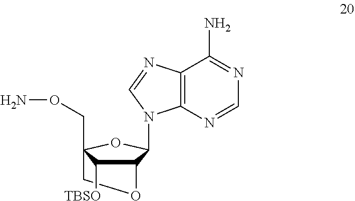 [SMILES: CC(C)(C)[Si](C)(C)OC1[C@@H]2OC[C@]1(CON)O[C@H]2n1cnc2c(N)ncnc21]

O-[[(4R,6R)-6-(6-aminopurin-9-yl)-7-[tert-butyl(dimethyl)silyl]oxy-2,5-dioxabicyclo[2.2.]heptan-4-yl]methyl]hydroxylamine (20): To a solution of 19 (1.0 g, 1.86 mmol) in dichloromethane (10 mL) at 0° C., N-methylhydrazine (83.47 mg, 1.86 mmol, 95.39 μL) was added in single portion and stirred for 1 hr. All volatile matters were evaporated to dryness and the crude product was purified by flash column chromatography (gradient: 0-10% MeOH in DCM) to afford 20 (0.61 g, 80% yield) as white solid. ¹H NMR (400 MHz, DMSO-d₆) δ 8.21 (s, 1H), 8.14 (s, 1H), 7.34 (s, 2H), 6.21 (s, 2H), 5.92 (s, 1H), 4.70 (s, 1H), 4.55 (s, 1H), 4.08-3.96 (m, 2H), 3.92-3.78 (m, 2H), 0.86 (s, 9H), 0.09 (d, J=2.5 Hz, 6H) ppm. ¹³C NMR (101 MHz, DMSO-d6) δ 156.07, 152.50, 148.49, 139.05, 118.97, 86.37, 85.47, 78.84, 72.74, 71.90, 71.48, 25.51, 17.61, -4.93, −5.13 ppm. HRMS calc. for C₁₇H₂₉N₆O₄Si [M+H]⁺ 409.2020, found 409.2033.

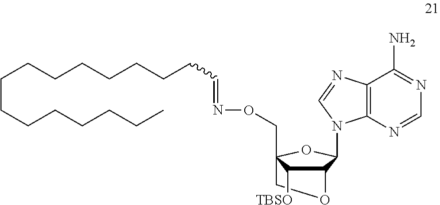 [SMILES: CCCCCCCCCCCCCCCC=NOC[C@]12CO[C@@H](C1O[Si](C)(C)C(C)(C)C)[C@H](n1cnc3c(N)ncnc31)O2]

9-[(4R,6R)-7-[tert-butyl(dimethyl)silyl]oxy-4-[(hexadecylideneamino)oxymethyl]-2,5-dioxabicyclo[2.2.1]heptan-6-yl]purin-6-amine (21): To a solution of 20 (0.3 g, 734.35 mol) in dichloromethane (10 mL), DIPEA (191.73 mg, 1.47 mmol, 258.40 μL) was added at 25° C. To this reaction mixture, hexadecanal (264.83 mg, 1.10 mmol) was added in single portion and resulting clear solution was stirred for 16 hr. The reaction mixture was diluted with DCM (20 mL), washed with water (10 mL), brine (20×2 mL) and organic layer was separated. DCM layer was dried over anhydrous Na₂SO₄, filtered and the filtrate was evaporated to dryness. The crude compound was purified by flash column chromatography (gradient: 10-60% EtOAc in hexane) to afford 21 (0.4 g, 633.98 mol, 86% yield). ¹H NMR (400 MHz, DMSO-d₆) δ 8.16 (d, J=11.3 Hz, 1H), 8.13 (d, J=1.6 Hz, 1H), 7.43 (t, J=6.0 Hz, 0.5H), 7.37-7.31 (m, 2H), 6.76 (t, J=5.6 Hz, 0.5H), 5.93 (d, J=1.5 Hz, 1H), 4.71 (d, J=6.8 Hz, 1H), 4.58 (d, J=1.6 Hz, 1H), 4.48-4.18 (m, 2H), 3.98 (dd, J=8.0, 1.3 Hz, 1H), 3.80 (dd, J=7.9, 3.3 Hz, 1H), 2.24-2.05 (m, 2H), 1.38 (dt, J=14.3, 7.3 Hz, 2H), 1.21 (t, J=6.1 Hz, 26H), 0.88-0.79 (m, 12H), 0.08 (dd, J=2.8, 1.5 Hz, 6H) ppm. ¹³C NMR (126 MHz, DMSO-d₆) δ 156.07, 152.50, 152.48, 152.18, 151.64, 148.44, 148.39, 138.80, 138.64, 119.05, 118.98, 86.48, 86.31, 85.62, 85.54, 78.85, 72.63, 72.39, 71.84, 71.69, 69.09, 68.89, 31.26, 29.00, 28.97, 28.92, 28.85, 28.83, 28.67, 28.60, 28.55, 28.35, 25.75, 25.45, 25.35, 25.08, 22.06, 17.58, 13.89, −4.95, −4.98, −5.21, −5.26 ppm. HRMS calc. for C₃₃H₅₉N₆O₄Si [M+H]⁺ 631.4367, found 631.4390.

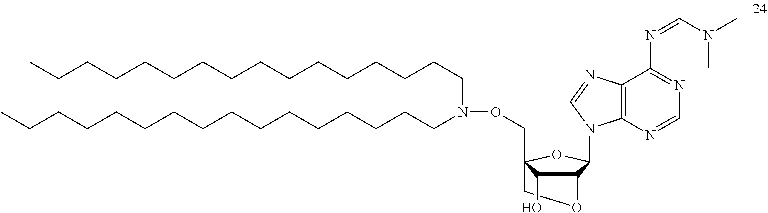 [SMILES: CCCCCCCCCCCCCCCCN(CCCCCCCCCCCCCCCC)OC[C@]12CO[C@@H](C1O)[C@H](n1cnc3c(/N=C\N(C)C)ncnc31)O2]

N′-[9-[(4R,6R)-4-[(dihexadecylamino)oxymethyl]-7-hydroxyl-2,5-dioxabicyclo[2.211 heptan-6-yl]purin-6-yl]-N,N-dimethyl-formamidine (24): To a clear solution of 23 (0.43 g, 471.26 mol) in THF (10 mL) was added TBAF (160.18 mg, 612.63 mol) in single portion and stirred for 4 hr at 22° C. All the volatile matters were evaporated under high vacuum pump and the crude residue thus obtained, was purified by flash column chromatography (gradient: 0-5% MeOH in DCM) to afford 24 (0.32 g, 85% yield) as white hygroscopic solid. ¹H NMR (600 MHz, CDCl₃) δ 8.93 (s, 1H), 8.49 (s, 1H), 8.08 (s, 1H), 6.06 (s, 1H), 4.64 (s, 1H), 4.40 (d, J=3.1 Hz, 1H), 4.18-4.13 (m, 3H), 4.01 (d, J=8.1 Hz, 1H), 3.87 (d, J=4.5 Hz, 1H), 3.26 (s, 3H), 3.21 (s, 3H), 2.71 (hept, J=6.4 Hz, 4H), 1.56 (p, J=7.4 Hz, 4H), 1.36-1.20 (m, 53H), 0.88 (t, J=7.0 Hz, 6H) ppm. ¹³C NMR (151 MHz, CDCl₃) δ 159.54, 158.17, 152.92, 150.63, 139.15, 126.46, 86.81, 86.39, 79.65, 72.24, 71.94, 68.82, 58.94, 41.58, 35.45, 32.07, 29.86, 29.84, 29.81, 29.79, 29.72, 29.51, 27.65, 27.08, 22.84, 14.27 ppm. HRMS calc. for C₄₆H₈₄N₇O₄ [M+H]⁺ 798.6585, found 798.6596.

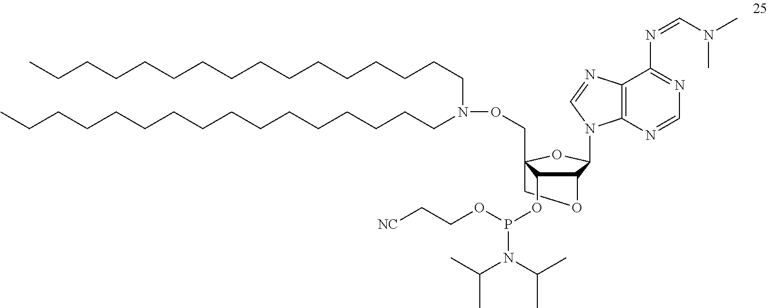 [SMILES: CCCCCCCCCCCCCCCCN(CCCCCCCCCCCCCCCC)OC[C@]12CO[C@@H](C1OP(OCCC#N)N(C(C)C)C(C)C)[C@H](n1cnc3c(/N=C\N(C)C)ncnc31)O2]

N′-[9-[(4R,6R)-7-[2-cyanoethoxy-(diisopropylamino)phosphanyl]oxy-4-[(dihexadecylamino)oxy methyl]-2,5-dioxabicyclo[2.21]heptan-6-yl]purin-6-yl]-N,N-dimethyl-formamidine (25): To a clear solution of 24 (0.289 g, 362.07 mol) in DCM (10 mL) was added NMI (44.59 mg, 543.10 mol, 43.29 μL) and DIPEA (233.97 mg, 1.81 mmol, 315.32 μL) in single portions. After stirring the reaction mixture for 5 minutes at 22° C., 2-cyanoethyl-N,N-diisopropylchlorophosphoramidite (171.39 mg, 724.14 mol, 161.69 μL) was added and continued stirring for 1 hr and TLC was checked. Starting material was consumed and reaction mixture was diluted with DCM (15 mL). DCM layer was washed with 10% NaHCO₃ (2×25 mL) solution, and brine (30 mL). Organic layer was separated, dried over anhydrous Na₂SO₄, filtered and filtrate was evaporated at 36° C. to afford crude compound which was purified by flash column chromatography (60-90% EtOAc in hexane) to afford 25 (0.3 g, 83% yield) as transparent gum. ¹H NMR (600 MHz, CD₃CN) δ 8.93 (s, 1H), 8.42 (d, J=4.5 Hz, 1H), 8.09 (d, J=7.0 Hz, 1H), 6.35 (s, OH), 6.00 (d, J=4.8 Hz, 1H), 4.79-4.54 (m, 1H), 4.48-4.32 (m, 1H), 4.13-4.00 (m, 6H), 3.83-3.70 (m, 3H), 3.63-3.43 (m, 4H), 3.19 (s, 4H), 2.76 (t, J=6.0 Hz, 2H), 2.73-2.62 (m, 5H), 1.61-1.50 (m, 5H), 1.39-1.09 (m, 81H), 1.07-0.98 (m, 7H), 0.88 (t, J=7.0 Hz, 6H) ppm. ¹³C NMR (151 MHz, CD₃CN) 6171.45, 160.45, 158.98, 158.96, 153.20, 151.55, 151.48, 139.74, 139.71, 127.12, 127.10, 119.23, 118.91, 118.66, 87.33, 87.29, 87.26, 87.22, 87.14, 73.31, 73.15, 73.07, 72.48, 72.40, 69.72, 69.58, 60.81, 59.77, 59.70, 59.57, 59.44, 59.38, 59.32, 59.25, 59.01, 58.98, 55.10, 45.85, 45.81, 43.97, 43.89, 43.87, 43.86, 43.79, 43.77, 41.53, 35.20, 32.53, 30.29, 30.27, 30.25, 30.21, 30.19, 30.12, 30.11, 29.97, 28.03, 28.00, 27.74, 27.69, 24.85, 24.82, 24.80, 24.77, 24.72, 24.60, 24.55, 23.28, 23.13, 23.11, 23.07, 23.06, 21.13, 20.93, 20.90, 20.85, 20.80, 20.76, 20.51, 20.46, 14.47, 14.39 ppm. ³¹P NMR (243 MHz, CD₃CN) δ 148.55, 148.40 ppm. HRMS calc. for C₅₅H₁₀₁N₉O₅P [M+H]⁺ 998.7663, found 998.7681.

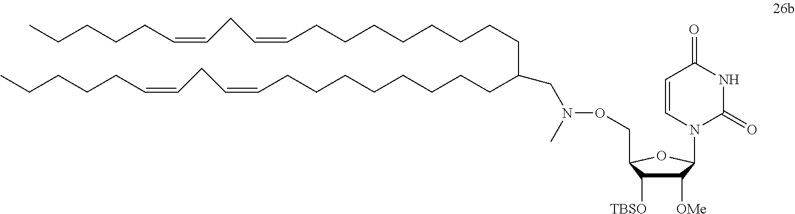

1-[(2R,5R)-4-[tert-butyl(dimethyl)silyl]oxy-3-methoxy-5-[[methyl-[(11Z,14Z)-2-[(9Z,12Z)-octadeca-9,12-dienyl]icosa-11,14-dienyl]amino]oxymethyl]tetrahydrofuran-2-yl]pyrimidine-2,4-dione (26b): To a clear solution of 15d (0.7 g, 768.87 umol) in glacial acetic acid (4 mL) at 15° C. was added sodium cyanoborohydride (128.18 mg, 2.00 mmol) in single portion and stirred for 1 hr. To this resulting reaction mixture, formaldehyde, 37% in aq. soln., (69.27 mg, 2.31 mmol, 64.14 uL) in dichloromethane (1 mL) was added slowly and stirred for 0.5 hr. To this mixture was added sodium cyanoborohydride (128.18 mg, 2.00 mmol) and stirred for 2.5 hr at 15° C. Reaction mixture was diluted with DCM (10 mL) and washed with brine (30 mL). Organic layer separated, dried over anhydrous Na₂SO₄, filtered and filtrate was evaporated to dryness. The crude residue thus obtained, was purified by flash column chromatography (gradient: 10-40% EtOAc in hexane) to afford 26b (0.46 g, 65% yield) as transparent gum. ¹H NMR (400 MHz, CDCl₃) δ 9.07 (d, J=2.2 Hz, 1H), 7.99 (d, J=8.1 Hz, 1H), 5.90 (d, J=2.3 Hz, 1H), 5.68 (dd, J=8.1, 2.1 Hz, 1H), 5.44-5.27 (m, 8H), 4.16-4.03 (m, 3H), 3.85 (dd, J=10.9, 2.3 Hz, 1H), 3.60 (dd, J=4.6, 2.3 Hz, 1H), 3.53 (s, 3H), 2.77 (t, J=6.5 Hz, 4H), 2.58 (s, 3H), 2.51 (s, 2H), 2.05 (q, J=6.8 Hz, 8H), 1.52 (d, J=7.3 Hz, 1H), 1.40-1.22 (m, 30H), 0.90 (d, J=8.3 Hz, 15H), 0.09 (d, J=4.7 Hz, 6H) ppm. ¹³C NMR (101 MHz, CDCl₃) δ 163.34, 150.20, 140.35, 130.32, 130.29, 130.27, 129.16, 128.35, 128.11, 128.09, 128.08, 125.43, 101.78, 87.98, 84.10, 82.69, 69.82, 69.61, 66.06, 58.40, 46.08, 35.98, 32.34, 32.21, 31.67, 30.34, 29.85, 29.80, 29.75, 29.50, 27.39, 27.35, 26.70, 26.62, 25.84, 25.79, 22.72, 18.25, 14.21, −4.41, −4.71 ppm. HRMS calc. for C₅₅H₁₀₀N₃O₆Si [M+H]⁺ 926.7381, found 926.7360.

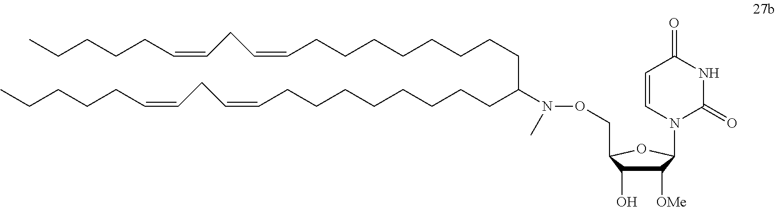

1-[(2R,5R)-4-hydroxyl-3-methoxy-5-[[methyl-[(11Z,14Z)-2-[(9Z,12Z)-octadeca-9,12-dienyl]icosa-11,14-dienyl]amino]oxymethyl]tetrahydrofuran-2-yl]pyrimidine-2,4-dione (27b): To a solution of 26b (0.42 g, 453.33 mol) in THF (10 mL) at 25° C., tetrabutylammonium fluoride, 1M in THF (119.73 mg, 453.33 μmol) was added slowly in single portion and then stirred for 12 hr. Volatile matters were removed in high vacuum pump and crude residue thus obtained was purified by flash column chromatography (gradient: 10-60% EtOAc in hexane) to afford 27b (0.3 g, 81% yield). ¹H NMR (400 MHz, CDCl₃) δ 8.99 (d, J=2.2 Hz, 1H), 7.95 (d, J=8.1 Hz, 1H), 5.95 (d, J=2.3 Hz, 1H), 5.70 (dd, J=8.1, 2.2 Hz, 1H), 5.44-5.27 (m, 8H), 4.18 (ddd, J=8.1, 7.0, 5.2 Hz, 1H), 4.11 (dd, J=11.0, 2.3 Hz, 1H), 4.05 (dt, J=7.1, 2.5 Hz, 1H), 3.90 (dd, J=11.1, 2.7 Hz, 1H), 3.74 (dd, J=5.2, 2.3 Hz, 1H), 3.61 (s, 3H), 2.77 (t, J=6.7 Hz, 4H), 2.67 (d, J=8.1 Hz, 1H), 2.60 (s, 3H), 2.52 (d, J=6.6 Hz, 2H), 2.05 (q, J=6.8 Hz, 8H), 1.53 (s, 2H), 1.43-1.23 (m, 39H), 0.95-0.84 (m, 6H) ppm. ¹³C NMR (101 MHz, CDCl₃) δ 163.21, 150.20, 140.11, 130.33, 130.30, 130.28, 128.10, 128.08, 128.07, 102.00, 87.39, 83.89, 83.06, 70.10, 68.78, 66.01, 58.80, 46.10, 35.95, 32.41, 32.23, 31.67, 30.32, 30.28, 29.84, 29.80, 29.75, 29.50, 29.48, 27.39, 27.34, 26.73, 26.63, 25.78, 22.72, 14.22 ppm. HRMS calc. for C₄₉H₈₆N₃O₆ [M+H]⁺ 812.6517, found 812.6537.

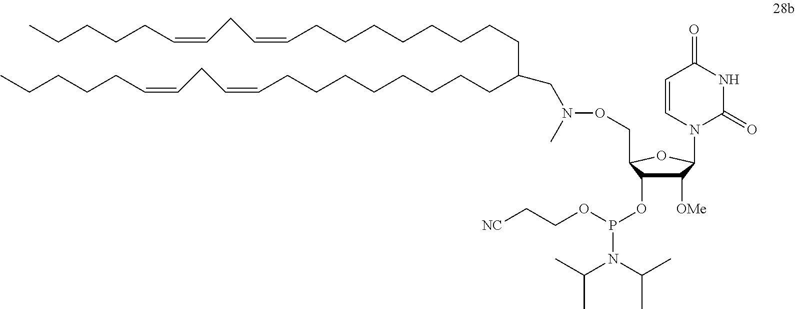

3-[(diisopropylamino)-[(2R,5R)-5-(2,4-dioxopyrimidin-1-yl)-4-methoxy-2-[[methyl-[(11Z,14Z)-2-[(9Z,12Z)-octadeca-9,12-dienyl]icosa-11,14-dienyl]amino]oxymethyl]tetrahydrofuran-3-yl]oxy-phosphanyl]oxypropanenitrile (28b): To a clear solution of 27b (0.27 g, 332.43 mol) in dichloromethane (10 mL), DIPEA (216.98 mg, 1.66 mmol, 292.43 μL) and NMI (96.49 mg, 1.16 mmol, 93.68 μL) were added at 25° C. To this reaction mixture, 2-cyanoethyl-N,N-diisopropylchlorophosphoramidite (165.64 mg, 664.85 μmol, 156.26 μL) was added slowly after 5 minutes and stirred for 1 hr. Reaction mixture was diluted with DCM (20 mL) and quenched with 10% NaHCO₃ solution (20 mL). Organic layer was separated, dried on anhydrous Na₂SO₄, filtered and filtrate was evaporated to dryness. The crude compound was thus obtained was purified by flash column chromatography (gradient: 10-50% EtOAc in hexane) to afford 28b (0.28 g, 83% yield). ¹H NMR (400 MHz, CDCl₃) δ 8.89 (s, 1H), 7.92 (dd, J=16.6, 8.2 Hz, 1H), 5.99 (dd, J=4.0, 2.1 Hz, 1H), 5.69 (dd, J=8.1, 2.0 Hz, 1H), 5.44-5.27 (m, 9H), 4.37-4.00 (m, 3H), 3.97-3.60 (m, 5H), 3.51 (d, J=9.7 Hz, 3H), 2.77 (t, J=6.5 Hz, 4H), 2.70-2.50 (m, 7H), 2.04 (dd, J=7.7, 6.0 Hz, 8H), 1.60-1.49 (m, 2H), 1.43-1.15 (m, 44H), 0.95-0.84 (m, 6H) ppm. ¹³C NMR (151 MHz, CDCl₃) δ 163.05, 163.01, 150.24, 150.20, 140.26, 140.14, 130.34, 130.30, 130.28, 130.26, 128.12, 128.10, 128.08, 128.06, 128.05, 117.80, 117.56, 102.15, 102.05, 87.60, 87.45, 83.53, 83.51, 83.02, 82.99, 82.42, 82.40, 82.32, 82.28, 70.80, 70.70, 70.49, 70.35, 70.22, 70.10, 65.99, 58.89, 58.82, 58.80, 58.77, 58.33, 58.31, 58.15, 58.03, 53.57, 46.12, 46.08, 43.51, 43.44, 43.43, 43.39, 43.36, 36.04, 36.01, 32.36, 32.34, 32.20, 32.17, 31.66, 30.39, 30.37, 30.35, 29.84, 29.81, 29.78, 29.76, 29.49, 27.38, 27.34, 26.76, 26.58, 25.77, 24.84, 24.79, 24.76, 24.74, 24.71, 22.71, 20.58, 20.56, 20.54, 20.52, 14.22 ppm. ³¹P NMR (162 MHz, CDCl₃) δ 148.57, 148.51 ppm. HRMS calc. for C₅₈H₁₀₃N₅O₇P [M+H]⁺ 1012.7595, found 1012.7560.

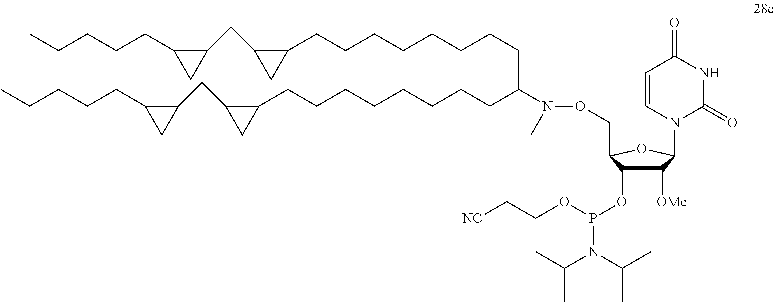

3-[(diisopropylamino)-[(2R,5R)-5-(2,4-dioxopyrimidin-1-yl)-4-methoxy-2-[[methyl-[9-[2-[(2-pentylcyclopropyl)methyl]cyclopropyl]-1-[8-[2-[(2-pentylcyclopropyl)methyl]cyclopropyl]octyl]nonyl]amino]oxymethyl]tetrahydrofuran-3-yl]oxy-phosphanyl]oxypropanenitrile (28c): To a clear solution of 27c (0.4 g, 468.22 mol) in dichloromethane (10 mL) was added NMI (57.66 mg, 702.34 mol, 55.98 μL) and diisopropylethylamine (302.57 mg, 2.34 mmol, 407.77 μL) in single portions. After stirring the reaction mixture for 5 minutes at 22° C., 2-cyanoethyl-N,N-diisopropylchlorophosphoramidite (221.64 mg, 936.45 mol, 209.09 μL) was added and continued stirring for 1 hr and TLC was checked. Starting material was consumed and reaction mixture was diluted with DCM (15 mL). DCM layer was washed with 10% NaHCO₃ (2×25 mL) solution, and brine (30 mL). Organic layer was separated, dried over anhydrous Na₂SO₄, filtered and filtrate was evaporated at 36° C. to afford crude compound which was purified by flash chromatography (60-100% EtOAc in hexane) to afford 28c (0.41 g, 83% yield) as transparent gum. ¹H NMR (600 MHz, CD₃CN) δ 9.05 (s, 1H), 7.84-7.77 (m, 1H), 5.89 (dd, J=13.3, 4.6 Hz, 1H), 5.60 (d, J=8.3 Hz, 1H), 5.43 (s, 3H), 4.39-4.09 (m, 2H), 4.05 (q, J=7.2 Hz, 1H), 3.95 (t, J=9.2 Hz, 1H), 3.86-3.69 (m, 3H), 3.64 (s, 2H), 3.48-3.32 (m, 3H), 2.65 (dt, J=13.2, 5.5 Hz, 2H), 2.55 (d, J=11.8 Hz, 3H), 1.55-1.08 (m, 76H), 1.02 (dt, J=15.0, 8.2 Hz, 1H), 0.87 (q, J=8.2 Hz, 6H), 0.77 (dt, J=14.1, 7.0 Hz, 4H), 0.68 (s, 6H), 0.58 (q, J=7.5 Hz, 5H), -0.21-0.41 (m, 4H) ppm. ¹³C NMR (151 MHz, CD3CN) δ 171.49, 163.68, 163.66, 151.18, 140.69, 140.68, 119.28, 119.19, 102.62, 102.59, 87.90, 87.50, 83.78, 83.27, 82.85, 71.79, 71.69, 71.40, 71.36, 71.32, 71.21, 67.01, 66.90, 60.83, 59.60, 59.48, 59.04, 58.97, 58.95, 58.91, 58.48, 58.46, 55.14, 43.96, 43.94, 43.88, 43.85, 40.42, 40.27, 32.53, 30.82, 30.54, 30.52, 30.48, 30.42, 30.23, 30.19, 30.17, 29.50, 29.33, 28.55, 28.45, 27.36, 27.33, 27.25, 25.02, 24.98, 24.93, 24.89, 24.85, 24.81, 23.33, 21.12, 20.96, 20.91, 20.86, 16.73, 16.62, 16.59, 16.28, 14.46, 14.43, 11.46, 11.44, 11.30 ppm. ³¹P NMR (243 MHz, CD₃CN) δ 150.00 ppm. HRMS calc. for C₆₁H₁₀₉N₅O₇P [M+H]⁺ 1054.8065, found 1054.8014.

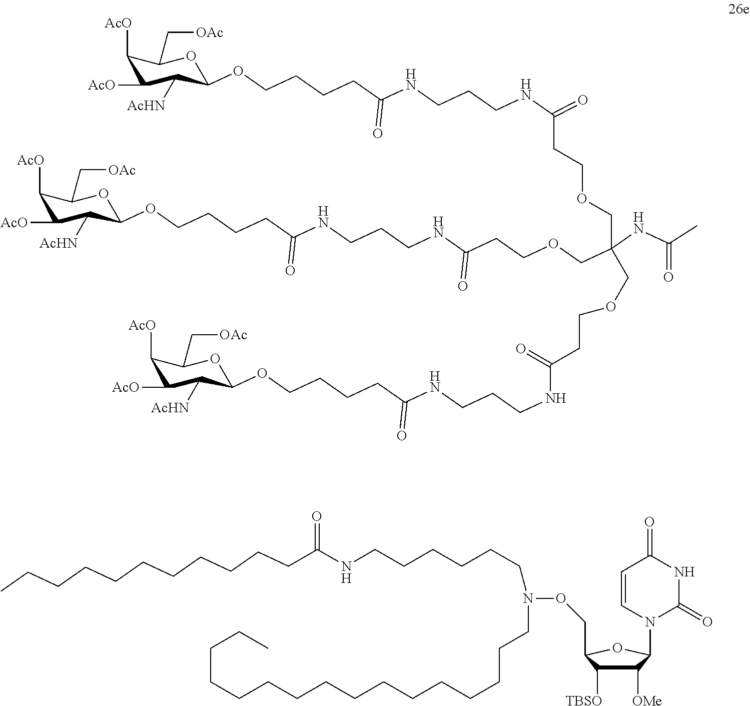

[(3R,6R)-5-acetamido-6-[5-[3-[3-[3-[3-[3-[5-[(2R,5R)-3-acetamido-4,5-diacetoxy-6-(acetoxymethyl)tetrahydropyran-2-yl]oxypentanoylamino]propylamino]-3-oxo-propoxy]-2-[[3-[3-[5-[(2R,5R)-3-acetamido-4,5-diacetoxy-6-(acetoxymethyl)tetrahydropyran-2-yl]oxypentanoyl amino]propylamino]-3-oxo-propoxy]methyl]-2-[[12-[6-[[(2R,5R)-3-ftert-butyl(dimethyl)silyl]oxy-5-(2,4-dioxopyrimidin-1-yl)-4-methoxy-tetrahydrofuran-2-yl]methoxy-hexadecyl-amino]hexylamino]-12-oxo-dodecanoyl]amino]propoxy]propanoylamino]propylamino]-5-oxo-pentoxy]-3,4-diacetoxy-tetrahydropyran-2-yl]methyl acetate (26e): To a clear solution of 15f (2.0 g, 808.79 mol) in dry dichloromethane (20 mL) and acetic acid (10 mL) was added sodium cyanoborohydride (132.14 mg, 2.10 mmol) in single portion and the reaction mixture was stirred for 1.5 hr at 15° C. To the resulting mixture was added palmitaldehyde (213.89 mg, 889.67 mol) and stirring was continued for 1 hr. Reaction was again cooled to 15° C. and sodium cyanoborohydride (132.14 mg, 2.10 mmol) was added. After 2.5 hr TLC showed consumption of starting materials. Reaction mixture was diluted with DCM (25 mL) and quenched with water (30 mL). Layers were separated and aqueous layer was washed with DCM (20 mL). Combined DCM layer was washed with brine (2×30 mL). Organic layer was separated, dried over anhydrous Na₂SO₄, filtered and the filtrate was evaporated to dryness. The residue thus obtained was purified by flash column chromatography (gradient: 2-20% MeOH in DCM) to afford 26e (1.52 g, 70% yield) as white foam. ¹H NMR (600 MHz, DMSO-d₆) δ 11.38 (d, J=2.2 Hz, 1H), 7.86-7.80 (m, 7H), 7.79-7.71 (m, 5H), 7.68 (t, J=5.6 Hz, 1H), 6.98 (s, 1H), 5.79 (d, J=3.9 Hz, 1H), 5.63 (dd, J=8.1, 2.2 Hz, 1H), 5.21 (d, J=3.4 Hz, 4H), 4.96 (dd, J=11.3, 3.4 Hz, 3H), 4.48 (d, J=8.5 Hz, 4H), 4.16 (t, J=5.5 Hz, 1H), 4.09 (q, J=5.2 Hz, 1H), 4.02 (h, J=4.3 Hz, 11H), 3.92 (td, J=5.1, 3.0 Hz, 1H), 3.90-3.82 (m, 5H), 3.78 (dd, J=11.0, 4.8 Hz, 1H), 3.70 (dt, J=9.7, 5.9 Hz, 4H), 3.53 (dd, J=12.1, 5.7 Hz, 15H), 3.40 (dt, J=9.8, 6.3 Hz, 4H), 3.35 (s, 3H), 3.17 (d, J=5.2 Hz, 2H), 3.02 (tt, J=13.0, 6.5 Hz, 17H), 2.65-2.59 (m, 5H), 2.27 (t, J=6.4 Hz, 8H), 2.10 (s, 9H), 2.04 (t, J=7.3 Hz, 9H), 1.99 (s, 9H), 1.89 (s, 9H), 1.77 (s, 11H), 1.53-1.40 (m, 29H), 1.31-1.19 (m, 40H), 0.86 (d, J=13.4 Hz, 12H), 0.08 (d, J=1.0 Hz, 6H) ppm. ¹³C NMR (151 MHz, DMSO-d6) δ 172.47, 171.92, 171.86, 170.07, 170.01, 169.92, 169.64, 169.35, 163.07, 150.34, 140.24, 101.63, 100.98, 95.40, 86.87, 82.10, 81.82, 72.23, 70.46, 69.86, 69.81, 68.66, 68.25, 67.32, 66.68, 61.42, 59.46, 58.47, 58.42, 57.50, 49.35, 48.60, 38.28, 36.36, 36.27, 36.00, 35.90, 35.45, 35.04, 31.30, 29.35, 29.14, 29.04, 29.01, 28.98, 28.95, 28.93, 28.91, 28.89, 28.85, 28.74, 28.71, 28.67, 28.59, 26.82, 26.62, 26.53, 26.49, 26.36, 25.58, 25.38, 25.33, 22.76, 22.10, 21.84, 20.52, 20.47, 20.45, 17.75, 13.96, −4.73, −5.15 ppm. MALDI calc. for C₁₂₉H₂₂₀N₁₄O₄₄SiNa [M+Na]⁺ 2722.31; found 2725.66.

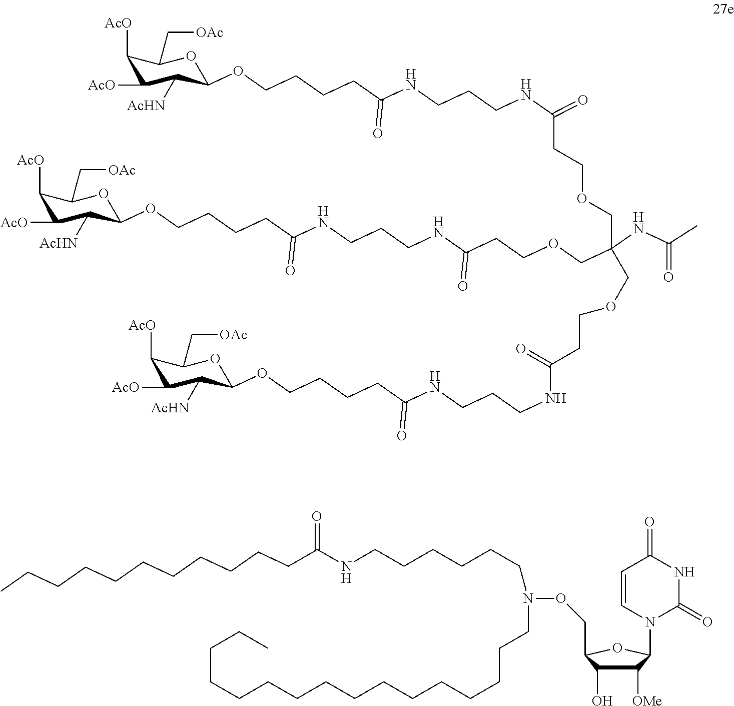

[(3R,6R)-5-acetamido-6-[5-[3-[3-[3-[3-[3-[5-[(2R,5R)-3-acetamido-4,5-diacetoxy-6-(acetoxymethyl)tetrahydropyran-2-yl]oxypentanoylamino]propylamino]-3-oxo-propoxy]-2-[[3-[3-[5-[(2R,5R)-3-acetamido-4,5-diacetoxy-6-(acetoxymethyl)tetrahydropyran-2-yl]oxypentanoyl amino]propylamino]-3-oxo-propoxy]methyl]-2-[[12-[6-[[(2R,5R)-5-(2,4-dioxopyrimidin-1-yl)-3-hydroxyl-4-methoxy-tetrahydrofuran-2-yl]methoxy-hexadecyl-amino]hexylamino]-12-oxo-dodecanoyl]amino]propoxy]propanoylamino]propylamino]-5-oxo-pentoxy]-3,4-diacetoxy-tetrahydropyran-2-yl]methyl acetate (27e): To a clear solution of 26e (1.5 g, 555.71 mol) in THF (25 mL) was added TBAF (188.88 mg, 722.42 mol) in single portion and stirred for 12 hr at 22° C. All the volatile matters were evaporated under high vacuum pump and the crude residue thus obtained, was purified by flash column chromatography (gradient: 5-20% MeOH in DCM) to afford 27e (1.21 g, 84% yield) as white foam. ¹H NMR (600 MHz, CD₃CN) δ 9.52 (s, 1H), 7.83 (d, J=8.2 Hz, 1H), 7.17 (t, J=6.0 Hz, 2H), 6.99 (t, J=5.9 Hz, 2H), 6.91 (d, J=9.4 Hz, 2H), 6.64 (d, J=6.5 Hz, 2H), 5.85 (d, J=3.7 Hz, 1H), 5.61 (d, J=8.2 Hz, 1H), 5.28 (dd, J=3.5, 1.1 Hz, 3H), 5.03 (dd, J=11.3, 3.4 Hz, 3H), 4.54 (d, J=8.5 Hz, 3H), 4.15 (t, J=5.3 Hz, 1H), 4.11 (dd, J=11.3, 6.8 Hz, 3H), 4.05 (dd, J=11.3, 6.1 Hz, 3H), 4.01-3.91 (m, 8H), 3.86-3.76 (m, 5H), 3.62 (d, J=5.0 Hz, 13H), 3.52-3.44 (m, 6H), 3.18 (dq, J=12.4, 6.5 Hz, 14H), 3.11 (q, J=6.6 Hz, 2H), 2.69-2.64 (m, 3H), 2.34 (t, J=5.9 Hz, 7H), 2.14 (td, J=7.3, 1.8 Hz, 6H), 2.10 (s, 11H), 1.98 (s, 9H), 1.91 (s, 9H), 1.85 (s, 10H), 1.60 (p, J=7.3 Hz, 13H), 1.55-1.48 (m, 13H), 1.44 (p, J=6.9 Hz, 1H), 1.38-1.24 (m, 33H) ppm. ¹³C NMR (151 MHz, CD₃CN) δ 174.47, 174.01, 173.99, 173.97, 173.94, 172.42, 172.40, 172.38, 171.36, 171.34, 171.32, 171.29, 171.19, 171.01, 164.09, 151.43, 141.21, 102.43, 102.21, 96.64, 88.07, 84.14, 83.82, 73.08, 71.65, 71.41, 70.13, 69.96, 69.89, 68.42, 67.91, 62.53, 62.46, 60.74, 59.88, 59.77, 58.85, 51.13, 39.71, 37.52, 37.48, 37.23, 37.12, 36.93, 36.52, 32.64, 30.45, 30.42, 30.40, 30.38, 30.36, 30.32, 30.29, 30.24, 30.20, 30.15, 30.12, 30.08, 30.00, 29.97, 29.84, 29.82, 29.80, 29.78, 29.53, 28.15, 27.82, 27.80, 27.70, 27.44, 26.63, 26.60, 24.27, 23.39, 23.18, 20.95, 20.91, 14.42, 13.77 ppm. MALDI calc. for C₁₂₃H₂₀₆N₁₄O₄₄Na [M+Na]⁺ 2608.05; found 2611.20.

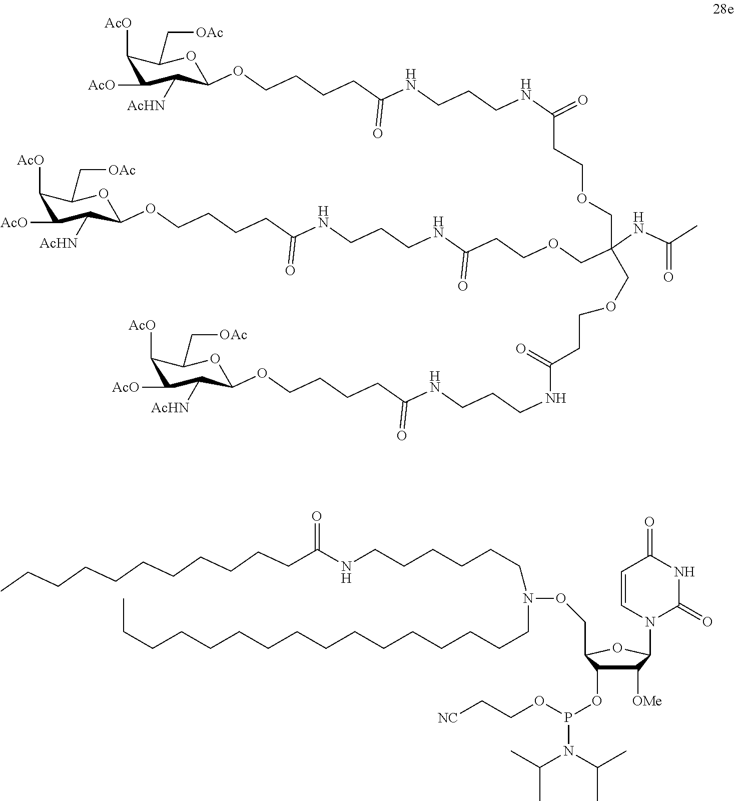

[(3R,6R)-5-acetamido-6-[5-[3-[3-[3-[3-[3-[5-[(2R,5R)-3-acetamido-4,5-diacetoxy-6-(acetoxymethyl)tetrahydropyran-2-yl]oxypentanoylamino]propylamino]-3-oxo-propoxy]-2-[[3-[3-[5-[(2R,5R)-3-acetamido-4,5-diacetoxy-6-(acetoxymethyl)tetrahydropyran-2-yl]oxypentanoylamino]propylamino]-3-oxo-propoxy]methyl]-2-[[12-[6-[[(2R,5R)-3-[2-cyanoethoxy-(diisopropylamino)phosphanyl]oxy-5-(2,4-dioxopyrimidin-1-yl)-4-methoxy-tetrahydrofuran-2-yl]methoxy-hexadecyl-amino]hexylamino]-12-oxo-dodecanoyl]amino]propoxy]propanoyl amino]propylamino]-5-oxo-pentoxy]-3,4-diacetoxy-tetrahydropyran-2-yl]methyl acetate (28e): To a clear solution of 27e (1.0 g, 386.85 mol) in DCM (20 mL) was added NMI (63.52 mg, 773.69 μmol, 61.67 μL) and DIPEA (249.98 mg, 1.93 mmol, 336.90 μL) in single portions. After stirring the reaction mixture for 5 minutes at 22° C., 2-cyanoethyl-N,N-diisopropylchlorophosphoramidite (183.12 mg, 773.69 μmol, 172.75 μL) was added and continued stirring for 1 hr and TLC was checked. Starting material was consumed and reaction mixture was diluted with DCM (15 mL). DCM layer was washed with 10% NaHCO₃ (25×2 mL) solution, and brine (30 mL). Organic layer was separated, dried over anhydrous Na₂SO₄, filtered and filtrate was evaporated at 36° C. to afford crude gummy compound which was triturated with 1:1 hexane and diethylether (20 mL). The solid residue was then dissolved in methyl-tert-butylether (30 mL) and the organic layer was washed with 30% DMF in water (20×2 mL) followed by brine (30×3 mL). Organic layer was separated, dried over anhydrous Na₂SO₄, filtered and the filtrated was evaporated to dryness. Residue was co-evaporated with diethyl ether and dried under high vacuum pump overnight to afford 28e (0.78 g, 72% yield) as white foam. ¹H NMR (600 MHz, CD₃CN) δ 9.40 (s, 1H), 7.88-7.76 (m, 1H), 7.19 (q, J=7.5 Hz, 3H), 7.05-6.88 (m, 7H), 6.61 (d, J=17.9 Hz, 1H), 5.88 (dd, J=13.4, 4.4 Hz, 1H), 5.61 (d, J=8.2 Hz, 1H), 5.28 (dd, J=3.4, 1.1 Hz, 4H), 5.03 (dd, J=11.2, 3.3 Hz, 4H), 4.55 (d, J=8.6 Hz, 4H), 4.41-4.23 (m, 1H), 4.14-4.02 (m, 8H), 4.02-3.93 (m, 8H), 3.90-3.77 (m, 4H), 3.70-3.60 (m, 16H), 3.52-3.44 (m, 5H), 3.18 (dd, J=11.7, 6.1 Hz, 12H), 2.72-2.63 (m, 8H), 2.37-2.32 (m, 7H), 2.18-2.05 (m, 19H), 1.98 (s, 11H), 1.91 (s, 10H), 1.85 (s, 9H), 1.64-1.50 (m, 21H), 1.36-1.17 (m, 79H), 1.13 (s, 19H), 0.88 (t, J=6.9 Hz, 3H) ppm. ¹³C NMR (151 MHz, CD₃CN) δ 174.42, 174.38, 173.97, 173.96, 173.76, 172.39, 172.38, 172.36, 171.33, 171.31, 171.29, 171.18, 171.01, 164.01, 163.25, 151.46, 141.07, 141.01, 119.58, 119.52, 102.60, 102.21, 88.33, 87.93, 83.62, 83.60, 83.35, 83.14, 82.99, 82.96, 73.11, 72.97, 72.85, 71.81, 71.67, 71.53, 71.42, 70.10, 69.95, 68.42, 67.92, 62.53, 60.74, 59.81, 59.74, 59.63, 59.30, 59.27, 59.17, 59.01, 59.00, 58.58, 58.56, 51.12, 49.48, 45.88, 45.83, 45.80, 45.74, 44.11, 44.10, 44.03, 44.01, 39.74, 37.53, 37.48, 37.21, 37.10, 36.94, 36.52, 32.64, 30.43, 30.40, 30.38, 30.36, 30.34, 30.31, 30.29, 30.26, 30.24, 30.21, 30.19, 30.15, 30.13, 30.08, 30.05, 30.02, 30.00, 29.93, 29.90, 29.82, 29.51, 28.19, 28.16, 27.91, 27.89, 27.86, 27.81, 27.57, 27.23, 26.66, 26.62, 25.08, 25.04, 24.99, 24.95, 24.94, 24.90, 24.83, 24.27, 23.39, 23.17, 21.04, 21.02, 20.97, 20.95, 20.90, 14.42 ppm. ³¹P NMR (243 MHz, CD₃CN) δ 149.91, 149.87 ppm. MALDI calc. for C₁₃₂H₂₂₃N₁₆O₄₅PNa [M+Na]+2808.26; found 2811.21.

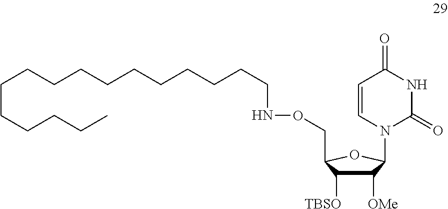

1-[(2R,5R)-4-[tert-butyl(dimethyl)silyl]oxy-5-[(hexadecylamino)oxymethyl]-3-methoxy-tetrahydrofuran-2-yl]pyrimidine-2,4-dione (29): To a solution of 15c (0.61 g, 1.00 mmol) in glacial acetic acid (10 mL), sodium cyanoborohydride (166.74 mg, 2.60 mmol, 98% purity) was added and stirred for 4 hr at 15° C. Reaction mixture was diluted with DCM (20 mL) and the organic layer was washed with water (20 mL). Organic layer was dried over anhydrous Na₂SO₄, filtered, and the filtrate was evaporated to dryness. The crude compound was purified by flash column chromatography to afford 29 (0.52 g, 85% yield) as transparent gum. ¹H NMR (500 MHz, CDCl₃) δ 9.50 (s, 1H), 7.88 (d, J=8.1 Hz, 1H), 5.88 (d, J=2.1 Hz, 1H), 5.70 (d, J=8.1 Hz, 1H), 4.17 (dd, J=7.5, 4.8 Hz, 1H), 4.14-4.05 (m, 2H), 3.90-3.83 (m, 1H), 3.63 (dd, J=4.8, 2.2 Hz, 1H), 3.54 (s, 3H), 2.95 (tt, J=9.4, 4.7 Hz, 2H), 1.53-1.46 (m, 2H), 1.26 (s, 26H), 0.91 (s, 10H), 0.88 (t, J=6.8 Hz, 3H), 0.10 (d, J=4.0 Hz, 6H) ppm. ¹³C NMR (101 MHz, CDCl₃) δ 163.75, 163.70, 150.30, 140.18, 101.98, 88.37, 84.01, 82.47, 77.48, 77.16, 76.84, 71.74, 69.57, 69.54, 58.56, 52.36, 32.05, 29.82, 29.79, 29.72, 29.71, 29.65, 29.48, 27.43, 27.27, 25.81, 22.81, 18.26, 14.24, −4.54, −4.79 ppm. HRMS calc. for C₃₂H₆₂N₃O₆Si [M+H]⁺ 612.4408, found 612.4423.

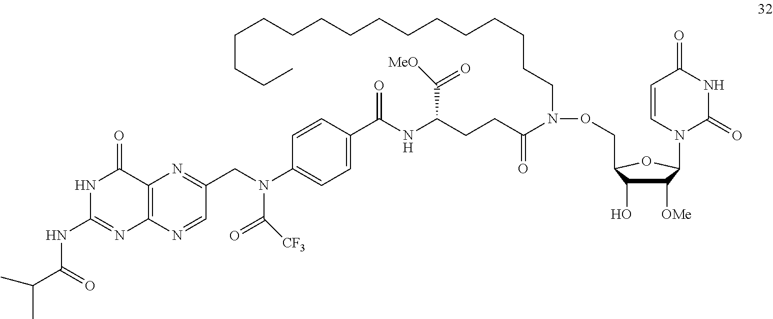

Methyl(2S)-5-[[5-(2,4-dioxopyrimidin-1-yl)-3-hydroxyl-4-methoxy-tetrahydrofuran-2-yl]methoxy-hexadecyl-amino]-2-[[4-[[2-(2-methylpropanoylamino)-4-oxo-3H-pteridin-6-yl]methyl-(2,2,2-trifluoroacetyl)amino]benzoyl]amino]-5-oxo-pentanoate (31): To a clear solution of 30 (0.9 g, 740.48 mol) in THF (20 mL) was added TBAF (251.69 mg, 962.62 mol, 278.72 μL) in single portion and stirred for 12 hr at 22° C. All the volatile matters were evaporated under high vacuum pump and the crude residue thus obtained, was purified by flash column chromatography (gradient: 5% MeOH in DCM) to afford 31 (0.55 g, 67% yield) as yellow solid. ¹H NMR (600 MHz, DMSO-d₆) δ 12.35 (s, 1H), 11.97 (s, 1H), 11.38 (s, 1H), 8.90 (s, 1H), 8.87 (d, J=7.3 Hz, 1H), 7.90 (d, J=8.1 Hz, 2H), 7.69 (d, J=8.1 Hz, 2H), 7.63 (s, 2H), 5.83 (d, J=4.8 Hz, 1H), 5.61 (dt, J=7.9, 1.6 Hz, 1H), 5.37 (d, J=6.1 Hz, 1H), 5.27-5.18 (m, 2H), 4.44 (q, J=7.2 Hz, 1H), 4.09 (t, J=11.1 Hz, 3H), 4.04-3.95 (m, 2H), 3.81 (t, J=5.0 Hz, 1H), 3.62 (s, 3H), 3.54 (qt, J=13.9, 7.2 Hz, 2H), 3.34 (dd, J=2.7, 1.3 Hz, 7H), 3.05-2.99 (m, 1H), 2.78 (hept, J=6.9 Hz, 1H), 2.59-2.51 (m, 3H), 2.10 (dt, J=14.2, 6.8 Hz, 1H), 1.96 (h, J=7.3 Hz, 1H), 1.57 (td, J=10.1, 6.1 Hz, 1H), 1.48 (hept, J=6.9 Hz, 2H), 1.31 (h, J=7.6 Hz, 1H), 1.24-1.11 (m, 42H), 0.91 (td, J=7.3, 1.3 Hz, 1H), 0.86-0.81 (m, 3H) ppm. ¹³C NMR (151 MHz, DMSO-d₆) δ 180.81, 172.76, 172.37, 165.68, 162.95, 159.14, 156.10, 155.87, 155.63, 155.40, 154.84, 150.42, 149.99, 149.84, 147.81, 141.84, 140.21, 134.20, 130.57, 129.06, 128.69, 128.50, 118.96, 117.05, 115.14, 113.23, 102.07, 86.62, 81.67, 81.12, 79.20, 73.12, 68.56, 57.61, 54.05, 52.34, 51.91, 51.78, 44.34, 35.01, 31.31, 29.06, 29.03, 28.97, 28.90, 28.82, 28.73, 28.67, 28.08, 26.30, 26.06, 25.36, 25.10, 22.11, 19.38, 18.77, 13.95, 13.53 ppm. ¹⁹F NMR (565 MHz, DMSO-d₆) δ −66.12 ppm. HRMS calc. for C₅₂H₇₂F₃N₁₀O₁₃ [M+H]⁺ 1101.5232, found 1101.5220.

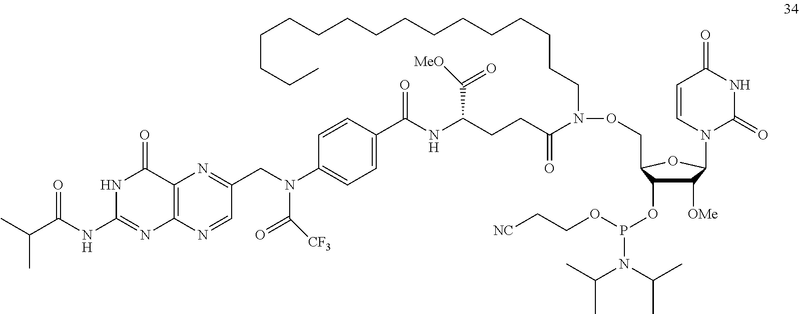

Methyl(2S)-5-[[3-[2-cyanoethoxy-(diisopropylamino)phosphanyl]oxy-5-(2,4-dioxopyrimidin-1-yl)-4-methoxy-tetrahydrofuran-2-yl]methoxy-hexadecyl-amino]-2-[[4-[[2-(2-methylpropanoylamino)-4-oxo-3H-pteridin-6-yl]methyl-(2,2,2-trifluoroacetyl)amino]benzoyl]amino]-5-oxo-pentanoate (32): To a clear solution of 31 (0.31 g, 281.52 mol) in dichloromethane (20 mL) was added NMI (46.23 mg, 563.04 mol, 44.88 μL) and DIPEA (181.92 mg, 1.41 mmol, 245.17 μL) in single portions. After stirring the reaction mixture for 5 minutes at 22° C., 2-cyanoethyl-N,N-diisopropylchlorophosphoramidite (133.26 mg, 563.04 mol, 125.72 μL) was added and continued stirring for 1 hr and TLC was checked. Starting material was consumed and reaction mixture was diluted with DCM (15 mL). DCM layer was washed with 10% NaHCO₃ (2×25 mL) solution, and brine (30 mL). Organic layer was separated, dried over anhydrous Na₂SO₄, filtered and filtrate was evaporated at 36° C. to afford crude compound which was triturated with 1:1 diethylether-hexane mixture (30 mL) to afford off-white precipitate. This residue was dissolved in DCM (20 mL), evaporated and co-evaporated with MTBE (20 mL) to afford 32 (0.33 g, 90% yield) as off-white foam. ¹H NMR (600 MHz, CD₃CN) δ 8.79 (d, J=1.6 Hz, 1H), 7.87-7.75 (m, 3H), 7.60-7.32 (m, 3H), 5.89-5.74 (m, 1H), 5.62-5.56 (m, 1H), 5.17 (s, 2H), 4.57-4.20 (m, 2H), 4.18-3.96 (m, 4H), 3.91-3.60 (m, 9H), 3.55-3.38 (m, 5H), 2.76-2.62 (m, 4H), 2.25-2.01 (m, 10H), 1.55 (s, 2H), 1.28-1.15 (m, 56H), 0.87 (t, J=7.0 Hz, 3H) ppm. ¹³C NMR (151 MHz, CD₃CN) δ 181.79, 173.27, 173.26, 173.24, 166.79, 166.72, 163.78, 163.73, 163.25, 160.69, 157.51, 157.27, 156.09, 151.23, 151.16, 150.99, 149.18, 143.20, 140.83, 138.91, 135.55, 132.09, 132.07, 129.99, 129.56, 129.31, 129.27, 119.67, 119.61, 116.42, 103.10, 73.41, 73.17, 73.11, 71.24, 71.13, 69.67, 59.66, 59.54, 59.16, 59.15, 59.13, 59.05, 59.02, 58.94, 58.60, 55.21, 54.01, 53.91, 52.85, 49.48, 45.99, 45.94, 44.13, 44.06, 44.04, 36.79, 36.53, 33.60, 32.63, 31.26, 30.40, 30.38, 30.35, 30.30, 30.28, 30.24, 30.22, 30.20, 30.07, 30.04, 30.02, 29.95, 29.60, 27.73, 27.38, 27.31, 27.22, 26.22, 25.02, 25.00, 24.97, 24.95, 24.89, 24.87, 24.83, 23.39, 23.17, 23.15, 23.09, 23.08, 21.05, 21.00, 20.96, 20.62, 20.57, 19.76, 19.17, 19.14, 14.39 ppm. ¹⁹F NMR (565 MHz, CD₃CN) δ −67.69, −67.70 ppm. ³¹P NMR (243 MHz, CD₃CN) δ 150.16, 149.92 ppm. HRMS calc. for C₆₁H₈₉F₃N₁₂O₁₄P [M+H]⁺ 1301.6311, found 1301.6333.

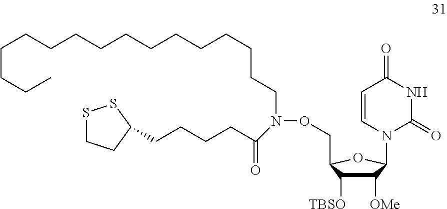

N-[[(2R,5R)-3-[tert-butyl(dimethyl)silyl]oxy-5-(2,4-dioxopyrimidin-1-yl)-4-methoxy-tetrahydrofuran-2-yl]methoxy]-5-(dithiolan-3-yl)-N-hexadecyl-pentanamide (31): To a clear solution of (±)-1,2-dithiolane-3-pentanoic acid (454.16 mg, 2.16 mmol) in DMF (10 mL), were added 1-hydroxyl-7-azabenzotriazole tetrahydrate (458.21 mg, 2.16 mmol), N-(3-dimethylaminopropyl)-N′-ethylcarbodiimide hydrochloride (435.28 mg, 2.16 mmol) and DIPEA (469.34 mg, 3.60 mmol, 632.53 μL) in single portions. After 5 minutes, 29 (1.1 g, 1.80 mmol) was added and the resulting mixture was stirred for 8 hr at 25° C. Reaction mixture was diluted with EtOAc (30 mL), and cold water (20 mL). Organic layer was separated, washed with NaHCO₃ solution (20 mL), water (20 mL) and brine (30×3 mL). EtOAc layer was separated, dried on anhydrous Na₂SO₄, filtered and the filtrate was evaporated to dryness. The crude mass obtained, was purified by flash column chromatography to afford 31(0.95 g, 66% yield). ¹H NMR (400 MHz, CDCl₃) δ 9.30 (s, 1H), 7.59 (s, 1H), 5.86 (t, J=1.8 Hz, 1H), 5.72 (d, J=8.1 Hz, 1H), 4.23-4.12 (m, 2H), 4.10-4.00 (m, 1H), 3.73-3.67 (m, 1H), 3.67-3.49 (m, 5H), 3.22-3.04 (m, 2H), 2.49-2.30 (m, 3H), 1.96-1.82 (m, 1H), 1.75-1.55 (m, 4H), 1.51-1.37 (m, 2H), 1.33-1.21 (m, 28H), 0.91 (s, 9H), 0.89-0.84 (m, 3H), 0.11 (d, J=6.1 Hz, 6H) ppm. ¹³C NMR (101 MHz, CDCl₃) δ 163.17, 150.04, 140.05, 102.51, 89.19, 83.27, 81.34, 72.22, 69.80, 58.37, 56.52, 56.50, 40.37, 38.63, 34.90, 34.87, 32.06, 29.83, 29.82, 29.79, 29.71, 29.49, 29.46, 29.17, 29.15, 26.91, 25.80, 24.48, 22.82, 18.24, 14.25, −4.27, −4.73 ppm. HRMS calc. for C₄₀H₇₄N₃O₇S₂Si [M+H]⁺ 800.4737, found 800.4751.

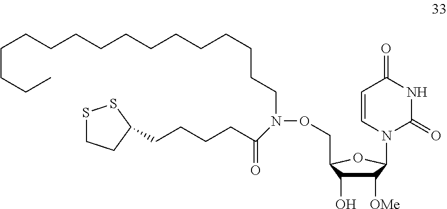

N-[[(2R,5R)-5-(2,4-dioxopyrimidin-1-yl)-3-hydroxyl-4-methoxy-tetrahydrofuran-2-yl]methoxy]-5-(dithiolan-3-yl)-N-hexadecyl-pentanamide (33): To a solution of 31 (0.85 g, 1.06 mmol) in THF (10 mL) at 25° C., tetrabutylammonium fluoride, 1M in THF (280.52 mg, 1.06 mmol, 1.06 mL) was added slowly in single portion and then stirred for 4 hr. Volatile matters were removed in high vacuum pump and crude residue thus obtained was purified by flash column chromatography (gradient: 10-60% EtOAc in hexane) to afford 33 (0.52 g, 71% yield). ¹H NMR (400 MHz, CDCl₃) δ 8.98 (s, 1H), 7.61 (s, 1H), 5.92 (s, 1H), 5.72 (dd, J=8.2, 1.8 Hz, 1H), 4.31 (dt, J=10.4, 2.0 Hz, 1H), 4.24 (s, 1H), 4.14-4.01 (m, 2H), 3.81 (dd, J=5.4, 1.7 Hz, 1H), 3.65 (s, 3H), 3.61-3.51 (m, 2H), 3.23-3.06 (m, 2H), 2.78 (s, 1H), 2.45 (tt, J=15.3, 7.7 Hz, 3H), 1.97-1.84 (m, 1H), 1.82-1.46 (m, 7H), 1.56-1.40 (m, 2H), 1.25 (s, 26H), 0.88 (t, J=6.8 Hz, 3H) ppm. ¹³C NMR (101 MHz, CDCl₃) δ 162.88, 149.97, 139.40, 102.55, 88.22, 83.31, 81.54, 71.95, 68.47, 58.97, 56.58, 56.55, 40.41, 38.62, 34.85, 34.79, 32.44, 32.07, 29.85, 29.80, 29.72, 29.51, 29.45, 29.16, 29.13, 26.89, 24.55, 22.84, 14.27 ppm. HRMS calc. for C₃₄H₆₀N₃O₇S₂ [M+H]⁺ 686.3873, found 686.3855.

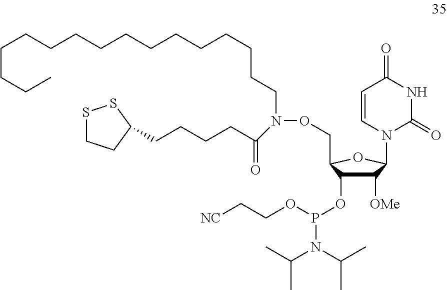

N-[[(2R,5R)-3-[2-cyanoethoxy-(diisopropylamino)phosphanyl]oxy-5-(2,4-dioxopyrimidin-1-yl)-4-methoxy-tetrahydrofuran-2-yl]methoxy]-5-(dithiolan-3-yl)-N-hexadecyl-pentanamide (35): To a clear solution of 33 (0.35 g, 510.22 mol) in dichloromethane (15 mL), DIPEA (333.04 mg, 2.55 mmol, 448.84 μL) and NMI (148.09 mg, 1.79 mmol, 143.78 μL) were added at 22° C. To this reaction mixture, 2-cyanoethyl-N,N-diisopropylchlorophosphoramidite (254.23 mg, 1.02 mmol, 239.84 μL) was added slowly after 5 minutes and stirred for 1 hr. Reaction mixture was diluted with DCM (10 mL) and quenched with 10% NaHCO₃ solution (20 mL). Organic layer was separated, dried on anhydrous Na₂SO₄, filtered and filtrate was evaporated to dryness. The crude compound was thus obtained was purified by flash column chromatography (gradient: 10-50% EtOAc in hexane) to afford 35 (0.205 g, 45% yield) as transparent yellow gum. ¹H NMR (400 MHz, CDCl₃) δ 8.65 (s, 1H), 7.58 (s, 1H), 5.99-5.86 (m, 1H), 5.78-5.65 (m, 1H), 4.44-4.31 (m, 2H), 4.27-4.18 (m, 2H), 4.13-3.98 (m, 1H), 3.96-3.85 (m, 2H), 3.83-3.50 (m, 10H), 3.23-3.06 (m, 2H), 2.65 (dt, J=12.5, 6.2 Hz, 2H), 2.52-2.34 (m, 3H), 1.97-1.84 (m, 1H), 1.78-1.56 (m, 2H), 1.53-1.39 (m, 2H), 1.36-1.16 (m, 43H), 0.92-0.83 (m, 3H) ppm. ¹³C NMR (101 MHz, CDCl₃) δ 162.96, 162.93, 150.14, 140.01, 117.79, 116.99, 102.67, 102.61, 82.95, 72.52, 70.30, 70.16, 60.50, 58.96, 58.47, 58.33, 58.27, 58.10, 57.92, 57.72, 56.58, 56.55, 45.49, 45.43, 43.60, 43.57, 43.47, 43.45, 40.39, 38.61, 34.91, 34.87, 32.04, 29.82, 29.78, 29.74, 29.51, 29.48, 29.19, 29.15, 26.94, 26.92, 24.87, 24.80, 24.74, 24.72, 24.69, 24.51, 23.12, 23.10, 23.03, 23.01, 22.81, 20.68, 20.61, 20.57, 20.27, 20.20, 14.32, 14.23 ppm. ³¹P NMR (162 MHz, CDCl₃) δ 150.77 ppm. HRMS calc. for C₄₃H₇₇N₅O₈PS₂ [M+H]⁺ 886.4951, found 886.4966.

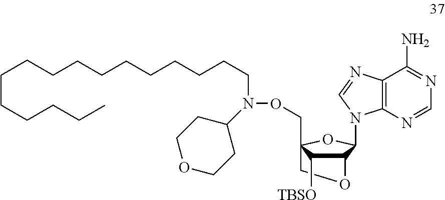 [SMILES: CCCCCCCCCCCCCCCCN(OC[C@]12CO[C@@H](C1O[Si](C)(C)C(C)(C)C)[C@H](n1cnc3c(N)ncnc31)O2)C1CCOCC1]

9-[(4R,6R)-7-[tert-butyl(dimethyl)silyl]oxy-4-[[hexadecyl(tetrahydropyran-4-yl)amino]oxy methyl]-2,5-dioxabicyclo[2.2.1]heptan-6-yl]purin-6-amine (37): To a clear solution of 20 (0.8 g, 1.96 mmol) in a mixture of acetic acid (10 mL) and dichloromethane (5 mL) was added tetrahydro-4H-pyran-4-one (196.06 mg, 1.96 mmol, 181.54 μL) and stirred for 3 hr at 22° C. To this reaction mixture was added sodium cyanoborohydride (319.95 mg, 5.09 mmol). Reaction mixture was stirred for 2 hr and then hexadecanal (706.21 mg, 2.94 mmol) was added in single portion at 15° C. After stirring the mixture for 1 hr, a second batch of sodium cyanoborohydride (319.95 mg, 5.09 mmol) was added and kept stirring for 9 hrs. Reaction mixture was diluted with DCM (20 mL), organic layer was washed with water (20 mL) and brine (2×30 mL). Organic layer was separated, dried over anhydrous Na₂SO₄, filtered and the filtrate was evaporated to dryness. The crude thus obtained was purified by flash column chromatography (gradient: 20-70% EtOAc in hexane) to afford 37 (0.7 g, 50% yield) as white solid. ¹H NMR (400 MHz, CDCl₃) δ 8.36 (s, 1H), 8.05 (s, 1H), 6.03 (s, 2H), 5.97 (d, J=0.7 Hz, 1H), 4.72 (s, 1H), 4.36 (s, 1H), 4.11-3.98 (m, 5H), 3.95 (d, J=7.7 Hz, 1H), 3.72 (s, 2H), 3.37 (td, J=11.9, 2.1 Hz, 2H), 2.87-2.68 (m, 3H), 1.81 (s, 3H), 1.73-1.55 (m, 4H), 1.25 (d, J=3.6 Hz, 24H), 0.88 (s, 12H), 0.06 (d, J=13.8 Hz, 6H) ppm. ¹³C NMR (101 MHz, CDCl₃) δ 158.55, 155.59, 153.07, 152.07, 148.89, 138.88, 132.82, 132.59, 129.70, 127.24, 125.24, 124.77, 120.08, 87.09, 86.85, 79.07, 72.67, 72.19, 70.23, 67.14, 67.10, 62.82, 55.11, 37.70, 32.07, 29.85, 29.82, 29.81, 29.77, 29.72, 29.51, 27.65, 27.05, 25.70, 22.84, 17.99, 14.28, 0.15, −4.45, −4.95 ppm. HRMS calc. for C₃₈H₆₉N₆O₅Si [M+H]⁺ 717.5099, found 717.5095.

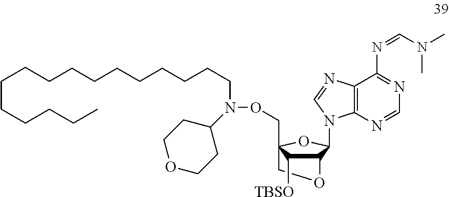 [SMILES: CCCCCCCCCCCCCCCCN(OC[C@]12CO[C@@H](C1O[Si](C)(C)C(C)(C)C)[C@H](n1cnc3c(/N=C\N(C)C)ncnc31)O2)C1CCOCC1]

N′-[9-[(4R,6R)-7-[tert-butyl(dimethyl)silyl]oxy-4-[[hexadecyl(tetrahydropyran-4-yl)amino]oxymethyl]-2,5-dioxabicyclo[2.2.1heptan-6-yl]purin-6-yl]-N,N-dimethyl-formamidine (39): To a clear solution of 37 (0.7 g, 976.20 gmol) in DMF (5 mL) was added dimethylformamide dimethyl acetal (116.32 mg, 976.20 mol, 130.70 μL) in single portion and the reaction mixture was stirred at 65° C. for 10 hr. TLC was checked, and volatile matters was removed under high vacuum pump. Residue was dissolved in DCM (50 mL) and the organic layer was washed with brine (3×30 mL). DCM layer was then dried over anhydrous Na₂SO₄, filtered and the filtrate was evaporated to dryness. Crude mass thus obtained, was purified by flash column chromatography (gradient: 0-5% MeOH in DCM) to afford 39 (0.64 g, 85% yield) as transparent gum. ¹H NMR (600 MHz, DMSO-d₆) δ 8.91 (s, 1H), 8.40 (s, 1H), 8.26 (s, 1H), 5.98 (s, 1H), 4.63 (s, 1H), 4.59 (s, 1H), 4.10 (d, J=11.0 Hz, 1H), 3.97-3.91 (m, 2H), 3.83 (dd, J=9.6, 5.9 Hz, 3H), 3.27-3.19 (m, 5H), 3.13 (s, 3H), 2.79-2.60 (m, 3H), 1.70 (d, J=15.3 Hz, 2H), 1.54-1.41 (m, 5H), 1.30-1.18 (m, 27H), 0.85 (d, J=6.1 Hz, 12H), 0.07 (s, 6H) ppm. ¹³C NMR (151 MHz, DMSO-d₆) δ 159.27, 157.97, 151.88, 150.44, 140.44, 125.59, 86.15, 85.52, 78.67, 72.18, 71.68, 70.46, 66.01, 61.94, 54.19, 40.68, 34.56, 31.30, 29.04, 29.00, 28.99, 28.94, 28.89, 28.71, 26.86, 26.35, 25.44, 22.10, 17.54, 13.96, −4.83, −5.32 ppm. HRMS calc. for C₄₁H₇₄N₇O₅Si [M+H]⁺ 772.5521, found 772.5514.

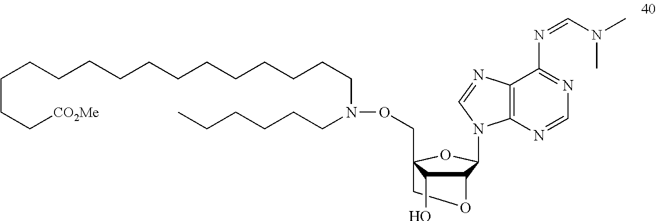 [SMILES: CCCCCCN(CCCCCCCCCCCCCCCC(=O)OC)OC[C@]12CO[C@@H](C1O)[C@H](n1cnc3c(/N=C\N(C)C)ncnc31)O2]

Methy-16-[[(4R,6R)-6-[6-[(Z)-dimethylaminomethyleneamino]purin-9-yl]-7-hydroxyl-2,5-dioxabicyclo[2.21]heptan-4-yl]methoxy-hexyl-amino]hexadecanoate (40): To a clear solution of 38 (0.37 g, 453.32 mol) in THF (25 mL) was added TBAF (154.08 mg, 589.32 mol, 170.63 μL) in single portion and stirred for 4 hr at 22° C. All the volatile matters were evaporated under high vacuum pump and the crude residue thus obtained, was purified by flash column chromatography (gradient: 0-5% MeOH in DCM) to afford 40 (0.28 g, 88% yield) as transparent gum. ¹H NMR (600 MHz, DMSO-d₆) δ 8.91 (s, 1H), 8.42 (s, 1H), 8.25 (s, 1H), 5.94 (s, 1H), 5.78 (d, J=4.4 Hz, 1H), 4.42 (s, 1H), 4.29 (d, J=4.4 Hz, 1H), 4.15 (d, J=11.2 Hz, 1H), 3.99 (d, J=11.1 Hz, 1H), 3.95 (d, J=7.8 Hz, 1H), 3.82 (d, J=7.8 Hz, 1H), 3.57 (s, 3H), 3.20 (s, 3H), 3.13 (s, 3H), 2.67-2.55 (m, 5H), 2.27 (t, J=7.4 Hz, 2H), 1.49 (pd, J=7.5, 2.8 Hz, 7H), 1.31-1.17 (m, 35H), 0.83 (t, J=6.8 Hz, 3H) ppm. ¹³C NMR (151 MHz, DMSO-d₆) δ 173.36, 159.21, 157.98, 152.07, 150.48, 139.55, 125.57, 86.20, 85.24, 79.19, 78.96, 71.45, 70.70, 69.67, 58.48, 58.45, 51.15, 40.67, 34.55, 33.26, 31.21, 29.02, 28.97, 28.94, 28.89, 28.87, 28.67, 28.45, 26.82, 26.57, 26.46, 26.40, 24.43, 22.06, 13.91 ppm. HRMS calc. for C₃₇H₆₄N₇O₆ [M+H]⁺ 702.4918, found 702.4937.

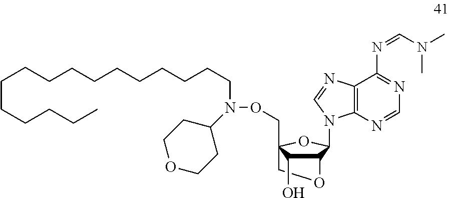 [SMILES: CCCCCCCCCCCCCCCCN(OC[C@]12CO[C@@H](C1O)[C@H](n1cnc3c(/N=C\N(C)C)ncnc31)O2)C1CCOCC1]

N′-[9-[(4R,6R)-4-[[hexadecyl(tetrahydropyran-4-yl)aminooxymethyl]-7-hydroxyl-2,5-dioxabicyclo[2.21]heptan-6-yl]purin-6-yl]-N,N-dimethyl-formamidine (41): To a clear solution of 39 (0.19 g, 246.07 mol) in THF (5 mL) was added TBAF (83.64 mg, 319.89 mol) in single portion and stirred for 10 hr at 22° C. All the volatile matters were evaporated under high vacuum pump and the crude residue thus obtained, was purified by flash column chromatography (gradient: 0-7% MeOH in DCM) to afford 41 (0.126 g, 78% yield) as white foam. ¹H NMR (600 MHz, DMSO-d₆) δ 8.91 (s, 1H), 8.42 (s, 1H), 8.24 (s, 1H), 5.94 (s, 1H), 5.79 (d, J=4.4 Hz, 1H), 4.42 (s, 1H), 4.28 (d, J=4.5 Hz, 1H), 4.13 (d, J=11.0 Hz, 1H), 4.02-3.93 (m, 2H), 3.89-3.79 (m, 4H), 3.26 (td, J=11.9, 2.2 Hz, 2H), 3.20 (s, 3H), 3.13 (s, 3H), 2.77 (tt, J=11.1, 3.8 Hz, 1H), 2.68 (hept, J=6.5 Hz, 2H), 1.77-1.70 (m, 2H), 1.58-1.43 (m, 5H), 1.30-1.10 (m, 17H), 0.85 (t, J=7.0 Hz, 3H) ppm. ¹³C NMR (151 MHz, DMSO-d6) δ 159.21, 157.97, 152.08, 150.44, 139.44, 125.60, 86.18, 85.31, 78.92, 71.39, 70.48, 70.41, 66.06, 66.04, 61.85, 54.13, 40.68, 34.56, 31.30, 29.05, 29.03, 29.02, 28.98, 28.94, 28.91, 28.72, 26.87, 26.11, 22.10, 13.96 ppm. HRMS calc. for C₃₅H₆₀N₇O₆[M+H]⁺ 658.4656, found 658.4641.

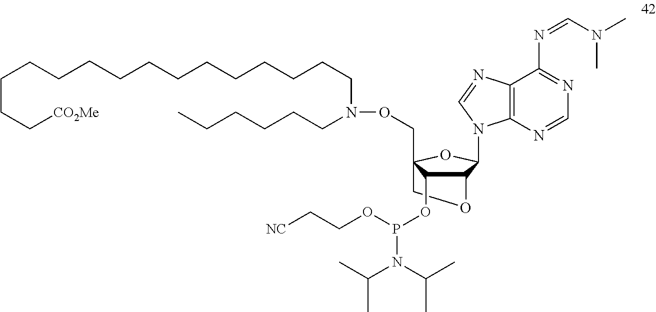 [SMILES: CCCCCCN(CCCCCCCCCCCCCCCC(=O)OC)OC[C@]12CO[C@@H](C1OP(OCCC#N)N(C(C)C)C(C)C)[C@H](n1cnc3c(/N=C\N(C)C)ncnc31)O2]

Methyl-16-[[(4R,6R)-7-[2-cyanoethoxy(dimethylamino)phosphanyl]oxy-6-[6-[(Z)-dimethyl aminomethyleneamino]purin-9-yl]-2,5-dioxabicyclo[2.2.1]heptan-4-yl]methoxy-hexyl-amino]hexadecanoate (42): To a clear solution of 40 (0.28 g, 398.90 mol) in DCM (15 mL) was added NMI (65.50 mg, 797.79 mol, 63.59 μL) and DIPEA (257.77 mg, 1.99 mmol, 347.39 μL) in single portions. After stirring the reaction mixture for 5 minutes at 22° C., 2-cyanoethyl-N,N-diisopropylchlorophosphoramidite (188.82 mg, 797.79 mol) was added and continued stirring for 1 hr and TLC was checked. Starting material was consumed and reaction mixture was diluted with DCM (15 mL). DCM layer was washed with 10% NaHCO₃ (2×20 mL) solution, and brine (20 mL). Organic layer was separated, dried over anhydrous Na₂SO₄, filtered and filtrate was evaporated at 36° C. to afford crude compound which was purified by flash chromatography (0-3% MeOH in DCM containing 3% TEA) to afford 42 (0.29 g, 80% yield) as white foam.

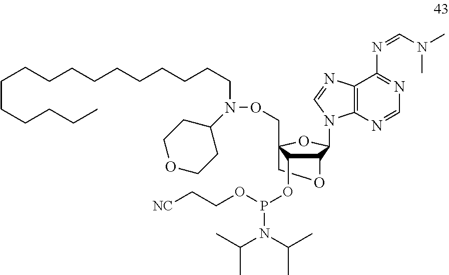 [SMILES: CCCCCCCCCCCCCCCCN(OC[C@]12CO[C@@H](C1OP(OCCC#N)N(C(C)C)C(C)C)[C@H](n1cnc3c(/N=C\N(C)C)ncnc31)O2)C1CCOCC1]

N′-[9-[(4R,6R)-7-[2-cyanoethoxy-(diisopropylamino)phosphanyl]oxy-4-[[hexadecyl(tetrahydro pyran-4-yl)amino]oxymethyl]-2,5-dioxabicyclo[2.2.1]heptan-6-yl]purin-6-yl]-N,N-dimethyl-formamidine (43): To a clear solution of 41 (0.2 g, 304.00 mol) in DCM (20 mL) was added NMI (49.92 mg, 608.01 mol, 48.46 μL) and DIPEA (196.45 mg, 1.52 mmol, 264.75 μL) in single portions. After stirring the reaction mixture for 5 minutes at 22° C., 2-cyanoethyl-N,N-diisopropylchlorophosphoramidite (143.90 mg, 608.01 mol, 135.76 μL) was added and continued stirring for 1 hr and TLC was checked. Starting material was consumed and reaction mixture was diluted with DCM (15 mL). DCM layer was washed with 10% NaHCO₃ (2×25 mL) solution, and brine (30 mL). Organic layer was separated, dried over anhydrous Na₂SO₄, filtered and filtrate was evaporated at 36° C. to afford crude compound which was purified by flash chromatography (0-3% MeOH in DCM containing 3% TEA) to afford 43 (0.211 g, 81% yield) as white foam.

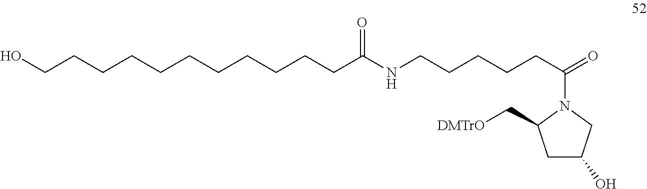

N-[6-[(2S,4R)-2-[[bis(4-methoxyphenyl)-phenyl-methoxy]methyl]-4-hydroxyl-pyrrolidin-1-yl]-6-oxo-hexyl]-12-hydroxyl-dodecanamide (52): To the clear solution of 12-hydroxyldodecanoic acid (0.8 g, 3.70 mmol) in dichloromethane (20 mL), DIPEA (1.45 g, 11.09 mmol, 1.95 mL) was added at 25° C. and stirred for 5 minutes. To this clear solution, was added 513 (1.97 g, 3.70 mmol) and (1-cyano-2-ethoxy-2-oxoethylidenaminooxy)dimethylamino-morpholino-carbenium hexafluorophosphate (COMU) (1.96 g, 4.44 mmol) in single portion and stirred for 12 hr. TLC showed completion of reaction. Reaction mixture was diluted with DCM (30 mL) and washed with saturated NaHCO₃ (50 mL), aqueous NaCl (50 mL) and 20% Na₂S₂O₃ solution (50 mL). Organic layer was then separated, dried over anhydrous Na₂SO₄, filtered and the filtrate was evaporated to dryness. Crude compound thus obtained, was purified by flash column chromatography (gradient; 1-15% methanol in DCM) to afford 52 (1.94 g, 72% yield). ¹H NMR (500 MHz, CDCl₃) δ 7.43 (d, J=7.4 Hz, 1H), 7.37-7.23 (m, 13H), 7.21-7.15 (m, 3H), 6.87-6.74 (m, 7H), 5.86-5.66 (m, 2H), 5.47 (s, 1H), 4.64-4.46 (m, 1H), 4.45-4.32 (m, 2H), 4.13 (dd, J=8.4, 4.8 Hz, 1H), 3.87-3.74 (m, 11H), 3.72-3.57 (m, 6H), 3.57-3.39 (m, 4H), 3.32-3.10 (m, 5H), 3.03 (dd, J=11.4, 4.7 Hz, 1H), 2.41-2.18 (m, 4H), 2.18-2.04 (m, 5H), 1.78-1.42 (m, 8H), 1.39-1.23 (m, 31H) ppm. ¹³C NMR (126 MHz, CDCl₃) δ 174.83, 173.74, 173.71, 173.57, 171.98, 171.95, 158.76, 158.70, 158.52, 158.40, 147.48, 145.60, 145.22, 144.70, 139.62, 136.94, 136.44, 136.31, 135.86, 135.81, 130.21, 130.15, 130.13, 130.10, 129.27, 128.35, 128.21, 128.16, 128.03, 127.97, 127.91, 127.87, 127.79, 127.20, 127.08, 126.85, 126.76, 126.66, 113.34, 113.30, 113.18, 113.17, 113.08, 86.68, 86.00, 85.75, 81.56, 70.57, 70.55, 69.34, 69.32, 67.19, 67.14, 63.70, 63.64, 63.10, 60.11, 60.09, 56.53, 55.96, 55.89, 55.39, 55.34, 54.12, 39.05, 38.93, 37.10, 37.01, 36.98, 36.95, 35.04, 34.86, 32.88, 30.24, 29.71, 29.70, 29.68, 29.64, 29.58, 29.50, 29.48, 29.45, 29.42, 29.40, 29.36, 29.33, 26.49, 26.46, 26.12, 26.10, 25.96, 25.91, 25.89, 25.82, 24.32 ppm. HRMS calc. for C₄₄H₆₂N₂O₇Na [M+Na]⁺ 753.4455, found 753.4470.

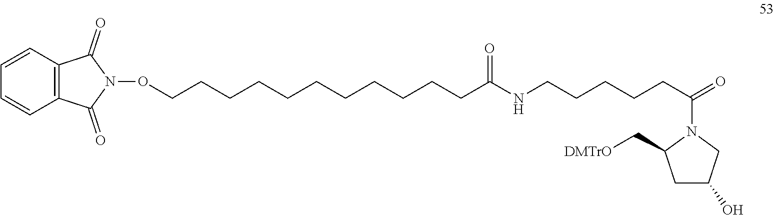

N-[6-[(2S,4R)-2-[[bis(4-methoxyphenyl)-phenyl-methoxy]methyl]-4-hydroxyl-pyrrolidin-1-yl]-6-oxo-hexyl]-12-[(1,3-dioxo-3a,7a-dihydroisoindol-2-yl)oxy]dodecanamide (53): To a clear solution of 52 (1.5 g, 2.05 mmol) in dry DMF (20 mL) was added triphenylphosphine (706.78 mg, 2.67 mmol) and N-hydroxylphthalimide (448.64 mg, 2.67 mmol) single portions. To this resulting mixture, diethyl azodicarboxylate (474.06 mg, 2.67 mmol, 495.88 μL) was added at 0° C. dropwise for 15 minutes. Ice bath was removed, and reaction mixture was stirred for 12 hr. To the reaction mixture, EtOAc (50 mL) and water (50 mL) were added and stirred. Organic layer was separated, washed with brine (30 mL), dried over anhydrous Na₂SO₄, filtered and the filtrate was evaporated to dryness. Crude mass obtained, was purified by flash column chromatography (gradient: 0-5% MeOH in DCM) to afford 53 (1 g, 56% yield) as hygroscopic solid. ¹H NMR (400 MHz, CDCl₃) δ 7.88-7.69 (m, 5H), 7.41-7.11 (m, 10H), 6.80 (td, J=9.1, 2.3 Hz, 4H), 5.94-5.61 (m, 1H), 4.68-4.46 (m, 1H), 4.40 (dq, J=8.4, 4.8 Hz, 1H), 4.19 (t, J=6.8 Hz, 2H), 3.87-3.61 (m, 7H), 3.60-3.37 (m, 2H), 3.32-3.08 (m, 4H), 2.84 (d, J=4.6 Hz, 1H), 2.55 (d, J=4.5 Hz, 1H), 2.38-2.02 (m, 5H), 1.98 (dd, J=13.4, 8.5 Hz, 1H), 1.78 (q, J=6.7 Hz, 4H), 1.69-1.37 (m, 8H), 1.33-1.25 (m, 19H) ppm. ¹³C NMR (101 MHz, CDCl₃) δ 173.51, 173.41, 172.63, 171.92, 163.83, 158.67, 158.49, 145.21, 144.70, 136.42, 136.29, 135.84, 135.80, 134.56, 130.32, 130.11, 130.08, 129.08, 128.97, 128.19, 128.14, 128.01, 127.86, 127.06, 126.83, 123.60, 113.31, 113.15, 86.64, 85.97, 78.77, 70.53, 69.32, 65.54, 63.71, 56.52, 55.92, 55.87, 55.38, 55.31, 54.11, 39.23, 39.02, 38.45, 36.97, 36.83, 34.83, 33.28, 29.58, 29.56, 29.53, 29.46, 29.43, 29.39, 29.35, 28.26, 26.53, 26.47, 25.93, 25.64, 24.72, 24.29 ppm. HRMS calc. for C₅₂H₆₆N₃O₉ [M+H]⁺ 876.4799, found 876.4788.

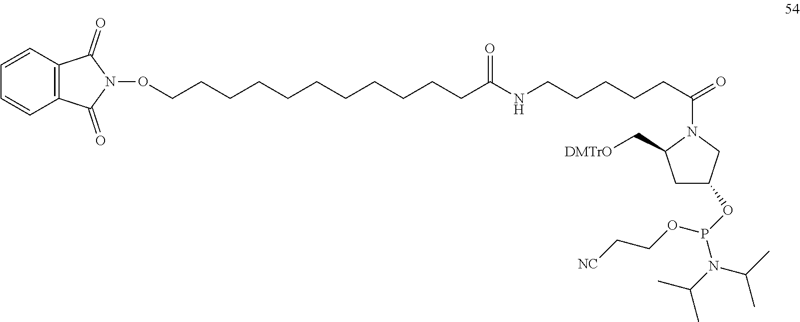

N-[6-[2-[[bis(4-methoxyphenyl)-phenyl-methoxy]methyl]-4-[2-cyanoethoxy-(diisopropylamino)phosphanyl]oxy-pyrrolidin-1-yl]-6-oxo-hexyl]-12-(1,3-dioxoisoindolin-2-yl)oxy-dodecanamide (54): To a clear solution of 53 (0.3 g, 342.43 mol) in dichloromethane (25 mL), DIPEA (223.52 mg, 1.71 mmol, 301.23 μL) and NMI (56.80 mg, 684.87 mol, 55.14 μL) were added at 22° C. To this reaction mixture, 2-cyanoethyl-N,N-diisopropylchlorophosphoramidite (170.63 mg, 684.87 mol, 160.97 μL) was added slowly after 5 minutes and stirred for 1 hr. Reaction mixture was diluted with DCM (10 mL) and quenched with 10% NaHCO₃ solution (20 mL). Organic layer was separated, dried on anhydro Na₂SO₄, filtered and filtrate was evaporated to dryness. The crude compound was thus obtained was purified by flash column chromatography (gradient: 10-60% EtOAc in hexane) to afford 54 (0.22 g, 60% yield) as white hygroscopic solid. ¹H NMR (400 MHz, CDCl₃) δ 7.87-7.79 (m, 2H), 7.78-7.71 (m, 2H), 7.39-7.33 (m, 2H), 7.28-7.15 (m, 9H), 6.87-6.76 (m, 4H), 4.67 (dd, J=10.2, 5.2 Hz, 1H), 4.35 (d, J=8.9 Hz, 1H), 4.20 (t, J=6.8 Hz, 2H), 3.92-3.71 (m, 9H), 3.65-3.45 (m, 3H), 3.36 (ddd, J=9.2, 4.5, 1.7 Hz, OH), 3.21-3.03 (m, 1H), 2.72-2.55 (m, 6H), 2.40-2.12 (m, 2H), 1.85-1.69 (m, 2H), 1.63 (s, 8H), 1.46 (q, J=7.4 Hz, 2H), 1.33-1.11 (m, 46H) ppm. ¹³C NMR (101 MHz, CDCl₃) δ 163.83, 158.69, 158.51, 145.25, 136.27, 134.56, 130.18, 130.10, 129.12, 128.21, 128.05, 127.90, 126.82, 123.61, 113.33, 113.19, 86.04, 78.79, 60.54, 58.17, 55.38, 55.33, 45.28, 45.15, 43.39, 43.27, 29.68, 29.64, 29.61, 29.58, 29.46, 28.29, 25.68, 24.81, 24.73, 24.61, 23.96, 21.63, 21.20, 20.57, 20.50, 20.44, 14.34, 0.13 ppm. ³¹P NMR (202 MHz, CD₃CN) δ 147.26, 147.23, 147.06, 147.04, 147.01, 146.73 ppm. HRMS calc. for C₆₁H₈₃N₅O₁₀P [M+H]⁺ 1076.5878, found 1076.5920.

### REFERENCES

1. Gottlieb, H. E.; Kotlyar, V.; Nudelman, A., NMR Chemical Shifts of Common Laboratory Solvents as Trace Impurities. The Journal of Organic Chemistry 1997, 62 (21), 7512-7515.
2. Manoharan, M.; Rajeev, K. G.; Yamada, T.; Butler, D.; Jayaprakash, K. N.; Jayraman, M.; Matsuda, S.; Pandey, R. K.; Peng, C. G. Preparation of monomers and oligonucleotides comprising triazolyl cycloaddition adducts via click chemical for treating various disorders and diseases. US20120035115A1, 2012.
3. Nair, J. K.; Willoughby, J. L. S.; Chan, A.; Charisse, K.; Alam, M. R.; Wang, Q.; Hoekstra, M.; Kandasamy, P.; Kel'in, A. V.; Milstein, S.; Taneja, N.; O'Shea, J.; Shaikh, S.; Zhang, L.; van der Sluis, R. J.; Jung, M. E.; Akinc, A.; Hutabarat, R.; Kuchimanchi, S.; Fitzgerald, K.; Zimmermann, T.; van Berkel, T. J. C.; Maier, M. A.; Rajeev, K. G.; Manoharan, M., Multivalent N-Acetylgalactosamine-Conjugated siRNA Localizes in Hepatocytes and Elicits Robust RNAi-Mediated Gene Silencing. J. Am. Chem. Soc. 2014, 136 (49), 16958-16961.
4. Dai, Y.; Cai, X.; Bi, X.; Liu, C.; Yue, N.; Zhu, Y.; Zhou, J.; Fu, M.; Huang, W.; Qian, H., Synthesis and anti-cancer evaluation of folic acid-peptide-paclitaxel conjugates for addressing drug resistance. European Journal of Medicinal Chemistry 2019, 171, 104-115.
5. Witt, T.; Haeussler, M.; Kulpa, S.; Mecking, S., Chain Multiplication of Fatty Acids to Precise Telechelic Polyethylene. Angew. Chem., Int. Ed. 2017, 56 (26), 7589-7594.
6. Iwasaki, T.; Tajimi, Y.; Kameda, K.; Kingwell, C.; Wcislo, W.; Osaka, K.; Yamawaki, M.; Morita, T.; Yoshimi, Y., Synthesis of 23-, 25-, 27-, and 29-Membered (Z)-Selective Unsaturated and Saturated Macrocyclic Lactones from 16- and 17-Membered Macrocyclic Lactones and Bromoalcohols by Wittig Reaction, Yamaguchi Macrolactonization, and Photoinduced Decarboxylative Radical Macrolactonization. J. Org. Chem. 2019, 84 (12), 8019-8026.
7. Genet, J. P.; Kahn, P., A new synthesis of 14-tetradecanolide from cyclododecanone. Tetrahedron Lett. 1980, 21 (16), 1521-4.
8. Clark, J. L.; Hollecker, L.; Mason, J. C.; Stuyver, L. J.; Tharnish, P. M.; Lostia, S.; McBrayer, T. R.; Schinazi, R. F.; Watanabe, K. A.; Otto, M. J.; Furman, P. A.; Stec, W. J.; Patterson, S. E.; Pankiewicz, K. W., Design, synthesis, and antiviral activity of 2′-deoxy-2′-fluoro-2′-C-methylcytidine, a potent inhibitor of hepatitis C virus replication. J Med Chem 2005, 48 (17), 5504-8.
9. Parmar, R. G.; Brown, C. R.; Matsuda, S.; Willoughby, J. L. S.; Theile, C. S.; Charisse, K.; Foster, D. J.; Zlatev, I.; Jadhav, V.; Maier, M. A.; Egli, M.; Manoharan, M.; Rajeev, K. G., Facile Synthesis, Geometry, and 2′-Substituent-Dependent in Vivo Activity of 5′-(E)- and 5′-(Z)-Vinylphosphonate-Modified siRNA Conjugates. Journal of Medicinal Chemistry 2018, 61 (3), 734-744.
10. Inoue, H.; Hayase, Y.; Imura, A.; Iwai, S.; Miura, K.; Ohtsuka, E., Synthesis and hybridization studies on two complementary nona(2′-O-methyl)ribonucleotides. Nucleic Acids Res. 1987, 15 (15), 6131-48.
11. Koshkin, A. A.; Singh, S. K.; Nielsen, P.; Rajwanshi, V. K.; Kumar, R.; Meldgaard, M.; Olsen, C. E.; Wengel, J., LNA (locked nucleic acids): synthesis of the adenine, cytosine, guanine, 5-methylcytosine, thymine and uracil bicyclonucleoside monomers, oligomerization, and unprecedented nucleic acid recognition. Tetrahedron 1998, 54 (14), 3607-3630.
12. Obika, S.; Uneda, T.; Sugimoto, T.; Nanbu, D.; Minami, T.; Doi, T.; Imanishi, T., 2′-0,4′-C-methylene bridged nucleic acid (2′,4′-BNA) synthesis and triplex-forming properties. Bioorg. Med. Chem. 2001, 9 (4), 1001-1011.
13. Koshkin, A. A.; Rajwanshi, V. K.; Wengel, J., Novel convenient syntheses of LNA [2.2.1]bicyclo nucleosides. Tetrahedron Lett. 1998, 39 (24), 4381-4384.
14. Koshkin, A. A.; Fensholdt, J.; Pfundheller, H. M.; Lomholt, C., A Simplified and Efficient Route to 2′-0, 4′-C-Methylene-Linked Bicyclic Ribonucleosides (Locked Nucleic Acid). J. Org. Chem. 2001, 66 (25), 8504-8512.
15. Wagner, D.; Verheyden, J. P. H.; Moffatt, J. G., Preparation and synthetic utility of some organo tin derivatives of nucleosides. J. Org. Chem. 1974, 39 (1), 24-30.

All patents and other publications identified in the specification and examples are expressly incorporated herein by reference for all purposes. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow. Further, to the extent not already indicated, it will be understood by those of ordinary skill in the art that any one of the various embodiments herein described and illustrated can be further modified to incorporate features shown in any of the other embodiments disclosed herein.

What is claimed is:

1.-55. (canceled)

56. An oligonucleotide comprising at least one nucleoside selected from the group consisting of:
a. nucleotides of Formula IIIa: 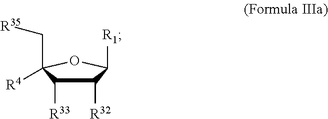
b. nucleotides of Formula IIIb: 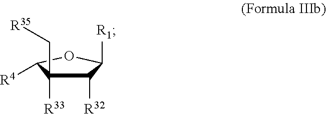
c. nucleotides of Formula IIIc: 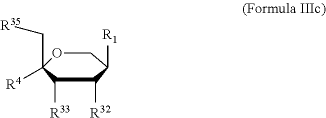
d. nucleotides of Formula IIId: 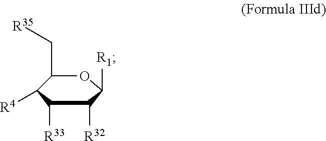
e. nucleotides of Formula IIIe: 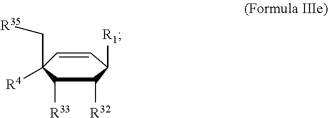
f. nucleotides of Formula IIIf: 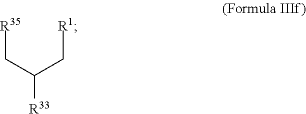
g. nucleotides of Formula IIIg: 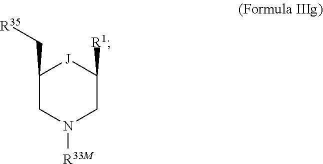
h. nucleotides of Formula IIIh: 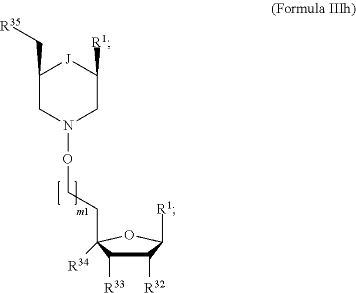
i. nucleotides of Forumla IVA; 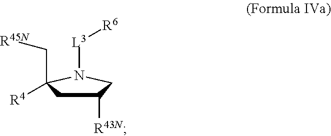
j. nucleotides of Formula IVb: 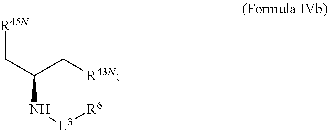
k. nucleotides of Formula IVc: 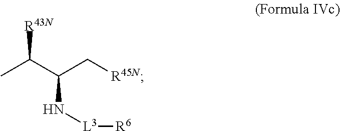
l. nucleotides of Formula IVd: 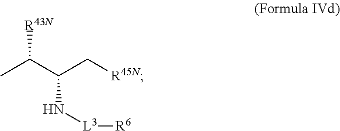
m. nucleotides of Formula IVe: 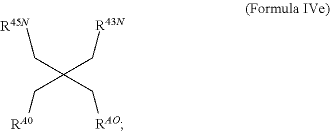
n. nucleotides of Formula IVf: 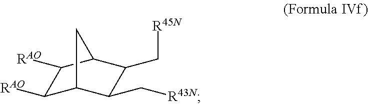
and
o. nucleotides of Formula IVg: 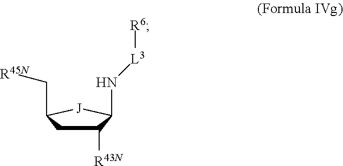
wherein:
m1 is 0, 1, 2, or 3 (e.g., 0 or 1);
J is O, S, CH₂ or N-alkyl (e.g., NCH₃);
R¹ is B or —B-L¹-R⁶;
L¹ is a linker;
L³ is a linker;
B is a nucleobase;
R³² is hydrogen, hydroxyl, halogen, protected hydroxyl, phosphate group, a reactive phosphorous group, optionally substituted C₁₋₃₀ alkyl, optionally substituted C₁₋₃₀haloalkyl, optionally substituted C₂₋₃₀alkenyl, optionally substituted C₂₋₃₀alkynyl, optionally substituted C₁₋₃₀ alkoxy (e.g., methoxy, 2-methoxyethoxy, dimethylaminoethoxyethyoxy, N-methylmethoxyamido), alkoxyalkyl (e.g., methoxyethyl), alkoxyalkylamine, alkoxyoxycarboxylate, amino, alkylamino, dialkylamino, —O—C₄₋₃₀alkyl-ON(CH₂R⁸)(CH₂R⁹), —O—C₄₋₃₀alkyl-ON(CH₂R⁸)(CH₂R⁹), a bond to an internucleotide linkage to a subsequent nucleotide, a 3′-oligonuclotide capping group, a ligand, a solid support, a linker, a linker covalently bonded (e.g., —OC(O)CH₂CH₂C(O)—) to a solid support, or —Z-L²-R⁶;
each Z is independently absent, a bond, O, S, or NR^{NR6};
each R^{NR6} is independently H, optionally substituted C₁₋₃₀ alkyl, optionally substituted C₂₋₃₀alkenyl, optionally substituted C₂₋₃₀alkynyl, or a nitrogen protecting group;
each L² is a linker;
R³³ is hydrogen, hydroxyl, halogen, protected hydroxyl, phosphate group, a reactive phosphorous group, optionally substituted C₁₋₃₀ alkyl, optionally substituted C₁₋₃₀haloalkyl, optionally substituted C₂₋₃₀alkenyl, optionally substituted C₂₋₃₀alkynyl, optionally substituted C₁₋₃₀ alkoxy (e.g., methoxy, 2-methoxyethoxy, dimethylaminoethoxyethyoxy, N-methylmethoxyamido), alkoxyalkyl (e.g., methoxyethyl), alkoxyalkylamine, alkoxyoxycarboxylate, amino, alkylamino, dialkylamino, —O—C₄₋₃₀alkyl-ON(CH₂R⁸)(CH₂R⁹), —O—C₄₋₃₀alkyl-ON(CH₂R⁸)(CH₂R⁹), a bond to an internucleotide linkage to a subsequent nucleotide, a 3′-oligonuclotide capping group, a ligand, a solid support, a linker, a linker covalently bonded (e.g., —OC(O)CH₂CH₂C(O)—) to a solid support, or —Z-L²-R⁶, and optionally, only one of R³ and R³ is a phosphate group, a reactive phosphorous group, a solid support, a linker, a linker covalently bonded (e.g., —OC(O)CH₂CH₂C(O)—) to a solid support, or a bond to an internucleotide linkage to a subsequent nucleotide;
R⁴ is hydrogen, optionally substituted C₁₋₆alkyl, optionally substituted C₁₋₃₀haloalkyl, optionally substituted C₂₋₆alkenyl, optionally substituted C₂₋₆alkynyl, optionally substituted C₁₋₆alkoxy, or —Z-L²-R⁶;
or R⁴ and R³² taken together are 4′-C(R¹⁰R¹¹)ᵥ—Y-2′ or 4′-Y—C(R¹⁰R¹¹)ᵥ-2′;
Y is —O—, —CH₂—, —CH(Me)-, —C(CH₃)₂—, —S—, —N(R¹²)—, —C(O)—, —C(S)—, —S(O)—, —S(O)₂—, —OC(O)—, —C(O)O—, —N(R¹²)C(O)—, or —C(O)N(R¹²)—;
R¹⁰ and R¹¹ independently are H, optionally substituted C₁-C₆alkyl, optionally substituted C₁₋₃₀haloalkyl, optionally substituted C₂-C₆alkenyl or optionally substituted C₂-C₆alkynyl;
R¹² is hydrogen, optionally substituted C₁₋₃₀alkyl, optionally substituted C₁-C₃₀alkoxy, C₁₋₄haloalkyl, optionally substituted C₂₋₄alkenyl, optionally substituted C₂-4alkynyl, optionally substituted C₁₋₃₀alky-CO₂H, or a nitrogen-protecting group;
v is 1, 2 or 3;
or R⁴ and R³³ taken together with the atoms to which they are attached form an optionally substituted C₃₋₈cycloalkyl, optionally substituted C₃₋₈cycloalkenyl, or optionally substituted 3-8 membered heterocyclyl;
R³⁵ is R⁶, —Z-L²-R⁶, a bond to an internucleotide linkage to a preceding nucleotide, hydrogen, hydroxyl, protected hydroxyl, phosphate group, optionally substituted C₁₋₃₀ alkyl, optionally substituted C₁₋₃₀haloalkyl, optionally substituted C₂₋₃₀alkenyl, optionally substituted C₂₋₃₀alkynyl, optionally substituted C₁₋₃₀ alkoxy, halogen, alkoxyalkyl (e.g., methoxyethyl), alkoxyalkylamine, alkoxyoxycarboxylate, amino, alkylamino, dialkylamino, —O—C₄₋₃₀alkyl-ON(CH₂R⁸)(CH₂R⁹), —O—C₄₋₃₀alkyl-ON(CH₂R⁸)(CH₂R⁹), vinylphosphonate (VP) group, C₃₋₆cycloalkylphosphonate (e.g., cyclopropylphosphonate), monophosphate ((HO)₂(0)P—O-5′), diphosphate ((HO)₂(0)P—O—P(HO)(O)—O-5′), triphosphate ((HO)₂(0)P—O—(HO)(O)P—O—P(HO)(O)—O-5′); monothiophosphate (phosphorothioate, (HO)₂(S)P—O-5′), monodithiophosphate (phosphorodithioate; (HO)(HS)(S)P—O-5′), phosphorothiolate ((HO)₂(0)P—S-5′); alpha-thiotriphosphate; beta-thiotriphosphate; gamma-thiotriphosphate; phosphoramidates ((HO)₂(O)P—NH-5′, (HO)(NH₂)(O)P—O-5′), alkylphosphonates (R(OH)(O)P—O-5′, R=alkyl, e.g., methyl, ethyl, isopropyl, propyl, etc. . . . ), alkyletherphosphonates (R(OH)(O)P—O-5′, R=alkylether, e.g., methoxymethyl (CH₂OMe), ethoxymethyl, etc. . . . ), (HO)₂(X)P—O[—(CH₂)ₐ—O—P(X)(OH)—O]_{b}-5′ or (HO)₂(X)P—O[—(CH₂)ₐ—P(X)(OH)—O]_{b}-5′ or (HO)₂(X)P—[—(CH₂)ₐ—O—P(X)(OH)—O]_{b}-5′, where X is O, S or optionally substituted alkyl, and dialkyl terminal phosphates and phosphate mimics (e.g., HO[—(CH₂)ₐ—O—P(X)(OH)—O]_{b}-5′, H₂N[—(CH₂)ₐ—O—P(X)(OH)—O]_{b}-5′, H[—(CH₂)ₐ—O—P(X)(OH)—O]_{b}-5′, Me₂N[—(CH₂)ₐ—O—P(X)(OH)—O]_{b}-5′, HO[—(CH₂)ₐ—P(X)(OH)—O]_{b}-5′, H₂N[—(CH₂)ₐ—P(X)(OH)—O]_{b}-5′, H[—(CH₂)ₐ—P(X)(OH)—O]_{b}-5′, Me₂N[—(CH₂)ₐ—P(X)(OH)—O]_{b}-5′, wherein X is O or S; and a and b are each independently 1-10);
each R⁶ is independently —O—N(R⁷)R^{7′}, —O—N═C(R⁷)R^{7′}, ═N—OR⁷, —N(R⁷)—OR^{7′} or —N(R^{7′})—OR⁷;
each R⁷ and R^{7′} is independently H or a ligand, (e.g., a ligand selected independently from the group consisting of carbohydrates, lipids, vitamins, peptides, proteins, lipoproteins, peptidomimetics, polyamines, nucleosides and nucleotides, oligonucleotides, therapeutic agents, diagnostic agents, detectable labels, antibodies or fragments thereof, optionally substituted C₁₋₃₀ alkyl, optionally substituted C₁₋₃₀ alkenyl, optionally substituted C₁₋₃₀alkynyl, optionally substituted cycloalkyl, optionally substituted heterocyclyl, optionally substituted aryl, optionally substituted heteroaryl, polyethylene glycols (PEGs), nitrogen protecting group, optionally provided that at least one of R⁷ and R^{7′} is not H;
or R⁷ and R^{7′} together with the N they are attached to form an optionally substituted heterocyclyl (e.g., phthalimide or morpholine)
each R⁸ and R⁹ is independently H, a targeting ligand (e.g., GalNac), a pharmacokinetics modifier, optionally substituted C₁₋₃₀ alkyl, optionally substituted C₁₋₃₀ alkenyl, or optionally substituted C₁₋₃₀alkynyl; and
R^{33M} is hydrogen, hydroxyl, protected hydroxyl, phosphate group, a reactive phosphorous group, optionally substituted C₁₋₃₀ alkyl, optionally substituted C₁₋₃₀haloalkyl, optionally substituted C₂₋₃₀alkenyl, optionally substituted C₂₋₃₀alkynyl, optionally substituted C₁₋₃₀ alkoxy (e.g., methoxy), alkoxyalkyl (e.g., methoxyethyl), alkoxyalkylamine, alkoxyoxycarboxylate, —O—C₄₋₃₀alkyl-ON(CH₂R⁸)(CH₂R⁹), —O—C₄₋₃₀alkyl-ON(CH₂R⁸)(CH₂R⁹), a solid support, a linker, a linker covalently bonded (e.g., —OC(O)CH₂CH₂C(O)—) to a solid support, a bond to an internucleotide linkage to a subsequent nucleotide, or —Z—R^{33L};
each Z is independently absent, a bond, O, S, or NR^{NR6};
each R^{NR6} is independently H, optionally substituted C₁₋₃₀ alkyl, optionally substituted C₁₋₃₀haloalkyl, optionally substituted C₂₋₃₀alkenyl, optionally substituted C₂₋₃₀alkynyl, or a nitrogen protecting group;
each R^{33L} is a ligand, a linker covalently linked to one or more ligands, optionally substituted C₁₋₃₀ alkyl, optionally substituted C₂₋₃₀alkenyl, optionally substituted C₂₋₃₀alkynyl, or polyethylene glycol
R^{43N} hydrogen, hydroxyl, halogen, protected hydroxyl, phosphate group, a reactive phosphorous group, optionally substituted C₁₋₃₀ alkyl, optionally substituted C₁₋₃₀haloalkyl, optionally substituted C₂₋₃₀alkenyl, optionally substituted C₂₋₃₀alkynyl, optionally substituted C₁₋₃₀ alkoxy (e.g., methoxy, 2-methoxyethoxy, dimethylaminoethoxyethyoxy, N-methylmethoxyamido), alkoxyalkyl (e.g., methoxyethyl), alkoxyalkylamine, alkoxyoxycarboxylate, amino, alkylamino, dialkylamino, —O—C₄₋₃₀alkyl-ON(CH₂R⁸)(CH₂R⁹), —O—C₄₋₃₀alkyl-ON(CH₂R⁸)(CH₂R⁹), a bond to an internucleotide linkage to a subsequent nucleotide, a 3′-oligonuclotide capping group, a ligand, a solid support, a linker, a linker covalently bonded (e.g., —OC(O)CH₂CH₂C(O)—) to a solid support;
R^{45N} is a bond to an internucleotide linkage to a preceding nucleotide, hydrogen, hydroxyl, protected hydroxyl, phosphate group, optionally substituted C₁₋₃₀ alkyl, optionally substituted C₁₋₃₀haloalkyl, optionally substituted C₂₋₃₀alkenyl, optionally substituted C₂₋₃₀alkynyl, optionally substituted C₁₋₃₀ alkoxy, halogen, alkoxyalkyl, alkoxyalkylamine, alkoxyoxycarboxylate, amino, alkylamino, dialkylamino, —O—C₄-30alkyl-ON(CH₂R⁸)(CH₂R⁹), —O—C₄₋₃₀alkyl-ON(CH₂R⁸)(CH₂R⁹), vinylphosphonate (VP) group, C3-6cycloalkylphosphonate, monophosphate ((HO)₂(O)P—O-5′), diphosphate ((HO)₂(O)P—O—P(HO)(O)—O-5′), triphosphate ((HO)₂(O)P—O—(HO)(O)P—O—P(HO)(O)—O-5′); monothiophosphate (phosphorothioate, (HO)₂(S)P—O-5′), monodithiophosphate (phosphorodithioate; (HO)(HS)(S)P—O-5′), phosphorothiolate ((HO)2(O)P—S-5′); alpha-thiotriphosphate; beta-thiotriphosphate; gamma-thiotriphosphate; phosphoramidates, alkylphosphonates), alkyletherphosphonates), (HO)₂(X)P—O[—(CH₂)ₐ—O—P(X)(OH)—O]_{b}-5′ or (HO)2(X)P—O[—(CH₂)ₐ—P(X)(OH)—O]_{b}-5′ or (HO)₂(X)P—[—(CH₂)ₐ—O—P(X)(OH)—O]_{b}-5′, where X is O, S or optionally substituted alkyl, and HO[—(CH₂)ₐ—O—P(X)(OH)—O]—5′, H₂N[—(CH₂)ₐ—O—P(X)(OH)—O]_{b}-5′, H[—(CH₂)ₐ—O—P(X)(OH)—O]_{b}-5′, Me₂N[—(CH₂)ₐ—O—P(X)(OH)—O]_{b}-5′, HO[—(CH₂)ₐ—P(X)(OH)—O]_{b}-5′, H₂N[—(CH₂)ₐ—P(X)(OH)—O]_{b}-5′, H[—(CH₂)ₐ—P(X)(OH)—O]_{b}-5′, Me₂N[—(CH₂)ₐ—P(X)(OH)—O]_{b}-5′, wherein X is O or S; and a and b are each independently 1-10;
each R^{AO} is independently R⁶ or —Z-L²-R⁶, and
provided that when the oligonucleotide comprises a nucleotide of formulae IIIa-IIg, the oligonucleotide comprises at least one R⁶ group.

57. The oligonucleotide of claim 56, wherein R¹ is —B-L¹-R⁶.

58. The oligonucleotide of claim 56, wherein R¹ is a pyrimidine nucleobase comprising -L¹-R⁶ at the C₅ position; or R¹ is a pyrimidine nucleobase comprising -L¹-R⁶ at the N₄ position; or R¹ is a purine nucleobase comprising -L¹-R⁶ at the N₂, N₆, or C8 position; or R¹ is a N₇-deaza purine nucleobase comprising -L¹-R⁶ at the N2, N6, C8 or N7-deaza position.

59. The oligonucleotide of claim 56, wherein L¹ is a bond, optionally substituted C₁-C₂₀alkylene, optionally substituted C₂-C₂₀alkenylene or optionally substituted C₂-C₂₀alkynylene, and where the backbone of the alkylene, alkenylene or alkynylene can be interrupted or terminated by O, S, S(O), SO₂, NR^{N1}, NR^{N1}—C(O), C(O), C(O)O, cleavable linking group, substituted or unsubstituted aryl, substituted or unsubstituted heteroaryl, substituted or unsubstituted heterocyclic; where R^{N1} is hydrogen, acyl, aliphatic or substituted aliphatic.

60. The oligonucleotide of claim 56, wherein R³² is a bond to an internucleotide linkage to a subsequent nucleotide, —Z-L²-R⁶, hydrogen, hydroxyl, protected hydroxyl, halogen, optionally substituted C₁₋₃₀ alkoxy (e.g., methoxy, 2-methoxyethoxy, dimethylaminoethoxyethyoxy, N-methylmethoxyamido), halogen, alkoxyalkyl (e.g., 2-methoxyethyl), amino, alkylamino, dialkylamino, a reactive phosphorous group, a solid support, a linker or a linker covalently attached to a solid support; or R² and R⁴ taken together are 4′-C(R¹⁰R¹¹)ᵥ-Y-2′ or 4′-Y—C(R¹⁰R¹¹)ᵥ-2′.

61. (canceled)

62. (canceled)

63. (canceled)

64. (canceled)

65. (canceled)

66. (canceled)

67. (canceled)

68. The oligonucleotide of claim 56, wherein R³³ is a bond to an internucleotide linkage to a subsequent nucleotide, —Z-L²-R⁶, hydrogen, hydroxyl, protected hydroxyl, a solid support, a linker, or a linker covalently attached to a solid support.

69. (canceled)

70. (canceled)

71. The oligonucleotide of claim 56, wherein R³⁵ is R⁶, a bond to an internucleotide linkage to a preceding nucleotide, hydroxyl, protected hydroxy, optionally substituted C₁₋₃₀ alkoxy, vinylphosphonate (VP) group, monophosphate, diphosphate, triphosphate, monothiophosphate (phosphorothioate), monodithiophosphate, phosphorothiolate, alpha-thiotriphosphate, beta-thiotriphosphate, gamma-thiotriphosphate, phosphoramidates, alkylphosphonates, alkyletherphosphonates, dialkyl terminal phosphate, or phosphate mimic.

72. (canceled)

73. The oligonucleotide of claim 56, wherein R³⁵ is a bond to an internucleotide linkage to a preceding nucleotide.

74. The oligonucleotide of claim 56, wherein R³⁵ is R⁶.

75. The oligonucleotide of claim 56, wherein R³⁵ is hydroxyl or protected hydroxyl.

76. (canceled)

77. (canceled)

78. (canceled)

79. (canceled)

80. (canceled)

81. (canceled)

82. (canceled)

83. (canceled)

84. (canceled)

85. (canceled)

86. The oligonucleotide of claim 56, wherein Z is O.

87. The oligonucleotide of claim 56, wherein R⁶ is —O-N(R⁷)R^{7′} or —O-N═C(R⁷)R^{7′}.

88. (canceled)

89. The oligonucleotide of claim 56, wherein at least one of R⁷ and R^{7′} is selected from the group consisting of carbohydrates, lipids, vitamins, peptides, proteins, lipoproteins, peptidomimetics, polyamines, nucleosides and nucleotides, oligonucleotides, therapeutic agents, diagnostic agents, detectable labels, antibodies or fragments thereof, optionally substituted C₁₋₃₀ alkyl, optionally substituted C₁₋₃₀ alkenyl, optionally substituted C₁₋₃₀alkynyl, optionally substituted cycloalkyl, optionally substituted heterocyclyl, optionally substituted aryl, optionally substituted heteroaryl, polyethylene glycols (PEGs), and nitrogen protecting groups.

90. (canceled)

91. The oligonucleotide of claim 56, wherein L² is a bond, optionally substituted C₁-C₂₀alkylene, optionally substituted C₂-C₂₀alkenylene or optionally substituted C₂-C₂₀alkynylene, and where the backbone of the alkylene, alkenylene or alkynylene can be interrupted or terminated by O, S, S(O), SO₂, NR^{N1}, NR^{N1}—C(O), C(O), C(O)O, cleavable linking group, substituted or unsubstituted aryl, substituted or unsubstituted heteroaryl, substituted or unsubstituted heterocyclic; where R^{N1} is hydrogen, acyl, aliphatic or substituted aliphatic.

92. The oligonucleotide of claim 56, wherein the nucleoside at the 5′-end of the oligonucleotide comprises a R⁶ group.

93. The oligonucleotide of claim 56, wherein the nucleoside at the 5′-end of the oligonucleotide comprises a nucleoside selected from Formulae IIIa-IIIg.

94. (canceled)

95. The oligonucleotide of claim 93, wherein one of R³² and R³³ is a bond to a modified internucleotide linkage.

96. The oligonucleotide of claim 56, wherein the oligonucleotide comprises at its 3′-end a 3′-oligonuclotide capping group, a ligand, a solid support, a linker, a linker covalently bonded (e.g., —OC(O)CH₂CH₂C(O)—) to a solid support, hydrogen or hydroxyl.

97. The oligonucleotide of claim 56, wherein at least one of R³², R³³, R³⁵, R^{43N} and R^{45N} is a bond to a modified internucleotide linkage.

98. (canceled)

99. The oligonucleotide of claim 56, wherein the oligonucleotide comprises from 3 to 50 nucleotides, and one or more of (i) at least one ribonucleotide; (ii) at least one 2′-deoxyribonucleotide; (iii) at least one nucleotide with a modified or non-natural nucleobase, (iv) at least one nucleotide with a modified ribose sugar; (v) at least one nucleotide comprising a group other than H or OH at the 2′-position of the ribose sugar; (v) at least one nucleotide with a 2′-F ribose; (vii) at least one nucleotide with a 2′-OMe ribose; (viii) at least one nucleotide comprising a moiety other than a ribose sugar; or (ix) at least one modified internucleotide linkage.

100. (canceled)

101. (canceled)

102. (canceled)

103. (canceled)

104. (canceled)

105. (canceled)

106. (canceled)

107. (canceled)

108. (canceled)

109. (canceled)

110. The oligonucleotide of claim 56, wherein the oligonucleotide is attached to a solid support or the oligonucleotide comprises at least one hydroxyl, phosphate or amino protecting group.

111. (canceled)

112. A double-stranded nucleic acid comprising a first oligonucleotide strand and a second oligonucleotide strand substantially complementary to the first strand, wherein the first or second strand is an oligonucleotide of claim 56.

113. The double-stranded nucleic acid of claim 112, wherein the first and second strand are independently 15 to 25 nucleotides in length.

114. The double-stranded nucleic acid of claim 112, wherein double-stranded nucleic acid is capable of inducing RNA interference.

115. (canceled)

116. (canceled)

117. (canceled)

118. A method of reducing the expression of a target gene in a subject, comprising administering to the subject either:
(i) a double-stranded RNA according to claim 112, wherein the first strand or the second strand is complementary to a target gene.
(ii).

119.-172. (canceled)
